(19) United States
(12) Patent Application Publication
(10) Pub. No.: US 2024/0270699 A1
(43) Pub. Date: Aug. 15, 2024

(54) COMPOUNDS HAVING N-ARYLPYRIMIDIN-2-AMINE DERIVATIVES AS THERAPEUTIC AGENTS

(71) Applicant: Lomond Therapeutics, Inc., San Diego, CA (US)

(72) Inventors: Volodymyr Kysil, San Diego, CA (US); Vladislav Zenonovich Parchinsky, Moscow (RU); Alexei Pushechnikov, San Diego, CA (US); Alexandre Vasilievich Ivachtchenko, Hallandale Beach, FL (US); Nikolay Savchuk, San Diego, CA (US)

(21) Appl. No.: 18/604,472

(22) Filed: Mar. 13, 2024

Related U.S. Application Data
(63) Continuation of application No. PCT/US22/42431, filed on Sep. 2, 2022.
(60) Provisional application No. 63/244,775, filed on Sep. 16, 2021.

(51) Int. Cl. C07D 239/48; A61K 31/505; A61K 31/506; A61K 31/55; A61K 31/5513; C07D 239/50; C07D 401/12; C07D 401/14; C07D 403/04; C07D 403/12; C07D 403/14; C07D 409/14; C07D 411/12; C07D 413/04; C07D 413/14; C07D 417/04; C07D 417/14

(52) U.S. Cl. CPC C07D 239/48; A61K 31/505; A61K 31/506; A61K 31/55; A61K 31/5513; C07D 239/50; C07D 401/12; C07D 401/14; C07D 403/04; C07D 403/12; C07D 403/14; C07D 409/14; C07D 411/12; C07D 413/04; C07D 413/14; C07D 417/04; C07D 417/14

(57) ABSTRACT

The present invention is generally directed to inhibitors of hematopoietic progenitor kinase 1 (HPK1), leucine rich repeat kinase 2 (LRRK2) protein, FMS-like tyrosine kinase 3 (FLT3) gene, interleukin-1 receptor-associated kinase 1 (IRAK1), interleukin-1 receptor-associated kinase 4 (IRAK4), and Janus kinases (JAKs), including Janus kinase 1 (JAK1), Janus kinase 2 (JAK2), Janus kinase 3 (JAK3), and tyrosine kinase 2 (TYK2) useful in the treatment of diseases and disorders modulated by said HPK1, LRRK2, FLT3, IRAK1, IRAK4, and JAKs, having the Formula (I):

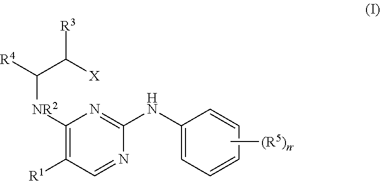

### CROSS-REFERENCE TO RELATED APPLICATIONS

This application claims priority to and the benefit of U.S. Provisional Patent Application Ser. No. 63/244,775 filed Sep. 16, 2021 and entitled “Compounds Having N-arylpyrimidin-2-amine derivatives as therapeutic agents”, and International Patent Application Serial No. PCT/US2022/042431 filed Sep. 2, 2022 and entitled “Compounds Having N-arylpyrimidin-2-amine derivatives as therapeutic agents” the disclosures of which are incorporated herein by reference in their entirety for all purposes.

### FIELD OF INVENTION

The present invention is directed to inhibitors of hematopoietic progenitor kinase 1 (HPK1), leucine rich repeat kinase 2 (LRRK2) protein, FMS-like tyrosine kinase 3 (FLT3) gene, interleukin-1 receptor-associated kinase 1 (IRAK1), interleukin-1 receptor-associated kinase 4 (IRAK4), and Janus kinases (JAKs), including Janus kinase 1 (JAK1), Janus kinase 2 (JAK2), Janus kinase 3 (JAK3), and tyrosine kinase 2 (TYK2). The inhibitors described herein can be useful in the treatment of diseases or disorders associated with HPK1, LRRK2, FLT3, IRAK1, IRAK4, and JAKs, such as cancer, autoimmune disease, inflammatory disease, viral infection, male fertility control, benign hyperplasia, sepsis, vascular disorder, atherosclerotic disease, and neurodegenerative disorder. In particular, the invention is concerned with compounds and pharmaceutical compositions inhibiting HPK1, LRRK2, FLT3, IRAK1, IRAK4, and JAKs, methods of treating diseases or disorders associated with HPK1, LRRK2, FLT3, IRAK1, IRAK4, and JAKs, and methods of synthesizing these compounds.

### BACKGROUND OF THE INVENTION

Hematopoietic progenitor kinase 1 (HPK1) is a hematopoietic cell-restricted Ste20 serine/threonine kinase. HPK1 kinase activity can be induced by activation signals generated by various different cell surface receptors found in hematopoietic cells upon ligand engagement. Ligand engagement or antibody-mediated crosslinking of T cell receptors (TCR), B cell antigen receptor (BCR), transforming growth factor β receptor (TGF-βR), erythropoietin receptor (EPOR), and Fas can induce HPK1 kinase activity. Each receptor utilizes unique, but sometimes overlapping, signaling mechanisms to activate HPK1. HPK1 acts as a down-modulator of T and B cell functions through the AP-1, NFKB, Erk2, and Fas pathways. For example, HPK1 has been implicated as a negative regulator of signal transduction in T-cells through phosphorylation and activation of the T-cell receptor adaptor protein SLP-76, which leads to subsequent downregulation of the AP-1 and Erk2 pathways. In B-cells, HPK1 downregulates B-cell receptor (BCR) signaling through phosphorylation of the SLP-76 paralog BLINK.

Thus, HPK1 is viewed as a possible target for therapeutic intervention. For example, it has been reported that HPK1 can be a novel target for cancer immunotherapy (Sawasdikosol et al., Immunol Res. 2012 December; 54(1-3):262-5). Specifically, targeted disruption of HPK1 alleles confers T cells with an elevated Th1 cytokine production in response to TCR engagement. HPK1 (−/−) T cells proliferate more rapidly than the haplotype-matched wild-type counterpart and are resistant to prostaglandin E2 (PGE(2))-mediated suppression. Most strikingly, mice that received adoptive transfer of HPK1 (−/−) T cells became resistant to lung tumor growth. Also, the loss of HPK1 from dendritic cells (DCs) endows them with superior antigen presentation ability, enabling HPK1 (−/−) DCs to elicit a more potent anti-tumor immune response when used as cancer vaccine.

While full-length HPK1 can promote TCR-mediated activation of the nuclear factor kappa-light-chain-enhancer of activated B cells (NF-κB) pathway, the catalytically inactive cleavage product HPK1-C can suppress NF-κB activation upon TCR restimulation, leading to activation-induced cell death (AICD) (Brenner et al., EMBO J. 2005, 24:4279). Taking together the catalytic and non-catalytic roles of HPK1, it is possible that blocking the HPK1 kinase activity with a small-molecule inhibitor may promote activation of B- and T-cells, leading to superior anti-tumor immunity, while also facilitating AICD, helping to maintain peripheral immune tolerance.

In the publication US 2020/0390776 A1 described invention generally relates to isofuranone compounds (for example to the compound of formula 1.1) that modulate or inhibit the enzymatic activity of hematopoietic progenitor kinase 1 (HPK1). It was described that compounds have activity as HPK1 inhibitors and have selectivity over IRAK-4.

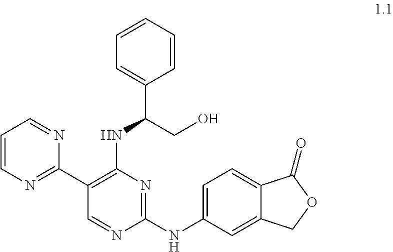 [SMILES: O=C1OCc2cc(Nc3ncc(-c4ncccn4)c(N[C@H](CO)c4ccccc4)n3)ccc21]

Janus kinases (JAKs) are cytoplasmic tyrosine kinases that transduce cytokine signaling from membrane receptors to STAT transcription factors. Four JAK family members are described, JAK1, JAK2, JAK3, and TYK2. Upon binding of the cytokine to its receptor, JAK family members auto- and/or transphosphorylate each other, followed by phosphorylation of STATs that then migrate to the nucleus to modulate transcription. JAK-STAT intracellular signal transduction serves the interferons, most interleukins, as well as a variety of cytokines and endocrine factors such as EPO, TPO, GH, OSM, LIF, CNTF, GM-CSF and PRL. A combination of genetic models and small molecule JAK inhibitor research revealed the therapeutic potential of JAK inhibitors (JAKinibs).

Using TYK2 knock out mice, it has been shown that IL-6, IL-10, IL-11, IL12, IL-13, IL-19, IL-20, IL-22, IL-23, IL-27, IL-28, IL-29, IL-31, IL-35, and/or type 1 interferons signaling are dependent on TYK2. However, it has recently been shown that JAK1 is a key driver in IFNa, IL6, IL10, and IL22 signaling, TYK2 is involved in type I interferons (including IFNa, IFNb), IL23 and IL12 signaling. Since the activity of IL12 and IL23 is particularly increased in patients with auto-immune diseases such as psoriasis, systemic lupus erythematosus (SLE), psoriatic arthritis, and/or inflammatory bowel disorders, selective TYK2 inhibition may be particularly advantageous in the treatment of these diseases while avoiding JAK2 dependent erythropoietin (EPO) and thrombopoietin (TPO) signaling. Additionally, due to its involvement in type I interferons (including IFNa, IFNb) signaling, TYK2 inhibition may be particularly useful in the treatment of the cytokine storm associated with COVID-19 infections.

Furthermore, a variant of TYK2 gene described in the general human population, with a modification in one amino acid in the kinase domain of TYK2 which invalidates its kinase activity, is associated with the decreased risk of autoimmune and inflammatory diseases.

Leucine rich repeat kinase 2 (LRRK2) is a member of the ROCO protein family and all members of this family share five conserved domains. There is evidence to show that the increased LRRK2 kinase activity is associated with neuronal toxicity in cell culture models (Smith et al., 2006 Nature Neuroscience 9: 1231-1233) and kinase inhibitor compounds protect against LRRK2-mediated cell death (Lee et al., 2010 Nat. Med. 16: 998-1000).

Additional evidence links LRRK2 function and dysfunction with autophagy-lysosomal pathways (Manzoni and Lewis, 2013 Faseb J. 27:3234-3429). LRRK2 proteins confer defects in chaperone-mediated autophagy that negatively impact the ability of cells to degrade alpha-synuclein (Orenstein et al., 2013 Nature Neurosci. 16 394-406). In other cell models, selective LRRK2 inhibitors have been shown to stimulate macroautophagy (Manzoni et al., 2013 BBA Mol. Cell Res. 1833: 2900-2910). These data suggest that small molecule inhibitors of LRRK2 kinase activity may have utility in the treatment of diseases characterized by defects in cellular proteostasis that result from aberrant autophagy/lysosomal degradation pathways including forms of Parkinson's disease associated with GBA mutations (Swan and Saunders-Pullman 2013 Curr. Neurol. Neurosci Rep. 13: 368), other alpha-synucleinopathies, tauopathies, Alzheimer's disease (Li et al., 2010 Neurodegen. Dis. 7: 265-271) and other neurodegenerative diseases (Nixon 2013 Nat. Med. 19: 983-997).

Other studies have also shown that overexpression of the G2019S mutant form of LRRK2 confers defects in subventricular zone (SVZ) neuroprogenitor cell proliferation and migration in transgenic mouse models (Winner et al., 2011 Neurobiol. Dis. 41: 706-716) and reduces neurite length and branching cell culture models (Dachsel et al., 2010 Parkinsonism & Related Disorders 16: 650-655). Moreover, it was reported that agents that promote SVZ neuroprogenitor cell proliferation and migration also improve neurological outcomes following ischemic injury in rodent models of stroke (Zhang et al., 2010 J. Neurosci. Res. 88: 3275-3281). These findings suggest that compounds that inhibit aberrant activity of LRRK2 may have utility for the treatments designed to stimulate restoration of CNS functions following neuronal injury, such as ischemic stroke, traumatic brain injury, spinal cord injury.

Mutations in LRRK2 have also been identified that are clinically associated with the transition from mild cognitive impairment (MCI) to Alzheimer's disease (WO2007149798). These data suggest that inhibitors of LRRK2 kinase activity may be useful for the treatment diseases such as Alzheimer's disease, other dementias and related neurodegenerative disorders.

Aberrant regulation of normal LRRK2 proteins is also observed in some disease tissues and models of disease. Normal mechanisms of translational control of LRRK2 by miR-205 are perturbed in some sporadic PD cases, where significant decreases in miR-205 levels in PD brain samples concur with elevated LRRK2 protein levels in those samples (Cho et al., (2013) Hum. Mol. Gen. 22: 608-620). Therefore, LRRK2 inhibitors may be used in treatment of sporadic PD patients who have elevated levels of normal LRRK2 proteins. In an experimental model of Parkinson's disease in marmosets, an elevation of LRRK2 mRNA is observed in a manner that correlates with the level of L-Dopa induced dyskinesia (Hurley, M. J et al., 2007 Eur. J. Neurosci. 26: 171-177). This suggests that LRRK2 inhibitors may have a utility in amelioration of such dyskinesias.

Significantly elevated levels of LRRK2 mRNA have been reported in ALS patient muscle biopsy samples (Shtilbans et al., 2011 Amyotrophic Lateral Sclerosis 12: 250-256) It is suggested that elevated levels of LRRK2 kinase activity may be a characteristic feature of ALS. Therefore, this observation indicated that LRRK2 inhibitor may have utility for treatment of ALS.

There is also evidence indicating that LRRK2 kinase activity may play a role in mediating microglial proinflammatory responses (Moehle et al., 2012, J. Neuroscience 32:1602-1611). This observation suggests a possible utility of LRRK2 inhibitors for treatment of aberrant neuroinflammatory mechanisms that contribute a range of neurodegenerative diseases, including Parkinson's disease, Alzheimer's disease, multiple sclerosis, HIV-induced dementia, amyotrophic lateral sclerosis, ischemic stroke, traumatic brain injury and spinal cord injury. Some evidence also indicates that LRRK2 plays a role in regulating neuronal progenitor differentiation in vitro (Milosevic, J. et al., 2009 Mol. Neurodegen. 4: 25). This evidence suggests that inhibitors of LRRK2 may have a utility in production of neuronal progenitor cells in vitro for consequent therapeutic application in cell-based treatment of CNS disorders.

Meta-analysis of three genome wide associated scans for Crohn's disease identified a number of loci associated with the disease, including the locus containing the LRRK2 gene (Barrett et al., 2008, Nature Genetics, 40: 955-962). Evidence has also emerged that LRRK2 is an IFN-γ target gene that may be involved in signaling pathways relevant to Crohn's disease pathogenesis (Gardet et al., 2010, J. Immunology, 185: 5577-5585). These findings suggest that inhibitors of LRRK2 may have utility in the treatment of Crohn's disease.

As an IFN-γ target gene, LRRK2 may also play a role in T cell mechanisms that underlie other diseases of the immune system such as multiple sclerosis and rheumatoid arthritis. Further potential utility of LRRK2 inhibitors comes from the reported finding that B lymphocytes constitute a major population of LRRK2 expressing cells (Maekawa et al. 2010, BBRC 392: 431-435). This suggests that LRRK2 inhibitors may be effective in treatment of diseases of the immune system for which B cell depletion is, or may be, effective in diseases such as lymphomas, leukemias, multiple sclerosis (Ray et al., 2011 J. Immunol. 230: 109), rheumatoid arthritis, systemic lupus erythematosus, autoimmune hemolytic anemia, pure red cell aplasia, idiopathic thrombocytopenia purpura (ITP), Evans syndrome, vasculitis, bullous skin disorders, type 1 diabetes mellitus, Sjogren's syndrome, Devic's disease and inflammatory myopathies (Engel et al., 2011 Pharmacol. Rev. 63: 127-156; Homam et al., 2010 J. Clin. Neuromuscular Disease 12: 91-102).

The FMS-like tyrosine kinase 3 (FLT3) gene encodes a membrane bound receptor tyrosine kinase that affects hematopoiesis leading to hematological disorders and malignancies. Activation of FLT3 receptor tyrosine kinases is initiated through the binding of the FLT3 ligand (FLT3L) to the FLT3 receptor, also known as Stem cell tyrosine kinase-1 (STK-1) and fetal liver kinase-2 (flk-2), which is expressed on hematopoietic progenitor and stem cells.

FLT3 is one of the most frequently mutated genes in hematological malignancies, present in approximately 30% of adult acute myeloid leukemia (AML). The presence of a FLT3 internal tandem duplication in patients with acute myeloid leukemia (AML) adds important prognostic information to cytogenetic risk group and response to the first cycle of chemotherapy. FLT3 mutations have been detected in approximately 2% of patients diagnosed with intermediate and high-risk myelodysplastic syndrome (MDS). Like MDS, the number of FLT3 mutations in patients with acute promyelocytic leukemia (APL) is small. The most common FLT3 mutations are internal tandem duplications (ITDs) that lead to in-frame insertions within the juxtamembrane domain of the FLT3 receptor. FLT3-ITD mutations have been reported in 15-35% of adult AML patients. Internal tandem duplication of FLT3 associated with leukocytosis in acute promyelocytic leukemia. A FLT3-ITD mutation is an independent predictor of poor patient prognosis and is associated with increased relapse risk after standard chemotherapy, and decreased disease free and overall survival. Prognostic implication of FLT3 and N-RAS gene mutations in acute myeloid leukemia. Less frequent are FLT3 point mutations that arise in the activation loop of the FLT3 receptor. The most commonly affected codon is aspartate 835 (D835). Nucleotide substitutions of the D835 residue occur in approximately 5-10% of adult acute myeloid leukemia patients.

The heightened frequency of constitutively activated mutant FLT3 in adult AML has made the FLT3 gene a highly attractive drug target in this tumor type. Several FLT3 inhibitors with varying degrees of potency and selectivity for the target have been or are currently being investigated and examined in AML patients.

Toll-like receptors (TLRs)/Interleukin 1-receptor (IL-1R) signaling engages IRAK4 and IRAK1 phosphorylation to drive downstream events such as NF-κB and interferon signaling in inflammation response and this process has been recently implicated in tumorigenesis. Moreover, pharmacologic inhibition of IRAK1/4 has been shown to be efficacious in targeting MDS and acute lymphoblastic leukemia (ALL) that carry IRAK1 activation through NF-κB-dependent or in-dependent mechanism.

Accordingly, there is a need for new compounds which would effectively and selectively inhibit hematopoietic progenitor kinase 1 (HPK1), leucine rich repeat kinase 2 (LRRK2) protein, FMS-like tyrosine kinase 3 (FLT3) gene, interleukin-1 receptor-associated kinase 1 (IRAK1), interleukin-1 receptor-associated kinase 4 (IRAK4), and Janus kinases (JAKs), including Janus kinase 1 (JAK1), Janus kinase 2 (JAK2), Janus kinase 3 (JAK3), and tyrosine kinase 2 (TYK2), thus allowing the design of specific treatments and dosages tailored to the pathology.

### SUMMARY OF THE INVENTION

A first aspect of the invention relates to compounds of Formula (I):

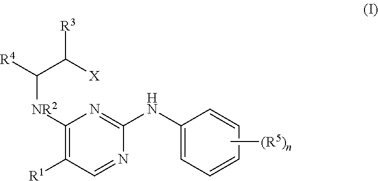

or pharmaceutically acceptable salts, solvates, prodrugs, enantiomers, stereoisomers, or tautomers thereof,
wherein:
X is H, halogen, or OH, provided that when R⁴ is unsubstituted phenyl and X is H then R¹ is C(O)OR⁶;
R¹ is selected from —CN, —NO₂, —C(O)NHR⁶, C(O)N(R⁶)₂, —C(O)OR⁶, —S(O)₂C₁₋₆ alkyl or a monocyclic heteroaryl comprising one or more heteroatoms independently selected from N, S, and O, wherein the heteroaryl is optionally substituted with one or more substituents selected from —OH, oxo, halogen, C₁₋₄ alkoxy, C₁₋₆ alkyl, C₂₋₆ alkenyl, or 4-7 membered monocyclic heterocycloalkyl, wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl;
R² is H or C₁₋₄ alkyl; or
R² and R⁸ together with the atoms to which they are attached and any intervening atoms, form a 5-to-6-membered heterocycle;
R³ is H; or
R³ and R⁸ together with the atoms to which they are attached and any intervening atoms, form a 5- to-6-membered cycloalkyl;
R⁴ is selected from —(CH₂)ₘ-aryl, —(CH₂)ₘ-heteroaryl, heterocyclyl, wherein the aryl, heteroaryl, or heteroaryl is optionally substituted with one or more R⁸;
each R⁵ is independently selected from halogen, C₁₋₆ alkyl, C₁₋₆ alkoxy, C₁₋₆ halogenalkyl, oxy C₁₋₆ alkyl, —S(O)₂—C₁₋₆ alkyl, —S(O)₂—C₂₋₆ alkenyl, —C(O)NH₂, —C(O)NH(C₁₋₆ alkyl), —C(O)N(C₁₋₆ alkyl)₂, —CH₂C(O)NH₂, —CH₂C(O)NH(C₁₋₆ alkyl), —CH₂C(O)N(C₁₋₆ alkyl)₂, —NHC(O)CH₃, aryl, heteroaryl; or
two R⁵ together with the atoms to which they are attached and any intervening atoms, form 6-7 membered heterocyclyl, or 5-7 membered heteroaryl, containing at least one heteroatom selected from N, O, S; wherein the cycloalkyl or heterocyclyl is optionally substituted with one or more R⁷;
each R⁶ is independently selected from H, OH, C₁₋₆ alkyl, —S(O)₂—C₁₋₆ alkyl, C₃₋₈ cycloalkyl, heterocyclyl, or heteroaryl, wherein the alkyl, alkoxy, heteroaryl, or heterocyclyl is optionally substituted with one or more R⁹; or
two R⁶ together with the atoms to which they are attached and any intervening atoms, form 4-8-membered heterocycle;
each R⁷ is independently selected from OH, halogen, C₁₋₆ alkyl, aryl, oxy C₁₋₆ alkyl, —CH₂OC(O)C₁₋₆ alkyl, or —CH₂OCH₂CH₂Si(CH₃)₃;
each R⁸ is independently selected from halogen, OH, NH₂, C₁₋₆ alkyl, —OC₁₋₆ alkyl, —NHC₁₋₆ alkyl, —N(C₁₋₆ alkyl)₂;
each R⁹ is independently selected from halogen, OH, NH₂, C₁₋₆ alkyl, C₁₋₆ alkoxy, C₁₋₆ haloalkyl, C₂₋₆ alkenyl, C₂₋₆ alkynyl, C₁₋₆ haloalkyl, C₃₋₈ cycloalkyl, heterocyclyl, aryl, or heteroaryl; and
m and n are integers independently selected from 0, 1, 2, 3, 4, 5 and 6.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with modulation of hematopoietic progenitor kinase 1 (HPK1). The method comprises administering to a patient in need of a treatment for diseases or disorders associated with modulation of HPK1 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting hematopoietic progenitor kinase 1 (HPK1). The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting hematopoietic progenitor kinase 1 (HPK1).

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting hematopoietic progenitor kinase 1 (HPK1).

Unexpectedly provided in this application compounds show higher activity, bioavailability and lower cytotoxicity compared to compounds described in US 2020/0390776 A1.

Another aspect of the invention relates to a method of treating a disease or disorder associated with modulation of leucine rich repeat kinase 2 (LRRK2) protein. The method comprises administering to a patient in need of a treatment for diseases or disorders associated with modulation of LRRK2 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting leucine rich repeat kinase 2 (LRRK2) protein. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting leucine rich repeat kinase 2 (LRRK2) protein.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting leucine rich repeat kinase 2 (LRRK2) protein.

Another aspect of the invention relates to a method of treating a disease or disorder associated with modulation of FMS-like tyrosine kinase 3 (FLT3) gene. The method comprises administering to a patient in need of a treatment for diseases or disorders associated with modulation of FLT3 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting FMS-like tyrosine kinase 3 (FLT3) gene. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting FMS-like tyrosine kinase 3 (FLT3) gene.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting FMS-like tyrosine kinase 3 (FLT3) gene.

Another aspect of the invention relates to a method of treating a disease or disorder associated with modulation of interleukin-1 receptor-associated kinase 1 (IRAK1). The method comprises administering to a patient in need of a treatment for diseases or disorders associated with modulation of IRAK1 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting interleukin-1 receptor-associated kinase 1 (IRAK1). The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting interleukin-1 receptor-associated kinase 1 (IRAK1).

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting interleukin-1 receptor-associated kinase 1 (IRAK1).

Another aspect of the invention relates to a method of treating a disease or disorder associated with modulation of interleukin-1 receptor-associated kinase 4 (IRAK4). The method comprises administering to a patient in need of a treatment for diseases or disorders associated with modulation of IRAK4 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting interleukin-1 receptor-associated kinase 4 (IRAK4). The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting interleukin-1 receptor-associated kinase 4 (IRAK4).

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting interleukin-1 receptor-associated kinase 4 (IRAK4).

Another aspect of the invention relates to a method of treating a disease or disorder associated with modulation of Janus kinases (JAKs), including Janus kinase 1 (JAK1), Janus kinase 2 (JAK2), Janus kinase 3 (JAK3), and tyrosine kinase 2 (TYK2). The method comprises administering to a patient in need of a treatment for diseases or disorders associated with modulation of JAKs an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting Janus kinases (JAKs), including Janus kinase 1 (JAK1), Janus kinase 2 (JAK2), Janus kinase 3 (JAK3), and tyrosine kinase 2 (TYK2). The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting Janus kinases (JAKs), including Janus kinase 1 (JAK1), Janus kinase 2 (JAK2), Janus kinase 3 (JAK3), and tyrosine kinase 2 (TYK2).

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting Janus kinases (JAKs), including Janus kinase 1 (JAK1), Janus kinase 2 (JAK2), Janus kinase 3 (JAK3), and tyrosine kinase 2 (TYK2).

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with modulation of hematopoietic progenitor kinase 1 (HPK1), comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of hematopoietic progenitor kinase 1 (HPK1) that are therapeutic agents in the treatment of diseases and disorders.

The present invention further provides compounds and compositions with an improved efficacy and safety profile relative to known hematopoietic progenitor kinase 1 (HPK1) inhibitors. The present disclosure also provides agents with novel mechanisms of action toward protein tyrosine phosphatase enzymes in the treatment of various types of diseases.

The present invention further provides methods of treating a disease or disorder associated with modulation of leucine rich repeat kinase 2 (LRRK2) protein, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of leucine rich repeat kinase 2 (LRRK2) protein that are therapeutic agents in the treatment of diseases and disorders.

The present invention further provides compounds and compositions with an improved efficacy and safety profile relative to known leucine rich repeat kinase 2 (LRRK2) protein inhibitors. The present disclosure also provides agents with novel mechanisms of action toward LRRK2 in the treatment of various types of diseases.

The present invention further provides methods of treating a disease or disorder associated with modulation of FMS-like tyrosine kinase 3 (FLT3) gene, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of FMS-like tyrosine kinase 3 (FLT3) gene that are therapeutic agents in the treatment of diseases and disorders.

The present invention further provides compounds and compositions with an improved efficacy and safety profile relative to known FMS-like tyrosine kinase 3 (FLT3) gene inhibitors. The present disclosure also provides agents with novel mechanisms of action toward FLT3 in the treatment of various types of diseases.

The present invention further provides methods of treating a disease or disorder associated with modulation of interleukin-1 receptor-associated kinase 1 (IRAK1), comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of interleukin-1 receptor-associated kinase 1 (IRAK1) that are therapeutic agents in the treatment of diseases and disorders.

The present invention further provides compounds and compositions with an improved efficacy and safety profile relative to known interleukin-1 receptor-associated kinase 1 (IRAK1) inhibitors. The present disclosure also provides agents with novel mechanisms of action toward IRAK1 in the treatment of various types of diseases.

The present invention further provides methods of treating a disease or disorder associated with modulation of interleukin-1 receptor-associated kinase 4 (IRAK4), comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of interleukin-1 receptor-associated kinase 4 (IRAK4) that are therapeutic agents in the treatment of diseases and disorders.

The present invention further provides compounds and compositions with an improved efficacy and safety profile relative to known interleukin-1 receptor-associated kinase 4 (IRAK4) inhibitors. The present disclosure also provides agents with novel mechanisms of action toward IRAK4 in the treatment of various types of diseases.

The present invention further provides methods of treating a disease or disorder associated with modulation of Janus kinases (JAKs), including Janus kinase 1 (JAK1), Janus kinase 2 (JAK2), Janus kinase 3 (JAK3), and tyrosine kinase 2 (TYK2), comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of Janus kinases (JAKs), including Janus kinase 1 (JAK1), Janus kinase 2 (JAK2), Janus kinase 3 (JAK3), and tyrosine kinase 2 (TYK2), that are therapeutic agents in the treatment of diseases and disorders.

The present invention further provides compounds and compositions with an improved efficacy and safety profile relative to known Janus kinase (JAK) inhibitors. The present disclosure also provides agents with novel mechanisms of action toward JAKs in the treatment of various types of diseases.

The present invention further provides methods of treating a disease, disorder, or condition selected from cancer, an autoimmune disease, an inflammatory disease, a viral infection, male fertility control, a benign hyperplasia, sepsis, a vascular disorder, an atherosclerotic disease, and a neurodegenerative disorder, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedure A).

In some aspects, the present disclosure provides an intermediate as described herein, being suitable for use in a method for preparing a compound as described herein (e.g., the intermediate is selected from the intermediates described in Example 1).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION OF THE INVENTION

The present disclosure relates to compounds and compositions that are capable of inhibiting the activity of hematopoietic progenitor kinase 1 (HPK1), leucine rich repeat kinase 2 (LRRK2) protein, FMS-like tyrosine kinase 3 (FLT3) gene, interleukin-1 receptor-associated kinase 1 (IRAK1), interleukin-1 receptor-associated kinase 4 (IRAK4), and Janus kinases (JAKs), including Janus kinase 1 (JAK1), Janus kinase 2 (JAK2), Janus kinase 3 (JAK3), and tyrosine kinase 2 (TYK2). The disclosure features methods of treating, preventing or ameliorating a disease or disorder in which HPK1, LRRK2, FLT3, IRAK1, IRAK4, and/or JAKs play(s) a role by administering to a patient in need thereof a therapeutically effective amount of a compound of Formula (I), or a pharmaceutically acceptable salt, hydrate, solvate, prodrug, stereoisomer, or tautomer thereof. The methods of the present invention can be used in the treatment of a variety of diseases, disorders, and conditions, including cancer, an autoimmune disease, an inflammatory disease, a viral infection, male fertility control, a benign hyperplasia, sepsis, a vascular disorder, an atherosclerotic disease, and a neurodegenerative disorder. In a first aspect of the invention, the compounds of Formula (I) are described:

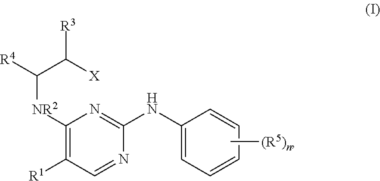

and pharmaceutically acceptable salts, hydrates, solvates, prodrugs, enantiomers, stereoisomers, and tautomers thereof, wherein n, R¹, R², R³, R⁴, R⁵, and X are described herein.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆) alkyl, and S(O)N((C₁-C₆) alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore, when containing two fused rings, the aryl groups herein defined may have a saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 24 ring atoms, containing one or more ring heteroatoms selected from N, O, S, P, Se, or B, the remaining ring atoms being C. Heteroaryl as herein defined also means a bicyclic heteroaromatic group wherein the heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ²-pyrrolo[2,1-b]pyrimidine, dibenzo[b,d] thiophene, pyridin-2-one, furo[3,2-c]pyridinyl, furo[2,3-c]pyridinyl, 1H-pyrido[3,4-b][1,4] thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with a fully unsaturated aromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated ring includes 0 to 4 heteroatoms selected from N, O, S, P, Se, or B, and is optionally substituted with one or more oxo. In heteroaryl ring systems containing more than two fused rings, a saturated or partially unsaturated ring may further be fused with a saturated or partially unsaturated ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, or benzo[c][1,2]oxaborol-1(3H)-olyl.

“Halogen” or “halo” refers to fluorine, chlorine, bromine, or iodine.

“Alkyl” refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, iso-pentyl, neo-pentyl, and iso-hexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, tert-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above-mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, adamantyl, and derivatives thereof. In the case of polycyclic cycloalkyl, only one of the rings in the cycloalkyl needs to be non-aromatic.

“Heterocyclyl”, “heterocycle” or “heterocycloalkyl” refers to a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms (such as O, N, S, P, Se, or B), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted one or more halogen. Examples of haloalkoxy groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

The term “amine” as used herein refers to primary (R—NH₂, R≠H), secondary (R₂—NH, R₂≠H) and tertiary (R₃—N, R≠H) amines. A substituted amine is intended to mean an amine where at least one of the hydrogen atoms has been replaced by the substituent.

The term “amino” as used herein means a substituent containing at least one nitrogen atom. Specifically, —NH₂, —NH(alkyl) or alkylamino, —N(alkyl)₂ or dialkylamino, amide-, carbamide-, urea, and sulfamide substituents are included in the term “amino”.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present invention also contemplates isotopically labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a disease or disorder in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

In some embodiments, the present disclosure is related to compounds of Formula I-A:

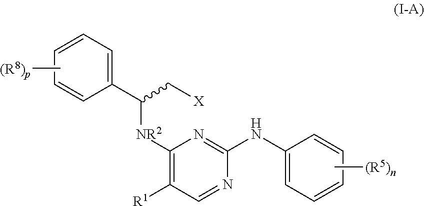

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-A1:

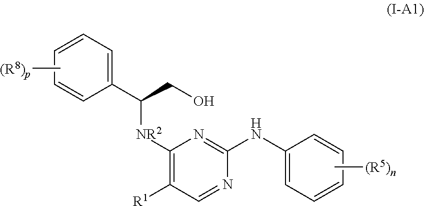

or pharmaceutically acceptable salts, solvates, prodrugs, enantiomers, stereoisomers, or tautomers thereof, wherein p is an integer from 0, 1, 2, 3, 4 and 5 and all other variables are as defined herein.

In some embodiments, the present disclosure is related to compounds of Formula I-A2:

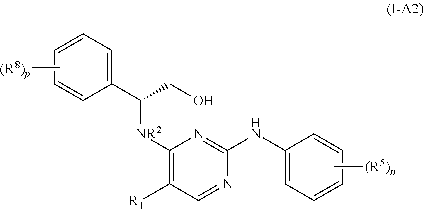

thereof, wherein p is an integer selected from 0, 1, 2, 3, 4 and 5 and all other variables are as defined herein.

In some embodiments, the present disclosure is related to compounds of Formula I-B1, I-B2, I-B3, I-B3′ or I-B4:

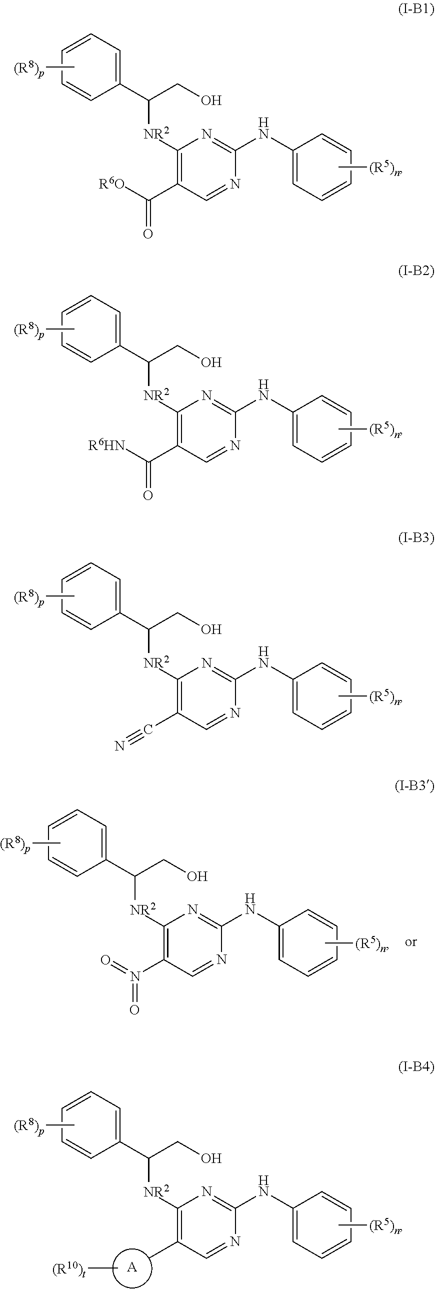

wherein Ring A is a monocyclic heteroaryl comprising one or more heteroatoms independently selected from N, S, and O; each R¹⁰ is independently selected from —OH, oxo, halogen, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁₋₆ alkyl)₂, —OH, —COOC₁₋₄ alkyl, —COOH, —CONH₂ or 4-7 membered monocyclic heterocycloalkyl; t is an integer selected from 0, 1, 2, 3 and 4; and p is an integer selected from 0, 1, 2, 3, 4 and 5, or pharmaceutically acceptable salts, solvates, prodrugs, enantiomers, stereoisomers, or tautomers thereof.

In some embodiments, the present disclosure is related to compounds of Formula I-B1-a:

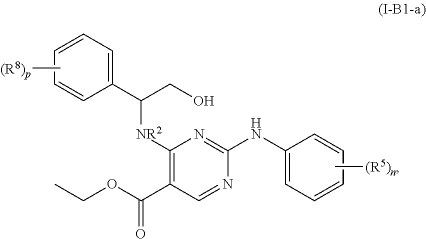

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B1-a*:

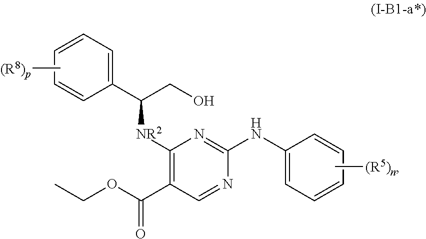

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B1-b:

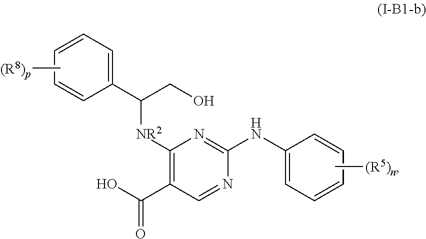

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B1-b*:

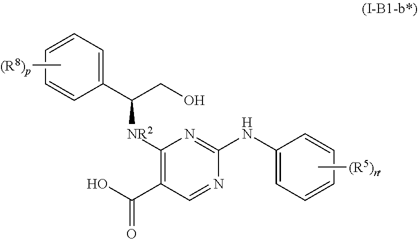

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B1-c:

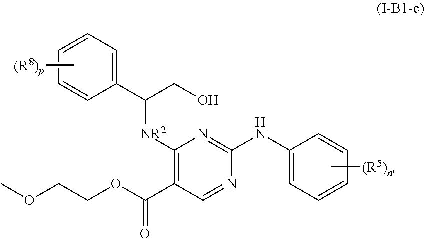

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B1-c*:

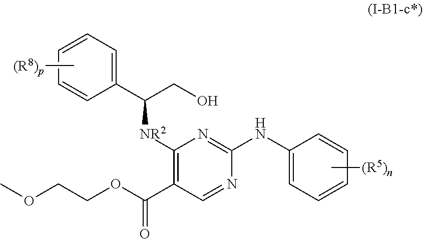

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B1-d:

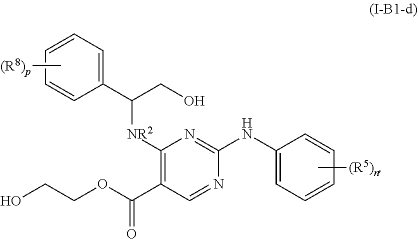

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B1-d*:

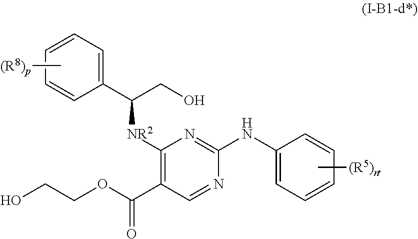

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-a:

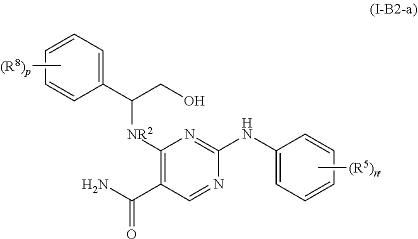

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-a*:

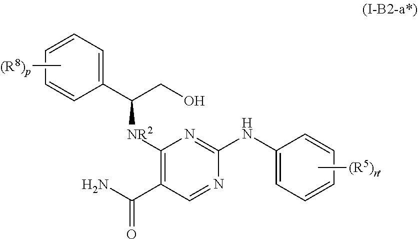

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-b:

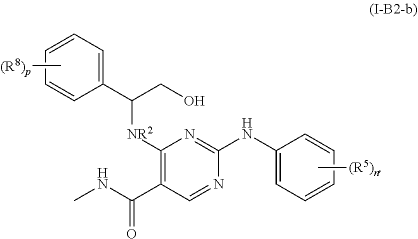

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-b*:

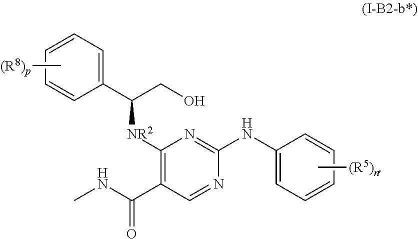

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-c:

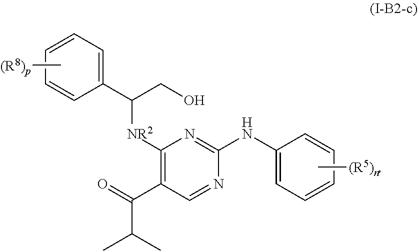

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-c*:

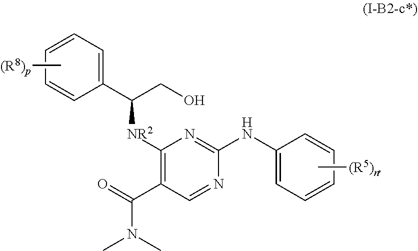

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-d:

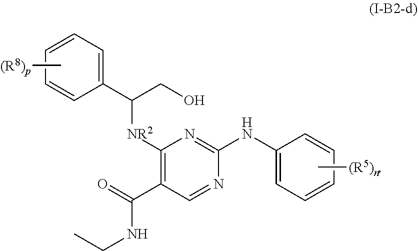

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-d*:

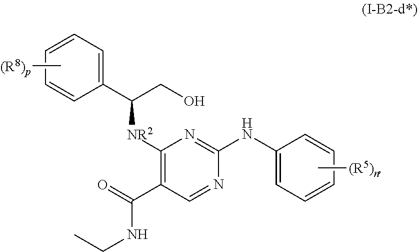

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-e:

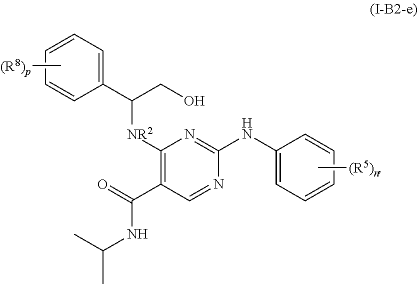

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-e*:

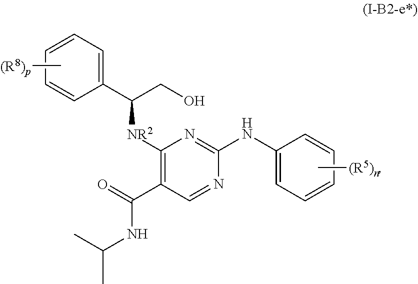

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-f:

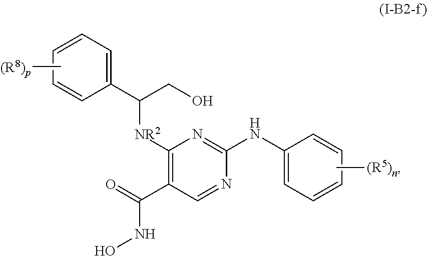

thereof, wherein p is an integer selected from 0, 1, 2, 3, 4 and 5 and all other variables are as defined herein.

In some embodiments, the present disclosure is related to compounds of Formula I-B2-f*:

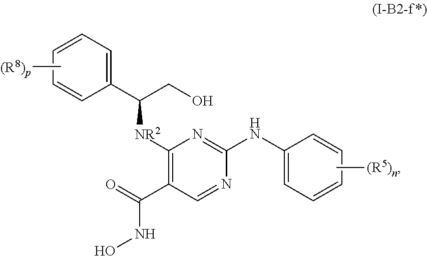

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-g:

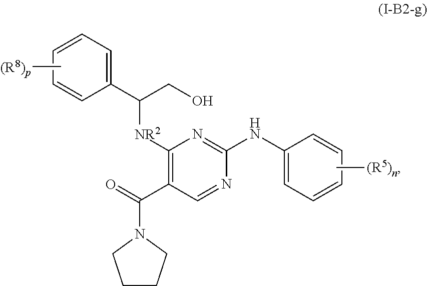

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-g*:

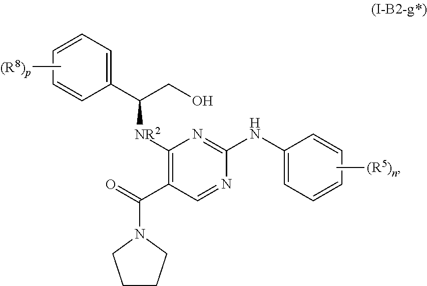

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-h:

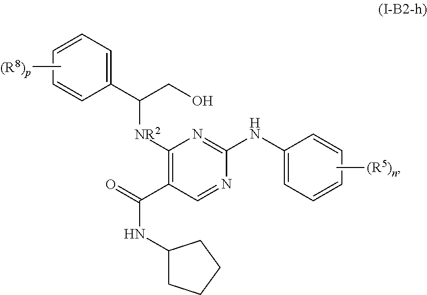

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-h*:

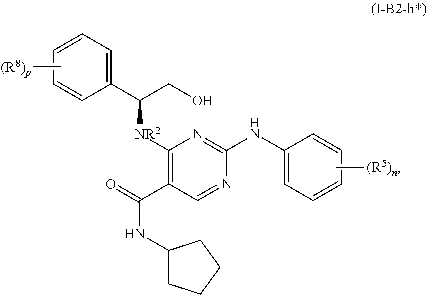

thereof, wherein p is an integer selected from 0, 1, 2, 3, 4 and 5 and all other variables are as defined herein.

In some embodiments, the present disclosure is related to compounds of Formula I-B2-i:

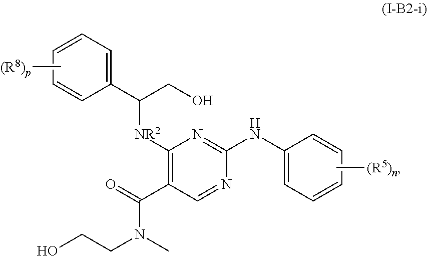

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-i*:

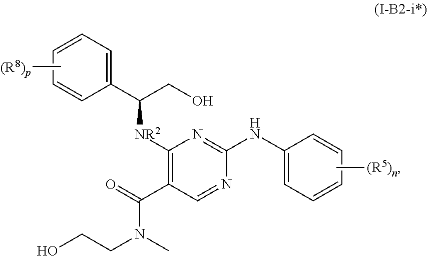

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-j:

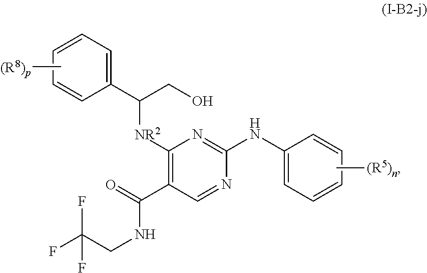

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-j*:

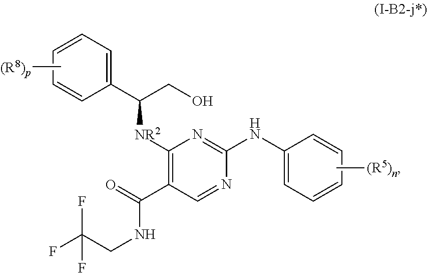

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B2-k:

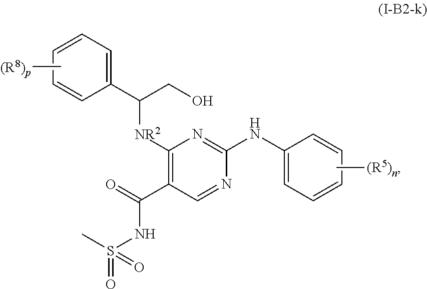

thereof, wherein p is an integer selected from 0, 1, 2, 3, 4 and 5 and all other variables are as defined herein.

In some embodiments, the present disclosure is related to compounds of Formula I-B2-k*:

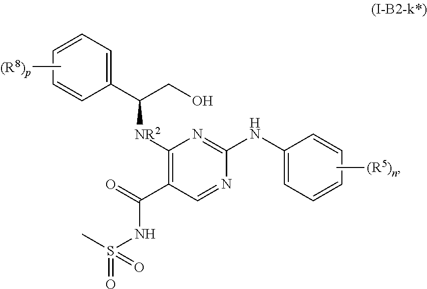

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-G1:

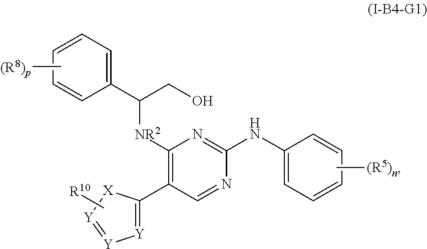

or pharmaceutically acceptable salts, solvates, prodrugs, enantiomers, stereoisomers, or tautomers thereof wherein, X is selected from NH, O, S; each of Y is independently selected from N, CH; R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-G2:

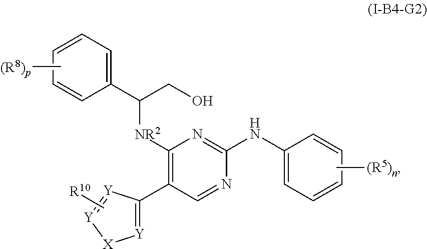

or pharmaceutically acceptable salts, solvates, prodrugs, enantiomers, stereoisomers, or tautomers thereof wherein, X is selected from NH, O, S; each of Y is independently selected from N, CH; R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-I*:

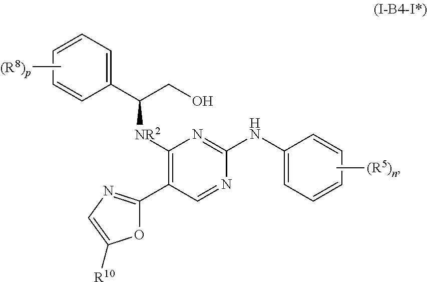

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-I:

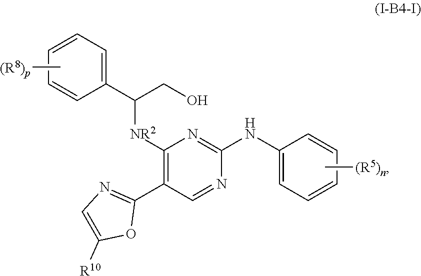

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-I*:

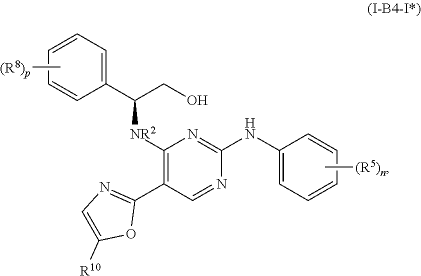

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-I-a:

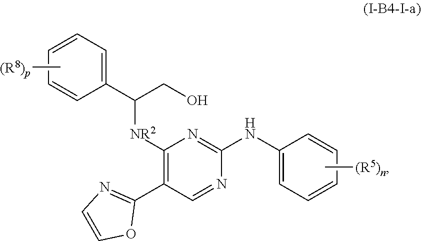

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-I-a*:

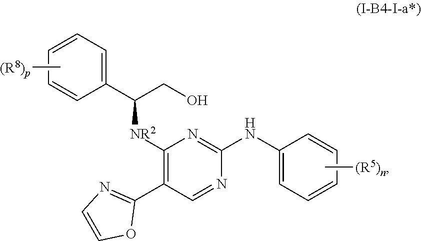

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-I-b:

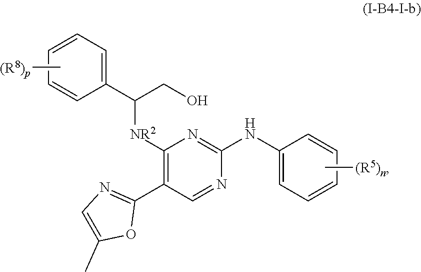

thereof, wherein p is an integer selected from 0, 1, 2, 3, 4 and 5 and all other variables are as defined herein.

In some embodiments, the present disclosure is related to compounds of Formula I-B4-I-b*:

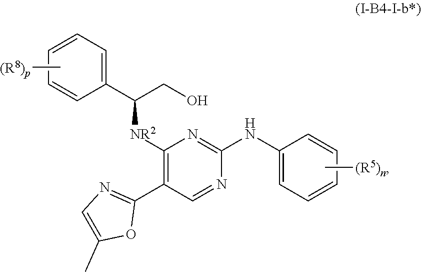

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II:

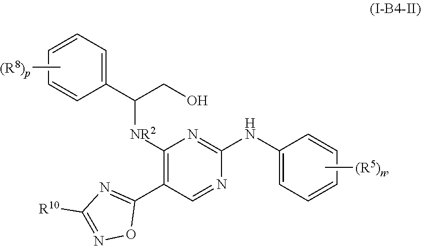

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-II*:

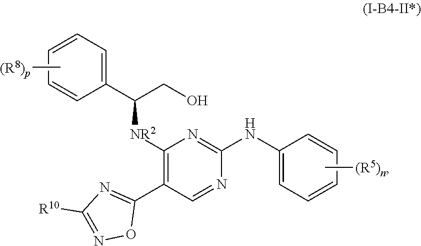

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-II-a:

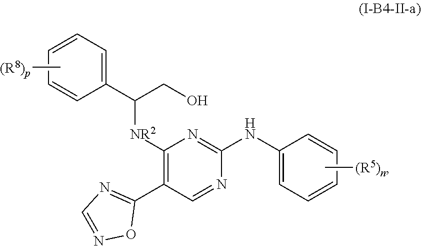

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-a*:

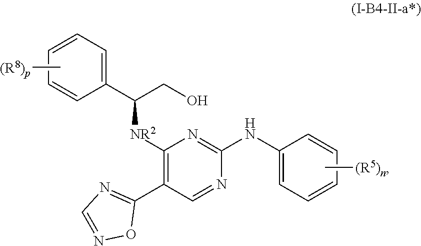

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-b:

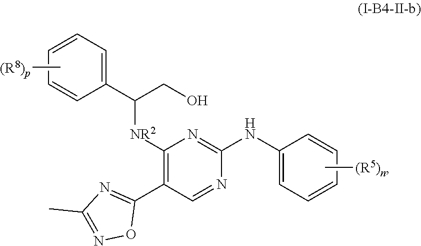

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-b*:

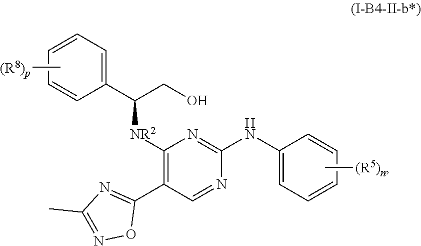

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-c:

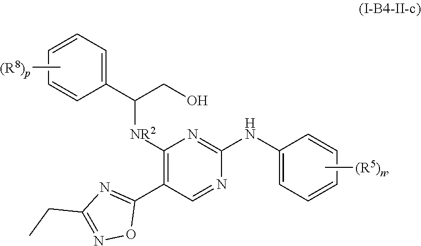

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-c*:

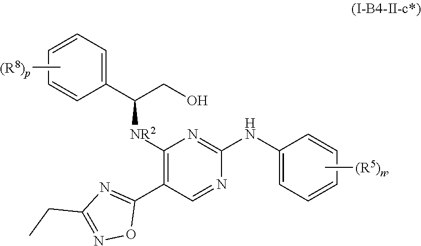

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-d:

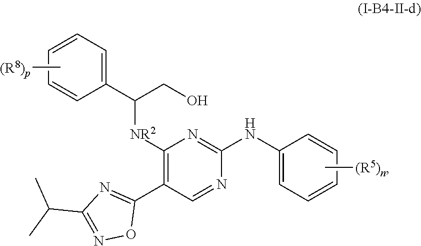

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-d*:

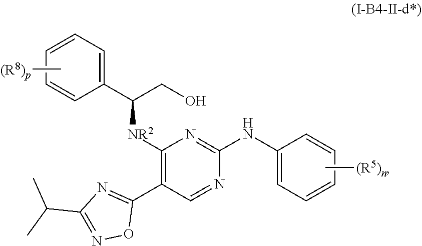

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-e:

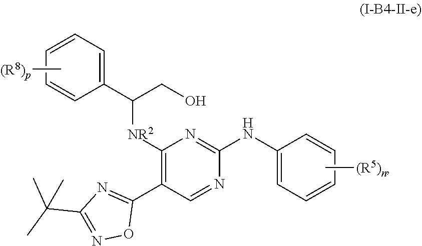

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-e*:

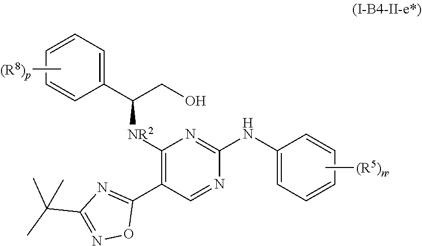

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-f:

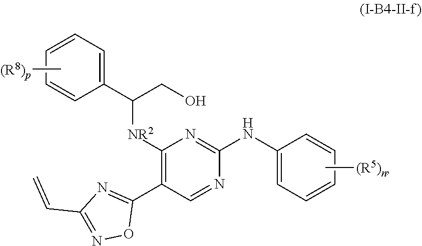

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-f*:

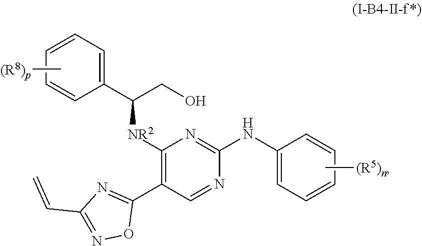

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-g:

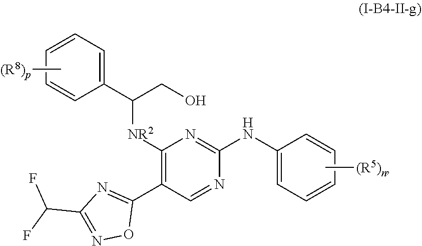

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-g*:

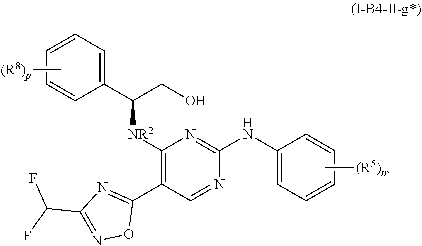

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-h:

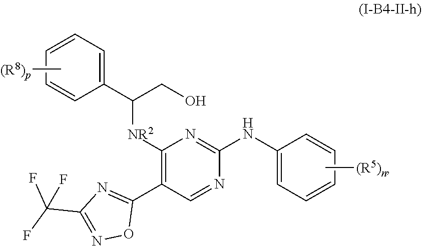

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-h*:

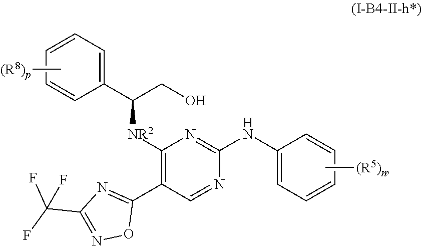

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-i:

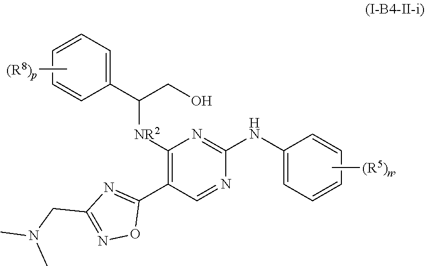

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-i*:

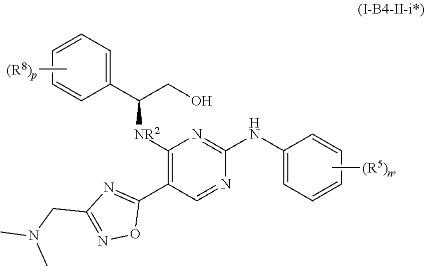

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-j:

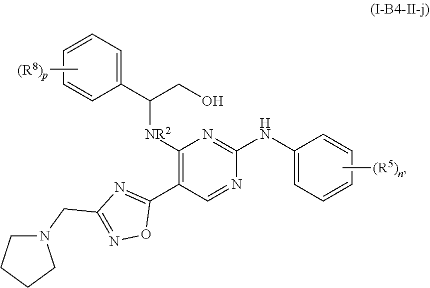

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-II-j*:

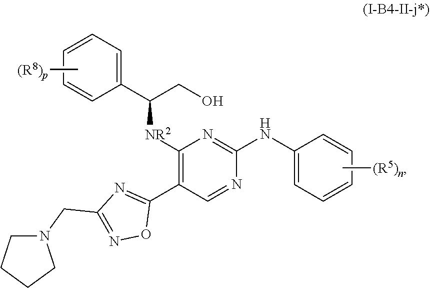

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III:

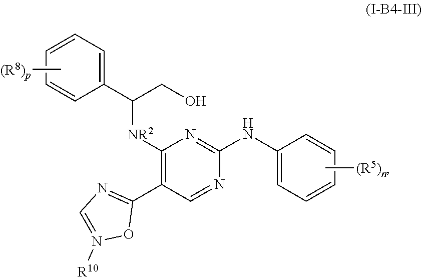

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-III*:

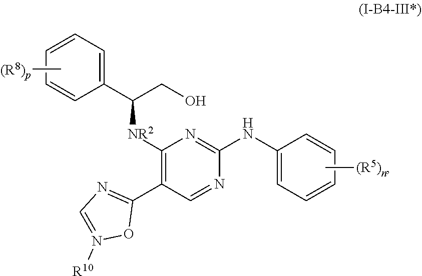

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-III-a:

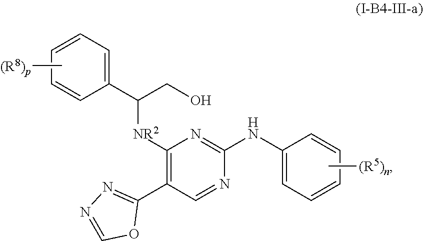

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-a*:

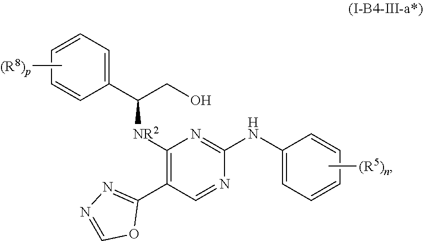

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-b:

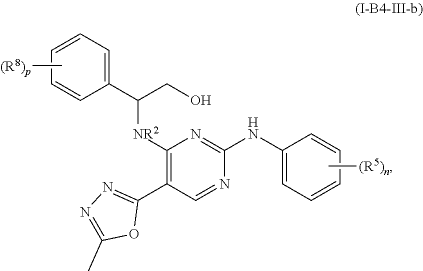

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-b*:

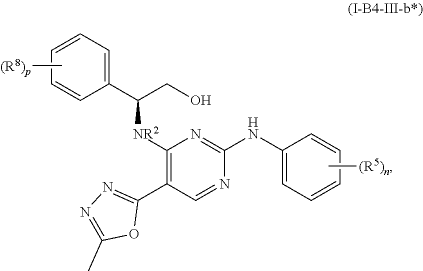

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-c:

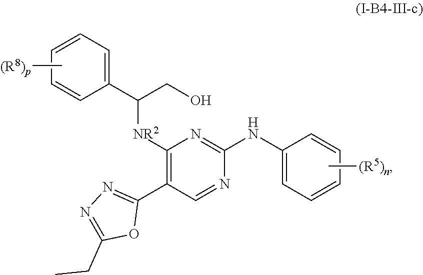

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-c*:

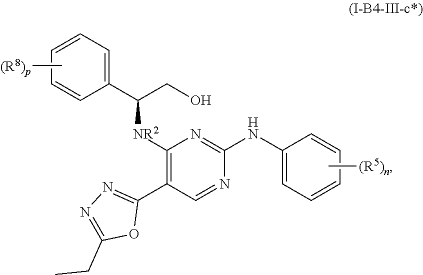

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-d:

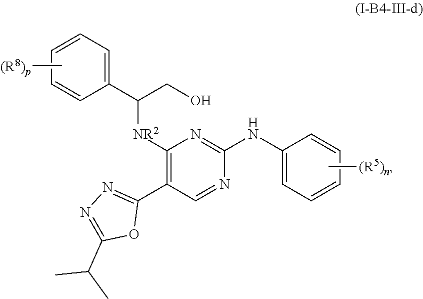

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-d*:

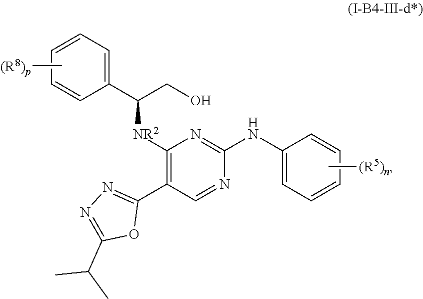

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-e:

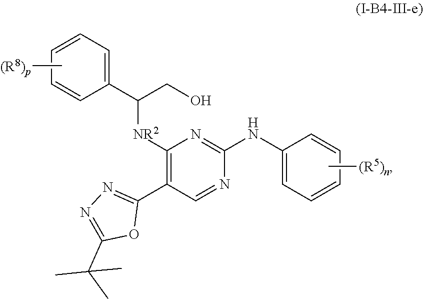

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-e*:

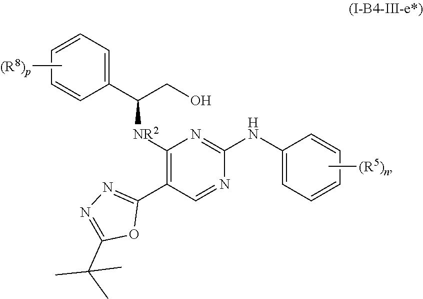

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-f:

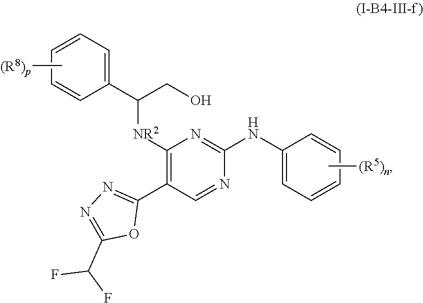

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-f*:

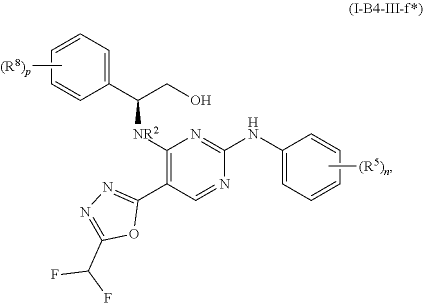

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-g:

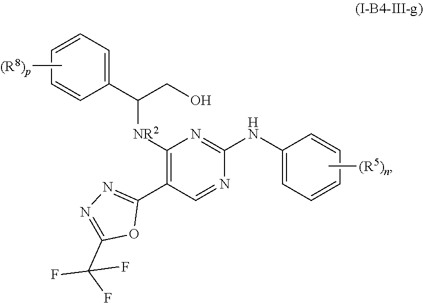

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-g*:

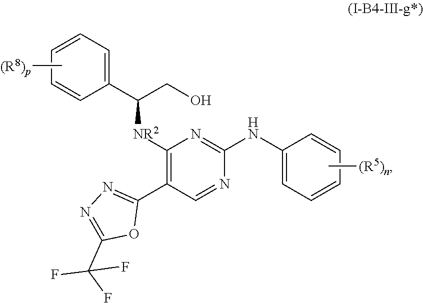

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-h:

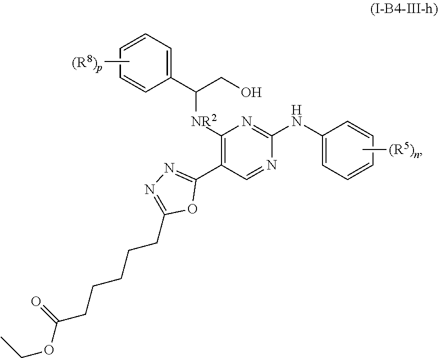

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-III-h*:

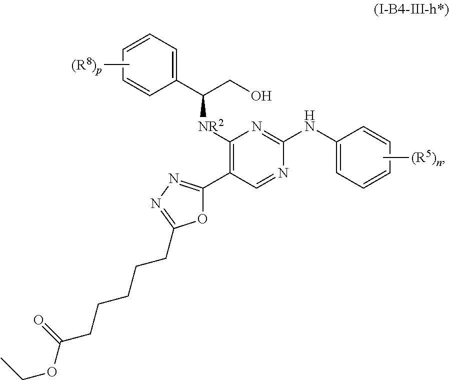

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-IV:

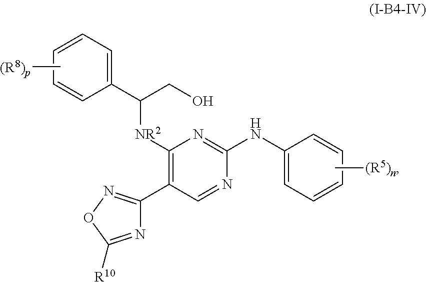

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-IV*:

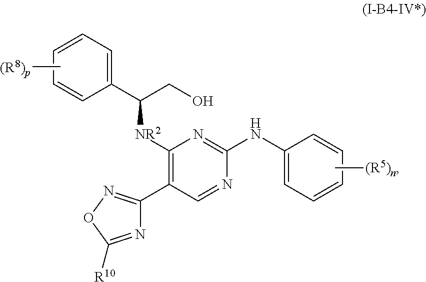

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-IV-a:

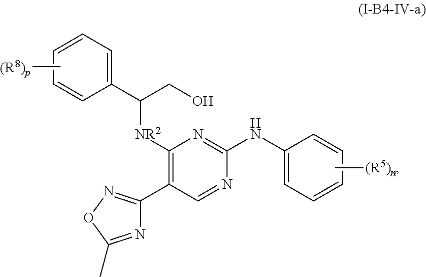

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-IV-a*:

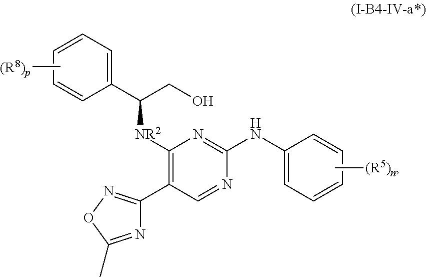

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-V:

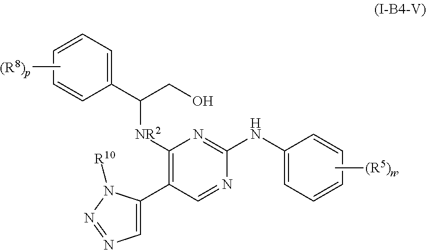

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-V*:

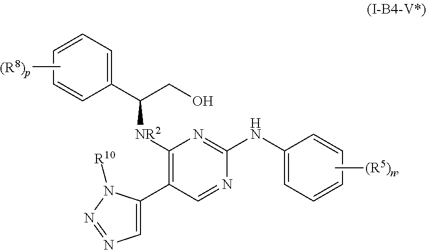

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-V-a:

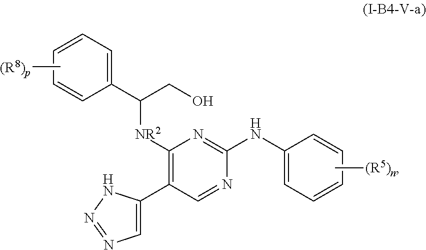

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-V-a*:

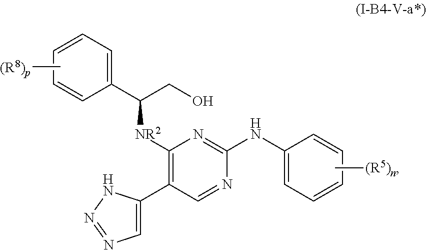

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VI:

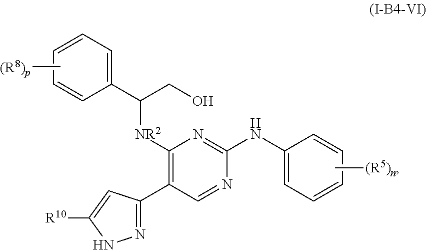

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-VI*:

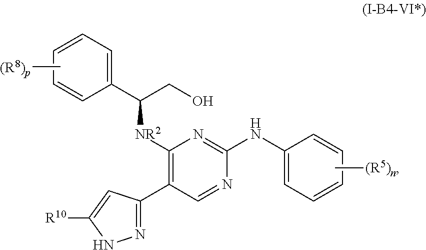

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-VI-a:

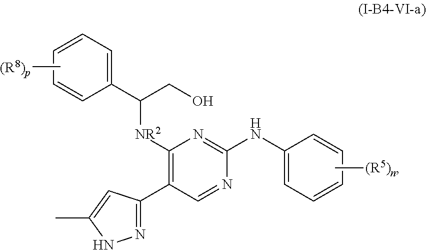

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VI-a*:

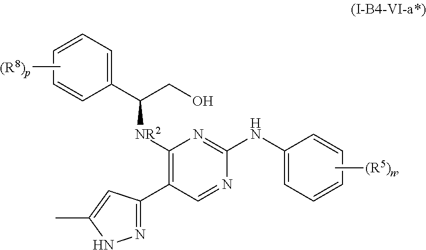

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VII:

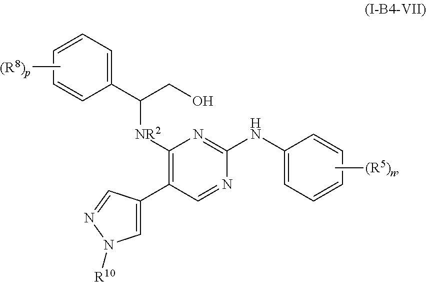

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-VII*:

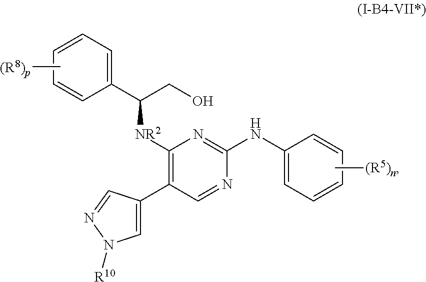

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-VII-a:

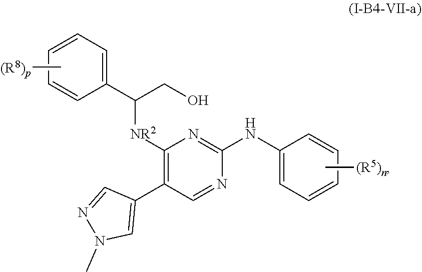

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VII-a*:

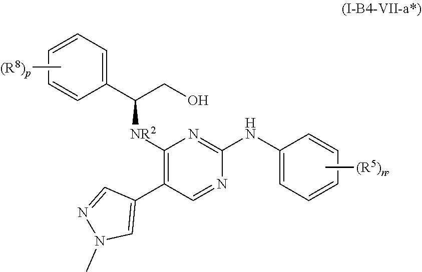

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VIII:

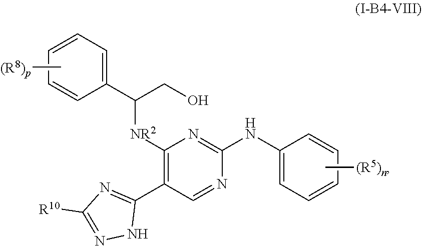

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-VIII*:

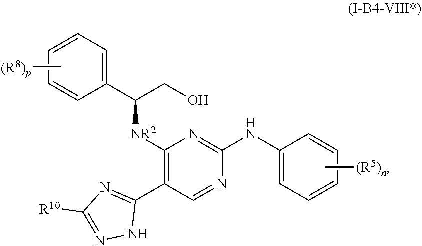

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-VIII-a:

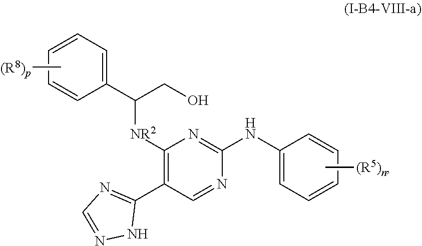

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VIII-a*:

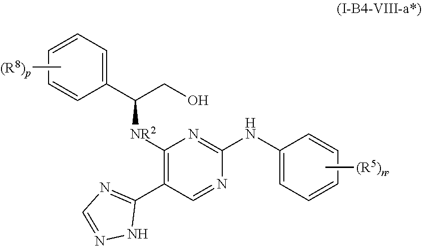

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VIII-b:

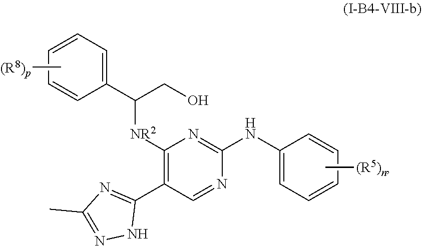

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VIII-b*:

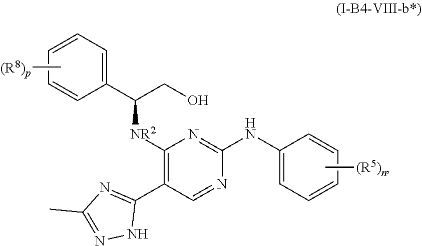

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VIII-c:

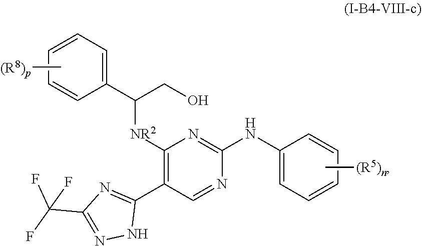

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-VIII-c*:

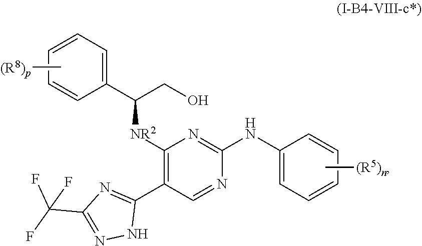

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-IX:

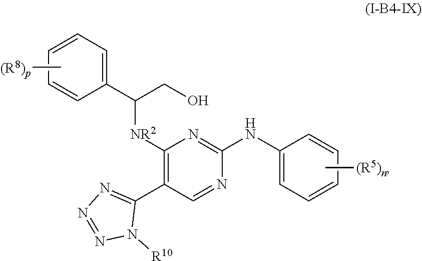

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-IX*:

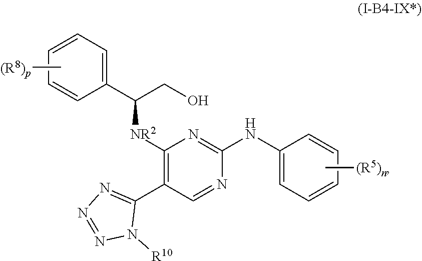

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-IX-a:

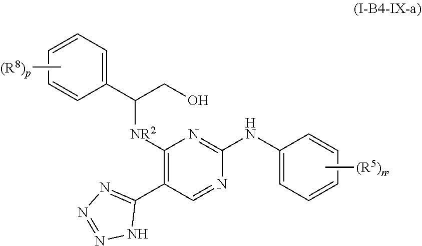

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-IX-a*:

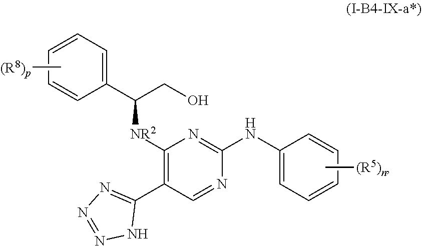

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-IX-b:

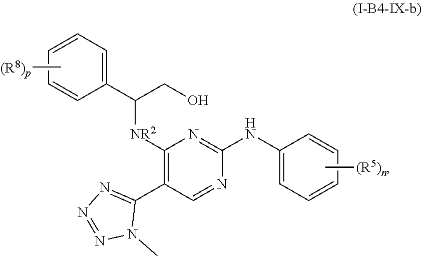

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-IX-b*:

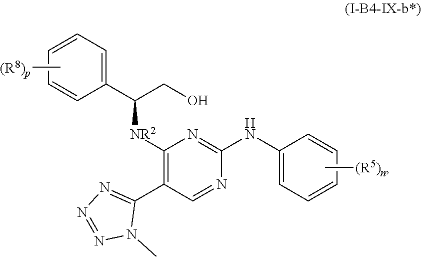

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-X:

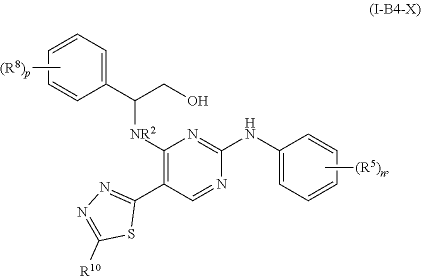

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-X*:

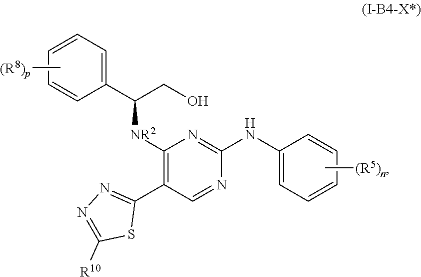

or pharmaceutically acceptable salts, solvates, prodrugs, enantiomers, stereoisomers, or tautomers thereof wherein R¹⁰ is selected from H, C₁₋₄ alkoxy, C₁₋₆ alkyl, C₂₋₆ alkenyl, 4-7 membered monocyclic heterocycloalkyl wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl, wherein p is an integer selected from 0, 1, 2, 3, 4 and 5 and all other variables are as defined herein.

In some embodiments, the present disclosure is related to compounds of Formula I-B4-X-a:

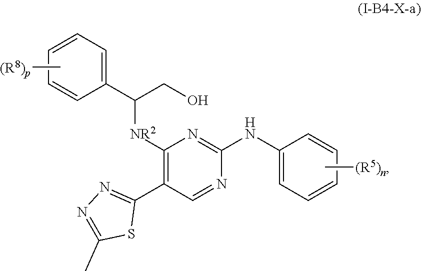

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-B4-X-a*:

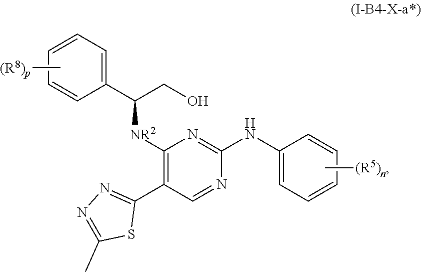

or pharmaceutically acceptable salts, solvates, prodrugs, enantiomers, stereoisomers, or tautomers thereof, wherein p is an integer selected from 0, 1, 2, 3, 4 and 5 and all other variables are as defined herein.

In some embodiments, the present disclosure is related to compounds of Formula I-C:

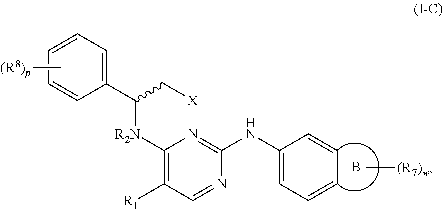

or pharmaceutically acceptable salts, solvates, prodrugs, enantiomers, stereoisomers, and tautomers thereof, wherein Ring B is 6-7 membered heterocyclyl containing at least one heteroatom selected from N, O, S, wherein w is an integer selected from 0, 1, 2 and 3, and all other variables are as defined herein.

In some embodiments, the present disclosure is related to compounds of Formula I-C*:

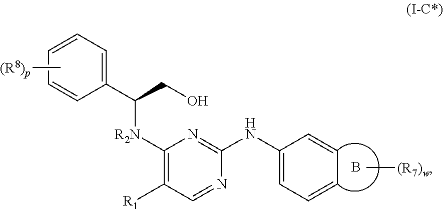

or pharmaceutically acceptable salts, solvates, prodrugs, enantiomers, stereoisomers, and tautomers thereof, wherein Ring B is 6-7 membered heterocyclyl containing at least one heteroatom selected from N, O, S, wherein w is an integer selected from 0, 1, 2 and 3, and all other variables are as defined herein.

In some embodiments, the present disclosure is related to compounds of Formula I-D:

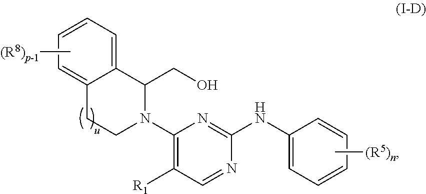

or pharmaceutically acceptable salts, solvates, prodrugs, enantiomers, stereoisomers, and tautomers thereof, wherein u is an integer selected from 0 or 1, and p is an integer from 1, 2, 3, 4 and 5 and all other variables are as defined herein.

In some embodiments, the present disclosure is related to compounds of Formula I-E:

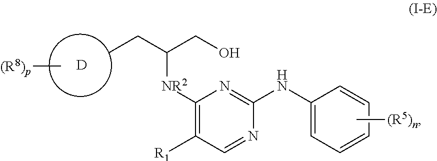

or pharmaceutically acceptable salts, solvates, prodrugs, enantiomers, stereoisomers, and tautomers thereof, wherein Ring D is aryl or heteroaryl; p is an integer from 0, 1, 2, 3, 4 and 5 and all other variables are as defined herein.

In some embodiments, the present disclosure is related to compounds of Formula I-F:

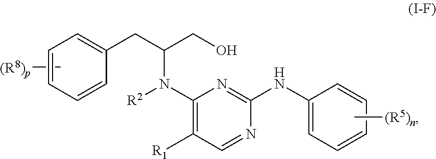

or pharmaceutically acceptable salts, solvates, prodrugs, enantiomers, stereoisomers, and tautomers thereof, wherein p is an integer selected from 0, 1, 2, 3, 4 and 5 and all other variables are as defined herein.

In some embodiments, the compounds of the instant disclosure are of Formula I-G

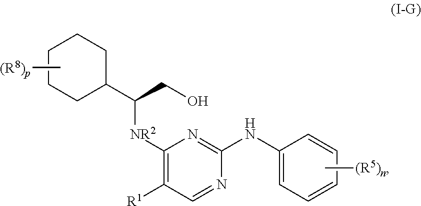

and pharmaceutically acceptable salts, solvates, prodrugs, enantiomers, stereoisomers, and tautomers thereof, wherein p is an integer selected from 0, 1, 2, 3, 4 and 5 and all other variables are as defined herein.

In some embodiments, the present disclosure is related to compounds of Formula I-H:

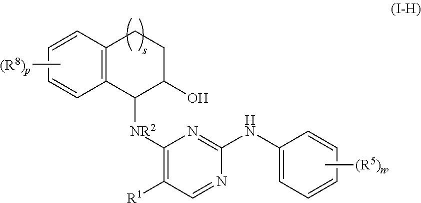

and pharmaceutically acceptable salts, solvates, prodrugs, enantiomers, stereoisomers, and tautomers thereof, wherein n is an integer selected from 0, 1, 2, 3, 4, and 5, and p is an integer selected from 0, 1, 2, 3, or 4, and s is 0 or 1 and all other variables are as defined herein.

In some embodiments, the present disclosure is related to compounds of Formula II-A:

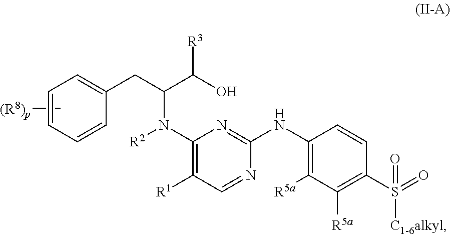

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-A*:

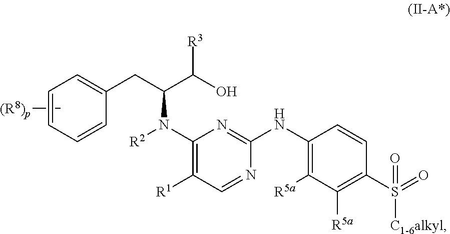

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-B:

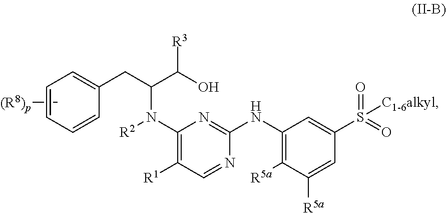

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-B*:

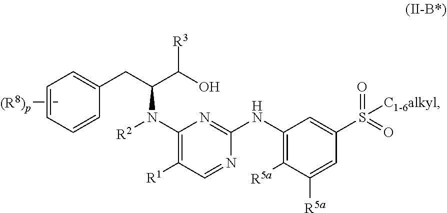

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-C:

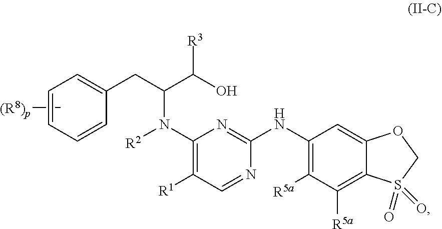

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-C*:

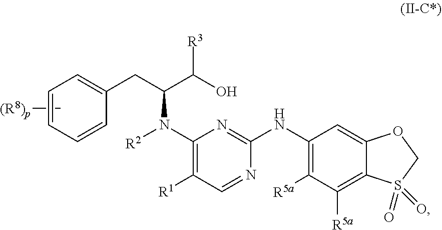

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-D:

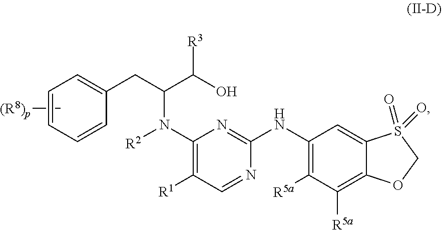

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-D*:

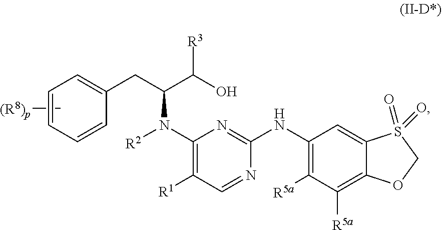

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-E:

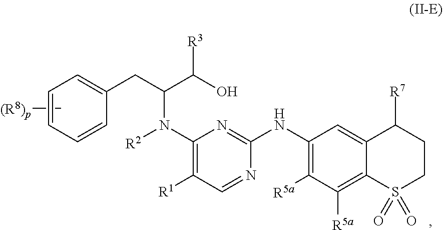

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-E*:

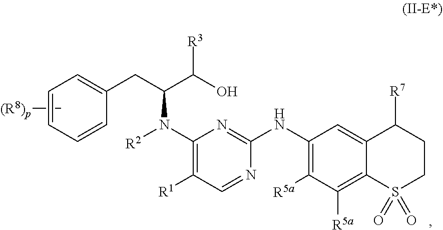

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-F:

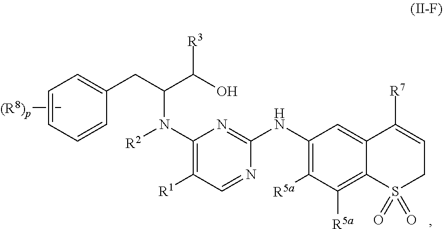

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-F*:

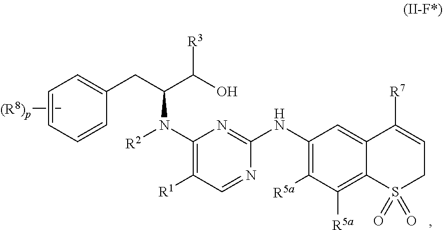

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-G:

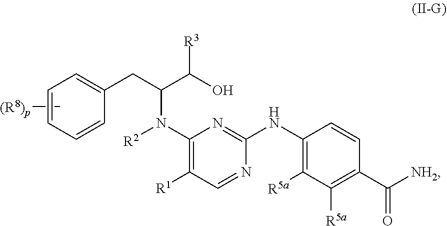

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-G*:

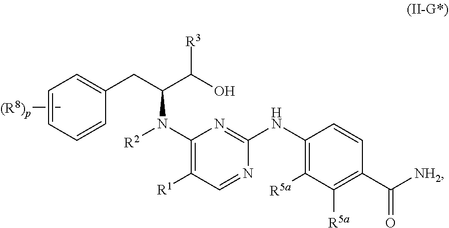

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-H:

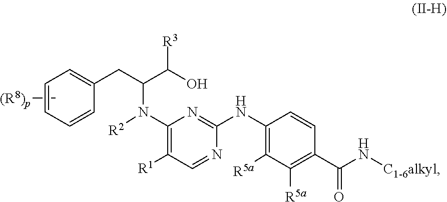

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-H*:

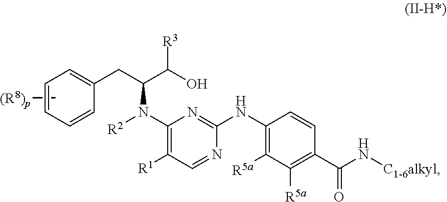

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-I:

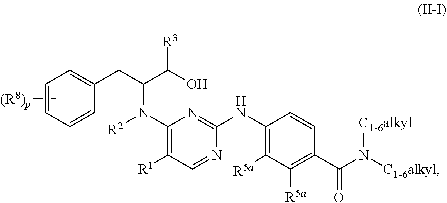

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-I*:

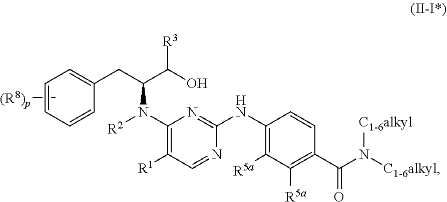

or pharmaceutically acceptable salts, solvates, prodrugs, enantiomers, stereoisomers, and tautomers thereof, wherein each R^{5a} is independently selected from H, halogen, C₁₋₆ alkyl, C₁₋₆ alkoxy, wherein p is an integer selected from 0, 1, 2, 3, 4 and 5, and all other variables are as defined herein.

In some embodiments, the present disclosure is related to compounds of Formula II-J-1, Formula II-J-2, Formula II-J-3, Formula II-J-4:

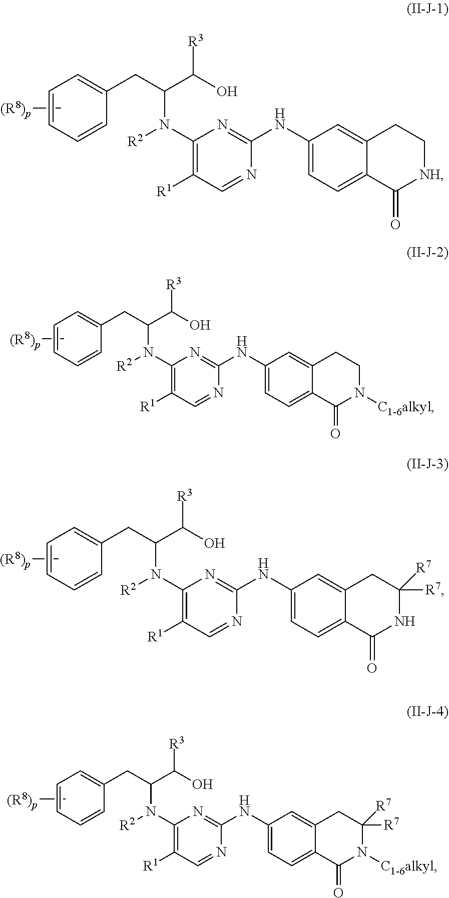

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-J-1*, Formula II-J-2*, Formula II-J-3*, Formula II-J-4*:

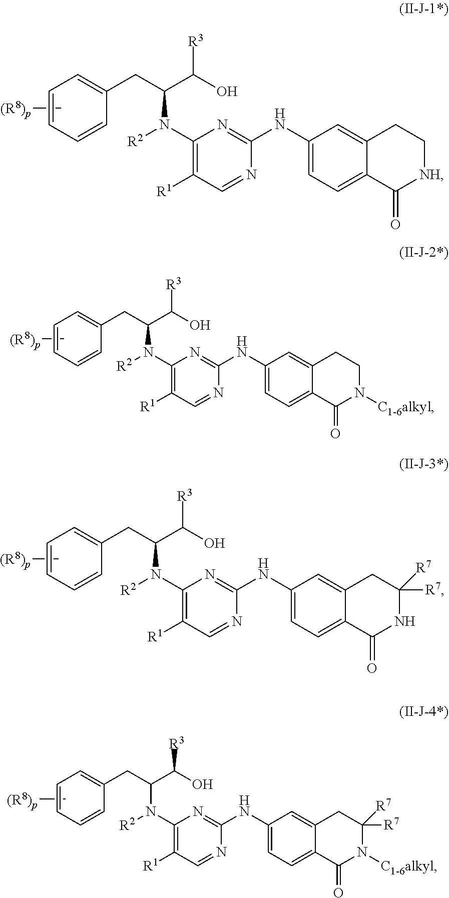

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-K:

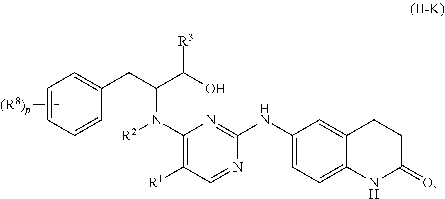

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-K*:

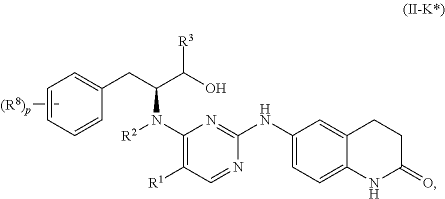

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-L-1, Formula II-L-2:

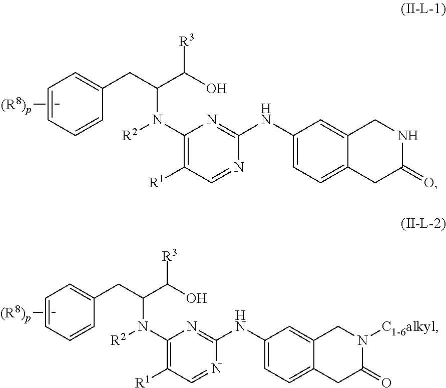

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-L-1*, Formula II-L-2*:

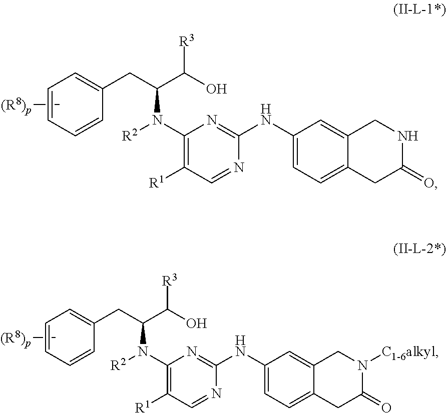

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-M-1, Formula II-M-2:

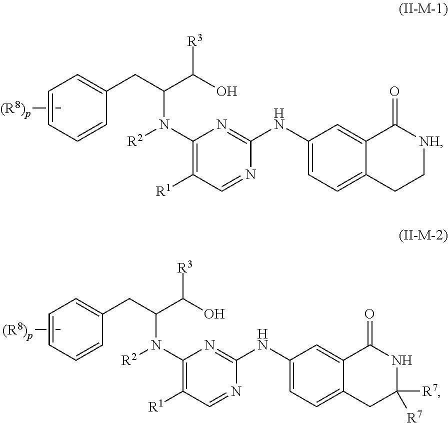

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-M-1*, Formula II-M-2*:

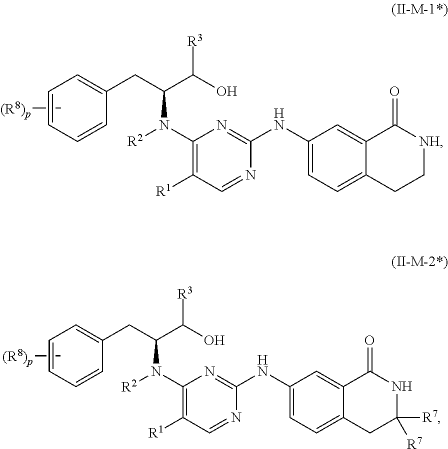

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-N:

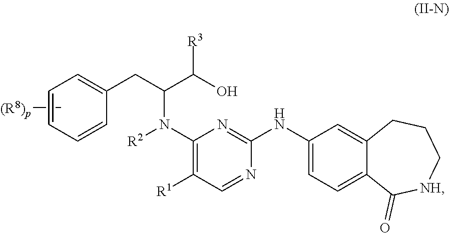

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-N*:

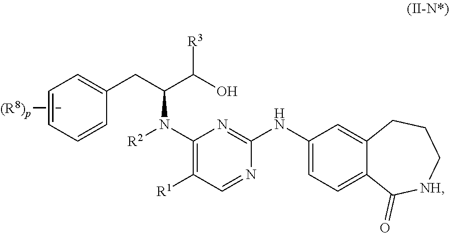

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-O:

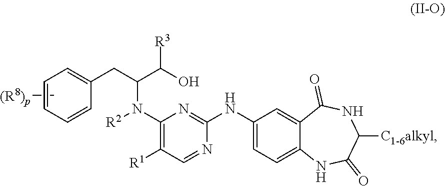

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-O*:

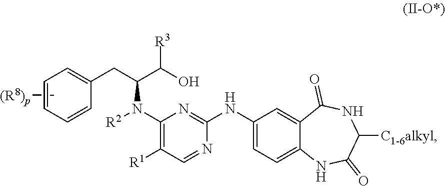

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-P:

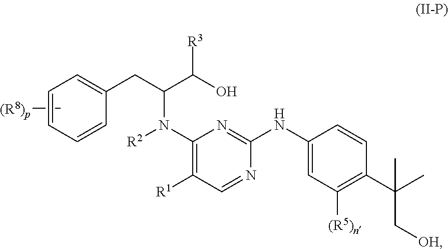

or pharmaceutically acceptable salts, solvates, prodrugs, enantiomers, stereoisomers, and tautomers thereof wherein n′ is selected from 0 and 1, wherein p is an integer selected from 0, 1, 2, 3, 4 and 5, and all other variables are as defined herein, and all other variables are as defined herein.

In some embodiments, the present disclosure is related to compounds of Formula II-P*:

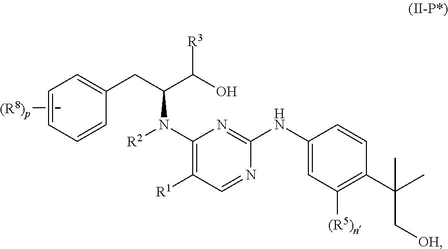

or pharmaceutically acceptable salts, solvates, prodrugs, enantiomers, stereoisomers, and tautomers thereof wherein n′ is selected from 0 and 1, wherein p is an integer selected from 0, 1, 2, 3, 4 and 5, and all other variables are as defined herein.

In some embodiments, the present disclosure is related to compounds of Formula II-R:

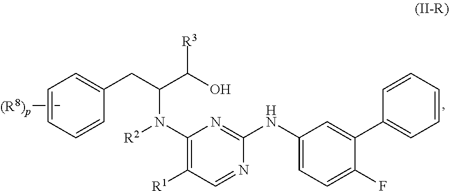

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-R*:

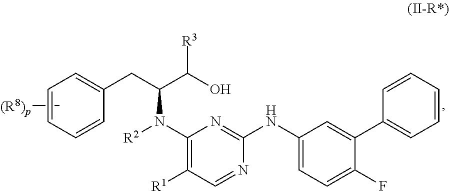

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-S:

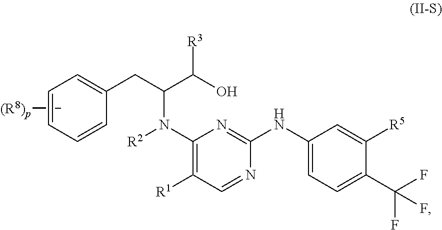

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, the present disclosure is related to compounds of Formula II-S*:

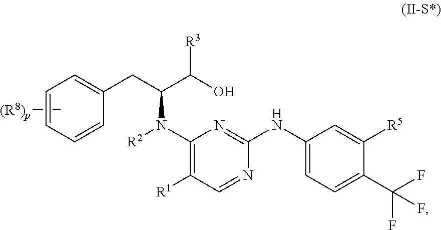

or pharmaceutically acceptable salts, solvates, prodrugs, enantiomers, stereoisomers, and tautomers thereof, wherein p is an integer selected from 0, 1, 2, 3, 4 and 5, and all other variables are as defined herein.

In some embodiments, X is H, halogen or OH. In some embodiments, X is halogen or OH. In some embodiments, X is H or halogen. In some embodiments, X is H or OH. In some embodiments, X is H. In some embodiments, X is halogen. In some embodiments, X is OH.

In some embodiments, R¹ is selected from —CN, —NO₂, —C(O)NHR₆, —C(O)N(R₆)₂, —C(O)OR₆, or a monocyclic heteroaryl comprising one or more heteroatoms independently selected from N, S, and O, wherein the heteroaryl is optionally substituted with one or more substituents selected from —OH, oxo, halogen, C₁₋₄ alkoxy, C₁₋₆ alkyl, C₂-C₆ alkenyl, or 4-7 membered monocyclic heterocycloalkyl, wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl.

In some embodiments, R¹ is selected from —CN, —NO₂, —C(O)NHR₆, C(O)N(R₆)₂, —C(O)OR₆.

In some embodiments, R¹ is monocyclic heteroaryl comprising one or more heteroatoms independently selected from N, S, and O, wherein the heteroaryl is optionally substituted with one or more substituents selected from —OH, oxo, halogen, C₁₋₄ alkoxy, C₁₋₆ alkyl, C₂-C₆ alkenyl, or 4-7 membered monocyclic heterocycloalkyl, wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl.

In some embodiments, R¹ is —CN.

In some embodiments, R¹ is —NO₂.

In some embodiments, R¹ is —C(O)NHR₆.

In some embodiments, R¹ is —C(O)N(R₆)₂.

In some embodiments, R¹ is —C(O)OR₆.

In some embodiments, R¹ is monocyclic heteroaryl comprising one or more heteroatoms independently selected from N, S, and O, wherein the heteroaryl is optionally substituted with one or more substituents selected from —OH, oxo, halogen, C₁₋₄ alkoxy, C₁₋₆ alkyl, C₂-C₆ alkenyl, or 4-7 membered monocyclic heterocycloalkyl, wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl.

In some embodiments, R¹ is selected from the table below

| | | | |
|---|---|---|---|
| —CN | —NO₂ | —C(O)OH | —C(O)OC₂H₅ |
| —C(O)NH₂ | —C(O)NHCH₃ | —C(O)NHOH | —C(O)N(CH₃)₂ |
| —C(O)NHCH₂CH₃ | —C(O)NHCH(CH₃)₂ | —C(O)OCH₂CH₂OH | —C(O)OCH₂CH₂OCH₃ |
| 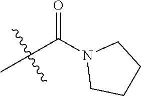 [SMILES: *C(=O)N1CCCC1] | 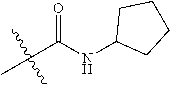 [SMILES: *C(=O)NC1CCCC1] | 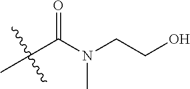 [SMILES: *C(=O)N(C)CCO] | 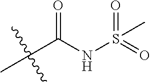 [SMILES: *C(=O)NS(C)(=O)=O] |
| 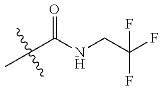 [SMILES: *C(=O)NCC(F)(F)F] | 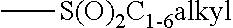 | 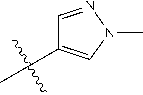 [SMILES: *c1cnn(C)c1] | 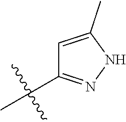 [SMILES: *c1cc(C)[nH]n1] |
| 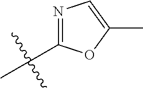 [SMILES: *c1ncc(C)o1] | 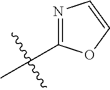 [SMILES: *c1ncco1] | 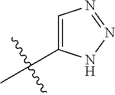 [SMILES: *c1cnn[nH]1] | 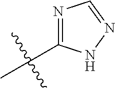 [SMILES: *c1ncn[nH]1] |
| 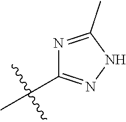 [SMILES: *c1n[nH]c(C)n1] | 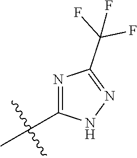 [SMILES: *c1nc(C(F)(F)F)n[nH]1] | 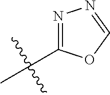 [SMILES: *c1nnco1] | 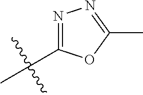 [SMILES: *c1nnc(C)o1] |
| 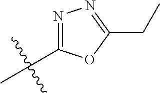 [SMILES: *c1nnc(CC)o1] | 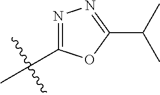 [SMILES: *c1nnc(C(C)C)o1] | 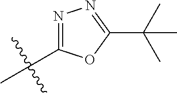 [SMILES: *c1nnc(C(C)(C)C)o1] | 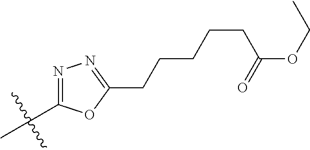 [SMILES: *c1nnc(CCCCCC(=O)OCC)o1] |
| 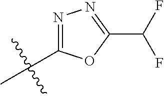 [SMILES: *c1nnc(C(F)F)o1] | 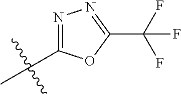 [SMILES: *c1nnc(C(F)(F)F)o1] | 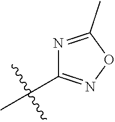 [SMILES: *c1noc(C)n1] | 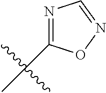 [SMILES: *c1ncno1] |
| 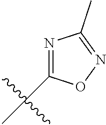 [SMILES: *c1nc(C)no1] | 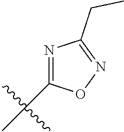 [SMILES: *c1nc(CC)no1] | 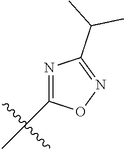 [SMILES: *c1nc(C(C)C)no1] | 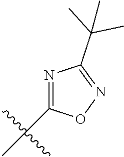 [SMILES: *c1nc(C(C)(C)C)no1] |
| 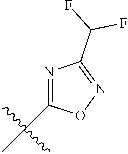 [SMILES: *c1nc(C(F)F)no1] | 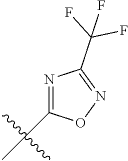 [SMILES: *c1nc(C(F)(F)F)no1] | 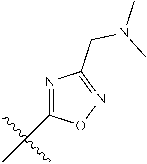 [SMILES: *c1nc(CN(C)C)no1] | 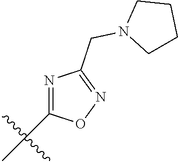 [SMILES: *c1nc(CN2CCCC2)no1] |
| 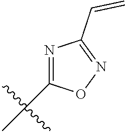 [SMILES: *c1nc(C=C)no1] | 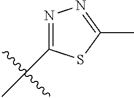 [SMILES: *c1nnc(C)s1] | 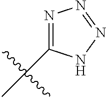 [SMILES: *c1nnn[nH]1] | 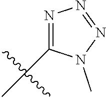 [SMILES: *c1nnnn1C] |

In some embodiments, R² is H or C₁₋₄ alkyl. In some embodiments, R² is H. In some embodiments, R² is C₁₋₄ alkyl.

In some embodiments, R² and R⁸ together with the atoms to which they are attached and any intervening atoms, form a 5- to-6-membered heterocycle.

In some embodiments, the present disclosure is related to compounds of Formula I-A-A:

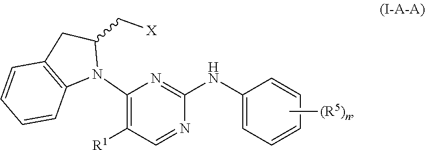

or pharmaceutically acceptable salts, solvates, prodrugs, enantiomers, stereoisomers, and tautomers thereof.

In some embodiments, the present disclosure is related to compounds of Formula I-A-A-1:

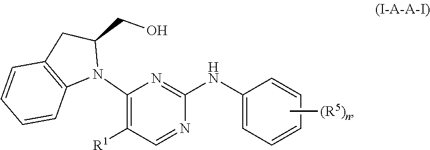

or pharmaceutically acceptable salts, solvates, prodrugs, enantiomers, stereoisomers, and tautomers thereof.

In some embodiments, the present disclosure is related to compounds of Formula I-A-B:

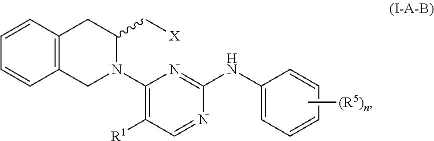

or pharmaceutically acceptable salts, solvates, prodrugs, enantiomers, stereoisomers, and tautomers thereof.

In some embodiments, the present disclosure is related to compounds of Formula I-A-B-1:

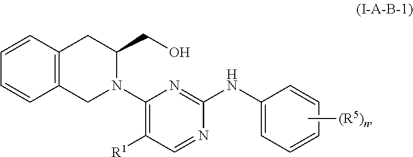

or pharmaceutically acceptable salts, solvates, prodrugs, enantiomers, stereoisomers, and tautomers thereof.

In some embodiments, R³ is H.

In some embodiments, R³ and R⁸ together with the atoms to which they are attached and any intervening atoms, form a 5- to-6-membered cycloalkyl.

In some embodiments, the present disclosure is related to compounds of Formula I-A-C:

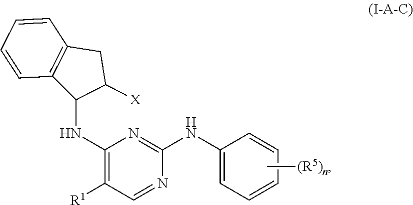

or pharmaceutically acceptable salts, solvates, prodrugs, enantiomers, stereoisomers, and tautomers thereof.

In some embodiments, the present disclosure is related to compounds of Formula I-A-C-1:

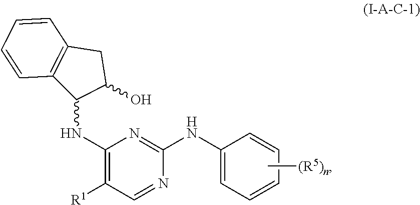

or pharmaceutically acceptable salts, solvates, prodrugs, enantiomers, stereoisomers, and tautomers thereof.

In some embodiments, R⁴ is —(CH₂)ₘ-aryl, —(CH₂)ₘ-heteroaryl, or heterocyclyl. In some embodiments, R⁴ is —(CH₂)ₘ-aryl, or —(CH₂)ₘ-heteroaryl. In another embodiment, R⁴ is —(CH₂)ₘ-aryl. In another embodiment, R⁴ is —(CH₂)ₘ-heteroaryl. In another embodiment, R⁴ is heterocyclyl.

In other embodiments, R⁴ is —(CH₂)ₘ-aryl, wherein the aryl is optionally substituted with one or more halogen, OH, or NH₂. In other embodiments, R⁴ is —(CH₂)ₘ-heteroaryl, wherein the heteroaryl is optionally substituted with one or more halogen, OH, or NH₂. In other embodiments, R⁴ is heterocyclyl, wherein the heterocyclyl is optionally substituted with one or more halogen, OH, or NH₂.

In some embodiments, each R⁵ is independently selected from halogen, C₁₋₆ alkyl, C₁₋₆ alkoxy, C₁₋₆ halogenalkyl, oxy C₁₋₆ alkyl, —S(O)₂—C₁₋₆ alkyl, —S(O)₂—C₂₋₆ alkenyl, —C(O)NH₂, —C(O)NH(C₁₋₆ alkyl), —C(O)N(C₁₋₆ alkyl)₂, —CH₂C(O)NH₂, —CH₂C(O)NH(C₁₋₆ alkyl), —CH₂C(O)N(C₁₋₆ alkyl)₂, —NHC(O)CH₃, aryl, heteroaryl.

In some embodiments, R⁵ is halogen. In some embodiments, R⁵ is F. In some embodiments, R⁵ is Cl.

In some embodiments, R⁵ is C₁₋₆ alkyl.

In some embodiments, R⁵ is —CH₃.

In some embodiments, R⁵ is C₁₋₆ alkoxy.

In some embodiments, R⁵ is —OCH₃.

In some embodiments, R⁵ is C₁₋₆ halogenalkyl.

In some embodiments, R⁵ is

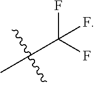 [SMILES: *C(F)(F)F]

In other embodiments, R⁵ is oxy C₁₋₆ alkyl.

In other embodiments, R⁵ is

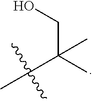 [SMILES: *C(C)(C)CO]

In some embodiments, R⁵ is —S(O)₂—C₁₋₆ alkyl.

In some embodiments, R⁵ is

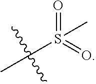 [SMILES: *S(C)(=O)=O]

In some embodiments, R⁵ is

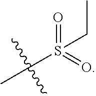 [SMILES: *S(=O)(=O)CC]

In some embodiments, R⁵ is

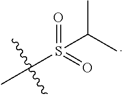 [SMILES: *S(=O)(=O)C(C)C]

In some embodiments, R⁵ is —C(O)NH₂.

In some embodiments, R⁵ is —C(O)NH(C₁₋₆ alkyl).

In some embodiments, R⁵ is

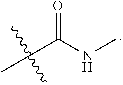 [SMILES: *C(=O)NC]

In some embodiments, R⁵ is —C(O)N(C₁₋₆ alkyl)₂.

In some embodiments, R⁵ is

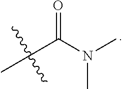 [SMILES: *C(=O)N(C)C]

In some embodiments, R⁵ is aryl.

In some embodiments, R⁵ is phenyl.

In other embodiments, at least one R⁵ is halogen or —S(O)₂—C₁-C₆ alkyl.

In other embodiments, at least one R⁵ is halogen and another R⁵ is —S(O)₂—CH₃.

In other embodiments, at least one R⁵ is methyl and another R⁵ is —S(O)₂—CH₃.

In some embodiments, two R⁵ together with the atoms to which they are attached and any intervening atoms, form 5-7 membered heterocyclyl, or 5-7 membered heteroaryl, containing at least one heteroatom selected from N, O, S; wherein the cycloalkyl or heterocyclyl is optionally substituted with one or more R⁷.

In some embodiments, the fragment

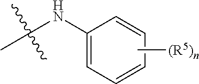

of compound of Formula (I) is selected from the table below

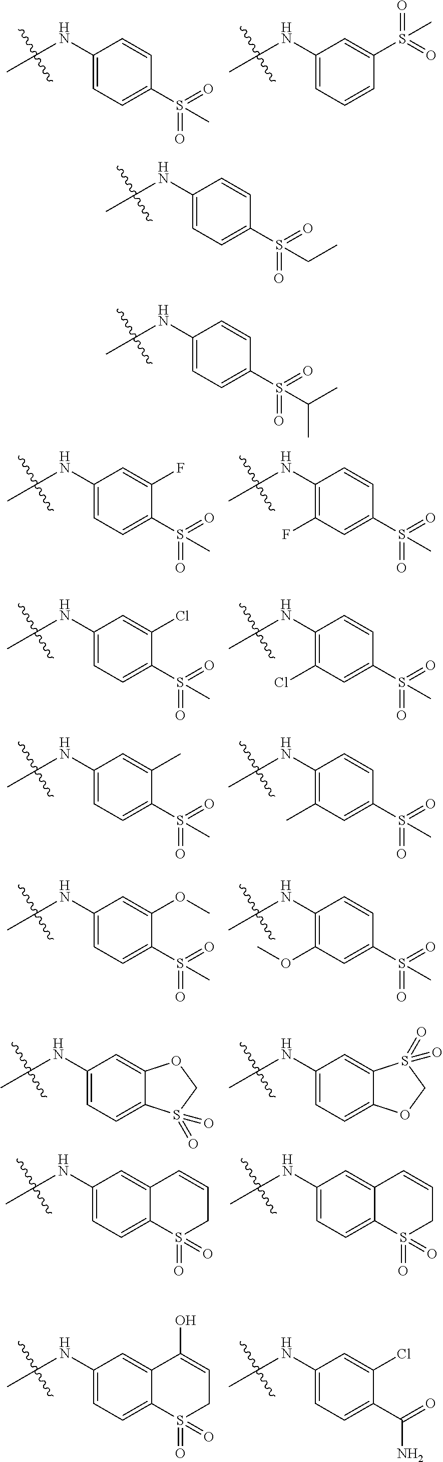

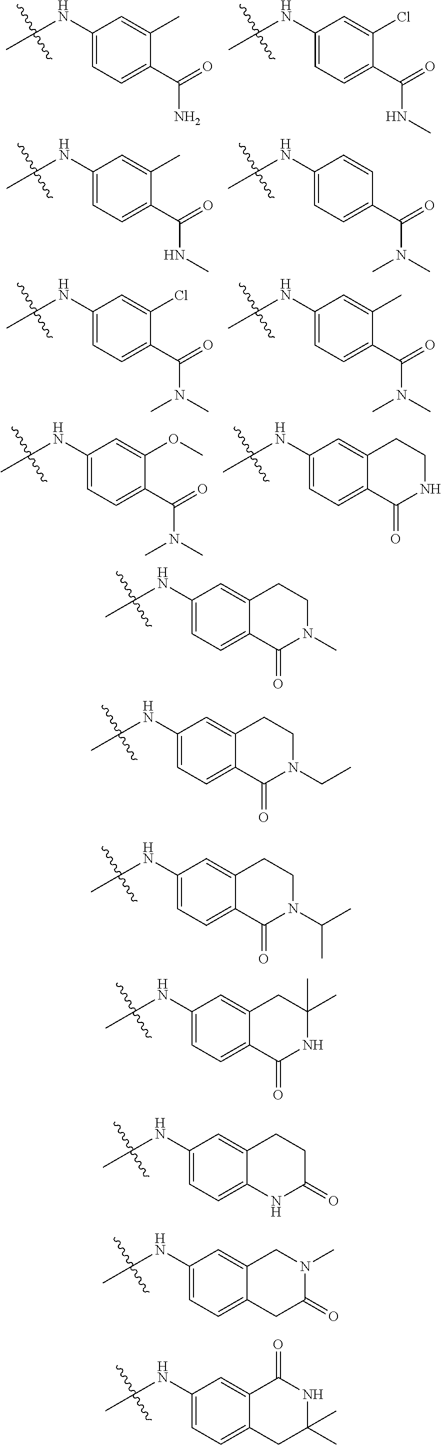

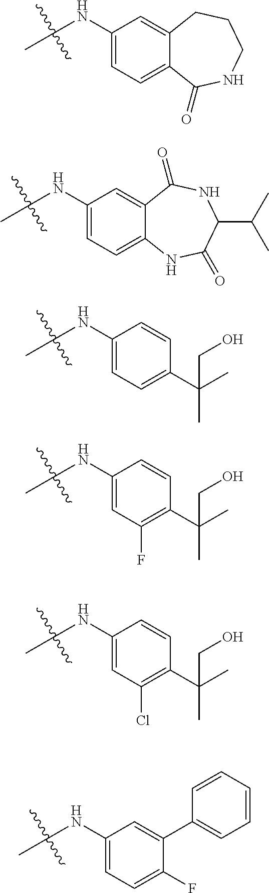

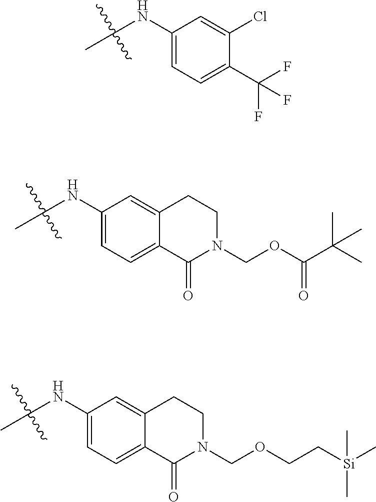 [SMILES: *Nc1ccc(C(F)(F)F)c(Cl)c1 ; *Nc1ccc2c(c1)CCN(COC(=O)C(C)(C)C)C2=O ; *Nc1ccc2c(c1)CCN(COCC[Si](C)(C)C)C2=O]

In some embodiments, R⁶ is independently selected from H, OH, C₁₋₆ alkyl, —S(O)₂—C₁₋₆ alkyl, C₃₋₈ cycloalkyl, heterocyclyl, or heteroaryl, wherein the alkyl, alkoxy, heteroaryl, or heterocyclyl is optionally substituted with one or more R⁹.

In some embodiments, R⁶ is H.

In some embodiments, R⁶ is OH.

In some embodiments, R⁶ is C₁₋₆ alkyl.

In other embodiments, R⁷ is oxo or C₁₋₆ alkyl. In other embodiments, R⁷ is oxo. In other embodiments, R⁷ is C₁₋₆ alkyl.

In some embodiments, R⁸ is halogen, OH, or NH₂. In some embodiments, R⁸ is halogen. In some embodiments, R⁸ is OH. In some embodiments, R⁸ is NH₂.

In other embodiments, R⁹ is halogen, OH, NH₂, C₁₋₆ alkyl, C₁₋₆ alkoxy, C₁₋₆ haloalkyl, C₂₋₆ alkenyl, C₂₋₆ alkynyl, C₁₋₆ haloalkyl, C₃₋₈ cycloalkyl, heterocyclyl, aryl, or heteroaryl. In some embodiments, R⁹ is halogen. In some embodiments, R⁹ is OH. In some embodiments, R⁹ is NH₂. In some embodiments, R⁹ is C₁₋₆ alkyl. In some embodiments, R⁹ is C₁₋₆ alkoxy. In some embodiments, R⁹ is C₁₋₆ haloalkyl. In some embodiments, R⁹ is C₂₋₆ alkenyl. In some embodiments, R⁹ is C₂₋₆ alkynyl. In some embodiments, R⁹ is C₁₋₆ haloalkyl. In some embodiments, R⁹ is C₃₋₈ cycloalkyl. In some embodiments, R⁹ is heterocyclyl. In some embodiments, R⁹ is aryl. In some embodiments, R⁹ is or heteroaryl.

In some embodiments, m is 0, 1, 2, 3, 4, 5, or 6. In some embodiments, m is 0, 1, 2, 3, 4, or 5. In some embodiments, m is 0, 1, 2, 3, or 4. In some embodiments, m is 0, 1, 2, or 3. In some embodiments, m is 0, 1, or 2. In some embodiments, m is 0 or 1. In some embodiments, m is 0. In some embodiments, m is 1. In some embodiments, m is 2. In some embodiments, m is 3. In some embodiments, m is 4. In some embodiments, m is 5. In some embodiments, m is 6.

In some embodiments, n is 0, 1, 2, 3, 4, 5, or 6. In some embodiments, n is 0, 1, 2, 3, 4, or 5. In some embodiments, n is 0, 1, 2, 3, or 4. In some embodiments, n is 0, 1, 2, or 3. In some embodiments, n is 0, 1, or 2. In some embodiments, n is 0 or 1. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, p is 1, 2, 3, 4, or 5. In some embodiments, p is 1, 2, 3, or 4. In some embodiments, p is 1, 2, or 3. In some embodiments, p is 1 or 2. In some embodiments, p is 1. In some embodiments, p is 2. In some embodiments, p is 3. In some embodiments, p is 4. In some embodiments, p is 5

In some embodiments, t is 1, 2, 3, or 4. In some embodiments, t is 1, 2, or 3. In some embodiments, t is 1 or 2. In some embodiments, t is 1. In some embodiments, t is 2. In some embodiments, t is 3. In some embodiments, t is 4.

In some embodiments, s is 0 or 1. In some embodiments, s is 0. In some embodiments, s is 1.

In some embodiments, u is 0 or 1. In some embodiments, u is 0. In some embodiments, u is 1.

In other embodiments, Ring A represents a monocyclic heteroaryl containing at last one nitrogen atom.

In some embodiments, Ring D is aryl or heteroaryl. In some embodiments, Ring D is aryl. In some embodiments, Ring D is heteroaryl.

Non-limiting illustrative compounds of the present disclosure include:
4-[[(1S)-2-hydroxy-1-phenyl-ethyl]amino]-2-(3-methyl-4-methylsulfonylanilino)-pyrimidine-5-carbonitrile;
2-(3-fluoro-4-methylsulfonyl-anilino)-4-[[(1S)-2-hydroxy-1-phenylethyl]amino]-pyrimidine-5-carbonitrile;
4-[[4-[[(1S)-2-hydroxy-1-phenyl-ethyl]amino]-5-oxazol-2-yl-pyrimidin-2-yl]amino]-2-methyl-benzamide;
4-[[4-[[(1S)-2-hydroxy-1-phenyl-ethyl]amino]-5-(1H-1,2,4-triazol-5-yl)pyrimidin-2-yl]-amino]-2-methyl-benzamide;
4-[[4-[[(1S)-2-hydroxy-1-phenyl-ethyl]amino]-5-(1H-triazol-5-yl)pyrimidin-2-yl]amino]-2-methyl-benzamide;
4-[[4-[[(1S)-2-hydroxy-1-phenyl-ethyl]amino]-5-(1,2,4-oxadiazol-5-yl)pyrimidin-2-yl]-amino]-2-methyl-benzamide;
4-[[4-[[(1S)-2-hydroxy-1-phenyl-ethyl]amino]-5-(1,3,4-oxadiazol-2-yl)pyrimidin-2-yl]-amino]-2-methyl-benzamide;
4-[[4-[[(1S)-2-hydroxy-1-phenyl-ethyl]amino]-5-(1H-tetrazol-5-yl)pyrimidin-2-yl]-amino]-2-methyl-benzamide;
4-[[(1S)-2-hydroxy-1-phenyl-ethyl]amino]-N-methyl-2-[(1-oxo-3,4-dihydro-2H-isoquinolin-6-yl)amino]pyrimidine-5-carboxamide;
ethyl 2-(4-carbamoyl-3-methyl-anilino)-4-[[(1S)-2-hydroxy-1-phenyl-ethyl]amino]-pyrimidine-5-carboxylate;
2-[4-(2-hydroxy-1,1-dimethyl-ethyl)anilino]-4-[[(1S)-2-hydroxy-1-phenyl-ethyl]amino]-N-methyl-pyrimidine-5-carboxamide;
4-[[(1S)-2-hydroxy-1-phenyl-ethyl]amino]-2-(3-methyl-4-methylsulfonyl-anilino)-pyrimidine-5-carboxamide;
6-[[4-[[(1S)-2-hydroxy-1-phenyl-ethyl]amino]-5-oxazol-2-yl-pyrimidin-2-yl]amino]-3,4-dihydro-2H-isoquinolin-1-one;
6-[[4-[[(1S)-2-hydroxy-1-phenyl-ethyl]amino]-5-oxazol-2-yl-pyrimidin-2-yl]amino]-3,4-dihydro-1H-quinolin-2-one;
6-[[4-[[(1S)-2-hydroxy-1-phenyl-ethyl]amino]-5-(1H-triazol-5-yl)pyrimidin-2-yl]amino]-3,4-dihydro-2H-isoquinolin-1-one;
6-[[4-[[(1S)-2-hydroxy-1-phenyl-ethyl]amino]-5-(1H-triazol-5-yl)pyrimidin-2-yl]amino]-3,4-dihydro-1H-quinolin-2-one;
6-[[4-[[(1S)-2-hydroxy-1-phenyl-ethyl]amino]-5-(1H-1,2,4-triazol-5-yl)pyrimidin-2-yl]amino]-3,4-dihydro-1H-quinolin-2-one;
(2S)-2-[[2-(3-methyl-4-methylsulfonyl-anilino)-5-nitro-pyrimidin-4-yl]amino]-2-phenyl-ethanol;
6-[[4-[[(1S)-2-hydroxy-1-phenyl-ethyl]amino]-5-(1,3,4-oxadiazol-2-yl)pyrimidin-2-yl]amino]-3,4-dihydro-1H-quinolin-2-one;
6-[[4-[[(1S)-2-hydroxy-1-phenyl-ethyl]amino]-5-(1,2,4-oxadiazol-5-yl)pyrimidin-2-yl]amino]-3,4-dihydro-1H-quinolin-2-one;
6-[[4-[[(1S)-2-hydroxy-1-phenyl-ethyl]amino]-5-(1H-tetrazol-5-yl)pyrimidin-2-yl]amino]-3,4-dihydro-1H-quinolin-2-one;
4-[[4-[[(1S)-2-hydroxy-1-phenyl-ethyl]amino]-5-(5-methyl-1H-pyrazol-3-yl)pyrimidin-2-yl]amino]-2-methyl-benzamide;
4-[[4-[[(1S)-2-hydroxy-1-phenyl-ethyl]amino]-5-oxazol-2-yl-pyrimidin-2-yl]amino]-N,N-dimethyl-benzamide;
4-[[4-[[(1S)-2-hydroxy-1-phenyl-ethyl]amino]-5-oxazol-2-yl-pyrimidin-2-yl]amino]-N,2-dimethyl-benzamide;
4-[[4-[[(1S)-2-hydroxy-1-phenyl-ethyl]amino]-5-(5-methyloxazol-2-yl)pyrimidin-2-yl]-amino]-2-methyl-benzamide;
4-[[4-[[(1S)-2-hydroxy-1-phenyl-ethyl]amino]-5-(1H-triazol-5-yl)pyrimidin-2-yl]amino]-N,N-dimethyl-benzamide;
4-[[4-[[(1S)-2-hydroxy-1-phenyl-ethyl]amino]-5-(1H-triazol-5-yl)pyrimidin-2-yl]amino]-N,2-dimethyl-benzamide;
4-[[4-[[(1S)-2-hydroxy-1-phenyl-ethyl]amino]-5-(1H-1,2,4-triazol-5-yl)pyrimidin-2-yl]-amino]-N,N-dimethyl-benzamide;
4-[[4-[[(1S)-2-hydroxy-1-phenyl-ethyl]amino]-5-(1H-1,2,4-triazol-5-yl)pyrimidin-2-yl]-amino]-N,2-dimethyl-benzamide;
4-[[4-[[(1S)-2-hydroxy-1-phenyl-ethyl]amino]-5-(5-methyl-1H-1,2,4-triazol-3-yl)-pyrimidin-2-yl]amino]-2-methyl-benzamide;
2-(3-fluoro-4-methylsulfonyl-anilino)-4-[[(1S)-2-hydroxy-1-phenyl-ethyl]amino]-pyrimidine-5-carboxamide;
4-[[4-[[(1S)-2-hydroxy-1-phenyl-ethyl]amino]-5-(1,3,4-oxadiazol-2-yl)pyrimidin-2-yl]-amino]-N,N-dimethyl-benzamide;
4-[[4-[[(1S)-2-hydroxy-1-phenyl-ethyl]amino]-5-(1,3,4-oxadiazol-2-yl)pyrimidin-2-yl]-amino]-N,2-dimethyl-benzamide;
4-[[4-[[(1S)-2-hydroxy-1-phenyl-ethyl]amino]-5-(1,2,4-oxadiazol-5-yl)pyrimidin-2-yl]-amino]-N,N-dimethyl-benzamide;
4-[[4-[[(1S)-2-hydroxy-1-phenyl-ethyl]amino]-5-(1,2,4-oxadiazol-5-yl)pyrimidin-2-yl]-amino]-N,2-dimethyl-benzamide;
4-[[4-[[(1S)-2-hydroxy-1-phenyl-ethyl]amino]-5-(3-methyl-1,2,4-oxadiazol-5-yl)-pyrimidin-2-yl]amino]-2-methyl-benzamide;
4-[[4-[[(1S)-2-hydroxy-1-phenyl-ethyl]amino]-5-(5-methyl-1,3,4-oxadiazol-2-yl)-pyrimidin-2-yl]amino]-2-methyl-benzamide;
4-[[4-[[(1S)-2-hydroxy-1-phenyl-ethyl]amino]-5-(5-methyl-1,2,4-oxadiazol-3-yl)-pyrimidin-2-yl]amino]-2-methyl-benzamide;
4-[[4-[[(1S)-2-hydroxy-1-phenyl-ethyl]amino]-5-(1H-tetrazol-5-yl)pyrimidin-2-yl]-amino]-N,N-dimethyl-benzamide;
4-[[4-[[(1S)-2-hydroxy-1-phenyl-ethyl]amino]-5-(1H-tetrazol-5-yl)pyrimidin-2-yl]-amino]-N,2-dimethyl-benzamide;
4-[[4-[[(1S)-2-hydroxy-1-phenyl-ethyl]amino]-5-(1-methyltetrazol-5-yl)pyrimidin-2-yl]-amino]-2-methyl-benzamide;
2-(3-fluoro-4-methylsulfonyl-anilino)-4-[[(1S)-2-hydroxy-1-phenyl-ethyl]amino]-pyrimidine-5-carboxylic acid;
4-[[(1S)-2-hydroxy-1-phenyl-ethyl]amino]-N-methyl-2-[(2-methyl-1-oxo-3,4-dihydroisoquinolin-6-yl)amino]pyrimidine-5-carboxamide;
4-[[(1S)-2-hydroxy-1-phenyl-ethyl]amino]-N-methyl-2-[(2-methyl-3-oxo-1,4-dihydroisoquinolin-7-yl)amino]pyrimidine-5-carboxamide;
ethyl 4-[[(1S)-2-hydroxy-1-phenyl-ethyl]amino]-2-[(1-oxo-3,4-dihydro-2H-isoquinolin-6-yl)amino]pyrimidine-5-carboxylate;
ethyl 4-[[(1S)-2-hydroxy-1-phenyl-ethyl]amino]-2-[(2-oxo-3,4-dihydro-1H-quinolin-6-yl)amino]pyrimidine-5-carboxylate;
ethyl 2-[4-(dimethylcarbamoyl)anilino]-4-[[(1S)-2-hydroxy-1-phenyl-ethyl]amino]-pyrimidine-5-carboxylate;
ethyl 4-[[(1S)-2-hydroxy-1-phenyl-ethyl]amino]-2-[3-methyl-4-(methylcarbamoyl)-anilino]pyrimidine-5-carboxylate;
2-chloro-4-[[4-[[(1S)-2-hydroxy-1-phenyl-ethyl]amino]-5-oxazol-2-yl-pyrimidin-2-yl]-amino]benzamide;
2-chloro-4-[[4-[[(1S)-2-hydroxy-1-phenyl-ethyl]amino]-5-(1H-1,2,4-triazol-5-yl)-pyrimidin-2-yl]amino]benzamide;
2-chloro-4-[[4-[[(1S)-2-hydroxy-1-phenyl-ethyl]amino]-5-(1H-triazol-5-yl)pyrimidin-2-yl]amino]benzamide;
ethyl 2-[4-(2-hydroxy-1,1-dimethyl-ethyl)anilino]-4-[[(1S)-2-hydroxy-1-phenyl-ethyl]amino]pyrimidine-5-carboxylate;
2-chloro-4-[[4-[[(1S)-2-hydroxy-1-phenyl-ethyl]amino]-5-(1,2,4-oxadiazol-5-yl)pyrimidin-2-yl]amino]benzamide;
2-chloro-4-[[4-[[(1S)-2-hydroxy-1-phenyl-ethyl]amino]-5-(1,3,4-oxadiazol-2-yl)pyrimidin-2-yl]amino]benzamide;
2-chloro-4-[[4-[[(1S)-2-hydroxy-1-phenyl-ethyl]amino]-5-(1H-tetrazol-5-yl)pyrimidin-2-yl]amino]benzamide;
(2S)-2-[[2-(4-methylsulfonylanilino)-5-oxazol-2-yl-pyrimidin-4-yl]amino]-2-phenyl-ethanol;
(2S)-2-[[2-(4-methylsulfonylanilino)-5-(1H-1,2,4-triazol-5-yl)pyrimidin-4-yl]amino]-2-phenyl-ethanol;
(2S)-2-[[2-(4-methylsulfonylanilino)-5-(1H-triazol-5-yl)pyrimidin-4-yl]amino]-2-phenyl-ethanol;
(2S)-2-[[2-(4-methylsulfonylanilino)-5-(1,2,4-oxadiazol-5-yl)pyrimidin-4-yl]amino]-2-phenyl-ethanol;
(2S)-2-[[2-(4-methylsulfonylanilino)-5-(1,3,4-oxadiazol-2-yl)pyrimidin-4-yl]amino]-2-phenyl-ethanol;
(2S)-2-[[2-(4-methylsulfonylanilino)-5-(1H-tetrazol-5-yl)pyrimidin-4-yl]amino]-2-phenyl-ethanol;
4-[[5-ethoxycarbonyl-4-[[(1S)-2-hydroxy-1-phenyl-ethyl]amino]pyrimidin-2-yl]amino]-2-methoxy-benzoic acid;
2-[3-fluoro-4-(2-hydroxy-1,1-dimethyl-ethyl)anilino]-4-[[(1S)-2-hydroxy-1-phenyl-ethyl]amino]-N-methyl-pyrimidine-5-carboxamide;
ethyl 2-(3-methyl-4-methylsulfonyl-anilino)-4-[[(1R)-1-phenylethyl]amino]pyrimidine-5-carboxylate;
4-[[(1S)-2-hydroxy-1-phenyl-ethyl]amino]-N-methyl-2-(3-methyl-4-methylsulfonyl-anilino)pyrimidine-5-carboxamide;
6-[[4-[[(1S)-2-hydroxy-1-phenyl-ethyl]amino]-5-(5-methyl-1H-pyrazol-3-yl)pyrimidin-2-yl]amino]-3,4-dihydro-2H-isoquinolin-1-one;
6-[[4-[[(1S)-2-hydroxy-1-phenyl-ethyl]amino]-5-(5-methyl-1H-pyrazol-3-yl)pyrimidin-2-yl]amino]-3,4-dihydro-1H-quinolin-2-one;
ethyl 4-[[(1S)-2-hydroxy-1-phenyl-ethyl]amino]-2-(4-methylsulfonylanilino)pyrimidine-5-carboxylate;
ethyl 4-[[(1S)-2-hydroxy-1-phenyl-ethyl]amino]-2-(3-methylsulfonylanilino)pyrimidine-5-carboxylate;
6-[[4-[[(1S)-2-hydroxy-1-phenyl-ethyl]amino]-5-(5-methyloxazol-2-yl)pyrimidin-2-yl]amino]-3,4-dihydro-2H-isoquinolin-1-one;
6-[[4-[[(1S)-2-hydroxy-1-phenyl-ethyl]amino]-5-(5-methyloxazol-2-yl)pyrimidin-2-yl]amino]-3,4-dihydro-1H-quinolin-2-one;
6-[[4-[[(1S)-2-hydroxy-1-phenyl-ethyl]amino]-5-oxazol-2-yl-pyrimidin-2-yl]amino]-2-methyl-3,4-dihydroisoquinolin-1-one;
7-[[4-[[(1S)-2-hydroxy-1-phenyl-ethyl]amino]-5-oxazol-2-yl-pyrimidin-2-yl]amino]-2-methyl-1,4-dihydroisoquinolin-3-one;
6-[[4-[[(1S)-2-hydroxy-1-phenyl-ethyl]amino]-5-(5-methyl-1H-1,2,4-triazol-3-yl)pyrimidin-2-yl]amino]-3,4-dihydro-2H-isoquinolin-1-one;
6-[[4-[[(1S)-2-hydroxy-1-phenyl-ethyl]amino]-5-(5-methyl-1H-1,2,4-triazol-3-yl)pyrimidin-2-yl]amino]-3,4-dihydro-1H-quinolin-2-one;
6-[[4-[[(1S)-2-hydroxy-1-phenyl-ethyl]amino]-5-(1H-triazol-5-yl)pyrimidin-2-yl]amino]-2-methyl-3,4-dihydroisoquinolin-1-one;
7-[[4-[[(1S)-2-hydroxy-1-phenyl-ethyl]amino]-5-(1H-triazol-5-yl)pyrimidin-2-yl]amino]-2-methyl-1,4-dihydroisoquinolin-3-one;
6-[[4-[[(1S)-2-hydroxy-1-phenyl-ethyl]amino]-5-(1H-1,2,4-triazol-5-yl)pyrimidin-2-yl]amino]-2-methyl-3,4-dihydroisoquinolin-1-one;
7-[[4-[[(1S)-2-hydroxy-1-phenyl-ethyl]amino]-5-(1H-1,2,4-triazol-5-yl)pyrimidin-2-yl]amino]-2-methyl-1,4-dihydroisoquinolin-3-one;
4-[[(1S)-2-hydroxy-1-phenyl-ethyl]amino]-2-(3-methyl-4-methylsulfonyl-anilino)pyrimidine-5-carbohydroxamic acid;
6-[[4-[[(1S)-2-hydroxy-1-phenyl-ethyl]amino]-5-(5-methyl-1,2,4-oxadiazol-3-yl)pyrimidin-2-yl]amino]-3,4-dihydro-2H-isoquinolin-1-one;
2-(4-fluoro-3-phenyl-anilino)-4-[[(1S)-2-hydroxy-1-phenyl-ethyl]amino]-N-methyl-pyrimidine-5-carboxamide;
6-[[4-[[(1S)-2-hydroxy-1-phenyl-ethyl]amino]-5-(5-methyl-1,2,4-oxadiazol-3-yl)pyrimidin-2-yl]amino]-3,4-dihydro-1H-quinolin-2-one;
6-[[4-[[(1S)-2-hydroxy-1-phenyl-ethyl]amino]-5-(5-methyl-1,3,4-oxadiazol-2-yl)pyrimidin-2-yl]amino]-3,4-dihydro-2H-isoquinolin-1-one;
6-[[4-[[(1S)-2-hydroxy-1-phenyl-ethyl]amino]-5-(5-methyl-1,3,4-oxadiazol-2-yl)pyrimidin-2-yl]amino]-3,4-dihydro-1H-quinolin-2-one;
6-[[4-[[(1S)-2-hydroxy-1-phenyl-ethyl]amino]-5-(3-methyl-1,2,4-oxadiazol-5-yl)pyrimidin-2-yl]amino]-3,4-dihydro-1H-quinolin-2-one;
6-[[4-[[(1S)-2-hydroxy-1-phenyl-ethyl]amino]-5-(1,2,4-oxadiazol-5-yl)pyrimidin-2-yl]amino]-2-methyl-3,4-dihydroisoquinolin-1-one;
7-[[4-[[(1S)-2-hydroxy-1-phenyl-ethyl]amino]-5-(1,2,4-oxadiazol-5-yl)pyrimidin-2-yl]amino]-2-methyl-1,4-dihydroisoquinolin-3-one;
6-[[4-[[(1S)-2-hydroxy-1-phenyl-ethyl]amino]-5-(1,3,4-oxadiazol-2-yl)pyrimidin-2-yl]amino]-2-methyl-3,4-dihydroisoquinolin-1-one;
7-[[4-[[(1S)-2-hydroxy-1-phenyl-ethyl]amino]-5-(1,3,4-oxadiazol-2-yl)pyrimidin-2-yl]amino]-2-methyl-1,4-dihydroisoquinolin-3-one;
6-[[4-[[(1S)-2-hydroxy-1-phenyl-ethyl]amino]-5-(1-methyltetrazol-5-yl)pyrimidin-2-yl]amino]-3,4-dihydro-2H-isoquinolin-1-one;
6-[[4-[[(1S)-2-hydroxy-1-phenyl-ethyl]amino]-5-(1-methyltetrazol-5-yl)pyrimidin-2-yl]amino]-3,4-dihydro-1H-quinolin-2-one;
6-[[4-[[(1S)-2-hydroxy-1-phenyl-ethyl]amino]-5-(1H-tetrazol-5-yl)pyrimidin-2-yl]amino]-2-methyl-3,4-dihydroisoquinolin-1-one;
7-[[4-[[(1S)-2-hydroxy-1-phenyl-ethyl]amino]-5-(1H-tetrazol-5-yl)pyrimidin-2-yl]amino]-2-methyl-1,4-dihydroisoquinolin-3-one;
4-[[4-[[(1S)-2-hydroxy-1-phenyl-ethyl]amino]-5-(5-methyl-1H-pyrazol-3-yl)pyrimidin-2-yl]amino]-N,N-dimethyl-benzamide;
4-[[4-[[(1S)-2-hydroxy-1-phenyl-ethyl]amino]-5-(5-methyl-1H-pyrazol-3-yl)pyrimidin-2-yl]amino]-N,2-dimethyl-benzamide;
ethyl 2-(3-fluoro-4-methylsulfonyl-anilino)-4-[[(1R)-1-phenylethyl]amino]pyrimidine-5-carboxylate;
4-[[4-[[(1S)-2-hydroxy-1-phenyl-ethyl]amino]-5-(5-methyloxazol-2-yl)pyrimidin-2-yl]amino]-N,N-dimethyl-benzamide;
4-[[4-[[(1S)-2-hydroxy-1-phenyl-ethyl]amino]-5-(5-methyloxazol-2-yl)pyrimidin-2-yl]amino]-N,2-dimethyl-benzamide;
4-[[4-[[(1S)-2-hydroxy-1-phenyl-ethyl]amino]-5-oxazol-2-yl-pyrimidin-2-yl]amino]-N,N,2-trimethyl-benzamide;
4-[[4-[[(1S)-2-hydroxy-1-phenyl-ethyl]amino]-5-(5-methyl-1H-1,2,4-triazol-3-yl)pyrimidin-2-yl]amino]-N,N-dimethyl-benzamide;
4-[[4-[[(1S)-2-hydroxy-1-phenyl-ethyl]amino]-5-(5-methyl-1H-1,2,4-triazol-3-yl)pyrimidin-2-yl]amino]-N,2-dimethyl-benzamide;
4-[[4-[[(1S)-2-hydroxy-1-phenyl-ethyl]amino]-5-(1H-triazol-5-yl)pyrimidin-2-yl]amino]-N,N,2-trimethyl-benzamide;
4-[[4-[[(1S)-2-hydroxy-1-phenyl-ethyl]amino]-5-(1H-1,2,4-triazol-5-yl)pyrimidin-2-yl]amino]-N,N,2-trimethyl-benzamide;
2-(3-fluoro-4-methylsulfonyl-anilino)-4-[[(1S)-2-hydroxy-1-phenyl-ethyl]amino]-N-methyl-pyrimidine-5-carboxamide;
4-[[4-[[(1S)-2-hydroxy-1-phenyl-ethyl]amino]-5-(5-methyl-1,2,4-oxadiazol-3-yl)pyrimidin-2-yl]amino]-N,N-dimethyl-benzamide;
4-[[4-[[(1S)-2-hydroxy-1-phenyl-ethyl]amino]-5-(5-methyl-1,2,4-oxadiazol-3-yl)pyrimidin-2-yl]amino]-N,2-dimethyl-benzamide;
4-[[4-[[(1S)-2-hydroxy-1-phenyl-ethyl]amino]-5-(5-methyl-1,3,4-oxadiazol-2-yl)pyrimidin-2-yl]amino]-N,N-dimethyl-benzamide;
4-[[4-[[(1S)-2-hydroxy-1-phenyl-ethyl]amino]-5-(5-methyl-1,3,4-oxadiazol-2-yl)pyrimidin-2-yl]amino]-N,2-dimethyl-benzamide;
4-[[4-[[(1S)-2-hydroxy-1-phenyl-ethyl]amino]-5-(3-methyl-1,2,4-oxadiazol-5-yl)pyrimidin-2-yl]amino]-N,N-dimethyl-benzamide;
4-[[4-[[(1S)-2-hydroxy-1-phenyl-ethyl]amino]-5-(3-methyl-1,2,4-oxadiazol-5-yl)pyrimidin-2-yl]amino]-N,2-dimethyl-benzamide;
4-[[5-(3-ethyl-1,2,4-oxadiazol-5-yl)-4-[[(1S)-2-hydroxy-1-phenyl-ethyl]amino]-pyrimidin-2-yl]amino]-2-methyl-benzamide;
4-[[5-(5-ethyl-1,3,4-oxadiazol-2-yl)-4-[[(1S)-2-hydroxy-1-phenyl-ethyl]amino]pyrimidin-2-yl]amino]-2-methyl-benzamide;
4-[[4-[[(1S)-2-hydroxy-1-phenyl-ethyl]amino]-5-(1,2,4-oxadiazol-5-yl)pyrimidin-2-yl]amino]-N,N,2-trimethyl-benzamide;
4-[[4-[[(1S)-2-hydroxy-1-phenyl-ethyl]amino]-5-(1,3,4-oxadiazol-2-yl)pyrimidin-2-yl]amino]-N,N,2-trimethyl-benzamide;
4-[[4-[[(1S)-2-hydroxy-1-phenyl-ethyl]amino]-5-(1-methyltetrazol-5-yl)pyrimidin-2-yl]amino]-N,N-dimethyl-benzamide;
4-[[4-[[(1S)-2-hydroxy-1-phenyl-ethyl]amino]-5-(1-methyltetrazol-5-yl)pyrimidin-2-yl]amino]-N,2-dimethyl-benzamide;
4-[[4-[[(1S)-2-hydroxy-1-phenyl-ethyl]amino]-5-(1H-tetrazol-5-yl)pyrimidin-2-yl]amino]-N,N,2-trimethyl-benzamide;
N-ethyl-4-[[(1S)-2-hydroxy-1-phenyl-ethyl]amino]-2-[(2-methyl-1-oxo-3,4-dihydroisoquinolin-6-yl)amino]pyrimidine-5-carboxamide;
2-(3-fluoro-4-methylsulfonyl-anilino)-4-[[(1S)-2-hydroxy-1-phenyl-ethyl]amino]pyrimidine-5-carbohydroxamic acid;
4-[[4-[[(1S)-2-hydroxy-1-phenyl-ethyl]amino]-5-(5-methyl-1,3,4-thiadiazol-2-yl)pyrimidin-2-yl]amino]-2-methyl-benzamide;
ethyl 4-[[(1S)-2-hydroxy-1-phenyl-ethyl]amino]-2-[(2-methyl-1-oxo-3,4-dihydroisoquinolin-6-yl)amino]pyrimidine-5-carboxylate;
ethyl 4-[[(1S)-2-hydroxy-1-phenyl-ethyl]amino]-2-[(2-methyl-3-oxo-1,4-dihydroisoquinolin-7-yl)amino]pyrimidine-5-carboxylate;
2-chloro-4-[[4-[[(1S)-2-hydroxy-1-phenyl-ethyl]amino]-5-(5-methyl-1H-pyrazol-3-yl)pyrimidin-2-yl]amino]benzamide;
ethyl 2-[4-(dimethylcarbamoyl)-3-methyl-anilino]-4-[[(1S)-2-hydroxy-1-phenyl-ethyl]amino]pyrimidine-5-carboxylate;
2-chloro-4-[[4-[[(1S)-2-hydroxy-1-phenyl-ethyl]amino]-5-oxazol-2-yl-pyrimidin-2-yl]amino]-N-methyl-benzamide;
2-chloro-4-[[4-[[(1S)-2-hydroxy-1-phenyl-ethyl]amino]-5-(5-methyloxazol-2-yl)pyrimidin-2-yl]amino]benzamide;
2-chloro-4-[[4-[[(1S)-2-hydroxy-1-phenyl-ethyl]amino]-5-(1H-triazol-5-yl)pyrimidin-2-yl]amino]-N-methyl-benzamide;
2-chloro-4-[[4-[[(1S)-2-hydroxy-1-phenyl-ethyl]amino]-5-(1H-1,2,4-triazol-5-yl)pyrimidin-2-yl]amino]-N-methyl-benzamide;
2-chloro-4-[[4-[[(1S)-2-hydroxy-1-phenyl-ethyl]amino]-5-(5-methyl-1H-1,2,4-triazol-3-yl)pyrimidin-2-yl]amino]benzamide;
(2S)-2-[[5-(5-methyl-1H-pyrazol-3-yl)-2-(4-methylsulfonylanilino)-pyrimidin-4-yl]amino]-2-phenyl-ethanol;
2-[3-chloro-4-(trifluoromethyl)anilino]-4-[[(1S)-2-hydroxy-1-phenyl-ethyl]amino]-N-methyl-pyrimidine-5-carboxamide;
2-chloro-4-[[4-[[(1S)-2-hydroxy-1-phenyl-ethyl]amino]-5-(1,3,4-oxadiazol-2-yl)pyrimidin-2-yl]amino]-N-methyl-benzamide;
2-chloro-4-[[4-[[(1S)-2-hydroxy-1-phenyl-ethyl]amino]-5-(1,2,4-oxadiazol-5-yl)pyrimidin-2-yl]amino]-N-methyl-benzamide;
2-chloro-4-[[4-[[(1S)-2-hydroxy-1-phenyl-ethyl]amino]-5-(3-methyl-1,2,4-oxadiazol-5-yl)pyrimidin-2-yl]amino]benzamide;
2-chloro-4-[[4-[[(1S)-2-hydroxy-1-phenyl-ethyl]amino]-5-(5-methyl-1,3,4-oxadiazol-2-yl)pyrimidin-2-yl]amino]benzamide;
2-chloro-4-[[4-[[(1S)-2-hydroxy-1-phenyl-ethyl]amino]-5-(5-methyl-1,2,4-oxadiazol-3-yl)pyrimidin-2-yl]amino]benzamide;
2-chloro-4-[[4-[[(1S)-2-hydroxy-1-phenyl-ethyl]amino]-5-(1H-tetrazol-5-yl)pyrimidin-2-yl]amino]-N-methyl-benzamide;
2-chloro-4-[[4-[[(1S)-2-hydroxy-1-phenyl-ethyl]amino]-5-(1-methyltetrazol-5-yl)pyrimidin-2-yl]amino]benzamide;
(2S)-2-[[2-(3-methyl-4-methylsulfonyl-anilino)-5-oxazol-2-yl-pyrimidin-4-yl]amino]-2-phenyl-ethanol;
(2S)-2-[[5-(5-methyloxazol-2-yl)-2-(4-methylsulfonylanilino)-pyrimidin-4-yl]amino]-2-phenyl-ethanol;
(2S)-2-[[2-(3-methyl-4-methylsulfonyl-anilino)-5-(1H-1,2,4-triazol-5-yl)pyrimidin-4-yl]amino]-2-phenyl-ethanol;
(2S)-2-[[2-(3-methyl-4-methylsulfonyl-anilino)-5-(1H-triazol-5-yl)pyrimidin-4-yl]amino]-2-phenyl-ethanol;
(2S)-2-[[2-(4-methylsulfonylanilino)-5-(5-methyl-1H-1,2,4-triazol-3-yl)pyrimidin-4-yl]amino]-2-phenyl-ethanol;
(2S)-2-[[2-(3-methyl-4-methylsulfonyl-anilino)-5-(1,3,4-oxadiazol-2-yl)pyrimidin-4-yl]amino]-2-phenyl-ethanol;
(2S)-2-[[2-(3-methyl-4-methylsulfonyl-anilino)-5-(1,2,4-oxadiazol-5-yl)pyrimidin-4-yl]amino]-2-phenyl-ethanol;
(2S)-2-[[5-(3-methyl-1,2,4-oxadiazol-5-yl)-2-(4-methylsulfonylanilino)-pyrimidin-4-yl]amino]-2-phenyl-ethanol;
(2S)-2-[[5-(5-methyl-1,3,4-oxadiazol-2-yl)-2-(4-methylsulfonylanilino)-pyrimidin-4-yl]amino]-2-phenyl-ethanol;
(2S)-2-[[5-(5-methyl-1,2,4-oxadiazol-3-yl)-2-(4-methylsulfonylanilino)-pyrimidin-4-yl]amino]-2-phenyl-ethanol;
(2S)-2-[[2-(3-methyl-4-methylsulfonyl-anilino)-5-(1H-tetrazol-5-yl)pyrimidin-4-yl]amino]-2-phenyl-ethanol;
(2S)-2-[[2-(4-methylsulfonylanilino)-5-(1-methyltetrazol-5-yl)pyrimidin-4-yl]amino]-2-phenyl-ethanol;
ethyl 2-[3-fluoro-4-(2-hydroxy-1,1-dimethyl-ethyl)anilino]-4-[[(1S)-2-hydroxy-1-phenyl-ethyl]amino]pyrimidine-5-carboxylate;
(2S)-2-[[2-(3-fluoro-4-methylsulfonyl-anilino)-5-oxazol-2-yl-pyrimidin-4-yl]amino]-2-phenyl-ethanol;
(2S)-2-[[2-(3-fluoro-4-methylsulfonyl-anilino)-5-(1H-1,2,4-triazol-5-yl)pyrimidin-4-yl]amino]-2-phenyl-ethanol;
(2S)-2-[[2-(3-fluoro-4-methylsulfonyl-anilino)-5-(1H-triazol-5-yl)pyrimidin-4-yl]amino]-2-phenyl-ethanol;
N-ethyl-4-[[(1S)-2-hydroxy-1-phenyl-ethyl]amino]-2-(3-methyl-4-methylsulfonyl-anilino)pyrimidine-5-carboxamide;
4-[[(1S)-2-hydroxy-1-phenyl-ethyl]amino]-N,N-dimethyl-2-(3-methyl-4-methylsulfonyl-anilino)pyrimidine-5-carboxamide;
2-[3-chloro-4-(2-hydroxy-1,1-dimethyl-ethyl)anilino]-4-[[(1S)-2-hydroxy-1-phenyl-ethyl]amino]-N-methyl-pyrimidine-5-carboxamide;
6-[[4-[[(1S)-2-hydroxy-1-phenyl-ethyl]amino]-5-(5-methyl-1H-pyrazol-3-yl)pyrimidin-2-yl]amino]-2-methyl-3,4-dihydroisoquinolin-1-one;
7-[[4-[[(1S)-2-hydroxy-1-phenyl-ethyl]amino]-5-(5-methyl-1H-pyrazol-3-yl)pyrimidin-2-yl]amino]-2-methyl-1,4-dihydroisoquinolin-3-one;
(2S)-2-[[2-(3-fluoro-4-methylsulfonyl-anilino)-5-(1,3,4-oxadiazol-2-yl)pyrimidin-4-yl]amino]-2-phenyl-ethanol;
(2S)-2-[[2-(3-fluoro-4-methylsulfonyl-anilino)-5-(1,2,4-oxadiazol-5-yl)pyrimidin-4-yl]amino]-2-phenyl-ethanol;
ethyl 2-[(3,3-dioxo-1,3lambda6-benzoxathiol-6-yl)amino]-4-[[(1S)-2-hydroxy-1-phenyl-ethyl]amino]pyrimidine-5-carboxylate;
ethyl 2-[(3,3-dioxo-1,3lambda6-benzoxathiol-5-yl)amino]-4-[[(1S)-2-hydroxy-1-phenyl-ethyl]amino]pyrimidine-5-carboxylate;
(2S)-2-[[2-(3-fluoro-4-methylsulfonyl-anilino)-5-(1H-tetrazol-5-yl)pyrimidin-4-yl]amino]-2-phenyl-ethanol;
ethyl 4-[[(1S)-2-hydroxy-1-phenyl-ethyl]amino]-2-(3-methyl-4-methylsulfonyl-anilino)pyrimidine-5-carboxylate;
ethyl 4-[[(1S)-2-hydroxy-1-phenyl-ethyl]amino]-2-(2-methyl-4-methylsulfonyl-anilino)pyrimidine-5-carboxylate;
ethyl 2-(4-ethylsulfonylanilino)-4-[[(1S)-2-hydroxy-1-phenyl-ethyl]amino]pyrimidine-5-carboxylate;
ethyl 4-[[(1R)-2-hydroxy-1-phenyl-ethyl]amino]-2-(3-methyl-4-methylsulfonyl-anilino)pyrimidine-5-carboxylate;
7-[[4-[[(1S)-2-hydroxy-1-phenyl-ethyl]amino]-5-oxazol-2-yl-pyrimidin-2-yl]amino]-3,3-dimethyl-2,4-dihydroisoquinolin-1-one;
6-[[4-[[(1S)-2-hydroxy-1-phenyl-ethyl]amino]-5-(5-methyloxazol-2-yl)pyrimidin-2-yl]amino]-2-methyl-3,4-dihydroisoquinolin-1-one;
7-[[4-[[(1S)-2-hydroxy-1-phenyl-ethyl]amino]-5-(5-methyloxazol-2-yl)pyrimidin-2-yl]amino]-2-methyl-1,4-dihydroisoquinolin-3-one;
7-[[4-[[(1S)-2-hydroxy-1-phenyl-ethyl]amino]-5-(1H-triazol-5-yl)pyrimidin-2-yl]amino]-3,3-dimethyl-2,4-dihydroisoquinolin-1-one;
7-[[4-[[(1S)-2-hydroxy-1-phenyl-ethyl]amino]-5-(1H-1,2,4-triazol-5-yl)pyrimidin-2-yl]amino]-3,3-dimethyl-2,4-dihydroisoquinolin-1-one;
6-[[4-[[(1S)-2-hydroxy-1-phenyl-ethyl]amino]-5-(5-methyl-1H-1,2,4-triazol-3-yl)pyrimidin-2-yl]amino]-2-methyl-3,4-dihydroisoquinolin-1-one;
7-[[4-[[(1S)-2-hydroxy-1-phenyl-ethyl]amino]-5-(5-methyl-1H-1,2,4-triazol-3-yl)pyrimidin-2-yl]amino]-2-methyl-1,4-dihydroisoquinolin-3-one;
6-[[5-(5-ethyl-1,3,4-oxadiazol-2-yl)-4-[[(1S)-2-hydroxy-1-phenyl-ethyl]amino]pyrimidin-2-yl]amino]-3,4-dihydro-2H-isoquinolin-1-one;
6-[[5-(5-ethyl-1,3,4-oxadiazol-2-yl)-4-[[(1S)-2-hydroxy-1-phenyl-ethyl]amino]pyrimidin-2-yl]amino]-3,4-dihydro-1H-quinolin-2-one;
6-[[5-(3-ethyl-1,2,4-oxadiazol-5-yl)-4-[[(1S)-2-hydroxy-1-phenyl-ethyl]amino]pyrimidin-2-yl]amino]-3,4-dihydro-2H-isoquinolin-1-one;
6-[[5-(3-ethyl-1,2,4-oxadiazol-5-yl)-4-[[(1S)-2-hydroxy-1-phenyl-ethyl]amino]pyrimidin-2-yl]amino]-3,4-dihydro-1H-quinolin-2-one;
7-[[4-[[(1S)-2-hydroxy-1-phenyl-ethyl]amino]-5-(1,3,4-oxadiazol-2-yl)pyrimidin-2-yl]amino]-3,3-dimethyl-2,4-dihydroisoquinolin-1-one;
7-[[4-[[(1S)-2-hydroxy-1-phenyl-ethyl]amino]-5-(1,2,4-oxadiazol-5-yl)pyrimidin-2-yl]amino]-3,3-dimethyl-2,4-dihydroisoquinolin-1-one;
6-[[4-[[(1S)-2-hydroxy-1-phenyl-ethyl]amino]-5-(3-methyl-1,2,4-oxadiazol-5-yl)pyrimidin-2-yl]amino]-2-methyl-3,4-dihydroisoquinolin-1-one;
7-[[4-[[(1S)-2-hydroxy-1-phenyl-ethyl]amino]-5-(3-methyl-1,2,4-oxadiazol-5-yl)pyrimidin-2-yl]amino]-2-methyl-1,4-dihydroisoquinolin-3-one;
6-[[4-[[(1S)-2-hydroxy-1-phenyl-ethyl]amino]-5-(5-methyl-1,3,4-oxadiazol-2-yl)pyrimidin-2-yl]amino]-2-methyl-3,4-dihydroisoquinolin-1-one;
7-[[4-[[(1S)-2-hydroxy-1-phenyl-ethyl]amino]-5-(5-methyl-1,3,4-oxadiazol-2-yl)pyrimidin-2-yl]amino]-2-methyl-1,4-dihydroisoquinolin-3-one;
6-[[4-[[(1S)-2-hydroxy-1-phenyl-ethyl]amino]-5-(5-methyl-1,2,4-oxadiazol-3-yl)pyrimidin-2-yl]amino]-2-methyl-3,4-dihydroisoquinolin-1-one;
7-[[4-[[(1S)-2-hydroxy-1-phenyl-ethyl]amino]-5-(5-methyl-1,2,4-oxadiazol-3-yl)pyrimidin-2-yl]amino]-2-methyl-1,4-dihydroisoquinolin-3-one;
N-ethyl-2-(4-fluoro-3-phenyl-anilino)-4-[[(1S)-2-hydroxy-1-phenyl-ethyl]amino]pyrimidine-5-carboxamide;
7-[[4-[[(1S)-2-hydroxy-1-phenyl-ethyl]amino]-5-(1H-tetrazol-5-yl)pyrimidin-2-yl]amino]-3,3-dimethyl-2,4-dihydroisoquinolin-1-one;
6-[[4-[[(1S)-2-hydroxy-1-phenyl-ethyl]amino]-5-(1-methyltetrazol-5-yl)pyrimidin-2-yl]amino]-2-methyl-3,4-dihydroisoquinolin-1-one;
7-[[4-[[(1S)-2-hydroxy-1-phenyl-ethyl]amino]-5-(1-methyltetrazol-5-yl)pyrimidin-2-yl]amino]-2-methyl-1,4-dihydroisoquinolin-3-one;
4-[[4-[[(1S)-2-hydroxy-1-phenyl-ethyl]amino]-5-(5-methyl-1H-pyrazol-3-yl)pyrimidin-2-yl]amino]-N,N,2-trimethyl-benzamide;
ethyl 4-[(2-fluoro-1-phenyl-ethyl)amino]-2-(3-methyl-4-methylsulfonyl-anilino)pyrimidine-5-carboxylate;
ethyl 2-(4-fluoro-3-phenyl-anilino)-4-[[(1S)-2-hydroxy-1-phenyl-ethyl]amino]pyrimidine-5-carboxylate;
4-[[4-[[(1S)-2-hydroxy-1-phenyl-ethyl]amino]-5-(5-methyloxazol-2-yl)pyrimidin-2-yl]amino]-N,N,2-trimethyl-benzamide;
4-[[4-[[(1S)-2-hydroxy-1-phenyl-ethyl]amino]-5-(5-methyl-1H-1,2,4-triazol-3-yl)pyrimidin-2-yl]amino]-N,N,2-trimethyl-benzamide;
6-[[4-[[(1S)-2-hydroxy-1-phenyl-ethyl]amino]-5-(5-methyl-1,3,4-thiadiazol-2-yl)pyrimidin-2-yl]amino]-3,4-dihydro-2H-isoquinolin-1-one;
6-[[4-[[(1S)-2-hydroxy-1-phenyl-ethyl]amino]-5-(5-methyl-1,3,4-thiadiazol-2-yl)pyrimidin-2-yl]amino]-3,4-dihydro-1H-quinolin-2-one;
4-[[5-(5-ethyl-1,3,4-oxadiazol-2-yl)-4-[[(1S)-2-hydroxy-1-phenyl-ethyl]amino]pyrimidin-2-yl]amino]-N,N-dimethyl-benzamide;
4-[[5-(5-ethyl-1,3,4-oxadiazol-2-yl)-4-[[(1S)-2-hydroxy-1-phenyl-ethyl]amino]pyrimidin-2-yl]amino]-N,2-dimethyl-benzamide;
4-[[5-(3-ethyl-1,2,4-oxadiazol-5-yl)-4-[[(1S)-2-hydroxy-1-phenyl-ethyl]amino]pyrimidin-2-yl]amino]-N,N-dimethyl-benzamide;
4-[[5-(3-ethyl-1,2,4-oxadiazol-5-yl)-4-[[(1S)-2-hydroxy-1-phenyl-ethyl]amino]pyrimidin-2-yl]amino]-N,2-dimethyl-benzamide;
4-[[4-[[(1S)-2-hydroxy-1-phenyl-ethyl]amino]-5-(3-isopropyl-1,2,4-oxadiazol-5-yl)pyrimidin-2-yl]amino]-2-methyl-benzamide;
4-[[4-[[(1S)-2-hydroxy-1-phenyl-ethyl]amino]-5-(5-isopropyl-1,3,4-oxadiazol-2-yl)pyrimidin-2-yl]amino]-2-methyl-benzamide;
4-[[4-[[(1S)-2-hydroxy-1-phenyl-ethyl]amino]-5-(3-methyl-1,2,4-oxadiazol-5-yl)pyrimidin-2-yl]amino]-N,N,2-trimethyl-benzamide;
4-[[4-[[(1S)-2-hydroxy-1-phenyl-ethyl]amino]-5-(5-methyl-1,3,4-oxadiazol-2-yl)pyrimidin-2-yl]amino]-N,N,2-trimethyl-benzamide;
4-[[4-[[(1S)-2-hydroxy-1-phenyl-ethyl]amino]-5-(5-methyl-1,2,4-oxadiazol-3-yl)pyrimidin-2-yl]amino]-N,N,2-trimethyl-benzamide;
4-[[4-[[(1S)-2-hydroxy-1-phenyl-ethyl]amino]-5-(1-methyltetrazol-5-yl)pyrimidin-2-yl]amino]-N,N,2-trimethyl-benzamide;
ethyl 2-(3-fluoro-4-methylsulfonyl-anilino)-4-[[(1S)-2-hydroxy-1-phenyl-ethyl]amino]-pyrimidine-5-carboxylate;
ethyl 2-(2-fluoro-4-methylsulfonyl-anilino)-4-[[(1S)-2-hydroxy-1-phenyl-ethyl]amino]pyrimidine-5-carboxylate;
4-[[(1S)-2-hydroxy-1-phenyl-ethyl]amino]-N-isopropyl-2-[(2-methyl-1-oxo-3,4-dihydroisoquinolin-6-yl)amino]pyrimidine-5-carboxamide;
(2S)-2-[[2-[(1,1-dioxo-2H-thiochromen-6-yl)amino]-5-oxazol-2-yl-pyrimidin-4-yl]amino]-2-phenyl-ethanol;
(2S)-2-[[2-[(1,1-dioxo-2H-thiochromen-6-yl)amino]-5-(1H-triazol-5-yl)pyrimidin-4-yl]amino]-2-phenyl-ethanol;
(2S)-2-[[2-[(1,1-dioxo-2H-thiochromen-6-yl)amino]-5-(1H-1,2,4-triazol-5-yl)pyrimidin-4-yl]amino]-2-phenyl-ethanol;
4-[[4-[[(1S)-2-hydroxy-1-phenyl-ethyl]amino]-5-(5-methyl-1,3,4-thiadiazol-2-yl)pyrimidin-2-yl]amino]-N,N-dimethyl-benzamide;
4-[[4-[[(1S)-2-hydroxy-1-phenyl-ethyl]amino]-5-(5-methyl-1,3,4-thiadiazol-2-yl)pyrimidin-2-yl]amino]-N,2-dimethyl-benzamide;
ethyl 2-[(2-ethyl-1-oxo-3,4-dihydroisoquinolin-6-yl)amino]-4-[[(1S)-2-hydroxy-1-phenyl-ethyl]amino]pyrimidine-5-carboxylate;
(2S)-2-[[2-[(1,1-dioxo-2H-thiochromen-6-yl)amino]-5-(1,2,4-oxadiazol-5-yl)pyrimidin-4-yl]amino]-2-phenyl-ethanol;
(2S)-2-[[2-[(1,1-dioxo-2H-thiochromen-6-yl)amino]-5-(1,3,4-oxadiazol-2-yl)pyrimidin-4-yl]amino]-2-phenyl-ethanol;
(2S)-2-[[2-[(1,1-dioxo-2H-thiochromen-6-yl)amino]-5-(1H-tetrazol-5-yl)pyrimidin-4-yl]amino]-2-phenyl-ethanol;
ethyl 4-[[(1S)-1-cyclohexyl-2-hydroxy-ethyl]amino]-2-(3-methyl-4-methylsulfonyl-anilino)pyrimidine-5-carboxylate;
(2S)-2-[[2-[(1,1-dioxo-3,4-dihydro-2H-thiochromen-6-yl)amino]-5-oxazol-2-yl-pyrimidin-4-yl]amino]-2-phenyl-ethanol;
(2S)-2-[[2-[(1,1-dioxo-3,4-dihydro-2H-thiochromen-6-yl)amino]-5-(1H-triazol-5-yl)pyrimidin-4-yl]amino]-2-phenyl-ethanol;
(2S)-2-[[2-[(1,1-dioxo-3,4-dihydro-2H-thiochromen-6-yl)amino]-5-(1H-1,2,4-triazol-5-yl)pyrimidin-4-yl]amino]-2-phenyl-ethanol;
2-chloro-4-[[4-[[(1S)-2-hydroxy-1-phenyl-ethyl]amino]-5-(5-methyl-1H-pyrazol-3-yl)pyrimidin-2-yl]amino]-N-methyl-benzamide;
(2S)-2-[[2-[(1,1-dioxo-3,4-dihydro-2H-thiochromen-6-yl)amino]-5-(1,2,4-oxadiazol-5-yl)pyrimidin-4-yl]amino]-2-phenyl-ethanol;
(2S)-2-[[2-[(1,1-dioxo-3,4-dihydro-2H-thiochromen-6-yl)amino]-5-(1,3,4-oxadiazol-2-yl)pyrimidin-4-yl]amino]-2-phenyl-ethanol;
2-chloro-4-[[4-[[(1S)-2-hydroxy-1-phenyl-ethyl]amino]-5-(5-methyloxazol-2-yl)pyrimidin-2-yl]amino]-N-methyl-benzamide;
2-chloro-4-[[4-[[(1S)-2-hydroxy-1-phenyl-ethyl]amino]-5-oxazol-2-yl-pyrimidin-2-yl]amino]-N,N-dimethyl-benzamide;
(2S)-2-[[2-[(1,1-dioxo-3,4-dihydro-2H-thiochromen-6-yl)amino]-5-(1H-tetrazol-5-yl)pyrimidin-4-yl]amino]-2-phenyl-ethanol;
2-chloro-4-[[4-[[(1S)-2-hydroxy-1-phenyl-ethyl]amino]-5-(5-methyl-1H-1,2,4-triazol-3-yl)pyrimidin-2-yl]amino]-N-methyl-benzamide;
2-chloro-4-[[4-[[(1S)-2-hydroxy-1-phenyl-ethyl]amino]-5-(1H-triazol-5-yl)pyrimidin-2-yl]amino]-N,N-dimethyl-benzamide;
2-chloro-4-[[4-[[(1S)-2-hydroxy-1-phenyl-ethyl]amino]-5-(1H-1,2,4-triazol-5-yl)pyrimidin-2-yl]amino]-N,N-dimethyl-benzamide;
(2S)-2-[[2-(3-methyl-4-methylsulfonyl-anilino)-5-(5-methyl-1H-pyrazol-3-yl)pyrimidin-4-yl]amino]-2-phenyl-ethanol;
2-chloro-4-[[4-[[(1S)-2-hydroxy-1-phenyl-ethyl]amino]-5-(5-methyl-1,2,4-oxadiazol-3-yl)pyrimidin-2-yl]amino]-N-methyl-benzamide;
2-chloro-4-[[4-[[(1S)-2-hydroxy-1-phenyl-ethyl]amino]-5-(5-methyl-1,3,4-oxadiazol-2-yl)pyrimidin-2-yl]amino]-N-methyl-benzamide;
2-chloro-4-[[4-[[(1S)-2-hydroxy-1-phenyl-ethyl]amino]-5-(3-methyl-1,2,4-oxadiazol-5-yl)pyrimidin-2-yl]amino]-N-methyl-benzamide;
2-chloro-4-[[4-[[(1S)-2-hydroxy-1-phenyl-ethyl]amino]-5-(1,2,4-oxadiazol-5-yl)pyrimidin-2-yl]amino]-N,N-dimethyl-benzamide;
2-chloro-4-[[4-[[(1S)-2-hydroxy-1-phenyl-ethyl]amino]-5-(1,3,4-oxadiazol-2-yl)pyrimidin-2-yl]amino]-N,N-dimethyl-benzamide;
2-chloro-4-[[5-(3-ethyl-1,2,4-oxadiazol-5-yl)-4-[[(1S)-2-hydroxy-1-phenyl-ethyl]amino]pyrimidin-2-yl]amino]benzamide;
2-chloro-4-[[5-(5-ethyl-1,3,4-oxadiazol-2-yl)-4-[[(1S)-2-hydroxy-1-phenyl-ethyl]amino]pyrimidin-2-yl]amino]benzamide;
2-chloro-4-[[4-[[(1S)-2-hydroxy-1-phenyl-ethyl]amino]-5-(1-methyltetrazol-5-yl)pyrimidin-2-yl]amino]-N-methyl-benzamide;
2-chloro-4-[[4-[[(1S)-2-hydroxy-1-phenyl-ethyl]amino]-5-(1H-tetrazol-5-yl)pyrimidin-2-yl]amino]-N,N-dimethyl-benzamide;
(2S)-2-[[2-(3-methyl-4-methylsulfonyl-anilino)-5-(5-methyloxazol-2-yl)pyrimidin-4-yl]amino]-2-phenyl-ethanol;
(2S)-2-[[2-(3-methyl-4-methylsulfonyl-anilino)-5-(5-methyl-1H-1,2,4-triazol-3-yl)pyrimidin-4-yl]amino]-2-phenyl-ethanol;
ethyl 2-[4-(dimethylcarbamoyl)-3-methoxy-anilino]-4-[[(1S)-2-hydroxy-1-phenyl-ethyl]amino]pyrimidine-5-carboxylate;
ethyl 2-[3-chloro-4-(trifluoromethyl)anilino]-4-[[(1S)-2-hydroxy-1-phenyl-ethyl]amino]pyrimidine-5-carboxylate;
(2S)-2-[[5-(3-ethyl-1,2,4-oxadiazol-5-yl)-2-(4-methylsulfonylanilino)-pyrimidin-4-yl]amino]-2-phenyl-ethanol;
(2S)-2-[[5-(5-ethyl-1,3,4-oxadiazol-2-yl)-2-(4-methylsulfonyl-anilino)pyrimidin-4-yl]amino]-2-phenyl-ethanol;
(2S)-2-[[2-(3-methyl-4-methylsulfonyl-anilino)-5-(5-methyl-1,3,4-oxadiazol-2-yl)pyrimidin-4-yl]amino]-2-phenyl-ethanol;
(2S)-2-[[2-(3-methyl-4-methylsulfonyl-anilino)-5-(3-methyl-1,2,4-oxadiazol-5-yl)pyrimidin-4-yl]amino]-2-phenyl-ethanol;
(2S)-2-[[2-(3-methyl-4-methylsulfonyl-anilino)-5-(5-methyl-1,2,4-oxadiazol-3-yl)pyrimidin-4-yl]amino]-2-phenyl-ethanol;
(2S)-2-[[2-(3-methyl-4-methylsulfonyl-anilino)-5-(1-methyltetrazol-5-yl)pyrimidin-4-yl]amino]-2-phenyl-ethanol;
2-chloro-4-[[4-[[(1S)-2-hydroxy-1-phenyl-ethyl]amino]-5-(5-methyl-1,3,4-thiadiazol-2-yl)pyrimidin-2-yl]amino]benzamide;
4-[[5-[5-(difluoromethyl)-1,3,4-oxadiazol-2-yl]-4-[[(1S)-2-hydroxy-1-phenyl-ethyl]amino]pyrimidin-2-yl]amino]-2-methyl-benzamide;
4-[[5-[3-(difluoromethyl)-1,2,4-oxadiazol-5-yl]-4-[[(1S)-2-hydroxy-1-phenyl-ethyl]amino]pyrimidin-2-yl]amino]-2-methyl-benzamide;
(2S)-2-[[2-(4-methylsulfonylanilino)-5-(5-methyl-1,3,4-thiadiazol-2-yl)pyrimidin-4-yl]amino]-2-phenyl-ethanol;
(2S)-2-[[2-(3-fluoro-4-methylsulfonyl-anilino)-5-(5-methyl-1H-pyrazol-3-yl)pyrimidin-4-yl]amino]-2-phenyl-ethanol;
ethyl 4-[[(1S,2R)-2-hydroxyindan-1-yl]amino]-2-(3-methyl-4-methylsulfonyl-anilino)pyrimidine-5-carboxylate;
ethyl 4-[[(1S,2S)-2-hydroxyindan-1-yl]amino]-2-(3-methyl-4-methylsulfonyl-anilino)pyrimidine-5-carboxylate;
ethyl 4-[[(1R,2S)-2-hydroxyindan-1-yl]amino]-2-(3-methyl-4-methylsulfonyl-anilino)pyrimidine-5-carboxylate;
ethyl 4-[(2S)-2-(hydroxymethyl)-indolin-1-yl]-2-(3-methyl-4-methylsulfonyl-anilino)pyrimidine-5-carboxylate;
(2S)-2-[[2-(3-fluoro-4-methylsulfonyl-anilino)-5-(5-methyloxazol-2-yl)pyrimidin-4-yl]amino]-2-phenyl-ethanol;
(2S)-2-[[2-(3-fluoro-4-methylsulfonyl-anilino)-5-(5-methyl-1H-1,2,4-triazol-3-yl)pyrimidin-4-yl]amino]-2-phenyl-ethanol;
ethyl 2-[3-chloro-4-(dimethylcarbamoyl)anilino]-4-[[(1S)-2-hydroxy-1-phenyl-ethyl]amino]pyrimidine-5-carboxylate;
4-[[(1S)-2-hydroxy-1-phenyl-ethyl]amino]-N-isopropyl-2-(3-methyl-4-methylsulfonyl-anilino)pyrimidine-5-carboxamide;
7-[[4-[[(1S)-2-hydroxy-1-phenyl-ethyl]amino]-5-(5-methyl-1H-pyrazol-3-yl)pyrimidin-2-yl]amino]-3,3-dimethyl-2,4-dihydroisoquinolin-1-one;
(2S)-2-[[2-(3-fluoro-4-methylsulfonyl-anilino)-5-(5-methyl-1,3,4-oxadiazol-2-yl)pyrimidin-4-yl]amino]-2-phenyl-ethanol;
(2S)-2-[[2-(3-fluoro-4-methylsulfonyl-anilino)-5-(3-methyl-1,2,4-oxadiazol-5-yl)pyrimidin-4-yl]amino]-2-phenyl-ethanol;
(2S)-2-[[2-(3-fluoro-4-methylsulfonyl-anilino)-5-(5-methyl-1,2,4-oxadiazol-3-yl)pyrimidin-4-yl]amino]-2-phenyl-ethanol;
(2S)-2-[[2-(3-fluoro-4-methylsulfonyl-anilino)-5-(1-methyltetrazol-5-yl)pyrimidin-4-yl]amino]-2-phenyl-ethanol;
ethyl 4-[[(1S)-1-benzyl-2-hydroxy-ethyl]amino]-2-(3-methyl-4-methylsulfonyl-anilino)pyrimidine-5-carboxylate;
ethyl 4-[[(1S)-2-hydroxy-1-phenyl-ethyl]amino]-2-(4-isopropylsulfonylanilino)pyrimidine-5-carboxylate;
ethyl 2-[3-chloro-4-(2-hydroxy-1,1-dimethyl-ethyl)anilino]-4-[[(1S)-2-hydroxy-1-phenyl-ethyl]amino]pyrimidine-5-carboxylate;
7-[[4-[[(1S)-2-hydroxy-1-phenyl-ethyl]amino]-5-(5-methyloxazol-2-yl)pyrimidin-2-yl]amino]-3,3-dimethyl-2,4-dihydroisoquinolin-1-one;
7-[[4-[[(1S)-2-hydroxy-1-phenyl-ethyl]amino]-5-(5-methyl-1H-1,2,4-triazol-3-yl)pyrimidin-2-yl]amino]-3,3-dimethyl-2,4-dihydroisoquinolin-1-one;
(2S)-2-[[2-(3-chloro-4-methylsulfonyl-anilino)-5-oxazol-2-yl-pyrimidin-4-yl]amino]-2-phenyl-ethanol;
(2S)-2-[[2-(3-chloro-4-methylsulfonyl-anilino)-5-(1H-triazol-5-yl)pyrimidin-4-yl]amino]-2-phenyl-ethanol;
(2S)-2-[[2-(3-chloro-4-methylsulfonyl-anilino)-5-(1H-1,2,4-triazol-5-yl)pyrimidin-4-yl]amino]-2-phenyl-ethanol;
ethyl 4-[[(1S)-1-(hydroxymethyl)-2-(3-pyridyl)ethyl]amino]-2-(3-methyl-4-methylsulfonyl-anilino)pyrimidine-5-carboxylate;
6-[[4-[[(1S)-2-hydroxy-1-phenyl-ethyl]amino]-5-(5-isopropyl-1,3,4-oxadiazol-2-yl)pyrimidin-2-yl]amino]-3,4-dihydro-2H-isoquinolin-1-one;
6-[[4-[[(1S)-2-hydroxy-1-phenyl-ethyl]amino]-5-(5-isopropyl-1,3,4-oxadiazol-2-yl)pyrimidin-2-yl]amino]-3,4-dihydro-1H-quinolin-2-one;
6-[[4-[[(1S)-2-hydroxy-1-phenyl-ethyl]amino]-5-(3-isopropyl-1,2,4-oxadiazol-5-yl)pyrimidin-2-yl]amino]-3,4-dihydro-2H-isoquinolin-1-one;
6-[[4-[[(1S)-2-hydroxy-1-phenyl-ethyl]amino]-5-(3-isopropyl-1,2,4-oxadiazol-5-yl)pyrimidin-2-yl]amino]-3,4-dihydro-1H-quinolin-2-one;
7-[[4-[[(1S)-2-hydroxy-1-phenyl-ethyl]amino]-5-(5-methyl-1,2,4-oxadiazol-3-yl)pyrimidin-2-yl]amino]-3,3-dimethyl-2,4-dihydroisoquinolin-1-one;
7-[[4-[[(1S)-2-hydroxy-1-phenyl-ethyl]amino]-5-(5-methyl-1,3,4-oxadiazol-2-yl)pyrimidin-2-yl]amino]-3,3-dimethyl-2,4-dihydroisoquinolin-1-one;
7-[[4-[[(1S)-2-hydroxy-1-phenyl-ethyl]amino]-5-(3-methyl-1,2,4-oxadiazol-5-yl)pyrimidin-2-yl]amino]-3,3-dimethyl-2,4-dihydroisoquinolin-1-one;
6-[[5-(3-ethyl-1,2,4-oxadiazol-5-yl)-4-[[(1S)-2-hydroxy-1-phenyl-ethyl]amino]pyrimidin-2-yl]amino]-2-methyl-3,4-dihydroisoquinolin-1-one;
7-[[5-(3-ethyl-1,2,4-oxadiazol-5-yl)-4-[[(1S)-2-hydroxy-1-phenyl-ethyl]amino]pyrimidin-2-yl]amino]-2-methyl-1,4-dihydroisoquinolin-3-one;
6-[[5-(5-ethyl-1,3,4-oxadiazol-2-yl)-4-[[(1S)-2-hydroxy-1-phenyl-ethyl]amino]pyrimidin-2-yl]amino]-2-methyl-3,4-dihydroisoquinolin-1-one;
7-[[5-(5-ethyl-1,3,4-oxadiazol-2-yl)-4-[[(1S)-2-hydroxy-1-phenyl-ethyl]amino]pyrimidin-2-yl]amino]-2-methyl-1,4-dihydroisoquinolin-3-one;
2-(4-fluoro-3-phenyl-anilino)-4-[[(1S)-2-hydroxy-1-phenyl-ethyl]amino]-N-isopropyl-pyrimidine-5-carboxamide;
7-[[4-[[(1S)-2-hydroxy-1-phenyl-ethyl]amino]-5-(1-methyltetrazol-5-yl)pyrimidin-2-yl]amino]-3,3-dimethyl-2,4-dihydroisoquinolin-1-one;
(2S)-2-[[2-(3-chloro-4-methylsulfonyl-anilino)-5-(1,3,4-oxadiazol-2-yl)pyrimidin-4-yl]amino]-2-phenyl-ethanol;
(2S)-2-[[2-(3-chloro-4-methylsulfonyl-anilino)-5-(1,2,4-oxadiazol-5-yl)pyrimidin-4-yl]amino]-2-phenyl-ethanol;
(2S)-2-[[2-(3-chloro-4-methylsulfonyl-anilino)-5-(1H-tetrazol-5-yl)pyrimidin-4-yl]amino]-2-phenyl-ethanol;
ethyl 2-(3-fluoro-4-methylsulfonyl-anilino)-4-[[(1S,2R)-2-hydroxyindan-1-yl]amino]pyrimidine-5-carboxylate;
ethyl 4-[[(1S)-2-hydroxy-1-phenyl-ethyl]amino]-2-(2-methoxy-4-methylsulfonyl-anilino)pyrimidine-5-carboxylate;
ethyl 4-[[(1S)-2-hydroxy-1-phenyl-ethyl]amino]-2-(3-methoxy-4-methylsulfonyl-anilino)pyrimidine-5-carboxylate;
2-(3-fluoro-4-methylsulfonyl-anilino)-4-[[(1S)-2-hydroxy-1-phenyl-ethyl]amino]-N-isopropyl-pyrimidine-5-carboxamide;
7-[[4-[[(1S)-2-hydroxy-1-phenyl-ethyl]amino]-5-(5-methyl-1,3,4-thiadiazol-2-yl)pyrimidin-2-yl]amino]-2-methyl-1,4-dihydroisoquinolin-3-one;
6-[[4-[[(1S)-2-hydroxy-1-phenyl-ethyl]amino]-5-(5-methyl-1,3,4-thiadiazol-2-yl)pyrimidin-2-yl]amino]-2-methyl-3,4-dihydroisoquinolin-1-one;
4-[[4-[[(1S)-2-hydroxy-1-phenyl-ethyl]amino]-5-(5-isopropyl-1,3,4-oxadiazol-2-yl)pyrimidin-2-yl]amino]-N,N-dimethyl-benzamide;
4-[[4-[[(1S)-2-hydroxy-1-phenyl-ethyl]amino]-5-(5-isopropyl-1,3,4-oxadiazol-2-yl)pyrimidin-2-yl]amino]-N,2-dimethyl-benzamide;
4-[[4-[[(1S)-2-hydroxy-1-phenyl-ethyl]amino]-5-(3-isopropyl-1,2,4-oxadiazol-5-yl)pyrimidin-2-yl]amino]-N,N-dimethyl-benzamide;
4-[[4-[[(1S)-2-hydroxy-1-phenyl-ethyl]amino]-5-(3-isopropyl-1,2,4-oxadiazol-5-yl)pyrimidin-2-yl]amino]-N,2-dimethyl-benzamide;
4-[[5-(3-tert-butyl-1,2,4-oxadiazol-5-yl)-4-[[(1S)-2-hydroxy-1-phenyl-ethyl]amino]pyrimidin-2-yl]amino]-2-methyl-benzamide;
4-[[5-(5-tert-butyl-1,3,4-oxadiazol-2-yl)-4-[[(1S)-2-hydroxy-1-phenyl-ethyl]amino]pyrimidin-2-yl]amino]-2-methyl-benzamide;
4-[[5-(3-ethyl-1,2,4-oxadiazol-5-yl)-4-[[(1S)-2-hydroxy-1-phenyl-ethyl]amino]pyrimidin-2-yl]amino]-N,N,2-trimethyl-benzamide;
4-[[5-(5-ethyl-1,3,4-oxadiazol-2-yl)-4-[[(1S)-2-hydroxy-1-phenyl-ethyl]amino]pyrimidin-2-yl]amino]-N,N,2-trimethyl-benzamide;
(2S)-2-[[2-[(1,1-dioxo-2H-thiochromen-6-yl)amino]-5-(5-methyl-1H-pyrazol-3-yl)pyrimidin-4-yl]amino]-2-phenyl-ethanol;
(2S)-2-[[2-[(1,1-dioxo-2H-thiochromen-6-yl)amino]-5-(5-methyloxazol-2-yl)pyrimidin-4-yl]amino]-2-phenyl-ethanol;
(2S)-2-[[2-[(1,1-dioxo-2H-thiochromen-6-yl)amino]-5-(5-methyl-1H-1,2,4-triazol-3-yl)pyrimidin-4-yl]amino]-2-phenyl-ethanol;
4-[[4-[[(1S)-2-hydroxy-1-phenyl-ethyl]amino]-5-(5-methyl-1,3,4-thiadiazol-2-yl)pyrimidin-2-yl]amino]-N,N,2-trimethyl-benzamide;
ethyl 4-[[(1S)-2-hydroxy-1-phenyl-ethyl]amino]-2-[(2-isopropyl-1-oxo-3,4-dihydroisoquinolin-6-yl)amino]pyrimidine-5-carboxylate;
ethyl 2-(2-chloro-4-methylsulfonyl-anilino)-4-[[(1S)-2-hydroxy-1-phenyl-ethyl]amino]pyrimidine-5-carboxylate;
ethyl 2-(3-chloro-4-methylsulfonyl-anilino)-4-[[(1S)-2-hydroxy-1-phenyl-ethyl]amino]-pyrimidine-5-carboxylate;
2-hydroxyethyl 2-(3-fluoro-4-methylsulfonyl-anilino)-4-[[(1S)-2-hydroxy-1-phenyl-ethyl]amino]pyrimidine-5-carboxylate;
(2S)-2-[[2-[(1,1-dioxo-2H-thiochromen-6-yl)amino]-5-(3-methyl-1,2,4-oxadiazol-5-yl)pyrimidin-4-yl]amino]-2-phenyl-ethanol;
(2S)-2-[[2-[(1,1-dioxo-2H-thiochromen-6-yl)amino]-5-(5-methyl-1,3,4-oxadiazol-2-yl)pyrimidin-4-yl]amino]-2-phenyl-ethanol;
(2S)-2-[[2-[(1,1-dioxo-2H-thiochromen-6-yl)amino]-5-(5-methyl-1,2,4-oxadiazol-3-yl)pyrimidin-4-yl]amino]-2-phenyl-ethanol;
(2S)-2-[[2-[(1,1-dioxo-2H-thiochromen-6-yl)amino]-5-(1-methyltetrazol-5-yl)pyrimidin-4-yl]amino]-2-phenyl-ethanol;
(2S)-2-[[2-[(1,1-dioxo-3,4-dihydro-2H-thiochromen-6-yl)amino]-5-(5-methyl-1H-pyrazol-3-yl)pyrimidin-4-yl]amino]-2-phenyl-ethanol;
(2S)-2-[[2-[(1,1-dioxo-3,4-dihydro-2H-thiochromen-6-yl)amino]-5-(5-methyloxazol-2-yl)pyrimidin-4-yl]amino]-2-phenyl-ethanol;
(2S)-2-[[2-[(1,1-dioxo-3,4-dihydro-2H-thiochromen-6-yl)amino]-5-(5-methyl-1H-1,2,4-triazol-3-yl)pyrimidin-4-yl]amino]-2-phenyl-ethanol;
2-chloro-4-[[4-[[(1S)-2-hydroxy-1-phenyl-ethyl]amino]-5-(5-methyl-1H-pyrazol-3-yl)pyrimidin-2-yl]amino]-N,N-dimethyl-benzamide;
(2S)-2-[[2-[(1,1-dioxo-3,4-dihydro-2H-thiochromen-6-yl)amino]-5-(3-methyl-1,2,4-oxadiazol-5-yl)pyrimidin-4-yl]amino]-2-phenyl-ethanol;
(2S)-2-[[2-[(1,1-dioxo-3,4-dihydro-2H-thiochromen-6-yl)amino]-5-(5-methyl-1,3,4-oxadiazol-2-yl)pyrimidin-4-yl]amino]-2-phenyl-ethanol;
(2S)-2-[[2-[(1,1-dioxo-3,4-dihydro-2H-thiochromen-6-yl)amino]-5-(5-methyl-1,2,4-oxadiazol-3-yl)pyrimidin-4-yl]amino]-2-phenyl-ethanol;
2-chloro-4-[[4-[[(1S)-2-hydroxy-1-phenyl-ethyl]amino]-5-(5-methyloxazol-2-yl)pyrimidin-2-yl]amino]-N,N-dimethyl-benzamide;
(2S)-2-[[2-[(1,1-dioxo-3,4-dihydro-2H-thiochromen-6-yl)amino]-5-(1-methyltetrazol-5-yl)pyrimidin-4-yl]amino]-2-phenyl-ethanol;
2-chloro-4-[[4-[[(1S)-2-hydroxy-1-phenyl-ethyl]amino]-5-(5-methyl-1H-1,2,4-triazol-3-yl)pyrimidin-2-yl]amino]-N,N-dimethyl-benzamide;
6-[[4-[[(1S)-2-hydroxy-1-phenyl-ethyl]amino]-5-oxazol-2-yl-pyrimidin-2-yl]amino]-1,1-dioxo-3,4-dihydro-2H-thiochromen-4-ol;
6-[[4-[[(1S)-2-hydroxy-1-phenyl-ethyl]amino]-5-(1H-triazol-5-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(1H-1,2,4-triazol-5-yl)pyrimidin-2-yl]amino]-1,1-dioxo-3,4-dihydro-2H-thiochromen-4-ol;
2-chloro-4-[[5-(5-ethyl-1,3,4-oxadiazol-2-yl)-4-[[(1S)-2-hydroxy-1-phenyl-ethyl]amino]pyrimidin-2-yl]amino]-N-methyl-benzamide;
2-chloro-4-[[5-(3-ethyl-1,2,4-oxadiazol-5-yl)-4-[[(1S)-2-hydroxy-1-phenyl-ethyl]amino]pyrimidin-2-yl]amino]-N-methyl-benzamide;
2-chloro-4-[[4-[[(1S)-2-hydroxy-1-phenyl-ethyl]amino]-5-(3-methyl-1,2,4-oxadiazol-5-yl)pyrimidin-2-yl]amino]-N,N-dimethyl-benzamide;
2-chloro-4-[[4-[[(1S)-2-hydroxy-1-phenyl-ethyl]amino]-5-(5-methyl-1,3,4-oxadiazol-2-yl)pyrimidin-2-yl]amino]-N,N-dimethyl-benzamide;
2-chloro-4-[[4-[[(1S)-2-hydroxy-1-phenyl-ethyl]amino]-5-(5-methyl-1,2,4-oxadiazol-3-yl)pyrimidin-2-yl]amino]-N,N-dimethyl-benzamide;
2-chloro-4-[[4-[[(1S)-2-hydroxy-1-phenyl-ethyl]amino]-5-(3-isopropyl-1,2,4-oxadiazol-5-yl)pyrimidin-2-yl]amino]benzamide;
2-chloro-4-[[4-[[(1S)-2-hydroxy-1-phenyl-ethyl]amino]-5-(5-isopropyl-1,3,4-oxadiazol-2-yl)pyrimidin-2-yl]amino]benzamide;
6-[[5-[5-(difluoromethyl)-1,3,4-oxadiazol-2-yl]-4-[[(1S)-2-hydroxy-1-phenyl-ethyl]amino]pyrimidin-2-yl]amino]-3,4-dihydro-2H-isoquinolin-1-one;
6-[[5-[5-(difluoromethyl)-1,3,4-oxadiazol-2-yl]-4-[[(1S)-2-hydroxy-1-phenyl-ethyl]amino]pyrimidin-2-yl]amino]-3,4-dihydro-1H-quinolin-2-one;
6-[[5-[3-(difluoromethyl)-1,2,4-oxadiazol-5-yl]-4-[[(1S)-2-hydroxy-1-phenyl-ethyl]amino]pyrimidin-2-yl]amino]-3,4-dihydro-2H-isoquinolin-1-one;
6-[[5-[3-(difluoromethyl)-1,2,4-oxadiazol-5-yl]-4-[[(1S)-2-hydroxy-1-phenyl-ethyl]amino]pyrimidin-2-yl]amino]-3,4-dihydro-1H-quinolin-2-one;
2-chloro-4-[[4-[[(1S)-2-hydroxy-1-phenyl-ethyl]amino]-5-(1-methyltetrazol-5-yl)pyrimidin-2-yl]amino]-N,N-dimethyl-benzamide;
6-[[4-[[(1S)-2-hydroxy-1-phenyl-ethyl]amino]-5-(1,3,4-oxadiazol-2-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(1,2,4-oxadiazol-5-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(1H-tetrazol-5-yl)pyrimidin-2-yl]amino]-1,1-dioxo-3,4-dihydro-2H-thiochromen-4-ol;
(2S)-2-[[5-(5-ethyl-1,3,4-oxadiazol-2-yl)-2-(3-methyl-4-methylsulfonyl-anilino)pyrimidin-4-yl]amino]-2-phenyl-ethanol;
(2S)-2-[[5-(3-ethyl-1,2,4-oxadiazol-5-yl)-2-(3-methyl-4-methylsulfonyl-anilino)pyrimidin-4-yl]amino]-2-phenyl-ethanol;
(2S)-2-[[5-(3-isopropyl-1,2,4-oxadiazol-5-yl)-2-(4-methylsulfonylanilino)-pyrimidin-4-yl]amino]-2-phenyl-ethanol;
(2S)-2-[[5-(5-isopropyl-1,3,4-oxadiazol-2-yl)-2-(4-methylsulfonylanilino)-pyrimidin-4-yl]amino]-2-phenyl-ethanol;
2-chloro-4-[[4-[[(1S)-2-hydroxy-1-phenyl-ethyl]amino]-5-(5-methyl-1,3,4-thiadiazol-2-yl)pyrimidin-2-yl]amino]-N-methyl-benzamide;
4-[[5-[5-(difluoromethyl)-1,3,4-oxadiazol-2-yl]-4-[[(1S)-2-hydroxy-1-phenyl-ethyl]amino]pyrimidin-2-yl]amino]-N,N-dimethyl-benzamide;
4-[[5-[5-(difluoromethyl)-1,3,4-oxadiazol-2-yl]-4-[[(1S)-2-hydroxy-1-phenyl-ethyl]amino]pyrimidin-2-yl]amino]-N,2-dimethyl-benzamide;
4-[[5-[3-(difluoromethyl)-1,2,4-oxadiazol-5-yl]-4-[[(1S)-2-hydroxy-1-phenyl-ethyl]amino]pyrimidin-2-yl]amino]-N,N-dimethyl-benzamide;
4-[[5-[3-(difluoromethyl)-1,2,4-oxadiazol-5-yl]-4-[[(1S)-2-hydroxy-1-phenyl-ethyl]amino]pyrimidin-2-yl]amino]-N,2-dimethyl-benzamide;
(2S)-2-[[2-(3-methyl-4-methylsulfonyl-anilino)-5-(5-methyl-1,3,4-thiadiazol-2-yl)pyrimidin-4-yl]amino]-2-phenyl-ethanol;
ethyl 4-[(3S)-3-(hydroxymethyl)-3,4-dihydro-1H-isoquinolin-2-yl]-2-(3-methyl-4-methylsulfonyl-anilino)pyrimidine-5-carboxylate;
(2S)-2-[[2-(3-chloro-4-methylsulfonyl-anilino)-5-(5-methyl-1H-pyrazol-3-yl)pyrimidin-4-yl]amino]-2-phenyl-ethanol;
(2S)-2-[[5-(5-ethyl-1,3,4-oxadiazol-2-yl)-2-(3-fluoro-4-methylsulfonyl-anilino)pyrimidin-4-yl]amino]-2-phenyl-ethanol;
(2S)-2-[[5-(3-ethyl-1,2,4-oxadiazol-5-yl)-2-(3-fluoro-4-methylsulfonyl-anilino)pyrimidin-4-yl]amino]-2-phenyl-ethanol;
4-[[4-[[(1S)-2-hydroxy-1-phenyl-ethyl]amino]-5-[3-(trifluoromethyl)-1H-1,2,4-triazol-5-yl]pyrimidin-2-yl]amino]-2-methyl-benzamide;
(2S)-2-[[2-(3-chloro-4-methylsulfonyl-anilino)-5-(5-methyloxazol-2-yl)pyrimidin-4-yl]amino]-2-phenyl-ethanol;
(2S)-2-[[2-(3-chloro-4-methylsulfonyl-anilino)-5-(5-methyl-1H-1,2,4-triazol-3-yl)pyrimidin-4-yl]amino]-2-phenyl-ethanol;
4-[[4-[[(1S)-2-hydroxy-1-phenyl-ethyl]amino]-5-[3-(trifluoromethyl)-1,2,4-oxadiazol-5-yl]pyrimidin-2-yl]amino]-2-methyl-benzamide;
4-[[4-[[(1S)-2-hydroxy-1-phenyl-ethyl]amino]-5-[5-(trifluoromethyl)-1,3,4-oxadiazol-2-yl]pyrimidin-2-yl]amino]-2-methyl-benzamide;
[2-(3-fluoro-4-methylsulfonyl-anilino)-4-[[(1S)-2-hydroxy-1-phenyl-ethyl]amino]pyrimidin-5-yl]-pyrrolidin-1-yl-methanone;
4-[[(1S)-2-hydroxy-1-phenyl-ethyl]amino]-N-(2-methoxyethyl)-2-(3-methyl-4-methylsulfonyl-anilino)pyrimidine-5-carboxamide;
6-[[5-(5-tert-butyl-1,3,4-oxadiazol-2-yl)-4-[[(1S)-2-hydroxy-1-phenyl-ethyl]amino]-pyrimidin-2-yl]amino]-3,4-dihydro-2H-isoquinolin-1-one;
6-[[5-(5-tert-butyl-1,3,4-oxadiazol-2-yl)-4-[[(1S)-2-hydroxy-1-phenyl-ethyl]amino]pyrimidin-2-yl]amino]-3,4-dihydro-1H-quinolin-2-one;
6-[[5-(3-tert-butyl-1,2,4-oxadiazol-5-yl)-4-[[(1S)-2-hydroxy-1-phenyl-ethyl]amino]pyrimidin-2-yl]amino]-3,4-dihydro-2H-isoquinolin-1-one;
6-[[5-(3-tert-butyl-1,2,4-oxadiazol-5-yl)-4-[[(1S)-2-hydroxy-1-phenyl-ethyl]amino]pyrimidin-2-yl]amino]-3,4-dihydro-1H-quinolin-2-one;
7-[[5-(5-ethyl-1,3,4-oxadiazol-2-yl)-4-[[(1S)-2-hydroxy-1-phenyl-ethyl]amino]pyrimidin-2-yl]amino]-3,3-dimethyl-2,4-dihydroisoquinolin-1-one;
7-[[5-(3-ethyl-1,2,4-oxadiazol-5-yl)-4-[[(1S)-2-hydroxy-1-phenyl-ethyl]amino]pyrimidin-2-yl]amino]-3,3-dimethyl-2,4-dihydroisoquinolin-1-one;
6-[[4-[[(1S)-2-hydroxy-1-phenyl-ethyl]amino]-5-(3-isopropyl-1,2,4-oxadiazol-5-yl)pyrimidin-2-yl]amino]-2-methyl-3,4-dihydroisoquinolin-1-one;
7-[[4-[[(1S)-2-hydroxy-1-phenyl-ethyl]amino]-5-(3-isopropyl-1,2,4-oxadiazol-5-yl)pyrimidin-2-yl]amino]-2-methyl-1,4-dihydroisoquinolin-3-one;
6-[[4-[[(1S)-2-hydroxy-1-phenyl-ethyl]amino]-5-(5-isopropyl-1,3,4-oxadiazol-2-yl)pyrimidin-2-yl]amino]-2-methyl-3,4-dihydroisoquinolin-1-one;
7-[[4-[[(1S)-2-hydroxy-1-phenyl-ethyl]amino]-5-(5-isopropyl-1,3,4-oxadiazol-2-yl)pyrimidin-2-yl]amino]-2-methyl-1,4-dihydroisoquinolin-3-one;
(2S)-2-[[2-(3-chloro-4-methylsulfonyl-anilino)-5-(5-methyl-1,2,4-oxadiazol-3-yl)pyrimidin-4-yl]amino]-2-phenyl-ethanol;
(2S)-2-[[2-(3-chloro-4-methylsulfonyl-anilino)-5-(5-methyl-1,3,4-oxadiazol-2-yl)pyrimidin-4-yl]amino]-2-phenyl-ethanol;
(2S)-2-[[2-(3-chloro-4-methylsulfonyl-anilino)-5-(3-methyl-1,2,4-oxadiazol-5-yl)pyrimidin-4-yl]amino]-2-phenyl-ethanol;
(2S)-2-[[2-(3-fluoro-4-methylsulfonyl-anilino)-5-(5-methyl-1,3,4-thiadiazol-2-yl)pyrimidin-4-yl]amino]-2-phenyl-ethanol;
(2S)-2-[[2-(3-chloro-4-methylsulfonyl-anilino)-5-(1-methyltetrazol-5-yl)pyrimidin-4-yl]amino]-2-phenyl-ethanol;
ethyl 2-(3-fluoro-4-methylsulfonyl-anilino)-4-[3-(hydroxymethyl)-3,4-dihydro-1H-isoquinolin-2-yl]pyrimidine-5-carboxylate;
N-cyclopentyl-4-[[(1S)-2-hydroxy-1-phenyl-ethyl]amino]-2-[(2-methyl-1-oxo-3,4-dihydroisoquinolin-6-yl)amino]pyrimidine-5-carboxamide;
2-chloro-4-[[5-[3-(difluoromethyl)-1,2,4-oxadiazol-5-yl]-4-[[(1S)-2-hydroxy-1-phenyl-ethyl]amino]pyrimidin-2-yl]amino]benzamide;
2-chloro-4-[[5-[5-(difluoromethyl)-1,3,4-oxadiazol-2-yl]-4-[[(1S)-2-hydroxy-1-phenyl-ethyl]amino]pyrimidin-2-yl]amino]benzamide;
7-[[4-[[(1S)-2-hydroxy-1-phenyl-ethyl]amino]-5-(5-methyl-1,3,4-thiadiazol-2-yl)pyrimidin-2-yl]amino]-3,3-dimethyl-2,4-dihydroisoquinolin-1-one;
4-[[5-(5-tert-butyl-1,3,4-oxadiazol-2-yl)-4-[[(1S)-2-hydroxy-1-phenyl-ethyl]amino]pyrimidin-2-yl]amino]-N,N-dimethyl-benzamide;
4-[[5-(5-tert-butyl-1,3,4-oxadiazol-2-yl)-4-[[(1S)-2-hydroxy-1-phenyl-ethyl]amino]pyrimidin-2-yl]amino]-N,2-dimethyl-benzamide;
4-[[5-(3-tert-butyl-1,2,4-oxadiazol-5-yl)-4-[[(1S)-2-hydroxy-1-phenyl-ethyl]amino]pyrimidin-2-yl]amino]-N,N-dimethyl-benzamide;
4-[[5-(3-tert-butyl-1,2,4-oxadiazol-5-yl)-4-[[(1S)-2-hydroxy-1-phenyl-ethyl]amino]pyrimidin-2-yl]amino]-N,2-dimethyl-benzamide;
4-[[4-[[(1S)-2-hydroxy-1-phenyl-ethyl]amino]-5-(3-isopropyl-1,2,4-oxadiazol-5-yl)pyrimidin-2-yl]amino]-N,N,2-trimethyl-benzamide;
4-[[4-[[(1S)-2-hydroxy-1-phenyl-ethyl]amino]-5-(5-isopropyl-1,3,4-oxadiazol-2-yl)pyrimidin-2-yl]amino]-N,N,2-trimethyl-benzamide;
(2S)-2-[[5-[3-(difluoromethyl)-1,2,4-oxadiazol-5-yl]-2-(4-methylsulfonylanilino)-pyrimidin-4-yl]amino]-2-phenyl-ethanol;
(2S)-2-[[5-[5-(difluoromethyl)-1,3,4-oxadiazol-2-yl]-2-(4-methylsulfonylanilino)-pyrimidin-4-yl]amino]-2-phenyl-ethanol;
2-(3-fluoro-4-methylsulfonyl-anilino)-4-[[(1S)-2-hydroxy-1-phenyl-ethyl]amino]-N-(2-methoxyethyl)-pyrimidine-5-carboxamide;
2-(3-fluoro-4-methylsulfonyl-anilino)-N-(2-hydroxyethyl)-4-[[(1S)-2-hydroxy-1-phenyl-ethyl]amino]-N-methyl-pyrimidine-5-carboxamide;
ethyl 4-[[(1S)-1-(4-chlorophenyl)-2-hydroxy-ethyl]amino]-2-(3-methyl-4-methylsulfonyl-anilino)pyrimidine-5-carboxylate;
(2S)-2-[[2-[(1,1-dioxo-2H-thiochromen-6-yl)amino]-5-(3-ethyl-1,2,4-oxadiazol-5-yl)pyrimidin-4-yl]amino]-2-phenyl-ethanol;
(2S)-2-[[2-[(1,1-dioxo-2H-thiochromen-6-yl)amino]-5-(5-ethyl-1,3,4-oxadiazol-2-yl)pyrimidin-4-yl]amino]-2-phenyl-ethanol;
(2S)-2-[[2-[(1,1-dioxo-2H-thiochromen-6-yl)amino]-5-(5-methyl-1,3,4-thiadiazol-2-yl)pyrimidin-4-yl]amino]-2-phenyl-ethanol;
6-[[4-[[(1S)-2-hydroxy-1-phenyl-ethyl]amino]-5-(5-methyl-1H-pyrazol-3-yl)pyrimidin-2-yl]amino]-1,1-dioxo-3,4-dihydro-2H-thiochromen-4-ol;
(2S)-2-[[2-[(1,1-dioxo-3,4-dihydro-2H-thiochromen-6-yl)amino]-5-(3-ethyl-1,2,4-oxadiazol-5-yl)pyrimidin-4-yl]amino]-2-phenyl-ethanol;
(2S)-2-[[2-[(1,1-dioxo-3,4-dihydro-2H-thiochromen-6-yl)amino]-5-(5-ethyl-1,3,4-oxadiazol-2-yl)pyrimidin-4-yl]amino]-2-phenyl-ethanol;
6-[[4-[[(1S)-2-hydroxy-1-phenyl-ethyl]amino]-5-(5-methyloxazol-2-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(5-methyl-1H-1,2,4-triazol-3-yl)pyrimidin-2-yl]amino]-1,1-dioxo-3,4-dihydro-2H-thiochromen-4-ol;
2-chloro-4-[[4-[[(1S)-2-hydroxy-1-phenyl-ethyl]amino]-5-(5-isopropyl-1,3,4-oxadiazol-2-yl)pyrimidin-2-yl]amino]-N-methyl-benzamide;
2-chloro-4-[[4-[[(1S)-2-hydroxy-1-phenyl-ethyl]amino]-5-(3-isopropyl-1,2,4-oxadiazol-5-yl)pyrimidin-2-yl]amino]-N-methyl-benzamide;
2-chloro-4-[[5-(3-ethyl-1,2,4-oxadiazol-5-yl)-4-[[(1S)-2-hydroxy-1-phenyl-ethyl]amino]pyrimidin-2-yl]amino]-N,N-dimethyl-benzamide;
2-chloro-4-[[5-(5-ethyl-1,3,4-oxadiazol-2-yl)-4-[[(1S)-2-hydroxy-1-phenyl-ethyl]amino]pyrimidin-2-yl]amino]-N,N-dimethyl-benzamide;
4-[[5-(3-tert-butyl-1,2,4-oxadiazol-5-yl)-4-[[(1S)-2-hydroxy-1-phenyl-ethyl]amino]pyrimidin-2-yl]amino]-2-chloro-benzamide;
4-[[5-(5-tert-butyl-1,3,4-oxadiazol-2-yl)-4-[[(1S)-2-hydroxy-1-phenyl-ethyl]amino]pyrimidin-2-yl]amino]-2-chloro-benzamide;
6-[[5-[5-(difluoromethyl)-1,3,4-oxadiazol-2-yl]-4-[[(1S)-2-hydroxy-1-phenyl-ethyl]amino]pyrimidin-2-yl]amino]-2-methyl-3,4-dihydroisoquinolin-1-one;
7-[[5-[5-(difluoromethyl)-1,3,4-oxadiazol-2-yl]-4-[[(1S)-2-hydroxy-1-phenyl-ethyl]amino]pyrimidin-2-yl]amino]-2-methyl-1,4-dihydroisoquinolin-3-one;
6-[[5-[3-(difluoromethyl)-1,2,4-oxadiazol-5-yl]-4-[[(1S)-2-hydroxy-1-phenyl-ethyl]amino]pyrimidin-2-yl]amino]-2-methyl-3,4-dihydroisoquinolin-1-one;
7-[[5-[3-(difluoromethyl)-1,2,4-oxadiazol-5-yl]-4-[[(1S)-2-hydroxy-1-phenyl-ethyl]amino]pyrimidin-2-yl]amino]-2-methyl-1,4-dihydroisoquinolin-3-one;
(2S)-2-[[2-[(1,1-dioxo-3,4-dihydro-2H-thiochromen-6-yl)amino]-5-(5-methyl-1,3,4-thiadiazol-2-yl)pyrimidin-4-yl]amino]-2-phenyl-ethanol;
6-[[4-[[(1S)-2-hydroxy-1-phenyl-ethyl]amino]-5-(5-methyl-1,2,4-oxadiazol-3-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(5-methyl-1,3,4-oxadiazol-2-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(3-methyl-1,2,4-oxadiazol-5-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(1-methyltetrazol-5-yl)pyrimidin-2-yl]amino]-1,1-dioxo-3,4-dihydro-2H-thiochromen-4-ol;
(2S)-2-[[5-(5-isopropyl-1,3,4-oxadiazol-2-yl)-2-(3-methyl-4-methylsulfonyl-anilino)pyrimidin-4-yl]amino]-2-phenyl-ethanol;
(2S)-2-[[5-(3-isopropyl-1,2,4-oxadiazol-5-yl)-2-(3-methyl-4-methylsulfonyl-anilino)pyrimidin-4-yl]amino]-2-phenyl-ethanol;
(2S)-2-[[5-(3-tert-butyl-1,2,4-oxadiazol-5-yl)-2-(4-methylsulfonylanilino)-pyrimidin-4-yl]amino]-2-phenyl-ethanol;
(2S)-2-[[5-(5-tert-butyl-1,3,4-oxadiazol-2-yl)-2-(4-methylsulfonylanilino)-pyrimidin-4-yl]amino]-2-phenyl-ethanol;
2-chloro-4-[[4-[[(1S)-2-hydroxy-1-phenyl-ethyl]amino]-5-(5-methyl-1,3,4-thiadiazol-2-yl)pyrimidin-2-yl]amino]-N,N-dimethyl-benzamide;
4-[[5-[5-(difluoromethyl)-1,3,4-oxadiazol-2-yl]-4-[[(1S)-2-hydroxy-1-phenyl-ethyl]amino]pyrimidin-2-yl]amino]-N,N,2-trimethyl-benzamide;
4-[[5-[3-(difluoromethyl)-1,2,4-oxadiazol-5-yl]-4-[[(1S)-2-hydroxy-1-phenyl-ethyl]amino]pyrimidin-2-yl]amino]-N,N,2-trimethyl-benzamide;
N-cyclopentyl-4-[[(1S)-2-hydroxy-1-phenyl-ethyl]amino]-2-(3-methyl-4-methylsulfonyl-anilino)pyrimidine-5-carboxamide;
6-[[4-[[(1S)-2-hydroxy-1-phenyl-ethyl]amino]-5-[3-(trifluoromethyl)-1H-1,2,4-triazol-5-yl]pyrimidin-2-yl]amino]-3,4-dihydro-1H-quinolin-2-one;
6-[[4-[[(1S)-2-hydroxy-1-phenyl-ethyl]amino]-5-[3-(trifluoromethyl)-1,2,4-oxadiazol-5-yl]pyrimidin-2-yl]amino]-3,4-dihydro-1H-quinolin-2-one;
6-[[4-[[(1S)-2-hydroxy-1-phenyl-ethyl]amino]-5-[3-(trifluoromethyl)-1,2,4-oxadiazol-5-yl]pyrimidin-2-yl]amino]-3,4-dihydro-2H-isoquinolin-1-one;
6-[[4-[[(1S)-2-hydroxy-1-phenyl-ethyl]amino]-5-[5-(trifluoromethyl)-1,3,4-oxadiazol-2-yl]pyrimidin-2-yl]amino]-3,4-dihydro-1H-quinolin-2-one;
6-[[4-[[(1S)-2-hydroxy-1-phenyl-ethyl]amino]-5-[5-(trifluoromethyl)-1,3,4-oxadiazol-2-yl]pyrimidin-2-yl]amino]-3,4-dihydro-2H-isoquinolin-1-one;
N-cyclopentyl-2-(4-fluoro-3-phenyl-anilino)-4-[[(1S)-2-hydroxy-1-phenyl-ethyl]amino]pyrimidine-5-carboxamide;
(2S)-2-[[2-(3-fluoro-4-methylsulfonyl-anilino)-5-(5-isopropyl-1,3,4-oxadiazol-2-yl)pyrimidin-4-yl]amino]-2-phenyl-ethanol;
(2S)-2-[[2-(3-fluoro-4-methylsulfonyl-anilino)-5-(3-isopropyl-1,2,4-oxadiazol-5-yl)pyrimidin-4-yl]amino]-2-phenyl-ethanol;
4-[[4-[[(1S)-2-hydroxy-1-phenyl-ethyl]amino]-5-[3-(trifluoromethyl)-1H-1,2,4-triazol-5-yl]pyrimidin-2-yl]amino]-N,2-dimethyl-benzamide;
4-[[4-[[(1S)-2-hydroxy-1-phenyl-ethyl]amino]-5-[3-(trifluoromethyl)-1H-1,2,4-triazol-5-yl]pyrimidin-2-yl]amino]-N,N-dimethyl-benzamide;
4-[[4-[[(1S)-2-hydroxy-1-phenyl-ethyl]amino]-5-[3-(trifluoromethyl)-1,2,4-oxadiazol-5-yl]pyrimidin-2-yl]amino]-N,2-dimethyl-benzamide;
4-[[4-[[(1S)-2-hydroxy-1-phenyl-ethyl]amino]-5-[3-(trifluoromethyl)-1,2,4-oxadiazol-5-yl]pyrimidin-2-yl]amino]-N,N-dimethyl-benzamide;
4-[[4-[[(1S)-2-hydroxy-1-phenyl-ethyl]amino]-5-[5-(trifluoromethyl)-1,3,4-oxadiazol-2-yl]pyrimidin-2-yl]amino]-N,2-dimethyl-benzamide;
4-[[4-[[(1S)-2-hydroxy-1-phenyl-ethyl]amino]-5-[5-(trifluoromethyl)-1,3,4-oxadiazol-2-yl]pyrimidin-2-yl]amino]-N,N-dimethyl-benzamide;
7-[[4-[[(1S)-2-hydroxy-1-phenyl-ethyl]amino]-5-(5-isopropyl-1,3,4-oxadiazol-2-yl)pyrimidin-2-yl]amino]-3,3-dimethyl-2,4-dihydroisoquinolin-1-one;
7-[[4-[[(1S)-2-hydroxy-1-phenyl-ethyl]amino]-5-(3-isopropyl-1,2,4-oxadiazol-5-yl)pyrimidin-2-yl]amino]-3,3-dimethyl-2,4-dihydroisoquinolin-1-one;
6-[[5-(3-tert-butyl-1,2,4-oxadiazol-5-yl)-4-[[(1S)-2-hydroxy-1-phenyl-ethyl]amino]pyrimidin-2-yl]amino]-2-methyl-3,4-dihydroisoquinolin-1-one;
7-[[5-(3-tert-butyl-1,2,4-oxadiazol-5-yl)-4-[[(1S)-2-hydroxy-1-phenyl-ethyl]amino]pyrimidin-2-yl]amino]-2-methyl-1,4-dihydroisoquinolin-3-one;
6-[[5-(5-tert-butyl-1,3,4-oxadiazol-2-yl)-4-[[(1S)-2-hydroxy-1-phenyl-ethyl]amino]pyrimidin-2-yl]amino]-2-methyl-3,4-dihydroisoquinolin-1-one;
7-[[5-(5-tert-butyl-1,3,4-oxadiazol-2-yl)-4-[[(1S)-2-hydroxy-1-phenyl-ethyl]amino]pyrimidin-2-yl]amino]-2-methyl-1,4-dihydroisoquinolin-3-one;
(2S)-2-[[2-(3-chloro-4-methylsulfonyl-anilino)-5-(5-ethyl-1,3,4-oxadiazol-2-yl)pyrimidin-4-yl]amino]-2-phenyl-ethanol;
(2S)-2-[[2-(3-chloro-4-methylsulfonyl-anilino)-5-(3-ethyl-1,2,4-oxadiazol-5-yl)pyrimidin-4-yl]amino]-2-phenyl-ethanol;
2-chloro-4-[[5-[5-(difluoromethyl)-1,3,4-oxadiazol-2-yl]-4-[[(1S)-2-hydroxy-1-phenyl-ethyl]amino]pyrimidin-2-yl]amino]-N-methyl-benzamide;
2-chloro-4-[[5-[3-(difluoromethyl)-1,2,4-oxadiazol-5-yl]-4-[[(1S)-2-hydroxy-1-phenyl-ethyl]amino]pyrimidin-2-yl]amino]-N-methyl-benzamide;
4-[[5-(3-tert-butyl-1,2,4-oxadiazol-5-yl)-4-[[(1S)-2-hydroxy-1-phenyl-ethyl]amino]pyrimidin-2-yl]amino]-N,N,2-trimethyl-benzamide;
4-[[5-(5-tert-butyl-1,3,4-oxadiazol-2-yl)-4-[[(1S)-2-hydroxy-1-phenyl-ethyl]amino]pyrimidin-2-yl]amino]-N,N,2-trimethyl-benzamide;
(2S)-2-[[2-(3-chloro-4-methylsulfonyl-anilino)-5-(5-methyl-1,3,4-thiadiazol-2-yl)pyrimidin-4-yl]amino]-2-phenyl-ethanol;
(2S)-2-[[5-[5-(difluoromethyl)-1,3,4-oxadiazol-2-yl]-2-(3-methyl-4-methylsulfonyl-anilino)pyrimidin-4-yl]amino]-2-phenyl-ethanol;
(2S)-2-[[5-[3-(difluoromethyl)-1,2,4-oxadiazol-5-yl]-2-(3-methyl-4-methylsulfonyl-anilino)pyrimidin-4-yl]amino]-2-phenyl-ethanol;
2-chloro-4-[[4-[[(1S)-2-hydroxy-1-phenyl-ethyl]amino]-5-[3-(trifluoromethyl)-1H-1,2,4-triazol-5-yl]pyrimidin-2-yl]amino]benzamide;
(2S)-2-[[2-[(1,1-dioxo-2H-thiochromen-6-yl)amino]-5-(3-isopropyl-1,2,4-oxadiazol-5-yl)pyrimidin-4-yl]amino]-2-phenyl-ethanol;
(2S)-2-[[2-[(1,1-dioxo-2H-thiochromen-6-yl)amino]-5-(5-isopropyl-1,3,4-oxadiazol-2-yl)pyrimidin-4-yl]amino]-2-phenyl-ethanol;
ethyl 4-[[(1S)-2-hydroxy-1-phenyl-ethyl]amino]-2-[(3-isopropyl-2,5-dioxo-3,4-dihydro-1H-1,4-benzodiazepin-7-yl)amino]pyrimidine-5-carboxylate;
2-chloro-4-[[4-[[(1S)-2-hydroxy-1-phenyl-ethyl]amino]-5-[3-(trifluoromethyl)-1,2,4-oxadiazol-5-yl]pyrimidin-2-yl]amino]benzamide;
2-chloro-4-[[4-[[(1S)-2-hydroxy-1-phenyl-ethyl]amino]-5-[5-(trifluoromethyl)-1,3,4-oxadiazol-2-yl]pyrimidin-2-yl]amino]benzamide;
4-[[(1S)-2-hydroxy-1-phenyl-ethyl]amino]-2-(3-methyl-4-methylsulfonyl-anilino)-N-methylsulfonyl-pyrimidine-5-carboxamide;
(2S)-2-[[2-(4-methylsulfonylanilino)-5-[3-(trifluoromethyl)-1H-1,2,4-triazol-5-yl]pyrimidin-4-yl]amino]-2-phenyl-ethanol;
2-[3-chloro-4-(trifluoromethyl)anilino]-N-cyclopentyl-4-[[(1S)-2-hydroxy-1-phenyl-ethyl]amino]-pyrimidine-5-carboxamide;
(2S)-2-[[5-[5-(difluoromethyl)-1,3,4-oxadiazol-2-yl]-2-(3-fluoro-4-methylsulfonyl-anilino)pyrimidin-4-yl]amino]-2-phenyl-ethanol;
(2S)-2-[[5-[3-(difluoromethyl)-1,2,4-oxadiazol-5-yl]-2-(3-fluoro-4-methylsulfonyl-anilino)pyrimidin-4-yl]amino]-2-phenyl-ethanol;
(2S)-2-[[2-(4-methylsulfonylanilino)-5-[3-(trifluoromethyl)-1,2,4-oxadiazol-5-yl]pyrimidin-4-yl]amino]-2-phenyl-ethanol;
(2S)-2-[[2-(4-methylsulfonylanilino)-5-[5-(trifluoromethyl)-1,3,4-oxadiazol-2-yl]pyrimidin-4-yl]amino]-2-phenyl-ethanol;
(2S)-2-[[2-[(1,1-dioxo-3,4-dihydro-2H-thiochromen-6-yl)amino]-5-(3-isopropyl-1,2,4-oxadiazol-5-yl)pyrimidin-4-yl]amino]-2-phenyl-ethanol;
(2S)-2-[[2-[(1,1-dioxo-3,4-dihydro-2H-thiochromen-6-yl)amino]-5-(5-isopropyl-1,3,4-oxadiazol-2-yl)pyrimidin-4-yl]amino]-2-phenyl-ethanol;
4-[[5-(5-tert-butyl-1,3,4-oxadiazol-2-yl)-4-[[(1S)-2-hydroxy-1-phenyl-ethyl]amino]pyrimidin-2-yl]amino]-2-chloro-N-methyl-benzamide;
4-[[5-(3-tert-butyl-1,2,4-oxadiazol-5-yl)-4-[[(1S)-2-hydroxy-1-phenyl-ethyl]amino]pyrimidin-2-yl]amino]-2-chloro-N-methyl-benzamide;
2-chloro-4-[[4-[[(1S)-2-hydroxy-1-phenyl-ethyl]amino]-5-(3-isopropyl-1,2,4-oxadiazol-5-yl)pyrimidin-2-yl]amino]-N,N-dimethyl-benzamide;
2-chloro-4-[[4-[[(1S)-2-hydroxy-1-phenyl-ethyl]amino]-5-(5-isopropyl-1,3,4-oxadiazol-2-yl)pyrimidin-2-yl]amino]-N,N-dimethyl-benzamide;
7-[[5-[5-(difluoromethyl)-1,3,4-oxadiazol-2-yl]-4-[[(1S)-2-hydroxy-1-phenyl-ethyl]amino]pyrimidin-2-yl]amino]-3,3-dimethyl-2,4-dihydroisoquinolin-1-one;
7-[[5-[3-(difluoromethyl)-1,2,4-oxadiazol-5-yl]-4-[[(1S)-2-hydroxy-1-phenyl-ethyl]amino]pyrimidin-2-yl]amino]-3,3-dimethyl-2,4-dihydroisoquinolin-1-one;
6-[[5-(5-ethyl-1,3,4-oxadiazol-2-yl)-4-[[(1S)-2-hydroxy-1-phenyl-ethyl]amino]pyrimidin-2-yl]amino]-1,1-dioxo-3,4-dihydro-2H-thiochromen-4-ol;
6-[[5-(3-ethyl-1,2,4-oxadiazol-5-yl)-4-[[(1S)-2-hydroxy-1-phenyl-ethyl]amino]pyrimidin-2-yl]amino]-1,1-dioxo-3,4-dihydro-2H-thiochromen-4-ol;
(2S)-2-[[5-(5-tert-butyl-1,3,4-oxadiazol-2-yl)-2-(3-methyl-4-methylsulfonyl-anilino)pyrimidin-4-yl]amino]-2-phenyl-ethanol;
(2S)-2-[[5-(3-tert-butyl-1,2,4-oxadiazol-5-yl)-2-(3-methyl-4-methylsulfonyl-anilino)pyrimidin-4-yl]amino]-2-phenyl-ethanol;
2-(3-fluoro-4-methylsulfonyl-anilino)-4-[[(1S)-2-hydroxy-1-phenyl-ethyl]amino]-N-methylsulfonyl-pyrimidine-5-carboxamide;
4-[[(1S)-2-hydroxy-1-phenyl-ethyl]amino]-2-(3-methyl-4-methylsulfonyl-anilino)-N-(2,2,2-trifluoroethyl)-pyrimidine-5-carboxamide;
ethyl 4-[[(1S)-1-(hydroxymethyl)-2-(1H-indol-3-yl)ethyl]amino]-2-(3-methyl-4-methylsulfonyl-anilino)pyrimidine-5-carboxylate;
6-[[4-[[(1S)-2-hydroxy-1-phenyl-ethyl]amino]-5-(5-methyl-1,3,4-thiadiazol-2-yl)pyrimidin-2-yl]amino]-1,1-dioxo-3,4-dihydro-2H-thiochromen-4-ol;
7-[[4-[[(1S)-2-hydroxy-1-phenyl-ethyl]amino]-5-[3-(trifluoromethyl)-1H-1,2,4-triazol-5-yl]pyrimidin-2-yl]amino]-2-methyl-1,4-dihydro-isoquinolin-3-one;
6-[[4-[[(1S)-2-hydroxy-1-phenyl-ethyl]amino]-5-[3-(trifluoromethyl)-1H-1,2,4-triazol-5-yl]pyrimidin-2-yl]amino]-2-methyl-3,4-dihydro-isoquinolin-1-one;
7-[[4-[[(1S)-2-hydroxy-1-phenyl-ethyl]amino]-5-[3-(trifluoromethyl)-1,2,4-oxadiazol-5-yl]pyrimidin-2-yl]amino]-2-methyl-1,4-dihydro-isoquinolin-3-one;
6-[[4-[[(1S)-2-hydroxy-1-phenyl-ethyl]amino]-5-[3-(trifluoromethyl)-1,2,4-oxadiazol-5-yl]pyrimidin-2-yl]amino]-2-methyl-3,4-dihydro-isoquinolin-1-one;
7-[[4-[[(1S)-2-hydroxy-1-phenyl-ethyl]amino]-5-[5-(trifluoromethyl)-1,3,4-oxadiazol-2-yl]pyrimidin-2-yl]amino]-2-methyl-1,4-dihydroisoquinolin-3-one;
6-[[4-[[(1S)-2-hydroxy-1-phenyl-ethyl]amino]-5-[5-(trifluoromethyl)-1,3,4-oxadiazol-2-yl]pyrimidin-2-yl]amino]-2-methyl-3,4-dihydroisoquinolin-1-one;
2-(4-fluoro-3-phenyl-anilino)-4-[[(1S)-2-hydroxy-1-phenyl-ethyl]amino]-N-(2,2,2-trifluoroethyl)-pyrimidine-5-carboxamide;
(2S)-2-[[5-[3-(difluoromethyl)-1,2,4-oxadiazol-5-yl]-2-[(1,1-dioxo-2H-thiochromen-6-yl)amino]pyrimidin-4-yl]amino]-2-phenyl-ethanol;
(2S)-2-[[5-[5-(difluoromethyl)-1,3,4-oxadiazol-2-yl]-2-[(1,1-dioxo-2H-thiochromen-6-yl)amino]pyrimidin-4-yl]amino]-2-phenyl-ethanol;
(2S)-2-[[5-(5-tert-butyl-1,3,4-oxadiazol-2-yl)-2-(3-fluoro-4-methylsulfonyl-anilino)pyrimidin-4-yl]amino]-2-phenyl-ethanol;
(2S)-2-[[5-(3-tert-butyl-1,2,4-oxadiazol-5-yl)-2-(3-fluoro-4-methylsulfonyl-anilino)pyrimidin-4-yl]amino]-2-phenyl-ethanol;
4-[[4-[[(1S)-2-hydroxy-1-phenyl-ethyl]amino]-5-[3-(trifluoromethyl)-1H-1,2,4-triazol-5-yl]pyrimidin-2-yl]amino]-N,N,2-trimethyl-benzamide;
4-[[4-[[(1S)-2-hydroxy-1-phenyl-ethyl]amino]-5-[3-(trifluoromethyl)-1,2,4-oxadiazol-5-yl]pyrimidin-2-yl]amino]-N,N,2-trimethyl-benzamide;
4-[[4-[[(1S)-2-hydroxy-1-phenyl-ethyl]amino]-5-[5-(trifluoromethyl)-1,3,4-oxadiazol-2-yl]pyrimidin-2-yl]amino]-N,N,2-trimethyl-benzamide;
7-[[5-(5-tert-butyl-1,3,4-oxadiazol-2-yl)-4-[[(1S)-2-hydroxy-1-phenyl-ethyl]amino]pyrimidin-2-yl]amino]-3,3-dimethyl-2,4-dihydroisoquinolin-1-one;
7-[[5-(3-tert-butyl-1,2,4-oxadiazol-5-yl)-4-[[(1S)-2-hydroxy-1-phenyl-ethyl]amino]pyrimidin-2-yl]amino]-3,3-dimethyl-2,4-dihydroisoquinolin-1-one;
(2S)-2-[[2-(3-chloro-4-methylsulfonyl-anilino)-5-(5-isopropyl-1,3,4-oxadiazol-2-yl)pyrimidin-4-yl]amino]-2-phenyl-ethanol;
(2S)-2-[[2-(3-chloro-4-methylsulfonyl-anilino)-5-(3-isopropyl-1,2,4-oxadiazol-5-yl)pyrimidin-4-yl]amino]-2-phenyl-ethanol;
(2S)-2-[[5-[3-(difluoromethyl)-1,2,4-oxadiazol-5-yl]-2-[(1,1-dioxo-3,4-dihydro-2H-thiochromen-6-yl)amino]pyrimidin-4-yl]amino]-2-phenyl-ethanol;
(2S)-2-[[5-[5-(difluoromethyl)-1,3,4-oxadiazol-2-yl]-2-[(1,1-dioxo-3,4-dihydro-2H-thiochromen-6-yl)amino]pyrimidin-4-yl]amino]-2-phenyl-ethanol;
2-chloro-4-[[5-[3-(difluoromethyl)-1,2,4-oxadiazol-5-yl]-4-[[(1S)-2-hydroxy-1-phenyl-ethyl]amino]pyrimidin-2-yl]amino]-N,N-dimethyl-benzamide;
2-chloro-4-[[5-[5-(difluoromethyl)-1,3,4-oxadiazol-2-yl]-4-[[(1S)-2-hydroxy-1-phenyl-ethyl]amino]pyrimidin-2-yl]amino]-N,N-dimethyl-benzamide;
2-chloro-4-[[4-[[(1S)-2-hydroxy-1-phenyl-ethyl]amino]-5-[3-(trifluoromethyl)-1H-1,2,4-triazol-5-yl]pyrimidin-2-yl]amino]-N-methyl-benzamide;
(2S)-2-[[5-(3-tert-butyl-1,2,4-oxadiazol-5-yl)-2-[(1,1-dioxo-2H-thiochromen-6-yl)amino]pyrimidin-4-yl]amino]-2-phenyl-ethanol;
(2S)-2-[[5-(5-tert-butyl-1,3,4-oxadiazol-2-yl)-2-[(1,1-dioxo-2H-thiochromen-6-yl)amino]pyrimidin-4-yl]amino]-2-phenyl-ethanol;
2-chloro-4-[[4-[[(1S)-2-hydroxy-1-phenyl-ethyl]amino]-5-[5-(trifluoromethyl)-1,3,4-oxadiazol-2-yl]pyrimidin-2-yl]amino]-N-methyl-benzamide;
2-chloro-4-[[4-[[(1S)-2-hydroxy-1-phenyl-ethyl]amino]-5-[3-(trifluoromethyl)-1,2,4-oxadiazol-5-yl]pyrimidin-2-yl]amino]-N-methyl-benzamide;
(2S)-2-[[2-(3-methyl-4-methylsulfonyl-anilino)-5-[3-(trifluoromethyl)-1H-1,2,4-triazol-5-yl]pyrimidin-4-yl]amino]-2-phenyl-ethanol;
(2S)-2-[[2-(3-methyl-4-methylsulfonyl-anilino)-5-[5-(trifluoromethyl)-1,3,4-oxadiazol-2-yl]pyrimidin-4-yl]amino]-2-phenyl-ethanol;
(2S)-2-[[2-(3-methyl-4-methylsulfonyl-anilino)-5-[3-(trifluoromethyl)-1,2,4-oxadiazol-5-yl]pyrimidin-4-yl]amino]-2-phenyl-ethanol;
(2S)-2-[[5-(3-tert-butyl-1,2,4-oxadiazol-5-yl)-2-[(1,1-dioxo-3,4-dihydro-2H-thiochromen-6-yl)amino]pyrimidin-4-yl]amino]-2-phenyl-ethanol;
(2S)-2-[[5-(5-tert-butyl-1,3,4-oxadiazol-2-yl)-2-[(1,1-dioxo-3,4-dihydro-2H-thiochromen-6-yl)amino]pyrimidin-4-yl]amino]-2-phenyl-ethanol;
4-[[5-(3-tert-butyl-1,2,4-oxadiazol-5-yl)-4-[[(1S)-2-hydroxy-1-phenyl-ethyl]amino]pyrimidin-2-yl]amino]-2-chloro-N,N-dimethyl-benzamide;
4-[[5-(5-tert-butyl-1,3,4-oxadiazol-2-yl)-4-[[(1S)-2-hydroxy-1-phenyl-ethyl]amino]pyrimidin-2-yl]amino]-2-chloro-N,N-dimethyl-benzamide;
(2S)-2-[[2-(3-chloro-4-methylsulfonyl-anilino)-5-[5-(difluoromethyl)-1,3,4-oxadiazol-2-yl]pyrimidin-4-yl]amino]-2-phenyl-ethanol;
(2S)-2-[[2-(3-chloro-4-methylsulfonyl-anilino)-5-[3-(difluoromethyl)-1,2,4-oxadiazol-5-yl]pyrimidin-4-yl]amino]-2-phenyl-ethanol;
6-[[4-[[(1S)-2-hydroxy-1-phenyl-ethyl]amino]-5-(5-isopropyl-1,3,4-oxadiazol-2-yl)pyrimidin-2-yl]amino]-1,1-dioxo-3,4-dihydro-2H-thiochromen-4-ol;
6-[[4-[[(1S)-2-hydroxy-1-phenyl-ethyl]amino]-5-(3-isopropyl-1,2,4-oxadiazol-5-yl)pyrimidin-2-yl]amino]-1,1-dioxo-3,4-dihydro-2H-thiochromen-4-ol;
(2S)-2-[[2-(3-fluoro-4-methylsulfonyl-anilino)-5-[3-(trifluoromethyl)-1H-1,2,4-triazol-5-yl]pyrimidin-4-yl]amino]-2-phenyl-ethanol;
(2S)-2-[[2-(3-fluoro-4-methylsulfonyl-anilino)-5-[5-(trifluoromethyl)-1,3,4-oxadiazol-2-yl]pyrimidin-4-yl]amino]-2-phenyl-ethanol;
(2S)-2-[[2-(3-fluoro-4-methylsulfonyl-anilino)-5-[3-(trifluoromethyl)-1,2,4-oxadiazol-5-yl]pyrimidin-4-yl]amino]-2-phenyl-ethanol;
7-[[4-[[(1S)-2-hydroxy-1-phenyl-ethyl]amino]-5-[3-(trifluoromethyl)-1H-1,2,4-triazol-5-yl]pyrimidin-2-yl]amino]-3,3-dimethyl-2,4-dihydroisoquinolin-1-one;
7-[[4-[[(1S)-2-hydroxy-1-phenyl-ethyl]amino]-5-[3-(trifluoromethyl)-1,2,4-oxadiazol-5-yl]pyrimidin-2-yl]amino]-3,3-dimethyl-2,4-dihydroisoquinolin-1-one;
7-[[4-[[(1S)-2-hydroxy-1-phenyl-ethyl]amino]-5-[5-(trifluoromethyl)-1,3,4-oxadiazol-2-yl]pyrimidin-2-yl]amino]-3,3-dimethyl-2,4-dihydroisoquinolin-1-one;
(2S)-2-[[5-(5-tert-butyl-1,3,4-oxadiazol-2-yl)-2-(3-chloro-4-methylsulfonyl-anilino)pyrimidin-4-yl]amino]-2-phenyl-ethanol;
(2S)-2-[[5-(3-tert-butyl-1,2,4-oxadiazol-5-yl)-2-(3-chloro-4-methylsulfonyl-anilino)pyrimidin-4-yl]amino]-2-phenyl-ethanol;
(2S)-2-[[2-[(1,1-dioxo-2H-thiochromen-6-yl)amino]-5-[3-(trifluoromethyl)-1H-1,2,4-triazol-5-yl]pyrimidin-4-yl]amino]-2-phenyl-ethanol;
(2S)-2-[[2-[(1,1-dioxo-2H-thiochromen-6-yl)amino]-5-[3-(trifluoromethyl)-1,2,4-oxadiazol-5-yl]pyrimidin-4-yl]amino]-2-phenyl-ethanol;
(2S)-2-[[2-[(1,1-dioxo-2H-thiochromen-6-yl)amino]-5-[5-(trifluoromethyl)-1,3,4-oxadiazol-2-yl]pyrimidin-4-yl]amino]-2-phenyl-ethanol;
6-[[5-[5-(difluoromethyl)-1,3,4-oxadiazol-2-yl]-4-[[(1S)-2-hydroxy-1-phenyl-ethyl]amino]pyrimidin-2-yl]amino]-1,1-dioxo-3,4-dihydro-2H-thiochromen-4-ol;
6-[[5-[3-(difluoromethyl)-1,2,4-oxadiazol-5-yl]-4-[[(1S)-2-hydroxy-1-phenyl-ethyl]amino]pyrimidin-2-yl]amino]-1,1-dioxo-3,4-dihydro-2H-thiochromen-4-ol;
(2S)-2-[[2-[(1,1-dioxo-3,4-dihydro-2H-thiochromen-6-yl)amino]-5-[3-(trifluoromethyl)-1H-1,2,4-triazol-5-yl]pyrimidin-4-yl]amino]-2-phenyl-ethanol;
(2S)-2-[[2-[(1,1-dioxo-3,4-dihydro-2H-thiochromen-6-yl)amino]-5-[3-(trifluoromethyl)-1,2,4-oxadiazol-5-yl]pyrimidin-4-yl]amino]-2-phenyl-ethanol;
(2S)-2-[[2-[(1,1-dioxo-3,4-dihydro-2H-thiochromen-6-yl)amino]-5-[5-(trifluoromethyl)-1,3,4-oxadiazol-2-yl]pyrimidin-4-yl]amino]-2-phenyl-ethanol;
2-chloro-4-[[4-[[(1S)-2-hydroxy-1-phenyl-ethyl]amino]-5-[3-(trifluoromethyl)-1H-1,2,4-triazol-5-yl]pyrimidin-2-yl]amino]-N,N-dimethyl-benzamide;
2-chloro-4-[[4-[[(1S)-2-hydroxy-1-phenyl-ethyl]amino]-5-[3-(trifluoromethyl)-1,2,4-oxadiazol-5-yl]pyrimidin-2-yl]amino]-N,N-dimethyl-benzamide;
2-chloro-4-[[4-[[(1S)-2-hydroxy-1-phenyl-ethyl]amino]-5-[5-(trifluoromethyl)-1,3,4-oxadiazol-2-yl]pyrimidin-2-yl]amino]-N,N-dimethyl-benzamide;
6-[[5-(5-tert-butyl-1,3,4-oxadiazol-2-yl)-4-[[(1S)-2-hydroxy-1-phenyl-ethyl]amino]pyrimidin-2-yl]amino]-1,1-dioxo-3,4-dihydro-2H-thiochromen-4-ol;
6-[[5-(3-tert-butyl-1,2,4-oxadiazol-5-yl)-4-[[(1S)-2-hydroxy-1-phenyl-ethyl]amino]pyrimidin-2-yl]amino]-1,1-dioxo-3,4-dihydro-2H-thiochromen-4-ol;
(2S)-2-[[2-(3-chloro-4-methylsulfonyl-anilino)-5-[3-(trifluoromethyl)-1H-1,2,4-triazol-5-yl]pyrimidin-4-yl]amino]-2-phenyl-ethanol;
(2S)-2-[[2-(3-chloro-4-methylsulfonyl-anilino)-5-[5-(trifluoromethyl)-1,3,4-oxadiazol-2-yl]pyrimidin-4-yl]amino]-2-phenyl-ethanol;
(2S)-2-[[2-(3-chloro-4-methylsulfonyl-anilino)-5-[3-(trifluoromethyl)-1,2,4-oxadiazol-5-yl]pyrimidin-4-yl]amino]-2-phenyl-ethanol;
6-[[4-[[(1S)-2-hydroxy-1-phenyl-ethyl]amino]-5-[3-(trifluoromethyl)-1H-1,2,4-triazol-5-yl]pyrimidin-2-yl]amino]-1,1-dioxo-3,4-dihydro-2H-thiochromen-4-ol;
6-[[4-[[(1S)-2-hydroxy-1-phenyl-ethyl]amino]-5-[5-(trifluoromethyl)-1,3,4-oxadiazol-2-yl]pyrimidin-2-yl]amino]-1,1-dioxo-3,4-dihydro-2H-thiochromen-4-ol;
6-[[4-[[(1S)-2-hydroxy-1-phenyl-ethyl]amino]-5-[3-(trifluoromethyl)-1,2,4-oxadiazol-5-yl]pyrimidin-2-yl]amino]-1,1-dioxo-3,4-dihydro-2H-thiochromen-4-ol;
6-[[4-[[(1S)-2-hydroxy-1-phenyl-ethyl]amino]-5-(3-methyl-1,2,4-oxadiazol-5-yl)pyrimidin-2-yl]amino]-3,4-dihydro-2H-isoquinolin-1-one;
ethyl 2-(4-carbamoyl-3-chloro-anilino)-4-[[(1S)-2-hydroxy-1-phenyl-ethyl]amino]pyrimidine-5-carboxylate;
ethyl 2-[3-chloro-4-(methylcarbamoyl)anilino]-4-[[(1S)-2-hydroxy-1-phenyl-ethyl]amino]pyrimidine-5-carboxylate;
ethyl 6-[5-[4-[[(1S)-2-hydroxy-1-phenyl-ethyl]amino]-2-(3-methyl-4-methylsulfonyl-anilino)pyrimidin-5-yl]-1,3,4-oxadiazol-2-yl]hexanoate;
ethyl 6-[5-[2-(3-fluoro-4-methylsulfonyl-anilino)-4-[[(1S)-2-hydroxy-1-phenyl-ethyl]amino]pyrimidin-5-yl]-1,3,4-oxadiazol-2-yl]hexanoate;
6-[[4-[[(1S)-2-hydroxy-1-phenyl-ethyl]amino]-5-(1,3,4-oxadiazol-2-yl)pyrimidin-2-yl]amino]-3,4-dihydro-2H-isoquinolin-1-one;
6-[[4-[[(1S)-2-hydroxy-1-phenyl-ethyl]amino]-5-methylsulfonyl-pyrimidin-2-yl]amino]-3,4-dihydro-2H-isoquinolin-1-one;
ethyl 4-[[(1S)-2-hydroxy-1-phenyl-ethyl]amino]-2-[(1-oxoisoindolin-5-yl)amino]pyrimidine-5-carboxylate;
ethyl 4-[[(1S)-2-hydroxy-1-phenyl-ethyl]amino]-2-[(1-oxo-2,3,4,5-tetrahydro-2-benzazepin-7-yl)amino]pyrimidine-5-carboxylate;
4-[[(1S)-2-hydroxy-1-phenyl-ethyl]amino]-2-[(1-oxo-3,4-dihydro-2H-isoquinolin-6-yl)amino]pyrimidine-5-carbonitrile;
ethyl 4-[[(1R)-2-hydroxy-1-phenyl-ethyl]amino]-2-[(1-oxo-3,4-dihydro-2H-isoquinolin-6-yl)amino]pyrimidine-5-carboxylate;
ethyl 4-[[(1S)-2-hydroxy-1-(4-methoxyphenyl)ethyl]amino]-2-[(1-oxo-3,4-dihydro-2H-isoquinolin-6-yl)amino]pyrimidine-5-carboxylate;
ethyl 4-[[(1S)-2-hydroxy-1-(3-methoxyphenyl)ethyl]amino]-2-[(1-oxo-3,4-dihydro-2H-isoquinolin-6-yl)amino]pyrimidine-5-carboxylate;
5-[[4-[[(1S)-2-hydroxy-1-phenyl-ethyl]amino]-5-(3-methyl-1,2,4-oxadiazol-5-yl)pyrimidin-2-yl]amino]isoindolin-1-one;
5-[[4-[[(1S)-2-hydroxy-1-phenyl-ethyl]amino]-5-(1,3,4-oxadiazol-2-yl)pyrimidin-2-yl]amino]isoindolin-1-one;
ethyl 2-[(1,1-dioxo-3,4-dihydro-2H-thiochromen-6-yl)amino]-4-[[(1S)-2-hydroxy-1-phenyl-ethyl]amino]pyrimidine-5-carboxylate;
ethyl 4-[[(1S)-2-hydroxy-1-phenyl-ethyl]amino]-2-[(2-oxo-1,3,4,5-tetrahydro-1-benzazepin-8-yl)amino]pyrimidine-5-carboxylate;
6-[[4-[[(1S)-2-hydroxy-1-(3-methoxyphenyl)ethyl]amino]-5-(3-methyl-1,2,4-oxadiazol-5-yl)pyrimidin-2-yl]amino]-3,4-dihydro-2H-isoquinolin-1-one;
ethyl 2-[[5-ethoxycarbonyl-4-[[(1S)-2-hydroxy-1-phenyl-ethyl]amino]pyrimidin-2-yl]-(1-oxo-2,3,4,5-tetrahydro-2-benzazepin-7-yl)amino]-4-[[(1S)-2-hydroxy-1-phenyl-ethyl]amino]pyrimidine-5-carboxylate;
2-(3-fluoro-4-methylsulfonyl-anilino)-9-[(1S)-2-hydroxy-1-phenyl-ethyl]-7H-purin-8-one;
6-[[4-[[(1S)-2-hydroxy-1-phenyl-ethyl]amino]-5-(3-methyl-1,2,4-oxadiazol-5-yl)pyrimidin-2-yl]amino]-3,3-dimethyl-2,4-dihydroisoquinolin-1-one;
5-[[4-[[(1S)-2-hydroxy-1-phenyl-ethyl]amino]-5-(3-methyl-1,2,4-oxadiazol-5-yl)pyrimidin-2-yl]amino]-3,3-dimethyl-isoindolin-1-one;
6-[[4-[[(1S)-2-hydroxy-1-phenyl-ethyl]amino]-5-(3-vinyl-1,2,4-oxadiazol-5-yl)pyrimidin-2-yl]amino]-2-(2-trimethylsilylethoxymethyl)-3,4-dihydroisoquinolin-1-one;
[6-[[4-[[(1S)-2-hydroxy-1-phenyl-ethyl]amino]-5-(3-methyl-1,2,4-oxadiazol-5-yl)pyrimidin-2-yl]amino]-1-oxo-3,4-dihydroisoquinolin-2-yl]methyl 2,2-dimethylpropanoate;
6-[[4-[[(1S)-2-hydroxy-1-phenyl-ethyl]amino]-5-(3-methyl-1,2,4-oxadiazol-5-yl)pyrimidin-2-yl]amino]-2-(2-trimethylsilylethoxymethyl)-3,4-dihydroisoquinolin-1-one;
(2S)-2-[[5-[3-[(dimethylamino)methyl]-1,2,4-oxadiazol-5-yl]-2-[(1,1-dioxo-3,4-dihydro-2H-thiochromen-6-yl)amino]pyrimidin-4-yl]amino]-2-phenyl-ethanol;
6-[[4-[[(1S)-2-hydroxy-1-phenyl-ethyl]amino]-5-(1-methylpyrazol-4-yl)pyrimidin-2-yl]amino]-3,4-dihydro-2H-isoquinolin-1-one;
5-[[5-[3-[(dimethylamino)methyl]-1,2,4-oxadiazol-5-yl]-4-[[(1S)-2-hydroxy-1-phenyl-ethyl]amino]pyrimidin-2-yl]amino]isoindolin-1-one;
(2S)-2-[[2-[(1,1-dioxo-3,4-dihydro-2H-thiochromen-6-yl)amino]-5-[3-(pyrrolidin-1-ylmethyl)-1,2,4-oxadiazol-5-yl]pyrimidin-4-yl]amino]-2-phenyl-ethanol;
5-[[4-[[(1S)-2-hydroxy-1-phenyl-ethyl]amino]-5-[3-(pyrrolidin-1-ylmethyl)-1,2,4-oxadiazol-5-yl]pyrimidin-2-yl]amino]isoindolin-1-one

or a pharmaceutically acceptable salt, stereoisomer, solvate, or tautomer thereof.

In some embodiments, the compound is:
4-[[(1S)-2-hydroxy-1-phenyl-ethyl]amino]-N-methyl-2-[(2-methyl-1-oxo-3,4-dihydroisoquinolin-6-yl)amino]pyrimidine-5-carboxamide;
ethyl 4-[[(1S)-2-hydroxy-1-phenyl-ethyl]amino]-2-[(1-oxo-3,4-dihydro-2H-isoquinolin-6-yl)amino]pyrimidine-5-carboxylate;
ethyl 2-(3-fluoro-4-methylsulfonyl-anilino)-4-[[(1S)-2-hydroxy-1-phenyl-ethyl]amino]-pyrimidine-5-carboxylate;
2-(3-fluoro-4-methylsulfonyl-anilino)-4-[[(1S)-2-hydroxy-1-phenyl-ethyl]amino]-N-methyl-pyrimidine-5-carboxamide;
4-[[(1S)-2-hydroxy-1-phenyl-ethyl]amino]-N-methyl-2-(3-methyl-4-methylsulfonyl-anilino)pyrimidine-5-carboxamide;
ethyl 4-[[(1S)-2-hydroxy-1-phenyl-ethyl]amino]-2-[(2-methyl-1-oxo-3,4-dihydroisoquinolin-6-yl)amino]pyrimidine-5-carboxylate;
ethyl 4-[[(1S)-2-hydroxy-1-phenyl-ethyl]amino]-2-(3-methyl-4-methylsulfonyl-anilino)pyrimidine-5-carboxylate;
N-ethyl-4-[[(1S)-2-hydroxy-1-phenyl-ethyl]amino]-2-(3-methyl-4-methylsulfonyl-anilino)pyrimidine-5-carboxamide;
4-[[(1S)-2-hydroxy-1-phenyl-ethyl]amino]-2-(3-methyl-4-methylsulfonyl-anilino)-N-(2,2,2-trifluoroethyl)-pyrimidine-5-carboxamide;
N-ethyl-4-[[(1S)-2-hydroxy-1-phenyl-ethyl]amino]-2-[(2-methyl-1-oxo-3,4-dihydroisoquinolin-6-yl)amino]pyrimidine-5-carboxamide;
ethyl 4-[[(1S)-2-hydroxy-1-phenyl-ethyl]amino]-2-(3-methoxy-4-methylsulfonyl-anilino)pyrimidine-5-carboxylate;
ethyl 2-(4-ethylsulfonylanilino)-4-[[(1S)-2-hydroxy-1-phenyl-ethyl]amino]pyrimidine-5-carboxylate;
ethyl 4-[[(1S)-2-hydroxy-1-phenyl-ethyl]amino]-2-(4-methylsulfonylanilino)pyrimidine-5-carboxylate;
(2S)-2-[[2-(3-methyl-4-methylsulfonyl-anilino)-5-(3-methyl-1,2,4-oxadiazol-5-yl)pyrimidin-4-yl]amino]-2-phenyl-ethanol;
2-(3-fluoro-4-methylsulfonyl-anilino)-4-[[(1S)-2-hydroxy-1-phenyl-ethyl]amino]pyrimidine-5-carboxamide;
(2S)-2-[[2-(3-fluoro-4-methylsulfonyl-anilino)-5-(1,3,4-oxadizol-2-yl)pyrimidin-4-yl]amino]-2-phenyl-ethanol;
(2S)-2-[[2-(3-fluoro-4-methylsulfonyl-anilino)-5-(3-methyl-1,2,4-oxadiazol-5-yl)pyrimidin-4-yl]amino]-2-phenyl-ethanol;
2-hydroxyethyl 2-(3-fluoro-4-methylsulfonyl-anilino)-4-[[(1S)-2-hydroxy-1-phenyl-ethyl]amino]pyrimidine-5-carboxylate;
ethyl 2-[(2-ethyl-1-oxo-3,4-dihydroisoquinolin-6-yl)amino]-4-[[(1S)-2-hydroxy-1-phenyl-ethyl]amino]pyrimidine-5-carboxylate;
ethyl 2-[4-(dimethylcarbamoyl)anilino]-4-[[(1S)-2-hydroxy-1-phenyl-ethyl]amino]pyrimidine-5-carboxylate

or a pharmaceutically acceptable salt, stereoisomer, solvate, or tautomer thereof.

It should be understood that all isomeric forms are included within the present invention, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the invention, and pharmaceutically acceptable salts, hydrates, solvates, stereoisomers and prodrugs thereof may exist in their tautomeric form (for example, as an amide or imino ether). All such tautomeric forms are contemplated herein as part of the present invention.

The compounds of the invention may contain asymmetric or chiral centers, and, therefore, exist in different stereoisomeric forms. It is intended that all stereoisomeric forms of the compounds of the invention as well as mixtures thereof, including racemic mixtures, form part of the present invention. In addition, the present invention embraces all geometric and positional isomers. For example, if a compound of the invention incorporates a double bond or a fused ring, both the cis- and trans-forms, as well as mixtures, are embraced within the scope of the invention. each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the invention may be atropisomers (e.g., substituted biaryls) and are considered as part of this invention. Enantiomers can also be separated by use of a chiral HPLC column.

It is also possible that the compounds of the invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds of Formula I may form salts which are also within the scope of this invention. Reference to a compound of the Formula herein is understood to include reference to salts thereof, unless otherwise indicated.

The present invention relates to compounds which are modulators of hematopoietic progenitor kinase 1 (HPK1).

In one embodiment, the compounds of the present invention are inhibitors of hematopoietic progenitor kinase 1 (HPK1).

In some embodiments, the compounds of Formula I are selective inhibitors of hematopoietic progenitor kinase 1 (HPK1).

The present invention relates to compounds which are modulators of hematopoietic progenitor kinase 1 (HPK1).

In one embodiment, the compounds of the present invention are inhibitors of hematopoietic progenitor kinase 1 (HPK1).

In some embodiments, the compounds of Formula I are selective inhibitors of hematopoietic progenitor kinase 1 (HPK1).

The present invention relates to compounds which are modulators of leucine rich repeat kinase 2 (LRRK2) protein.

In one embodiment, the compounds of the present invention are inhibitors of leucine rich repeat kinase 2 (LRRK2) protein.

In some embodiments, the compounds of Formula I are selective inhibitors of leucine rich repeat kinase 2 (LRRK2) protein.

The present invention relates to compounds which are modulators of FMS-like tyrosine kinase 3 (FLT3) gene.

In one embodiment, the compounds of the present invention are inhibitors of FMS-like tyrosine kinase 3 (FLT3) gene.

In some embodiments, the compounds of Formula I are selective inhibitors of FMS-like tyrosine kinase 3 (FLT3) gene.

The present invention relates to compounds which are modulators of interleukin-1 receptor-associated kinase 1 (IRAK1).

In one embodiment, the compounds of the present invention are inhibitors of interleukin-1 receptor-associated kinase 1 (IRAK1).

In some embodiments, the compounds of Formula I are selective inhibitors of interleukin-1 receptor-associated kinase 1 (IRAK1).

The present invention relates to compounds which are modulators of interleukin-1 receptor-associated kinase 4 (IRAK4).

In one embodiment, the compounds of the present invention are inhibitors of interleukin-1 receptor-associated kinase 4 (IRAK4).

In some embodiments, the compounds of Formula I are selective inhibitors of interleukin-1 receptor-associated kinase 4 (IRAK4).

The present invention relates to compounds which are modulators of Janus kinases (JAKs), including Janus kinase 1 (JAK1), Janus kinase 2 (JAK2), Janus kinase 3 (JAK3), and tyrosine kinase 2 (TYK2).

In one embodiment, the compounds of the present invention are inhibitors of Janus kinases (JAKs), including Janus kinase 1 (JAK1), Janus kinase 2 (JAK2), Janus kinase 3 (JAK3), and tyrosine kinase 2 (TYK2).

In some embodiments, the compounds of Formula I are selective inhibitors of Janus kinases (JAKs), including Janus kinase 1 (JAK1), Janus kinase 2 (JAK2), Janus kinase 3 (JAK3), and tyrosine kinase 2 (TYK2).

The invention is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof.

### Method of Synthesizing the Compounds

The compounds of the present invention may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedure A which comprises different sequences of assembling intermediates or compounds. Starting materials are either commercially available or made by known procedures in the reported literature or as illustrated below.

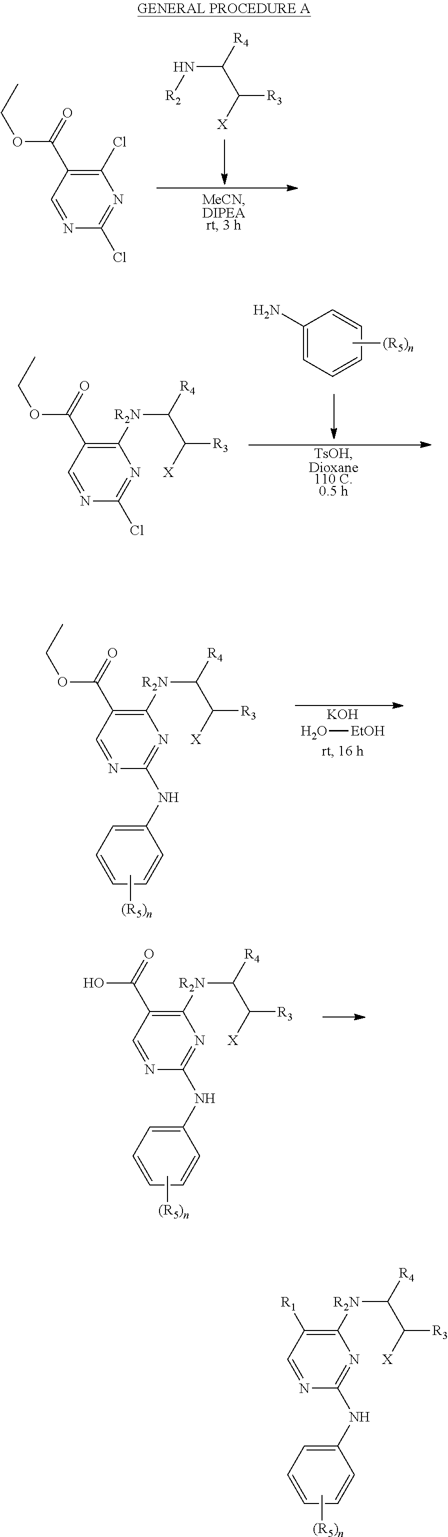

### Methods of Using the Disclosed Compounds

Another aspect of the invention relates to a method of treating a disease or disorder associated with modulation of hematopoietic progenitor kinase 1 (HPK1). The method comprises administering to a patient in need of a treatment for diseases or disorders associated with modulation of HPK1 an effective amount the compositions and compounds of Formula (I).

In another aspect, the present invention is directed to a method of inhibiting hematopoietic progenitor kinase 1 (HPK1). The method involves administering to a patient in need thereof an effective amount of a compound of Formula (I).

Another aspect of the present invention relates to a method of treating, preventing, inhibiting or eliminating a disease or disorder in a patient associated with the inhibition of hematopoietic progenitor kinase 1 (HPK1), the method comprising administering to a patient in need thereof an effective amount of a compound of Formula (I). In one embodiment, the disease may be, but not limited to, cancer.

The present invention also relates to the use of an inhibitor of hematopoietic progenitor kinase 1 (HPK1) for the preparation of a medicament used in the treatment, prevention, inhibition or elimination of a disease or condition mediated by HPK1, wherein the medicament comprises a compound of Formula (I).

In another aspect, the present invention relates to a method for the manufacture of a medicament for treating, preventing, inhibiting, or eliminating a disease or condition mediated by hematopoietic progenitor kinase 1 (HPK1), wherein the medicament comprises a compound of Formula (I).

Another aspect of the present invention relates to a compound of Formula (I) for use in the manufacture of a medicament for treating a disease associated with inhibiting hematopoietic progenitor kinase 1 (HPK1).

In another aspect, the present invention relates to the use of a compound of Formula (I) in the treatment of a disease associated with inhibiting hematopoietic progenitor kinase 1 (HPK1).

Another aspect of the invention relates to a method of treating a disease or disorder associated with modulation of leucine rich repeat kinase 2 (LRRK2) protein. The method comprises administering to a patient in need of a treatment for diseases or disorders associated with modulation of LRRK2 an effective amount the compositions and compounds of Formula (I).

In another aspect, the present invention is directed to a method of inhibiting leucine rich repeat kinase 2 (LRRK2) protein. The method involves administering to a patient in need thereof an effective amount of a compound of Formula (I).

Another aspect of the present invention relates to a method of treating, preventing, inhibiting or eliminating a disease or disorder in a patient associated with the inhibition of leucine rich repeat kinase 2 (LRRK2) protein, the method comprising administering to a patient in need thereof an effective amount of a compound of Formula (I).

The present invention also relates to the use of an inhibitor of leucine rich repeat kinase 2 (LRRK2) protein for the preparation of a medicament used in the treatment, prevention, inhibition or elimination of a disease or condition mediated by LRRK2, wherein the medicament comprises a compound of Formula (I).

In another aspect, the present invention relates to a method for the manufacture of a medicament for treating, preventing, inhibiting, or eliminating a disease or condition mediated by leucine rich repeat kinase 2 (LRRK2) protein, wherein the medicament comprises a compound of Formula (I).

Another aspect of the present invention relates to a compound of Formula (I) for use in the manufacture of a medicament for treating a disease associated with inhibiting leucine rich repeat kinase 2 (LRRK2) protein.

In another aspect, the present invention relates to the use of a compound of Formula (I) in the treatment of a disease associated with inhibiting leucine rich repeat kinase 2 (LRRK2) protein.

In some embodiments, the leucine rich repeat kinase 2 (LRRK2) protein is a mutant LRRK2 protein.

Another aspect of the invention relates to a method of treating a disease or disorder associated with modulation of FMS-like tyrosine kinase 3 (FLT3) gene. The method comprises administering to a patient in need of a treatment for diseases or disorders associated with modulation of FLT3 an effective amount the compositions and compounds of Formula (I).

In another aspect, the present invention is directed to a method of inhibiting FMS-like tyrosine kinase 3 (FLT3) gene. The method involves administering to a patient in need thereof an effective amount of a compound of Formula (I).

Another aspect of the present invention relates to a method of treating, preventing, inhibiting or eliminating a disease or disorder in a patient associated with the inhibition of FMS-like tyrosine kinase 3 (FLT3) gene, the method comprising administering to a patient in need thereof an effective amount of a compound of Formula (I).

The present invention also relates to the use of an inhibitor of FMS-like tyrosine kinase 3 (FLT3) gene for the preparation of a medicament used in the treatment, prevention, inhibition or elimination of a disease or condition mediated by FLT3, wherein the medicament comprises a compound of Formula (I).

In another aspect, the present invention relates to a method for the manufacture of a medicament for treating, preventing, inhibiting, or eliminating a disease or condition mediated by FMS-like tyrosine kinase 3 (FLT3) gene, wherein the medicament comprises a compound of Formula (I).

Another aspect of the present invention relates to a compound of Formula (I) for use in the manufacture of a medicament for treating a disease associated with inhibiting FMS-like tyrosine kinase 3 (FLT3) gene.

In another aspect, the present invention relates to the use of a compound of Formula (I) in the treatment of a disease associated with inhibiting FMS-like tyrosine kinase 3 (FLT3) gene.

In some embodiments, the FMS-like tyrosine kinase 3 (FLT3) gene is a mutant FLT3 gene.

Another aspect of the invention relates to a method of treating a disease or disorder associated with modulation of interleukin-1 receptor-associated kinase 1 (IRAK1). The method comprises administering to a patient in need of a treatment for diseases or disorders associated with modulation of IRAK1 an effective amount the compositions and compounds of Formula (I).

In another aspect, the present invention is directed to a method of inhibiting interleukin-1 receptor-associated kinase 1 (IRAK1). The method involves administering to a patient in need thereof an effective amount of a compound of Formula (I).

Another aspect of the present invention relates to a method of treating, preventing, inhibiting or eliminating a disease or disorder in a patient associated with the inhibition of interleukin-1 receptor-associated kinase 1 (IRAK1), the method comprising administering to a patient in need thereof an effective amount of a compound of Formula (I).

The present invention also relates to the use of an inhibitor of interleukin-1 receptor-associated kinase 1 (IRAK1) for the preparation of a medicament used in the treatment, prevention, inhibition or elimination of a disease or condition mediated by IRAK1, wherein the medicament comprises a compound of Formula (I).

In another aspect, the present invention relates to a method for the manufacture of a medicament for treating, preventing, inhibiting, or eliminating a disease or condition mediated by interleukin-1 receptor-associated kinase 1 (IRAK1), wherein the medicament comprises a compound of Formula (I).

Another aspect of the present invention relates to a compound of Formula (I) for use in the manufacture of a medicament for treating a disease associated with inhibiting interleukin-1 receptor-associated kinase 1 (IRAK1).

In another aspect, the present invention relates to the use of a compound of Formula (I) in the treatment of a disease associated with inhibiting interleukin-1 receptor-associated kinase 1 (IRAK1).

Another aspect of the invention relates to a method of treating a disease or disorder associated with modulation of interleukin-1 receptor-associated kinase 4 (IRAK4). The method comprises administering to a patient in need of a treatment for diseases or disorders associated with modulation of IRAK4 an effective amount the compositions and compounds of Formula (I).

In another aspect, the present invention is directed to a method of inhibiting interleukin-1 receptor-associated kinase 4 (IRAK4). The method involves administering to a patient in need thereof an effective amount of a compound of Formula (I).

Another aspect of the present invention relates to a method of treating, preventing, inhibiting or eliminating a disease or disorder in a patient associated with the inhibition of interleukin-1 receptor-associated kinase 4 (IRAK4), the method comprising administering to a patient in need thereof an effective amount of a compound of Formula (I).

The present invention also relates to the use of an inhibitor of interleukin-1 receptor-associated kinase 4 (IRAK4) for the preparation of a medicament used in the treatment, prevention, inhibition or elimination of a disease or condition mediated by IRAK4, wherein the medicament comprises a compound of Formula (I).

In another aspect, the present invention relates to a method for the manufacture of a medicament for treating, preventing, inhibiting, or eliminating a disease or condition mediated by interleukin-1 receptor-associated kinase 4 (IRAK4), wherein the medicament comprises a compound of Formula (I).

Another aspect of the present invention relates to a compound of Formula (I) for use in the manufacture of a medicament for treating a disease associated with inhibiting interleukin-1 receptor-associated kinase 4 (IRAK4).

In another aspect, the present invention relates to the use of a compound of Formula (I) in the treatment of a disease associated with inhibiting interleukin-1 receptor-associated kinase 4 (IRAK4).

Another aspect of the invention relates to a method of treating a disease or disorder associated with modulation of Janus kinases (JAKs), including Janus kinase 1 (JAK1), Janus kinase 2 (JAK2), Janus kinase 3 (JAK3), and tyrosine kinase 2 (TYK2). The method comprises administering to a patient in need of a treatment for diseases or disorders associated with modulation of JAKs an effective amount the compositions and compounds of Formula (I).

In another aspect, the present invention is directed to a method of inhibiting Janus kinases (JAKs), including Janus kinase 1 (JAK1), Janus kinase 2 (JAK2), Janus kinase 3 (JAK3), and tyrosine kinase 2 (TYK2). The method involves administering to a patient in need thereof an effective amount of a compound of Formula (I).

Another aspect of the present invention relates to a method of treating, preventing, inhibiting or eliminating a disease or disorder in a patient associated with the inhibition of Janus kinases (JAKs), including Janus kinase 1 (JAK1), Janus kinase 2 (JAK2), Janus kinase 3 (JAK3), and tyrosine kinase 2 (TYK2), the method comprising administering to a patient in need thereof an effective amount of a compound of Formula (I).

The present invention also relates to the use of an inhibitor of Janus kinases (JAKs), including Janus kinase 1 (JAK1), Janus kinase 2 (JAK2), Janus kinase 3 (JAK3), and tyrosine kinase 2 (TYK2) for the preparation of a medicament used in the treatment, prevention, inhibition or elimination of a disease or condition mediated by JAKs, wherein the medicament comprises a compound of Formula (I).

In another aspect, the present invention relates to a method for the manufacture of a medicament for treating, preventing, inhibiting, or eliminating a disease or condition mediated by Janus kinases (JAKs), including Janus kinase 1 (JAK1), Janus kinase 2 (JAK2), Janus kinase 3 (JAK3), and tyrosine kinase 2 (TYK2), wherein the medicament comprises a compound of Formula (I).

Another aspect of the present invention relates to a compound of Formula (I) for use in the manufacture of a medicament for treating a disease associated with inhibiting Janus kinases (JAKs), including Janus kinase 1 (JAK1), Janus kinase 2 (JAK2), Janus kinase 3 (JAK3), and tyrosine kinase 2 (TYK2).

In another aspect, the present invention relates to the use of a compound of Formula (I) in the treatment of a disease associated with inhibiting Janus kinases (JAKs), including Janus kinase 1 (JAK1), Janus kinase 2 (JAK2), Janus kinase 3 (JAK3), and tyrosine kinase 2 (TYK2).

In some embodiments, the Janus kinase (JAK) is Janus kinase 1 (JAK1).

In some embodiments, the Janus kinase (JAK) is Janus kinase 2 (JAK2).

In some embodiments, the Janus kinase (JAK) is Janus kinase 3 (JAK3).

In some embodiments, the Janus kinase (JAK) is tyrosine kinase 2 (TYK2).

Another aspect of the invention relates to a method of treating cancer. The method comprises administering to a patient in need thereof an effective amount of a compound of Formula (I).

Another aspect of the invention relates to a method of treating or preventing cancer. The method comprises administering to a patient in need thereof an effective amount of a compound of Formula (I).

In one embodiment, the present invention relates to the use of an inhibitor of hematopoietic progenitor kinase 1 (HPK1) for the preparation of a medicament used in treatment, prevention, inhibition or elimination of a disease or disorder associated with cancer.

In some embodiments, the disease, disorder, or condition is selected from cancer, an autoimmune disease, an inflammatory disease, a viral infection, male fertility control, a benign hyperplasia, sepsis, a vascular disorder, an atherosclerotic disease, and a neurodegenerative disorder.

In some embodiments, the disease, disorder, or condition is cancer.

In some embodiments, the cancer is selected from bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, marginal zone lymphoma (MZL), follicular lymphoma (FL), diffuse large B-cell lymphoma (DLBCL), chronic lymphocytic leukemia/small lymphocytic lymphoma (CLL/SLL), acute myeloid leukemia (AML), and acute promyelocytic leukemia (APL).

In some embodiments, the disease, disorder, or condition is an autoimmune disease.

In some embodiments, the autoimmune disease is selected from chronic obstructive pulmonary disease (COPD), asthma, bronchitis, lupus, dermatomyositis, Sjogren's syndrome, multiple sclerosis, psoriasis, dry eye disease, type I diabetes mellitus and complications associated therewith, atopic eczema (atopic dermatitis), thyroiditis (Hashimoto's and autoimmune thyroiditis), contact dermatitis and further eczematous dermatitis, inflammatory bowel disease, interferonopathy, atherosclerosis, and amyotrophic lateral sclerosis.

In some embodiments, the asthma is selected from chronic asthma, inveterate asthma, intrinsic asthma, extrinsic asthma, dust asthma, and infantile asthma.

In some embodiments, the inveterate asthma is selected late asthma and airway hyperresponsiveness.

In some embodiments, the bronchitis is bronchial asthma.

In some embodiments, the lupus is selected from systemic lupus erythematosus (SLE), cutaneous lupus erythrematosis, and lupus nephritis.

In some embodiments, the inflammatory bowel disease is selected from Crohn's disease and ulcerative colitis.

In some embodiments, the disease, disorder, or condition is an inflammatory disease.

In some embodiments, the inflammatory disease is selected from rheumatoid arthritis, osteoarthritis, juvenile idiopathic arthritis, psoriasis, psoriatic arthritis, ankylosing spondylitis, allergic airway disease, chronic obstructive pulmonary disease (COPD), inflammatory liver disease, inflammatory bowel disease, endotoxin-driven disease state, and related diseases involving cartilage, such as that of the joints.

In some embodiments, the allergic airway disease is selected from asthma and rhinitis.

In some embodiments, the inflammatory liver disease is selected from primary biliary cholangitis (PBC) and primary sclerosing cholangitis (PSC).

In some embodiments, the inflammatory bowel disease is selected from Crohn's disease and ulcerative colitis.

In some embodiments, the disease, disorder, or condition is a viral infection.

In some embodiments, the viral infection is an infection by a virus selected from human adenovirus, human cytomegalovirus, Kaposi's sarcoma-associated herpesvirus, hepatitis A virus (HAV), hepatitis B virus (HBV), hepatitis C virus (HCV), Epstein-Barr virus, human immunodeficiency virus (HIV), HPS-associated hantaviruses, Sin Nombre virus, rotavirus, echovirus, foot-and-mouth disease virus, coxsackievirus, West Nile virus, Ebola virus, Ross River virus, human papillomavirus, and coronavirus.

In some embodiments, the viral infection is an infection by hepatitis B virus (HBV).

In some embodiments, the viral infection is an infection by human immunodeficiency virus (HIV).

In some embodiments, the disease, disorder, or condition is male fertility control.

In some embodiments, the disease, disorder, or condition is a benign hyperplasia.

In some embodiments, the benign hyperplasia is selected from benign hyperplasia of the prostate gland and benign hyperplasia of the mammary gland.

In some embodiments, the disease, disorder, or condition is sepsis.

In some embodiments, the disease, disorder, or condition is a vascular disorder.

In some embodiments, the vascular disorder is selected from erythromelalgia, peripheral artery disease, renal artery stenosis, Buerger's disease, Raynaud's disease, disseminated intravascular coagulation, and cerebrovascular disease.

In some embodiments, the disease, disorder, or condition is an atherosclerotic disorder.

In some embodiments, the atherosclerotic disease is selected from myocardial infarction and stroke.

In some embodiments, the disease, disorder, or condition is a neurodegenerative disorder.

In some embodiments, the neurodegenerative disorder is selected from Alzheimer's disease, vascular disease dementia, frontotemporal dementia (FTD), corticobasal degeneration (CBD), progressive supranuclear palsy (PSP), Lewy body dementia, tangle-predominant senile dementia, Pick's disease (PiD), argyrophilic grain disease, amyotrophic lateral sclerosis (ALS), other motor neuron diseases, Guam parkinsonism-dementia complex, FTDP-17, Lytico-Bodig disease, multiple sclerosis, traumatic brain injury (TBI), and Parkinson's disease.

Another aspect of the invention is directed to pharmaceutical compositions comprising a compound of Formula (I) and a pharmaceutically acceptable carrier. The pharmaceutical acceptable carrier may further include an excipient, diluent, or surfactant.

The disclosed compounds of the invention can be administered in effective amounts to treat or prevent a disorder and/or prevent the development thereof in subjects.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a Compound of the Invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate. Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention is directed to pharmaceutical compositions comprising a compound of Formula (I) and a pharmaceutically acceptable carrier. The pharmaceutical acceptable carrier may further include an excipient, diluent, or surfactant. In some embodiments, the pharmaceutical composition can further comprise an additional pharmaceutically active agent. In some embodiments, the additional therapeutic agent is selected from an immune checkpoint inhibitor, a cell-based therapy, and a cytokine therapy.

In some embodiments, the immune checkpoint antibody is selected from a PD-1 antibody, a PD-L1 antibody, a PD-L2 antibody, a CTLA-4 antibody, a TIM3 antibody, a LAG3 antibody, and a TIGIT antibody.

In some embodiments, the immune checkpoint inhibitor is an anti-PD-1 antibody.

In some embodiments, the immune checkpoint inhibitor is an anti-PD-L1 antibody.

In some embodiments, the cell-based therapy is a cancer vaccine.

In some embodiments, the cancer vaccine is selected from an anti-tumor vaccine or a vaccine based on neoantigens.

Cell-based therapies usually involve the removal of immune cells from a subject suffering from cancer, either from the blood or from a tumor. Immune cells specific for the tumor will be activated, grown, and returned to a subject suffering from cancer where the immune cells provide an immune response against the cancer.

In some embodiments, the immune cells are selected from natural killer cells, lymphokine-activated killer cells, cytotoxic T-cells, and dendritic cells.

In some embodiments, the cancer vaccine is natural killer cell-based.

In some embodiments, the cancer vaccine is lymphokine-activated killer cell-based.

In some embodiments, the cancer vaccine is cytotoxic T-cell-based.

In some embodiments, the cancer vaccine is dendritic cell-based.

In some embodiments, the cell-based therapy is selected from CAR-T therapy (e.g., chimeric antigen receptor T-cells which are T-cells engineered to target specific antigens), TIL therapy (e.g., administration of tumor-infiltrating lymphocytes), and TCR gene therapy.

In some embodiments, the cytokine therapy is interleukin-2 therapy.

In some embodiments, the cytokine therapy is interferon-alpha therapy.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Abbreviations

AcOH acetic acid
br Broad
aq. Aqueous
BSA bovine serum albumin
CC column chromatography (e.g. silica CC)
DCM Dichloromethane
DIPEA N,N-diisopropylethylamine
DMF N,N-dimethyl formamide
DMSO dimethyl sulfoxide
DTT Dithiothreitol
EDC, EDCI 1-(3-Dimethylaminopropyl)-3-ethylcarbodiimide
Et₂O diethyl ether
EtOAc ethyl acetate
EtOH Ethanol
FBS fetal bovine serum
h hour(s)
HOBt 1-hydroxybenzotriazole
HATU [bis(dimethylamino)methylene]-1H-1,2,3-triazolo[4,5-b]pyridinium 3-oxide hexafluorophosphate
HPLC high pressure (or performance) liquid chromatography
m Multiplet
M Molar
MeCN Acetonitrile
MeOH Methanol
MHz Megahertz
min Minutes
MS molecular sieves or mass spectroscopy
NBS N-bromosuccinimide
NMR nuclear magnetic resonance
PEG polyethylene glycol
rt room temperature
TFA trifluoroacetic acid
TFAA trifluoroacetic anhydride
TsOH toluenesulfonic acid (e.g. p-TsOH)

Purity and identity of all synthesized compounds were confirmed by LC-MS analysis performed on Shimadzu Analytical 10Avp equipped with PE SCIEX API 165 mass-, Sedex 75 ELSD-, and Shimadzu UV- (254 and 215) detectors. Separation was achieved with C18 column 100×4.6 mm, 5.0 μm, pore size 100 Å, water-acetonitrile+0.1 TFA, gradient 5 to 87 for 10 min.

Preparative HPLC purification was carried out on Shimadzu instrument equipped with SPD-10Avp detector and FRC-10A fraction collector. Separation was achieved with a column YMC-Pack ODS-AQ 250×20 mml, S-10 μm, 12 nm, gradient solution A-solution B (A: 1000 mL H₂O-226 μL TFA; B: 1000 mL CH₃CN-226 μL TFA).

In the Table 1 presented examples of the compound of Formula (I) that were synthesized in the frame of this invention, results of MS analysis and ID numbers for each compound for the further reference.

TABLE 1
Examples of the compounds and analytical data

| | | | MS | MS |
|---|---|---|---|---|
| Cmpd | | | calc. | found |
| # | Structure | IUPAC name | [MH⁺] | [MH⁺] |
| 1 | 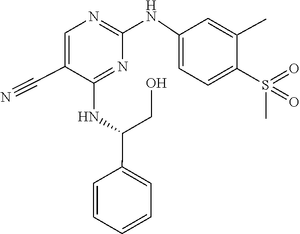 [SMILES: Cc1cc(Nc2ncc(C#N)c(N[C@H](CO)c3ccccc3)n2)ccc1S(C)(=O)=O] | 4-[[(1S)-2-hydroxy-1-phenyl- ethyl]amino]-2-(3-methyl-4- methylsulfonyl-anilino)pyrimidine-5- carbonitrile | 424.1 | 424 |
| 2 | 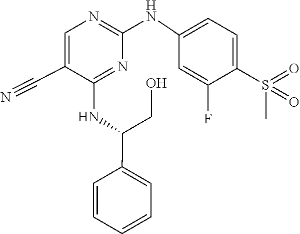 [SMILES: CS(=O)(=O)c1ccc(Nc2ncc(C#N)c(N[C@H](CO)c3ccccc3)n2)cc1F] | 2-(3-fluoro-4-methylsulfonyl- anilino)-4-[[(1S)-2-hydroxy-1-phenyl- ethyl]amino]pyrimidine-5-carbonitrile | 428.1 | 428 |
| 3 | 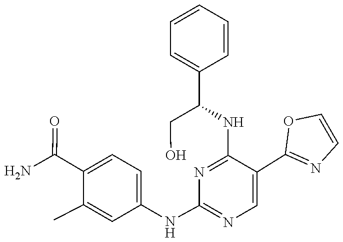 [SMILES: Cc1cc(Nc2ncc(-c3ncco3)c(N[C@H](CO)c3ccccc3)n2)ccc1C(N)=O] | 4-[[4-[[(1S)-2-hydroxy-1-phenyl- ethyl]amino]-5-oxazol-2-yl- pyrimidin-2-yl]amino]-2-methyl- benzamide | 431.1 | 431 |
| 4 | 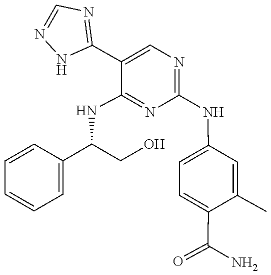 [SMILES: Cc1cc(Nc2ncc(-c3ncn[nH]3)c(N[C@H](CO)c3ccccc3)n2)ccc1C(N)=O] | 4-[[4-[[(1S)-2-hydroxy-1-phenyl- ethyl]amino]-5-(1H-1,2,4-triazol-5- yl)pyrimidin-2-yl]amino]-2-methyl- benzamide | 431.2 | 431 |
| 5 | 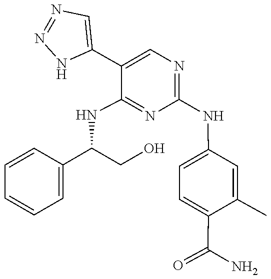 [SMILES: Cc1cc(Nc2ncc(-c3cnn[nH]3)c(N[C@H](CO)c3ccccc3)n2)ccc1C(N)=O] | 4-[[4-[[(1S)-2-hydroxy-1-phenyl- ethyl]amino]-5-(1H-triazol-5- yl)pyrimidin-2-yl]amino]-2-methyl- benzamide | 431.2 | 431 |
| 6 | 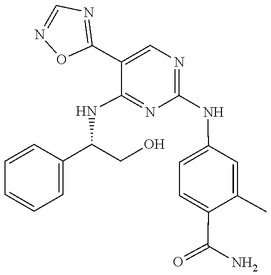 [SMILES: Cc1cc(Nc2ncc(-c3ncno3)c(N[C@H](CO)c3ccccc3)n2)ccc1C(N)=O] | 4-[[4-[[(1S)-2-hydroxy-1-phenyl- ethyl]amino]-5-(1,2,4-oxadiazol-5- yl)pyrimidin-2-yl]amino]-2-methyl- benzamide | 432.2 | 432 |
| 7 | 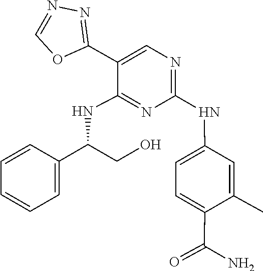 [SMILES: Cc1cc(Nc2ncc(-c3nnco3)c(N[C@H](CO)c3ccccc3)n2)ccc1C(N)=O] | 4-[[4-[[(1S)-2-hydroxy-1-phenyl- ethyl]amino]-5-1,3,4-oxadiazol-2- yl)pyrimidin-2-yl]amino]-2-methyl- benzamide | 432.2 | 432 |
| 8 | 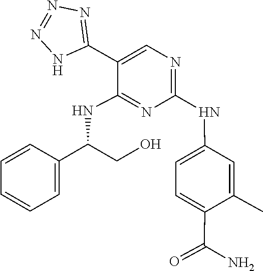 [SMILES: Cc1cc(Nc2ncc(-c3nnn[nH]3)c(N[C@H](CO)c3ccccc3)n2)ccc1C(N)=O] | 4-[[4-[[(1S)-2-hydroxy-1-phenyl- ethyl]amino]-5-(1H-tetrazol-5- yl)pyrimidin-2-yl]amino]-2-methyl- benzamide | 432.2 | 432 |
| 9 | 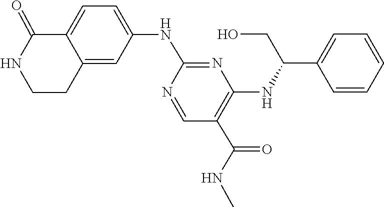 [SMILES: CNC(=O)c1cnc(Nc2ccc3c(c2)CCNC3=O)nc1N[C@H](CO)c1ccccc1] | 4-[[(1S)-2-hydroxy-1-phenyl- ethyl]amino]-N-methyl-2-[(1-oxo-3,4- dihydro-2H-isoquinolin-6- yl)amino]pyrimidine-5-carboxamide | 433.2 | 433 |
| 10 | 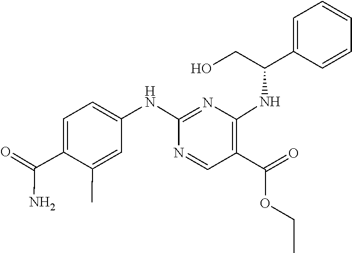 [SMILES: CCOC(=O)c1cnc(Nc2ccc(C(N)=O)c(C)c2)nc1N[C@H](CO)c1ccccc1] | ethyl 2-(4-carbamoyl)-3-methyl- anilino)-4-[[(1S)-2-hydroxy-1-phenyl- ethyl]amino]pyrimidine-5-carboxylate | 436.2 | 436 |
| 11 | 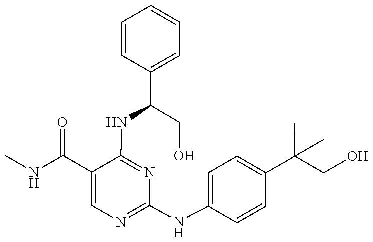 [SMILES: CNC(=O)c1cnc(Nc2ccc(C(C)(C)CO)cc2)nc1N[C@H](CO)c1ccccc1] | 2-[4-(2-hydroxy-1,1-dimethyl- ethyl)anilino]-4-[[(1S)-2-hydroxy-2- phenyl-ethyl]amino]-N-methyl- pyrimidine-5-carboxamide | 436.2 | 436 |
| 12 | 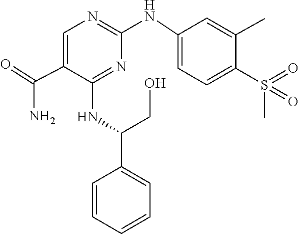 [SMILES: Cc1cc(Nc2ncc(C(N)=O)c(N[C@H](CO)c3ccccc3)n2)ccc1S(C)(=O)=O] | 4-[[(1S)-2-hydroxy-1-phenyl- ethyl]amino]-2-(3-methyl-4- methylsulfonyl-anilino)pyrimidine-5- carboxamide | 442.2 | 442 |
| 13 | 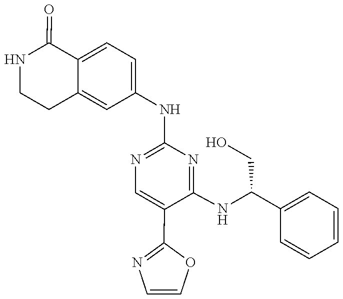 [SMILES: O=C1NCCc2cc(Nc3ncc(-c4ncco4)c(N[C@H](CO)c4ccccc4)n3)ccc21] | 6-[[4-[[(1S)-2-hydroxy-1-phenyl- ethyl]amino]-5-oxazol-2-yl- pyrimidin-2-yl]amino]-3,4-dihydro- 2H-isoquinolin-1-one | 443.2 | 443 |
| 14 | 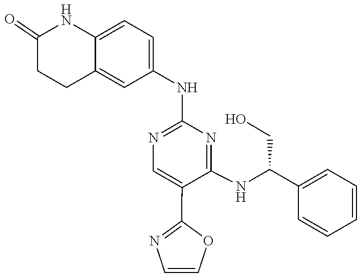 [SMILES: O=C1CCc2cc(Nc3ncc(-c4ncco4)c(N[C@H](CO)c4ccccc4)n3)ccc2N1] | 6-[[4-[[(1S)-2-hydroxy-1-phenyl- ethyl]amino]-5-oxazol-2-yl- pyrimidin-2-yl]amino]-3,4-dihydro- 1H-quinolin-2-one | 443.2 | 443 |
| 15 | 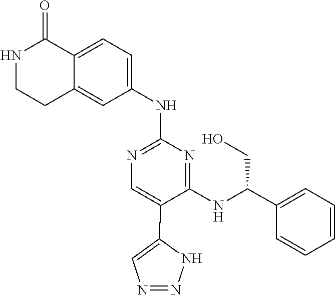 [SMILES: O=C1NCCc2cc(Nc3ncc(-c4cnn[nH]4)c(N[C@H](CO)c4ccccc4)n3)ccc21] | 6-[[4-[[(1S)-2-hydroxy-1-phenyl- ethyl]amino]-5-(1H-triazol-5- yl)pyrimidin-2-yl]amino]-3,4- dihydro-2H-isoquinolin-1-one | 443.2 | 443 |
| 16 | 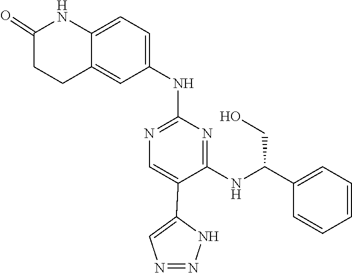 [SMILES: O=C1CCc2cc(Nc3ncc(-c4cnn[nH]4)c(N[C@H](CO)c4ccccc4)n3)ccc2N1] | 6-[[4-[[(1S)-2-hydroxy-1-phenyl- ethyl]amino]-5-(1H-triazol-5- yl)pyrimidin-2-yl]amino]-3,4- dihydro-1H-quinolin-2-one | 443.2 | 443 |
| 17 | 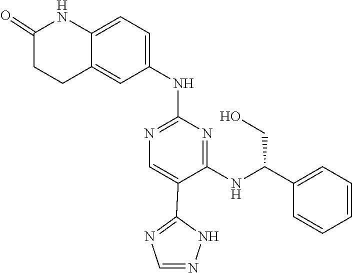 [SMILES: O=C1CCc2cc(Nc3ncc(-c4ncn[nH]4)c(N[C@H](CO)c4ccccc4)n3)ccc2N1] | 6-[[4-[[(1S)-2-hydroxy-1-phenyl- ethyl]amino]-5-(1H-1,2,4-triazol-5- yl)pyrimidin-2-yl]amino]-3,4- dihydro-1H-quinolin-2-one | 443.2 | 443 |
| 18 | 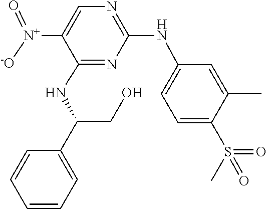 | (2S)-2-[[2-(3-methyl-4- methylsulfonyl-anilino)-5-nitro- pyrimidin-4-yl]amino]-2-phenyl- ethanol | 444.1 | 444 |
| 19 | 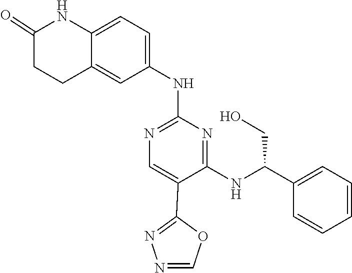 [SMILES: O=C1CCc2cc(Nc3ncc(-c4nnco4)c(N[C@H](CO)c4ccccc4)n3)ccc2N1] | 6-[[4-[[(1S)-2-hydroxy-1-phenyl- ethyl]amino]-5-(1,3,4-oxadiazol-2- yl)pyrimidin-2-yl]amino]-3,4- dihydro-1H-quinolin-2-one | 444.2 | 444 |
| 20 | 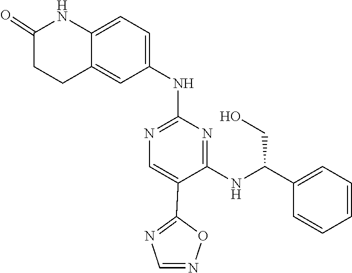 [SMILES: O=C1CCc2cc(Nc3ncc(-c4ncno4)c(N[C@H](CO)c4ccccc4)n3)ccc2N1] | 6-[[4-[[(1S)-2-hydroxy-1-phenyl- ethyl]amino]-5-(1,2,4-oxadiazol-5- yl)pyrimidin-2-yl]amino]-3,4- dihydro-1H-quinolin-2-one | 444.2 | 444 |
| 21 | 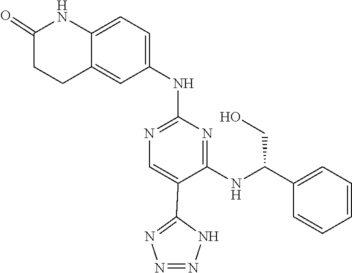 [SMILES: O=C1CCc2cc(Nc3ncc(-c4nnn[nH]4)c(N[C@H](CO)c4ccccc4)n3)ccc2N1] | 6-[[4-[[(1S)-2-hydroxy-1-phenyl- ethyl]amino]-5-(1H-tetrazol-5- y)pyrimidin-2-yl]amino]-3,4- dihydro-1H-quinolin-2-one | 444.2 | n/d |
| 22 | 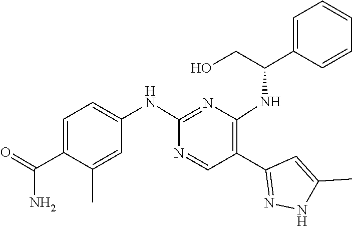 [SMILES: Cc1cc(-c2cnc(Nc3ccc(C(N)=O)c(C)c3)nc2N[C@H](CO)c2ccccc2)n[nH]1] | 4-[[4-[[(1S)-2-hydroxy-1-phenyl- ethyl]amino]-5-(5-methyl-1H- pyrazol-3-yl)pyrimidin-2-yl]amino]- 2-methyl-benzamide | 444.2 | 444 |
| 23 | 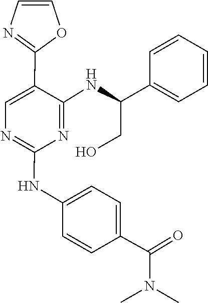 [SMILES: CN(C)C(=O)c1ccc(Nc2ncc(-c3ncco3)c(N[C@H](CO)c3ccccc3)n2)cc1] | 4-[[4-[[(1S)-2-hydroxy-1-phenyl- ethyl]amino]-5-oxazol-2-yl- pyrimidin-2-yl]amino]-N,N-dimethyl- benzamide | 445.2 | 445 |
| 24 | 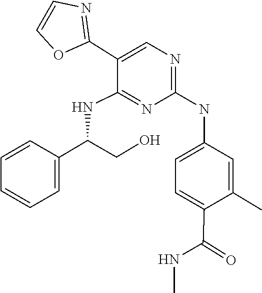 | 4-[[4-[[(1S)-2-hydroxy-1-phenyl- ethyl]amino]-5-oxazol-2-yl- pyrimidin-2-yl]amino]-N,2-dimethyl- benzamide | 445.2 | 445 |
| 25 | 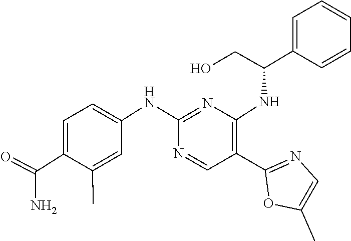 [SMILES: Cc1cnc(-c2cnc(Nc3ccc(C(N)=O)c(C)c3)nc2N[C@H](CO)c2ccccc2)o1] | 4-[[4-[[(1S)-2-hydroxy-1-phenyl- ethyl]amino]-5-(5-methyloxazol-2- yl)pyrimidin-2-yl]amino]-2-methyl- benzamide | 445.2 | 445 |
| 26 | 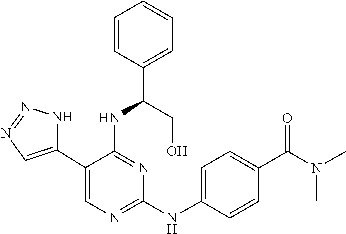 [SMILES: CN(C)C(=O)c1ccc(Nc2ncc(-c3cnn[nH]3)c(N[C@H](CO)c3ccccc3)n2)cc1] | 4-[[4-[[(1S)-2-hydroxy-1-phenyl- ethyl]amino]-5-(1H-triazol-5- yl)pyrimidin-2-yl]amino]-N,N- dimethyl-benzamide | 445.2 | 445 |
| 27 | 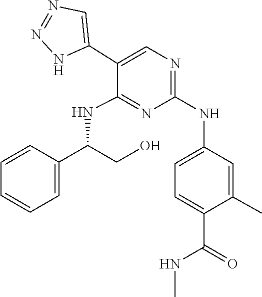 [SMILES: CNC(=O)c1ccc(Nc2ncc(-c3cnn[nH]3)c(N[C@H](CO)c3ccccc3)n2)cc1C] | 4-[[4-[[(1S)-2-hydroxy-1-phenyl- ethyl]amino]-5-(1H-triazol-5- yl)pyrimidin-2-yl]amino]-N,2- dimethyl-benzamide | 445.2 | 445 |
| 28 | 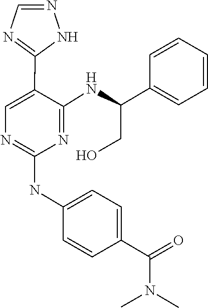 | 4-[[4-[[(1S)-2-hydroxy-1-phenyl- ethyl]amino]-5-(1H-1,2,4-triazol-5- yl)pyrimidin-2-yl]amino]-N,N- dimethyl-benzamide | 445.2 | 445 |
| 29 | 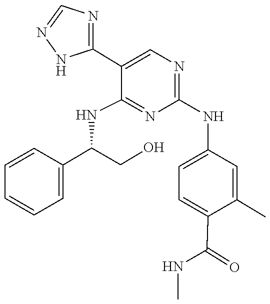 [SMILES: CNC(=O)c1ccc(Nc2ncc(-c3ncn[nH]3)c(N[C@H](CO)c3ccccc3)n2)cc1C] | 4-[[4-[[(1S)-2-hydroxy-1-phenyl- ethyl]amino]-5-(1H-1,2,4-triazol-5- yl)pyrimidin-2-yl]amino]-N,2- dimethyl-benzamide | 445.2 | 445 |
| 30 | 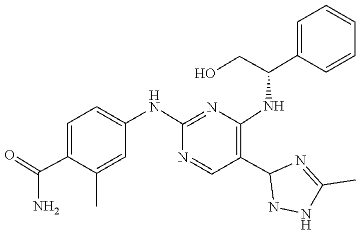 | 4-[[4-[[(1S)-2-hydroxy-1-phenyl- ethyl]amino]-5-(5-methyl-1H-1,2,4- triazol-3-yl)pyrimidin-2-yl]amino]-2- methyl-benzamide | 445.2 | 445 |
| 31 | 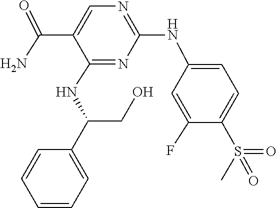 [SMILES: CS(=O)(=O)c1ccc(Nc2ncc(C(N)=O)c(N[C@H](CO)c3ccccc3)n2)cc1F] | 2-(3-fluoro-4-methylsulfonyl- anilino)-4-[[(1S)-2-hydroxy-1-phenyl- ethyl]amino]pyrimidine-5- carboxamide | 446.1 | 446 |
| 32 | 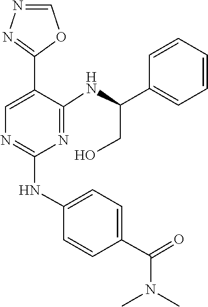 [SMILES: CN(C)C(=O)c1ccc(Nc2ncc(-c3nnco3)c(N[C@H](CO)c3ccccc3)n2)cc1] | 4-[[4-[[(1S)-2-hydroxy-1-phenyl- ethyl]amino]-5-(1,3,4-oxadiazol-2- yl)pyrimidin-2-yl]amino]-N,N- dimethyl-benzamide | 446.2 | 446 |
| 33 | 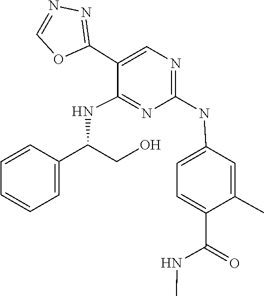 | 4 -[[4-[[(1S)-2-hydroxy-1-phenyl- ethyl]amino]-5-(1,3,4-oxadiazol-2- yl)pyrimidin-2-yl]amino]-N,2- dimethyl-benzamide | 446.2 | 446 |
| 34 | 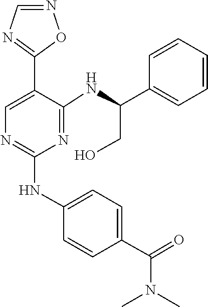 [SMILES: CN(C)C(=O)c1ccc(Nc2ncc(-c3ncno3)c(N[C@H](CO)c3ccccc3)n2)cc1] | 4-[[4-[[(1S)-2-hydroxy-1-phenyl- ethyl]amino]-5-(1,2,4-oxadiazol-5- yl)pyrimidin-2-yl]amino]-N,N- dimethyl-benzamide | 446.2 | 446 |
| 35 | 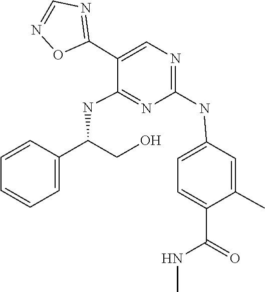 | 4-[[4-[[(1S)-2-hydroxy-1-phenyl- ethyl]amino]-5-(1,2,4-oxadiazol-5- yl)pyrimidin-2-yl]amino]-N,2- dimethyl-benzamide | 446.2 | 446 |
| 36 | 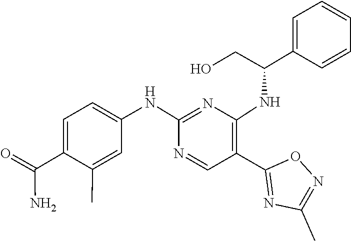 [SMILES: Cc1noc(-c2cnc(Nc3ccc(C(N)=O)c(C)c3)nc2N[C@H](CO)c2ccccc2)n1] | 4-[[4-[[(1S)-2-hydroxy-1-phenyl- ethyl]amino]-5-(3-mehtyl-1,2,4- oxadiazol-5-yl)pyrimidin-2- yl]amino]-2-methyl-benzamide | 446.2 | 446 |
| 37 | 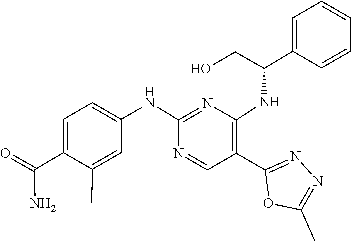 [SMILES: Cc1nnc(-c2cnc(Nc3ccc(C(N)=O)c(C)c3)nc2N[C@H](CO)c2ccccc2)o1] | 4-[[4-[[(1S)-2-hydroxy-1-phenyl- ethyl]amino]-5-(5-methyl-1,3,4- oxadiazol-2-yl)pyrimidin-2- yl]amino]-2-methyl-benzamide | 446.2 | 446 |
| 38 | 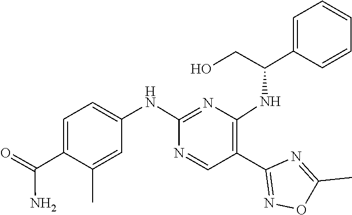 [SMILES: Cc1nc(-c2cnc(Nc3ccc(C(N)=O)c(C)c3)nc2N[C@H](CO)c2ccccc2)no1] | 4-[[4-[[(1S)-2-hydroxy-1-phenyl- ethyl]amino]-5-(5-methyl-1,2,4- oxadiazol-3-yl)pyrimidin-2- yl]amino]-2-methyl-benzamide | 446.2 | 446 |
| 39 | 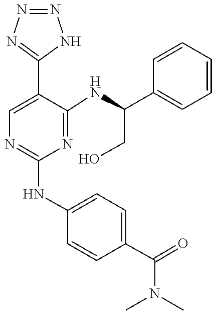 [SMILES: CN(C)C(=O)c1ccc(Nc2ncc(-c3nnn[nH]3)c(N[C@H](CO)c3ccccc3)n2)cc1] | 4-[[4-[[(1S)-2-hydroxy-1-phenyl- ethyl]amino]-5-(1H-tetrazol-5- yl)pyrimidin-2-yl]amino]-N,N- dimethyl-benzamide | 446.2 | 446 |
| 40 | 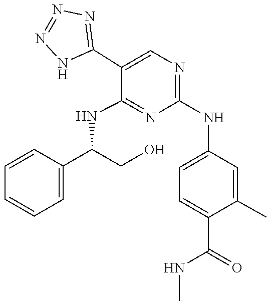 [SMILES: CNC(=O)c1ccc(Nc2ncc(-c3nnn[nH]3)c(N[C@H](CO)c3ccccc3)n2)cc1C] | 4 -[[4-[[(1S)-2-hydroxy-1-phenyl- ethyl]amino]-5-(1H-tetrazol-5- yl)pyrimidin-2-yl]amino]-N,2- dimethyl-benzamide | 446.2 | 446 |
| 41 | 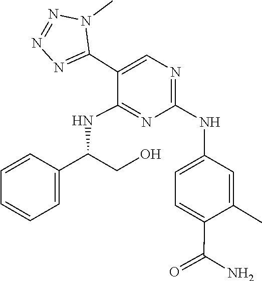 [SMILES: Cc1cc(Nc2ncc(-c3nnnn3C)c(N[C@H](CO)c3ccccc3)n2)ccc1C(N)=O] | 4-[[4-[[(1S)-2-hydroxy-1-phenyl- ethyl]amino]-5-(1-methyltetrazol-5- yl)pyrimidin-2-yl]amino]-2-methyl- benzamide | 446.2 | 446 |
| 42 | 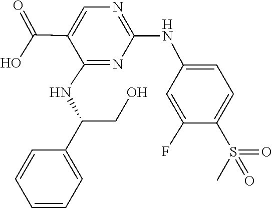 [SMILES: CS(=O)(=O)c1ccc(Nc2ncc(C(=O)O)c(N[C@H](CO)c3ccccc3)n2)cc1F] | 2-(3-fluoro-4-methylsulfonyl- anilino)-4-[[(1S)-2-hydroxy-1-phenyl- ethyl]amino]pyrimidine-5-carboxylic acid | 447.1 | 447 |
| 43 | 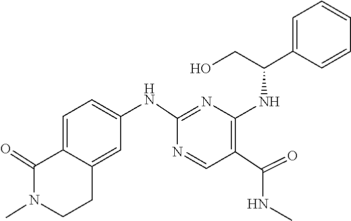 [SMILES: CNC(=O)c1cnc(Nc2ccc3c(c2)CCN(C)C3=O)nc1N[C@H](CO)c1ccccc1] | 4-[[(1S)-2-hydroxy-1-phenyl- ethyl]amino]-N-methyl-2-[(2-methyl- 1-oxo-3,4-dihydroisoquinolin-6- yl)amino]pyrimidine-5-carboxamide | 447.2 | 447 |
| 44 | 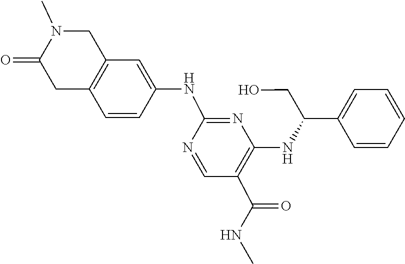 [SMILES: CNC(=O)c1cnc(Nc2ccc3c(c2)CN(C)C(=O)C3)nc1N[C@H](CO)c1ccccc1] | 4-[[(1S)-2-hydroxy-1-phenyl- ethyl]amino]-N-methyl-2-[(2-methyl- 3-oxo-1,4-dihydroisoquinolin-7- yl)amino]pyrimidine-5-carboxamide | 447.2 | 447 |
| 45 | 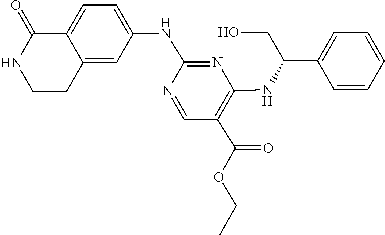 [SMILES: CCOC(=O)c1cnc(Nc2ccc3c(c2)CCNC3=O)nc1N[C@H](CO)c1ccccc1] | ethyl 4-[[(1S)-2-hydroxy-1-phenyl- ethyl]amino]-2-[(1-oxo-3,4-dihydro- 2H-isoquinolin-6- yl)amino]pyrimidine-5-carboxylate | 448.2 | 448 |
| 46 | 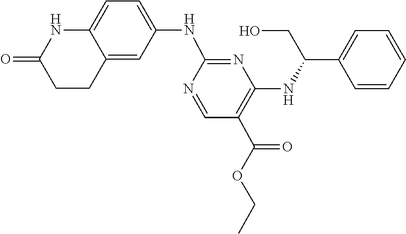 [SMILES: CCOC(=O)c1cnc(Nc2ccc3c(c2)CCC(=O)N3)nc1N[C@H](CO)c1ccccc1] | ethyl 4-[[(1S)-2-hydroxy-1-phenyl- ethyl]amino]-2-[(2-oxo-3,4-dihydro- 1H-quinolin-6-yl)amino)pyrimidine- 5-carboxylate | 448.2 | 448 |
| 47 | 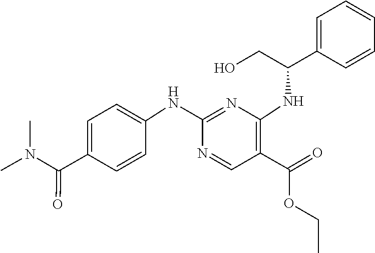 [SMILES: CCOC(=O)c1cnc(Nc2ccc(C(=O)N(C)C)cc2)nc1N[C@H](CO)c1ccccc1] | ethyl 2-[4- (dimethylcarbamoyl)anilino]-4-[[(1S)- 2-hydroxy-1-phenyl- ethyl]amino]pyrimidine-5-carboxylate | 450.2 | 450 |
| 48 | 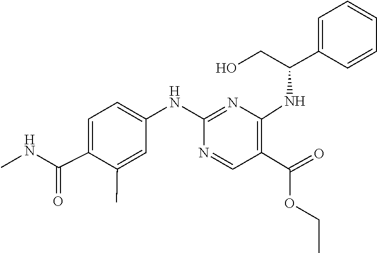 [SMILES: CCOC(=O)c1cnc(Nc2ccc(C(=O)NC)c(C)c2)nc1N[C@H](CO)c1ccccc1] | ethyl 4-[[(1S)-2-hydroxy-1-phenyl- ethyl]amino]-2-[3-methyl-4- (methylcarbamoyl)anilino]pyrimidine- 5-carboxylate | 450.2 | 450 |
| 49 | 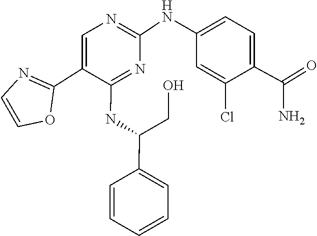 | 2-chloro-4-[[4-[[(1S)-2-hydroxy-1- phenyl-ethyl]amino]-5-oxazol-2-yl- pyrimidin-2-yl]amino]benzamide | 451.1 | 451 |
| 50 | 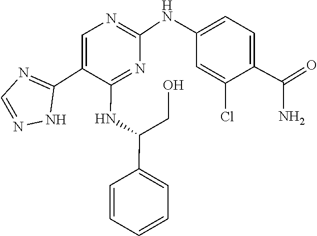 [SMILES: NC(=O)c1ccc(Nc2ncc(-c3ncn[nH]3)c(N[C@H](CO)c3ccccc3)n2)cc1Cl] | 2-chloro-4-[[4-[[(1S)-2-hydroxy-1- phenyl-ethyl]amino]-5-(1H-1,2,4- triazol-5-yl)pyrimidin-2- yl]amino]benzamide | 451.1 | 451 |
| 51 | 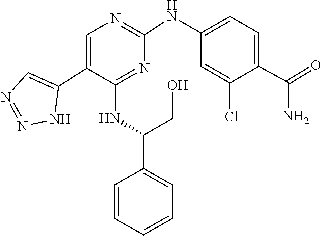 [SMILES: NC(=O)c1ccc(Nc2ncc(-c3cnn[nH]3)c(N[C@H](CO)c3ccccc3)n2)cc1Cl] | 2-chloro-4-[[4-[[(1S)-2-hydroxy-1- phenyl-ethyl]amino]-5-(1H-triazol-5- yl)pyrimidin-2-yl]amino]benzamide | 451.1 | 451 |
| 52 | 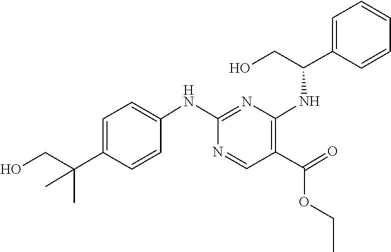 [SMILES: CCOC(=O)c1cnc(Nc2ccc(C(C)(C)CO)cc2)nc1N[C@H](CO)c1ccccc1] | ethyl 2-[4-(2-hydroxy-1,1-dimethyl- ethyl)anilino]-4-[[(2S)-2-hydroxy-1- phenyl-ethyl]amino]pyrimidine-5- carboxylate | 451.2 | 451 |
| 53 | 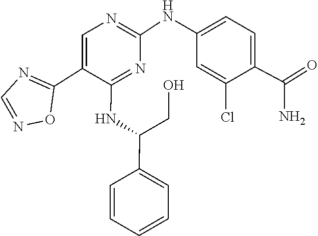 [SMILES: NC(=O)c1ccc(Nc2ncc(-c3ncno3)c(N[C@H](CO)c3ccccc3)n2)cc1Cl] | 2-chloro-4-[[4-[[(1S)-2-hydroxy-1- phenyl-ethyl]amino]-5-(1,2,4- oxadiazol-5-yl)pyrimidin-2- yl]amino]benzamide | 452.1 | 452 |
| 54 | 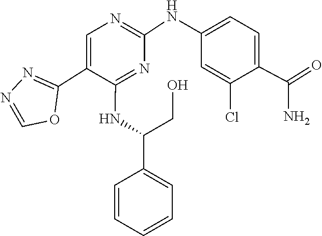 [SMILES: NC(=O)c1ccc(Nc2ncc(-c3nnco3)c(N[C@H](CO)c3ccccc3)n2)cc1Cl] | 2-chloro-4-[[4-[[(1S)-2-hydroxy-1- phenyl-ethyl]amino]-5-(1,3,4- oxadiazol-2-yl)pyrimidin-2- yl]amino]benzamide | 452.1 | 452 |
| 55 | 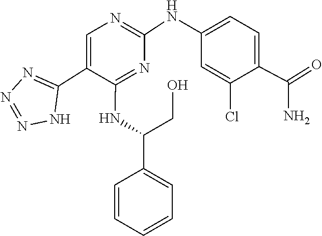 [SMILES: NC(=O)c1ccc(Nc2ncc(-c3nnn[nH]3)c(N[C@H](CO)c3ccccc3)n2)cc1Cl] | 2-chloro-4-[[4-[[(1S)-2-hydroxy-1- phenyl-ethyl]amino]-5-(1H-tetrazol-5- yl)pyrimidin-2-yl]amino]benzamide | 452.1 | 452 |
| 56 | 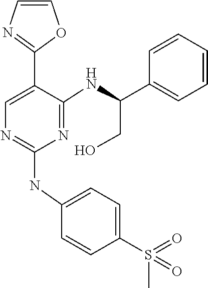 | (2S)-2-[[2-(4-methylsulfonylanilino)- 5-oxazol-2-yl-pyrimidin-4-yl]amino]- 2-phenyl-ethanol | 452.1 | 452 |
| 57 | 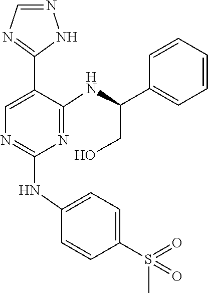 [SMILES: CS(=O)(=O)c1ccc(Nc2ncc(-c3ncn[nH]3)c(N[C@H](CO)c3ccccc3)n2)cc1] | (2S)-2-[[2-(4-methylsulfonylanilino)- 5-(1H-1,2,4-triazol-5-yl)pyrimidin-4- yl]amino]-2-phenyl-ethanol | 452.1 | 452 |
| 58 | 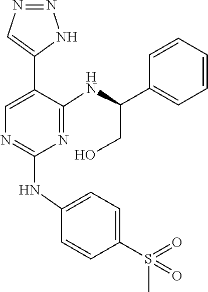 [SMILES: CS(=O)(=O)c1ccc(Nc2ncc(-c3cnn[nH]3)c(N[C@H](CO)c3ccccc3)n2)cc1] | (2S)-2-[[2-(4-methylsulfonylanilino)- 5-(1H-triazol-5-yl)pyrimidin-4- yl]amino]-2-phenyl-ethanol | 452.1 | 452 |
| 59 | 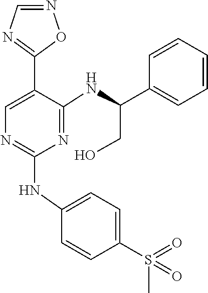 [SMILES: CS(=O)(=O)c1ccc(Nc2ncc(-c3ncno3)c(N[C@H](CO)c3ccccc3)n2)cc1] | (2S)-2-[[2-(4-methysulfonylanilino)- 5-(1,2,4-oxadiazol-5-yl)pyrimidin-4- yl]amino]-2-phenyl-ethanol | 453.1 | 453 |
| 60 | 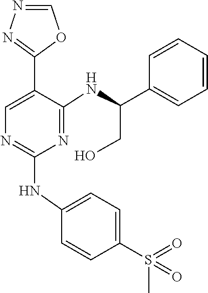 [SMILES: CS(=O)(=O)c1ccc(Nc2ncc(-c3nnco3)c(N[C@H](CO)c3ccccc3)n2)cc1] | (2S)-2-[[2-(4-methylsulfonylanilino)- 5-(1,3,4-oxadiazol-2-yl)pyrimidin-4- yl]amino]-2-phenyl-ethanol | 453.1 | 453 |
| 61 | 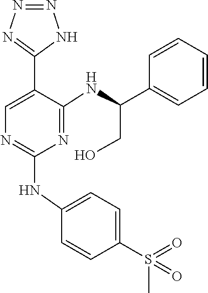 [SMILES: CS(=O)(=O)c1ccc(Nc2ncc(-c3nnn[nH]3)c(N[C@H](CO)c3ccccc3)n2)cc1] | (2S)-2-[[2-(4-methylsulfonylanilino)- 5-(1H-tetrazol-5-yl)pyrimidin-4- yl]amino]-2-phenyl-ethanol | 453.1 | 453 |
| 62 | 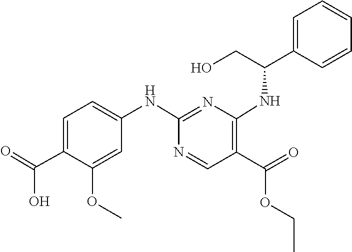 [SMILES: CCOC(=O)c1cnc(Nc2ccc(C(=O)O)c(OC)c2)nc1N[C@H](CO)c1ccccc1] | 4-[[5-ethoxycarbonyl-4-[[(1S)-2- hydroxy-1-phenyl- ethyl]amino]pyrimidin-2-yl]amino]-2- methoxy-benzoic acid | 453.2 | 453 |
| 63 | 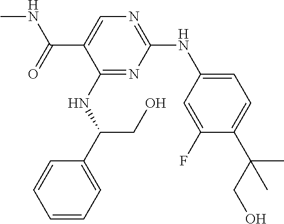 [SMILES: CNC(=O)c1cnc(Nc2ccc(C(C)(C)CO)c(F)c2)nc1N[C@H](CO)c1ccccc1] | 2-[3-fluoro-4-(2-hydroxy-1,1- dimethyl-ethyl)anilino]-4-[[(1S)-2- hydroxy-1-phenyl-ethyl]amino]-N- methyl-pyrimidine-5-carboxamide | 454.2 | 454 |
| 64 | 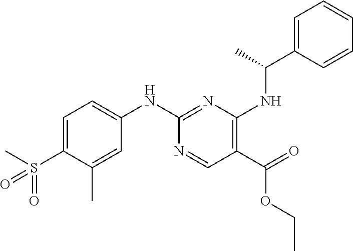 [SMILES: CCOC(=O)c1cnc(Nc2ccc(S(C)(=O)=O)c(C)c2)nc1N[C@H](C)c1ccccc1] | ethyl 2-(3-methyl-4-methylsulfonyl- anilino)-4-[[(1R)-1- phenylethyl]amino]pyrimidine-5- carboxylate | 455.2 | 455 |
| 65 | 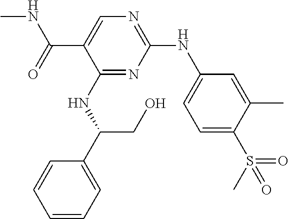 | 4-[[(1S)-2-hydroxy-1-phenyl- ethyl]amino]-N-methyl-2-(3-methyl- 4-methylsulfonyl-anilino)pyrimidine- 5-carboxamide | 456.2 | 456 |
| 66 | 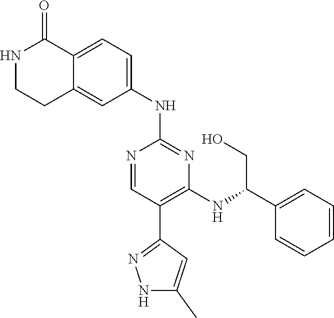 [SMILES: Cc1cc(-c2cnc(Nc3ccc4c(c3)CCNC4=O)nc2N[C@H](CO)c2ccccc2)n[nH]1] | 6-[[4-[[(1S)-2-hydroxy-1-phenyl- ethyl]amino]-5-(5-methyl-1H- pyrazol-3-yl)pyrimidin-2-yl]amino]- 3,4-dihydro-2H-isoquinolin-1-one | 456.2 | 456 |
| 67 | 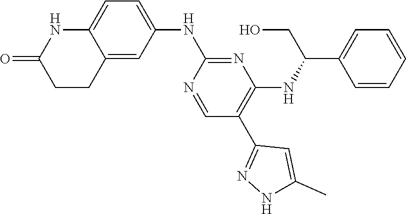 [SMILES: Cc1cc(-c2cnc(Nc3ccc4c(c3)CCC(=O)N4)nc2N[C@H](CO)c2ccccc2)n[nH]1] | 6-[[4-[[(1S)-2-hydroxy-1-phenyl- ethyl]amino]-5-(5-methyl-1H- pyrazol-3-yl)pyrimidin-2-yl]amino]- 3,4-dihydro-1H-quinolin-2-one | 456.2 | 456 |
| 68 | 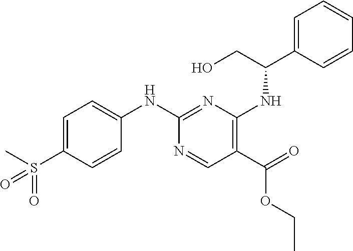 [SMILES: CCOC(=O)c1cnc(Nc2ccc(S(C)(=O)=O)cc2)nc1N[C@H](CO)c1ccccc1] | ethyl 4-[[(1S)-2-hydroxy-1-phenyl- ethyl]amino]-2-(4- methylsulfonylanilino)pyrimidine-5- carboxylate | 457.2 | 457 |
| 69 | 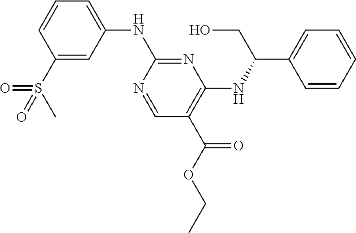 [SMILES: CCOC(=O)c1cnc(Nc2cccc(S(C)(=O)=O)c2)nc1N[C@H](CO)c1ccccc1] | ethyl 4-[[(1S)-2-hydroxy-1-phenyl- ethyl]amino]-2-(3- methysulfonylanilino)pyrimidine-5- carboxylate | 457.2 | 457 |
| 70 | 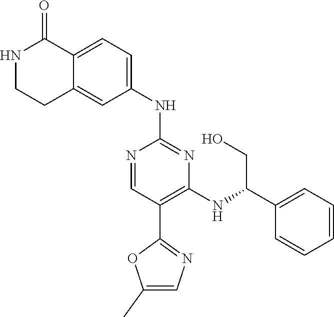 [SMILES: Cc1cnc(-c2cnc(Nc3ccc4c(c3)CCNC4=O)nc2N[C@H](CO)c2ccccc2)o1] | 6-[[4-[[(1S)-2-hydroxy-1-phenyl- ethyl]amino]-5-(5-methyloxazol-2- yl)pyrimidin-2-yl)amino]-3,4- dihydro-2H-isoquinolin-1-one | 457.2 | 457 |
| 71 | 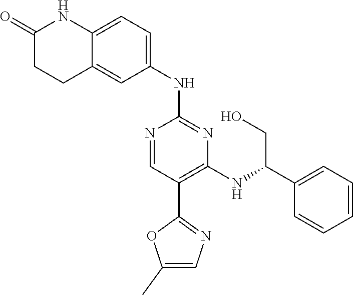 [SMILES: Cc1cnc(-c2cnc(Nc3ccc4c(c3)CCC(=O)N4)nc2N[C@H](CO)c2ccccc2)o1] | 6-[[4-[[(1S)-2-hydroxy-1-phenyl- ethyl]amino]-5-(5-methyloxazol-2- yl)pyrimidin-2-yl]amino]-3,4- dihydro-1H-quinolin-2-one | 457.2 | 457 |
| 72 | 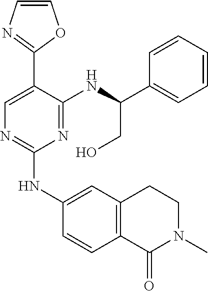 [SMILES: CN1CCc2cc(Nc3ncc(-c4ncco4)c(N[C@H](CO)c4ccccc4)n3)ccc2C1=O] | 6-[[4-[[(1S)-2-hydroxy-1-phenyl- ethyl]amino]-5-oxazol-2-yl- pyrimidin-2-yl]amino]-2-methyl-3,4- dihydroisoquinolin-1-one | 457.2 | 457 |
| 73 | 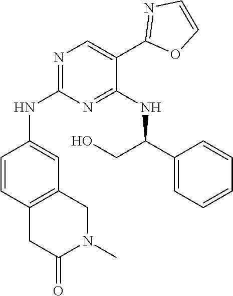 [SMILES: CN1Cc2cc(Nc3ncc(-c4ncco4)c(N[C@H](CO)c4ccccc4)n3)ccc2CC1=O] | 7-[[4-[[(1S)-2-hydroxy-1-phenyl- ethyl]amino]-5-oxazol-2-yl- pyrimidin-2-yl]amino]-2-methyl-1,4- dihydroisoquinolin-3-one | 457.2 | 457 |
| 74 | 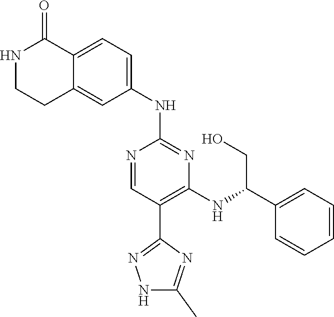 [SMILES: Cc1nc(-c2cnc(Nc3ccc4c(c3)CCNC4=O)nc2N[C@H](CO)c2ccccc2)n[nH]1] | 6-[[4-[[(1S)-2-hydroxy-1-phenyl- ethyl]amino]-5-(5-methyl-1H-1,2,4- triazol-3-yl)pyrimidin-2-yl]amino]- 3,4-dihydro-2H-isoquinolin-1-one | 457.2 | 457 |
| 75 | 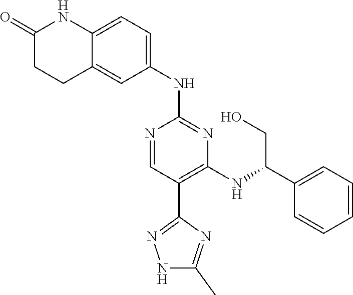 [SMILES: Cc1nc(-c2cnc(Nc3ccc4c(c3)CCC(=O)N4)nc2N[C@H](CO)c2ccccc2)n[nH]1] | 6-[[4-[[(1S)-2-hydroxy-1-phenyl- ethyl]amino]-5-(5-methyl-1H-1,2,4- triazol-3-yl)pyrimidin-2-yl]amino]- 3,4-dihydro-1H-quinolin-2-one | 457.2 | 457 |
| 76 | 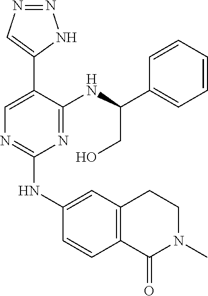 [SMILES: CN1CCc2cc(Nc3ncc(-c4cnn[nH]4)c(N[C@H](CO)c4ccccc4)n3)ccc2C1=O] | 6-[[4-[[(1S)-2-hydroxy-1-phenyl- ethyl]amino]-5-(1H-triazol-5- yl)pyrimidin-2-yl]amino]-2-methyl- 3,4-dihydroisoquinolin-1-one | 457.2 | 457 |
| 77 | 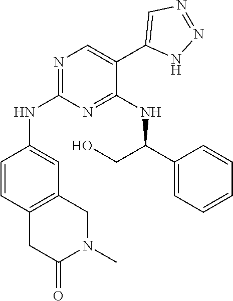 [SMILES: CN1Cc2cc(Nc3ncc(-c4cnn[nH]4)c(N[C@H](CO)c4ccccc4)n3)ccc2CC1=O] | 7-[[4-[[(1S)-2-hydroxy-1-phenyl- ethyl]amino]-5-(1H-triazol-5- yl)pyrimidin-2-yl]amino]-2-methyl- 1,4-dihydroisoquinolin-3-one | 457.2 | 457 |
| 78 | 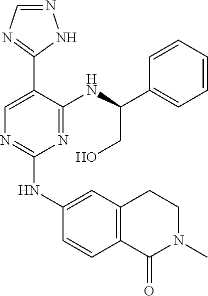 [SMILES: CN1CCc2cc(Nc3ncc(-c4ncn[nH]4)c(N[C@H](CO)c4ccccc4)n3)ccc2C1=O] | 6-[[4-[[(1S)-2-hydroxy-1-phenyl- ethyl]amino]-5-(1H-1,2,4-triazol-5- yl)pyrimidin-2-yl]amino]-2-methyl- 3,4-dihydroisoquinolin-1-one | 457.2 | 457 |
| 79 | 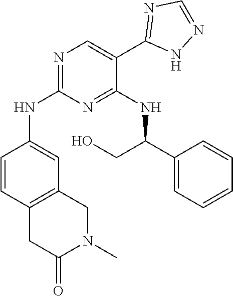 [SMILES: CN1Cc2cc(Nc3ncc(-c4ncn[nH]4)c(N[C@H](CO)c4ccccc4)n3)ccc2CC1=O] | 7-[[4-[[(1S)-2-hydroxy-1-phenyl- ethyl]amino]-5-(1H-1,2,4-triazol-5- yl)pyrimidin-2-yl]amino]-2-methyl- 1,4-dihydroisoquinolin-3-one | 457.2 | 457 |
| 80 | 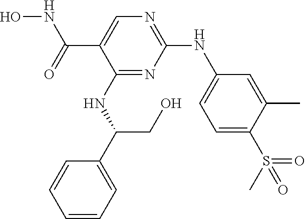 [SMILES: Cc1cc(Nc2ncc(C(=O)NO)c(N[C@H](CO)c3ccccc3)n2)ccc1S(C)(=O)=O] | 4-[[(1S)-2-hydroxy-1-phenyl- ethyl]amino]-2-(3-methyl-4- methylsulfonyl-anilino)pyrimidine-5- carbohydroxamic acid | 458.1 | 458 |
| 81 | 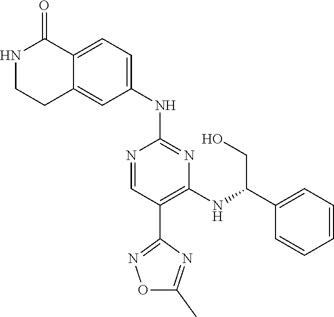 [SMILES: Cc1nc(-c2cnc(Nc3ccc4c(c3)CCNC4=O)nc2N[C@H](CO)c2ccccc2)no1] | 6-[[4-[[(1S)-2-hydroxy-1-phenyl- ethyl]amino]-5-(5-methyl-1,2,4- oxadiazol-3-yl)pyrimidin-2- yl]amino]-3,4-dihydro-2H- isoquinolin-1-one | 458.2 | 458 |
| 82 | 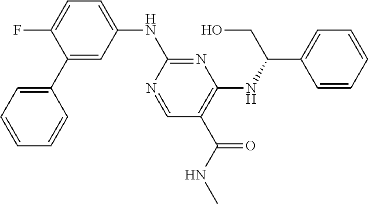 [SMILES: CNC(=O)c1cnc(Nc2ccc(F)c(-c3ccccc3)c2)nc1N[C@H](CO)c1ccccc1] | 2-(4-fluoro-3-phenyl-anilino)-4- [[(1S)-2-hydroxy-1-phenyl- ethyl]amino]-N-methyl-pyrimidine-5- carboxamide | 458.2 | 458 |
| 83 | 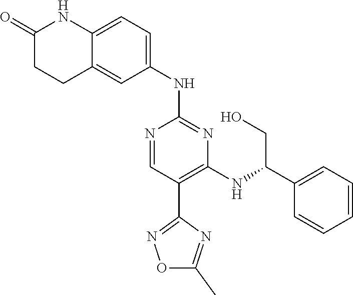 [SMILES: Cc1nc(-c2cnc(Nc3ccc4c(c3)CCC(=O)N4)nc2N[C@H](CO)c2ccccc2)no1] | 6-[[4-[[(1S)-2-hydroxy-1-phenyl- ethyl]amino]-5-(5-methyl-1,2,4- oxadiazol-3-yl)pyrimidin-2- yl]amino]-3,4-dihydro-1H-quinolin-2- one | 458.2 | 458 |
| 84 | 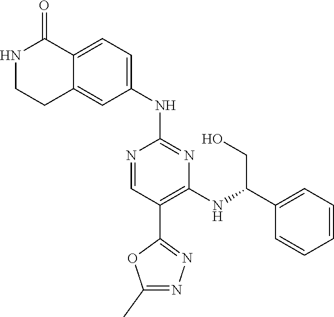 [SMILES: Cc1nnc(-c2cnc(Nc3ccc4c(c3)CCNC4=O)nc2N[C@H](CO)c2ccccc2)o1] | 6-[[4-[[(1S)-2-hydroxy-1-phenyl- ethyl]amino]-5-(5-methyl-1,3,4- oxadiazol-2-yl)pyrimidin-2- yl]amino]-3,4-dihydro-2H- isoquinolin-1-one | 458.2 | 458 |
| 85 | 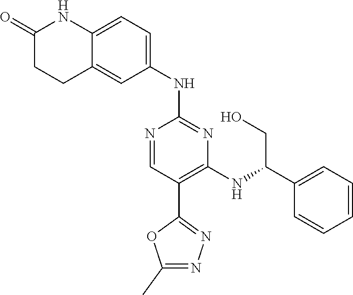 [SMILES: Cc1nnc(-c2cnc(Nc3ccc4c(c3)CCC(=O)N4)nc2N[C@H](CO)c2ccccc2)o1] | 6-[[4-[[(1S)-2-hydroxy-1-phenyl- ethyl]amino]-5-(5-methyl-1,3,4- oxadiazol-2-yl)pyrimidin-2- yl]amino]-3,4-dihydro-1H-quinolin-2- one | 458.2 | 458 |
| 86 | 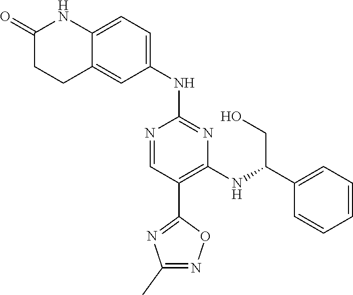 [SMILES: Cc1noc(-c2cnc(Nc3ccc4c(c3)CCC(=O)N4)nc2N[C@H](CO)c2ccccc2)n1] | 6-[[4-[[(1S)-2-hydroxy-1-phenyl- ethyl]amino]-5-(3-methyl-1,2,4- oxadiazol-5-yl)pyrimidin-2- yl]amino]-3,4-dihydro-1H-quinolin-2- one | 458.2 | 458 |
| 87 | 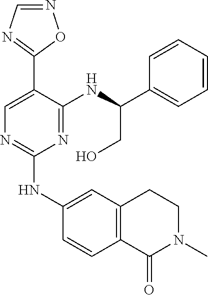 [SMILES: CN1CCc2cc(Nc3ncc(-c4ncno4)c(N[C@H](CO)c4ccccc4)n3)ccc2C1=O] | 6-[[4-[[(1S)-2-hydroxy-1-phenyl- ethyl]amino]-5-(1,2,4-oxadiazol-5- yl)pyrimidin-2-yl]amino]-2-methyl- 3,4-dihydroisoquinolin-1-one | 458.2 | 458 |
| 88 | 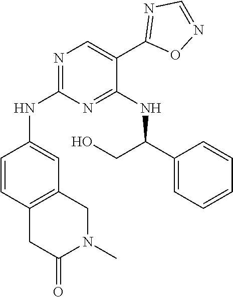 [SMILES: CN1Cc2cc(Nc3ncc(-c4ncno4)c(N[C@H](CO)c4ccccc4)n3)ccc2CC1=O] | 7-[[4-[[(1S)-2-hydroxy-1-phenyl- ethyl]amino]-5-(1,2,4-oxadiazol-5- yl)pyrimidin-2-yl]amino]-2-methyl- 1,4-dihydroisoquinolin-3-one | 458.2 | 458 |
| 89 | 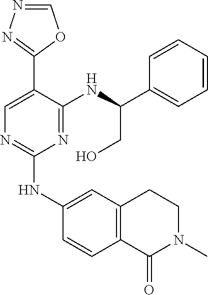 [SMILES: CN1CCc2cc(Nc3ncc(-c4nnco4)c(N[C@H](CO)c4ccccc4)n3)ccc2C1=O] | 6-[[4-[[(1S)-2-hydroxy-1-phenyl- ethyl]amino]-5-(1,3,4-oxadiazol-2- yl)pyrimidin-2-yl]amino]-2-methyl- 3,4-dihydroisoquinolin-1-one | 458.2 | 458 |
| 90 | 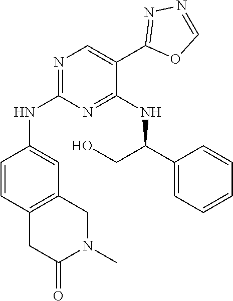 [SMILES: CN1Cc2cc(Nc3ncc(-c4nnco4)c(N[C@H](CO)c4ccccc4)n3)ccc2CC1=O] | 7-[[4-[[(1S)-2-hydroxy-1-phenyl- ethyl]amino]-5-(1,3,4-oxadiazol-2- yl)pyrimidin-2-yl]amino]-2-methyl- 1,4-dihydroisoquinolin-3-one | 458.2 | 458 |
| 91 | 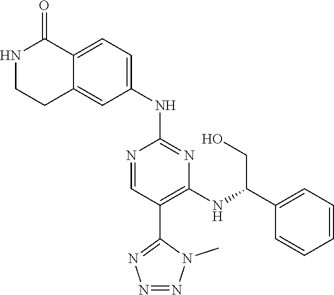 [SMILES: Cn1nnnc1-c1cnc(Nc2ccc3c(c2)CCNC3=O)nc1N[C@H](CO)c1ccccc1] | 6-[[4-[[(1S)-2-hydroxy-1-phenyl- ethyl]amino]-5-(1-methyltetrazol-5- yl)pyrimidin-2-yl]amino]-3,4- dihydro-2H-isoquinolin-1-one | 458.2 | 458 |
| 92 | 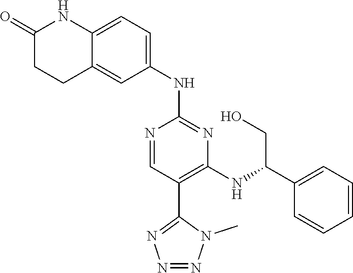 [SMILES: Cn1nnnc1-c1cnc(Nc2ccc3c(c2)CCC(=O)N3)nc1N[C@H](CO)c1ccccc1] | 6-[[4-[[(1S)-2-hydroxy-1-phenyl- ethyl]amino]-5-(1-methyltetrazol-5- yl)pyrimidin-2-yl]amino]-3,4- dihydro-1H-quinolin-2-one | 458.2 | 458 |
| 93 | 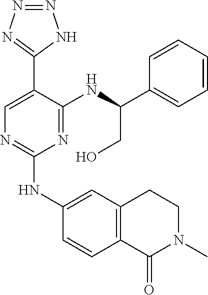 [SMILES: CN1CCc2cc(Nc3ncc(-c4nnn[nH]4)c(N[C@H](CO)c4ccccc4)n3)ccc2C1=O] | 6-[[4-[[(1S)-2-hydroxy-1-phenyl- ethyl]amino]-5-(1H-tetrazol-5- yl)pyrimidin-2-yl]amino]-2-methyl- 3,4-dihydroisoquinolin-1-one | 458.2 | 458 |
| 94 | 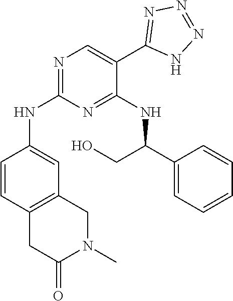 [SMILES: CN1Cc2cc(Nc3ncc(-c4nnn[nH]4)c(N[C@H](CO)c4ccccc4)n3)ccc2CC1=O] | 7-[[4-[[(1S)-2-hydroxy-1-phenyl- ethyl]amino]-5-(1H-tetrazol-5- yl)pyrimidin-2-yl]amino]-2-methyl- 1,4-dihydroisoquinolin-3-one | 458.2 | 458 |
| 95 | 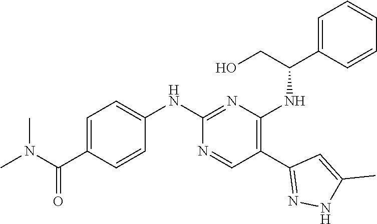 [SMILES: Cc1cc(-c2cnc(Nc3ccc(C(=O)N(C)C)cc3)nc2N[C@H](CO)c2ccccc2)n[nH]1] | 4-[[4-[[(1S)-2-hydroxy-1-phenyl- ethyl]amino]-5-(5-methyl-1H- pyrazol-3-yl)pyrimidin-2-yl]amino]- N,N-dimethyl-benzamide | 458.2 | 458 |
| 96 | 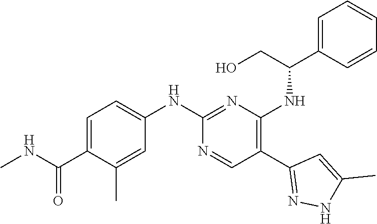 [SMILES: CNC(=O)c1ccc(Nc2ncc(-c3cc(C)[nH]n3)c(N[C@H](CO)c3ccccc3)n2)cc1C] | 4-[[4-[[(1S)-2-hydroxy-1-phenyl- ethyl]amino]-5-(5-methyl-1H- pyrazol-3-yl)pyrimidin-2-yl]amino]- N,2-dimethyl-benzamide | 458.2 | 458 |
| 97 | 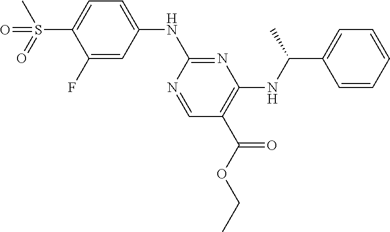 [SMILES: CCOC(=O)c1cnc(Nc2ccc(S(C)(=O)=O)c(F)c2)nc1N[C@H](C)c1ccccc1] | ethyl 2-(3-fluoro-4-methylsulfonyl- anilino)-4-[[(1R)-1- phenylethyl]amino]pyrimidine-5- carboxylate | 459.2 | 459 |
| 98 | 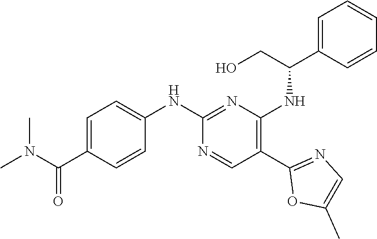 [SMILES: Cc1cnc(-c2cnc(Nc3ccc(C(=O)N(C)C)cc3)nc2N[C@H](CO)c2ccccc2)o1] | 4-[[4-[[(1S)-2-hydroxy-1-phenyl- ethyl]amino]-5-(5-methyloxazol-2- yl)pyrimidin-2-yl]amino]-N,N- dimethyl-benzamide | 459.2 | 459 |
| 99 | 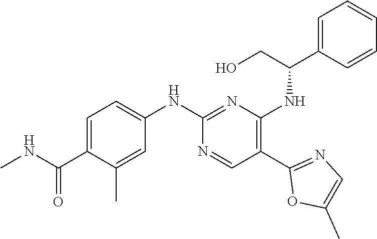 [SMILES: CNC(=O)c1ccc(Nc2ncc(-c3ncc(C)o3)c(N[C@H](CO)c3ccccc3)n2)cc1C] | 4-[[4-[[(1S)-2-hydroxy-1-phenyl- ethyl]amino]-5-(5-methyloxazol-2- yl)pyrimidin-2-yl]amino]-N,2- dimethyl-benzamide | 459.2 | 459 |
| 100 | 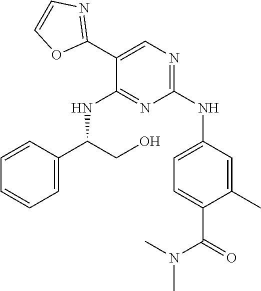 [SMILES: Cc1cc(Nc2ncc(-c3ncco3)c(N[C@H](CO)c3ccccc3)n2)ccc1C(=O)N(C)C] | 4-[[4-[[(1S)-2-hydroxy-1-phenyl- ethyl]amino]-5-oxazol-2-yl- pyrimidin-2-yl]amino]-N,N,2- trimethyl-benzamide | 459.2 | 459 |
| 101 | 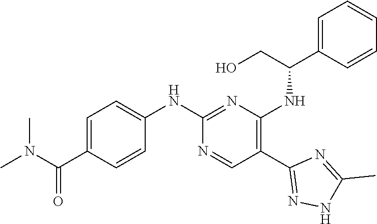 [SMILES: Cc1nc(-c2cnc(Nc3ccc(C(=O)N(C)C)cc3)nc2N[C@H](CO)c2ccccc2)n[nH]1] | 4-[[4-[[(1S)-2-hydroxy-1-phenyl- ethyl]amino]-5-(5-methyl-1H-1,2,4- triazol-3-yl)pyrimidin-2-yl]amino]- N,N-dimethyl-benzamide | 459.2 | 459 |
| 102 | 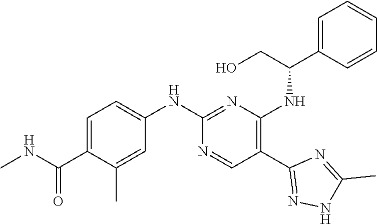 [SMILES: CNC(=O)c1ccc(Nc2ncc(-c3n[nH]c(C)n3)c(N[C@H](CO)c3ccccc3)n2)cc1C] | 4-[[4-[[(1S)-2-hydroxy-1-phenyl- ethyl]amino]-5-(5-methyl-1H-1,2,4- triazol-3-yl)pyrimidin-2-yl)]amino]- N,2-dimethyl-benzamide | 459.2 | 459 |
| 103 | 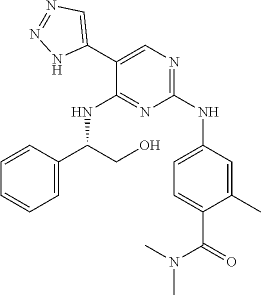 [SMILES: Cc1cc(Nc2ncc(-c3cnn[nH]3)c(N[C@H](CO)c3ccccc3)n2)ccc1C(=O)N(C)C] | 4-[[4-[[(1S)-2-hydroxy-1-phenyl- ethyl]amino]-5-(1H-triazol-5- yl)pyrimidin-2-yl]amino]-N,N,2- trimethyl-benzamide | 459.2 | 459 |
| 104 | 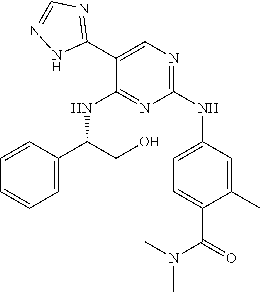 [SMILES: Cc1cc(Nc2ncc(-c3ncn[nH]3)c(N[C@H](CO)c3ccccc3)n2)ccc1C(=O)N(C)C] | 4-[[4-[[(1S)-2-hydroxy-1-phenyl- ethyl]amino]-5-(1H-1,2,4-triazol-5- yl)pyrimidin-2-yl]amino]-N,N,2- trimethyl-benzamide | 459.2 | 459 |
| 105 | 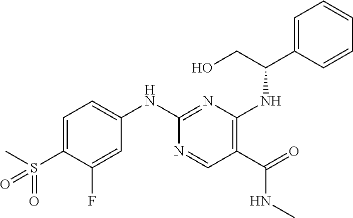 [SMILES: CNC(=O)c1cnc(Nc2ccc(S(C)(=O)=O)c(F)c2)nc1N[C@H](CO)c1ccccc1] | 2-(3-fluoro-4-methylsulfonyl- anilino)-4-[[(1S)-2-hydroxy-1-phenyl- ethyl]amino]-N-methyl-pyrimidin-5- carboxamide | 460.1 | 460 |
| 106 | 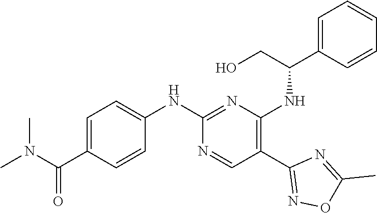 [SMILES: Cc1nc(-c2cnc(Nc3ccc(C(=O)N(C)C)cc3)nc2N[C@H](CO)c2ccccc2)no1] | 4-[[4-[[(1S)-2-hydroxy-1-phenyl- ethyl]amino]-5-(5-methyl-1,2,4- oxadiazol-3-yl)pyrimidin-2- yl]amino]-N,N-dimethyl-benzamide | 460.2 | 460 |
| 107 | 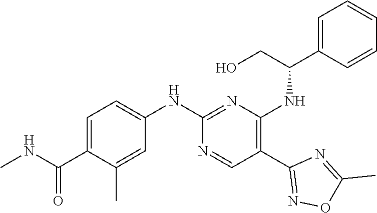 [SMILES: CNC(=O)c1ccc(Nc2ncc(-c3noc(C)n3)c(N[C@H](CO)c3ccccc3)n2)cc1C] | 4-[[4-[[(1S)-2-hydroxy-1-phenyl- ethyl]amino]-5-(5-methyl-1,2,4- oxadiazol-3-yl)pyrimidin-2- yl]amino]-N,2-dimethyl-benzamide | 460.2 | 460 |
| 108 | 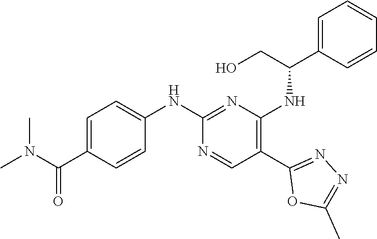 [SMILES: Cc1nnc(-c2cnc(Nc3ccc(C(=O)N(C)C)cc3)nc2N[C@H](CO)c2ccccc2)o1] | 4-[[4-[[(1S)-2-hydroxy-1-phenyl- ethyl]amino]-5-(5-methyl-1,3,4- oxadiazol-2-yl)pyrimidin-2- yl]amino]-N,N-dimethyl-benzamide | 460.2 | 460 |
| 109 | 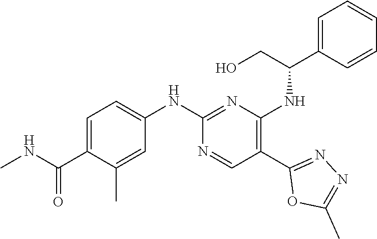 [SMILES: CNC(=O)c1ccc(Nc2ncc(-c3nnc(C)o3)c(N[C@H](CO)c3ccccc3)n2)cc1C] | 4-[[4-[[(1S)-2-hydroxy-1-phenyl- ethyl]amino]-5-(5-methyl-1,3,4- oxadiazol-2-yl)pyrimidin-2- yl]amino]-N,2-dimethyl-benzamide | 460.2 | 460 |
| 110 | 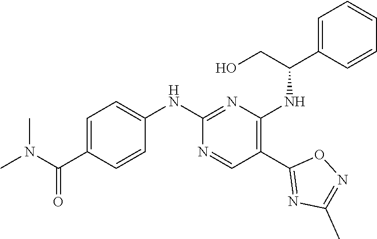 [SMILES: Cc1noc(-c2cnc(Nc3ccc(C(=O)N(C)C)cc3)nc2N[C@H](CO)c2ccccc2)n1] | 4-[[4-[[(1S)-2-hydroxy-1-phenyl- ethyl]amino]-5-(3-methyl-1,2,4- oxadiazol-5-yl)pyrimidin-2- yl]amino]-N,N-dimethyl-benzamide | 460.2 | 460 |
| 111 | 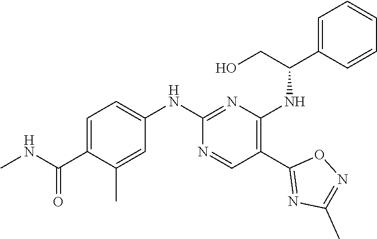 [SMILES: CNC(=O)c1ccc(Nc2ncc(-c3nc(C)no3)c(N[C@H](CO)c3ccccc3)n2)cc1C] | 4-[[4-[[(1S)-2-hydroxy-1-phenyl- ethyl]amino]-5-(3-methyl-1,2,4- oxadiazol-5-yl)pyrimidin-2- yl]amino]-N,2-dimethyl-benzamide | 460.2 | 460 |
| 112 | 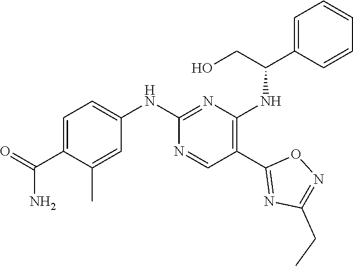 [SMILES: CCc1noc(-c2cnc(Nc3ccc(C(N)=O)c(C)c3)nc2N[C@H](CO)c2ccccc2)n1] | 4-[[5-(3-ethyl-1,2,4-oxadiazol-5-yl)- 4-[[(1S)-2-hydroxy-1-phenyl- ethyl]amino]pyrimidin-2-yl]amino]-2- methyl-benzamide | 460.2 | 460 |
| 113 | 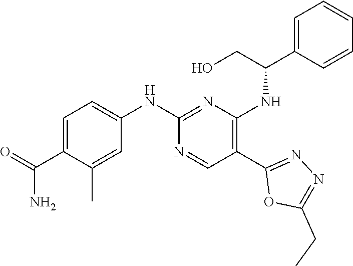 [SMILES: CCc1nnc(-c2cnc(Nc3ccc(C(N)=O)c(C)c3)nc2N[C@H](CO)c2ccccc2)o1] | 4-[[5-(5-ethyl-1,3,4-oxadiazol-2-yl)- 4-[[(1S)-2-hydroxy-1-phenyl- ethyl]amino]pyrimidin-2-yl]amino]-2- methyl-benzamide | 460.2 | 460 |
| 114 | 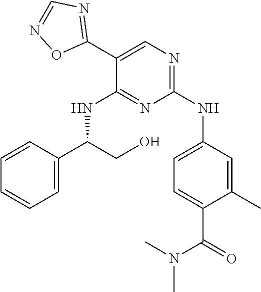 [SMILES: Cc1cc(Nc2ncc(-c3ncno3)c(N[C@H](CO)c3ccccc3)n2)ccc1C(=O)N(C)C] | 4-[[4-[[(1S)-2-hydroxy-1-phenyl- ethyl]amino]-5-(1,2,4-oxadiazol-5- yl)pyrimidin-2-yl]amino]-N,N,2- trimethyl-benzamide | 460.2 | 460 |
| 115 | 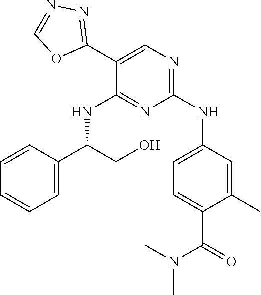 [SMILES: Cc1cc(Nc2ncc(-c3nnco3)c(N[C@H](CO)c3ccccc3)n2)ccc1C(=O)N(C)C] | 4-[[4-[[(1S)-2-hydroxy-1-phenyl- ethyl]amino]-5-(1,3,4-oxadiazol-2- yl)pyrimidin-2-yl]amino]-N,N,2- trimethyl-benzamide | 460.2 | 460 |
| 116 | 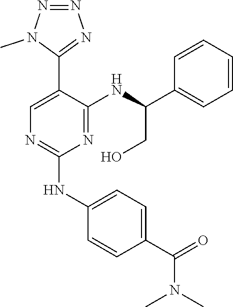 [SMILES: CN(C)C(=O)c1ccc(Nc2ncc(-c3nnnn3C)c(N[C@H](CO)c3ccccc3)n2)cc1] | 4-[[4-[[(1S)-2-hydroxy-1-phenyl- ethyl]amino]-5-(1-methyltetrazol-5- yl)pyrimidin-2-yl]amino]-N,N- dimethyl-benzamide | 460.2 | 460 |
| 117 | 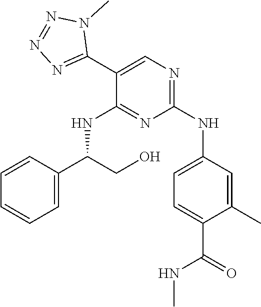 [SMILES: CNC(=O)c1ccc(Nc2ncc(-c3nnnn3C)c(N[C@H](CO)c3ccccc3)n2)cc1C] | 4-[[4-[[(1S)-2-hydroxy-1-phenyl- ethyl]amino]-5-(1-methyltetrazol-5- yl)pyrimidin-2-yl]amino]-N,2- dimethyl-benzamide | 460.2 | 460 |
| 118 | 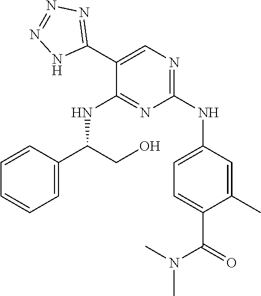 [SMILES: Cc1cc(Nc2ncc(-c3nnn[nH]3)c(N[C@H](CO)c3ccccc3)n2)ccc1C(=O)N(C)C] | 4-[[4-[[(1S)-2-hydroxy-1-phenyl- ethyl]amino]-5-(1H-tetrazol-5- yl)pyrimidin-2-yl]amino]-N,N,2- trimethyl-benzamide | 460.2 | 460 |
| 119 | 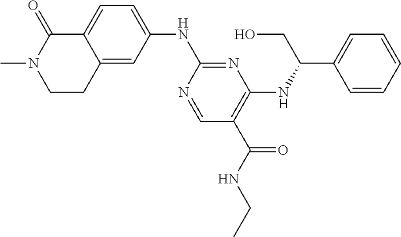 [SMILES: CCNC(=O)c1cnc(Nc2ccc3c(c2)CCN(C)C3=O)nc1N[C@H](CO)c1ccccc1] | N-ethyl-4-[[(1S)-2-hydroxy-1-phenyl- ethyl]amino]-2-[(2-methyl-1-oxo-3,4- dihydroisoquinolin-6- yl)amino]pyrimidine-5-carboxamide | 461.2 | 461 |
| 120 | 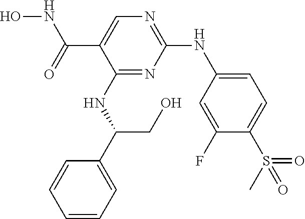 [SMILES: CS(=O)(=O)c1ccc(Nc2ncc(C(=O)NO)c(N[C@H](CO)c3ccccc3)n2)cc1F] | 2-(3-fluoro-4-methylsulfonyl- anilino)-4-[[(1S)-2-hydroxy-1-phenyl- ethyl]amino]pyrimidine-5- carbohydroxamic acid | 462.1 | 462 |
| 121 | 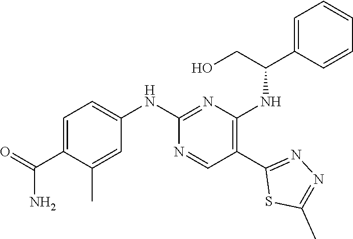 [SMILES: Cc1nnc(-c2cnc(Nc3ccc(C(N)=O)c(C)c3)nc2N[C@H](CO)c2ccccc2)s1] | 4-[[4-[[(1S)-2-hydroxy-1-phenyl- ethyl]amino]-5-(5-methyl-1,3,4- thiadiazol-2-yl)pyrimidin-2- yl]amino]-2-methyl-benzamide | 462.2 | 462 |
| 122 | 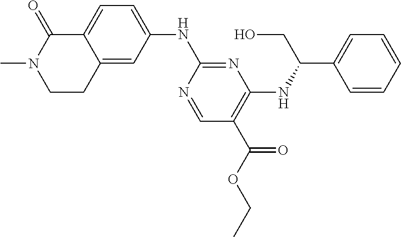 [SMILES: CCOC(=O)c1cnc(Nc2ccc3c(c2)CCN(C)C3=O)nc1N[C@H](CO)c1ccccc1] | ethyl 4-[[(1S)-2-hydroxy-1-phenyl- ethyl]amino]-2-[(2-methyl-1-oxo-3,4- dihydroisoquinolin-6- yl)amino]pyrimidine-5-carboxylate | 462.2 | 462 |
| 123 | 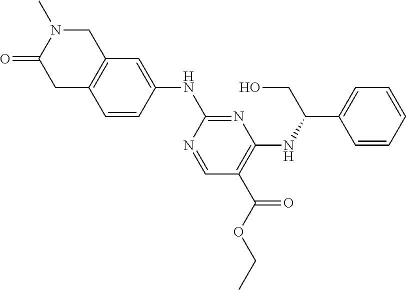 [SMILES: CCOC(=O)c1cnc(Nc2ccc3c(c2)CN(C)C(=O)C3)nc1N[C@H](CO)c1ccccc1] | ethyl 4-[[(1S)-2-hydroxy-1-phenyl- ethyl]amino]-2-[(2-methyl-3-oxo-1,4- dihydroisoquinolin-7- yl)amino]pyrimidine-5-carboxylate | 462.2 | 462 |
| 124 | 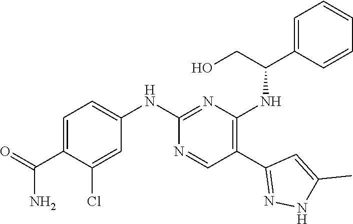 [SMILES: Cc1cc(-c2cnc(Nc3ccc(C(N)=O)c(Cl)c3)nc2N[C@H](CO)c2ccccc2)n[nH]1] | 2-chloro-4-[[4-[[(1S)-2-hydroxy-1- phenyl-ethyl]amino]-5-(5-methyl-1H- pyrazol-3-yl)pyrimidin-2- yl]amino]benzamide | 464.2 | 464 |
| 125 | 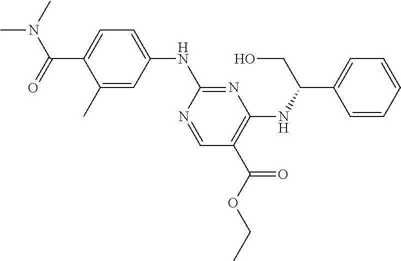 [SMILES: CCOC(=O)c1cnc(Nc2ccc(C(=O)N(C)C)c(C)c2)nc1N[C@H](CO)c1ccccc1] | ethyl 2-[4-(dimethylcarbamoyl)-3- methyl-anilino]-4-[[(1S)-2-hydroxy-1- phenyl-ethyl]amino]pyrimidine-5- carboxylate | 464.2 | 464 |
| 126 | 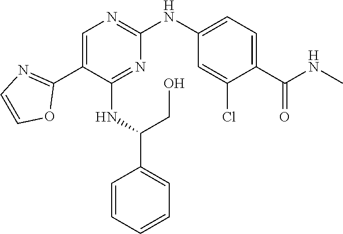 [SMILES: CNC(=O)c1ccc(Nc2ncc(-c3ncco3)c(N[C@H](CO)c3ccccc3)n2)cc1Cl] | 2-chloro-4-[[4-[[(1S)-2-hydroxy-1- phenyl-ethyl]amino]-5-oxazol-2-yl- pyrimidin-2-yl]amino]-N-methyl- benzamide | 465.1 | 465 |
| 127 | 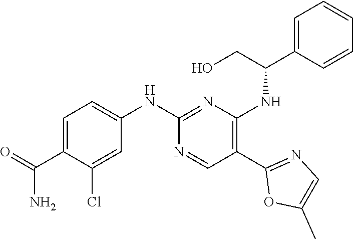 [SMILES: Cc1cnc(-c2cnc(Nc3ccc(C(N)=O)c(Cl)c3)nc2N[C@H](CO)c2ccccc2)o1] | 2-chloro-4-[[4-[[(1S)-2-hydroxy-1- phenyl-ethyl]amino]-5-(5- methyloxazol-2-yl)pyrimidin-2- yl]amino]benzamide | 465.1 | 465 |
| 128 | 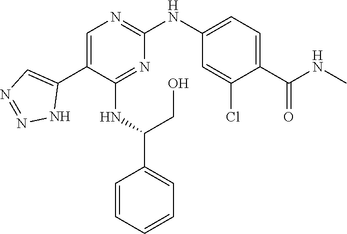 [SMILES: CNC(=O)c1ccc(Nc2ncc(-c3cnn[nH]3)c(N[C@H](CO)c3ccccc3)n2)cc1Cl] | 2-chloro-4-[[4-[[(1S)-2-hydroxy-1- phenyl-ethyl]amino]-5-(1H-triazol-5- yl)pyrimidin-2-yl]amino]-N-methyl- benzamide | 465.2 | 465 |
| 129 | 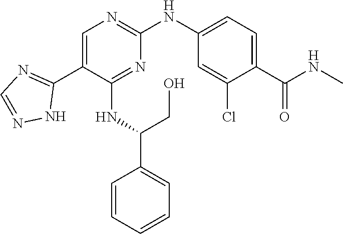 [SMILES: CNC(=O)c1ccc(Nc2ncc(-c3ncn[nH]3)c(N[C@H](CO)c3ccccc3)n2)cc1Cl] | 2-chloro-4-[[4-[[(1S)-2-hydroxy-1- phenyl-ethyl]amino]-5-(1H-1,2,4- triazol-5-yl)pyrimidin-2-yl]amino]-N- methyl-benzamide | 465.2 | 465 |
| 130 | 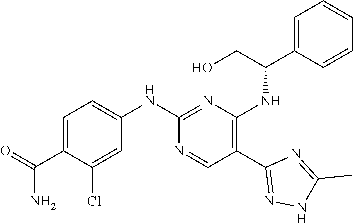 [SMILES: Cc1nc(-c2cnc(Nc3ccc(C(N)=O)c(Cl)c3)nc2N[C@H](CO)c2ccccc2)n[nH]1] | 2-chloro-4-[[4-[[(1S)-2-hydroxy-1- phenyl-ethyl]amino]-5-(5-methyl-1H- 1,2,4-triazol-3-yl)pyrimidin-2- yl]amino]benzamide | 465.2 | 465 |
| 131 | 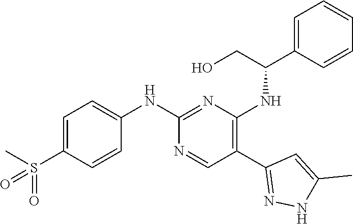 [SMILES: Cc1cc(-c2cnc(Nc3ccc(S(C)(=O)=O)cc3)nc2N[C@H](CO)c2ccccc2)n[nH]1] | (2S)-2-[[5-(5-methyl-1H-pyrazol-3- yl)-2-(4-methylsulfonylanilino)- pyrimidin-4-yl]amino]-2-phenyl- ethanol | 465.2 | 465 |
| 132 | 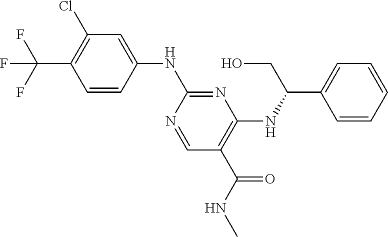 [SMILES: CNC(=O)c1cnc(Nc2ccc(C(F)(F)F)c(Cl)c2)nc1N[C@H](CO)c1ccccc1] | 2-[3-chloro-4- (trifluoromethyl)anilino]-4-[[(1S)-2- hydroxy-1-phenyl-ethyl]amino]-N- methyl-pyrimidine-5-carboxamide | 466.1 | 466 |
| 133 | 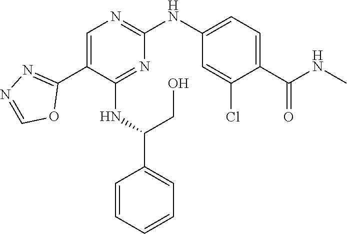 [SMILES: CNC(=O)c1ccc(Nc2ncc(-c3nnco3)c(N[C@H](CO)c3ccccc3)n2)cc1Cl] | 2-chloro-4-[[4-[[(1S)-2-hydroxy-1- phenyl-ethyl]amino]-5-(1,3,4- oxadiazol-2-yl)pyrimidin-2- yl]amino]-N-methyl-benzamide | 466.1 | 466 |
| 134 | 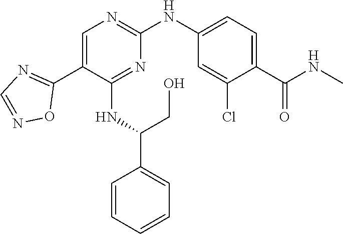 [SMILES: CNC(=O)c1ccc(Nc2ncc(-c3ncno3)c(N[C@H](CO)c3ccccc3)n2)cc1Cl] | 2-chloro-4-[[4-[[(1S)-2-hydroxy-1- phenyl-ethyl]amino]-5-(1,2,4- oxadiazol-5-yl)pyrimidin-2- yl]amino]-N-methyl-benzamide | 466.1 | 466 |
| 135 | 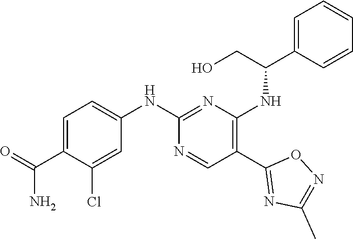 [SMILES: Cc1noc(-c2cnc(Nc3ccc(C(N)=O)c(Cl)c3)nc2N[C@H](CO)c2ccccc2)n1] | 2-chloro-4-[[4-[[(1S)-2-hydroxy-1- phenyl-ethyl]amino]-5-(3-methyl- 1,2,4-oxadiazol-5-yl)pyrimidin-2- yl]amino]benamide | 466.1 | 466 |
| 136 | 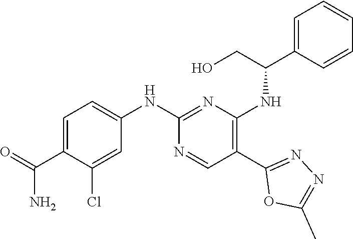 [SMILES: Cc1nnc(-c2cnc(Nc3ccc(C(N)=O)c(Cl)c3)nc2N[C@H](CO)c2ccccc2)o1] | 2-chloro-4-[[4-[[(1S)-2-hydroxy-1- phenyl-ethyl]amino]-5-(5-methyl- 1,3,4-oxadiazol-2-yl)pyrimidin-2- yl]amino]benzamide | 466.1 | 466 |
| 137 | 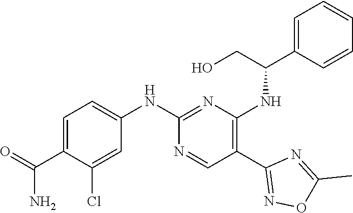 [SMILES: Cc1nc(-c2cnc(Nc3ccc(C(N)=O)c(Cl)c3)nc2N[C@H](CO)c2ccccc2)no1] | 2-chloro-4-[[4-[[(1S)-2-hydroxy-1- phenyl-ethyl]amino]-5-(5-methyl- 1,2,4-oxadiazol-3-yl)pyrimidin-2- yl]amino]benzamide | 466.1 | 466 |
| 138 | 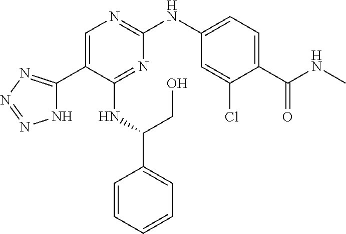 [SMILES: CNC(=O)c1ccc(Nc2ncc(-c3nnn[nH]3)c(N[C@H](CO)c3ccccc3)n2)cc1Cl] | 2-chloro-4-[[4-[[(1S)-2-hydroxy-1- phenyl-ethyl]amino]-5-(1H-tetrazol-5- yl)pyrimidin-2-yl]amino]-N-methyl- benzamide | 466.2 | 466 |
| 139 | 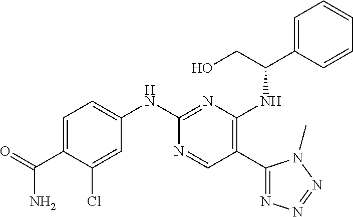 [SMILES: Cn1nnnc1-c1cnc(Nc2ccc(C(N)=O)c(Cl)c2)nc1N[C@H](CO)c1ccccc1] | 2-chloro-4-[[4-[[(1S)-2-hydroxy-1- phenyl-ethyl]amino]-5-(1- methyltetrazol-5-yl)pyrimidin-2- yl]amino]benzamide | 466.2 | 466 |
| 140 | 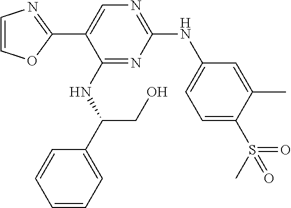 [SMILES: Cc1cc(Nc2ncc(-c3ncco3)c(N[C@H](CO)c3ccccc3)n2)ccc1S(C)(=O)=O] | (2S)-2-[[2-(3-methyl-4- methylsulfonyl-anilino)-5-oxazol-2- yl-pyrimidin-4-yl]amino]-2-phenyl- ethanol | 466.2 | 466 |
| 141 | 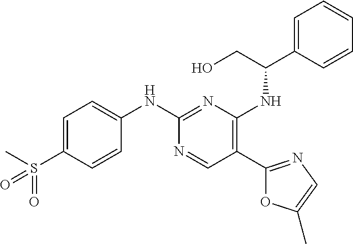 [SMILES: Cc1cnc(-c2cnc(Nc3ccc(S(C)(=O)=O)cc3)nc2N[C@H](CO)c2ccccc2)o1] | (2S)-2-[[5-(5-methyloxazol-2-yl)-2- (4-methylsulfonylanilino)-pyrimidin- 4-yl]amino]-2-phenyl-ethanol | 466.2 | 466 |
| 142 | 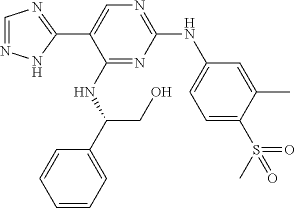 [SMILES: Cc1cc(Nc2ncc(-c3ncn[nH]3)c(N[C@H](CO)c3ccccc3)n2)ccc1S(C)(=O)=O] | (2S)-2-[[2-(3-methyl-4- methylsulfonyl-anilino)-5-(1H-1,2,4- triazol-5-yl)pyrimidin-4-yl]amino]-2- phenyl-ethanol | 466.2 | 466 |
| 143 | 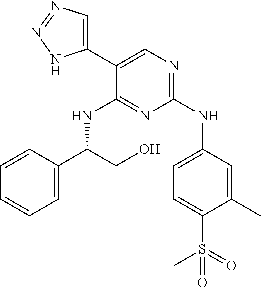 [SMILES: Cc1cc(Nc2ncc(-c3cnn[nH]3)c(N[C@H](CO)c3ccccc3)n2)ccc1S(C)(=O)=O] | (2S)-2-[[2-(3-methyl-4- methylsulfonyl-anilino)-5-(1H-triazol- 5-yl)pyrimidin-4-yl]amino]-2-phenyl- ethanol | 466.2 | 466 |
| 144 | 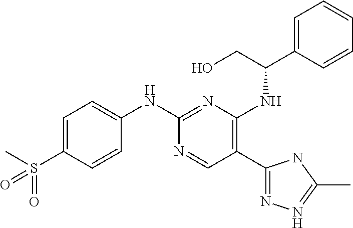 [SMILES: Cc1nnc(-c2cnc(Nc3ccc(S(C)(=O)=O)cc3)nc2N[C@H](CO)c2ccccc2)[nH]1] | (2S)-2-[[2-(4-methylsulfonylanilino)- 5-(5-methyl-1H-1,2,4-triazol-3- yl)pyrimidin-4-yl]amino]-2-phenyl- ethanol | 466.2 | 466 |
| 145 | 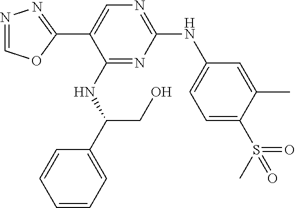 [SMILES: Cc1cc(Nc2ncc(-c3nnco3)c(N[C@H](CO)c3ccccc3)n2)ccc1S(C)(=O)=O] | (2S)-2-[[2-(3-methyl-4- methylsulfonyl-anilino)-5-(1,3,4- oxadiazol-2-yl)pyrimidin-4- yl]amino]-2-phenyl-ethanol | 467.2 | 467 |
| 146 | 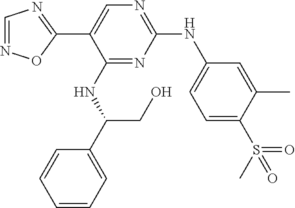 [SMILES: Cc1cc(Nc2ncc(-c3ncno3)c(N[C@H](CO)c3ccccc3)n2)ccc1S(C)(=O)=O] | (2S)-2-[[2-(3-methyl-4- methylsulfonyl-anilino)-5-(1,2,4- oxadiazol-5-yl)pyrimidin-4- yl]amino]-2-phenyl-ethanol | 467.2 | 467 |
| 147 | 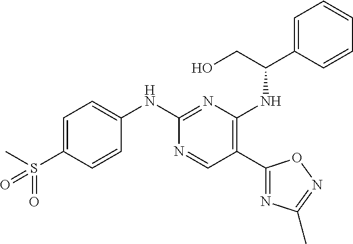 [SMILES: Cc1noc(-c2cnc(Nc3ccc(S(C)(=O)=O)cc3)nc2N[C@H](CO)c2ccccc2)n1] | (2S)-2-[[5-(3-methyl-1,2,4-oxadiazol- 5-yl)-2-(4-methylsulfonylanilino)- pyrimidin-4-yl]amino]-2-phenyl- ethanol | 467.2 | 467 |
| 148 | 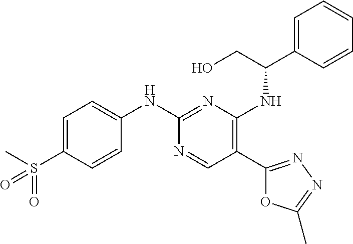 [SMILES: Cc1nnc(-c2cnc(Nc3ccc(S(C)(=O)=O)cc3)nc2N[C@H](CO)c2ccccc2)o1] | (2S)-2-[[5-(5-methyl-1,3,4-oxadiazol- 2-yl)-2-(4-methylsulfonylanilino)- pyrimidin-4-yl]amino]-2-phenyl- ethanol | 467.2 | 467 |
| 149 | 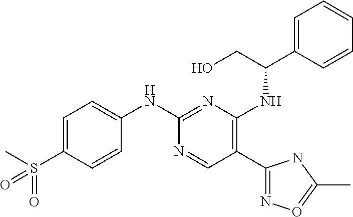 | (2S)-2-[[5-(5-methyl-1,2,4-oxadiazol- 3-yl)-2-(4-methylsulfonylanilino)- pyrimidin-4-yl]amino]-2-phenyl- ethanol | 467.2 | 467 |
| 150 | 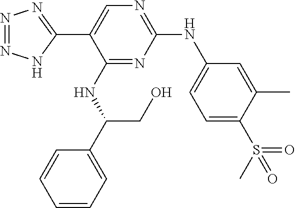 [SMILES: Cc1cc(Nc2ncc(-c3nnn[nH]3)c(N[C@H](CO)c3ccccc3)n2)ccc1S(C)(=O)=O] | (2S)-2-[[2-(3-methyl-4- methylsulfonyl-anilino)-5-(1H- tetrazol-5-yl)pyrimidin-4-yl]amino]- 2-phenyl-ethanol | 467.2 | 467 |
| 151 | 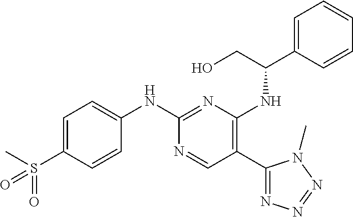 [SMILES: Cn1nnnc1-c1cnc(Nc2ccc(S(C)(=O)=O)cc2)nc1N[C@H](CO)c1ccccc1] | (2S)-2-[[2-(4-methylsulfonylanilino)- 5-(1-methyltetrazol-5-yl)pyrimidin-4- yl]amino]-2-phenyl-ethanol | 467.2 | 467 |
| 152 | 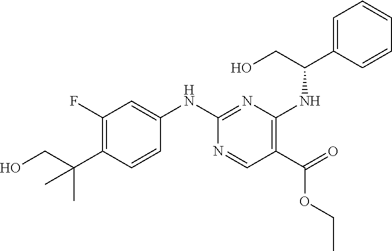 [SMILES: CCOC(=O)c1cnc(Nc2ccc(C(C)(C)CO)c(F)c2)nc1N[C@H](CO)c1ccccc1] | ethyl 2-[3-fluoro-4-(2-hydroxy-1,1- dimethyl-ethyl)anilino]-4-[[(1S)-2- hydroxy-1-phenyl- ethyl]amino]pyrimidine-5-carboxylate | 469.2 | 469 |
| 153 | 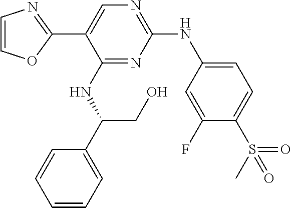 [SMILES: CS(=O)(=O)c1ccc(Nc2ncc(-c3ncco3)c(N[C@H](CO)c3ccccc3)n2)cc1F] | (2S)-2-[[2-(3-fluoro-4- methylsulfonyl-anilino)-5-oxazol-2- yl-pyrimidin-4-yl]amino]-2-phenyl- ethanol | 470.1 | 470 |
| 154 | 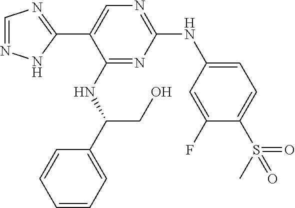 [SMILES: CS(=O)(=O)c1ccc(Nc2ncc(-c3ncn[nH]3)c(N[C@H](CO)c3ccccc3)n2)cc1F] | (2S)-2-[[2-(3-fluoro-4- methylsulfonyl-anilino)-5-(1H-1,2,4- triazol-5-yl)pyrimidin-4-yl]amino]-2- phenyl-ethanol | 470.1 | 470 |
| 155 | 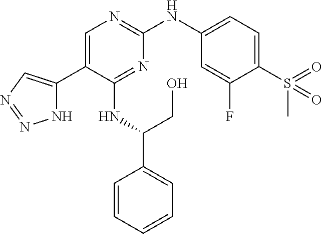 [SMILES: CS(=O)(=O)c1ccc(Nc2ncc(-c3cnn[nH]3)c(N[C@H](CO)c3ccccc3)n2)cc1F] | (2S)-2-[[2-(3-fluoro-4- methylsulfonyl-anilino)-5-(1H-triazol- 5-yl)pyrimidin-4-yl]amino]-2-phenyl- ethanol | 470. | 470 |
| 156 | 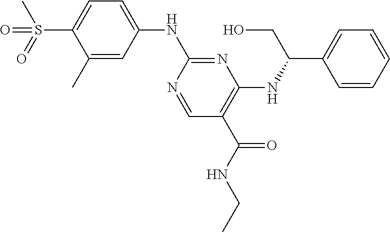 [SMILES: CCNC(=O)c1cnc(Nc2ccc(S(C)(=O)=O)c(C)c2)nc1N[C@H](CO)c1ccccc1] | N-ethyl-4-[[(1S)-2-hydroxy-1-phenyl- ethyl]amino]-2-(3-methyl-4- methylsulfonyl-anilino)pyrimidine-5- carboxamide | 470.2 | 470 |
| 157 | 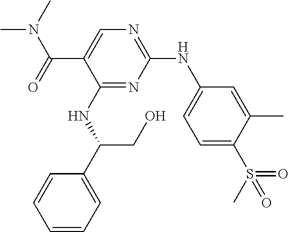 | 4-[[(1S)-2-hydroxy-1-phenyl- ethyl]amino]-N,N-dimethyl-2-(3- methyl-4-methylsulfonyl- anilino)pyrimidine-5-carboxamide | 470.2 | 470 |
| 158 | 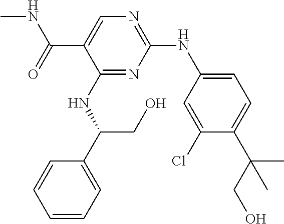 [SMILES: CNC(=O)c1cnc(Nc2ccc(C(C)(C)CO)c(Cl)c2)nc1N[C@H](CO)c1ccccc1] | 2-[3-chloro-4-(2-hydroxy-1,1- dimethyl-ethyl)anilino]-4-[[(1S)-2- hydroxy-1-phenyl-ethyl]amino]-N- methyl-pyrimidine-5-carboxamide | 470.2 | 470 |
| 159 | 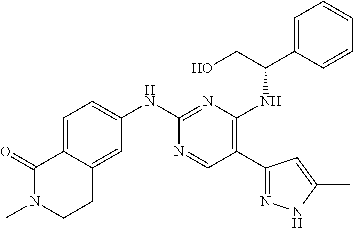 [SMILES: Cc1cc(-c2cnc(Nc3ccc4c(c3)CCN(C)C4=O)nc2N[C@H](CO)c2ccccc2)n[nH]1] | 6-[[4-[[(1S)-2-hydroxy-1-phenyl- ethyl]amino]-5-(5-methyl-1H- pyrazol-3-yl)pyrimidin-2-yl]amino]- 2-methyl-3,4-dihydroisoquinolin-1- one | 470.2 | 470 |
| 160 | 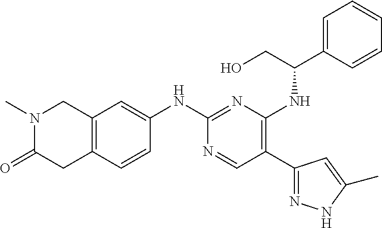 [SMILES: Cc1cc(-c2cnc(Nc3ccc4c(c3)CN(C)C(=O)C4)nc2N[C@H](CO)c2ccccc2)n[nH]1] | 7-[[4-[[(1S)-2-hydroxy-1-phenyl- ethyl]amino]-5-(5-methyl-1H- pyrazol-3-yl)pyrimidin-2-yl]amino]- 2-methyl-1,4-dihydroisoquinolin-3 one | 470.2 | 470 |
| 161 | 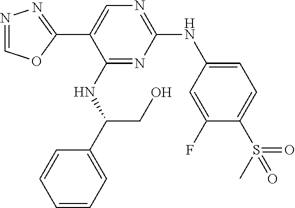 [SMILES: CS(=O)(=O)c1ccc(Nc2ncc(-c3nnco3)c(N[C@H](CO)c3ccccc3)n2)cc1F] | (2S)-2-[[2-(3-fluoro-4- methylsulfonyl-anilino)-5-(1,3,4- oxadiazol-2-yl)pyrimidin-4- yl]amino]-2-phenyl-ethanol | 471.1 | 471 |
| 162 | 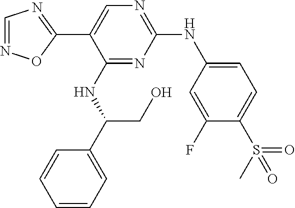 [SMILES: CS(=O)(=O)c1ccc(Nc2ncc(-c3ncno3)c(N[C@H](CO)c3ccccc3)n2)cc1F] | (2S)-2-[[2-(3-fluoro-4- methylsulfonyl-anilino)-5-(1,2,4- oxadiazol-5-yl)pyrimidin-4- yl]amino]-2-phenyl-ethanol | 471.1 | 471 |
| 163 | 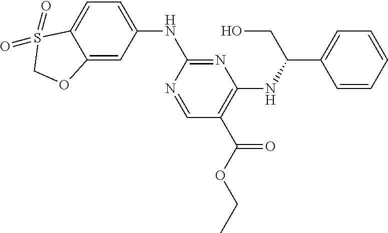 [SMILES: CCOC(=O)c1cnc(Nc2ccc3c(c2)OCS3(=O)=O)nc1N[C@H](CO)c1ccccc1] | ethyl 2-[(3,3-dioxo-1,3lambda6- benzoxathiol-6-yl)amino]-4-[[(1S)-2- hydroxy-1-phenyl- ethyl]amino]pyrimidine-5-carboxylate | 471.1 | 471 |
| 164 | 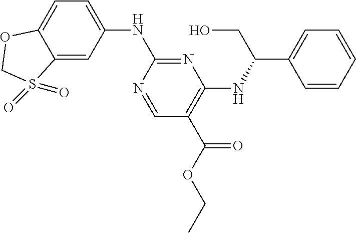 [SMILES: CCOC(=O)c1cnc(Nc2ccc3c(c2)S(=O)(=O)CO3)nc1N[C@H](CO)c1ccccc1] | ethyl 2-[(3,3-dioxo-1,3lambda6- benzoxathiol-5-yl)amio]-4-[[(1S)-2- hydroxy-1-phenyl- ethyl]amino]pyrimidine-5-carboxylate | 471.3 | 471 |
| 165 | 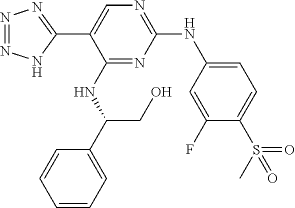 [SMILES: CS(=O)(=O)c1ccc(Nc2ncc(-c3nnn[nH]3)c(N[C@H](CO)c3ccccc3)n2)cc1F] | (2S)-2-[[2-(3-fluoro-4- methylsulfonyl-anilino)-5-(1H- tetrazol-5-yl)pyrimidin-4-yl]amino]- 2-phenyl-ethanol | 471.1 | 471 |
| 166 | 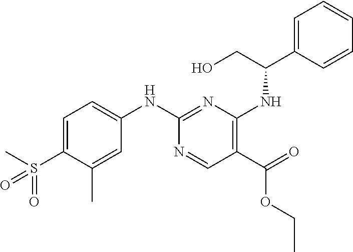 [SMILES: CCOC(=O)c1cnc(Nc2ccc(S(C)(=O)=O)c(C)c2)nc1N[C@H](CO)c1ccccc1] | ethyl 4-[[(1S)-2-hydroxy-1-phenyl- ethyl]amino]-2-(3-methyl-4- methylsulfonyl-anilino)pyrimidine-5- carboxylate | 471.2 | 471 |
| 167 | 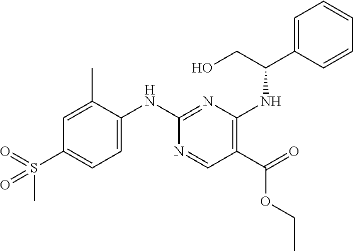 [SMILES: CCOC(=O)c1cnc(Nc2ccc(S(C)(=O)=O)cc2C)nc1N[C@H](CO)c1ccccc1] | ethyl 4-[[(1S)-2-hydroxy-1-phenyl- ethyl]amino]-2-(2-methyl-4- methylsulfonyl-anilino)pyrimidine-5- carboxylate | 471.2 | 471 |
| 168 | 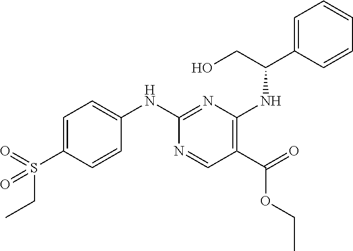 [SMILES: CCOC(=O)c1cnc(Nc2ccc(S(=O)(=O)CC)cc2)nc1N[C@H](CO)c1ccccc1] | ethyl 2-(4-ethylsulfonylanilino)-4- [[(1S)-2-hydroxy-1-phenyl- ethyl]amino]pyrimidine-5-carboxylate | 471.2 | 471 |
| 169 | 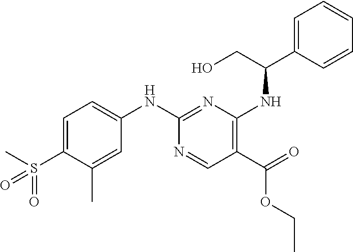 [SMILES: CCOC(=O)c1cnc(Nc2ccc(S(C)(=O)=O)c(C)c2)nc1N[C@@H](CO)c1ccccc1] | ethyl 4-[[(1R)-2-hydroxy-1-phenyl- ethyl]amino]-2-(3-methyl-4- methylsulfonyl-anilino)pyrimidine-5- carboxylate | 471.2 | 471 |
| 170 | 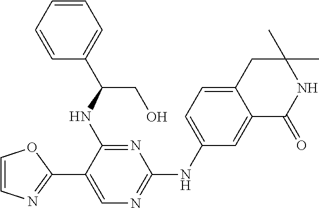 [SMILES: CC1(C)Cc2ccc(Nc3ncc(-c4ncco4)c(N[C@H](CO)c4ccccc4)n3)cc2C(=O)N1] | 7-[[4-[[(1S)-2-hydroxy-1-phenyl- ethyl]amino]-5-oxazol-2-yl- pyrimidin-2-yl]amino]-3,3-dimethyl- 2,4-dihydroisoquinolin-1-one | 471.2 | 471 |
| 171 | 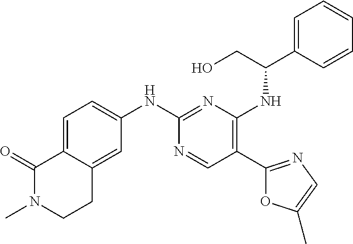 [SMILES: Cc1cnc(-c2cnc(Nc3ccc4c(c3)CCN(C)C4=O)nc2N[C@H](CO)c2ccccc2)o1] | 6-[[4-[[(1S)-2-hydroxy-1-phenyl- ethyl]amino]-5-(5-methyloxazol-2- yl)pyrimidin-2-yl]amino]-2-methyl- 3,4-dihydroisoquinolin-1-one | 471.2 | 471 |
| 172 | 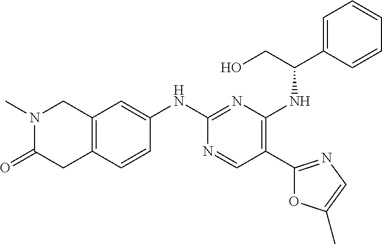 [SMILES: Cc1cnc(-c2cnc(Nc3ccc4c(c3)CN(C)C(=O)C4)nc2N[C@H](CO)c2ccccc2)o1] | 7-[[4-[[(1S)-2-hydroxy-1-phenyl- ethyl]amino]-5-(5-methyloxazol-2- yl)pyrimidin-2-yl]amino]-2-methyl- 1,4-dihydroisoquinolin-3-one | 471.2 | 471 |
| 173 | 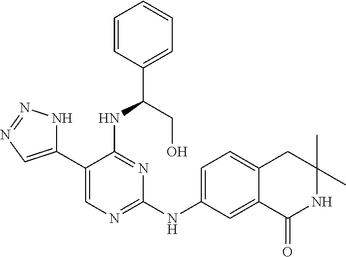 [SMILES: CC1(C)Cc2ccc(Nc3ncc(-c4cnn[nH]4)c(N[C@H](CO)c4ccccc4)n3)cc2C(=O)N1] | 7-[[4-[[(1S)-2-hydroxy-1-phenyl- ethyl]amino]-5-(1H-triazol-5- yl)pyrimidin-2-yl]amino]-3,3- dimethyl-2,4-dihydroisoquinolin-1- one | 471.2 | 471 |
| 174 | 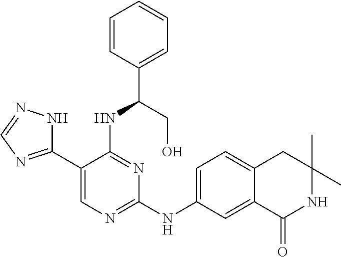 [SMILES: CC1(C)Cc2ccc(Nc3ncc(-c4ncn[nH]4)c(N[C@H](CO)c4ccccc4)n3)cc2C(=O)N1] | 7-[[4-[[(1S)-2-hydroxy-1-phenyl- ethyl]amino]-5-(1H-1,2,4-triazol-5- yl)pyrimidin-2-yl]amino]-3,3- dimethyl-2,4-dihydroisoquinolin-1- one | 471.2 | 471 |
| 175 | 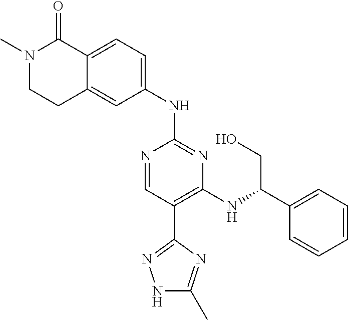 [SMILES: Cc1nc(-c2cnc(Nc3ccc4c(c3)CCN(C)C4=O)nc2N[C@H](CO)c2ccccc2)n[nH]1] | 6-[[4-[[(1S)-2-hydroxy-1-phenyl- ethyl]amino]-5-(5-methyl-1H-1,2,4- triazol-3-yl)pyrimidin-2-yl)amino]-2- methyl-3,4-dihydrisoqouinolin-1-one | 471.2 | 471 |
| 176 | 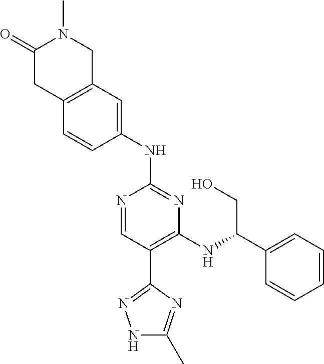 [SMILES: Cc1nc(-c2cnc(Nc3ccc4c(c3)CN(C)C(=O)C4)nc2N[C@H](CO)c2ccccc2)n[nH]1] | 7-[[4-[[(1S)-2-hydroxy-1-phenyl- ethyl]amino]-5-(5-methyl-1H-1,2,4- triazol-3-yl)pyrimidin-2-yl]amino]-2- methyl-1,4-dihydroisoquinolin-3-one | 471.2 | 471 |
| 177 | 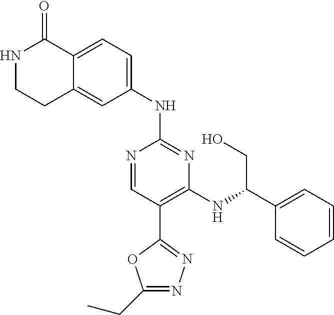 [SMILES: CCc1nnc(-c2cnc(Nc3ccc4c(c3)CCNC4=O)nc2N[C@H](CO)c2ccccc2)o1] | 6-[[5-(5-ethyl-1,3,4-oxadiazol-2-yl)- 4-[[(1S)-2-hydroxy-1-phenyl- ethyl]amino]pyrimidin-2-yl]amino]- 3,4-dihydro-2H-isoquinolin-1-one | 472.2 | 472 |
| 178 | 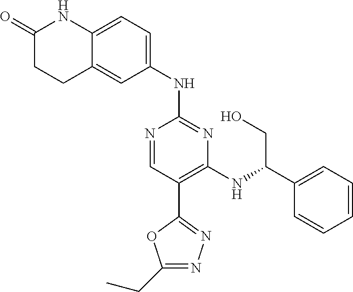 [SMILES: CCc1nnc(-c2cnc(Nc3ccc4c(c3)CCC(=O)N4)nc2N[C@H](CO)c2ccccc2)o1] | 6-[[5-(5-ethyl-1,3,4-oxadiazol-2-yl)- 4-[[(1S)-2-hydroxy-1-phenyl- ethyl]amino]pyrimidin-2-yl]amino]- 3,4-dihydro-1H-quinolin-2-one | 472.2 | 472 |
| 179 | 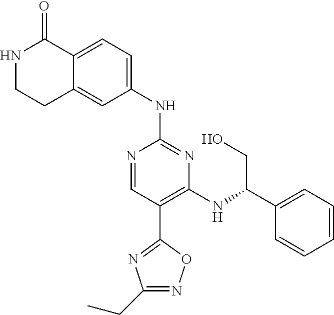 [SMILES: CCc1noc(-c2cnc(Nc3ccc4c(c3)CCNC4=O)nc2N[C@H](CO)c2ccccc2)n1] | 6-[[5-(3-ethyl-1,2,4-oxadiazol-5-yl)- 4-[[(1S)-2-hydroxy-1-phenyl- ethyl]amino]pyrimidin-2-yl]amino]- 3,4-dihydro-2H-isoquinolin-1-one | 472.2 | 472 |
| 180 | 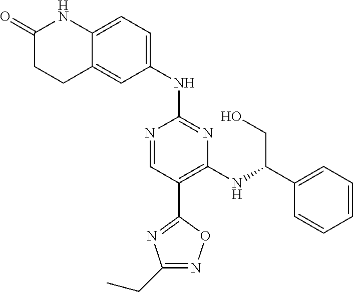 [SMILES: CCc1noc(-c2cnc(Nc3ccc4c(c3)CCC(=O)N4)nc2N[C@H](CO)c2ccccc2)n1] | 6-[[5-(3-ethyl-1,2,4-oxadiazol-5-yl)- 4-[[(1S)-2-hydroxy-1-phenyl- ethyl]amino]pyrimidin-2-yl]amino]- 3,4-dihydro-1H-quinolin-2-one | 472.2 | 472 |
| 181 | 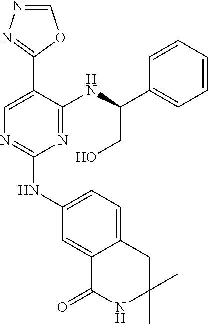 [SMILES: CC1(C)Cc2ccc(Nc3ncc(-c4nnco4)c(N[C@H](CO)c4ccccc4)n3)cc2C(=O)N1] | 7-[[4-[[(1S)-2-hydroxy-1-phenyl- ethyl]amino]-5-(1,3,4-oxadiazol-2- yl)pyrimidin-2-yl]amino]-3,3- dimethyl-2,4-dihydroisoquinolin-1- one | 472.2 | 472 |
| 182 | 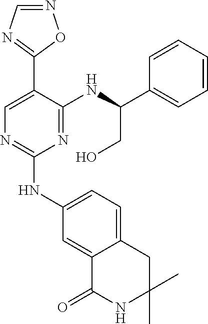 [SMILES: CC1(C)Cc2ccc(Nc3ncc(-c4ncno4)c(N[C@H](CO)c4ccccc4)n3)cc2C(=O)N1] | 7-[[4-[[(1S)-2-hydroxy-1-phenyl- ethyl]amino]-5-(1,2,4-oxadiazol-5- yl)pyrimidin-2-yl]amino]-3,3- dimethyl-2,4-dihydroisoquinolin-1- one | 472.2 | 472 |
| 183 | 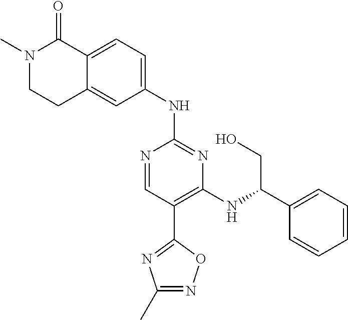 [SMILES: Cc1noc(-c2cnc(Nc3ccc4c(c3)CCN(C)C4=O)nc2N[C@H](CO)c2ccccc2)n1] | 6-[[4-[[(1S)-2-hydroxy-1-phenyl- ethyl]amino]-5-(3-methyl-1,2,4- oxadiazol-5-yl)pyrimidin-2- yl]amino]-2-methyl-3,4- dihydroisoquinolin-1-one | 472.2 | 472 |
| 184 | 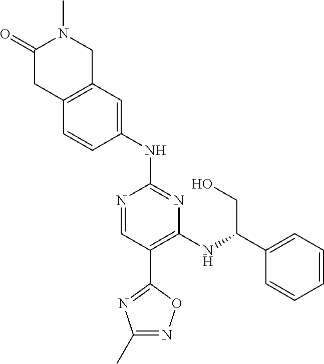 [SMILES: Cc1noc(-c2cnc(Nc3ccc4c(c3)CN(C)C(=O)C4)nc2N[C@H](CO)c2ccccc2)n1] | 7-[[4-[[(1S)-2-hydroxy-1-phenyl- ethyl]amino]-5-(3-methyl-1,2,4- oxadiazol-5-yl)pyrimidin-2- yl]amino]-2-methyl-1,4- dihydroisoquinolin-3-one | 472.2 | 472 |
| 185 | 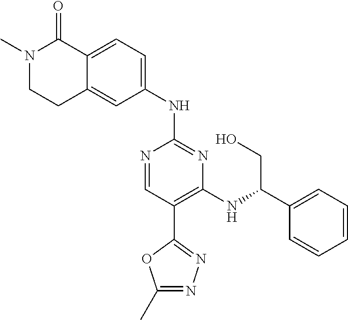 [SMILES: Cc1nnc(-c2cnc(Nc3ccc4c(c3)CCN(C)C4=O)nc2N[C@H](CO)c2ccccc2)o1] | 6-[[4-[[(1S)-2-hydroxy-1-phenyl- ethyl]amino]-5-(5-methyl-1,3,4- oxadiazol-2-yl)pyrimidin-2- yl]amino]-2-methyl-3,4- dihydroisoquinolin-1-one | 472.2 | 472 |
| 186 | 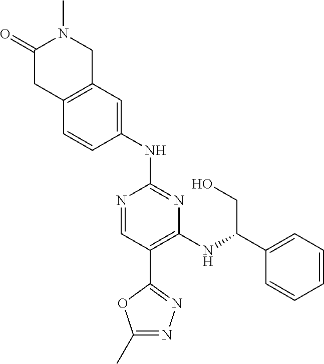 [SMILES: Cc1nnc(-c2cnc(Nc3ccc4c(c3)CN(C)C(=O)C4)nc2N[C@H](CO)c2ccccc2)o1] | 7-[[4-[[(1S)-2-hydroxy-1-phenyl- ethyl]amino]-5-(5-methyl-1,3,4- oxadiazol-2-yl)pyrimidin-2- yl]amino]-2-methyl-1,4- dihydroisoquinolin-3-one | 472.2 | 472 |
| 187 | 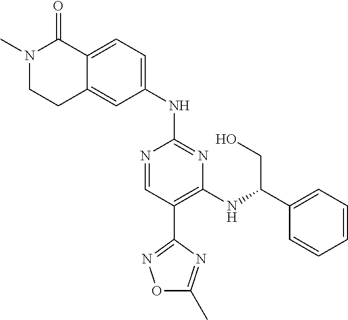 [SMILES: Cc1nc(-c2cnc(Nc3ccc4c(c3)CCN(C)C4=O)nc2N[C@H](CO)c2ccccc2)no1] | 6-[[4-[[(1S)-2-hydroxy-1-phenyl- ethyl]amino]-5-(5-methyl-1,2,4- oxadiazol-3-yl)pyrimidin-2- yl]amino]-2-methyl-3,4- dihydroisoquinolin-1-one | 472.2 | 472 |
| 188 | 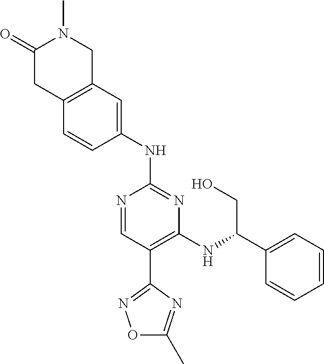 [SMILES: Cc1nc(-c2cnc(Nc3ccc4c(c3)CN(C)C(=O)C4)nc2N[C@H](CO)c2ccccc2)no1] | 7-[[4-[[(1S)-2-hydroxy-1-phenyl- ethyl]amino]-5-(5-methyl-1,2,4- oxadiazol-3-yl)pyrimidin-2- yl]amino]-2-methyl-1,4- dihydroisoquinolin-3-one | 472.2 | 472 |
| 189 | 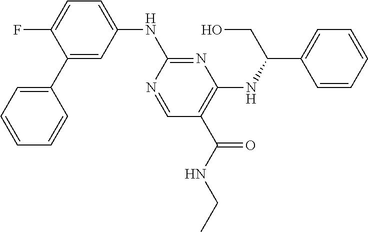 [SMILES: CCNC(=O)c1cnc(Nc2ccc(F)c(-c3ccccc3)c2)nc1N[C@H](CO)c1ccccc1] | N-ethyl-2-(4-fluoro-3-phenyl- anilino)-4-[[(1S)-2-hydroxy-1-phenyl- ethyl]amino]pyrimidine-5- carboxamide | 472.2 | 472 |
| 190 | 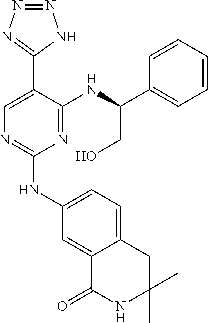 [SMILES: CC1(C)Cc2ccc(Nc3ncc(-c4nnn[nH]4)c(N[C@H](CO)c4ccccc4)n3)cc2C(=O)N1] | 7-[[4-[[(1S)-2-hydroxy-1-phenyl- ethyl]amino]-5-(1H-tetrazol-5- yl)pyrimidin-2-yl]amino]-3,3- dimethyl-2,4-dihydroisoquinolin-1- one | 472.2 | 472 |
| 191 | 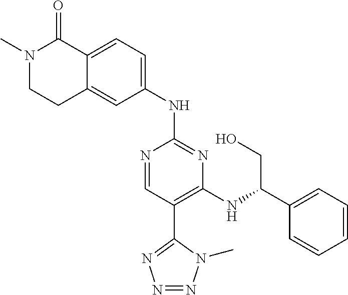 [SMILES: CN1CCc2cc(Nc3ncc(-c4nnnn4C)c(N[C@H](CO)c4ccccc4)n3)ccc2C1=O] | 6-[[4-[[(1S)-2-hydroxy-1-phenyl- ethyl]amino]-5-(1-methyltetrazol-5- yl)pyrimidin-2-yl]amino]-2-methyl- 3,4-dihydroisoquinolin-1-one | 472.2 | 472 |
| 192 | 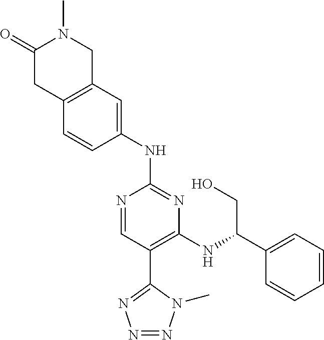 [SMILES: CN1Cc2cc(Nc3ncc(-c4nnnn4C)c(N[C@H](CO)c4ccccc4)n3)ccc2CC1=O] | 7-[[4-[[(1S)-2-hydroxy-1-phenyl- ethyl]amino]-5-(1-methyltetrazol-5- yl)pyrimidin-2-yl]amino]-2-methyl- 1,4-dihydroisoquinolin-3-one | 472.2 | 472 |
| 193 | 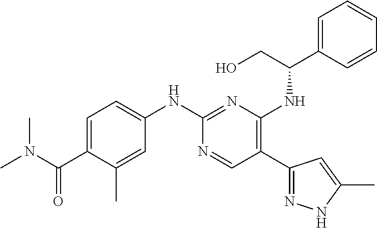 [SMILES: Cc1cc(-c2cnc(Nc3ccc(C(=O)N(C)C)c(C)c3)nc2N[C@H](CO)c2ccccc2)n[nH]1] | 4-[[4-[[(1S)-2-hydroxy-1-phenyl- ethyl]amino]-5-(5-methyl-1H- pyrazol-3-yl)pyrimidin-2-yl]amino]- N,N,2-trimethyl-benzamide | 472.2 | 472 |
| 194 | 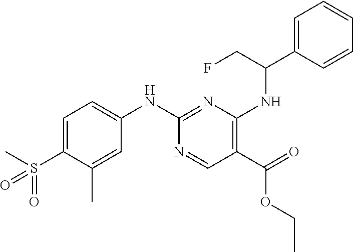 [SMILES: CCOC(=O)c1cnc(Nc2ccc(S(C)(=O)=O)c(C)c2)nc1NC(CF)c1ccccc1] | ethyl 4-[[2-fluoro-1-phenyl- ethyl)amino]-2-(3-methyl-4- methylsulfonyl-anilino)pyrimidine-5- carboxylate | 473.2 | 473 |
| 195 | 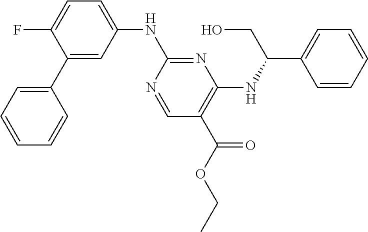 [SMILES: CCOC(=O)c1cnc(Nc2ccc(F)c(-c3ccccc3)c2)nc1N[C@H](CO)c1ccccc1] | ethyl 2-(4-fluoro-3-phenyl-anilino)-4- [[(1S)-2-hydroxy-1-phenyl- ethyl]amino]pyrimidine-5-carboxylate | 473.2 | 473 |
| 196 | 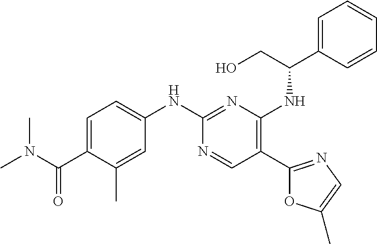 [SMILES: Cc1cnc(-c2cnc(Nc3ccc(C(=O)N(C)C)c(C)c3)nc2N[C@H](CO)c2ccccc2)o1] | 4-[[4-[[(1S)-2-hydroxy-1-phenyl- ethyl]amino]-5-(5-methyloxazol-2- yl)pyrimidin-2-yl]amino]-N,N,2- trimethyl-benzamide | 473.2 | 473 |
| 197 | 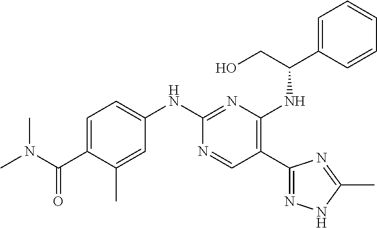 [SMILES: Cc1nc(-c2cnc(Nc3ccc(C(=O)N(C)C)c(C)c3)nc2N[C@H](CO)c2ccccc2)n[nH]1] | 4-[[4-[[(1S)-2-hydroxy-1-phenyl- ethyl]amino]-5-(5-methyl-1H-1,2,4- triazol-3-yl)pyrimidin-2-yl]amino]- N,N,2-trimethyl-benzamide | 473.2 | 473 |
| 198 | 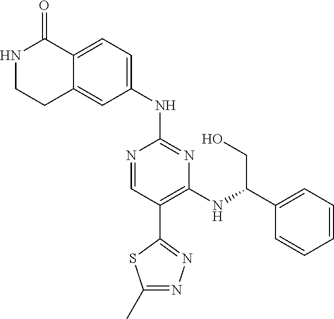 [SMILES: Cc1nnc(-c2cnc(Nc3ccc4c(c3)CCNC4=O)nc2N[C@H](CO)c2ccccc2)s1] | 6-[[4-[[(1S)-2-hydroxy-1-phenyl- ethyl]amino]-5-(5-methyl-1,3,4- thiadiazol-2-yl)pyrimidin-2- yl]amino]-3,4-dihydro-2H- isoquinolin-1-one | 474.2 | 474 |
| 199 | 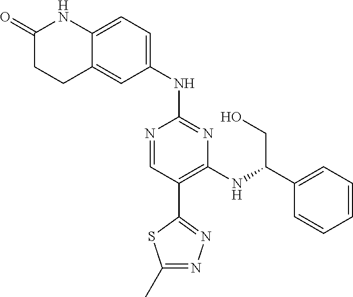 [SMILES: Cc1nnc(-c2cnc(Nc3ccc4c(c3)CCC(=O)N4)nc2N[C@H](CO)c2ccccc2)s1] | 6-[[4-[[(1S)-2-hydroxy-1-phenyl- ethyl]amino]-5-(5-methyl-1,3,4- thiadiazol-2-yl]pyrimidin-2- yl]amino]-3,4-dihydro-1H-quinolin-2- one | 474.2 | 474 |
| 200 | 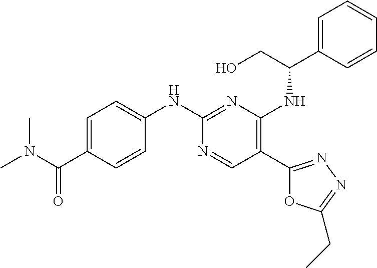 [SMILES: CCc1nnc(-c2cnc(Nc3ccc(C(=O)N(C)C)cc3)nc2N[C@H](CO)c2ccccc2)o1] | 4-[[5-(5-ethyl-1,3,4-oxadiazol-2-yl)- 4-[[(1S)-2-hydroxy-1-phenyl- ethyl]amino]pyrimidin-2-yl]amino]- N,N-dimethyl-benzamide | 474.2 | 474 |
| 201 | 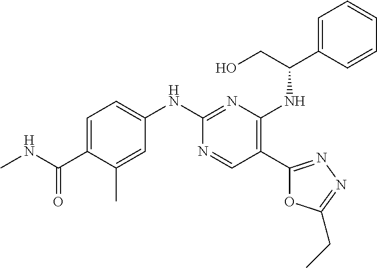 [SMILES: CCc1nnc(-c2cnc(Nc3ccc(C(=O)NC)c(C)c3)nc2N[C@H](CO)c2ccccc2)o1] | 4-[[5-(5-ethyl-1,3,4-oxadiazol-2-yl)- 4-[[(1S)-2-hydroxy-1-phenyl- ethyl]amino]pyrimidin-2-yl]amino]- N,2-dimethyl-benzamide | 474.2 | 474 |
| 202 | 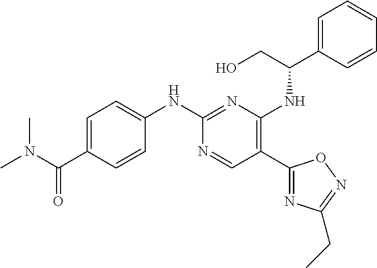 [SMILES: CCc1noc(-c2cnc(Nc3ccc(C(=O)N(C)C)cc3)nc2N[C@H](CO)c2ccccc2)n1] | 4-[[5-(3-ethyl-1,2,4-oxadiazol-5-yl)- 4-[[(1S)-2-hydroxy-1-phenyl- ethyl]amino]pyrimidin-2-yl]amino]- N,N-dimethyl-benzamide | 474.2 | 474 |
| 203 | 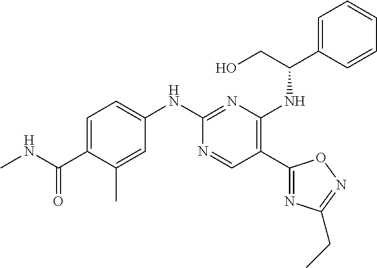 [SMILES: CCc1noc(-c2cnc(Nc3ccc(C(=O)NC)c(C)c3)nc2N[C@H](CO)c2ccccc2)n1] | 4-[[5-(3-ethyl-1,2,4-oxadiazol-5-yl)- 4-[[(1S)-2-hydroxy-1-phenyl- ethyl]amino]pyrimidin-2-yl]amino]- N,2-dimethyl-benzamide | 474.2 | 474 |
| 204 | 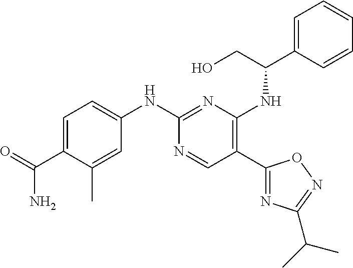 [SMILES: Cc1cc(Nc2ncc(-c3nc(C(C)C)no3)c(N[C@H](CO)c3ccccc3)n2)ccc1C(N)=O] | 4-[[4-[[(1S)-2-hydroxy-1-phenyl- ethyl]amino]-5-(3-isopropyl-1,2,4- oxadiazol-5-yl)pyrimidin-2- yl]amino]-2-methyl-benzamide | 474.2 | 474 |
| 205 | 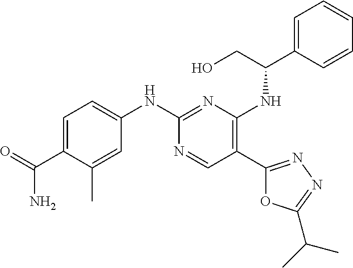 [SMILES: Cc1cc(Nc2ncc(-c3nnc(C(C)C)o3)c(N[C@H](CO)c3ccccc3)n2)ccc1C(N)=O] | 4-[[4-[[(1S)-2-hydroxy-1-phenyl- ethyl]amino]-5-(5-isopropyl-1,3,4- oxadiazol-2-yl)pyrimidin-2- yl]amino]-2-methyl-benzamide | 474.2 | 474 |
| 206 | 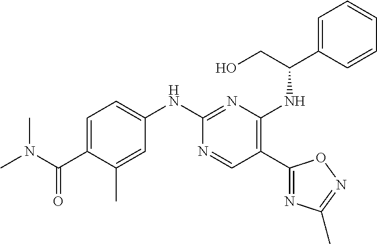 [SMILES: Cc1noc(-c2cnc(Nc3ccc(C(=O)N(C)C)c(C)c3)nc2N[C@H](CO)c2ccccc2)n1] | 4-[[4-[[(1S)-2-hydroxy-1-phenyl- ethyl]amino]-5-(3-methyl-1,2,4- oxadiazol-5-yl)pyrimidin-2- yl]amino]-N,N,2-trimethyl-benzamide | 474.2 | 474 |
| 207 | 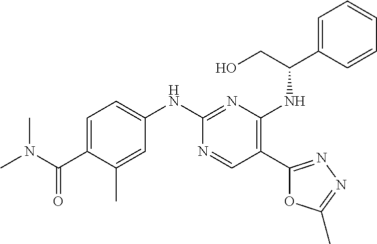 [SMILES: Cc1nnc(-c2cnc(Nc3ccc(C(=O)N(C)C)c(C)c3)nc2N[C@H](CO)c2ccccc2)o1] | 4-[[4-[[(1S)-2-hydroxy-1-phenyl- ethyl]amino]-5-(5-methyl-1,3,4- oxadiazol-2-yl)pyrimidin-2- yl]amino]-N,N,2-trimethyl-benzamide | 474.2 | 474 |
| 208 | 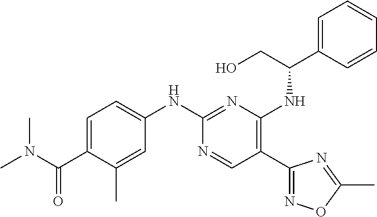 [SMILES: Cc1nc(-c2cnc(Nc3ccc(C(=O)N(C)C)c(C)c3)nc2N[C@H](CO)c2ccccc2)no1] | 4-[[4-[[(1S)-2-hydroxy-1-phenyl- ethyl]amino]-5-(5-methyl-1,2,4- oxadiazol-3-yl)pyrimidin-2- yl]amino]-N,N,2-trimethyl-benzamide | 474.2 | 474 |
| 209 | 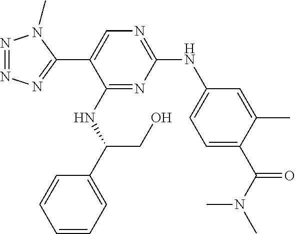 [SMILES: Cc1cc(Nc2ncc(-c3nnnn3C)c(N[C@H](CO)c3ccccc3)n2)ccc1C(=O)N(C)C] | 4-[[4-[[(1S)-2-hydroxy-1-phenyl- ethyl]amino]-5-(1-methyltetrazol-5- yl)pyrimidin-2-yl]amino]-N,N,2- trimethyl-benzamide | 474.2 | 474 |
| 210 | 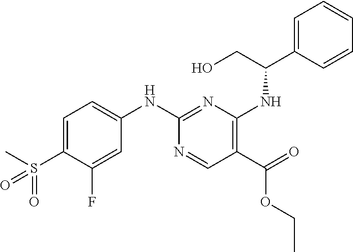 [SMILES: CCOC(=O)c1cnc(Nc2ccc(S(C)(=O)=O)c(F)c2)nc1N[C@H](CO)c1ccccc1] | ethyl 2-(3-fluoro-4-methylsulfonyl- anilino)-4-[[(1S)-2-hydroxy-1-phenyl- ethyl]amino]-pyrimidine-5- carboxylate | 475.1 | 475 |
| 211 | 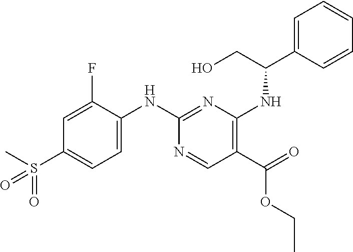 [SMILES: CCOC(=O)c1cnc(Nc2ccc(S(C)(=O)=O)cc2F)nc1N[C@H](CO)c1ccccc1] | ethyl 2-(2-fluoro-4-methylsulfonyl- anilino)-4-[[(1S)-2-hydroxy-1-phenyl- ethyl]amino]pyrimidine-5-carboxylate | 475.1 | 475 |
| 212 | 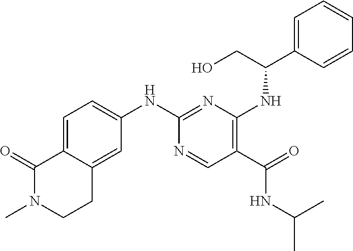 [SMILES: CC(C)NC(=O)c1cnc(Nc2ccc3c(c2)CCN(C)C3=O)nc1N[C@H](CO)c1ccccc1] | 4-[[(1S)-2-hydroxy-1-phenyl- ethyl]amino]-N-isopropyl-2-[(2- methyl-1-oxo-3,4-dihydroisoquinolin- 6-yl)amino)pyrimidine-5- carboxamide | 475.2 | 475 |
| 213 | 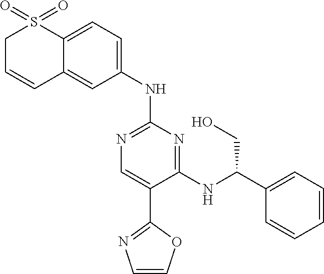 [SMILES: O=S1(=O)CC=Cc2cc(Nc3ncc(-c4ncco4)c(N[C@H](CO)c4ccccc4)n3)ccc21] | (2S)-2-[[2-[(1,1-dioxo-2H- thiochromen-6-yl)amino]-5-oxazol-2- yl-pyrimidin-4-yl]amino]-2-phenyl- ethanol | 476.1 | 476 |
| 214 | 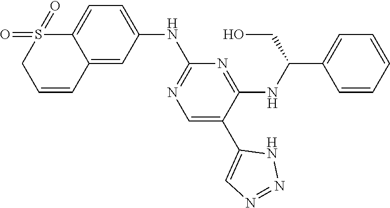 [SMILES: O=S1(=O)CC=Cc2cc(Nc3ncc(-c4cnn[nH]4)c(N[C@H](CO)c4ccccc4)n3)ccc21] | (2S)-2-[[2-[(1,1-dioxo-2H- thiochromen-6-yl)amino]-5-(1H- triazol-5-yl)pyrimidin-4-yl]amino]-2- phenyl-ethanol | 476.2 | 476 |
| 215 | 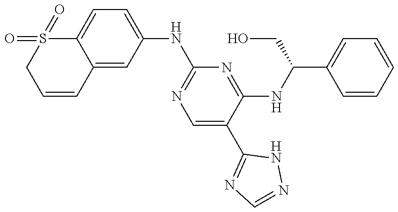 [SMILES: O=S1(=O)CC=Cc2cc(Nc3ncc(-c4ncn[nH]4)c(N[C@H](CO)c4ccccc4)n3)ccc21] | (2S)-2-[[2-[(1,1-dioxo-2H- thiochromen-6-yl)amino]-5-(1H- 1,2,4-triazol-5-yl)pyrimidin-4- yl]amino]-2-phenyl-ethanol | 476.2 | 476 |
| 216 | 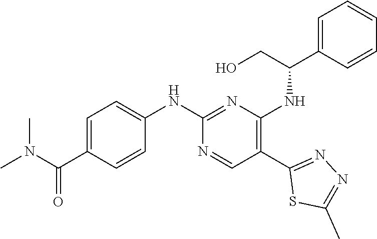 [SMILES: Cc1nnc(-c2cnc(Nc3ccc(C(=O)N(C)C)cc3)nc2N[C@H](CO)c2ccccc2)s1] | 4-[[4-[[(1S)-2-hydroxy-1-phenyl- ethyl]amino]-5-(5-methyl-1,3,4- thiadiazol-2-yl)pyrimidin-2- yl]amino]-N,N-dimethyl-benzamide | 476.2 | 476 |
| 217 | 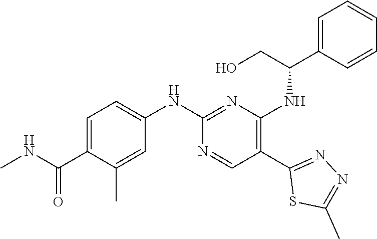 [SMILES: CNC(=O)c1ccc(Nc2ncc(-c3nnc(C)s3)c(N[C@H](CO)c3ccccc3)n2)cc1C] | 4-[[4-[[(1S)-2-hydroxy-1-phenyl- ethyl]amino]-5-(5-methyl-1,3,4- thiadiazol-2-yl)pyrimidin-2- yl]amino]-N,2-dimethyl-benzamide | 476.2 | 476 |
| 218 | 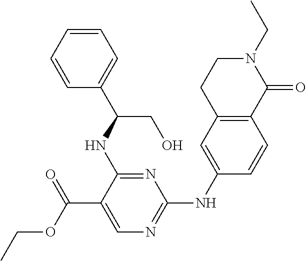 [SMILES: CCOC(=O)c1cnc(Nc2ccc3c(c2)CCN(CC)C3=O)nc1N[C@H](CO)c1ccccc1] | ethyl 2-[(2-ethyl-1-oxo-3,4- dihydroisoquinolin-6-yl)amino]-4- [[(1S)-2-hydroxy-1-phenyl- ethyl]amino]pyrimidine-5-carboxylate | 476.2 | 476 |
| 219 | 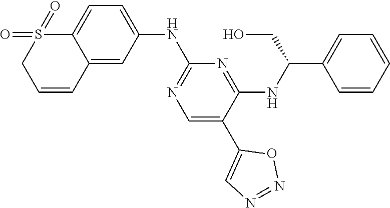 [SMILES: O=S1(=O)CC=Cc2cc(Nc3ncc(-c4cnno4)c(N[C@H](CO)c4ccccc4)n3)ccc21] | (2S)-2-[[2-[(1,1-dioxo-2H- thiochromen-6-yl)amino]-5-(1,2,4- oxadiazol-5-yl)pyrimidin-4- yl]amino]-2-phenyl-ethanol | 477.1 | 477 |
| 220 | 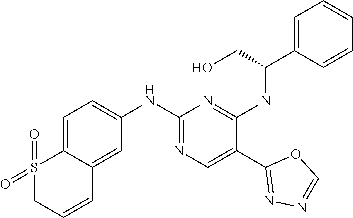 | (2S)-2-[[2-[(1,1-dioxo-2H- thiochromen-6-yl)amino]-5-(1,3,4- oxadiazol-2-yl)pyrimidin-4- yl]amino]-2-phenyl-ethanol | 477.1 | 477 |
| 221 | 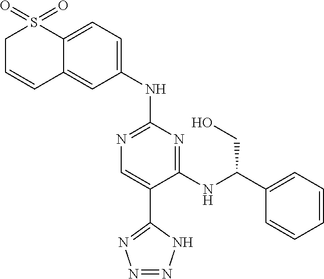 [SMILES: O=S1(=O)CC=Cc2cc(Nc3ncc(-c4nnn[nH]4)c(N[C@H](CO)c4ccccc4)n3)ccc21] | (2S)-2-[[2-[(1,1-dioxo-2H- thiochromen-6-yl)amino]-5-(1H- tetrazol-5-yl)pyrimidin-4-yl]amino]- 2-phenyl-ethanol | 477.1 | 477 |
| 222 | 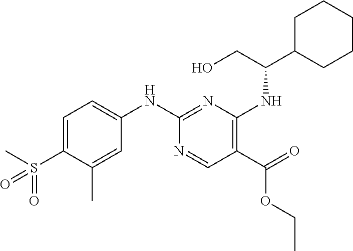 [SMILES: CCOC(=O)c1cnc(Nc2ccc(S(C)(=O)=O)c(C)c2)nc1N[C@H](CO)C1CCCCC1] | ethyl 4-[[(1S)-1-cyclohexyl-2- hydroxyethyl]amino]-2-(3-methyl-4- methylsulfonyl-anilino)pyrimidine-5- carboxylate | 477.2 | 477 |
| 223 | 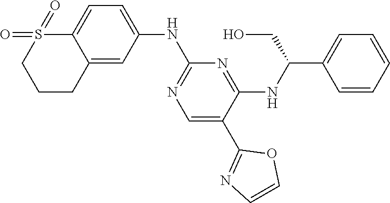 [SMILES: O=S1(=O)CCCc2cc(Nc3ncc(-c4ncco4)c(N[C@H](CO)c4ccccc4)n3)ccc21] | (2S)-2-[[2-[(1,1-dioxo-3,4-dihydro- 2H-thiochromen-6-yl)amino]-5- oxazol-2-yl-pyrimidin-4-yl]amino]-2- phenyl-ethanol | 478.2 | 478 |
| 224 | 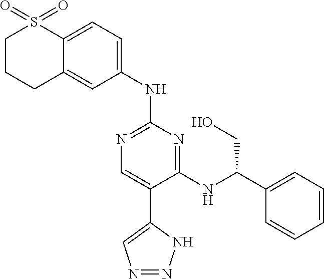 [SMILES: O=S1(=O)CCCc2cc(Nc3ncc(-c4cnn[nH]4)c(N[C@H](CO)c4ccccc4)n3)ccc21] | (2S)-2-[[2-[(1,1-dioxo-3,4-dihydro- 2H-thiochromen-6-yl)amino]-5-(1H- triazol-5-yl)pyrimidin-4-yl]amino]-2- phenyl-ethanol | 478.2 | 478 |
| 225 | 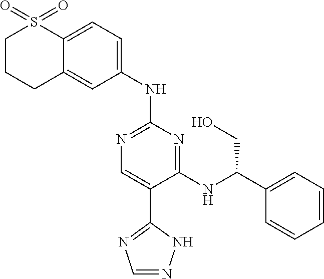 [SMILES: O=S1(=O)CCCc2cc(Nc3ncc(-c4ncn[nH]4)c(N[C@H](CO)c4ccccc4)n3)ccc21] | (2S)-2-[[2-[(1,1-dioxo-3,4-dihydro- 2H-thiochromen-6-yl)amino]-5-(1H- 1,2,4-triazol-5-yl)pyrimidin-4- yl]amino]-2-phenyl-ethanol | 478.2 | 478 |
| 226 | 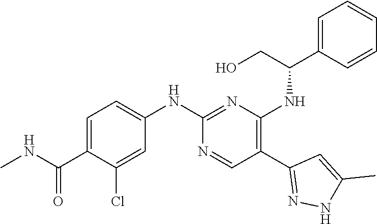 [SMILES: CNC(=O)c1ccc(Nc2ncc(-c3cc(C)[nH]n3)c(N[C@H](CO)c3ccccc3)n2)cc1Cl] | 2-chloro-4-[[4-[[(1S)-2-hydroxy-1- phenyl-ethyl]amino]-5-(5-methyl-1H- pyrazol-3-yl)pyrimidin-2-yl]amino]- N-methyl-benzamide | 478.2 | 478 |
| 227 | 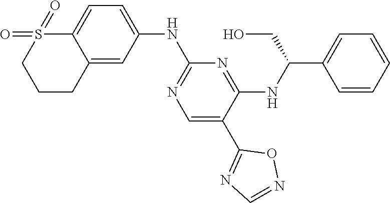 [SMILES: O=S1(=O)CCCc2cc(Nc3ncc(-c4ncno4)c(N[C@H](CO)c4ccccc4)n3)ccc21] | (2S)-2-[[2-[(1,1-dioxo-3,4-dihydro- 2H-thiochromen-6-yl)amino]-5- (1,2,4-oxadiazol-5-yl)pyrimidin-4- yl]amino]-2-phenyl-ethanol | 479.2 | 479 |
| 228 | 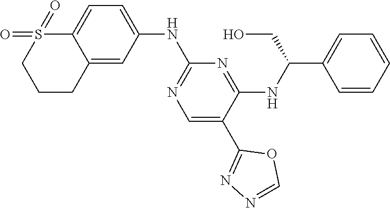 [SMILES: O=S1(=O)CCCc2cc(Nc3ncc(-c4nnco4)c(N[C@H](CO)c4ccccc4)n3)ccc21] | (2S)-2-[[2-[(1,1-dioxo-3,4-dihydro- 2H-thiochromen-6-yl)amino]-5- (1,3,4-oxadiazol-2-yl)pyrimidin-4- yl]amino]-2-phenyl-ethanol | 479.2 | 479 |
| 229 | 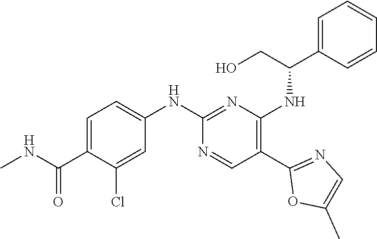 [SMILES: CNC(=O)c1ccc(Nc2ncc(-c3ncc(C)o3)c(N[C@H](CO)c3ccccc3)n2)cc1Cl] | 2-chloro-4-[[4-[[(1S)-2-hydroxy-1- phenyl-ethyl]amino]-5-(5- methyloxazol-2-yl)pyrimidin-2- yl]amino]-N-methyl-benzamide | 479.2 | 479 |
| 230 | 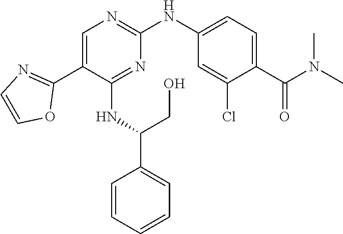 [SMILES: CN(C)C(=O)c1ccc(Nc2ncc(-c3ncco3)c(N[C@H](CO)c3ccccc3)n2)cc1Cl] | 2-chloro-4-[[4-[[(1S)-2-hydroxy-1- phenyl-ethyl]amino]-5-oxazol-2-yl- pyrimidin-2-yl]amino]-N,N-dimethyl- benzamide | 479.2 | 479 |
| 231 | 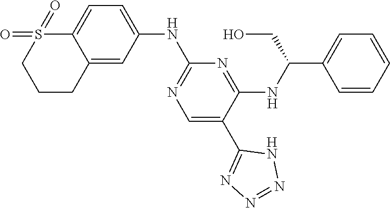 [SMILES: O=S1(=O)CCCc2cc(Nc3ncc(-c4nnn[nH]4)c(N[C@H](CO)c4ccccc4)n3)ccc21] | (2S)-2-[[2-[(1,1-dioxo-3,4-dihydro- 2H-thiochromen-6-yl)amino]-5-(1H- tetrazol-5-yl)pyrimidin-4-yl]amino]- 2-phenyl-ethanol | 479.2 | 479 |
| 232 | 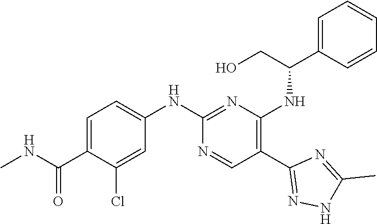 [SMILES: CNC(=O)c1ccc(Nc2ncc(-c3n[nH]c(C)n3)c(N[C@H](CO)c3ccccc3)n2)cc1Cl] | 2-chloro-4-[[4-[[(1S)-2-hydroxy-1- phenyl-ethyl]amino]-5-(5-methyl-1H- 1,2,4-triazol-3-yl)pyrimidin-2- yl]amino]-N-methyl-benzamide | 479.2 | 479 |
| 233 | 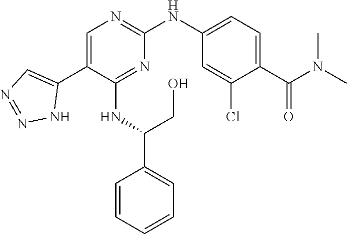 [SMILES: CN(C)C(=O)c1ccc(Nc2ncc(-c3cnn[nH]3)c(N[C@H](CO)c3ccccc3)n2)cc1Cl] | 2-chloro-4-[[4-[[(1S)-2-hydroxy-1- phenyl-ethyl]amino]-5-(1H-triazol-5- yl)pyrimidin-2-yl]amino]-N,N- dimethyl-benzamide | 479.2 | 479 |
| 234 | 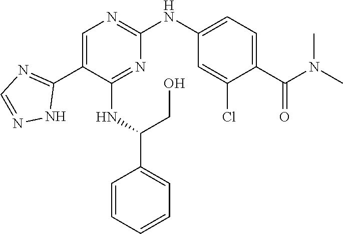 [SMILES: CN(C)C(=O)c1ccc(Nc2ncc(-c3ncn[nH]3)c(N[C@H](CO)c3ccccc3)n2)cc1Cl] | 2-chloro-4-[[4-[[(1S)-2-hydroxy-1- phenyl-ethyl]amino]-5-(1H-1,2,4- triazol-5-yl)pyrimidin-2-yl]amino]- N,N-dimethyl-benzamide | 479.2 | 479 |
| 235 | 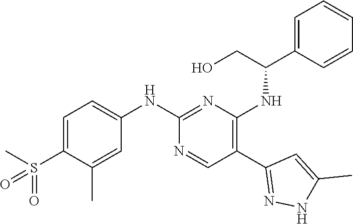 [SMILES: Cc1cc(-c2cnc(Nc3ccc(S(C)(=O)=O)c(C)c3)nc2N[C@H](CO)c2ccccc2)n[nH]1] | (2S)-2-[[2-(3-methyl-4- methylsulfonyl-anilino)-5-(5-methyl- 1H-pyrazol-3-yl)pyrimidin-4- yl]amino]-2-phenyl-ethanol | 479.2 | 479 |
| 236 | 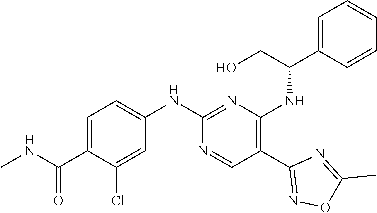 [SMILES: CNC(=O)c1ccc(Nc2ncc(-c3noc(C)n3)c(N[C@H](CO)c3ccccc3)n2)cc1Cl] | 2-chloro-4-[[4-[[(1S)-2-hydroxy-1- phenyl-ethyl]amino]-5-(5-methyl- 1,2,4-oxadiazol-3-yl)pyrimidin-2- yl]amino]-N-methyl-benzamide | 480.2 | 480 |
| 237 | 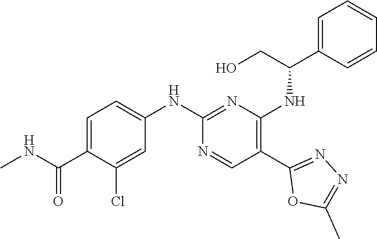 [SMILES: CNC(=O)c1ccc(Nc2ncc(-c3nnc(C)o3)c(N[C@H](CO)c3ccccc3)n2)cc1Cl] | 2-chloro-4-[[4-[[(1S)-2-hydroxy-1- phenyl-ethyl]amino]-5-(5-methyl- 1,3,4-oxadiazol-2-yl)pyrimidin-2- yl]amino]-N-methyl-benzamide | 480.2 | 480 |
| 238 | 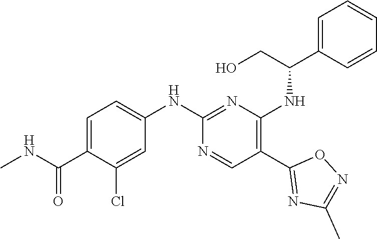 [SMILES: CNC(=O)c1ccc(Nc2ncc(-c3nc(C)no3)c(N[C@H](CO)c3ccccc3)n2)cc1Cl] | 2-chloro-4-[[4-[[(1S)-2-hydroxy-1- phenyl-ethyl]amino]-5-(3-methyl- 1,2,4-oxadiazol-5-yl)pyrimidin-2 yl]amino]-N-methyl-benzamide | 480.2 | 480 |
| 239 | 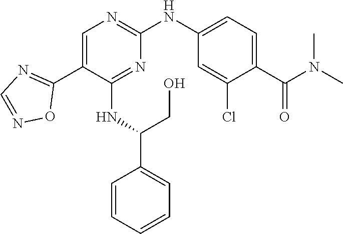 [SMILES: CN(C)C(=O)c1ccc(Nc2ncc(-c3ncno3)c(N[C@H](CO)c3ccccc3)n2)cc1Cl] | 2-chloro-4-[[4-[[(1S)-2-hydroxy-1- phenyl-ethyl]amino]-5-(1,2,4- oxadiazol-5-yl)pyrimidin-2- yl]amino]-N,N-dimethyl-benzamide | 480.2 | 480 |
| 240 | 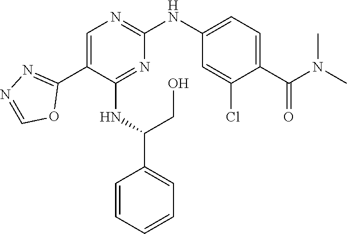 [SMILES: CN(C)C(=O)c1ccc(Nc2ncc(-c3nnco3)c(N[C@H](CO)c3ccccc3)n2)cc1Cl] | 2-chloro-4-[[4-[[(1S)-2-hydroxy-1- phenyl-ethyl]amino]-5-(1,3,4- oxadiazol-2-yl)pyrimidin-2- yl]amino]-N,N-dimethyl-benzamide | 480.2 | 480 |
| 241 | 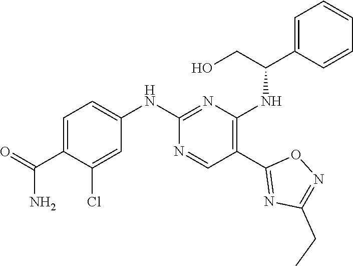 [SMILES: CCc1noc(-c2cnc(Nc3ccc(C(N)=O)c(Cl)c3)nc2N[C@H](CO)c2ccccc2)n1] | 2-chloro-4-[[5-(3-ethyl-1,2,4- oxadiazol-5-yl)-4-[[(1S)-2-hydroxy-1- phenyl-ethyl]amino]pyrimidin-2- yl]amino]benzamide | 480.2 | 480 |
| 242 | 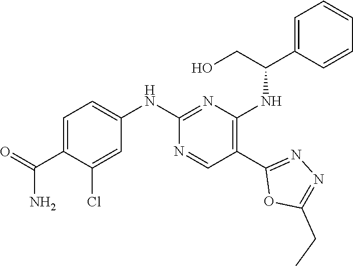 [SMILES: CCc1nnc(-c2cnc(Nc3ccc(C(N)=O)c(Cl)c3)nc2N[C@H](CO)c2ccccc2)o1] | 2-chloro-4-[[5-(5-ethyl-1,3,4- oxadiazol-2-yl)-4-[[(1S)-2-hydroxy-1- phenyl-ethyl]amino]pyrimidin-2- yl]amino]benzamide | 480.2 | 480 |
| 243 | 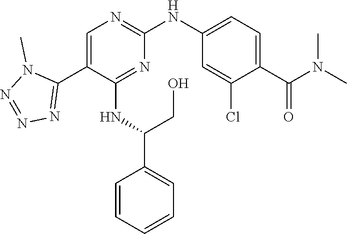 [SMILES: CN(C)C(=O)c1ccc(Nc2ncc(-c3nnnn3C)c(N[C@H](CO)c3ccccc3)n2)cc1Cl] | 2-chloro-4-[[4-[[(1S)-2-hydroxy-1- phenyl-ethyl]amino]-5-(1- methyltetrazol-5-yl)pyrimidin-2- yl]amino]-N-methyl-benzamide | 480.2 | 480 |
| 244 | 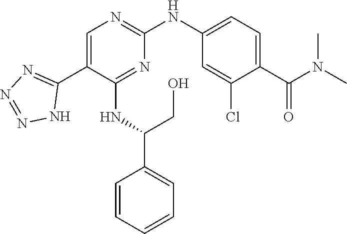 [SMILES: CN(C)C(=O)c1ccc(Nc2ncc(-c3nnn[nH]3)c(N[C@H](CO)c3ccccc3)n2)cc1Cl] | 2-chloro-4-[[4-[[(1S)-2-hydroxy-1- phenyl-ethyl]amino]-5-(1H-tetrazol-5- yl)pyrimidin-2-yl]amino]-N,N- dimethyl-benzamide | 480.2 | 480 |
| 245 | 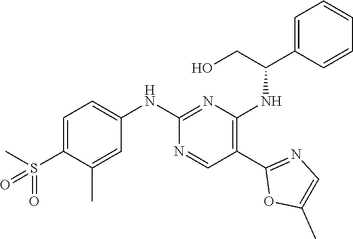 [SMILES: Cc1cnc(-c2cnc(Nc3ccc(S(C)(=O)=O)c(C)c3)nc2N[C@H](CO)c2ccccc2)o1] | (2S)-2-[[2-(3-methyl-4- methylsulfonyl-anilino)-5-(5- methyloxazol-2-yl)pyrimidin-4- yl]amino]-2-phenyl-ethanol | 480.2 | 480 |
| 246 | 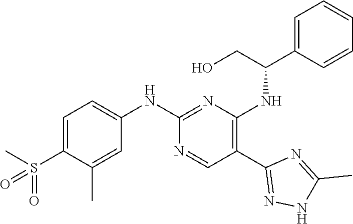 [SMILES: Cc1nc(-c2cnc(Nc3ccc(S(C)(=O)=O)c(C)c3)nc2N[C@H](CO)c2ccccc2)n[nH]1] | (2S)-2-[[2-(3-methyl-4- methylsulfonyl-anilino)-5-(5-methyl- 1H-1,2,4-triazol-3-yl)pyrimidin-4- yl]amino]-2-phenyl-ethanol | 480.2 | 480 |
| 247 | 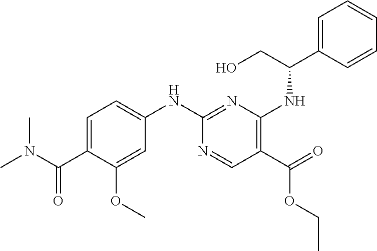 [SMILES: CCOC(=O)c1cnc(Nc2ccc(C(=O)N(C)C)c(OC)c2)nc1N[C@H](CO)c1ccccc1] | ethyl 2-[4-(dimethylcarbamoyl)-3- methoxy-anilino]-4-[[(1S)-2-hydroxy- 1-phenyl-ethyl]amino]pyrimidine-5- carboxylate | 480.2 | 480 |
| 248 | 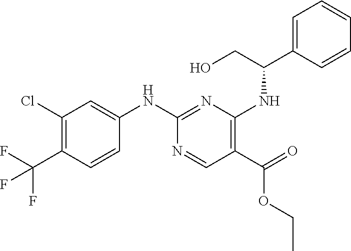 [SMILES: CCOC(=O)c1cnc(Nc2ccc(C(F)(F)F)c(Cl)c2)nc1N[C@H](CO)c1ccccc1] | ethyl 2-[3-chloro-4- (trifluoromethyl)anilino]-4-[[(1S)-2- hydroxy-1-phenyl- ethyl]amino]pyrimidine-5-carboxylate | 481.1 | 481 |
| 249 | 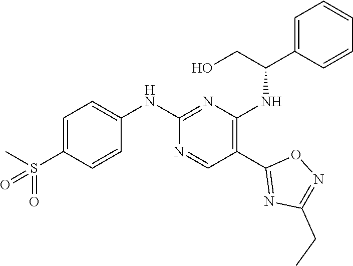 [SMILES: CCc1noc(-c2cnc(Nc3ccc(S(C)(=O)=O)cc3)nc2N[C@H](CO)c2ccccc2)n1] | (2S)-2-[[5-(3-ethyl-1,2,4-oxadiazol-5- yl)-2-(4-methylsulfonylanilino)- pyrimidin-4-yl]amino]-2-phenyl- ethanol | 481.2 | 481 |
| 250 | 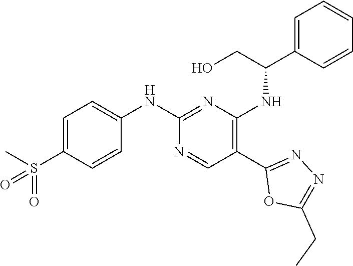 [SMILES: CCc1nnc(-c2cnc(Nc3ccc(S(C)(=O)=O)cc3)nc2N[C@H](CO)c2ccccc2)o1] | (2S)-2-[[5-(5-ethyl-1,3,4-oxadiazol-2- yl)-2-(4-methylsulfonyl- anilino)pyrimidin-4-yl]amino]-2- phenyl-ethanol | 481.2 | 481 |
| 251 | 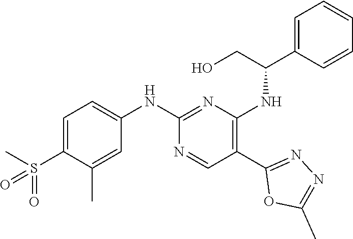 [SMILES: Cc1nnc(-c2cnc(Nc3ccc(S(C)(=O)=O)c(C)c3)nc2N[C@H](CO)c2ccccc2)o1] | (2S)-2-[[2-(3-methyl-4- methylsulfonyl-anilino)-5-(5-methyl- 1,3,4-oxadiazol-2-yl)pyrimidin-4- yl]amino]-2-phenyl-ethanol | 481.2 | 481 |
| 252 | 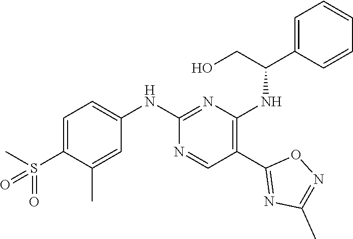 [SMILES: Cc1noc(-c2cnc(Nc3ccc(S(C)(=O)=O)c(C)c3)nc2N[C@H](CO)c2ccccc2)n1] | (2S)-2-[[2-(3-methyl-4- methyslulfonyl-anilino)-5-(3-methyl- 1,2,4-oxadiazol-5-yl)pyrimidin-4- yl]amino]-2-phenyl-ethanol | 481.2 | 481 |
| 253 | 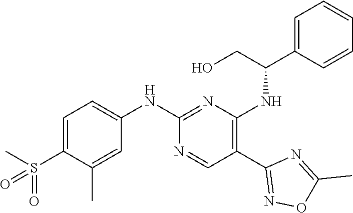 [SMILES: Cc1nc(-c2cnc(Nc3ccc(S(C)(=O)=O)c(C)c3)nc2N[C@H](CO)c2ccccc2)no1] | (2S)-2-[[2-(3-methyl-4- methylsulfonyl-anilino)-5-(5-methyl- 1,2,4-oxadiazol-3-yl)pyrimidin-4- yl]amino]-2-phenyl-ethanol | 481.2 | 481 |
| 254 | 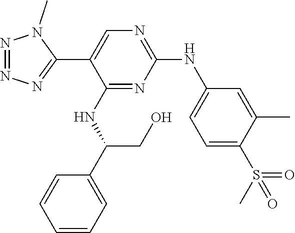 [SMILES: Cc1cc(Nc2ncc(-c3nnnn3C)c(N[C@H](CO)c3ccccc3)n2)ccc1S(C)(=O)=O] | (2S)-2-[[2-(3-methyl-4- methylsulfonyl-anilino)-5-(1- methyltetrazol-5-yl)pyrimidin-4- yl]amino]-2-phenyl-ethanol | 481.2 | 481 |
| 255 | 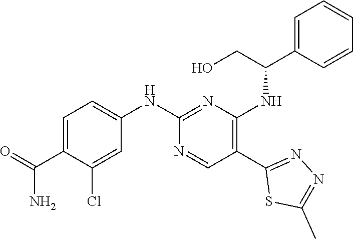 [SMILES: Cc1nnc(-c2cnc(Nc3ccc(C(N)=O)c(Cl)c3)nc2N[C@H](CO)c2ccccc2)s1] | 2-chloro-4-[[4-[[(1S)-2-hydroxy-1- phenyl-ethyl]amino]-5-(5-methyl- 1,3,4-thiadiazol-2-yl)pyrimidin-2- yl]amino]benzamide | 482.1 | 482 |
| 256 | 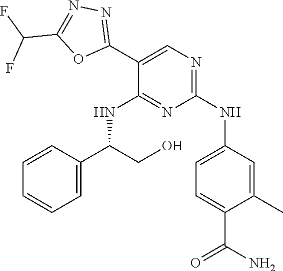 [SMILES: Cc1cc(Nc2ncc(-c3nnc(C(F)F)o3)c(N[C@H](CO)c3ccccc3)n2)ccc1C(N)=O] | 4-[[5-[5-(difluoromethyl)-1,3,4- oxadiazol-2-yl]-4-[[(1S)-2-hydroxy-1- phenyl-ethyl]amino]pyrimidin-2- yl]amino]-2-methyl-benzamide | 482.2 | 482 |
| 257 | 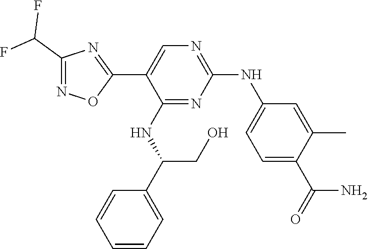 [SMILES: Cc1cc(Nc2ncc(-c3nc(C(F)F)no3)c(N[C@H](CO)c3ccccc3)n2)ccc1C(N)=O] | 4-[[5-[3-(difluoromethyl)-1,2,4- oxadiazol-5-yl]-4-[[(1S)-2-hydroxy-1- phenyl-ethyl]amino]pyrimidin-2- yl]amino]-2-methyl-benzamide | 482.2 | 482 |
| 258 | 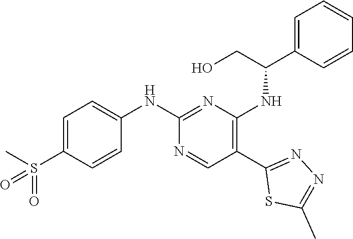 [SMILES: Cc1nnc(-c2cnc(Nc3ccc(S(C)(=O)=O)cc3)nc2N[C@H](CO)c2ccccc2)s1] | (2S)-2-[[2-(4-methylsulfonylanilino)- 5-(5-methyl-1,3,4-thiadiazol-2- yl)pyrimidin-4-yl]amino]-2-phenyl- ethanol | 483.1 | 483 |
| 259 | 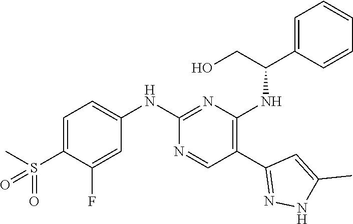 [SMILES: Cc1cc(-c2cnc(Nc3ccc(S(C)(=O)=O)c(F)c3)nc2N[C@H](CO)c2ccccc2)n[nH]1] | (2S)-2-[[2-(3-fluoro-4- methylsulfonyl-anilino)-5-(5-methyl- 1H-pyrazol-3-yl)pyrimidin-4- yl]amino]-2-phenyl-ethanol | 483.2 | 483 |
| 260 | 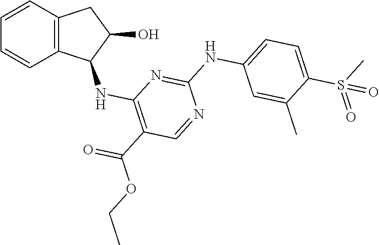 [SMILES: CCOC(=O)c1cnc(Nc2ccc(S(C)(=O)=O)c(C)c2)nc1N[C@H]1c2ccccc2C[C@H]1O] | ethyl 4-[[(1S,2R)-2-hydroxyindan-1- yl]amino]-2-(3-methyl-4- methylsulfonyl-anilino)pyrimidine-5- carboxylate | 483.2 | 483 |
| 261 | 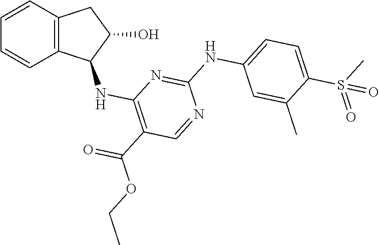 [SMILES: CCOC(=O)c1cnc(Nc2ccc(S(C)(=O)=O)c(C)c2)nc1N[C@H]1c2ccccc2C[C@@H]1O] | ethyl 4-[[(1S,2S)-2-hydroxyindan-1- yl]amino]-2-(3-methyl-4- methylsulfonyl-anilino)pyrimidine-5- carboxylate | 483.2 | 483 |
| 262 | 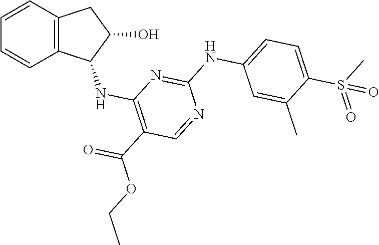 [SMILES: CCOC(=O)c1cnc(Nc2ccc(S(C)(=O)=O)c(C)c2)nc1N[C@@H]1c2ccccc2C[C@@H]1O] | ethyl 4-[[(1R,2S)-2-hydroxyindan-1- yl]amino]-2-(3-methyl-4- methylsulfonyl-anilino)pyrimidine-5- carboxylate | 483.2 | 483 |
| 263 | 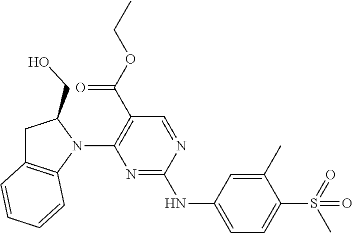 [SMILES: CCOC(=O)c1cnc(Nc2ccc(S(C)(=O)=O)c(C)c2)nc1N1c2ccccc2C[C@H]1CO] | ethyl 4-[(2S)-2-(hydroxymethyl)- indolin-1-yl]-2-(3-methyl-4- methylsulfonyl-anilino)pyrimidine-5- carboxylate | 483.2 | 483 |
| 264 | 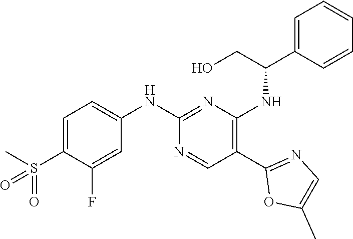 [SMILES: Cc1cnc(-c2cnc(Nc3ccc(S(C)(=O)=O)c(F)c3)nc2N[C@H](CO)c2ccccc2)o1] | (2S)-2-[[2-(3-fluoro-4- methylsulfonyl-anilino)-5-(5- methyloxazol-2-yl)pyrimidin-4- yl]amino]-2-phenyl-ethanol | 484.1 | 484 |
| 265 | 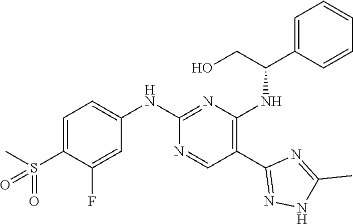 [SMILES: Cc1nc(-c2cnc(Nc3ccc(S(C)(=O)=O)c(F)c3)nc2N[C@H](CO)c2ccccc2)n[nH]1] | (2S)-2-[[2-(3-fluoro-4- methylsulfonyl-anilino)-5-(5-methyl- 1H-1,2,4-triazol-3-yl)pyrimidin-4- yl]amino]-2-phenyl-ethanol | 484.2 | 484 |
| 266 | 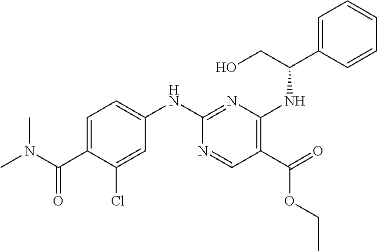 [SMILES: CCOC(=O)c1cnc(Nc2ccc(C(=O)N(C)C)c(Cl)c2)nc1N[C@H](CO)c1ccccc1] | ethyl 2-[3-chloro-4- (dimethylcarbamoyl)anilino]-4-[[(1S)- 2-hydroxy-1-phenyl- ethyl]amino]pyrimidine-5-carboxylate | 484.2 | 484 |
| 267 | 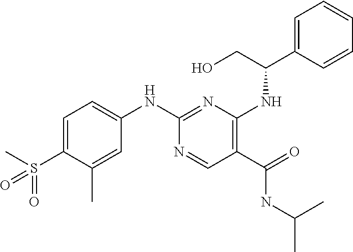 | 4-[[(1S)-2-hydroxy-1-phenyl- ethyl]amino]-N-isopropyl-2-(3- methyl-4-methylsulfonyl- anilino)pyrimidine-5-carboxamide | 484.2 | 484 |
| 268 | 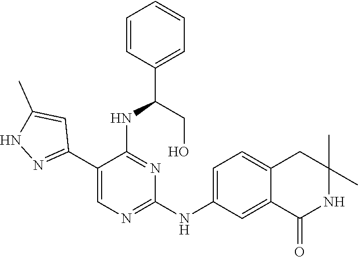 [SMILES: Cc1cc(-c2cnc(Nc3ccc4c(c3)C(=O)NC(C)(C)C4)nc2N[C@H](CO)c2ccccc2)n[nH]1] | 7-[[4-[[(1S)-2-hydroxy-1-phenyl- ethyl]amino]-5-(5-methyl-1H- pyrazol-3-yl)pyrimidin-2-yl]amino]- 3,3-dimethyl-2,4-dihydroisoquinolin- 1-one | 484.2 | 484 |
| 269 | 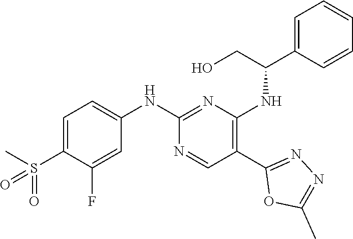 [SMILES: Cc1nnc(-c2cnc(Nc3ccc(S(C)(=O)=O)c(F)c3)nc2N[C@H](CO)c2ccccc2)o1] | (2S)-2-[[2-(3-fluoro-4- methylsulfonyl-anilino)-5-(5-methyl- 1,3,4-oxadiazol-2-yl)pyrimidin-4- yl]amino]-2-phenyl-ethanol | 485.1 | 485 |
| 270 | 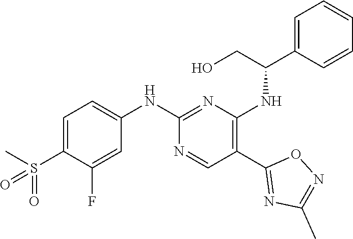 [SMILES: Cc1noc(-c2cnc(Nc3ccc(S(C)(=O)=O)c(F)c3)nc2N[C@H](CO)c2ccccc2)n1] | (2S)-2-[[2-(3-fluoro-4- methylsulfonyl-anilino)-5-(3-methyl- 1,2,4-oxadiazol-5-yl)pyrimidin-4- yl]amino]-2-phenyl-ethanol | 485.1 | 485 |
| 271 | 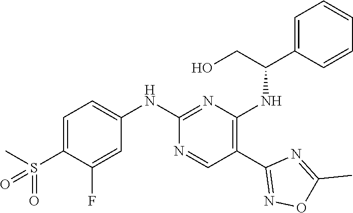 [SMILES: Cc1nc(-c2cnc(Nc3ccc(S(C)(=O)=O)c(F)c3)nc2N[C@H](CO)c2ccccc2)no1] | (2S)-2-[[2-(3-fluoro-4- methylsulfonyl-anilino)-5-(5-methyl- 1,2,4-oxadiazol-3-yl)pyrimidin-4- yl]amino]-2-phenyl-ethanol | 485.1 | 485 |
| 272 | 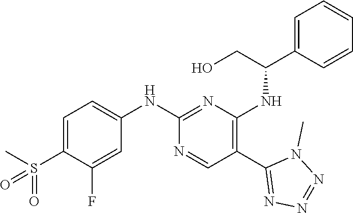 [SMILES: Cn1nnnc1-c1cnc(Nc2ccc(S(C)(=O)=O)c(F)c2)nc1N[C@H](CO)c1ccccc1] | (2S)-2-[[2-(3-fluoro-4- methylsulfonyl-anilino)-5-(1- methyltetrazol-5-yl)pyrimidin-4- yl]amino]-2-phenyl-ethanol | 485.2 | 485 |
| 273 | 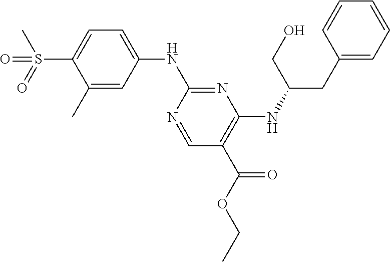 [SMILES: CCOC(=O)c1cnc(Nc2ccc(S(C)(=O)=O)c(C)c2)nc1N[C@H](CO)Cc1ccccc1] | ethyl 4-[[(1S)-1-benzyl-2-hydroxy- ethyl]amino]-2-(3-methyl-4- methylsulfonyl-anilino)pyrimidine-5- carboxylate | 485.2 | 485 |
| 274 | 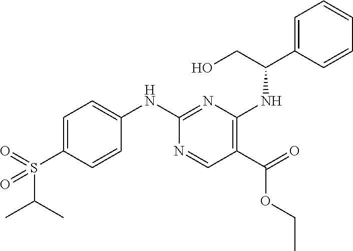 [SMILES: CCOC(=O)c1cnc(Nc2ccc(S(=O)(=O)C(C)C)cc2)nc1N[C@H](CO)c1ccccc1] | ethyl 4-[[(1S)-2-hydroxy-1-phenyl- ethyl]amino]-2-(4- isopropylsulfonylanilino)pyrimidine- 5-carboxylate | 485.2 | 485 |
| 275 | 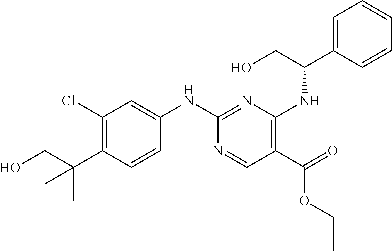 [SMILES: CCOC(=O)c1cnc(Nc2ccc(C(C)(C)CO)c(Cl)c2)nc1N[C@H](CO)c1ccccc1] | ethyl 2-[3-chloro-4-(2-hydroxy-1,1- dimethyl-ethyl)anilino]-4-[[(1S)-2- hydroxy-1-phenyl- ethyl]amino]pyrimidine-5-carboxylate | 485.2 | 485 |
| 276 | 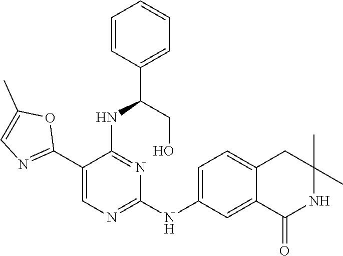 [SMILES: Cc1cnc(-c2cnc(Nc3ccc4c(c3)C(=O)NC(C)(C)C4)nc2N[C@H](CO)c2ccccc2)o1] | 7-[[4-[[(1S)-2-hydroxy-1-phenyl- ethyl]amino]-5-(5-methyloxazol-2- yl)pyrimidin-2-yl]amino]-3,3- dimethyl-2,4-dihydroisoquinolin-1- one | 485.2 | 485 |
| 277 | 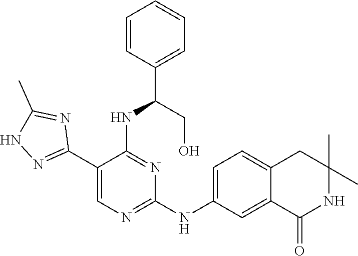 [SMILES: Cc1nc(-c2cnc(Nc3ccc4c(c3)C(=O)NC(C)(C)C4)nc2N[C@H](CO)c2ccccc2)n[nH]1] | 7-[[4-[[(1S)-2-hydroxy-1-phenyl- ethyl]amino]-5-(5-methyl-1H-1,2,4- triazol-3-yl)pyrimidin-2-yl]amino]- 3,3-dimethyl-2,4-dihydroisoquinolin- 1-one | 485.2 | 485 |
| 278 | 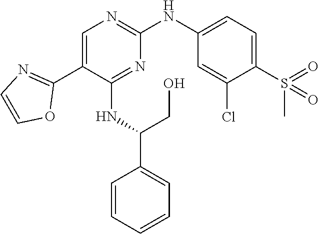 [SMILES: CS(=O)(=O)c1ccc(Nc2ncc(-c3ncco3)c(N[C@H](CO)c3ccccc3)n2)cc1Cl] | (2S)-2-[[2-(3-chloro-4- methylsulfonyl-anilino)-5-oxazol-2- yl-pyrimidin-4-yl]amino]-2-phenyl- ethanol | 486.1 | 486 |
| 279 | 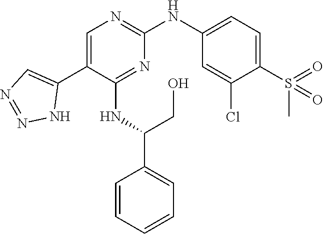 [SMILES: CS(=O)(=O)c1ccc(Nc2ncc(-c3cnn[nH]3)c(N[C@H](CO)c3ccccc3)n2)cc1Cl] | (2S)-2-[[2-(3-chloro-4- methylsulfonyl-anilino)-5-(1H-triazol- 5-yl)pyrimidin-4-yl]amino]-2-phenyl- ethanol | 486.1 | 486 |
| 280 | 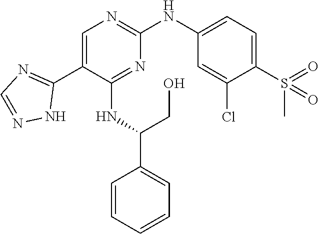 [SMILES: CS(=O)(=O)c1ccc(Nc2ncc(-c3ncn[nH]3)c(N[C@H](CO)c3ccccc3)n2)cc1Cl] | (2S)-2-[[2-(3-chloro-4- methylsulfonyl-anilino)-5-(1H-1,2,4- triazol-5-yl)pyrimidin-4-yl]amino]-2- phenyl-ethanol | 486.1 | 486 |
| 281 | 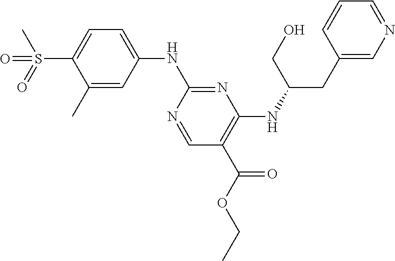 [SMILES: CCOC(=O)c1cnc(Nc2ccc(S(C)(=O)=O)c(C)c2)nc1N[C@H](CO)Cc1cccnc1] | ethyl 4-[[(1S)-1-(hydroxymethyl)-2- (3-pyridyl)ethyl]amino]-2-(3-methyl- 4-methylsulfonyl-anilino)pyrimidine- 5-carboxylate | 486.2 | 486 |
| 282 | 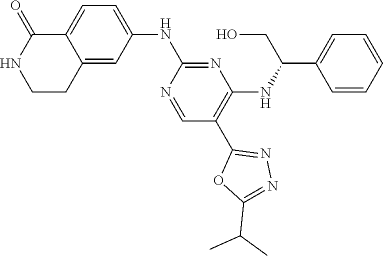 [SMILES: CC(C)c1nnc(-c2cnc(Nc3ccc4c(c3)CCNC4=O)nc2N[C@H](CO)c2ccccc2)o1] | 6-[[4-[[(1S)-2-hydroxy-1-phenyl- ethyl]amino]-5-(5-isopropyl-1,3,4- oxadiazol-2-yl)pyrimidin-2- yl]amino]-3,4-dihydro-2H- isoquinolin-1-one | 486.2 | 486 |
| 283 | 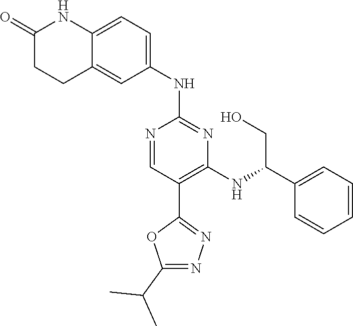 [SMILES: CC(C)c1nnc(-c2cnc(Nc3ccc4c(c3)CCC(=O)N4)nc2N[C@H](CO)c2ccccc2)o1] | 6-[[4-[[(1S)-2-hydroxy-1-phenyl- ethyl]amino]-5-(5-isopropyl-1,3,4- oxadiazol-2-yl)pyrimidin-2- yl]amino]-3,4-dihydro-1H-quinolin-2- one | 486.2 | 486 |
| 284 | 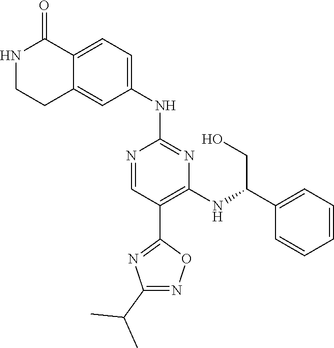 [SMILES: CC(C)c1noc(-c2cnc(Nc3ccc4c(c3)CCNC4=O)nc2N[C@H](CO)c2ccccc2)n1] | 6-[[4-[[(1S)-2-hydroxy-1-phenyl- ethyl]amino]-5-(3-isopropyl-1,2,4- oxadiazol-5-yl)pyrimidin-2- yl]amino]-3,4-dihydro-2H- isoquinolin-1-one | 486.2 | 486 |
| 285 | 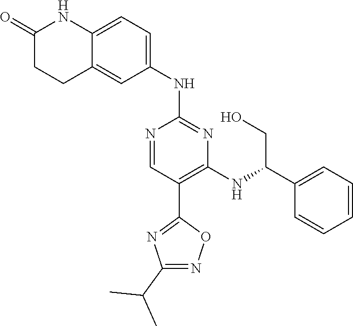 [SMILES: CC(C)c1noc(-c2cnc(Nc3ccc4c(c3)CCC(=O)N4)nc2N[C@H](CO)c2ccccc2)n1] | 6-[[4-[[(1S)-2-hydroxy-1-phenyl- ethyl]amino]-5-(3-isopropyl-1,2,4- oxadiazol-5-yl)pyrimidin-2- yl]amino]-3,4-dihydro-1H-quinolin-2- one | 486.2 | 486 |
| 286 | 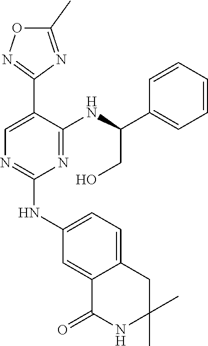 [SMILES: Cc1nc(-c2cnc(Nc3ccc4c(c3)C(=O)NC(C)(C)C4)nc2N[C@H](CO)c2ccccc2)no1] | 7-[[4-[[(1S)-2-hydroxy-1-phenyl- ethyl]amino]-5-(5-methyl-1,2,4- oxadiazol-3-yl)pyrimidin-2- yl]amino]-3,3-dimethyl-2,4- dihydroisoquinolin-1-one | 486.2 | 486 |
| 287 | 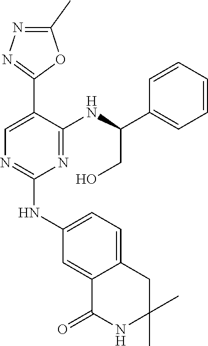 [SMILES: Cc1nnc(-c2cnc(Nc3ccc4c(c3)C(=O)NC(C)(C)C4)nc2N[C@H](CO)c2ccccc2)o1] | 7-[[4-[[(1S)-2-hydroxy-1-phenyl- ethyl]amino]-5-(5-methyl-1,3,4- oxadiazol-2-yl)pyrimidin-2- yl]amino]-3,3-dimethyl-2,4- dihydroisoquinolin-1-one | 486.2 | 486 |
| 288 | 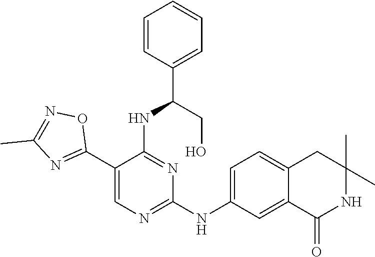 | 7-[[4-[[(1S)-2-hydroxy-1-phenyl- ethyl]amino]-5-(3-methyl-1,2,4- oxadiazol-5-yl)pyrimidin-2- yl]amino]-3,3-dimethyl-2,4- dihydroisoquinolin-1-one | 486.2 | 486 |
| 289 | 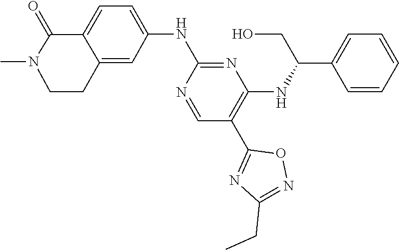 [SMILES: CCc1noc(-c2cnc(Nc3ccc4c(c3)CCN(C)C4=O)nc2N[C@H](CO)c2ccccc2)n1] | 6-[[5-(3-ethyl-1,2,4-oxadiazol-5-yl)- 4-[[(1S)-2-hydroxy-1-phenyl- ethyl]amino]pyrimidin-2-yl]amino]-2- methyl-3,4-dihydroisoquinolin-1-one | 486.2 | 486 |
| 290 | 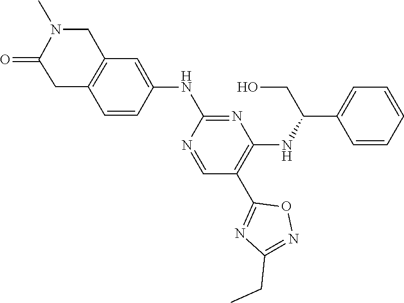 [SMILES: CCc1noc(-c2cnc(Nc3ccc4c(c3)CN(C)C(=O)C4)nc2N[C@H](CO)c2ccccc2)n1] | 7-[[5-(3-ethyl-1,2,4-oxadiazol-5-yl)- 4-[[(1S)-2-hydroxy-1-phenyl- ethyl]amino]pyrimidin-2-yl]amino]-2- methyl-1,4-dihydroisoquinolin-3-one | 486.2 | 486 |
| 291 | 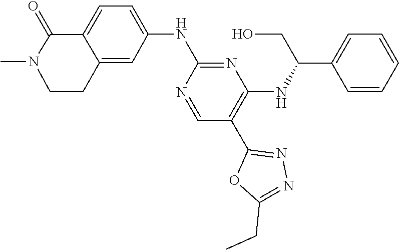 [SMILES: CCc1nnc(-c2cnc(Nc3ccc4c(c3)CCN(C)C4=O)nc2N[C@H](CO)c2ccccc2)o1] | 6-[[5-(5-ethyl-1,3,4-oxadiazol-2-yl)- 4-[[(1S)-2-hydroxy-1-phenyl- ethyl]amino]pyrimidin-2-yl]amino]-2- methyl-3,4-dihydroisoquinolin-1-one | 486.2 | 486 |
| 292 | 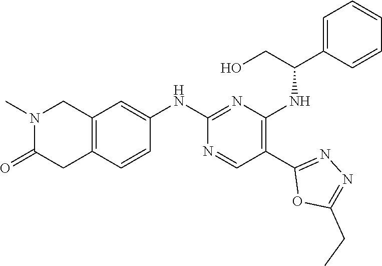 [SMILES: CCc1nnc(-c2cnc(Nc3ccc4c(c3)CN(C)C(=O)C4)nc2N[C@H](CO)c2ccccc2)o1] | 7-[[5-(5-ethyl-1,3,4-oxadiazol-2-yl)- 4-[[(1S)-2-hydroxy-1-phenyl- ethyl]amino]pyrimidin-2-yl]amino]-2- methyl-1,4-dihydroisoquinolin-3-one | 486.2 | 486 |
| 293 | 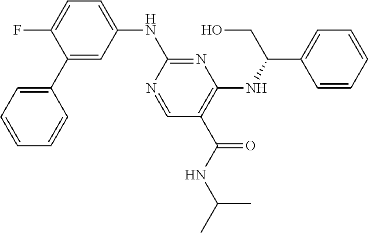 [SMILES: CC(C)NC(=O)c1cnc(Nc2ccc(F)c(-c3ccccc3)c2)nc1N[C@H](CO)c1ccccc1] | 2-(4-fluoro-3-phenyl-anilino)-4- [[(1S)-2-hydroxy-1-phenyl- ethyl]amino]-N-isopropyl-pyrimidine- 5-carboxamide | 486.2 | 486 |
| 294 | 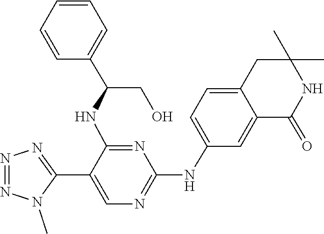 [SMILES: Cn1nnnc1-c1cnc(Nc2ccc3c(c2)C(=O)NC(C)(C)C3)nc1N[C@H](CO)c1ccccc1] | 7-[[4-[[(1S)-2-hydroxy-1-phenyl- ethyl]amino]-5-(1-methyltetrazol-5- yl)pyrimidin-2-yl]amino]-3,3- dimethyl-2,4-dihydroisoquinolin-1- one | 486.2 | 486 |
| 295 | 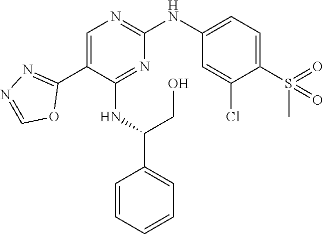 [SMILES: CS(=O)(=O)c1ccc(Nc2ncc(-c3nnco3)c(N[C@H](CO)c3ccccc3)n2)cc1Cl] | (2S)-2-[[2-(3-chloro-4- methylsulfonyl-anilino)-5-(1,3,4- oxadiazol-2-yl)pyrimidin-4- yl]amino]-2-phenyl-ethanol | 487.1 | 487 |
| 296 | 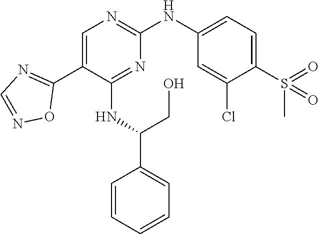 [SMILES: CS(=O)(=O)c1ccc(Nc2ncc(-c3ncno3)c(N[C@H](CO)c3ccccc3)n2)cc1Cl] | (2S)-2-[[2-(3-chloro-4- methylsulfonyl-anilino)-5-(1,2,4- oxadiazol-5-yl)pyrimidin-4- yl]amino]-2-phenyl-ethanol | 487.1 | 487 |
| 297 | 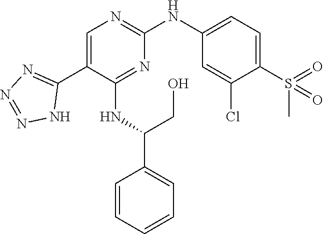 [SMILES: CS(=O)(=O)c1ccc(Nc2ncc(-c3nnn[nH]3)c(N[C@H](CO)c3ccccc3)n2)cc1Cl] | (2S)-2-[[2-(3-chloro-4- methylsulfonyl-anilino)-5-(1H- tetrazol-5-yl)pyrimidin-4-yl]amino]- 2-phenyl-ethanol | 487.1 | 487 |
| 298 | 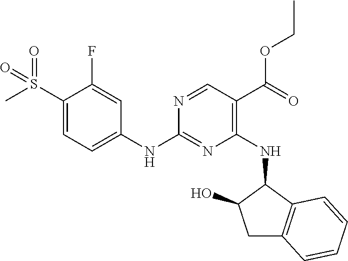 [SMILES: CCOC(=O)c1cnc(Nc2ccc(S(C)(=O)=O)c(F)c2)nc1N[C@H]1c2ccccc2C[C@H]1O] | ethyl 2-(3-fluoro-4-methylsulfonyl- anilino)-4-[[(1S,2R)-2-hydroxyindan- 1-yl]amino]pyrimidine-5-carboxylate | 487.1 | 487 |
| 299 | 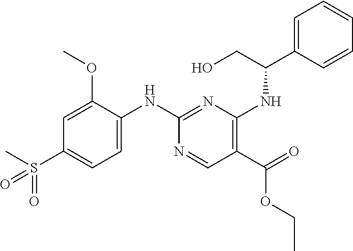 [SMILES: CCOC(=O)c1cnc(Nc2ccc(S(C)(=O)=O)cc2OC)nc1N[C@H](CO)c1ccccc1] | ethyl 4-[[(1S)-2-hydroxy-1-phenyl- ethyl]amino]-2-(2-methoxy-4- methylsulfonyl-anilino)pyrimidine-5- carboxylate | 487.2 | 487 |
| 300 | 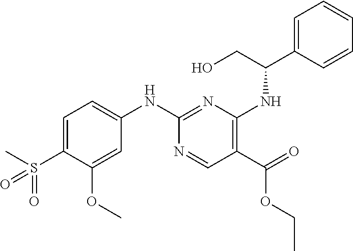 [SMILES: CCOC(=O)c1cnc(Nc2ccc(S(C)(=O)=O)c(OC)c2)nc1N[C@H](CO)c1ccccc1] | ethyl 4-[[(1S)-2-hydroxy-1-phenyl- ethyl]amino]-2-(3-methoxy-4- methylsulfonyl-anilino)pyrimidine-5- carboxylate | 487.2 | 487 |
| 301 | 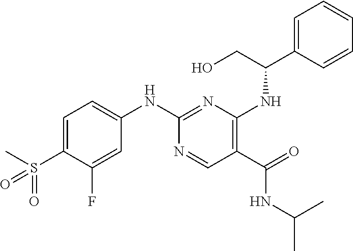 [SMILES: CC(C)NC(=O)c1cnc(Nc2ccc(S(C)(=O)=O)c(F)c2)nc1N[C@H](CO)c1ccccc1] | 2-(3-fluoro-4-methylsulfonyl- anilino)-4-[[(1S)-2-hydroxy-1-phenyl- ethyl]amino]-N-isopropyl-pyrimidine- 5-carboxamide | 488.2 | 488 |
| 302 | 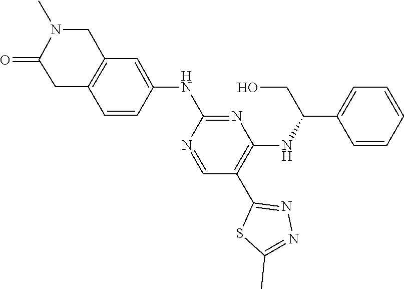 [SMILES: Cc1nnc(-c2cnc(Nc3ccc4c(c3)CN(C)C(=O)C4)nc2N[C@H](CO)c2ccccc2)s1] | 7-[[4-[[(1S)-2-hydroxy-1-phenyl- ethyl]amino]-5-(5-methyl-1,3,4- thiadiazol-2-yl)pyrimidin-2- yl]amino]-2-methyl-1,4- dihydroisoquinolin-3-one | 488.2 | 488 |
| 303 | 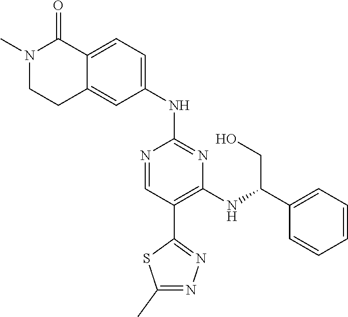 [SMILES: Cc1nnc(-c2cnc(Nc3ccc4c(c3)CCN(C)C4=O)nc2N[C@H](CO)c2ccccc2)s1] | 6-[[4-[[(1S)-1-hydroxy-1-phenyl- ethyl]amino]-5-(5-methyl-1,3,4- thiadiazol-2-yl)pyrimidin-2- yl]amino]-2-methyl-3,4- dihydroisoquinolin-1-one | 488.2 | 488 |
| 304 | 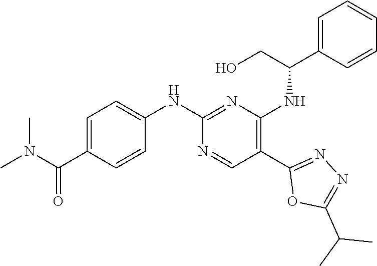 [SMILES: CC(C)c1nnc(-c2cnc(Nc3ccc(C(=O)N(C)C)cc3)nc2N[C@H](CO)c2ccccc2)o1] | 4-[[4-[[(1S)-2-hydroxy-1-phenyl- ethyl]amino]-5-(5-isopropyl-1,3,4- oxadiazol-2-yl)pyrimidin-2- yl]amino]-N,N-dimethyl-benzamide | 488.2 | 488 |
| 305 | 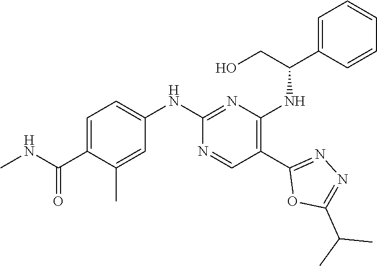 [SMILES: CNC(=O)c1ccc(Nc2ncc(-c3nnc(C(C)C)o3)c(N[C@H](CO)c3ccccc3)n2)cc1C] | 4-[[4-[[(1S)-2-hydroxy-1-phenyl- ethyl]amino]-5-(5-isopropyl-1,3,4- oxadiazol-2-yl)pyrimidin-2- yl]amino]-N,2-dimethyl-benzamide | 488.2 | 488 |
| 306 | 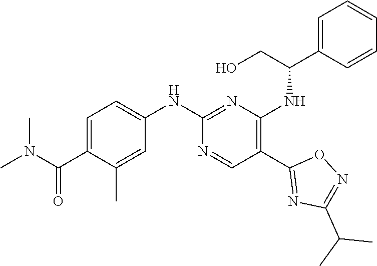 [SMILES: Cc1cc(Nc2ncc(-c3nc(C(C)C)no3)c(N[C@H](CO)c3ccccc3)n2)ccc1C(=O)N(C)C] | 4-[[4-[[(1S)-2-hydroxy-1-phenyl- ethyl]amino]-5-(3-isopropyl-1,2,4- oxadiazol-5-yl)pyrimidin-2- yl]amino]-N,N-dimethyl-benzamide | 488.2 | 488 |
| 307 | 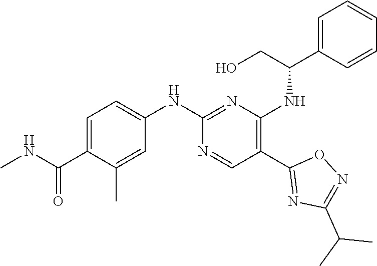 [SMILES: CNC(=O)c1ccc(Nc2ncc(-c3nc(C(C)C)no3)c(N[C@H](CO)c3ccccc3)n2)cc1C] | 4-[[4-[[(1S)-2-hydroxy-1phenyl- ethyl]amino]-5-(3-isopropyl-1,2,4- oxadiazol-5-yl)pyrimidin-2- yl]amino]-N,2-dimethyl-benzamide | 488.2 | 488 |
| 308 | 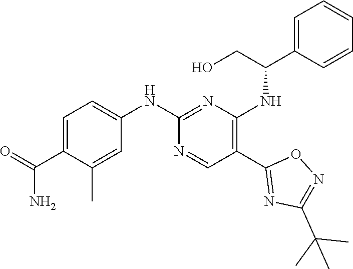 [SMILES: Cc1cc(Nc2ncc(-c3nc(C(C)(C)C)no3)c(N[C@H](CO)c3ccccc3)n2)ccc1C(N)=O] | 4-[[5-(3-tert-butyl-1,2,4-oxadiazol-5- yl)-4-[[(1S)-2-hydroxy-1-phenyl- ethyl]amino]pyrimidin-2-yl]amino]-2- methyl-benzamide | 488.2 | 488 |
| 309 | 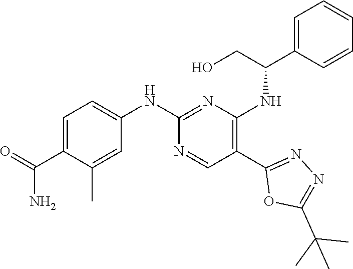 [SMILES: Cc1cc(Nc2ncc(-c3nnc(C(C)(C)C)o3)c(N[C@H](CO)c3ccccc3)n2)ccc1C(N)=O] | 4-[[5-(5-tert-butyl-1,3,4-oxadiazol-2- yl)-4-[[(1S)-2-hydroxy-1-phenyl- ethyl]amino]pyrimidin-2-yl]amino]-2- methyl-benzamide | 488.2 | 488 |
| 310 | 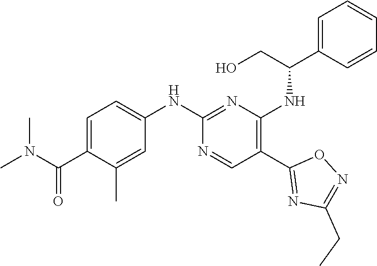 [SMILES: CCc1noc(-c2cnc(Nc3ccc(C(=O)N(C)C)c(C)c3)nc2N[C@H](CO)c2ccccc2)n1] | 4-[[5-(3-ethyl-1,2,4-oxadiazol-5-yl)- 4-[[(1S)-2-hydroxy-1-phenyl- ethyl]amino]pyrimidin-2-yl]amino]- N,N,2-trimethyl-benzamide | 488.2 | 488 |
| 311 | 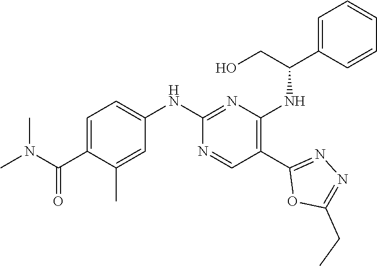 [SMILES: CCc1nnc(-c2cnc(Nc3ccc(C(=O)N(C)C)c(C)c3)nc2N[C@H](CO)c2ccccc2)o1] | 4-[[5-(5-ethyl-1,3,4-oxadiazol-2-yl)- 4-[[(1S)-2-hydroxy-1-phenyl- ethyl]amino]pyrimidin-2-yl]amino]- N,N,2-trimethyl-benzamide | 488.2 | 488 |
| 312 | 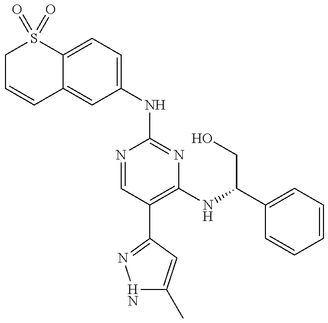 [SMILES: Cc1cc(-c2cnc(Nc3ccc4c(c3)C=CCS4(=O)=O)nc2N[C@H](CO)c2ccccc2)n[nH]1] | (2S)-2-[[2-[(1,1-dioxo-2H- thiochromen-6-yl)amino]-5-(5- methyl-1H-pyrazol-3-yl)pyrimidin-4- yl]amino]-2-phenyl-ethanol | 489.2 | 489 |
| 313 | 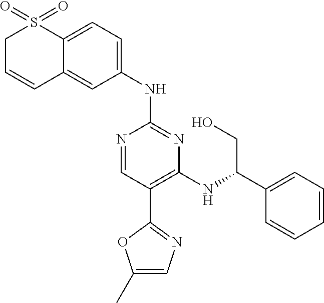 [SMILES: Cc1cnc(-c2cnc(Nc3ccc4c(c3)C=CCS4(=O)=O)nc2N[C@H](CO)c2ccccc2)o1] | (2S)-2-[[2-[(1,1-dioxo-2H- thiochromen-6-yl)amino]-5-(5- methyloxazol-2-yl)pyrimidin-4- yl]amino]-2-phenyl-ethanol | 490.2 | 490 |
| 314 | 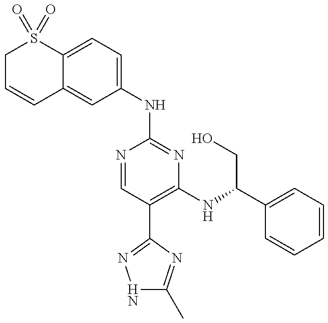 [SMILES: Cc1nc(-c2cnc(Nc3ccc4c(c3)C=CCS4(=O)=O)nc2N[C@H](CO)c2ccccc2)n[nH]1] | (2S)-2-[[2-[(1,1-dioxo-2H- thiochromen-6-yl)amino]-5-(5- methyl-1H-1,2,4-triazol-3- yl)pyrimidin-4-yl]amino]-2-phenyl- ethanol | 490.2 | 490 |
| 315 | 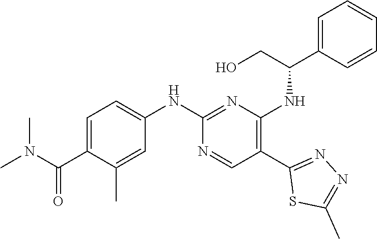 [SMILES: Cc1nnc(-c2cnc(Nc3ccc(C(=O)N(C)C)c(C)c3)nc2N[C@H](CO)c2ccccc2)s1] | 4-[[4-[[(1S)-2-hydroxy-1-phenyl- ethyl]amino]-5-(5-methyl-1,3,4- thiadiazol-2-yl)pyrimidin-2- yl]amino]-N,N,2-trimethyl-benzamide | 490.2 | 490 |
| 316 | 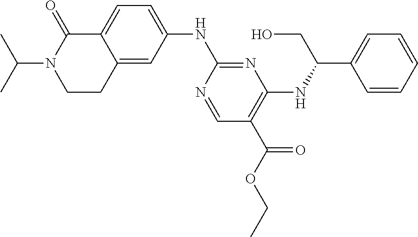 [SMILES: CCOC(=O)c1cnc(Nc2ccc3c(c2)CCN(C(C)C)C3=O)nc1N[C@H](CO)c1ccccc1] | ethyl 4-[[(1S)-2-hydroxy-1-phenyl- ethyl]amino]-2-[(2-isopropyl-1-oxo- 3,4-dihydroisoquinolin-6- yl)amino]pyrimidin-5-carboxylate | 490.2 | 490 |
| 317 | 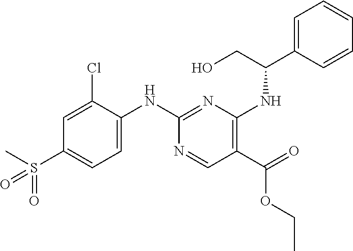 [SMILES: CCOC(=O)c1cnc(Nc2ccc(S(C)(=O)=O)cc2Cl)nc1N[C@H](CO)c1ccccc1] | ethyl 2-(2-chloro-4-methylsulfonyl- anilino)-4-[[(1S)-2-hydroxy-1-phenyl- ethyl]amino]pyrimidine-5-carboxylate | 491.1 | 491 |
| 318 | 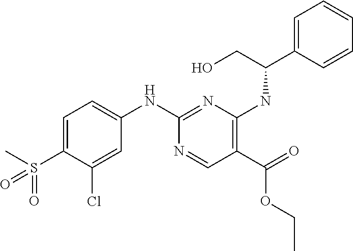 | ethyl 2-(3-chloro-4-methylsulfonyl- anilino)-4-[[(1S)-2-hydroxy-1-phenyl- ethyl]amino]-pyrimidine-5- carboxylate | 491.1 | 491 |
| 319 | 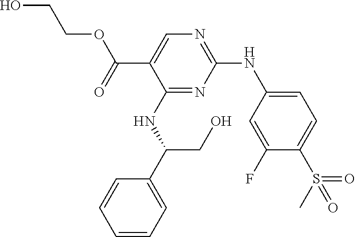 [SMILES: CS(=O)(=O)c1ccc(Nc2ncc(C(=O)OCCO)c(N[C@H](CO)c3ccccc3)n2)cc1F] | 2-hydroxyethyl 2-(3-fluoro-4- methylsulfonyl-anilino)-4-[[(1S)-2- hydroxy-1-phenyl- ethyl]amino]pyrimidine-5-carboxylate | 491.1 | 491 |
| 320 | 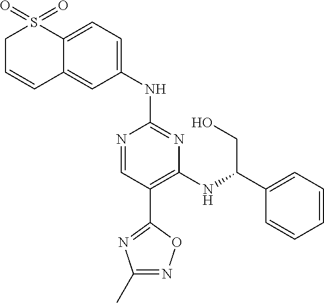 [SMILES: Cc1noc(-c2cnc(Nc3ccc4c(c3)C=CCS4(=O)=O)nc2N[C@H](CO)c2ccccc2)n1] | (2S)-2-[[2-[(1,1-dioxo-2H- thiochromen-6-yl)amino]-5-(3- methyl-1,2,4-oxadiazol-5- yl)pyrimidin-4-yl]amino]-2-phenyl- ethanol | 491.2 | 491 |
| 321 | 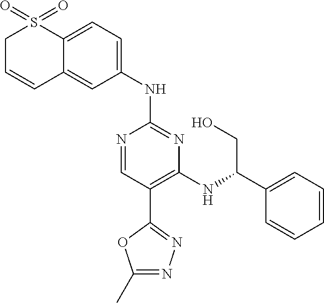 [SMILES: Cc1nnc(-c2cnc(Nc3ccc4c(c3)C=CCS4(=O)=O)nc2N[C@H](CO)c2ccccc2)o1] | (2S)-2-[[2-[(1,1-dioxo-2H- thiochromen-6-yl)amino]-5-(5- methyl-1,3,4-oxadiazol-2- yl)pyrimidin-4-yl]amino]-2-phenyl- ethanol | 491.2 | 491 |
| 322 | 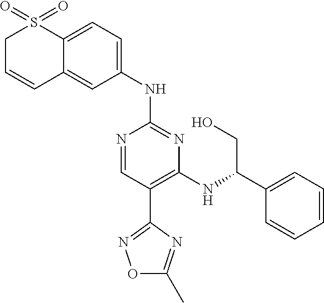 [SMILES: Cc1nc(-c2cnc(Nc3ccc4c(c3)C=CCS4(=O)=O)nc2N[C@H](CO)c2ccccc2)no1] | (2S)-2-[[2-[(1,1-dioxo-2H- thiochromen-6-yl)amino]-5-(5- methyl-1,2,4-oxadiazol-3- yl)pyrimidin-4-yl]amino]-2-phenyl- ethanol | 491.2 | 491 |
| 323 | 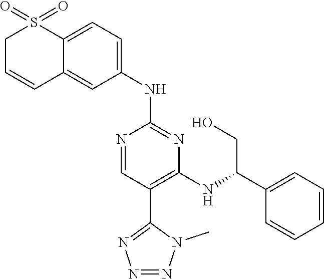 [SMILES: Cn1nnnc1-c1cnc(Nc2ccc3c(c2)C=CCS3(=O)=O)nc1N[C@H](CO)c1ccccc1] | (2S)-2-[[2-[(1,1-dioxo-2H- thiochromen-6-yl)amino]-5-(1- methyltetrazol-5-yl)pyrimidin-4- yl]amino]-2-phenyl-ethanol | 491.2 | 491 |
| 324 | 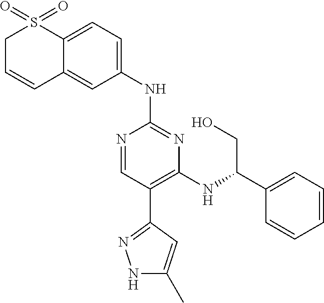 [SMILES: Cc1cc(-c2cnc(Nc3ccc4c(c3)C=CCS4(=O)=O)nc2N[C@H](CO)c2ccccc2)n[nH]1] | (2S)-2-[[2-[(1,1-dioxo-3,4-dihydro- 2H-thiochromen-6-yl)amino]-5-(5- methyl-1H-pyrazol-3-yl)pyrimidin-4- yl]amino]-2-phenyl-ethanol | 491.2 | 491 |
| 325 | 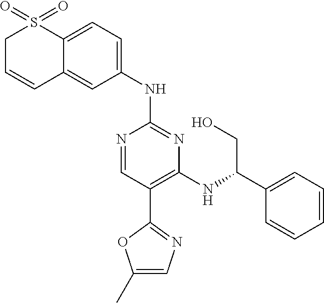 [SMILES: Cc1cnc(-c2cnc(Nc3ccc4c(c3)C=CCS4(=O)=O)nc2N[C@H](CO)c2ccccc2)o1] | (2S)-2-[[2-[(1,1-dioxo-3,4-dihydro- 2H-thiochromen-6-yl)amino]-5-(5- methyloxazol-2-yl)pyrimidin-4- yl]amino]-2-phenyl-ethanol | 492.2 | 492 |
| 326 | 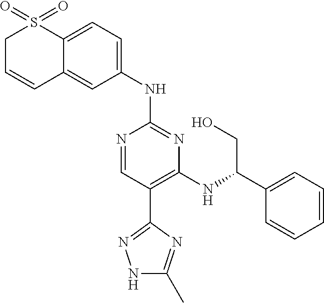 [SMILES: Cc1nc(-c2cnc(Nc3ccc4c(c3)C=CCS4(=O)=O)nc2N[C@H](CO)c2ccccc2)n[nH]1] | (2S)-2-[[2-[(1,1-dioxo-3,4-dihydro- 2H-thiochromen-6-yl)amino]-5-(5- methyl-1H-1,2,4-triazol-3- yl)pyrimdin-4-yl]amino]-2-phenyl- ethanol | 492.2 | 942 |
| 327 | 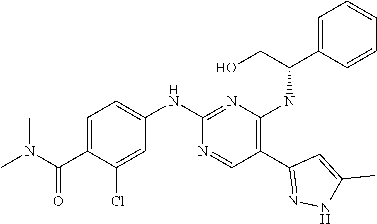 | 2-chloro-4-[[4-[[(1S)-2-hydroxy-1- phenyl-ethyl]amino]-5-(5-methyl-1H- pyrazol-3-yl)pyrimidin-2-yl]amino]- N,N-dimethyl-benzamide | 492.2 | 492 |
| 328 | 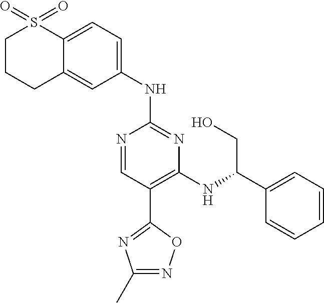 [SMILES: Cc1noc(-c2cnc(Nc3ccc4c(c3)CCCS4(=O)=O)nc2N[C@H](CO)c2ccccc2)n1] | (2S)-2-[[2-[(1,1-dioxo-3,4-dihydro- 2H-thiochromen-6-yl)amino]-5-(3- methyl-1,2,4-oxadiazol-5- yl)pyrimidin-4-yl]amino]-2-phenyl- ethanol | 493.2 | 493 |
| 329 | 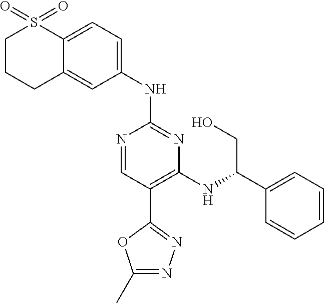 [SMILES: Cc1nnc(-c2cnc(Nc3ccc4c(c3)CCCS4(=O)=O)nc2N[C@H](CO)c2ccccc2)o1] | (2S)-2-[[2-[(1,1-dioxo-3,4-dihydro- 2H-thiochromen-6-yl)amino]-5-(5- methyl-1,3,4-oxadiazol-2- yl)pyrimidin-4-yl]amino]-2-phenyl- ethanol | 493.2 | 493 |
| 330 | 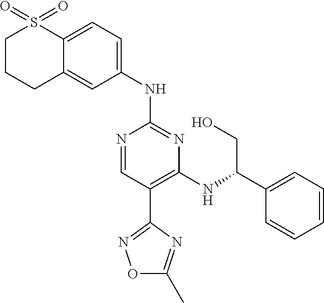 [SMILES: Cc1nc(-c2cnc(Nc3ccc4c(c3)CCCS4(=O)=O)nc2N[C@H](CO)c2ccccc2)no1] | (2S)-2-[[2-[(1,1-dioxo-3,4-dihydro- 2H-thiochromen-6-yl)amino]-5-(5- methyl-1,2,4-oxadiazol-3- yl)pyrimidin-4-yl]amino]-2-phenyl- ethanol | 493.2 | 493 |
| 331 | 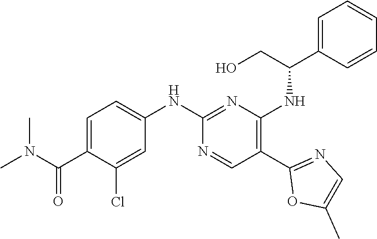 [SMILES: Cc1cnc(-c2cnc(Nc3ccc(C(=O)N(C)C)c(Cl)c3)nc2N[C@H](CO)c2ccccc2)o1] | 2-chloro-4-[[4-[[(1S)-2-hydroxy-1- phenyl-ethyl]amino]-5-(5- methyloxazol-2-yl)pyrimidin-2- yl]amino]-N,N-dimethyl-benzamide | 493.2 | 493 |
| 332 | 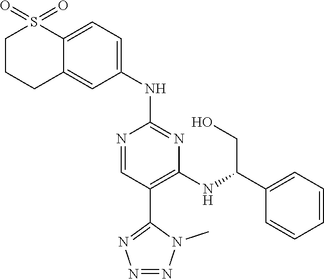 [SMILES: Cn1nnnc1-c1cnc(Nc2ccc3c(c2)CCCS3(=O)=O)nc1N[C@H](CO)c1ccccc1] | (2S)-2-[[2-[(1,1-dioxo-3,4-dihydro- 2H-thiochromen-6-yl)amino]-5-(1- methyltetrazol-5-yl)pyrimidin-4- yl]amino]-2-phenyl-ethanol | 493.2 | 493 |
| 333 | 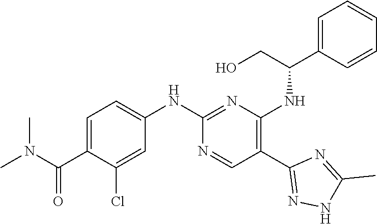 [SMILES: Cc1nc(-c2cnc(Nc3ccc(C(=O)N(C)C)c(Cl)c3)nc2N[C@H](CO)c2ccccc2)n[nH]1] | 2-chloro-4-[[4-[[(1S)-2-hydroxy-1- phenyl-ethyl]amino]-5-(5-methyl-1H- 1,2,4-triazol-3-yl)pyrimidin-2- yl]amino]-N,N-dimethyl-benzamide | 493.2 | 493 |
| 334 | 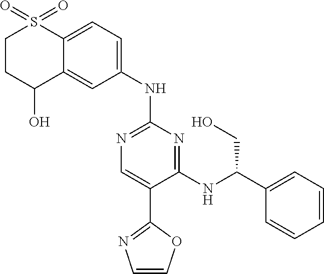 [SMILES: O=S1(=O)CCC(O)c2cc(Nc3ncc(-c4ncco4)c(N[C@H](CO)c4ccccc4)n3)ccc21] | 6-[[4-[[(1S)-2-hydroxy-1-phenyl- ethyl]amino]-5-oxazol-2-yl- pyrimidin-2-yl]amino]-1,1-dioxo-3,4- dihydro-2H-thiochromen-4-ol | 494.2 | 494 |
| 335 | 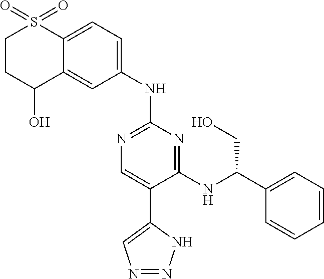 [SMILES: O=S1(=O)CCC(O)c2cc(Nc3ncc(-c4cnn[nH]4)c(N[C@H](CO)c4ccccc4)n3)ccc21] | 6-[[4-[[(1S)-2-hydroxy-1-phenyl- ethyl]amino]-5-(1H-triazol-5- yl)pyrimidin-2-yl]amino]-1,1-dioxo- 3,4-dihydro-2H-thiochromen-4-ol | 494.2 | 494 |
| 336 | 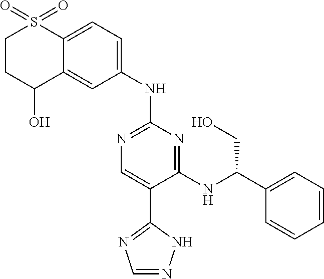 [SMILES: O=S1(=O)CCC(O)c2cc(Nc3ncc(-c4ncn[nH]4)c(N[C@H](CO)c4ccccc4)n3)ccc21] | 6-[[4-[[(1S)-2-hydroxy-1-phenyl- ethyl]amino]-5-(1H-1,2,4-triazol-5- yl)pyrimidin-2-yl]amino]-1,1-dioxo- 3,4-dihydro-2H-thiochromen-4-ol | 494.2 | 494 |
| 337 | 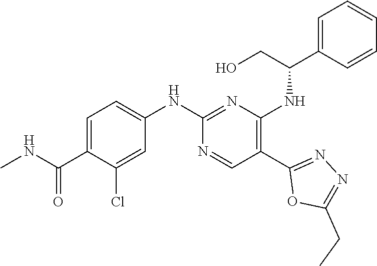 [SMILES: CCc1nnc(-c2cnc(Nc3ccc(C(=O)NC)c(Cl)c3)nc2N[C@H](CO)c2ccccc2)o1] | 2-chloro-4-[[5-(5-ethyl-1,3,4- oxadiazol-2-yl)-4-[[(1S)-2-hydroxy-1- phenyl-ethyl]amino]pyrimidin-2- yl]amino]-N-methyl-benzamide | 494.2 | 494 |
| 338 | 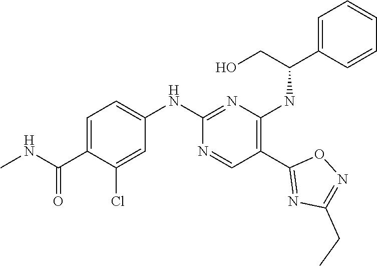 | 2-chloro-4-[[5-(3-ethyl-1,2,4- oxadiazol-5-yl)-4-[[(1S)-2-hydroxy-1- phenyl-ethyl]amino]pyrimidin-2- yl]amino]-N-methyl-benzamide | 494.2 | 494 |
| 339 | 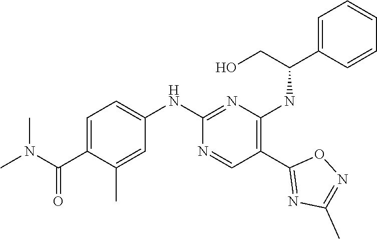 | 2-chloro-4-[[4-[[(1S)-2-hydroxy-1- phenyl-ethyl]amino]-5-(3-methyl- 1,2,4-oxadiazol-5-yl)pyrimidin-2- yl]amino]-N,N-dimethyl-benzamide | 494.2 | 494 |
| 340 | 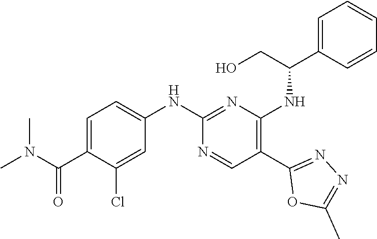 [SMILES: Cc1nnc(-c2cnc(Nc3ccc(C(=O)N(C)C)c(Cl)c3)nc2N[C@H](CO)c2ccccc2)o1] | 2-chloro-4-[[4-[[(1S)-2-hydroxy-1- phenyl-ethyl]amino]-5-(5-methyl- 1,3,4-oxadiazol-3-yl)pyrimidin-2- yl]amino]-N,N-dimethyl-benzamide | 494.2 | 494 |
| 341 | 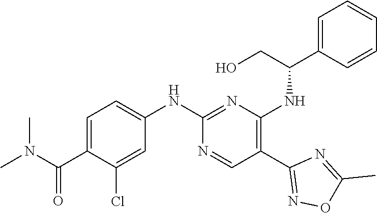 [SMILES: Cc1nc(-c2cnc(Nc3ccc(C(=O)N(C)C)c(Cl)c3)nc2N[C@H](CO)c2ccccc2)no1] | 2-chloro-4-[[4-[[(1S)-1-hydroxy-1- phenyl-ethyl]amino]-5-(5-methyl- 1,2,4-oxadiazol-3-yl)pyrimidin-2- yl]amino]-N,N-dimethyl-benzamide | 494.2 | 494 |
| 342 | 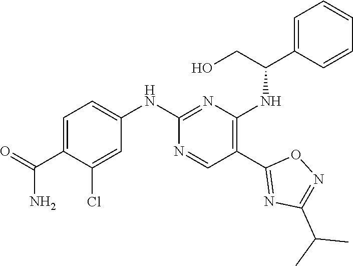 [SMILES: CC(C)c1noc(-c2cnc(Nc3ccc(C(N)=O)c(Cl)c3)nc2N[C@H](CO)c2ccccc2)n1] | 2-chloro-4-[[4-[[(1S)-2-hydroxy-1- phenyl-ethyl]amino]-5-(3-isopropyl- 1,2,4-oxadiazol-5-yl)pyrimidin-2- yl]amino]benzamide | 494.2 | 494 |
| 343 | 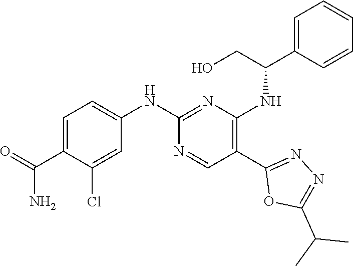 [SMILES: CC(C)c1nnc(-c2cnc(Nc3ccc(C(N)=O)c(Cl)c3)nc2N[C@H](CO)c2ccccc2)o1] | 2-chloro-4-[[4-[[(1S)-2-hydroxy-1- phenyl-ethyl]amino]-5-(5-isopropyl- 1,3,4-oxadiazol-2-yl)pyrimidin-2- yl]amino]benzamide | 494.2 | 494 |
| 344 | 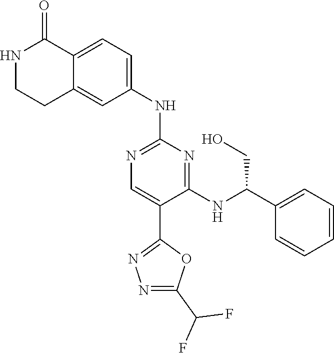 [SMILES: O=C1NCCc2cc(Nc3ncc(-c4nnc(C(F)F)o4)c(N[C@H](CO)c4ccccc4)n3)ccc21] | 6-[[5-[5-(difluoromethyl)-1,3,4- oxadiazol-2-yl]-4-[[(1S)-2-hydroxy-1- phenyl-ethyl]amino]pyrimidin-2- yl]amino]-3,4-dihydro-2H- isoquinolin-1-one | 494.2 | 494 |
| 345 | 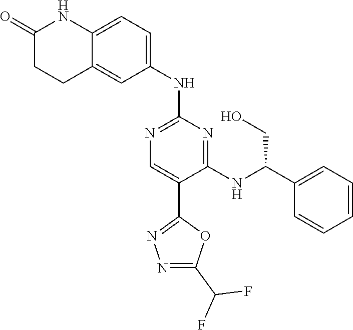 [SMILES: O=C1CCc2cc(Nc3ncc(-c4nnc(C(F)F)o4)c(N[C@H](CO)c4ccccc4)n3)ccc2N1] | 6-[[5-[5-(difluoromethyl)-1,3,4- oxadiazol-2-yl]-4-[[(1S)-2-hydroxy-1- phenyl-ethyl]amino]pyrimidin-2- yl]amino]-3,4-dihydro-1H-quinolin-2- one | 494.2 | 494 |
| 346 | 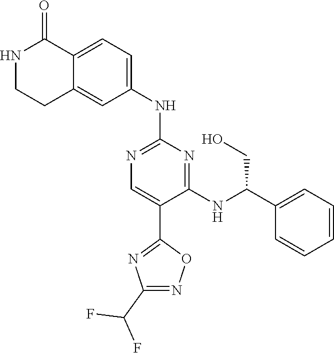 [SMILES: O=C1NCCc2cc(Nc3ncc(-c4nc(C(F)F)no4)c(N[C@H](CO)c4ccccc4)n3)ccc21] | 6-[[5-[3-(difluoromethyl)-1,2,4- oxadiazol-5-yl]-4-[[(1S)-2-hydroxy-1- phenyl-ethyl]amino]pyrimidin-2- yl]amino]-3,4-dihydro-2H- isoquinolin-1-one | 494.2 | 494 |
| 347 | 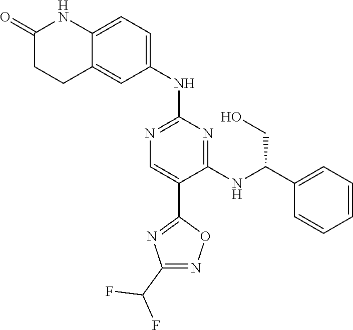 [SMILES: O=C1CCc2cc(Nc3ncc(-c4nc(C(F)F)no4)c(N[C@H](CO)c4ccccc4)n3)ccc2N1] | 6-[[5-[3-(difluoromethyl)-1,2,4- oxadiazol-5-yl]-4-[[(1S)-2-hydroxy-1- phenyl-ethyl]amino]pyrimidin-2- yl]amino]-3,4-dihydro-1H-quinolin-2- one | 494.2 | 494 |
| 348 | 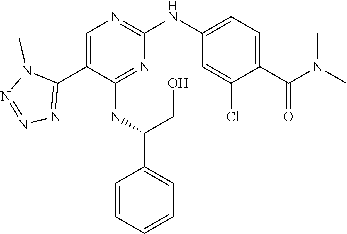 | 2-chloro-4-[[4-[[(1S)-2-hydroxy-1- phenyl-ethyl]amino]-5-(1- methyltetrazol-5-yl)pyrimidin-2- yl]amino]-N,N-dimethyl-benzamide | 494.2 | 494 |
| 349 | 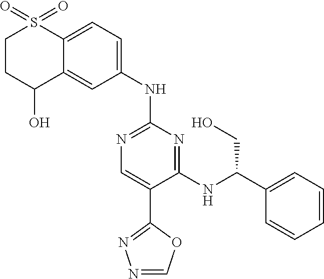 [SMILES: O=S1(=O)CCC(O)c2cc(Nc3ncc(-c4nnco4)c(N[C@H](CO)c4ccccc4)n3)ccc21] | 6-[[4-[[(1S)-2-hydroxy-1-phenyl- ethyl]amino]-5-(1,3,4-oxadiazol-2- yl)pyrimidin-2-yl]amino]-1,1-dioxo- 3,4-dihydro-2H-thiochromen-4-ol | 495.1 | 495 |
| 350 | 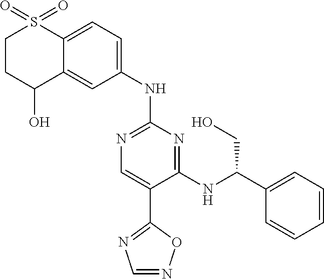 [SMILES: O=S1(=O)CCC(O)c2cc(Nc3ncc(-c4ncno4)c(N[C@H](CO)c4ccccc4)n3)ccc21] | 6-[[4-[[(1S)-2-hydroxy-1-phenyl- ethyl]amino]-5-(1,2,4-oxadiazol-5- yl)pyrimidin-2-yl]amino]-1,1-dioxo- 3,4-dihydro-2H-thiochromen-4-ol | 495.1 | 495 |
| 351 | 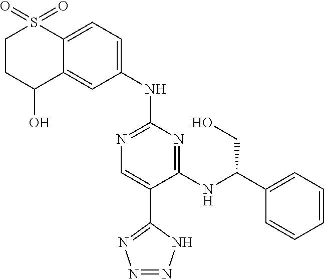 [SMILES: O=S1(=O)CCC(O)c2cc(Nc3ncc(-c4nnn[nH]4)c(N[C@H](CO)c4ccccc4)n3)ccc21] | 6-[[4-[[(1S)-2-hydroxy-1-phenyl- ethyl]amino]-5-(1H-tetrazol-5- yl)pyrimidin-2-yl]amino]-1,1-dioxo- 3,4-dihydro-2H-thiochromen-4-ol | 495.2 | 495 |
| 352 | 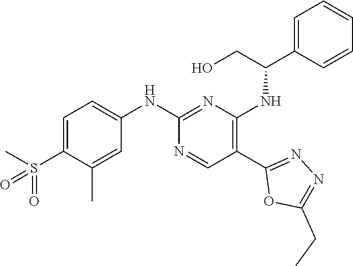 [SMILES: CCc1nnc(-c2cnc(Nc3ccc(S(C)(=O)=O)c(C)c3)nc2N[C@H](CO)c2ccccc2)o1] | (2S)-2-[[5-(5-ethyl-1,3,4-oxadiazol-2- yl)-2-(3-methyl-4-methylsulfonyl- anilino)pyrimidin-4-yl]amino]-2- phenyl-ethanol | 495.2 | 495 |
| 353 | 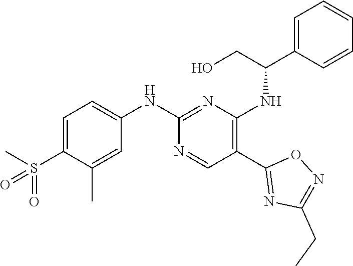 [SMILES: CCc1noc(-c2cnc(Nc3ccc(S(C)(=O)=O)c(C)c3)nc2N[C@H](CO)c2ccccc2)n1] | (2S)-2-[[5-(3-ethyl-1,2,4-oxadiazol-5- yl)-2-(3-methyl-4-methylsulfonyl- anilino)pyrimidin-4-yl]amino]-2- phenyl-ethanol | 495.2 | 495 |
| 354 | 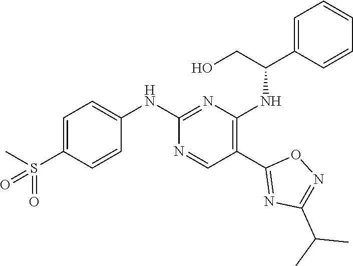 [SMILES: CC(C)c1noc(-c2cnc(Nc3ccc(S(C)(=O)=O)cc3)nc2N[C@H](CO)c2ccccc2)n1] | (2S)-2-[[5-(3-isopropyl-1,2,4- oxadiazol-5-yl)-2-(4- methylsulfonylanilino)-pyrimidin-4- yl]amino]-2-phenyl-ethanol | 495.2 | 495 |
| 355 | 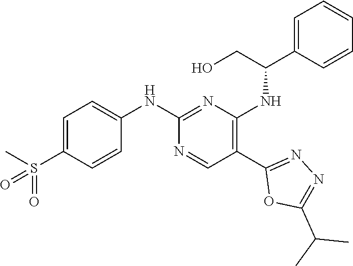 [SMILES: CC(C)c1nnc(-c2cnc(Nc3ccc(S(C)(=O)=O)cc3)nc2N[C@H](CO)c2ccccc2)o1] | (2S)-2-[[5-(5-isopropyl-1,3,4- oxadiazol-2-yl)-2-(4- methylsulfonylanilino)-pyrimidin-4- yl]amino]-2-phenyl-ethanol | 495.2 | 495 |
| 356 | 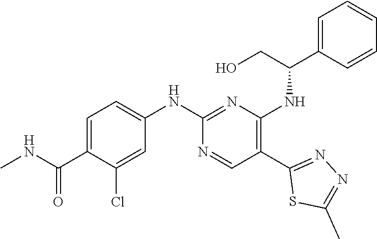 [SMILES: CNC(=O)c1ccc(Nc2ncc(-c3nnc(C)s3)c(N[C@H](CO)c3ccccc3)n2)cc1Cl] | 2-chloro-4-[[4-[[(1S)-2-hydroxy-1- phenyl-ethyl]amino]-5-(5-methyl- 1,3,4-thiadiazol-2-yl)pyrimidin-2- yl]amino]-N-methyl-benzamide | 496.1 | 496 |
| 357 | 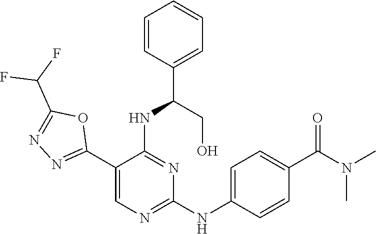 [SMILES: CN(C)C(=O)c1ccc(Nc2ncc(-c3nnc(C(F)F)o3)c(N[C@H](CO)c3ccccc3)n2)cc1] | 4-[[5-[5-(difluoromethyl)-1,3,4- oxadiazol-2-yl]-4-[[(1S)-2-hydroxy-1- phenyl-ethyl]amino]pyrimidin-2- yl]amino]-N,N-dimethyl-benzamide | 496.2 | 496 |
| 358 | 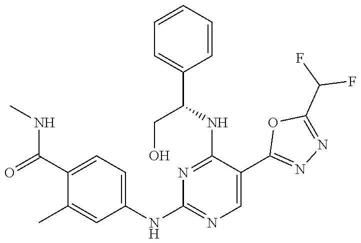 [SMILES: CNC(=O)c1ccc(Nc2ncc(-c3nnc(C(F)F)o3)c(N[C@H](CO)c3ccccc3)n2)cc1C] | 4-[[5-[5-(difluoromethyl)-1,3,4- oxadiazol-2-yl]-4-[[(1S)-2-hydroxy-1- phenyl-ethyl]amino]pyrimidin-2- yl]amino]-N,2-dimethyl-benzamide | 496.2 | 496 |
| 359 | 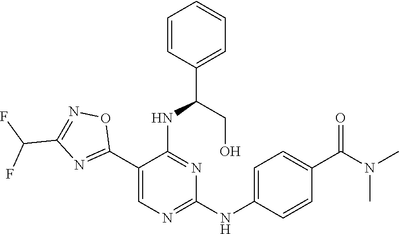 [SMILES: CN(C)C(=O)c1ccc(Nc2ncc(-c3nc(C(F)F)no3)c(N[C@H](CO)c3ccccc3)n2)cc1] | 4-[[5-[3-(difluoromethyl)-1,2,4- oxadiazol-5-yl]-4-[[(1S)-2-hydroxy-1- phenyl-ethyl]amino]pyrimidin-2- yl]amino]-N,N-dimethyl-benzamide | 496.2 | 496 |
| 360 | 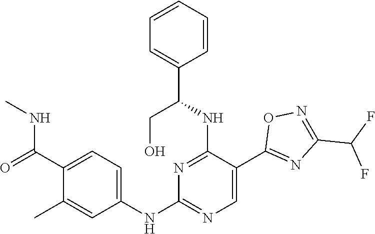 [SMILES: CNC(=O)c1ccc(Nc2ncc(-c3nc(C(F)F)no3)c(N[C@H](CO)c3ccccc3)n2)cc1C] | 4-[[5-[3-(difluoromethyl)-1,2,4- oxadiazol-5-yl]-4-[[(1S)-2-hydroxy-1- phenyl-ethyl]amino]pyrimidin-2- yl]amino]-N,2-dimethyl-benzamide | 496.2 | 496 |
| 361 | 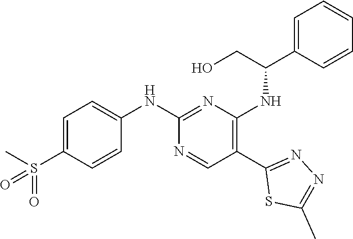 [SMILES: Cc1nnc(-c2cnc(Nc3ccc(S(C)(=O)=O)cc3)nc2N[C@H](CO)c2ccccc2)s1] | (2S)-2-[[2-(3-methyl-4- methylsulfonyl-anilino)-5-(5-methyl- 1,3,4-thiadiazol-2-yl)pyrimidin-4- yl]amino]-2-phenyl-ethanol | 497.1 | 497 |
| 362 | 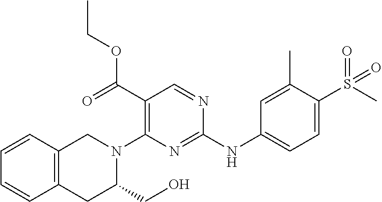 [SMILES: CCOC(=O)c1cnc(Nc2ccc(S(C)(=O)=O)c(C)c2)nc1N1Cc2ccccc2C[C@H]1CO] | ethyl 4-[(3S)-3-(hydroxymethyl)-3,4- dihydro-1H-isoquinolin-2-yl]-2-(3- methyl-4-methylsulfonyl- anilino)pyrimidine-5-carboxylate | 497.2 | 497 |
| 363 | 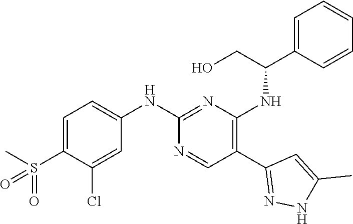 [SMILES: Cc1cc(-c2cnc(Nc3ccc(S(C)(=O)=O)c(Cl)c3)nc2N[C@H](CO)c2ccccc2)n[nH]1] | (2S)-2-[[2-(3-chloro-4- methylsulfonyl-anilino)-5-(5-methyl- 1H-pyrazol-3-yl)pyrimidin-4- yl]amino]-2-phenyl-ethanol | 499.1 | 499 |
| 364 | 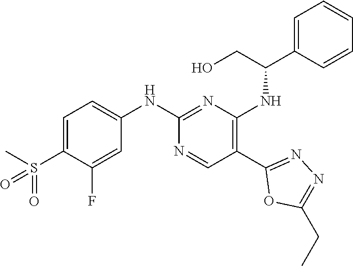 [SMILES: CCc1nnc(-c2cnc(Nc3ccc(S(C)(=O)=O)c(F)c3)nc2N[C@H](CO)c2ccccc2)o1] | (2S)-2-[[5-(5-ethyl-1,3,4-oxadiazol-2- yl)-2-(3-fluoro-4-methylsulfonyl- anilino)pyrimidin-4-yl]amino]-2- phenyl-ethanol | 499.2 | 499 |
| 365 | 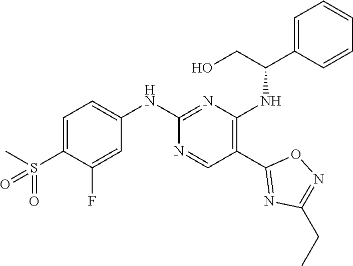 [SMILES: CCc1noc(-c2cnc(Nc3ccc(S(C)(=O)=O)c(F)c3)nc2N[C@H](CO)c2ccccc2)n1] | (2S)-2-[[5-(3-ethyl-1,2,4-oxadiazol-5- yl)-2-(3-fluoro-4-methylsulfonyl- anilino)pyrimidin-4-yl]amino]-2- phenyl-ethanol | 499.2 | 499 |
| 366 | 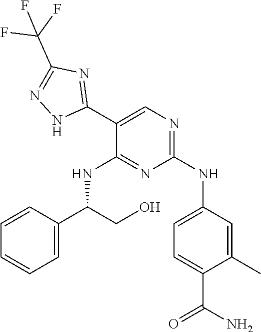 [SMILES: Cc1cc(Nc2ncc(-c3nc(C(F)(F)F)n[nH]3)c(N[C@H](CO)c3ccccc3)n2)ccc1C(N)=O] | 4-[[4-[[(1S)-2-hydroxy-1-phenyl- ethyl]amino]-5-(3-(trifluoromethyl)- 1H-1,2,4-triazol-5-yl]pyrimidin-2- yl]amino]-2-methyl-benzamide | 499.2 | 499 |
| 367 | 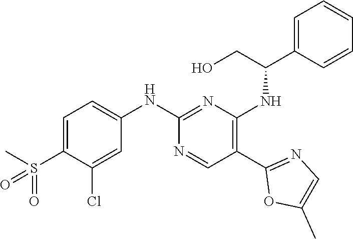 [SMILES: Cc1cnc(-c2cnc(Nc3ccc(S(C)(=O)=O)c(Cl)c3)nc2N[C@H](CO)c2ccccc2)o1] | (2S)-2-[[2-(3-chloro-4- methylsulfonyl-anilino)-5-(5- methyloxazol-2-yl)pyrimidin-4- yl]amino]-2-phenyl-ethanol | 500.1 | 500 |
| 368 | 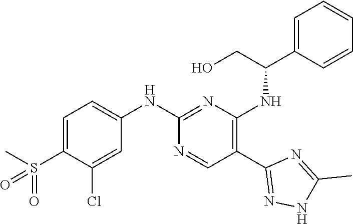 [SMILES: Cc1nc(-c2cnc(Nc3ccc(S(C)(=O)=O)c(Cl)c3)nc2N[C@H](CO)c2ccccc2)n[nH]1] | (2S)-2-[[2-(3-chloro-4- methylsulfonyl-anilino)-5-(5-methyl- 1H-1,2,4-triazol-3-yl)pyrimidin-4- yl]amino]-2-phenyl-ethanol | 500.1 | 500 |
| 369 | 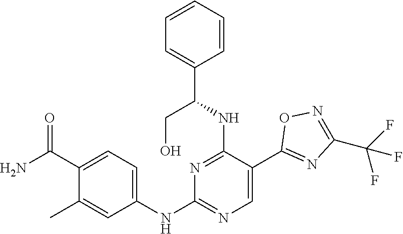 [SMILES: Cc1cc(Nc2ncc(-c3nc(C(F)(F)F)no3)c(N[C@H](CO)c3ccccc3)n2)ccc1C(N)=O] | 4-[[4-[[(1S)-2-hydroxy-1-phenyl- ethyl]amino]-5-[3-(trifluoromethyl)- 1,2,4-oxadiazol-5-yl]pyrimidin-2- yl]amino]-2-methyl-benzamide | 500.2 | 500 |
| 370 | 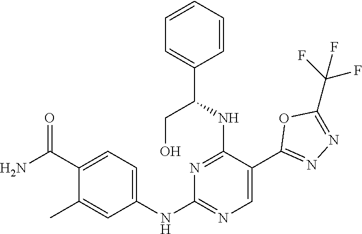 [SMILES: Cc1cc(Nc2ncc(-c3nnc(C(F)(F)F)o3)c(N[C@H](CO)c3ccccc3)n2)ccc1C(N)=O] | 4-[[4-[[(1S)-2-hydroxy-1-phenyl- ethyl]amino]-5-[5-(trifluoromethyl)- 1,3,4-oxadiazol-2-yl]pyrimidin-2- yl]amino]-2-methyl-benzamide | 500.2 | 500 |
| 371 | 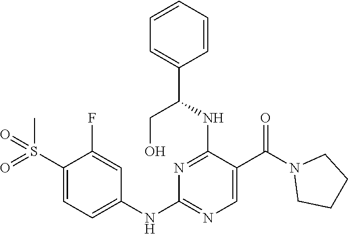 [SMILES: CS(=O)(=O)c1ccc(Nc2ncc(C(=O)N3CCCC3)c(N[C@H](CO)c3ccccc3)n2)cc1F] | [2-(3-fluoro-4-methylsulfonyl- anilino)-4-[[(1S)-2-hydroxy-1-phenyl- ethyl]amino]pyrimidin-5-yl]- pyrrolidin-1-yl-methanone | 500.2 | 500 |
| 372 | 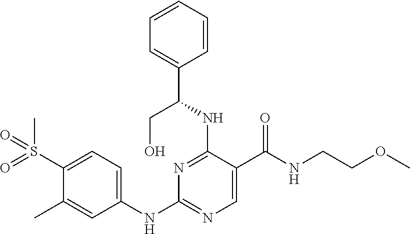 [SMILES: COCCNC(=O)c1cnc(Nc2ccc(S(C)(=O)=O)c(C)c2)nc1N[C@H](CO)c1ccccc1] | 4-[[(1S)-2-hydroxy-1-phenyl- ethyl]amino]-N-(2-methoxyethyl)-2- (3-methyl-4-methylsulfonyl- anilino)pyrimidin-5-carboxamide | 500.2 | 500 |
| 373 | 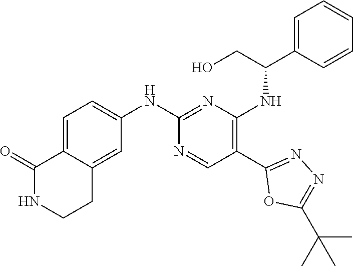 [SMILES: CC(C)(C)c1nnc(-c2cnc(Nc3ccc4c(c3)CCNC4=O)nc2N[C@H](CO)c2ccccc2)o1] | 6-[[5-(5-tert-butyl-1,3,4-oxadiazol-2- yl)-4-[[(1S)-2-hydroxy-1-phenyl- ethyl]amino]-pyrimidin-2-yl]amino]- 3,4-dihydro-2H-isoquinolin-1-one | 500.2 | 500 |
| 374 | 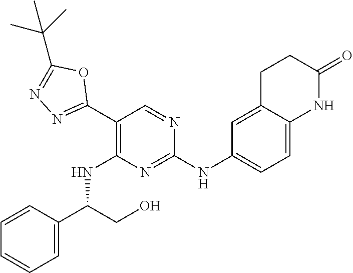 [SMILES: CC(C)(C)c1nnc(-c2cnc(Nc3ccc4c(c3)CCC(=O)N4)nc2N[C@H](CO)c2ccccc2)o1] | 6-[[5-(5-tert-butyl-1,3,4-oxadiazol-2- yl)-4-[[(1S)-2-hydroxy-1-phenyl- ethyl]amino]pyrimidi-2-yl]amino]- 3,4-dihydro-1H-quinolin-2-one | 500.2 | 500 |
| 375 | 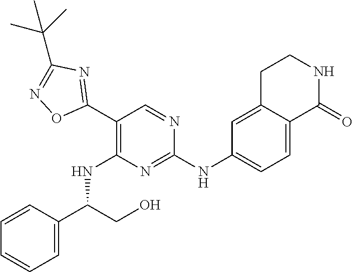 [SMILES: CC(C)(C)c1noc(-c2cnc(Nc3ccc4c(c3)CCNC4=O)nc2N[C@H](CO)c2ccccc2)n1] | 6-[[5-(3-tert-butyl-1,2,4-oxadiazol-5- yl)-4-[[(1S)-2-hydroxy-1-phenyl- ethyl]amino]pyrimidin-2-yl]amino]- 3,4-dihydro-2H-isoquinolin-1-one | 500.2 | 500 |
| 376 | 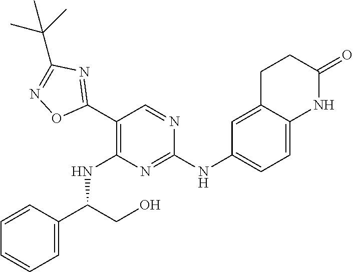 [SMILES: CC(C)(C)c1noc(-c2cnc(Nc3ccc4c(c3)CCC(=O)N4)nc2N[C@H](CO)c2ccccc2)n1] | 6-[[5-(3-tert-butyl-1,2,4-oxadiazol-5- yl)-4-[[(1S)-2-hydroxy-1-phenyl- ethyl]amino]pyrimidin-2-yl]amino]- 3,4-dihydro-1H-quinolin-2-one | 500.2 | 500 |
| 377 | 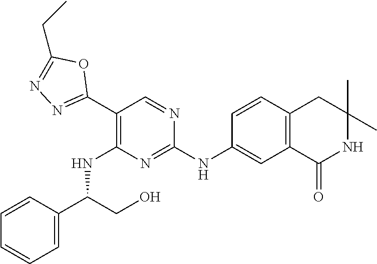 [SMILES: CCc1nnc(-c2cnc(Nc3ccc4c(c3)C(=O)NC(C)(C)C4)nc2N[C@H](CO)c2ccccc2)o1] | 7-[[5-(5-ethyl-1,3,4-oxadiazol-2-yl)- 4-[[(1S)-2-hydroxy-1-phenyl- ethyl]amino]pyrimidin-2-yl]amino]- 3,3-dimethyl-2,4-dihydroisoquinolin- 1-one | 500.2 | 500 |
| 378 | 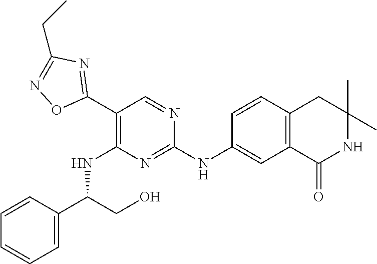 [SMILES: CCc1noc(-c2cnc(Nc3ccc4c(c3)C(=O)NC(C)(C)C4)nc2N[C@H](CO)c2ccccc2)n1] | 7-[[5-(3-ethyl-1,2,4-oxadiazol-5-yl)- 4-[[(1S)-2-hydroxy-1-phenyl- ethyl]amino]pyrimidin-2-yl]amino]- 3,3-dimethyl-2,4-dihydroisoquinolin- 1-one | 500.2 | 500 |
| 379 | 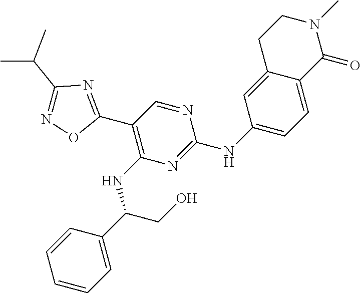 [SMILES: CC(C)c1noc(-c2cnc(Nc3ccc4c(c3)CCN(C)C4=O)nc2N[C@H](CO)c2ccccc2)n1] | 6-[[4-[[(1S)-2-hydroxy-1-phenyl- ethyl]amino]-5-(3-isopropyl-1,2,4- oxadiazol-5-yl)pyrimidin-2- yl]amino]-2-methyl-3,4- dihydroisoquinolin-1-one | 500.2 | 500 |
| 380 | 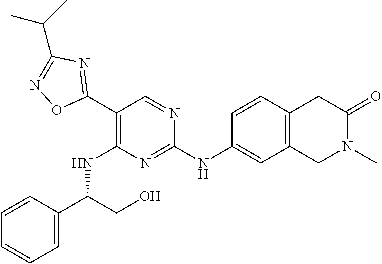 [SMILES: CC(C)c1noc(-c2cnc(Nc3ccc4c(c3)CN(C)C(=O)C4)nc2N[C@H](CO)c2ccccc2)n1] | 7-[[4-[[(1S)-2-hydroxy-1-phenyl- ethyl]amino]-5-(3-isopropyl-1,2,4- oxadiazol-5-yl)pyrimidin-2- yl]amino]-2-methyl-1,4- dihydroisoquinolin-3-one | 500.2 | 500 |
| 381 | 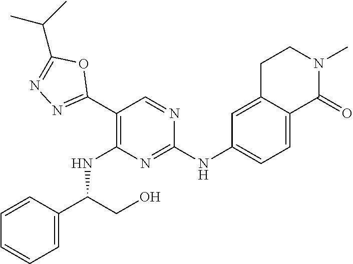 [SMILES: CC(C)c1nnc(-c2cnc(Nc3ccc4c(c3)CCN(C)C4=O)nc2N[C@H](CO)c2ccccc2)o1] | 6-[[4-[[(1S)-2-hydroxy-1-phenyl- ethyl]amino]-5-(5-isopropyl-1,3,4- oxadiazol-2-yl)pyrimidin-2- yl]amino]-2-methyl-3,4- dihydroisoquinolin-1-one | 500.5 | 500 |
| 382 | 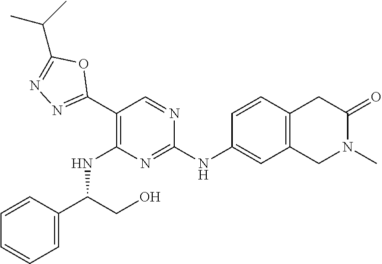 [SMILES: CC(C)c1nnc(-c2cnc(Nc3ccc4c(c3)CN(C)C(=O)C4)nc2N[C@H](CO)c2ccccc2)o1] | 7-[[4-[[(1S)-2-hydroxy-1-phenyl- ethyl]amino]-5-(5-isopropyl-1,3,4- oxadiazol-2-yl)pyrimidin-2- yl]amino]-2-methyl-1,4- dihydroisoquinolin-3-one | 500.2 | 500 |
| 383 | 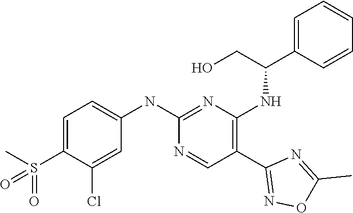 [SMILES: Cc1nc(-c2cnc(Nc3ccc(S(C)(=O)=O)c(Cl)c3)nc2N[C@H](CO)c2ccccc2)no1] | (2S)-2-[[2-(3-chloro-4- methylsulfonyl-anilino)-5-(5-methyl- 1,2,4-oxadiazol-3-yl)pyrimidin-4- yl]amino]-2-phenyl-ethanol | 501.1 | 501 |
| 384 | 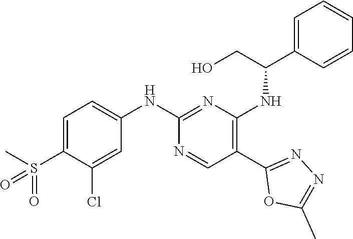 [SMILES: Cc1nnc(-c2cnc(Nc3ccc(S(C)(=O)=O)c(Cl)c3)nc2N[C@H](CO)c2ccccc2)o1] | (2S)-2-[[2-(3-chloro-4- methylsulfonyl-anilino)-5-(5-methyl- 1,3,4-oxadiazol-2-yl)pyrimidin-4- yl]amino]-2-phenyl-ethanol | 501.1 | 501 |
| 385 | 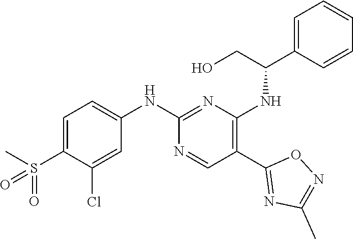 [SMILES: Cc1noc(-c2cnc(Nc3ccc(S(C)(=O)=O)c(Cl)c3)nc2N[C@H](CO)c2ccccc2)n1] | (2S)-2-[[2-(3-chloro-4- methylsulfonyl-anilino)-5-(3-methyl- 1,2,4-oxadiazol-5-yl)pyrimidin-4- yl]amino]-2-phenyl-ethanol | 501.1 | 501 |
| 386 | 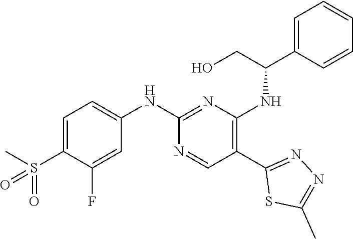 [SMILES: Cc1nnc(-c2cnc(Nc3ccc(S(C)(=O)=O)c(F)c3)nc2N[C@H](CO)c2ccccc2)s1] | (2S)-2-[[2-(3-fluroo-4- methylsulfonyl-anilino)-5-(5-methyl- 1,3,4-thiadiazol-2-yl)pyrimidin-4- yl]amino]-2-phenyl-ethanol | 501.1 | 501 |
| 387 | 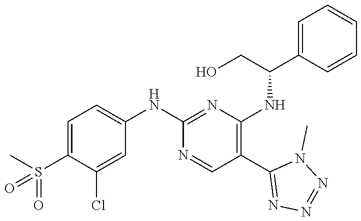 [SMILES: Cn1nnnc1-c1cnc(Nc2ccc(S(C)(=O)=O)c(Cl)c2)nc1N[C@H](CO)c1ccccc1] | (2S)-2-[[2-(3-chloro-4- methylsulfonyl-anilino)-5-(1- methyltetrazol-5-yl)pyrimidin-4- yl]amino]-2-phenyl-ethanol | 501.1 | 501 |
| 388 | 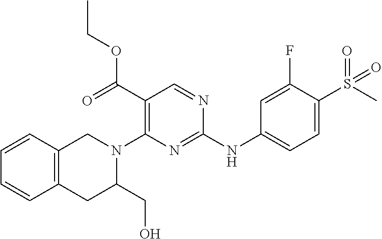 [SMILES: CCOC(=O)c1cnc(Nc2ccc(S(C)(=O)=O)c(F)c2)nc1N1Cc2ccccc2CC1CO] | ethyl 2-(3-fluoro-4-methylsulfonyl- anilino)-4-[3-(hydroxymethyl)-3,4- dihydro-1H-isoquinolin-2- yl]pyrimidine-5-carboxylate | 501.2 | 501 |
| 389 | 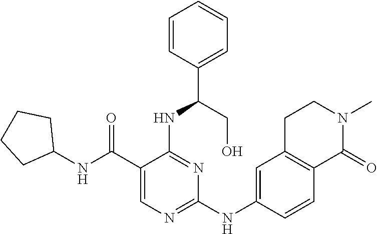 [SMILES: CN1CCc2cc(Nc3ncc(C(=O)NC4CCCC4)c(N[C@H](CO)c4ccccc4)n3)ccc2C1=O] | N-cyclopentyl-4-[[(1S)-2-hydroxy-1- phenyl-ethyl]amino]-2-[(2-methyl-1- oxo-3,4-dihydroisoquinolin-6- yl)amino]pyrimidine-5-carboxmaide | 501.3 | 501 |
| 390 | 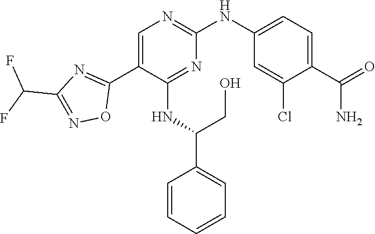 [SMILES: NC(=O)c1ccc(Nc2ncc(-c3nc(C(F)F)no3)c(N[C@H](CO)c3ccccc3)n2)cc1Cl] | 2-chloro-4-[[5-[3-(difluoromethyl)- 1,2,4-oxadiazol-5-yl]-4-[[(1S)-2- hydroxy-1-phenyl- ethyl]amino]pyrimidin-2- yl]amino]benzamide | 502.1 | 502 |
| 391 | 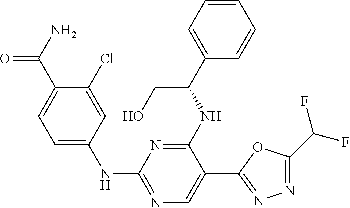 [SMILES: NC(=O)c1ccc(Nc2ncc(-c3nnc(C(F)F)o3)c(N[C@H](CO)c3ccccc3)n2)cc1Cl] | 2-chloro-4-[[5-[5-(difluoromethyl)- 1,3,4-oxadiazol-2-yl]-4-[[(1S)-2- hydroxy-1-phenyl- ethyl]amino]pyrimidin-2- yl]amino]benzamide | 502.1 | 502 |
| 392 | 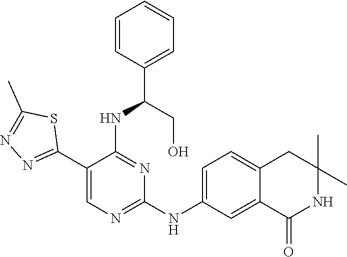 [SMILES: Cc1nnc(-c2cnc(Nc3ccc4c(c3)C(=O)NC(C)(C)C4)nc2N[C@H](CO)c2ccccc2)s1] | 7-[[4-[[(1S)-2-hydroxy-1-phenyl- ethyl]amino]-5-(5-methyl-1,3,4- thiadiazol-2-yl)pyrimidin-2- yl]amino]-3,3-dimethyl-2,4- dihydroisoquinolin-1-one | 502.2 | 502 |
| 393 | 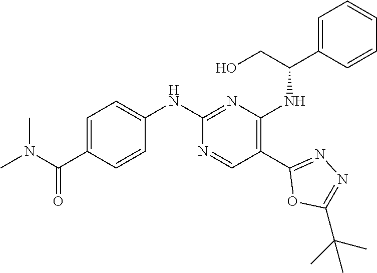 [SMILES: CN(C)C(=O)c1ccc(Nc2ncc(-c3nnc(C(C)(C)C)o3)c(N[C@H](CO)c3ccccc3)n2)cc1] | 4-[[5-(5-tert-butyl-1,3,4-oxadiazol-2- yl)-4-[[(1S)-2-hydroxy-1-phenyl- ethyl]amino]pyrimidin-2-yl]amino]- N,N-dimethyl-benzamide | 502.3 | 502 |
| 394 | 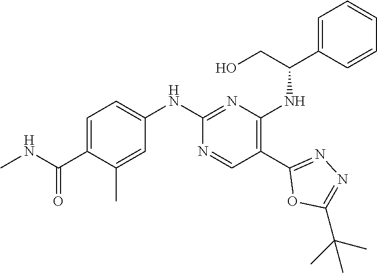 [SMILES: CNC(=O)c1ccc(Nc2ncc(-c3nnc(C(C)(C)C)o3)c(N[C@H](CO)c3ccccc3)n2)cc1C] | 4-[[5-(5-tert-butyl-1,3,4-oxadiazol-2- yl)-4-[[(1S)-2-hydroxy-1-phenyl- ethyl]amino]pyrimidin-2-yl]amino]- N,2-dimethyl-benzamide | 502.3 | 502 |
| 395 | 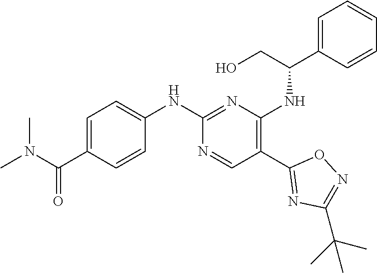 [SMILES: CN(C)C(=O)c1ccc(Nc2ncc(-c3nc(C(C)(C)C)no3)c(N[C@H](CO)c3ccccc3)n2)cc1] | 4-[[5-(3-tert-butyl-1,2,4-oxadiazol-5- yl)-4-[[(1S)-2-hydroxy-1-phenyl- ethyl]amino]pyrimidin-2-yl]amino]- N,N-dimethyl-benzamide | 502.3 | 502 |
| 396 | 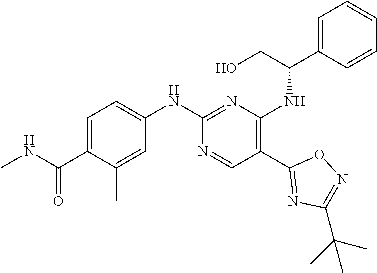 [SMILES: CNC(=O)c1ccc(Nc2ncc(-c3nc(C(C)(C)C)no3)c(N[C@H](CO)c3ccccc3)n2)cc1C] | 4-[[5-(3-tert-butyl-1,2,4-oxadiazol-5- yl)-4-[[(1S)-2-hydroxy-1-phenyl- ethyl]amino]pyrimidin-2-yl]amino]- N,2-dimethyl-benzamide | 502.3 | 502 |
| 397 | 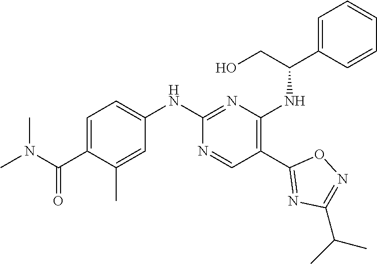 [SMILES: Cc1cc(Nc2ncc(-c3nc(C(C)C)no3)c(N[C@H](CO)c3ccccc3)n2)ccc1C(=O)N(C)C] | 4-[[4-[[(1S)-2-hydroxy-1-phenyl- ethyl]amino]-5-(3-isopropyl-1,2,4- oxadiazol-5-yl)pyrimidin-2- yl]amino]-N,N,2-trimethyl-benzamide | 502.3 | 502 |
| 398 | 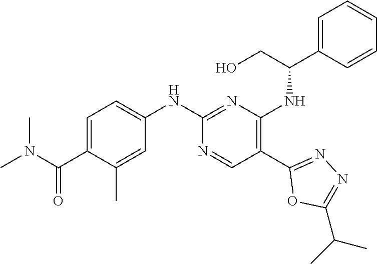 [SMILES: Cc1cc(Nc2ncc(-c3nnc(C(C)C)o3)c(N[C@H](CO)c3ccccc3)n2)ccc1C(=O)N(C)C] | 4-[[4-[[(1S)-2-hydroxy-1-phenyl- ethyl]amino]-5-(5-isopropyl-1,3,4- oxadiazol-2-yl)pyrimidin-2- yl]amino]-N,N,2-trimethyl-benzamide | 502.3 | 502 |
| 399 | 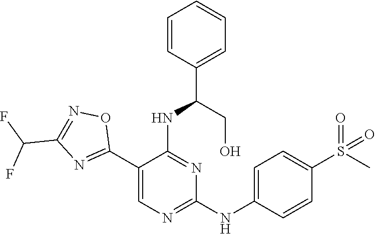 [SMILES: CS(=O)(=O)c1ccc(Nc2ncc(-c3nc(C(F)F)no3)c(N[C@H](CO)c3ccccc3)n2)cc1] | (2S)-2-[[5-[3-(difluoromethyl)-1,2,4- oxadiazol-5-yl]-2-(4- methylsulfonylanilino)-pyrimidin-4- yl]amino]-2-phenyl-ethanol | 503.1 | 503 |
| 400 | 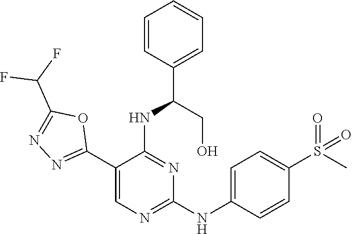 [SMILES: CS(=O)(=O)c1ccc(Nc2ncc(-c3nnc(C(F)F)o3)c(N[C@H](CO)c3ccccc3)n2)cc1] | (2S)-2-[[5-[5-(difluoromethyl)-1,3,4- oxadiazol-2-yl]-2-(4- methylsulfonylanilino)-pyrimidin-4- yl]amino]-2-phenyl-ethanol | 503.1 | 503 |
| 401 | 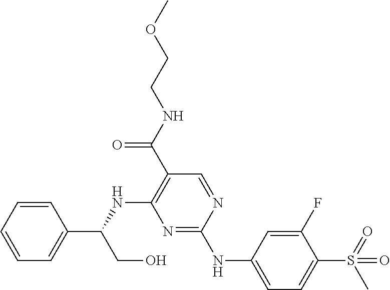 [SMILES: COCCNC(=O)c1cnc(Nc2ccc(S(C)(=O)=O)c(F)c2)nc1N[C@H](CO)c1ccccc1] | 2-(3-fluoro-4-methylsulfonyl- anilino)-4-[[(1S)-2-hydroxy-1-phenyl- ethyl]amino]-N-(2-methoxyethyl)- pyrimidine-5-carboxamide | 504.2 | 504 |
| 402 | 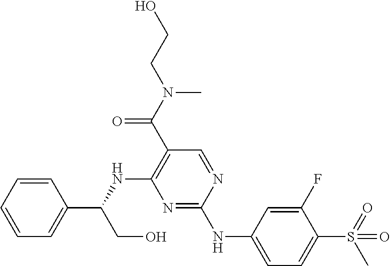 [SMILES: CN(CCO)C(=O)c1cnc(Nc2ccc(S(C)(=O)=O)c(F)c2)nc1N[C@H](CO)c1ccccc1] | 2-(3-fluoro-4-methylsuflonyl- anilino)-N-(2-hydroxyethyl)-4-[[(1S)- 2-hydroxy-1-phenyl-ethyl]amino]-N- methyl-pyrimidine-5-carboxamide | 504.2 | 504 |
| 403 | 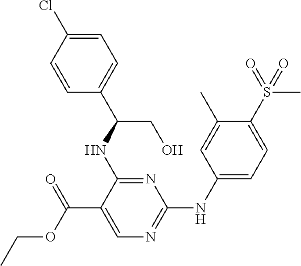 [SMILES: CCOC(=O)c1cnc(Nc2ccc(S(C)(=O)=O)c(C)c2)nc1N[C@H](CO)c1ccc(Cl)cc1] | ethyl 4-[[(1S)-1-(4-chlorophenyl)-2- hydroxy-ethyl]amino]-2-(3-methyl-4- methylsulfonyl-anilino)pyrimidine-5- carboxylate | 505.1 | 505 |
| 404 | 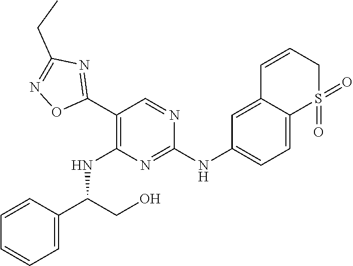 [SMILES: CCc1noc(-c2cnc(Nc3ccc4c(c3)C=CCS4(=O)=O)nc2N[C@H](CO)c2ccccc2)n1] | (2S)-2-[[2-[(1,1-dioxo-2H- thiochromen-6-yl)amino]-5-(3-ethyl- 1,2,4-oxadiazol-5-yl)pyrimidin-4- yl]amino]-2-phenyl-ethanol | 505.2 | 505 |
| 405 | 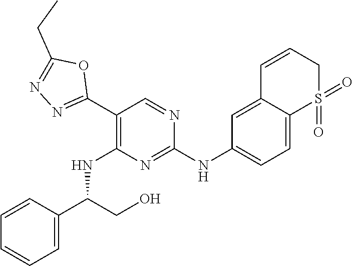 [SMILES: CCc1nnc(-c2cnc(Nc3ccc4c(c3)C=CCS4(=O)=O)nc2N[C@H](CO)c2ccccc2)o1] | (2S)-2-[[2-[(1,1-dioxo-2H- thiochromen-6-yl)amino]-5-(5-ethyl- 1,3,4-oxadiazol-2-yl)pyrimidin-4- yl]amino]-2-phenyl-ethanol | 505.2 | 505 |
| 406 | 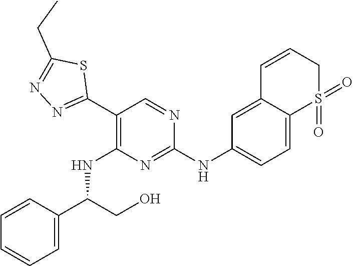 [SMILES: CCc1nnc(-c2cnc(Nc3ccc4c(c3)C=CCS4(=O)=O)nc2N[C@H](CO)c2ccccc2)s1] | (2S)-2-[[2-[(1,1-dioxo-2H- thiochromen-6-l)amino]-5-(5- methyl-1,3,4-thiadiazol-2- yl)pyrimidin-4-yl]amino]-2-phenyl- ethanol | 507.1 | 507 |
| 407 | 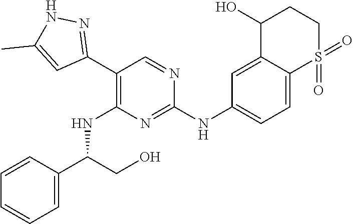 [SMILES: Cc1cc(-c2cnc(Nc3ccc4c(c3)C(O)CCS4(=O)=O)nc2N[C@H](CO)c2ccccc2)n[nH]1] | 6-[[4-[[(1S)-2-hydroxy-1-phenyl- ethyl]amino]-5-(5-methyl-1H- pyrazol-3-yl)pyrimidin-2-yl]amino]- 1,1-dioxo-3,4-dihydro-2H- thiochromen-4-ol | 507.2 | 507 |
| 408 | 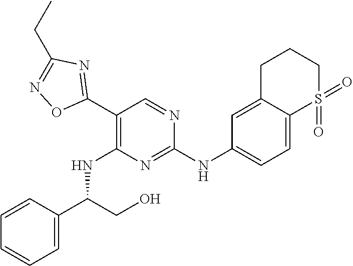 [SMILES: CCc1noc(-c2cnc(Nc3ccc4c(c3)CCCS4(=O)=O)nc2N[C@H](CO)c2ccccc2)n1] | (2S)-2-[[2-[(1,1-dioxo-3,4-dihydro- 2H-thiochromen-6-yl)amino]-5-(3- ethyl-1,2,4-oxadiazol-5-yl)pyrimidin- 4-yl]amino]-2-phenyl-ethanol | 507.2 | 507 |
| 409 | 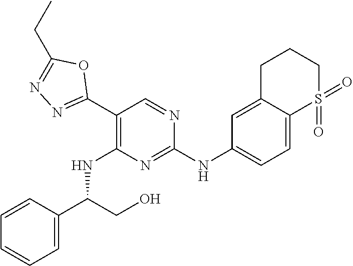 [SMILES: CCc1nnc(-c2cnc(Nc3ccc4c(c3)CCCS4(=O)=O)nc2N[C@H](CO)c2ccccc2)o1] | (2S)-2-[[2-[(1,1-dioxo-3,4-dihydro- 2H-thiochromen-6-yl)amino]-5-(5- ethyl-1,3,4-oxadiazol-2-yl)pyrimidin- 4-yl]amino]-2-phenyl-ethanol | 507.2 | 507 |
| 410 | 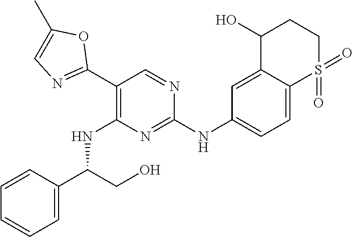 [SMILES: Cc1cnc(-c2cnc(Nc3ccc4c(c3)C(O)CCS4(=O)=O)nc2N[C@H](CO)c2ccccc2)o1] | 6-[[4-[[(1S)-2-hydroxy-1-phenyl- ethyl]amino]-5-(5-methyloxazol-2- yl)pyrimidin-2-yl]amino]-1,1-dioxo- 3,4-dihydro-2H-thiochromen-4-ol | 508.2 | 508 |
| 411 | 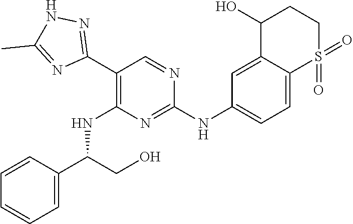 [SMILES: Cc1nc(-c2cnc(Nc3ccc4c(c3)C(O)CCS4(=O)=O)nc2N[C@H](CO)c2ccccc2)n[nH]1] | 6-[[4-[[(1S)-2-hydroxy-1-phenyl- ethyl]amino]-5-(5-methyl-1H-1,2,4- triazol-3-yl)pyrimidin-2-yl]amino]- 1,1-dioxo-3,4-dihydro-2H- thiochromen-4-ol | 508.2 | 508 |
| 412 | 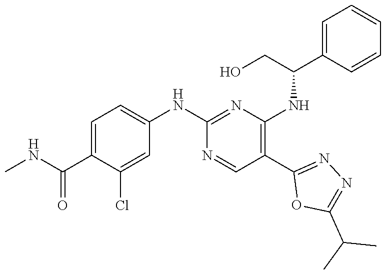 [SMILES: CNC(=O)c1ccc(Nc2ncc(-c3nnc(C(C)C)o3)c(N[C@H](CO)c3ccccc3)n2)cc1Cl] | 2-chloro-4-[[4-[[(1S)-2-hydroxy-1- phenyl-ethyl]amino]-5-(5-isopropyl- 1,3,4-oxadiazol-2-yl)pyrimidin-2- yl]amino]-N-methyl-benzamide | 508.2 | 508 |
| 413 | 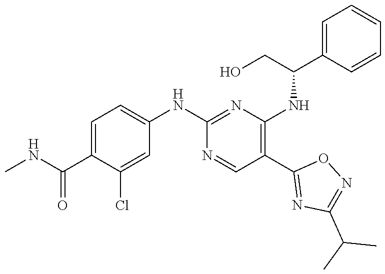 [SMILES: CNC(=O)c1ccc(Nc2ncc(-c3nc(C(C)C)no3)c(N[C@H](CO)c3ccccc3)n2)cc1Cl] | 2-chloro-4-[[4-[[(1S)-2-hydroxy-1- phenyl-ethyl]amino]-5-(3-isopropyl- 1,2,4-oxadiazol-5-yl]pyrimidin-2- yl]amino]-N-methyl-benzamide | 508.2 | 508 |
| 414 | 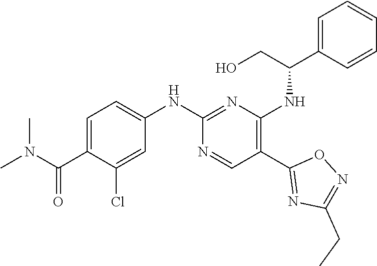 [SMILES: CCc1noc(-c2cnc(Nc3ccc(C(=O)N(C)C)c(Cl)c3)nc2N[C@H](CO)c2ccccc2)n1] | 2-chloro-4-[[5-(3-ethyl-1,2,4- oxadiazol-5-yl)-4-[[(1S)-2-hydroxy-1- phenyl-ethyl]amino]pyrimidin-2- yl]amino]-N,N-dimethyl-benzamide | 508.2 | 508 |
| 415 | 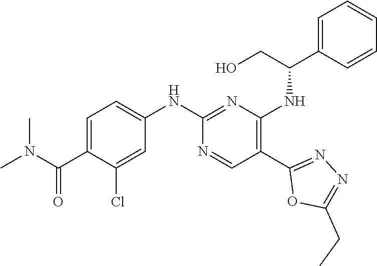 [SMILES: CCc1nnc(-c2cnc(Nc3ccc(C(=O)N(C)C)c(Cl)c3)nc2N[C@H](CO)c2ccccc2)o1] | 2-chloro-4-[[5-(5-ethyl-1,3,4- oxadiazol-2-yl)-4-[[(1S)-2-hydroxy-1- phenyl-ethyl]amino]pyrimidin-2- yl]amino]-N,N-dimethyl-benzamide | 508.2 | 508 |
| 416 | 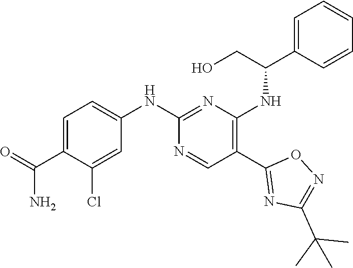 [SMILES: CC(C)(C)c1noc(-c2cnc(Nc3ccc(C(N)=O)c(Cl)c3)nc2N[C@H](CO)c2ccccc2)n1] | 4-[[5-(3-tert-butyl-1,2,4-oxadiazol-5- yl)-4-[[(1S)-2-hydroxy-1-phenyl- ethyl]amino]pyrimidin-2-yl]amino]-2- chloro-benzamide | 508.2 | 508 |
| 417 | 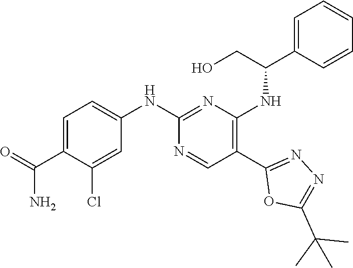 [SMILES: CC(C)(C)c1nnc(-c2cnc(Nc3ccc(C(N)=O)c(Cl)c3)nc2N[C@H](CO)c2ccccc2)o1] | 4-[[5-(5-tert-butyl-1,3,4-oxadiazol-2- yl)-4-[[(1S)-2-hydroxy-1-phenyl- ethyl]amino]pyrimidin-2-yl]amino]-2- chloro-benzamide | 508.2 | 508 |
| 418 | 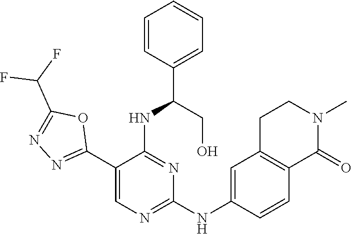 [SMILES: CN1CCc2cc(Nc3ncc(-c4nnc(C(F)F)o4)c(N[C@H](CO)c4ccccc4)n3)ccc2C1=O] | 6-[[5-[5-(difluoromethyl)-1,3,4- oxadiazol-2-yl]-4-[[(1S)-2-hydroxy-1- phenyl-ethyl]amino]pyrimidin-2- yl]amino]-2-methyl-3,4- dihydroisoquinolin-1-one | 508.2 | 508 |
| 419 | 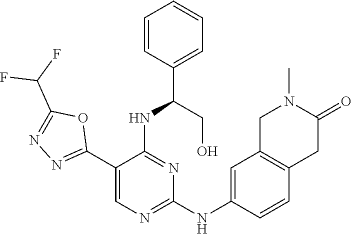 [SMILES: CN1Cc2cc(Nc3ncc(-c4nnc(C(F)F)o4)c(N[C@H](CO)c4ccccc4)n3)ccc2CC1=O] | 7-[[5-[5-(difluoromethyl)-1,3,4- oxadiazol-2-yl]-4-[[(1S)-2-hydroxy-1- phenyl-ethyl]amino]pyrimidi-2- yl]amino]-2-methyl-1,4- dihydroisoquinolin-3-one | 508.2 | 508 |
| 420 | 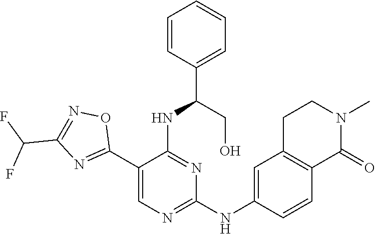 [SMILES: CN1CCc2cc(Nc3ncc(-c4nc(C(F)F)no4)c(N[C@H](CO)c4ccccc4)n3)ccc2C1=O] | 6-[[5-[3-(difluoromethyl)-1,2,4- oxadiazol-5-yl]-4-[[(1S)-2-hydroxy-1- phenyl-ethyl]amino]pyrimidin-2- yl]amio]-2-methyl-3,4- dihydroisoquinolin-1-one | 508.2 | 508 |
| 421 | 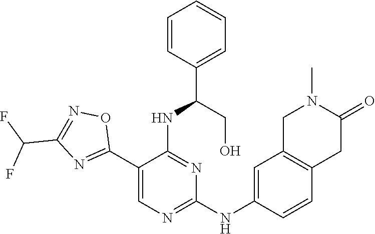 [SMILES: CN1Cc2cc(Nc3ncc(-c4nc(C(F)F)no4)c(N[C@H](CO)c4ccccc4)n3)ccc2CC1=O] | 7-[[5-[3-(difluoromethyl)-1,2,4- oxadiazol-5-yl]-4-[[(1S)-2-hydroxy-1- phenyl-ethyl]amino]pyrimidin-2- yl]amino]-2-methyl-1,4- dihydroisoquinolin-3-one | 508.2 | 508 |
| 422 | 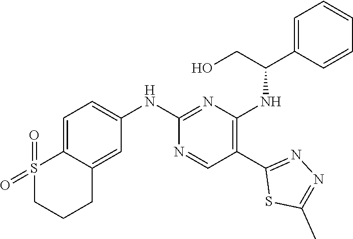 [SMILES: Cc1nnc(-c2cnc(Nc3ccc4c(c3)CCCS4(=O)=O)nc2N[C@H](CO)c2ccccc2)s1] | (2S)-2-[[2-[(1,1-dioxo-3,4-dihydro- 2H-thiochromen-6-yl)amino]-5-(5- methyl-1,3,4-thiadiazol-2- yl)pyrimidin-4-yl]amino]-2-phenyl- ethanol | 509.1 | 509 |
| 423 | 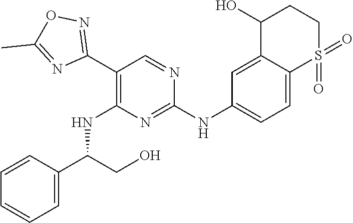 [SMILES: Cc1nc(-c2cnc(Nc3ccc4c(c3)C(O)CCS4(=O)=O)nc2N[C@H](CO)c2ccccc2)no1] | 6-[[4-[[(1S)-2-hydroxy-1-phenyl- ethyl]amino]-5-(5-methyl-1,2,4- oxadiazol-3-yl)pyrimidin-2- yl]amino]-1,1-dioxo-3,4-dihydro-2H- thiochromen-4-ol | 509.2 | 509 |
| 424 | 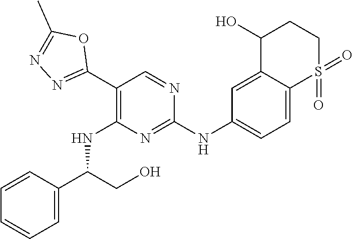 [SMILES: Cc1nnc(-c2cnc(Nc3ccc4c(c3)C(O)CCS4(=O)=O)nc2N[C@H](CO)c2ccccc2)o1] | 6-[[4-[[(1S)-2-hydroxy-1-phenyl- ethyl]amino]-5-(5-methyl-1,3,4- oxadiazol-2-yl)pyrimidin-2- yl]amino]-1,1-dioxo-3,4-dihydro-2H- thiochromen-4-ol | 509.2 | 509 |
| 425 | 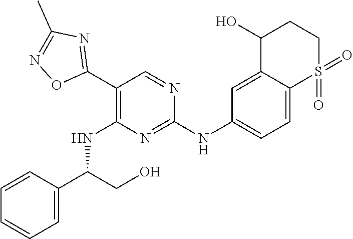 [SMILES: Cc1noc(-c2cnc(Nc3ccc4c(c3)C(O)CCS4(=O)=O)nc2N[C@H](CO)c2ccccc2)n1] | 6-[[4-[[(1S)-2-hydroxy-1-phenyl- ethyl]amino]-5-(3-methyl-1,2,4- oxadiazol-5-yl)pyrimidin-2- yl]amino]-1,1-dioxo-3,4-dihydro-2H- thiochromen-4-ol | 509.2 | 509 |
| 426 | 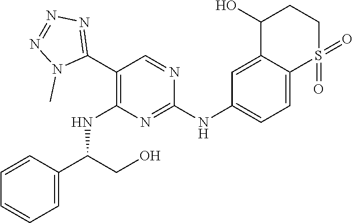 [SMILES: Cn1nnnc1-c1cnc(Nc2ccc3c(c2)C(O)CCS3(=O)=O)nc1N[C@H](CO)c1ccccc1] | 6-[[4-[[(1S)-2-hydroxy-1-phenyl- ethyl]amino]-5-(1-methyltetrazol-5- yl)pyrimidin-2-yl]amino]-1,1-dioxo- 3,4-dihydro-2H-thiochromen-4-ol | 509.2 | 509 |
| 427 | 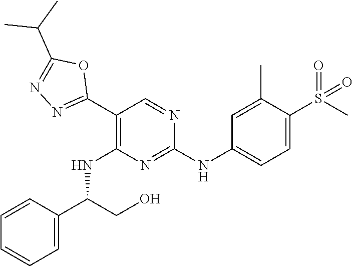 [SMILES: Cc1cc(Nc2ncc(-c3nnc(C(C)C)o3)c(N[C@H](CO)c3ccccc3)n2)ccc1S(C)(=O)=O] | (2S)-2-[[5-(5-isopropyl-1,3,4- oxadiazol-2-yl)-2-(3-methyl-4- methylsulfonyl-anilino)pyrimidin-4- yl]amino]-2-phenyl-ethanol | 509.2 | 509 |
| 428 | 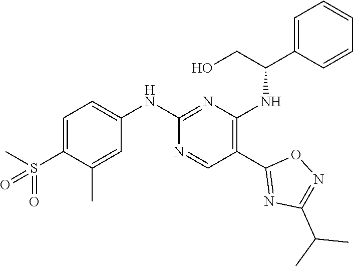 [SMILES: Cc1cc(Nc2ncc(-c3nc(C(C)C)no3)c(N[C@H](CO)c3ccccc3)n2)ccc1S(C)(=O)=O] | (2S)-2-[[5-(3-isopropyl-1,2,4- oxadiazol-5-yl)-2-(3-methyl-4- methylsulfonyl-anilino)pyrimidin-4- yl]amino]-2-phenyl-ethanol | 509.2 | 509 |
| 429 | 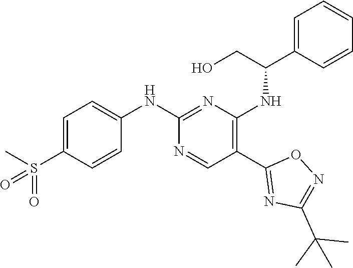 [SMILES: CC(C)(C)c1noc(-c2cnc(Nc3ccc(S(C)(=O)=O)cc3)nc2N[C@H](CO)c2ccccc2)n1] | (2S)-2-[[5-(3-tert-butyl-1,2,4- oxadiazol-5-yl)-2-(4- methylsulfonylanilino)-pyrimidin-4- yl]amino]-2-phenyl-ethanol | 509.2 | 509 |
| 430 | 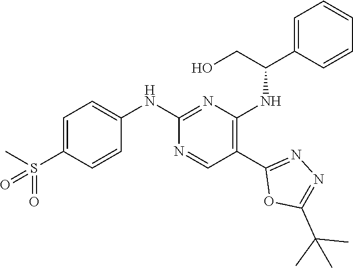 [SMILES: CC(C)(C)c1nnc(-c2cnc(Nc3ccc(S(C)(=O)=O)cc3)nc2N[C@H](CO)c2ccccc2)o1] | (2S)-2-[[5-(5-tert-butyl-1,3,4- oxadiazol-2-yl)-2-(4- methylsulfonylanilino)-pyrimidin-4- yl]amino]-2-phenyl-ethanol | 509.2 | 509 |
| 431 | 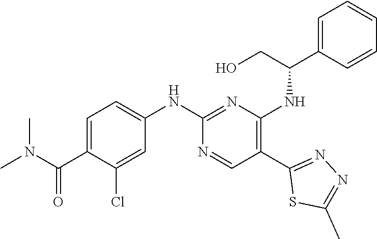 [SMILES: Cc1nnc(-c2cnc(Nc3ccc(C(=O)N(C)C)c(Cl)c3)nc2N[C@H](CO)c2ccccc2)s1] | 2-chloro-4-[[4-[[(1S)-2-hydroxy-1- phenyl-ethyl]amino]-5-(5-methyl- 1,3,4-thiadiazol-2-yl)pyrimidin-2- yl]amino]-N,N-dimethyl-benzamide | 510.1 | 510 |
| 432 | 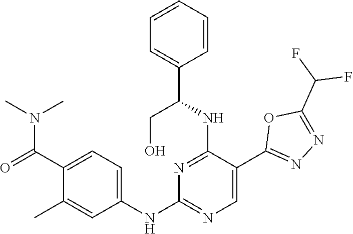 [SMILES: Cc1cc(Nc2ncc(-c3nnc(C(F)F)o3)c(N[C@H](CO)c3ccccc3)n2)ccc1C(=O)N(C)C] | 4-[[5-[5-(difluoromethyl)-1,3,4- oxadiazol-2-yl]-4-[[(1S)-2-hydroxy-1- phenyl-ethyl]amino]pyrimidin-2- yl]amino]-N,N,2-trimethyl-benzamide | 510.2 | 510 |
| 433 | 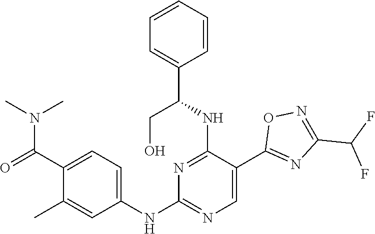 [SMILES: Cc1cc(Nc2ncc(-c3nc(C(F)F)no3)c(N[C@H](CO)c3ccccc3)n2)ccc1C(=O)N(C)C] | 4-[[5-[3-(difluoromethyl)-1,2,4- oxadiazol-5-yl]-4-[[(1S)-2-hydroxy-1- phenyl-ethyl]amino]pyrimidin-2- yl]amino]-N,N,2-trimethyl-benzamide | 510.2 | 510 |
| 434 | 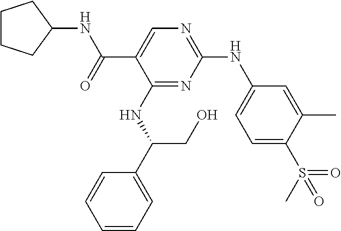 [SMILES: Cc1cc(Nc2ncc(C(=O)NC3CCCC3)c(N[C@H](CO)c3ccccc3)n2)ccc1S(C)(=O)=O] | N-cyclopentyl-4-[[(1S)-2-hydroxy-1- phenyl-ethyl]amino]-2-(3-methyl-4- methylsulfonyl-anilino)pyrimidine-5- carboxamide | 510.2 | 510 |
| 435 | 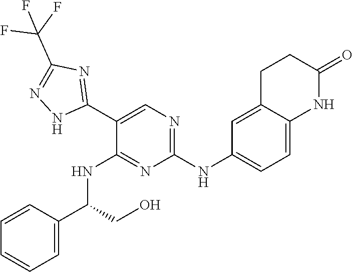 [SMILES: O=C1CCc2cc(Nc3ncc(-c4nc(C(F)(F)F)n[nH]4)c(N[C@H](CO)c4ccccc4)n3)ccc2N1] | 6-[[4-[[(1S)-2-hydroxy-1-phenyl- ethyl]amino]-5-[3-(trifluoromethyl)- 1H-1,2,4-triazol-5-yl]pyrimidin-2- yl]amino]-3,4-dihydro-1H-quinolin-2- one | 511.2 | 511 |
| 436 | 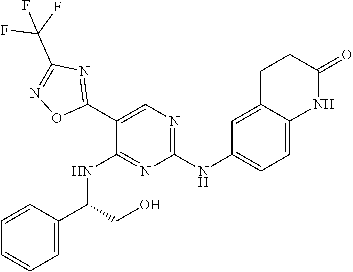 [SMILES: O=C1CCc2cc(Nc3ncc(-c4nc(C(F)(F)F)no4)c(N[C@H](CO)c4ccccc4)n3)ccc2N1] | 6-[[4-[[(1S)-2-hydroxy-1-phenyl- ethyl]amino]-5-[3-(trifluoromethyl)- 1,2,4-oxadiazol-5-yl]pyrimidin-2- yl]amino]-3,4-dihydro-1H-quinolin-2- one | 512.2 | 512 |
| 437 | 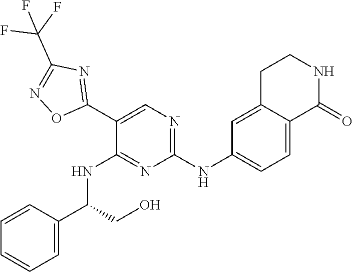 [SMILES: O=C1NCCc2cc(Nc3ncc(-c4nc(C(F)(F)F)no4)c(N[C@H](CO)c4ccccc4)n3)ccc21] | 6-[[4-[[(1S)-2-hydroxy-1-phenyl- ethyl]amino]-5-[3-(trifluoromethyl)- 1,2,4-oxadiazol-5-yl]pyrimidin-2- yl]amino]-3,4-dihydro-2H- isoquinolin-1-one | 512.2 | 512 |
| 438 | 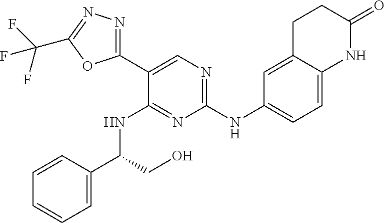 [SMILES: O=C1CCc2cc(Nc3ncc(-c4nnc(C(F)(F)F)o4)c(N[C@H](CO)c4ccccc4)n3)ccc2N1] | 6-[[4-[[(1S)-2-hydroxy-1-phenyl- ethyl]amino]-5-[5-(trifluoromethyl)- 1,3,4-oxadiazol-2-yl]pyrimidin-2- yl]amino]-3,4-dihydro-1H-quinolin-2- one | 512.2 | 512 |
| 439 | 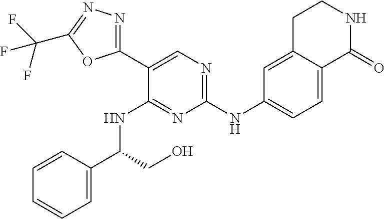 [SMILES: O=C1NCCc2cc(Nc3ncc(-c4nnc(C(F)(F)F)o4)c(N[C@H](CO)c4ccccc4)n3)ccc21] | 6-[[4-[[(1S)-2-hydroxy-1-phenyl- ethyl]amino]-5-[5-(trifluoromethyl)- 1,3,4-oxadiazol-2-yl]pyrimidin-2- yl]amino]-3,4-dihydro-2H- isoquinolin-1-one | 512.2 | 512 |
| 440 | 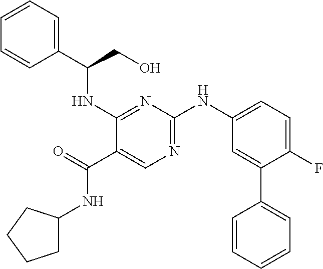 [SMILES: O=C(NC1CCCC1)c1cnc(Nc2ccc(F)c(-c3ccccc3)c2)nc1N[C@H](CO)c1ccccc1] | N-cyclopentyl-2-(4-fluoro-3-phenyl- anilino)-4-[[(1S)-2-hydroxy-1-phenyl- ethyl]amino]pyrimidine-5- carboxamide | 512.2 | 512 |
| 441 | 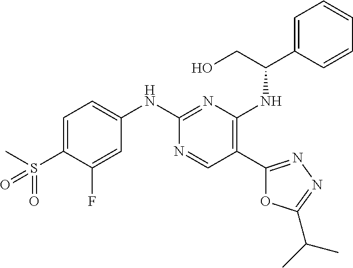 [SMILES: CC(C)c1nnc(-c2cnc(Nc3ccc(S(C)(=O)=O)c(F)c3)nc2N[C@H](CO)c2ccccc2)o1] | (2S)-2-[[2-(3-fluoro-4- methylsulfonyl-anilino)-5-(5- isopropyl-1,3,4-oxadiazol-2- yl)pyrimidin-2-yl]amino]-2-phenyl- ethanol | 513.2 | 513 |
| 442 | 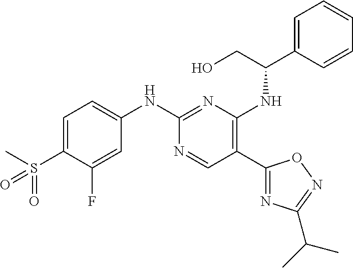 [SMILES: CC(C)c1noc(-c2cnc(Nc3ccc(S(C)(=O)=O)c(F)c3)nc2N[C@H](CO)c2ccccc2)n1] | (2S)-2-[[2-(3-fluoro-4- methylsulfonyl-anilino)-5-(3- isopropyl-1,2,4-oxadiazol-5- yl)pyrimidin-4-yl]amino]-2-phenyl- ethanol | 513.2 | 513 |
| 443 | 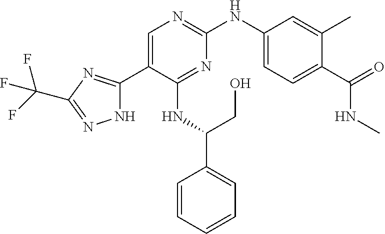 [SMILES: CNC(=O)c1ccc(Nc2ncc(-c3nc(C(F)(F)F)n[nH]3)c(N[C@H](CO)c3ccccc3)n2)cc1C] | 4-[[4-[[(1S)-2-hydroxy-1-phenyl- ethyl]amino]-5-[3-(trifluoromethyl)- 1H-1,2,4-triazol-5-yl]pyrimidin-2- yl]amino]-N,2-dimethyl-benzamide | 513.2 | 513 |
| 444 | 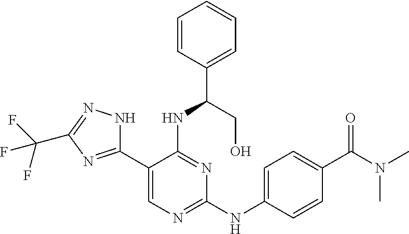 [SMILES: CN(C)C(=O)c1ccc(Nc2ncc(-c3nc(C(F)(F)F)n[nH]3)c(N[C@H](CO)c3ccccc3)n2)cc1] | 4-[[4-[[(1S)-2-hydroxy-1-phenyl- etyl]amino]-5-[3-(trifluoromethyl)- 1H-1,2,4-triazol-5-yl]pyrimidin-2- yl]amino]-N,N-dimethyl-benzamide | 513.2 | 513 |
| 445 | 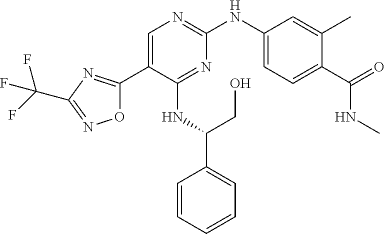 [SMILES: CNC(=O)c1ccc(Nc2ncc(-c3nc(C(F)(F)F)no3)c(N[C@H](CO)c3ccccc3)n2)cc1C] | 4-[[4-[[(1S)-2-hydroxy-1-phenyl- ethyl]amino]-5-[3-(trifluoromethyl)- 1,2,4-oxadiazol-5-yl]pyrimidin-2- yl]amino]-N,2-dimethyl-benzamide | 514.2 | 514 |
| 446 | 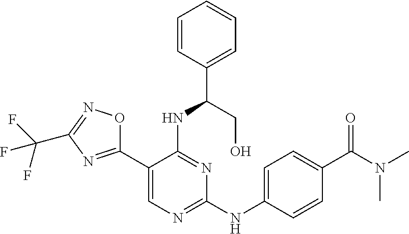 [SMILES: CN(C)C(=O)c1ccc(Nc2ncc(-c3nc(C(F)(F)F)no3)c(N[C@H](CO)c3ccccc3)n2)cc1] | 4-[[4-[[(1S)-2-hydroxy-1-phenyl- ethyl]amino]-5-[3-(trifluoromethyl)- 1,2,4-oxadiazol-5-yl]pyrimidin-2- yl]amino]-N,N-dimethyl-benzamide | 514.2 | 514 |
| 447 | 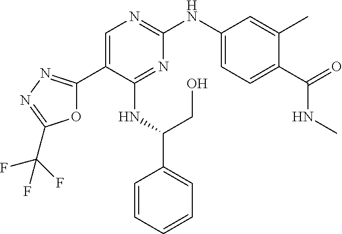 [SMILES: CNC(=O)c1ccc(Nc2ncc(-c3nnc(C(F)(F)F)o3)c(N[C@H](CO)c3ccccc3)n2)cc1C] | 4-[[4-[[(1S)-2-hydroxy-1-phenyl- ethyl]amino]-5-[5-(trifluoromethyl)- 1,3,4-oxadiazol-2-yl]pyrimidin-2- yl]amino]-N,2-dimethyl-benzamide | 514.2 | 514 |
| 448 | 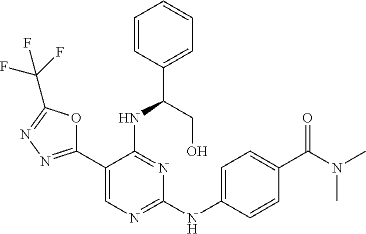 [SMILES: CN(C)C(=O)c1ccc(Nc2ncc(-c3nnc(C(F)(F)F)o3)c(N[C@H](CO)c3ccccc3)n2)cc1] | 4-[[4-[[(1S)-2-hydroxy-1-phenyl- ethyl]amino]-5-[5-(trifluoromethyl)- 1,3,4-oxadiazol-2-yl]pyrimidin-2- yl]amino]-N,N-dimethyl-benzamide | 514.2 | 514 |
| 449 | 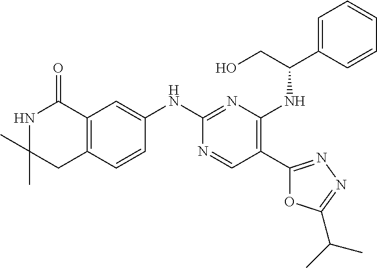 [SMILES: CC(C)c1nnc(-c2cnc(Nc3ccc4c(c3)C(=O)NC(C)(C)C4)nc2N[C@H](CO)c2ccccc2)o1] | 7-[[4-[[(1S)-2-hydroxy-1-phenyl- ethyl]amino]-5-(5-isopopyl-1,3,4- oxadiazol-2-yl)pyrimidin-2- yl]amino]-3,3-dimethyl-2,4- dihydroisoquinolin-1-one | 514.3 | 514 |
| 450 | 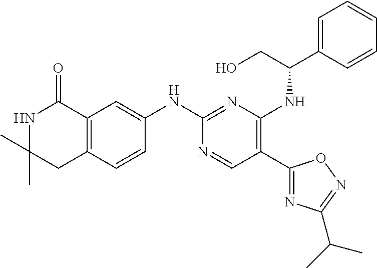 [SMILES: CC(C)c1noc(-c2cnc(Nc3ccc4c(c3)C(=O)NC(C)(C)C4)nc2N[C@H](CO)c2ccccc2)n1] | 7-[[4-[[(1S)-2-hydroxy-1-phenyl- ethyl]amino]-5-(3-isopropyl-1,2,4- oxadiazol-5-yl)pyrimdiin-2- yl]amino]-3,3-dimethyl-2,4- dihydroisoquinolin-1-one | 514.3 | 514 |
| 451 | 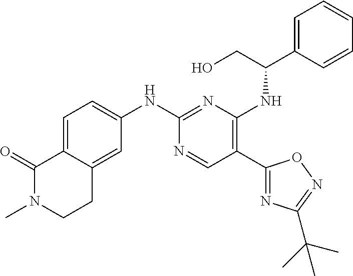 [SMILES: CN1CCc2cc(Nc3ncc(-c4nc(C(C)(C)C)no4)c(N[C@H](CO)c4ccccc4)n3)ccc2C1=O] | 6-[[5-(3-tert-butyl-1,2,4-oxadiazol-5- yl)-4-[[(1S)-2-hydroxy-1-phenyl- ethyl]amino]pyrimidin-2-yl]amino]-2- methyl-3,4-dihydroisoquinolin-1-one | 514.3 | 514 |
| 452 | 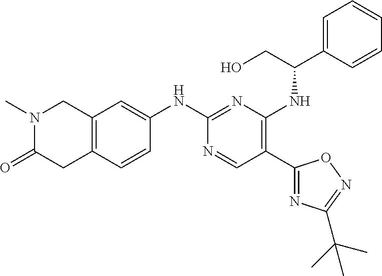 [SMILES: CN1Cc2cc(Nc3ncc(-c4nc(C(C)(C)C)no4)c(N[C@H](CO)c4ccccc4)n3)ccc2CC1=O] | 7-[[5-(3-tert-butyl-1,2,4-oxadiazol-5- yl)-4-[[(1S)-2-hydroxy-1-phenyl- ethyl]amino]pyrimidin-2-yl]amino]-2- methyl-1,4-dihydroisoquinolin-3-one | 514.3 | 514 |
| 453 | 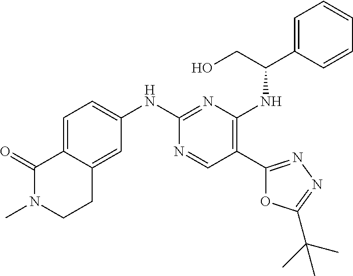 [SMILES: CN1CCc2cc(Nc3ncc(-c4nnc(C(C)(C)C)o4)c(N[C@H](CO)c4ccccc4)n3)ccc2C1=O] | 6-[[5-(5-tert-butyl-1,3,4-oxadiazol-2- yl)-4-[[(1S)-2-hydroxy-1-phenyl- ethyl]amino]pyrimidin-2-yl]amino]-2- methyl-3,4-dihydroisoquinolin-1-one | 514.3 | 514 |
| 454 | 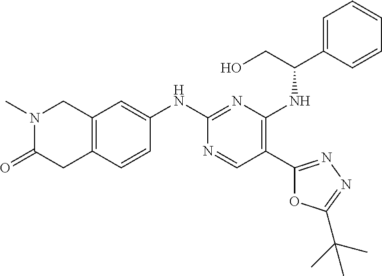 [SMILES: CN1Cc2cc(Nc3ncc(-c4nnc(C(C)(C)C)o4)c(N[C@H](CO)c4ccccc4)n3)ccc2CC1=O] | 7-[[5-(5-tert-butyl-1,3,4-oxadiazol-2- yl)-4-[[(1S)-2-hydroxy-1-phenyl- ethyl]amino]pyrimidin-2-yl]amino]-2- methyl-1,4-dihydroisoquinolin-3-one | 514.3 | 514 |
| 455 | 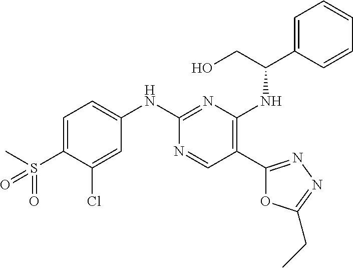 [SMILES: CCc1nnc(-c2cnc(Nc3ccc(S(C)(=O)=O)c(Cl)c3)nc2N[C@H](CO)c2ccccc2)o1] | (2S)-2-[[2-(3-chloro-4- methyslulfonyl-anlino)-5-(5-ethyl- 1,3,4-oxadiazol-2-yl)pyrimidin-4- yl]amino]-2-phenyl-ethanol | 515.1 | 515 |
| 456 | 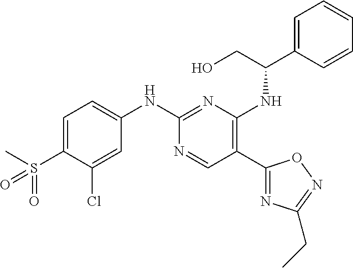 [SMILES: CCc1noc(-c2cnc(Nc3ccc(S(C)(=O)=O)c(Cl)c3)nc2N[C@H](CO)c2ccccc2)n1] | (2S)-2-[[2-(3-chloro-4- methylsulfonyl-anilino)-5-(3-ethyl- 1,2,4-oxadiazol-5-yl)pyrimidin-4- yl]amino]-2-phenyl-ethanol | 515.1 | 515 |
| 457 | 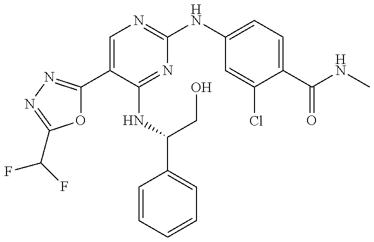 [SMILES: CNC(=O)c1ccc(Nc2ncc(-c3nnc(C(F)F)o3)c(N[C@H](CO)c3ccccc3)n2)cc1Cl] | 2-chloro-4-[[5-[5-(difluoromethyl)- 1,3,4-oxadiazol-2-yl]-4-[[(1S)-2- hydroxy-1-phenyl- ethyl]amino]pyrimidin-2-yl]amino]- N-methyl-benzamide | 516.1 | 516 |
| 458 | 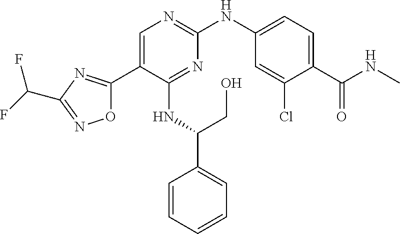 [SMILES: CNC(=O)c1ccc(Nc2ncc(-c3nc(C(F)F)no3)c(N[C@H](CO)c3ccccc3)n2)cc1Cl] | 2-chloro-4-[[5-[3-(difluoromethyl)- 1,2,4-oxadiazol-5-yl]-4-[[(1S)-2- hydroxy-1-phenyl- ethyl]amino]pyrimidin-2-yl]amino]- N-methyl-benzamide | 516.1 | 516 |
| 459 | 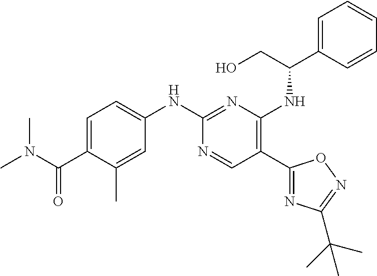 [SMILES: Cc1cc(Nc2ncc(-c3nc(C(C)(C)C)no3)c(N[C@H](CO)c3ccccc3)n2)ccc1C(=O)N(C)C] | 4-[[5-(3-tert-butyl-1,2,4-oxadiazol-5- yl)-4-[[(1S)-2-hydroxy-1-phenyl- ethyl]amino]pyrimidin-2-yl]amino]- N,N,2-trimethyl-benzamide | 516.3 | 516 |
| 460 | 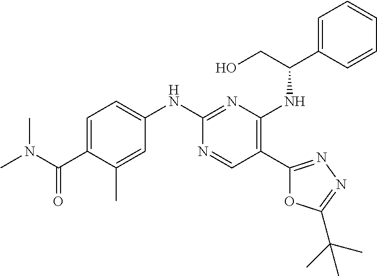 [SMILES: Cc1cc(Nc2ncc(-c3nnc(C(C)(C)C)o3)c(N[C@H](CO)c3ccccc3)n2)ccc1C(=O)N(C)C] | 4-[[5-(5-tert-butyl-1,3,4-oxadiazol-2- yl)-4-[[(2S)-2-hydroxy-1-phenyl- ethyl]amino]pyrimidin-2-yl]amino]- N,N,2-trimethyl-benzamide | 516.3 | 516 |
| 461 | 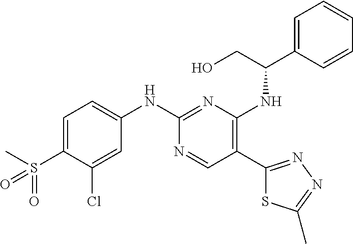 [SMILES: Cc1nnc(-c2cnc(Nc3ccc(S(C)(=O)=O)c(Cl)c3)nc2N[C@H](CO)c2ccccc2)s1] | (2S)-2-[[2-(3-chloro-4- methylsulfonyl-anilino)-5-(5-methyl- 1,3,4-thiadiazol-2-yl)pyrimidin-4- yl]amino]-2-phenyl-ethanol | 517.1 | 517 |
| 462 | 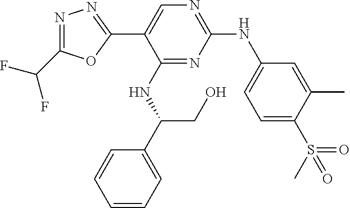 [SMILES: Cc1cc(Nc2ncc(-c3nnc(C(F)F)o3)c(N[C@H](CO)c3ccccc3)n2)ccc1S(C)(=O)=O] | (2S)-2-[[5-[5-(difluoromethyl)-1,3,4- oxadiazol-2-yl]-2-(3-methyl-4- methylsulfonyl-anilino)pyrimidin-4- yl]amino]-2-phenyl-ethanol | 517.1 | 517 |
| 463 | 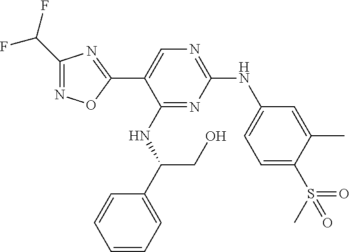 [SMILES: Cc1cc(Nc2ncc(-c3nc(C(F)F)no3)c(N[C@H](CO)c3ccccc3)n2)ccc1S(C)(=O)=O] | (2S)-2-[[5-[3-(difluoromethyl)-1,2,4- oxadiazol-5-yl]-2-(3-methyl-4- methylsulfonyl-anilino)pyrimidin-4- yl]amino]-2-phenyl-ethanol | 517.1 | 517 |
| 464 | 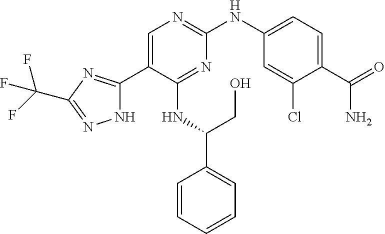 [SMILES: NC(=O)c1ccc(Nc2ncc(-c3nc(C(F)(F)F)n[nH]3)c(N[C@H](CO)c3ccccc3)n2)cc1Cl] | 2-chloro-4-[[4-[[(1S)-2-hydroxy-1- phenyl-ethyl]amino]-5-[3- (trifluoromethyl)-1H-1,2,4-triazol-5- yl]pyrimidin-2-yl]amino]benzamide | 519.1 | 519 |
| 465 | 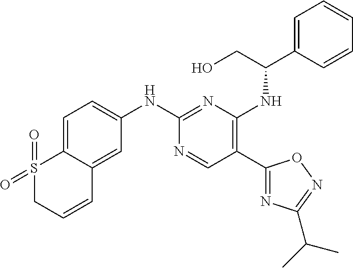 [SMILES: CC(C)c1noc(-c2cnc(Nc3ccc4c(c3)C=CCS4(=O)=O)nc2N[C@H](CO)c2ccccc2)n1] | (2S)-2-[[2-[(1,1-dioxo-2H- thiochromen-6-yl)amino]-5-(3- isopropyl-1,2,4-oxadiazol-5- yl)pyrimidin-4-yl]amino]-2-phenyl- ethanol | 519.2 | 519 |
| 466 | 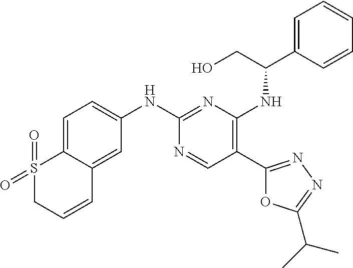 [SMILES: CC(C)c1nnc(-c2cnc(Nc3ccc4c(c3)C=CCS4(=O)=O)nc2N[C@H](CO)c2ccccc2)o1] | (2S)-2-[[2-[(1,1-dioxo-2H- thiochromen-6-yl)amino]-5-(5- isopropyl-1,3,4-oxadiazol-2- yl)pyrimidin-4-yl]amino]-2-phenyl- ethanol | 519.2 | 519 |
| 467 | 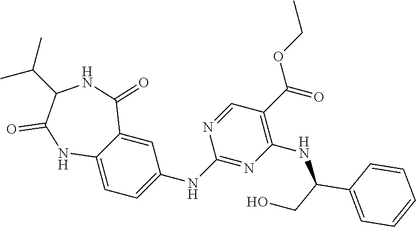 [SMILES: CCOC(=O)c1cnc(Nc2ccc3c(c2)C(=O)NC(C(C)C)C(=O)N3)nc1N[C@H](CO)c1ccccc1] | ethyl 4-[[(1S)-2-hydroxy-1-phenyl- ethyl]amino]-2-[(3-isopropyl-2,5- dioxo-3,4-dihydro-1H-1,4- benzodiazepin-7- yl)amino]pyrimidine-5-carboxylate | 519.2 | 519 |
| 468 | 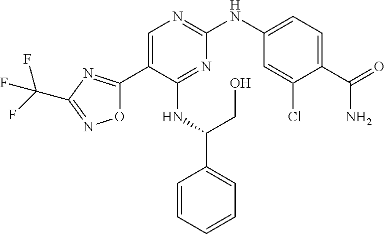 [SMILES: NC(=O)c1ccc(Nc2ncc(-c3nc(C(F)(F)F)no3)c(N[C@H](CO)c3ccccc3)n2)cc1Cl] | 2-chloro-4-[[4-[[(1S)-2-hydroxy-1- phenyl-ethyl]amino]-5-[3- (trifluoromethyl)-1,2,4-oxadiazol-5- yl]pyrimidin-2-yl]amino]benzamide | 520.1 | 520 |
| 469 | 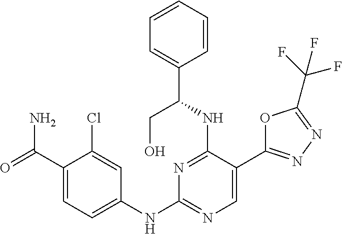 [SMILES: NC(=O)c1ccc(Nc2ncc(-c3nnc(C(F)(F)F)o3)c(N[C@H](CO)c3ccccc3)n2)cc1Cl] | 2-chloro-4-[[4-[[(1S)-2-hydroxy-1- phenyl-ethyl]amino]-5-[5- (trifluoromethyl)-1,3,4-oxadiazol-2- yl]pyrimidin-2-yl]amino]benzamide | 520.1 | 520 |
| 470 | 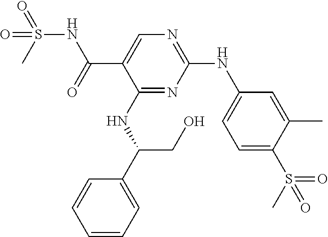 [SMILES: Cc1cc(Nc2ncc(C(=O)NS(C)(=O)=O)c(N[C@H](CO)c3ccccc3)n2)ccc1S(C)(=O)=O] | 4-[[(1S)-2-hydroxy-1-phenyl- ethyl]amino]-2-(3-methyl-4- methylsulfonyl-anilino)-N- methylsulfonyl-pyrimidine-5- carboxamide | 520.1 | 520 |
| 471 | 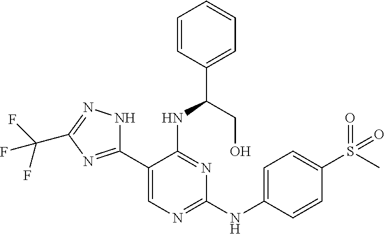 [SMILES: CS(=O)(=O)c1ccc(Nc2ncc(-c3nc(C(F)(F)F)n[nH]3)c(N[C@H](CO)c3ccccc3)n2)cc1] | (2S)-2-[[2-(4-methylsulfonylanilino)- 5-[3-(trifluoromethyl)-1H-1,2,4- triazol-5-yl]pyrimidin-4-yl]amino]-2- phenyl-ethanol | 520.1 | 520 |
| 472 | 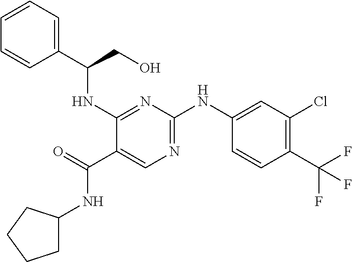 [SMILES: O=C(NC1CCCC1)c1cnc(Nc2ccc(C(F)(F)F)c(Cl)c2)nc1N[C@H](CO)c1ccccc1] | 2-[3-chloro-4- (trifluoromethyl)anilino]-N- cyclopentyl-4-[[(1S)-2-hydroxy-1- phenyl-ethyl]amino]-pyrimidine-5- carboxamide | 520.2 | 520 |
| 473 | 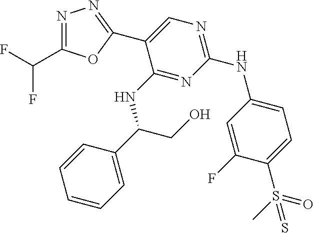 [SMILES: CS(=O)(=S)c1ccc(Nc2ncc(-c3nnc(C(F)F)o3)c(N[C@H](CO)c3ccccc3)n2)cc1F] | (2S)-2-[[5-[5-(difluoromethyl)-1,3,4- oxadiazol-2-yl]-2-(3-fluoro-4- methylsulfonyl-anilino)pyrimidin-4- yl]amino]-2-phenyl-ethanol | 521.1 | 521 |
| 474 | 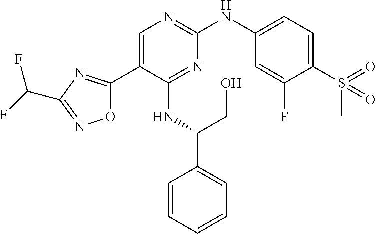 [SMILES: CS(=O)(=O)c1ccc(Nc2ncc(-c3nc(C(F)F)no3)c(N[C@H](CO)c3ccccc3)n2)cc1F] | (2S)-2-[[5-[3-(difluoromethyl)-1,2,4- oxadiazol-5-yl]-2-(3-fluoro-4- methylsulfonyl-anilino)pyrimidin-4- yl]amino]-2-phenyl-ethanol | 521.1 | 521 |
| 475 | 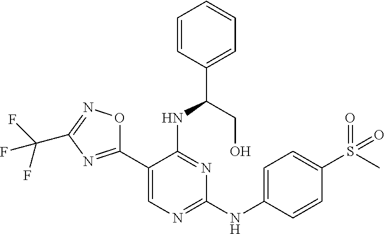 [SMILES: CS(=O)(=O)c1ccc(Nc2ncc(-c3nc(C(F)(F)F)no3)c(N[C@H](CO)c3ccccc3)n2)cc1] | (2S)-2-[[2-(4-methylsulfonylanilino)- 5-[3-(trifluoromethyl)-1,2,4- oxadiazol-5-yl]pyrimidin-4- yl]amino]-2-phenyl-ethanol | 521.1 | 521 |
| 476 | 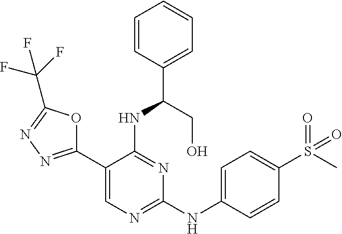 [SMILES: CS(=O)(=O)c1ccc(Nc2ncc(-c3nnc(C(F)(F)F)o3)c(N[C@H](CO)c3ccccc3)n2)cc1] | (2S)-2-[[2-(4-methylsulfonylanilino)- 5-[5-(trifluoromethyl)-1,3,4- oxadiazol-2-yl]pyrimidin-4- yl]amino]-2-phenyl-ethanol | 521.1 | 521 |
| 477 | 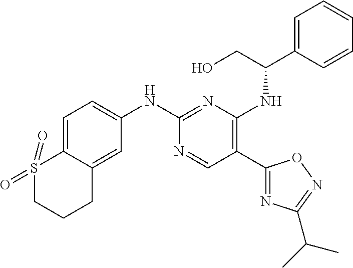 [SMILES: CC(C)c1noc(-c2cnc(Nc3ccc4c(c3)CCCS4(=O)=O)nc2N[C@H](CO)c2ccccc2)n1] | (2S)-2-[[2-[(1,1-dioxo-3,4-dihydro- 2H-thiochromen-6-yl)amino]-5-93- isopropyl-1,2,4-oxadiazol-5- yl)pyrimidin-4-yl]amino]-2-phenyl- ethanol | 521.2 | 521 |
| 478 | 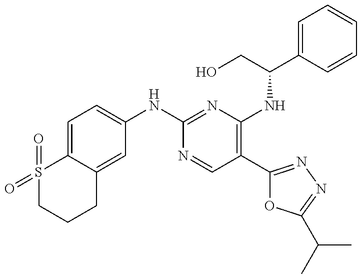 [SMILES: CC(C)c1nnc(-c2cnc(Nc3ccc4c(c3)CCCS4(=O)=O)nc2N[C@H](CO)c2ccccc2)o1] | (2S)-2-[[2-[(1,1-dioxo-3,4-dihydro- 2H-thiochromen-6-yl)amino]-5-(5- isopropyl-1,3,4-oxadiazol-2- yl)pyrimidin-4-yl]amino]-2-phenyl- ethanol | 521.2 | 521 |
| 479 | 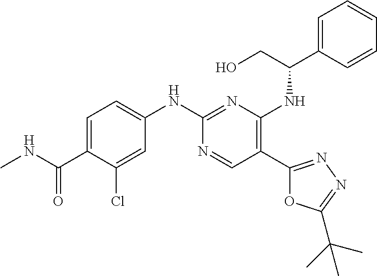 [SMILES: CNC(=O)c1ccc(Nc2ncc(-c3nnc(C(C)(C)C)o3)c(N[C@H](CO)c3ccccc3)n2)cc1Cl] | 4-[[5-(5-tert-butyl-1,3,4-oxadiazol-2- yl)-4-[[(1S)-2-hydroxy-1-phenyl- ethyl]amino]pyrimidin-2-yl]amino]-2- chloro-N-methyl-benzamide | 522.2 | 522 |
| 480 | 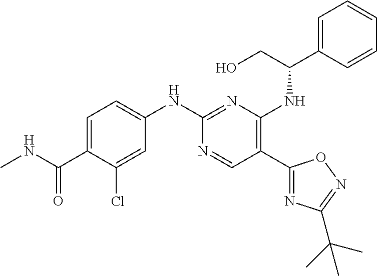 [SMILES: CNC(=O)c1ccc(Nc2ncc(-c3nc(C(C)(C)C)no3)c(N[C@H](CO)c3ccccc3)n2)cc1Cl] | 4-[[5-(3-tert-butyl-1,2,4-oxadiazol-5- yl)-4-[[(1S)-2-hydroxy-1-phenyl- ethyl]amino]pyrimidin-2-yl]amino]-2- chloro-N-methyl-benzamide | 522.2 | 522 |
| 481 | 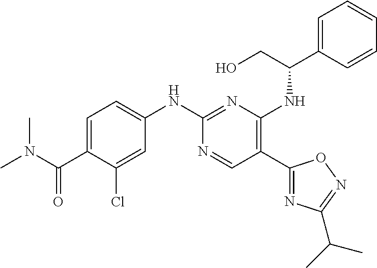 [SMILES: CC(C)c1noc(-c2cnc(Nc3ccc(C(=O)N(C)C)c(Cl)c3)nc2N[C@H](CO)c2ccccc2)n1] | 2-chloro-4-[[4-[[(1S)-2-hydroxy-1- phenyl-ethyl]amino]-5-(3-isopropyl- 1,2,4-oxadiazol-5-yl)pyrimidin-2- yl]amino]-N,N-dimethyl-benzamide | 522.2 | 522 |
| 482 | 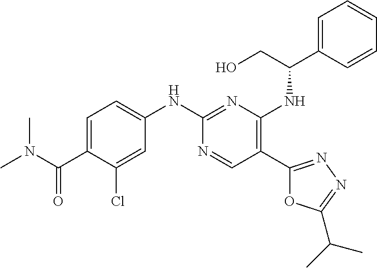 [SMILES: CC(C)c1nnc(-c2cnc(Nc3ccc(C(=O)N(C)C)c(Cl)c3)nc2N[C@H](CO)c2ccccc2)o1] | 2-chloro-4-[[4-[[(1S)-2-hydroxy-1- phenyl-ethyl]amino]-5-(5-isopropyl- 1,3,4-oxadiazol-2-yl)pyrimidin-2- yl]amino]-N,N-dimethyl-benzamide | 522.2 | 522 |
| 483 | 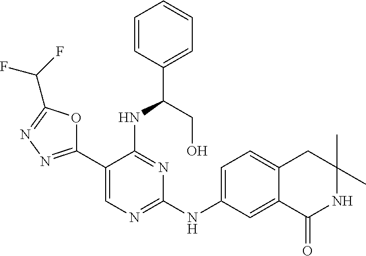 [SMILES: CC1(C)Cc2ccc(Nc3ncc(-c4nnc(C(F)F)o4)c(N[C@H](CO)c4ccccc4)n3)cc2C(=O)N1] | 7-[[5-[5-(difluoromethyl)-1,3,4- oxadiazol-2-yl]-4-[[(1S)-2-hydroxy-1- phenyl-ethyl]amino]pyrimidin-2- yl]amino]-3,3-dimethyl-2,4- dihydroisoquinolin-1-one | 522.2 | 522 |
| 484 | 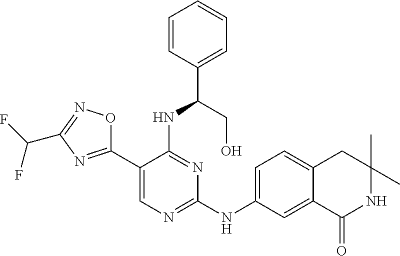 [SMILES: CC1(C)Cc2ccc(Nc3ncc(-c4nc(C(F)F)no4)c(N[C@H](CO)c4ccccc4)n3)cc2C(=O)N1] | 7-[[5-[3-(difluoromethyl)-1,2,4- oxadiazol-5-yl]-4-[[(1S)-2-hydroxy-1- phenyl-ethyl]amino]pyrimidin-2- yl]amino]-3,3-dimethyl-2,4- dihdyroisoquinolin-1-one | 522.2 | 522 |
| 485 | 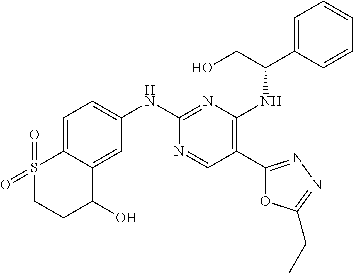 [SMILES: CCc1nnc(-c2cnc(Nc3ccc4c(c3)C(O)CCS4(=O)=O)nc2N[C@H](CO)c2ccccc2)o1] | 6-[[5-(5-ethyl-1,3,4-oxadiazol-2-yl)- 4-[[(1S)-2-hydroxy-1-phenyl- ethyl]amino]pyrimidin-2-yl]amino]- 1,1-dioxo-3,4-dihydro-2H- thiochromen-4-ol | 523.2 | 523 |
| 486 | 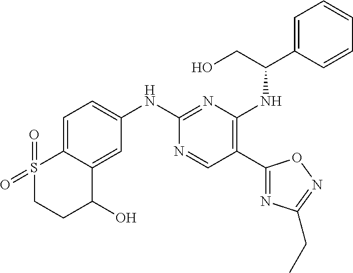 [SMILES: CCc1noc(-c2cnc(Nc3ccc4c(c3)C(O)CCS4(=O)=O)nc2N[C@H](CO)c2ccccc2)n1] | 6-[[5-(3-ethyl-1,2,4-oxadiazol-5-yl)- 4-[[(1S)-2-hydroxy-1-phenyl- ethyl]amino]pyrimidin-2-yl]amino]- 1,1-dioxo-3,4-dihydro-2H- thiochromen-4-ol | 523.2 | 523 |
| 487 | 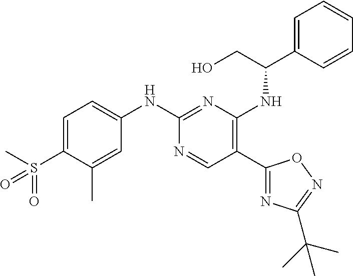 [SMILES: Cc1cc(Nc2ncc(-c3nc(C(C)(C)C)no3)c(N[C@H](CO)c3ccccc3)n2)ccc1S(C)(=O)=O] | (2S)-2-[[5-(5-tert-butyl-1,3,4- oxadiazol-2-yl)-2-(3-methyl-4- methylsulfonyl-anilino)pyrimidin-4- yl]amino]-2-phenyl-ethanol | 523.2 | 523 |
| 488 | 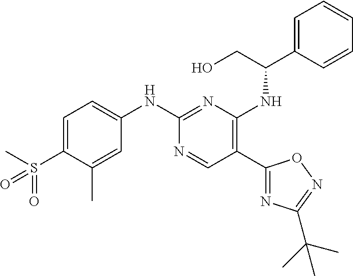 [SMILES: Cc1cc(Nc2ncc(-c3nc(C(C)(C)C)no3)c(N[C@H](CO)c3ccccc3)n2)ccc1S(C)(=O)=O] | (2S)-2-[[5-(3-tert-butyl-1,2,4- oxadiazol-5-yl)-2-(3-methyl-4- methylsulfonyl-anilino)pyrimidin-4- yl]amino]-2-phenyl-ethanol | 523.2 | 523 |
| 489 | 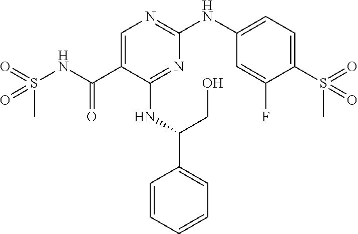 [SMILES: CS(=O)(=O)NC(=O)c1cnc(Nc2ccc(S(C)(=O)=O)c(F)c2)nc1N[C@H](CO)c1ccccc1] | 2-(3-fluoro-4-methylsulfonyl- anilino)-4-[[(1S)-2-hydroxy-1-phenyl- ethyl]amino]-N-methylsulfonyl- pyrimidine-5-carboxamide | 524.1 | 524 |
| 490 | 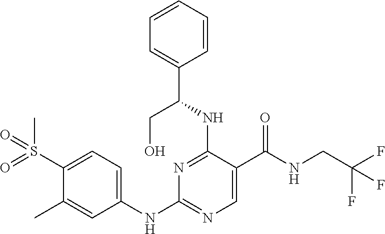 [SMILES: Cc1cc(Nc2ncc(C(=O)NCC(F)(F)F)c(N[C@H](CO)c3ccccc3)n2)ccc1S(C)(=O)=O] | 4-[[(1S)-2-hydroxy-1-phenyl- ethyl]amino]-2-(3-methyl-4- methylsulfonyl-anilino)-N-(2,2,2- trifluoroethyl)-pyrimidine-5- carboxamide | 524.2 | 524 |
| 491 | 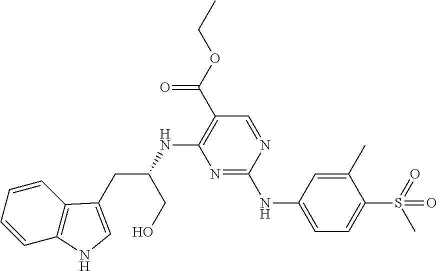 [SMILES: CCOC(=O)c1cnc(Nc2ccc(S(C)(=O)=O)c(C)c2)nc1N[C@H](CO)Cc1c[nH]c2ccccc12] | ethyl 4-[[(1S)-2-(hydroxymethyl)-2- (1H-indol-3-yl)ethyl]amino]-2-(3- methyl-4-methylsulfonyl- anilino)pyrimnidine-5-carboxylate | 524.2 | 524 |
| 492 | 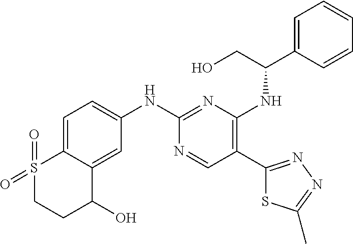 [SMILES: Cc1nnc(-c2cnc(Nc3ccc4c(c3)C(O)CCS4(=O)=O)nc2N[C@H](CO)c2ccccc2)s1] | 6-[[4-[[(1S)-2-hydroxy-1-phenyl- ethyl]amino]-5-(5-methyl-1,3,4- thiadiazol-2-yl)pyrimidin-2- yl]amino]-1,1-dioxo-3,4-dihydro-2H- thiochromen-4-ol | 525.1 | 525 |
| 493 | 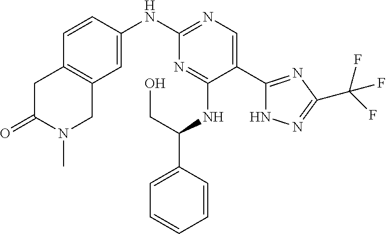 [SMILES: CN1Cc2cc(Nc3ncc(-c4nc(C(F)(F)F)n[nH]4)c(N[C@H](CO)c4ccccc4)n3)ccc2CC1=O] | 7-[[4-[[(1S)-2-hydroxy-1-phenyl- ethyl]amino]-5-[3-(trifluoromethyl)- 1H-1,2,4-triazol-5-yl]pyrimidin-2- yl]amino]-2-methyl-1,4-dihydro- isoquinolin-3-one | 525.2 | 525 |
| 494 | 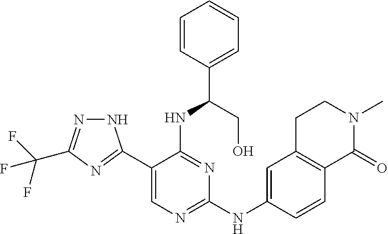 [SMILES: CN1CCc2cc(Nc3ncc(-c4nc(C(F)(F)F)n[nH]4)c(N[C@H](CO)c4ccccc4)n3)ccc2C1=O] | 6-[[4-[[(1S)-2-hydroxy-1-phenyl- ethyl]amino]-5-[3-(trifluoromethyl)- 1H-1,2,4-triazol-5-yl]pyrimidin-2- yl]amino]-2-methyl-3,4-dihydro- isoquinolin-1-one | 525.2 | 525 |
| 495 | 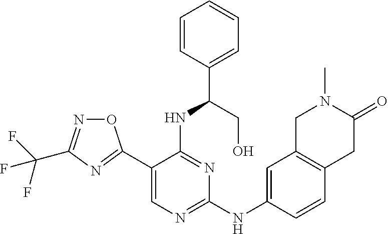 [SMILES: CN1Cc2cc(Nc3ncc(-c4nc(C(F)(F)F)no4)c(N[C@H](CO)c4ccccc4)n3)ccc2CC1=O] | 7-[[4-[[(1S)-2-hydroxy-1-phenyl- ethyl]amino]-5-[3-(trifluoromethyl)- 1,2,4-oxadiazol-5-yl]pyrimidin-2- yl]amino]-2-methyl-1,4-dihydro- isoquinolin-3-one | 526.2 | 526 |
| 496 | 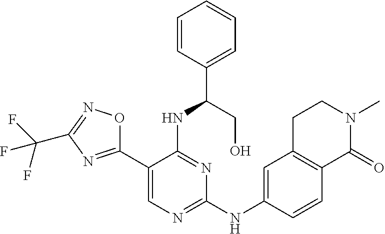 [SMILES: CN1CCc2cc(Nc3ncc(-c4nc(C(F)(F)F)no4)c(N[C@H](CO)c4ccccc4)n3)ccc2C1=O] | 6-[[4-[[(1S)-2-hydroxy-1-phenyl- ethyl]amino]-5-[3-(trifluoromethyl)- 1,2,4-oxadiazol-5-yl]pyrimidin-2- yl]amino]-2-methyl-3,4-dihydro- isoquinolin-1-one | 526.2 | 526 |
| 497 | 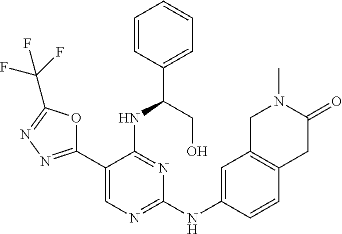 [SMILES: CN1Cc2cc(Nc3ncc(-c4nnc(C(F)(F)F)o4)c(N[C@H](CO)c4ccccc4)n3)ccc2CC1=O] | 7-[[4-[[(1S)-2-hydroxy-1-phenyl- ethyl]amino]-5-[5-(trifluoromethyl)- 1,3,4-oxadiazol-2-yl]pyrimidin-2- yl]amino]-2-methyl-1,4- dihydroisoquinolin-3-one | 526.2 | 526 |
| 498 | 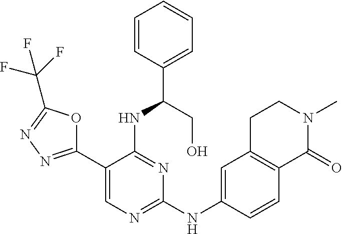 [SMILES: CN1CCc2cc(Nc3ncc(-c4nnc(C(F)(F)F)o4)c(N[C@H](CO)c4ccccc4)n3)ccc2C1=O] | 6-[[4-[[(1S)-2-hydroxy-1-phenyl- ethyl]amino]-5-[5-(trifluoromethyl)- 1,3,4-oxadiazol-2-yl]pyrimidin-2- yl]amino]-2-methyl-3,4- dihydroisoquinolin-1-one | 526.2 | 526 |
| 499 | 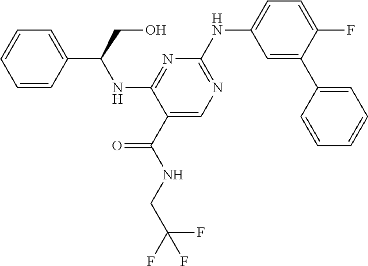 [SMILES: O=C(NCC(F)(F)F)c1cnc(Nc2ccc(F)c(-c3ccccc3)c2)nc1N[C@H](CO)c1ccccc1] | 2-(4-fluoro-3-phenyl-anilino)-4- [[(1S)-2-hydroxy-1-phenyl- ethyl]amino]-N-(2,2,2-trifluoroethyl)- pyrimidine-5-carboxamide | 526.2 | 526 |
| 500 | 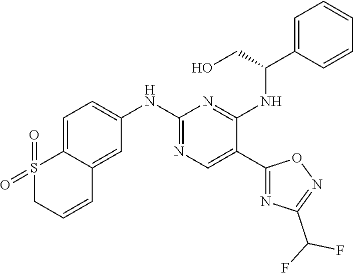 [SMILES: O=S1(=O)CC=Cc2cc(Nc3ncc(-c4nc(C(F)F)no4)c(N[C@H](CO)c4ccccc4)n3)ccc21] | (2S)-2-[[5-[3-(difluoromethyl)-1,2,4- oxadiazol-5-yl]-2-[(1,1-dioxo-2H- thiochromen-6-yl)amino)pyrimidin-4- yl]amino]-2-phenyl-ethanol | 527.1 | 527 |
| 501 | 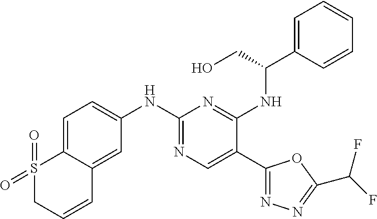 [SMILES: O=S1(=O)CC=Cc2cc(Nc3ncc(-c4nnc(C(F)F)o4)c(N[C@H](CO)c4ccccc4)n3)ccc21] | (2S)-2-[[5-[5-(difluoromethyl)-1,3,4- oxadiazol-2-yl]-2-[(1,1-dioxo-2H- thiochromen-6-yl)amino]pyrimidin-4- yl]amio]-2-phenyl-ethanol | 527.1 | 527 |
| 502 | 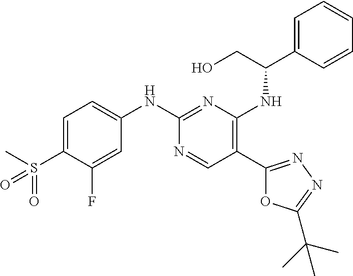 [SMILES: CC(C)(C)c1nnc(-c2cnc(Nc3ccc(S(C)(=O)=O)c(F)c3)nc2N[C@H](CO)c2ccccc2)o1] | (2S)-2-[[5-(5-tert-butyl-1,3,4- oxadiazol-2-yl)-2-(3-fluoro-4- methylsulfonyl-anilino)pyrimidin-4- yl]amino]-2-phenyl-ethanol | 527.2 | 527 |
| 503 | 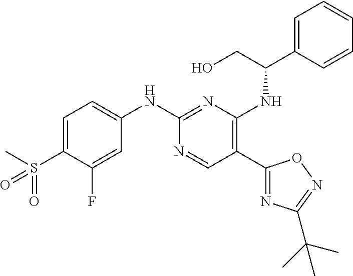 [SMILES: CC(C)(C)c1noc(-c2cnc(Nc3ccc(S(C)(=O)=O)c(F)c3)nc2N[C@H](CO)c2ccccc2)n1] | (2S)-2-[[5-(3-tert-butyl-1,2,4- oxadiazol-5-yl)-2-(3-fluoro-4- methylsulfonyl-anilino)pyrimidin-4- yl]amino]-2-phenyl-ethanol | 527.2 | 527 |
| 504 | 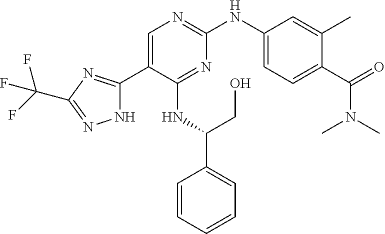 [SMILES: Cc1cc(Nc2ncc(-c3nc(C(F)(F)F)n[nH]3)c(N[C@H](CO)c3ccccc3)n2)ccc1C(=O)N(C)C] | 4-[[4-[[(1S)-2-hydroxy-1-phenyl- ethyl]amino]-5-[3-(trifluoromethyl)- 1H-1,2,4-triazol-5-yl]pyrimidin-2- yl]amino]-N,N,2-trimethyl-benzamide | 527.2 | 527 |
| 505 | 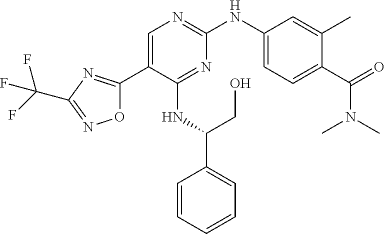 [SMILES: Cc1cc(Nc2ncc(-c3nc(C(F)(F)F)no3)c(N[C@H](CO)c3ccccc3)n2)ccc1C(=O)N(C)C] | 4-[[4-[[(1S)-2-hydroxy-1-phenyl- ethyl]amino]-5-[3-(trifluoromethyl)- 1,2,4-oxadiazol-5-yl]pyrimidin-2- yl]amino]-N,N,2-timethyl-benzamide | 528.2 | 528 |
| 506 | 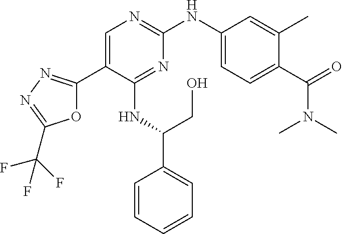 [SMILES: Cc1cc(Nc2ncc(-c3nnc(C(F)(F)F)o3)c(N[C@H](CO)c3ccccc3)n2)ccc1C(=O)N(C)C] | 4-[[4-[[(1S)-2-hydroxy-1-phenyl- ethyl]amino]-5-[5-(trifluoromethyl)- 1,3,4-oxadiazol-2-yl]pyrimidin-2- yl]amino]-N,N,2-trimethyl-benzamide | 528.2 | 528 |
| 507 | 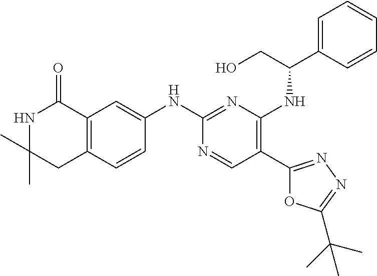 [SMILES: CC1(C)Cc2ccc(Nc3ncc(-c4nnc(C(C)(C)C)o4)c(N[C@H](CO)c4ccccc4)n3)cc2C(=O)N1] | 7-[[5-(5-tert-butyl-1,3,4-oxadiazol-2- yl)-4-[[(1S)-2-hydroxy-1-phenyl- ethyl]amino]pyrimidin-2-yl]amino]- 3,3-dimethyl-2,4-dihydroisoquinolin- 1-one | 528.3 | 528 |
| 508 | 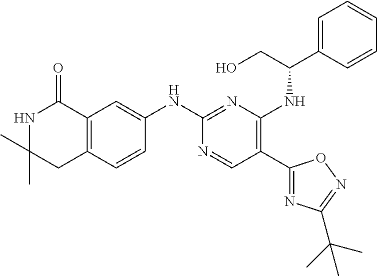 [SMILES: CC1(C)Cc2ccc(Nc3ncc(-c4nc(C(C)(C)C)no4)c(N[C@H](CO)c4ccccc4)n3)cc2C(=O)N1] | 7-[[5-(3-tert-butyl-1,2,4-oxadiazol-5- yl)-4-[[(1S)-2-hydroxy-1-phenyl- ethyl]amino]pyrimidin-2-yl]amino]- 3,3-dimethyl-2,4-dihydroisoquinolin- 1-one | 528.3 | 528 |
| 509 | 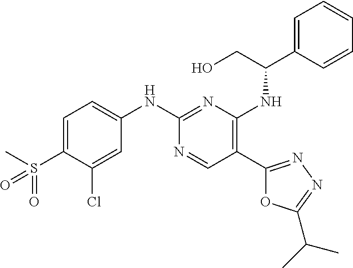 [SMILES: CC(C)c1nnc(-c2cnc(Nc3ccc(S(C)(=O)=O)c(Cl)c3)nc2N[C@H](CO)c2ccccc2)o1] | (2S)-2-[[2-(3-chloro-4- methylsulfonyl-anilino)-5-(5- isopropyl-1,3,4-oxadiazol-2- yl)pyrimidin-4-yl]amino]-2-phenyl- ethanol | 529.1 | 529 |
| 510 | 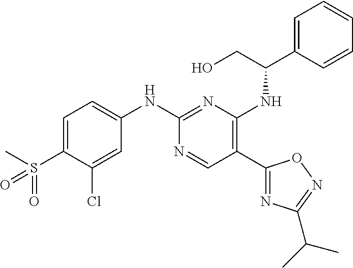 [SMILES: CC(C)c1noc(-c2cnc(Nc3ccc(S(C)(=O)=O)c(Cl)c3)nc2N[C@H](CO)c2ccccc2)n1] | (2S)-2-[[2-(3-chloro-4- methylsulfonyl-anilino)-5-(3- isopropyl-1,2,4-oxadiazol-5- yl)pyrimidin-4-yl]amino]-2-phenyl- ethanol | 529.1 | 529 |
| 511 | 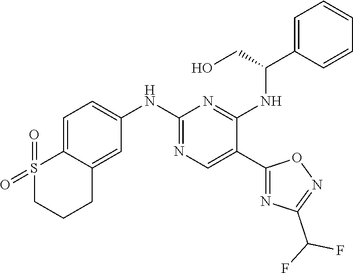 [SMILES: O=S1(=O)CCCc2cc(Nc3ncc(-c4nc(C(F)F)no4)c(N[C@H](CO)c4ccccc4)n3)ccc21] | (2S)-2-[[5-[3-(difluoromethyl)-1,2,4- oxadiazol-5-yl]-2-[(1,1-dioxo-3,4- dihydro-2H-thiochromen-6- yl)amino]pyrimidin-4-yl]amino]-2- phenyl-ethanol | 529.1 | 529 |
| 512 | 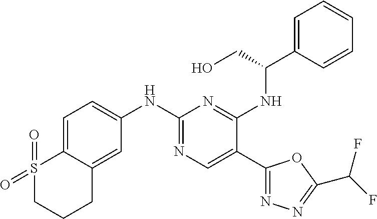 [SMILES: O=S1(=O)CCCc2cc(Nc3ncc(-c4nnc(C(F)F)o4)c(N[C@H](CO)c4ccccc4)n3)ccc21] | (2S)-2-[[5-[5-(difluoromethyl)-1,3,4- oxadiazol-2-yl]-2-[(1,1-dioxo-3,4- dihydro-2H-thiochromen-6- yl)amino]pyrimidni-4-yl]amino]-2- phenyl-ethanol | 529.1 | 529 |
| 513 | 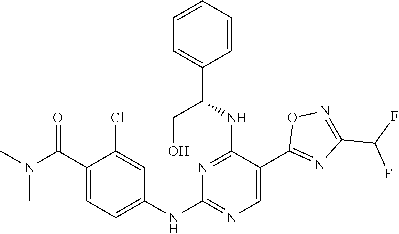 [SMILES: CN(C)C(=O)c1ccc(Nc2ncc(-c3nc(C(F)F)no3)c(N[C@H](CO)c3ccccc3)n2)cc1Cl] | 2-chloro-4-[[5-[3-(difluoromethyl) 1,2,4-oxadiazol-5-yl]-4-[[(1S)-2- hydroxy-1-phenyl- ethyl]amino]pyrimidin-2-yl]amino]- N,N-dimethyl-benzamide | 530.1 | 530 |
| 514 | 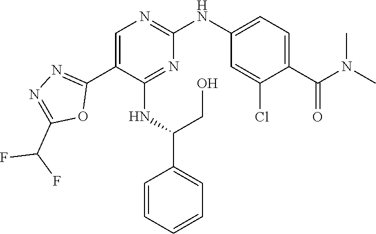 [SMILES: CN(C)C(=O)c1ccc(Nc2ncc(-c3nnc(C(F)F)o3)c(N[C@H](CO)c3ccccc3)n2)cc1Cl] | 2-chloro-4-[[5-[5-(difluoromethyl)- 1,3,4-oxadiazol-2-yl]-4-[[(1S)-2- hydroxy-1-phenyl- ethyl]amino]pyrimidin-2-yl]amino]- N,N-dimethyl-benzamide | 530.2 | 530 |
| 515 | 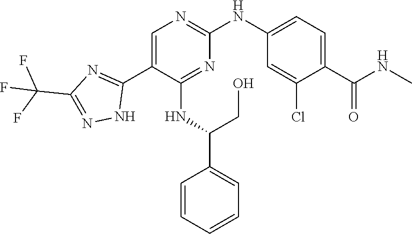 [SMILES: CNC(=O)c1ccc(Nc2ncc(-c3nc(C(F)(F)F)n[nH]3)c(N[C@H](CO)c3ccccc3)n2)cc1Cl] | 2-chloro-4-[[4-[[(1S)-2-hydroxy-1- phenyl-ethyl]amino]-5-[3- (trifluoromethyl)-1H-1,2,4-triazol-5- yl]apyrimidin-2-yl]amino]-N-emethyl- benzamide | 533.1 | 533 |
| 516 | 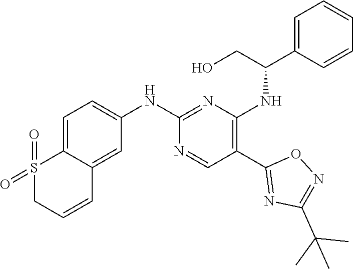 [SMILES: CC(C)(C)c1noc(-c2cnc(Nc3ccc4c(c3)C=CCS4(=O)=O)nc2N[C@H](CO)c2ccccc2)n1] | (2S)-2-[[5-(3-tert-butyl-1,2,4- oxadiazol-5-yl)-2-[(1,1-dioxo-2H- thiochromen-6-yl)amino]pyrimidin-4- yl]amino]-2-phenyl-ethanol | 533.2 | 533 |
| 517 | 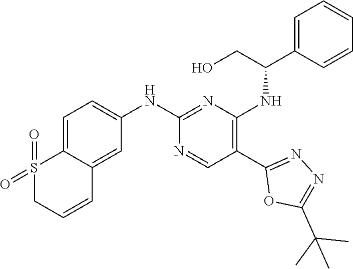 [SMILES: CC(C)(C)c1nnc(-c2cnc(Nc3ccc4c(c3)C=CCS4(=O)=O)nc2N[C@H](CO)c2ccccc2)o1] | (2S)-2-[[5-(5-tert-butyl-1,3,4- oxadiazol-2-yl)-2-[(1,1-dioxo-2H- thiochromen-6-yl)amino]pyrimidin-4- yl]amino]-2-phenyl-ethanol | 533.2 | 533 |
| 518 | 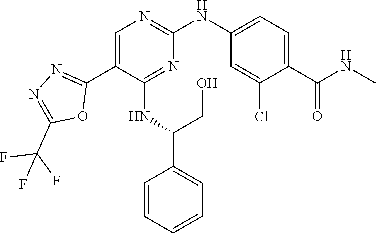 [SMILES: CNC(=O)c1ccc(Nc2ncc(-c3nnc(C(F)(F)F)o3)c(N[C@H](CO)c3ccccc3)n2)cc1Cl] | 2-chloro-4-[[4-[[(1S)-2-hydroxy-1- phenyl-ethhyl]amino]-5-[5- (trifluoromethyl)-1,3,4-oxadiazol-2- yl]pyrimidin-2-yl]amino]-N-methyl- benzamide | 534.1 | 534 |
| 519 | 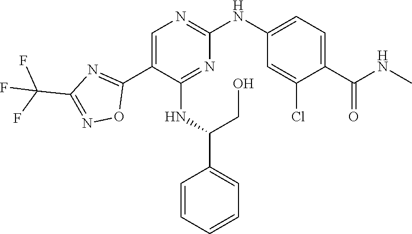 [SMILES: CNC(=O)c1ccc(Nc2ncc(-c3nc(C(F)(F)F)no3)c(N[C@H](CO)c3ccccc3)n2)cc1Cl] | 2-chloro-4-[[4-[[(1S)-2-hydroxy-1- phenyl-ethyl]amino]-5-[3- (trifluoromethyl)-1,2,4-oxadiazol-5- yl]pyrimidin-2-yl]amino]-N-methyl- benzamide | 534.1 | 534 |
| 520 | 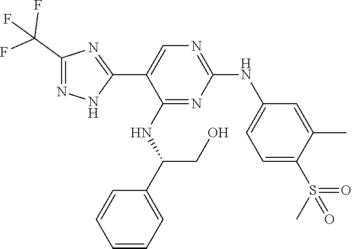 [SMILES: Cc1cc(Nc2ncc(-c3nc(C(F)(F)F)n[nH]3)c(N[C@H](CO)c3ccccc3)n2)ccc1S(C)(=O)=O] | (2S)-2-[[2-(3-methyl-4- methylsulfonyl-anilino)-5-[3- (trifluoromethyl)-1H-1,2,4-triazol-5- y]pyrimidin-4-yl]amino]-2-phenyl- ethanol | 534.2 | 534 |
| 521 | 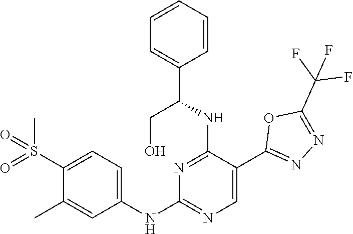 [SMILES: Cc1cc(Nc2ncc(-c3nnc(C(F)(F)F)o3)c(N[C@H](CO)c3ccccc3)n2)ccc1S(C)(=O)=O] | (2S)-2-[[2-(3-methyl-4- methylsulfonyl-anilino)-5-[5- (trifluoromethyl)-1,3,4-oxadiazol-2- yl]pyrimidin-2-yl]amino]-2-phenyl- ethanol | 535.1 | 535 |
| 522 | 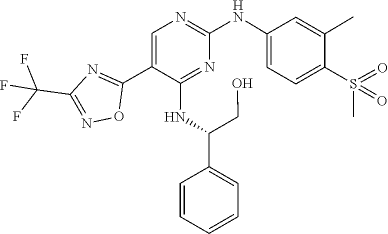 [SMILES: Cc1cc(Nc2ncc(-c3nc(C(F)(F)F)no3)c(N[C@H](CO)c3ccccc3)n2)ccc1S(C)(=O)=O] | (2S)-2-[[2-(3-methyl-4- methylsulfonyl-anilino)-5-[3- (trifluoromethyl)-1,2,4-oxadiazol-5- yl]pyrimidin-4-yl]amino]-2-phenyl- ethanol | 535.1 | 535 |
| 523 | 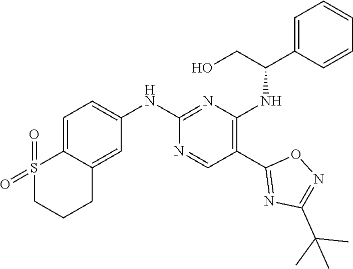 [SMILES: CC(C)(C)c1noc(-c2cnc(Nc3ccc4c(c3)CCCS4(=O)=O)nc2N[C@H](CO)c2ccccc2)n1] | (2S)-2-[[5-(3-tert-butyl-1,2,4- oxadiazol-5-yl)-2-[(1,1-dioxo-3,4- dihydro-2H-thiochromen-6- yl)amino]pyrimidin-4-yl]amino]-2- phenyl-ethanol | 535.2 | 535 |
| 524 | 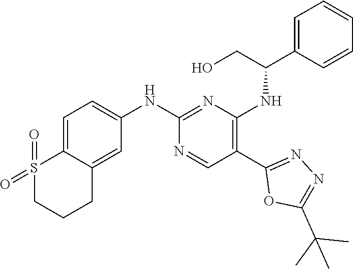 [SMILES: CC(C)(C)c1nnc(-c2cnc(Nc3ccc4c(c3)CCCS4(=O)=O)nc2N[C@H](CO)c2ccccc2)o1] | (2)S-2-[[5-(5-tert-butyl-1,3,4- oxadiazol-2-yl)-2-[(1,1-dioxo-3,4- dihydro-2H-thiochromen-6- yl)amio]pyrimidin-4-yl]amino]-2- phenyl-ethanol | 535.2 | 535 |
| 525 | 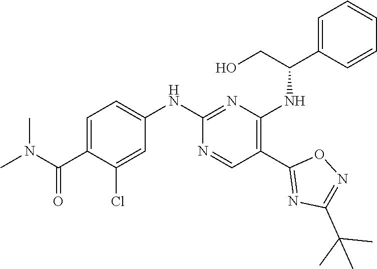 [SMILES: CN(C)C(=O)c1ccc(Nc2ncc(-c3nc(C(C)(C)C)no3)c(N[C@H](CO)c3ccccc3)n2)cc1Cl] | 4-[[5-(3-tert-butyl-1,2,4-oxadiazol-5- yl)-4-[[(1S)-2-hydroxy-1-phenyl- ethyl]amino]pyrimidin-2-yl]amino]-2- chloro-N,N-dimethyl-benzamide | 536.2 | 536 |
| 526 | 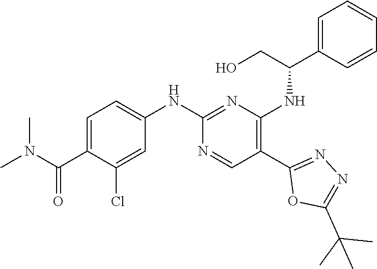 [SMILES: CN(C)C(=O)c1ccc(Nc2ncc(-c3nnc(C(C)(C)C)o3)c(N[C@H](CO)c3ccccc3)n2)cc1Cl] | 4-[[5-(5-tert-butyl-1,3,4-oxadiazol-2- yl)-4-[[(1S)-2-hydroxy-1-phenyl- ethyl]amino]pyrimidin-2-yl]amino]-2- chloro-N,N-dimethyl-benzamide | 536.2 | 536 |
| 527 | 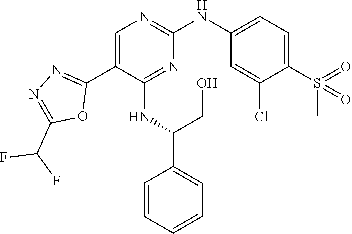 [SMILES: CS(=O)(=O)c1ccc(Nc2ncc(-c3nnc(C(F)F)o3)c(N[C@H](CO)c3ccccc3)n2)cc1Cl] | (2S)-2-[[2-(3-chloro-4- methylsulfonyl-anilino)-5-[5- (difluoromethyl)-1,3,4-oxadiazol-2- yl]pyrimidin-4-yl]amino]-2-phenyl- ethanol | 537.1 | 537 |
| 528 | 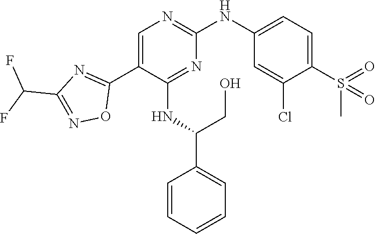 [SMILES: CS(=O)(=O)c1ccc(Nc2ncc(-c3nc(C(F)F)no3)c(N[C@H](CO)c3ccccc3)n2)cc1Cl] | (2S)-2-[[2-(3-chloro-4- methylsulfonyl-anilino)-5-[3- (difluoromethyl)-1,2,4-oxadiazol-5- yl]pyrimidin-4-l]amino]-2-phenyl- ethanol | 537.1 | 537 |
| 529 | 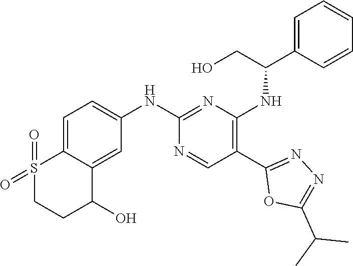 [SMILES: CC(C)c1nnc(-c2cnc(Nc3ccc4c(c3)C(O)CCS4(=O)=O)nc2N[C@H](CO)c2ccccc2)o1] | 6-[[4-[[(1S)-2-hydroxy-1-phenyl- ethyl]amino]-5-(5-isopropyl-1,3,4- oxadiazol-2-yl)pyrimidin-2- yl]amino]-1,1-dioxo-3,4-dihydro-2H- thiochromen-4-ol | 537.2 | 537 |
| 530 | 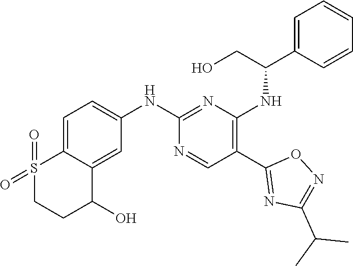 [SMILES: CC(C)c1noc(-c2cnc(Nc3ccc4c(c3)C(O)CCS4(=O)=O)nc2N[C@H](CO)c2ccccc2)n1] | 6-[[4-[[(1S)-2-hydroxy-1-phenyl- ethyl]amino]-5-(3-isopropyl-1,2,4- oxadiazol-5-yl)pyrimidin-2- yl]amino]-1,1-dioxo-3,4-dihydro-2H- thiochromen-4-ol | 537.2 | 537 |
| 531 | 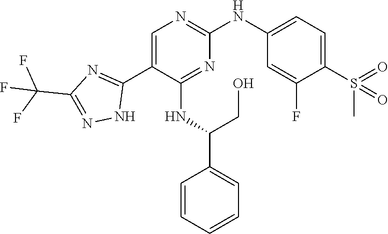 [SMILES: CS(=O)(=O)c1ccc(Nc2ncc(-c3nc(C(F)(F)F)n[nH]3)c(N[C@H](CO)c3ccccc3)n2)cc1F] | (2S)-2-[[2-(3-fluoro-4- methylsulfonyl-anilino)-5-[3- (trifluoromethyl)-1H-1,2,4-triazol-5- yl]pyrimidin-4-yl]amino]-2-phenyl- ethanol | 538.1 | 538 |
| 532 | 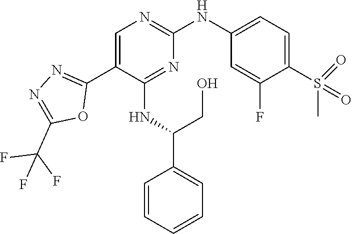 [SMILES: CS(=O)(=O)c1ccc(Nc2ncc(-c3nnc(C(F)(F)F)o3)c(N[C@H](CO)c3ccccc3)n2)cc1F] | (2S)-2-[[2-(3-fluoro-4- methylsulfonyl-anilino)-5-[5- (trifluoromethyl)-1,3,4-oxadiazol-2- yl]pyrimidin-4-yl]amino]-2-phenyl- ethanol | 539.1 | 539 |
| 533 | 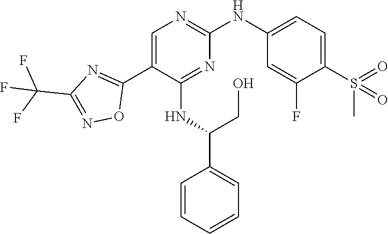 [SMILES: CS(=O)(=O)c1ccc(Nc2ncc(-c3nc(C(F)(F)F)no3)c(N[C@H](CO)c3ccccc3)n2)cc1F] | (2S)-2-[[2-(3-fluoro-4- methylsulfonyl-anlino)-5-[3- (trifluoromethyl)-1,2,4-oxadiazol-5- yl]pyrimidin-4-yl]amino]-2-phenyl- ethanol | 539.1 | 539 |
| 534 | 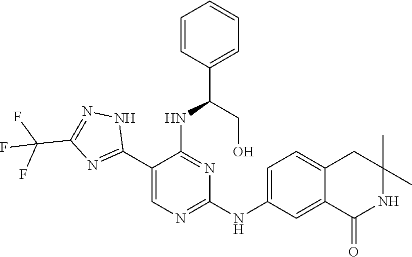 [SMILES: CC1(C)Cc2ccc(Nc3ncc(-c4nc(C(F)(F)F)n[nH]4)c(N[C@H](CO)c4ccccc4)n3)cc2C(=O)N1] | 7-[[4-[[(1S)-2-hydroxy-1-phenyl- ethyl]amino]-5-[3-(trifluoromethyl)- 1H-1,2,4-triazol-5-yl]pyrimidin-2- yl]amino]-3,3-dimethyl-2,4- dihydroisoquinolin-1-one | 539.2 | 539 |
| 535 | 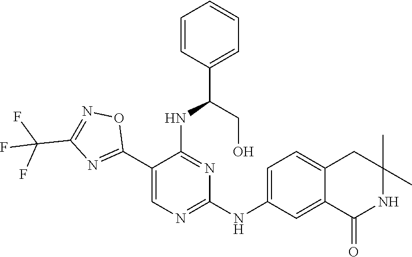 [SMILES: CC1(C)Cc2ccc(Nc3ncc(-c4nc(C(F)(F)F)no4)c(N[C@H](CO)c4ccccc4)n3)cc2C(=O)N1] | 7-[[4-[[(1S)-2-hydroxy-1-phenyl- ethyl]amino]-5-[3-(trifluoromethyl)- 1,2,4-oxadiazol-5-yl]pyrimidin-2- yl]amino]-3,3-dimethyl-2,4- dihydroisoquinolin-1-one | 540.2 | 540 |
| 536 | 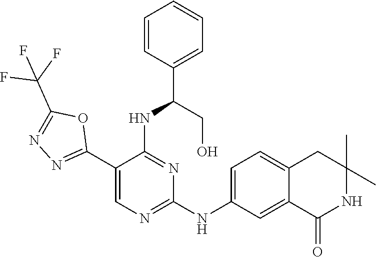 [SMILES: CC1(C)Cc2ccc(Nc3ncc(-c4nnc(C(F)(F)F)o4)c(N[C@H](CO)c4ccccc4)n3)cc2C(=O)N1] | 7-[[4-[[(1S)-2-hydroxy-1-phenyl- ethyl]amino]-5-[5-(trifluoromethyl)- 1,3,4-oxadiazol-2-yl]pyrimidin-2- yl]amino]-3,3-dimethyl-2,4- dihydroisoquinolin-1-one | 540.2 | 540 |
| 537 | 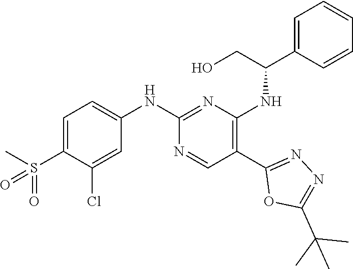 [SMILES: CC(C)(C)c1nnc(-c2cnc(Nc3ccc(S(C)(=O)=O)c(Cl)c3)nc2N[C@H](CO)c2ccccc2)o1] | (2S)-2-[[5-(5-tert-butyl-1,3,4- oxadiazol-2-yl)-2-(3-chloro-4- methysullfonyl-anilino)pyrimidin-4- yl]amino]-2-phenyl-ethanol | 543.2 | 543 |
| 538 | 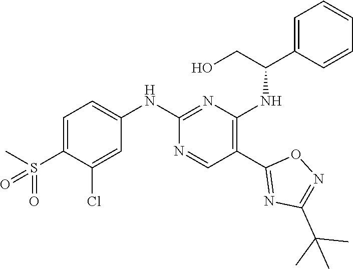 [SMILES: CC(C)(C)c1noc(-c2cnc(Nc3ccc(S(C)(=O)=O)c(Cl)c3)nc2N[C@H](CO)c2ccccc2)n1] | (2S)-2-[[5-(3-tert-butyl-1,2,4- oxadiazol-5-yl)-2-(3-chloro-4- methyslulfonyl-anilino)pyrimidin-4- yl]amino]-2-phenyl-ethanol | 543.2 | 543 |
| 539 | 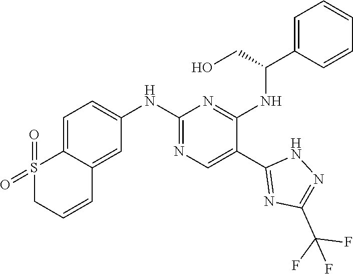 [SMILES: O=S1(=O)CC=Cc2cc(Nc3ncc(-c4nc(C(F)(F)F)n[nH]4)c(N[C@H](CO)c4ccccc4)n3)ccc21] | (2S)-2-[[2-[(1,1-dioxo-2H- thiochromen-6-yl)amino]-5-[3- (trifluoromethyl)-1H-1,2,4-triazol-5- yl]pyrimidin-4-yl]amino]-2-phenyl- ethanol | 544.1 | 544 |
| 540 | 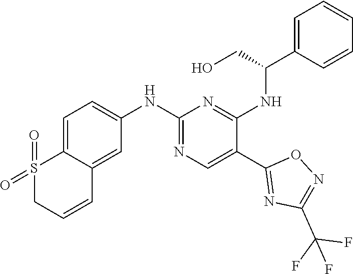 [SMILES: O=S1(=O)CC=Cc2cc(Nc3ncc(-c4nc(C(F)(F)F)no4)c(N[C@H](CO)c4ccccc4)n3)ccc21] | (2S)-2-[[2-[(1,1-dioxo-2H- thiochromen-6-yl)amino]-5-[3- (trifluoromethyl)-1,2,4-oxadiazol-5- yl]pyrimidin-4-yl]amino]-2-phenyl- ethanol | 545.1 | 545 |
| 541 | 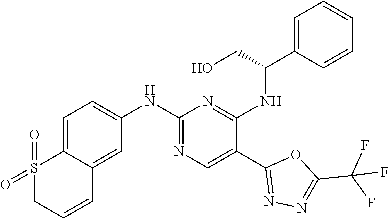 [SMILES: O=S1(=O)CC=Cc2cc(Nc3ncc(-c4nnc(C(F)(F)F)o4)c(N[C@H](CO)c4ccccc4)n3)ccc21] | (2S)-2-[[2-[(1,1-dioxo-2H- thiochromen-6-yl)amino]-5-[5- (trifluoromethyl)-1,3,4-oxadiazol-2- yl]pyrimidin-4-yl]amino]-2-phenyl- ethanol | 545.1 | 545 |
| 542 | 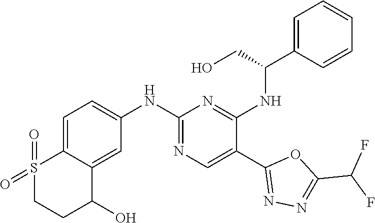 [SMILES: O=S1(=O)CCC(O)c2cc(Nc3ncc(-c4nnc(C(F)F)o4)c(N[C@H](CO)c4ccccc4)n3)ccc21] | 6-[[5-[5-(difluoromethyl)-1,3,4- oxadiazol-2-yl]-4-[[(1)S-2-hydroxy-1- phenyl-ethyl]amino]pyrimidin-2- yl]amino]-1,1-dioxo-3,4-dihydro-2H- thiochromen-4-ol | 545.1 | 545 |
| 543 | 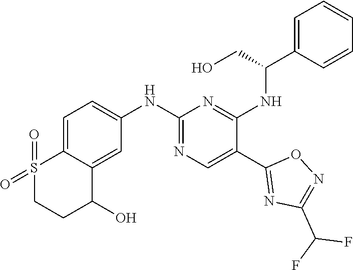 [SMILES: O=S1(=O)CCC(O)c2cc(Nc3ncc(-c4nc(C(F)F)no4)c(N[C@H](CO)c4ccccc4)n3)ccc21] | 6-[[5-[3-(difluoromethyl)-1,2,4- oxadiazol-5-yl]-4-[[(1S)-2-hydroxy-1- phenyl-ethyl]amino]pyrimidin-4- yl]amino]-1,1-dioxo-3,4-dihydro-2H- thiochromen-4-ol | 545.1 | 545 |
| 544 | 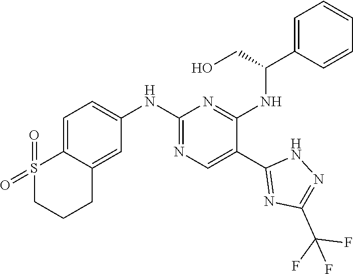 [SMILES: O=S1(=O)CCCc2cc(Nc3ncc(-c4nc(C(F)(F)F)n[nH]4)c(N[C@H](CO)c4ccccc4)n3)ccc21] | (2S)-2-[[2-[(1,1-dioxo-3,4-dihydro- 2H-thiochromen-6-yl)amino]-5-[3- (trifluoromethyl)-1H-1,2,4-triazol-5- yl]pyrimidin-4-yl]amino]-2-phenyl- ethanol | 546.2 | 546 |
| 545 | 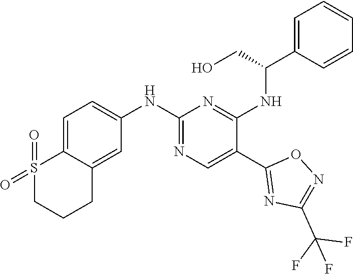 [SMILES: O=S1(=O)CCCc2cc(Nc3ncc(-c4nc(C(F)(F)F)no4)c(N[C@H](CO)c4ccccc4)n3)ccc21] | (2S)-2-[[2-[(1,1-dioxo-3,4-dihydroxy- 2H-thiochromen-6-yl)amino]-5-[3- (trifluoromethyl)-1,2,4-oxadiazol-5- yl]pyrimidin-4-yl]amino]-2-phenyl- ethanol | 547.1 | 547 |
| 546 | 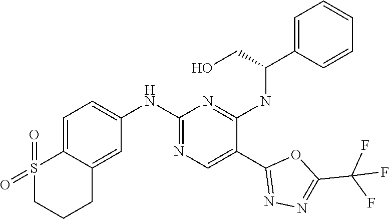 | (2S)-2-[[2-[(1,1-dioxo-3,4-dihydro- 2H-thiochromen-6-yl)amino]-5-[5- (trifluoromethyl)-1,3,4-oxadiazol-2- yl]pyrimidin-4-yl]amino]-2-phenyl- ethanol | 57.1 | 547 |
| 547 | 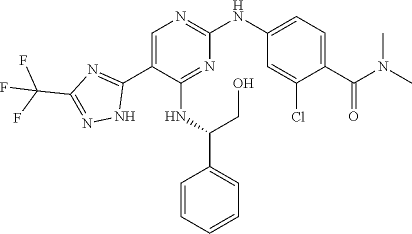 [SMILES: CN(C)C(=O)c1ccc(Nc2ncc(-c3nc(C(F)(F)F)n[nH]3)c(N[C@H](CO)c3ccccc3)n2)cc1Cl] | 2-chloro-4-[[4-[[(1S)-2-hydroxy-1- phenyl-ethyl]amino]-5-[3- (trifluoromethyl)-1H-1,2,4-triazol-5- yl]pyrimidin-2-yl]amino]-N,N- dimethyl-benzamide | 547.2 | 547 |
| 548 | 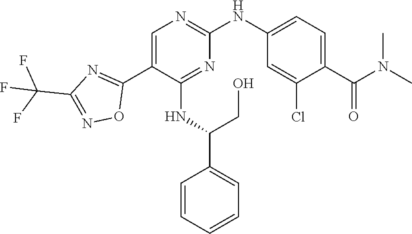 [SMILES: CN(C)C(=O)c1ccc(Nc2ncc(-c3nc(C(F)(F)F)no3)c(N[C@H](CO)c3ccccc3)n2)cc1Cl] | 2-chloro-4-[[4-[[(1S)-2-hydroxy-1- phenyl-ethyl]amino]-5-[3- (trifluoromethyl)-1,2,4-oxadiazol-5- yl]pyrimidin-2-yl]amino]-N,N- dimethyl-benzamide | 548.1 | 548 |
| 549 | 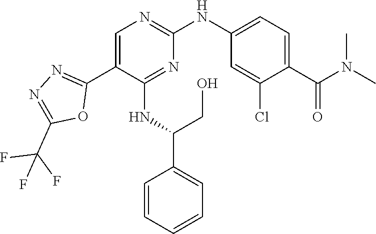 [SMILES: CN(C)C(=O)c1ccc(Nc2ncc(-c3nnc(C(F)(F)F)o3)c(N[C@H](CO)c3ccccc3)n2)cc1Cl] | 2-chloro-4-[[4-[[(1S)-2-hydroxy-1- phenyl-ethyl]amino]-5-[5- (trifluoromethyl)-1,3,4-oxadiazol-2- yl]pyrimidin-2-yl]amino]-N,N- dimethyl-benzamide | 548.1 | 548 |
| 550 | 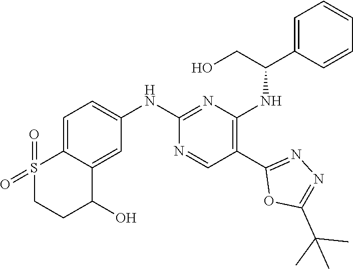 [SMILES: CC(C)(C)c1nnc(-c2cnc(Nc3ccc4c(c3)C(O)CCS4(=O)=O)nc2N[C@H](CO)c2ccccc2)o1] | 6-[[5-(5-tert-butyl-1,3,4-oxadiazol-2- yl)-4-[[(1S)-2-hydroxy-1-phenyl- ethyl]amino]pyrimidin-2-yl]amino]- 1,1-dioxo-3,4-dihydro-2H- thiochromen-4-ol | 551.2 | 551 |
| 551 | 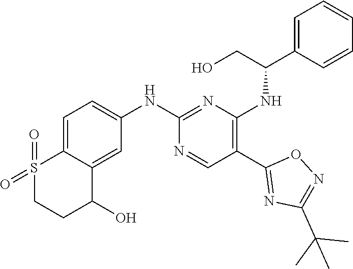 [SMILES: CC(C)(C)c1noc(-c2cnc(Nc3ccc4c(c3)C(O)CCS4(=O)=O)nc2N[C@H](CO)c2ccccc2)n1] | 6-[[5-(3-tert-butyl-1,2,4-oxadiazol-5- yl)-4-[[(1S)-2-hydroxy-1-phenyl- ethyl]amino]pyrimidin-2-yl]amino]- 1,1-dioxo-3,4-dihydro-2H- thiochromen-4-ol | 551.2 | 551 |
| 552 | 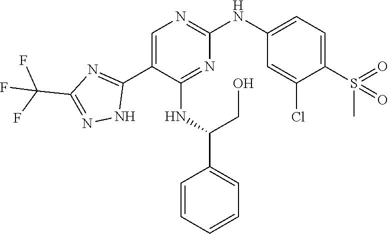 [SMILES: CS(=O)(=O)c1ccc(Nc2ncc(-c3nc(C(F)(F)F)n[nH]3)c(N[C@H](CO)c3ccccc3)n2)cc1Cl] | (2S)-2-[[2-(3-chloro-4- methylsulfonyl-anilino)-5-[3- (trifluoromethyl)-1H-1,2,4-triazol-5- yl]pyrimidin-4-yl]amino]-2-phenyl- ethanol | 554.1 | 554 |
| 553 | 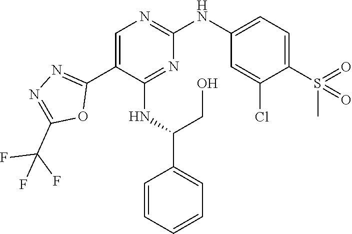 [SMILES: CS(=O)(=O)c1ccc(Nc2ncc(-c3nnc(C(F)(F)F)o3)c(N[C@H](CO)c3ccccc3)n2)cc1Cl] | (2S)-2-[[2-(3-chloro-4- methylsulfonyl-anilino)-5-[5- (trifluoromethyl)-1,3,4-oxadiazol-2- yl]pyrimidin-4-yl]amino]-2-phenyl- ethanol | 555.1 | 555 |
| 554 | 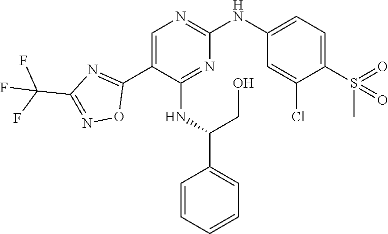 [SMILES: CS(=O)(=O)c1ccc(Nc2ncc(-c3nc(C(F)(F)F)no3)c(N[C@H](CO)c3ccccc3)n2)cc1Cl] | (2S)-2-[[2-(3-chloro-4- methyslulfonyl-anilino)-5-[3- (trifluoromethyl)-1,2,4-oxadiazol-5- yl]pyrimidin-4-yl]amino]-2-phenyl- ethanol | 555.1 | 555 |
| 555 | 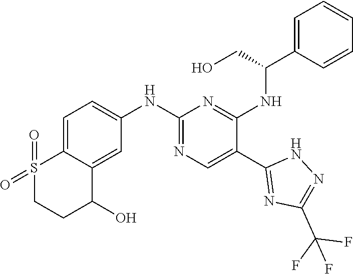 [SMILES: O=S1(=O)CCC(O)c2cc(Nc3ncc(-c4nc(C(F)(F)F)n[nH]4)c(N[C@H](CO)c4ccccc4)n3)ccc21] | 6-[[4-[[(1S)-2-hydroxy-1-phenyl- ethyl]amino]-5-[3-(trifluoromethyl)- 1H-1,2,4-triazol-5-yl]pyrimidin-2- yl]amino]-1,1-dioxo-3,4-dihydro-2H- thiochromen-4-ol | 562.1 | 562 |
| 556 | 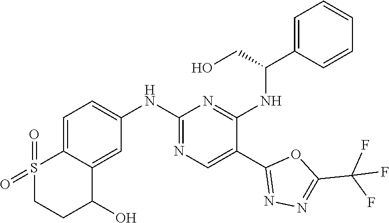 [SMILES: O=S1(=O)CCC(O)c2cc(Nc3ncc(-c4nnc(C(F)(F)F)o4)c(N[C@H](CO)c4ccccc4)n3)ccc21] | 6-[[4-[[(1S)-2-hydroxy-1-phenyl- ethyl]amino]-5-[5-(trifluoromethyl)- 1,3,4-oxadiazol-2-yl]pyrimidin-2- yl]amino]-1,1-dioxo-3,4-dihydro-2H- thiochromen-4-ol | 563.1 | 563 |
| 557 | 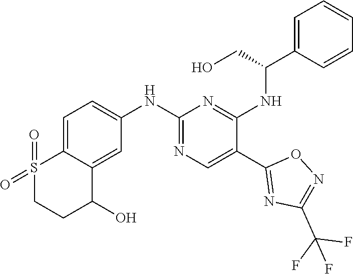 [SMILES: O=S1(=O)CCC(O)c2cc(Nc3ncc(-c4nc(C(F)(F)F)no4)c(N[C@H](CO)c4ccccc4)n3)ccc21] | 6-[[4-[[(1S)-2-hydroxy-1-phenyl- ethyl]amino]-5-[3-(trifluoromethyl)- 1,2,4-oxadiazol-5-yl]pyrimidin-2- yl]amino]-1,1-dioxo-3,4-dihydro-2H- thiochromen-4-ol | 563.1 | 563 |
| 558 | 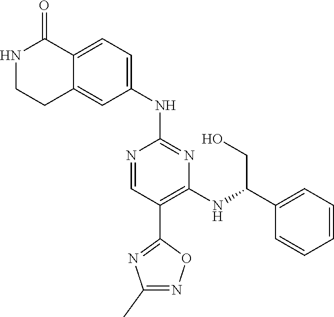 [SMILES: Cc1noc(-c2cnc(Nc3ccc4c(c3)CCNC4=O)nc2N[C@H](CO)c2ccccc2)n1] | 6-[[4-[[(1S)-2-hydroxy-1-phenyl- ethyl]amino]-5-(3-methyl-1,2,4- oxadiazol-5-yl)pyrimidin-2- yl]amino]-3,4-dihydro-2H- isoquinolin-1-one | 457.2 | 457 |
| 559 | 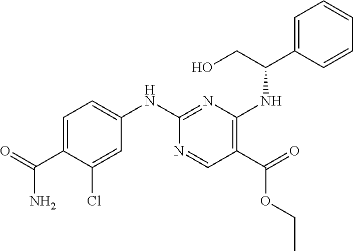 [SMILES: CCOC(=O)c1cnc(Nc2ccc(C(N)=O)c(Cl)c2)nc1N[C@H](CO)c1ccccc1] | ethyl 2-(4-carbamoyl-3-chloro- anilino)-4-[[(1S)-2-hydroxy-1-phenyl- ethyl]amino]pyrimidine-5-carboxylate | 455.1 | 455 |
| 560 | 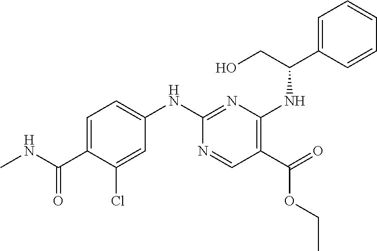 [SMILES: CCOC(=O)c1cnc(Nc2ccc(C(=O)NC)c(Cl)c2)nc1N[C@H](CO)c1ccccc1] | ethyl 2-[3-chloro-4- (methylcarbamoyl)anilino]-4-[[(1S)- 2-hydroxy-1-phenyl- ethyl]amino]pyrimidin-5-carboxylate | 469.2 | 469 |
| 561 | 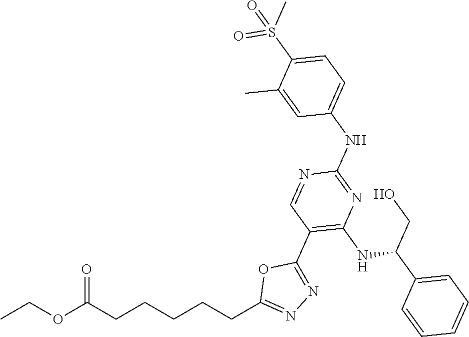 [SMILES: CCOC(=O)CCCCCc1nnc(-c2cnc(Nc3ccc(S(C)(=O)=O)c(C)c3)nc2N[C@H](CO)c2ccccc2)o1] | ethyl 6-[5-[4-[[(1S)-2-hydroxy-1- phenyl-ethyl]amino]-2-(3-methyl-4- methyslulfonyl-anilino)pyrimidin-5- yl]-1,3,4-oxadiazol-2-yl]hexanoate | 608.2 | 608 |
| 562 | 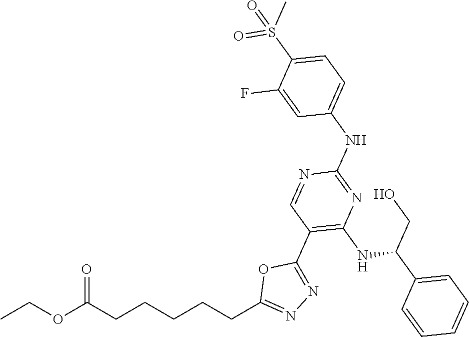 [SMILES: CCOC(=O)CCCCCc1nnc(-c2cnc(Nc3ccc(S(C)(=O)=O)c(F)c3)nc2N[C@H](CO)c2ccccc2)o1] | ethyl 6-[5-[2-(3-fluoro-4- methylsulfonyl-anilino)-4-[[(1S)-2- hydroxy-1-phenyl- ethyl]amino]pyrimidin-5-yl]-1,3,4- oxadiazol-2-yl]hexanoate | 612.2 | 612 |
| 563 | 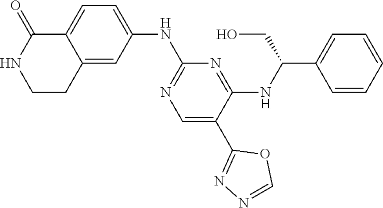 [SMILES: O=C1NCCc2cc(Nc3ncc(-c4nnco4)c(N[C@H](CO)c4ccccc4)n3)ccc21] | 6-[[4-[[(1S)-2-hydroxy-1-phenyl- ethyl]amino]-5-(1,3,4-oxadiazol-2- yl)pyrimidin-2-yl]amino]-3,4- dihydro-2H-isoquinolin-1-one | 444.2 | 444 |
| 564 | 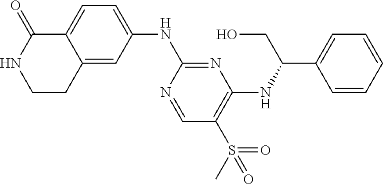 [SMILES: CS(=O)(=O)c1cnc(Nc2ccc3c(c2)CCNC3=O)nc1N[C@H](CO)c1ccccc1] | 6-[[4-[[(1S)-2-hydroxy-1-phenyl- ethyl]amino]-5-methylsulfonyl- pyrimidin-2-yl]amino]-3,4-dihydro- 2H-isoquinolin-1-one | 454.2 | 454 |
| 565 | 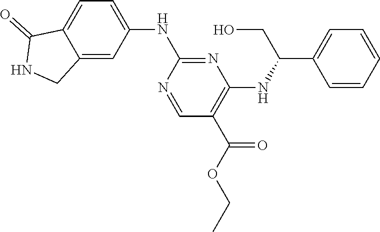 [SMILES: CCOC(=O)c1cnc(Nc2ccc3c(c2)CNC3=O)nc1N[C@H](CO)c1ccccc1] | ethyl 4-[[(1S)-2-hydroxy-1-phenyl- ethyl]amino]-2-[(1-oxoisoindolin-5- yl)amino]pyrimidine-5-carboxylate | 434.2 | 434 |
| 566 | 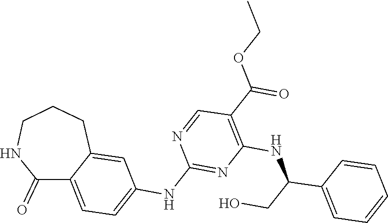 [SMILES: CCOC(=O)c1cnc(Nc2ccc3c(c2)CCCNC3=O)nc1N[C@H](CO)c1ccccc1] | ethyl 4-[[(1S)-2-hydroxy-1-phenyl- ethyl]amino]-2-[(1-oxo-2,3,4,5- tetrahydro-2-benzazepin-7- yl)amino]pyrimidine-5-carboxylate | 462.2 | 462 |
| 567 | 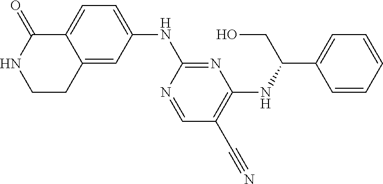 [SMILES: N#Cc1cnc(Nc2ccc3c(c2)CCNC3=O)nc1N[C@H](CO)c1ccccc1] | 4-[[(1S)-2-hydroxy-1-phenyl- ethyl]amino]-2-[(1-oxo-3,4-dihydro- 2H-isoquinolin-6- yl)amino]pyrimidine-5-carbonitrile | 401.2 | 401 |
| 568 | 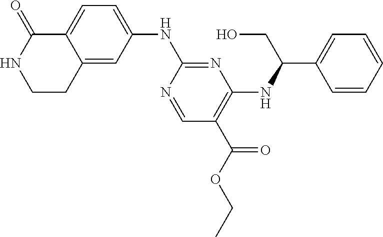 [SMILES: CCOC(=O)c1cnc(Nc2ccc3c(c2)CCNC3=O)nc1N[C@@H](CO)c1ccccc1] | ethyl 4-[[(1R)-2-hydroxy-1-phenyl- ethyl]amino]-2-[(1-oxo-3,4-dihydro- 2H-isoquinolin-6- yl)amino]pyrimidine-5-carobxylate | 448.2 | 448 |
| 569 | 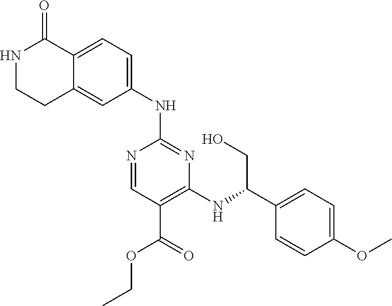 [SMILES: CCOC(=O)c1cnc(Nc2ccc3c(c2)CCNC3=O)nc1N[C@H](CO)c1ccc(OC)cc1] | ethyl 4-[[(1S)-2-hydroxy-1-(4- methoxyphenyl)ethyl]amino]-2-[(1- oxo-3,4-dihydro-2H-isoquinolin-6- yl)amino]pyrimidine-5-carboxylate | 478.2 | 478 |
| 570 | 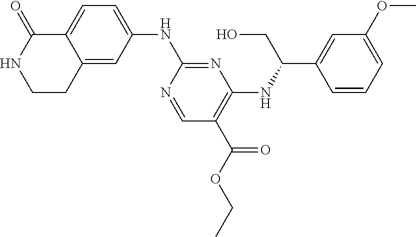 [SMILES: CCOC(=O)c1cnc(Nc2ccc3c(c2)CCNC3=O)nc1N[C@H](CO)c1cccc(OC)c1] | ethyl 4-[[(1S)-2-hydroxy-1-(3- methoxyphenyl)ethyl]amino]-2-[(1- oxo-3,4-dihydro-2H-isoquinolin-6- yl)amino]pyrimidine-5-carboxylate | 478.2 | 478 |
| 571 | 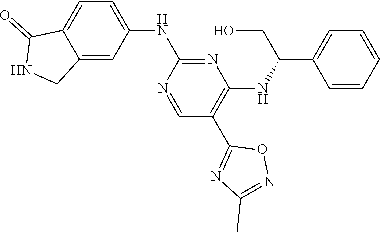 [SMILES: Cc1noc(-c2cnc(Nc3ccc4c(c3)CNC4=O)nc2N[C@H](CO)c2ccccc2)n1] | 5-[[4-[[(1S)-2-hydroxy-1-phenyl- ethyl]amino]-5-(3-methyl-1,2,4- oxadiazol-5-yl)pyrimidin-2- yl]amino]isoindolin-1-one | 444.2 | 444 |
| 572 | 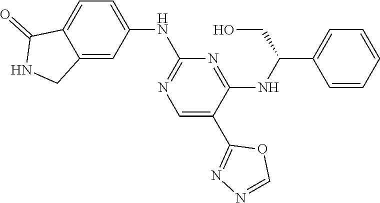 [SMILES: O=C1NCc2cc(Nc3ncc(-c4nnco4)c(N[C@H](CO)c4ccccc4)n3)ccc21] | 5-[[4-[[(1S)-2-hydroxy-1-phenyl- ethyl]amino]-5-(1,3,4-oxadiazol-2- yl)pyrimidin-2-yl]amino]isoindolin-1- one | 430.2 | 430 |
| 573 | 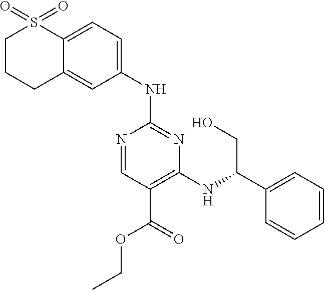 [SMILES: CCOC(=O)c1cnc(Nc2ccc3c(c2)CCCS3(=O)=O)nc1N[C@H](CO)c1ccccc1] | ethyl 2-[(1,1-dioxo-3,4-dihydro-2H- thiochromen-6-yl)amino]-4-[[(1S)-2- hydroxy-1-phenyl- ethyl]amino]pyrimidine-5-carboxylate | 483.2 | 483 |
| 574 | 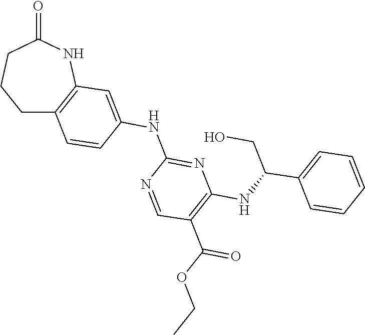 [SMILES: CCOC(=O)c1cnc(Nc2ccc3c(c2)NC(=O)CCC3)nc1N[C@H](CO)c1ccccc1] | ethyl 4-[[(1S)-2-hydroxy-1-phenyl- ethyl]amino]-2-[(2-oxo-1,3,4,5- tetrahydro-1-benzazepin-8- yl)amino]pyimidine-5-carboxylate | 462.2 | 462 |
| 575 | 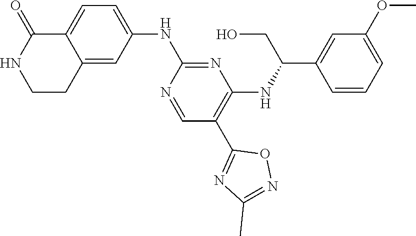 [SMILES: COc1cccc([C@@H](CO)Nc2nc(Nc3ccc4c(c3)CCNC4=O)ncc2-c2nc(C)no2)c1] | 6-[[4-[[(1S)-2-hydroxy-1-(3- methoxyphenyl)ethyl]amino]-5-(3- methyl-1,2,4-oxadiazol-5- yl)pyrimidin-2-yl]amino]-3,4- dihydro-2H-isoquinolin-1-one | 488.2 | 488 |
| 576 | 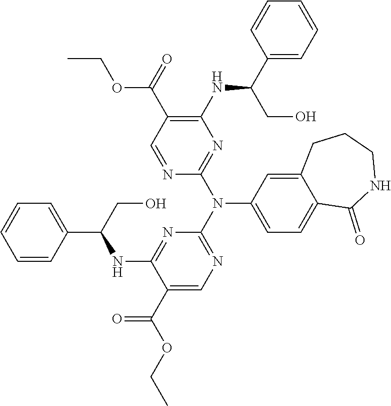 [SMILES: CCOC(=O)c1cnc(N(c2ccc3c(c2)CCCNC3=O)c2ncc(C(=O)OCC)c(N[C@H](CO)c3ccccc3)n2)nc1N[C@H](CO)c1ccccc1] | ethyl 2-[[5-ethoxycarbonyl-4-[[(1S)- 2-hydroxy-1-phenyl- ethyl]amino]pyrimidin-2-yl]-(1-oxo- 2,3,4,5-tetrahydro-2-benzazepin-7- yl)amino]-4-[[(1S)-2-hydroxy-1- phenyl-ethyl]amino]pyrimidin-5- carboxylate | 747.3 | 747 |
| 577 | 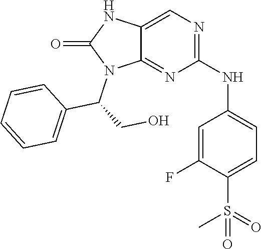 [SMILES: CS(=O)(=O)c1ccc(Nc2ncc3[nH]c(=O)n([C@H](CO)c4ccccc4)c3n2)cc1F] | 2-(3-fluoro-4-methylsulfonyl- anilino)-9-[(1S)-2-hydroxy-1-phenyl- ethyl]-7H-purin-8-one | 444.1 | 444 |
| 578 | 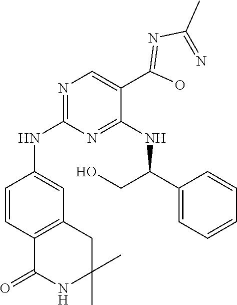 [SMILES: CC(=N)/N=C(\O)c1cnc(Nc2ccc3c(c2)CC(C)(C)NC3=O)nc1N[C@H](CO)c1ccccc1] | 6-[[4-[[(1S)-2-hydroxy-1-phenyl- ethyl]amino]-5-(3-methyl-1,2,4- oxadiazol-5-yl)pyrimidin-2- yl]amino]-3,3-dimethyl-2,4- dihydroisoquinolin-1-one | 486.2 | 486 |
| 579 | 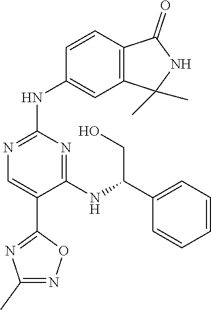 [SMILES: Cc1noc(-c2cnc(Nc3ccc4c(c3)C(C)(C)NC4=O)nc2N[C@H](CO)c2ccccc2)n1] | 5-[[4-[[(1S)-2-hydroxy-1-phenyl- ethyl]amino]-5-(3-methyl-1,2,4- oxadiazol-5-yl)pyrimidin-2- yl]amino]-3,3-dimethyl-isoindolin-1- one | 472.2 | 472 |
| 580 | 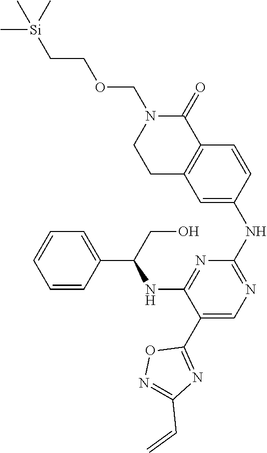 | 6-[[4-[[(1S)-2-hydroxy-1-phenyl- ethyl]amino]-5-(3-vinyl-1,2,4- oxadiazol-5-yl)pyrimidin-2- yl]amino]-2-(2- trimethylsilylethoxymethyl)-3,4- dihydroisoquinolin-1-one | 600.3 | 600 |
| 581 | 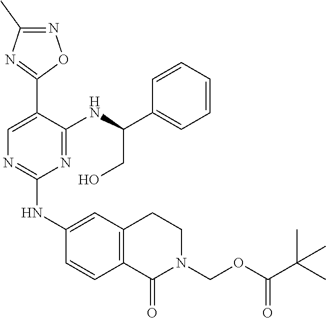 [SMILES: Cc1noc(-c2cnc(Nc3ccc4c(c3)CCN(COC(=O)C(C)(C)C)C4=O)nc2N[C@H](CO)c2ccccc2)n1] | [6-[[4-[[(1S)-2-hydroxy-1-phenyl- ethyl]amino]-5-(3-methyl-1,2,4- oxadiazol-5-l)pyrimidin-2- yl]amino]-1-oxo-3,4- dihydroisoquinolin-2-yl]methyl 2,2- dimethylpropanoate | 572.3 | 572 |
| 582 | 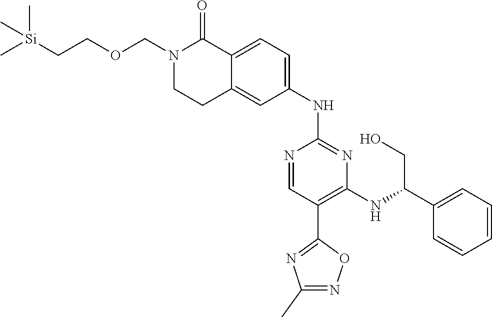 [SMILES: Cc1noc(-c2cnc(Nc3ccc4c(c3)CCN(COCC[Si](C)(C)C)C4=O)nc2N[C@H](CO)c2ccccc2)n1] | 6-[[4-[[(1S)-2-hydroxy-1-phenyl- ethyl]amino]-5-(3-methyl-1,2,4- oxadiazol-5-yl)pyrimidin-2- yl]amino]-2-(2- trimethylsilylethoxymethyl)-3,4- dihydroisoquinolin-1-one | 588.3 | 588 |
| 583 | 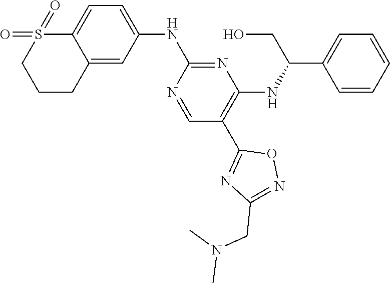 [SMILES: CN(C)Cc1noc(-c2cnc(Nc3ccc4c(c3)CCCS4(=O)=O)nc2N[C@H](CO)c2ccccc2)n1] | (2S)-2-[[5-[3- [(dimethylamino)methyl]-1,2,4- oxadiazol-5-yl]-2-[(1,1-dioxo-3,4- dihydro-2H-thiochromen-6- yl)amino]pyrimidin-4-yl]amino]-2- phenyl-ethanol | 536.2 | 536 |
| 584 | 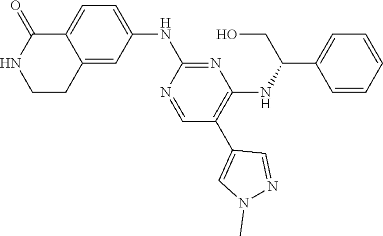 [SMILES: Cn1cc(-c2cnc(Nc3ccc4c(c3)CCNC4=O)nc2N[C@H](CO)c2ccccc2)cn1] | 6-[[4-[[(1S)-2-hydroxy-1-phenyl- ethyl]amino]-5-(1-methylpyrazol-4- yl)pyrimidin-2-yl]amino]-3,4- dihydro-2H-isoquinolin-1-one | 456.2 | 456 |
| 585 | 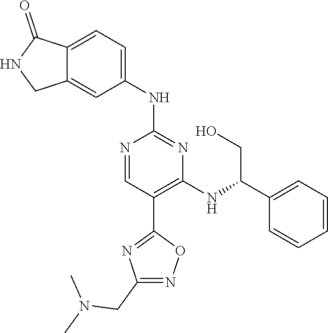 [SMILES: CN(C)Cc1noc(-c2cnc(Nc3ccc4c(c3)CNC4=O)nc2N[C@H](CO)c2ccccc2)n1] | 5-[[5-[3-[(dimethylamino)methyl]- 1,2,4-oxadiazol-5-yl]-4-[[(1S)-2- hydroxy-1-phenyl- ethyl]amino]pyrimidin-2- yl]amino]isoindolin-1-one | 487.2 | 487 |
| 586 | 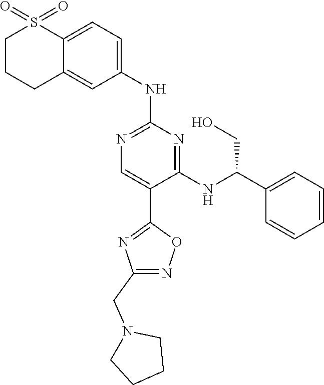 [SMILES: O=S1(=O)CCCc2cc(Nc3ncc(-c4nc(CN5CCCC5)no4)c(N[C@H](CO)c4ccccc4)n3)ccc21] | (2S)-2-[[2-[(1,1-dioxo-3,4-dihydro- 2H-thiochromen-6-yl)amino]-5-[3- (pyrrolidin-1-ylmethyl)-1,2,4- oxadiazol-5-yl]pyrimidin-4- yl]amino]-2-phenyl-ethanol | 562.2 | 562 |
| 587 | 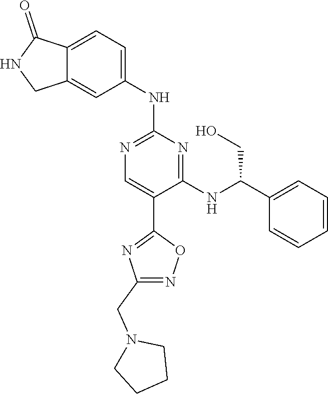 [SMILES: O=C1NCc2cc(Nc3ncc(-c4nc(CN5CCCC5)no4)c(N[C@H](CO)c4ccccc4)n3)ccc21] | 5-[[4-[[(1S)-2-hydroxy-1-phenyl- ethyl]amino]-5-[3-(pyrrolidin-1- ylmethyl)-1,2,4-oxadiazol-5- yl]pyrimidin-2-yl]amino]isoindolin-1- one | 513.2 | 513 |

### General Synthetical Procedures and Examples of the Compound's Preparation.

### Preparation of Monochlorides.

### Preparation 1: Ethyl 2-chloro-4-{[(1S)-2-hydroxy-1-phenylethyl]amino}-pyrimidine-5-carboxylate

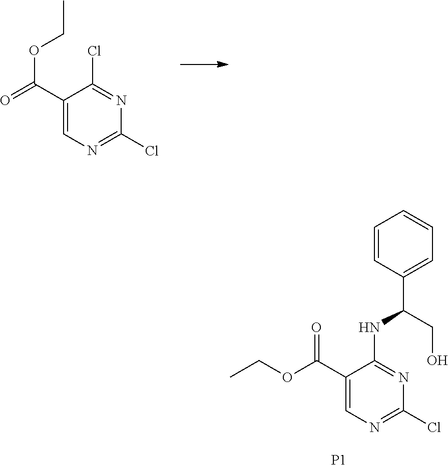 [SMILES: CCOC(=O)c1cnc(Cl)nc1Cl ; CCOC(=O)c1cnc(Cl)nc1N[C@H](CO)c1ccccc1 ; "P1"]

A mixture of ethyl 2,4-dichloropyrimidine-5-carboxylate (5.50 g, 24.9 mmol), (S)-2-amino-2-phenylethan-1-ol (3.75 g, 27.3 mmol), DIPEA (3.52 g, 27.3 mmol) in MeCN (70 mL) was stirred for 3 h at ambient temperature and concentrated under reduced pressure. The residue was subjected to silica CC eluting with a mixture EtOAc and n-hexane (1:2) to afford (6.25 g, 78%) of title compound as a white solid. ¹H NMR (400 MHz, DMSO-d₆): δ 9.06 (d, J=8.2 Hz, 1H), 8.65 (s, 1H), 7.36-7.24 (m, 5H), 5.25-5.18 (m, 2H), 4.35 (q, J=7.0 Hz, 2H), 3.82-3.70 (m, 2H), 1.34 (t, J=7.0 Hz, 3H).

### Preparation 2: Ethyl 2-chloro-4-{[(1R)-2-hydroxy-1-phenylethyl]amino}-pyrimidine-5-carboxylate

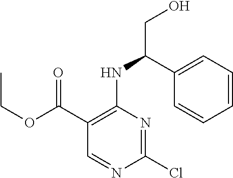 [SMILES: CCOC(=O)c1cnc(Cl)nc1N[C@@H](CO)c1ccccc1]

The compound was synthesized according to the procedure described in [symbol] using (2R)-2-amino-2-phenylethanol instead of (S)-2-amino-2-phenylethan-1-ol. Product was analyzed by LCMS: [MH⁺] 324, 322, 323.

### Preparation 3: Ethyl 2-chloro-4-{[(1R)-1-phenylethyl]amino}pyrimidine-5-carboxylate

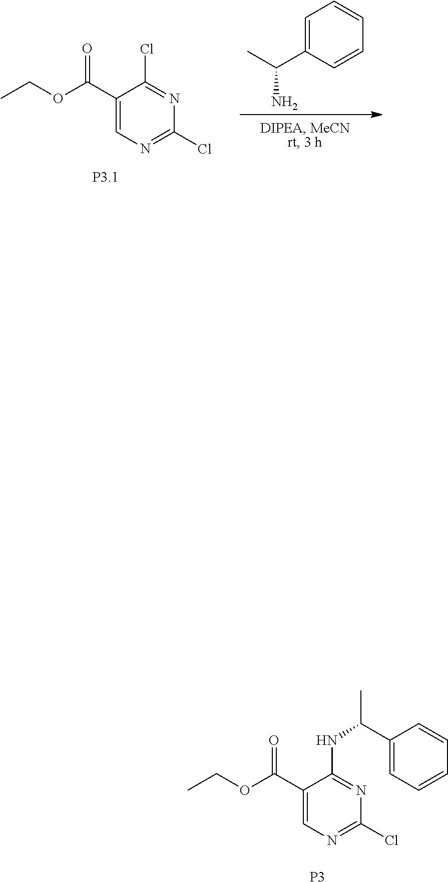

To a solution of ethyl 2,4-dichloropyrimidine-5-carboxylate P3.1 (100 mg, 0.452 mmol) in MeCN (5 mL) a (R)-1-phenyl-ethylamine (60 mg, 0.497 mmol) and DIPEA (64 mg, 0.497 mmol) was added at room temperature. After the reaction was stirred for 3 h, the mixture was concentrated to give a residue. The residue was purified by silica gel column chromatography (EtOAc:n-hexane=1:2) to give P3 (54 mg, 39% yield). The product was analyzed by LCMS.

### Preparation 4: Ethyl 2-chloro-4-{[(1S,2R)-2-hydroxy-2,3-dihydro-1H-inden-1-yl]amino}pyrimidine-5-carboxylate

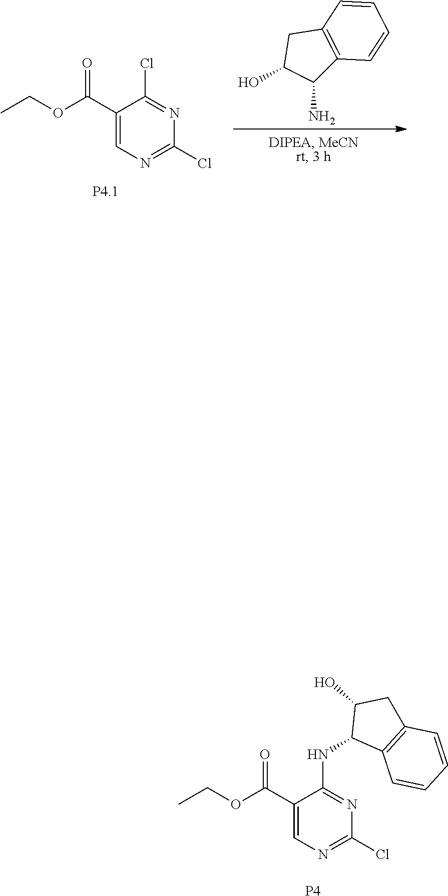

To a solution of ethyl 2,4-dichloropyrimidine-5-carboxylate P4.1 (100 mg, 0.452 mmol) in MeCN (5 mL) a (1S,2R)-1-amino-2-indanol (74 mg, 0.497 mmol) and DIPEA (64 mg, 0.497 mmol) was added at room temperature. After the reaction was stirred for 3 h, the mixture was concentrated to give a residue. The residue was purified by silica gel column chromatography (EtOAc:n-hexane=1:2) to give P4 (112 mg, 51% yield). The product was analyzed by LCMS.

### Preparation 5: Ethyl 2-chloro-4-{[(1S,2S)-2-hydroxy-2,3-dihydro-1H-inden-1-yl]amino}pyrimidine-5-carboxylate

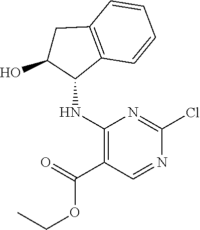 [SMILES: CCOC(=O)c1cnc(Cl)nc1N[C@H]1c2ccccc2C[C@@H]1O]

The compound was synthesized according to the procedure described in [symbol] using (1S,2S)-1-amino-2,3-dihydro-1H-inden-2-ol instead of (S)-2-amino-2-phenylethan-1-ol. The product was analyzed by LCMS.

### Preparation 6: Ethyl 2-chloro-4-{[(1S)-1-cyclohexyl-2-hydroxyethyl]amino}-pyrimidine-5-carboxylate

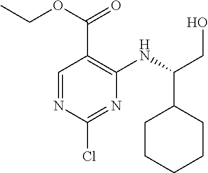 [SMILES: CCOC(=O)c1cnc(Cl)nc1N[C@H](CO)C1CCCCC1]

The compound was synthesized according to the procedure described in [symbol] using (2S)-2-amino-2-cyclohexylethanol instead of (S)-2-amino-2-phenylethan-1-ol. The product was analyzed by LCMS.

### Preparation 7: Ethyl 4-{[(1S)-1-benzyl-2-hydroxyethyl]amino}-2-chloropyrimidine-5-carboxylate

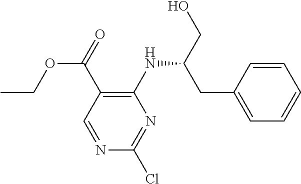 [SMILES: CCOC(=O)c1cnc(Cl)nc1N[C@H](CO)Cc1ccccc1]

The compound was synthesized according to the procedure described in [symbol] using (2S)-2-amino-3-phenylpropan-1-ol instead of (S)-2-amino-2-phenylethan-1-ol. The product was analyzed by LCMS.

### Preparation 8: Ethyl 2-chloro-4-{[(1S)-1-(4-chlorophenyl)-2-hydroxyethyl]-amino}pyrimidine-5-carboxylate

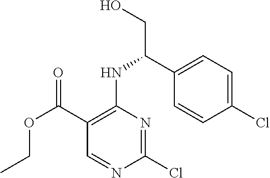 [SMILES: CCOC(=O)c1cnc(Cl)nc1N[C@H](CO)c1ccc(Cl)cc1]

The compound was synthesized according to the procedure described in [symbol] using (2S)-2-amino-2-(4-chlorophenyl)ethanol instead of (S)-2-amino-2-phenylethan-1-ol. The product was analyzed by LCMS.

### Preparation 9: Ethyl 2-chloro-4-[(2-fluoro-1-phenylethyl)amino]pyrimidine-5-carboxylate

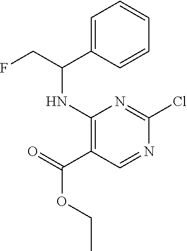 [SMILES: CCOC(=O)c1cnc(Cl)nc1NC(CF)c1ccccc1]

The compound was synthesized according to the procedure described in [symbol] using 2-fluoro-1-phenylethanamine instead of (S)-2-amino-2-phenylethan-1-ol. The product was analyzed by LCMS.

### Preparation 10: Ethyl 2-chloro-4-{[(1R,2S)-2-hydroxy-2,3-dihydro-1H-inden-1-yl]amino}pyrimidine-5-carboxylate

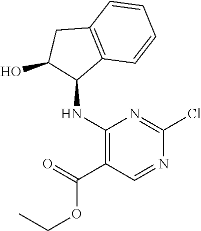 [SMILES: CCOC(=O)c1cnc(Cl)nc1N[C@@H]1c2ccccc2C[C@@H]1O]

The compound was synthesized according to the procedure described in [symbol] using (1R,2S)-1-amino-2,3-dihydro-1H-inden-2-ol instead of (S)-2-amino-2-phenylethan-1-ol. The product was analyzed by LCMS.

### Preparation 11: Ethyl 2-chloro-4-{[(1S)-2-hydroxy-1-(pyridin-3-ylmethyl)ethyl]amino}pyrimidine-5-carboxylate

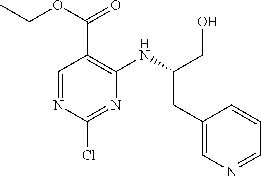 [SMILES: CCOC(=O)c1cnc(Cl)nc1N[C@H](CO)Cc1cccnc1]

The compound was synthesized according to the procedure described in [symbol] using (2S)-2-amino-3-pyridin-3-ylpropan-1-ol instead of (S)-2-amino-2-phenylethan-1-ol. The product was analyzed by LCMS.

### Preparation 12: Ethyl 2-chloro-4-[3-(hydroxymethyl)-3,4-dihydro-1H-isoquinolin-2-yl]pyrimidine-5-carboxylate

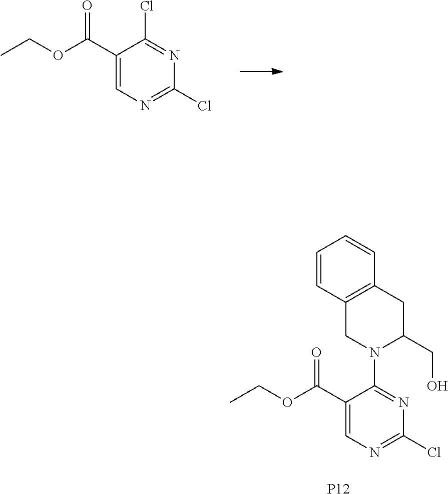 [SMILES: CCOC(=O)c1cnc(Cl)nc1Cl ; CCOC(=O)c1cnc(Cl)nc1N1Cc2ccccc2CC1CO ; "P12"]

To a solution of ethyl 2,4-dichloropyrimidine-5-carboxylate (100 mg, 0.452 mmol) in MeCN (5 mL) a 3-hydroxymethyl-1,2,3,4-tetrahydroisoquinoline (81 mg, 0.497 mmol) and DIPEA (64 mg, 0.497 mmol) was added at room temperature. After the reaction was stirred for 3 h, the mixture was concentrated to give a residue. The residue was purified by silica gel column chromatography (EtOAc:n-hexane=1:2) to give 2 (50 mg, 32% yield). A product was analyzed only with LCMS.

### Preparation 13: ethyl 2-chloro-4-[(2S)-2-(hydroxymethyl)-2,3-dihydro-1H-indol-1-yl]pyrimidine-5-carboxylate

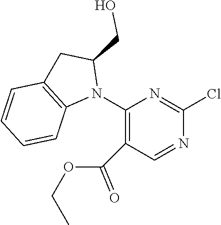 [SMILES: CCOC(=O)c1cnc(Cl)nc1N1c2ccccc2C[C@H]1CO]

The compound was synthesized according to the procedure described in [symbol] using (2S)-2,3-dihydro-1H-indol-2-ylmethanol instead of (S)-2-amino-2-phenylethan-1-ol. The product was analyzed by LCMS.

### Preparation 14: ethyl 2-chloro-4-{[(1S)-2-hydroxy-1-(1H-indol-3-ylmethyl)ethyl]amino}pyrimidine-5-carboxylate

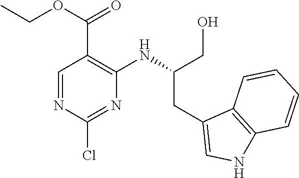 [SMILES: CCOC(=O)c1cnc(Cl)nc1N[C@H](CO)Cc1c[nH]c2ccccc12]

The compound was synthesized according to the procedure described in [symbol] using (2S)-2-amino-3-(1H-indol-3-yl)propan-1-ol instead of (S)-2-amino-2-phenylethan-1-ol. The product was analyzed by LCMS.

### Preparation 15: (2S)-2-[(2-chloro-5-nitro-pyrimidin-4-yl)amino]-2-phenyl-ethanol

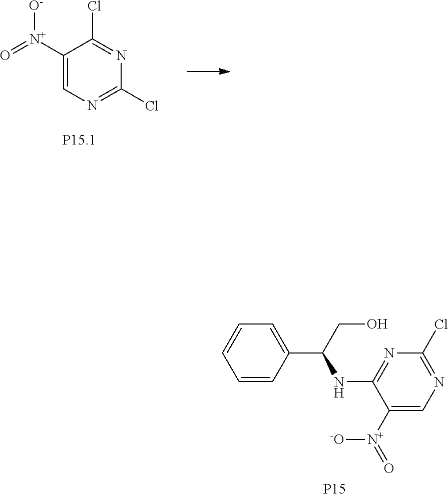

To a solution of 5-nitro-2,4-dichloropyrimidine (P15.1, 1.0 g, 5.15 mmol) in MeCN (10 mL) a (S)-2-amino-2-phenylethan-1-ol (0.77 g, 5.67 mmol) and DIPEA (0.73 g, 5.67 mmol) was added at room temperature. After the reaction was stirred for 3 h, the mixture was concentrated to give a residue. The residue was purified by silica gel column chromatography (DCM:Et₂O=4:1) to give P15 (1.1 g, 73% yield). ¹H NMR (400 MHz, DMSO-d₆): δ 9.15 (d, J=7.8 Hz, 1H), 9.05 (s, 1H), 7.43-7.41 (m, 2H), 7.34 (t, J=7.8 Hz, 2H), 7.29-7.25 (m, 1H), 5.38-5.33 (m, 1H), 5.29-5.16 (m, 1H), 3.88-3.80 (m, 4H).

### Preparation of Esters by Substitution of the Second Chlorine.

### Preparation 16: Ethyl 4-{[(1S)-2-hydroxy-1-phenylethyl]amino}-2-{[3-methyl-4-(methylsulfonyl)phenyl]amino}pyrimidine-5-carboxylate

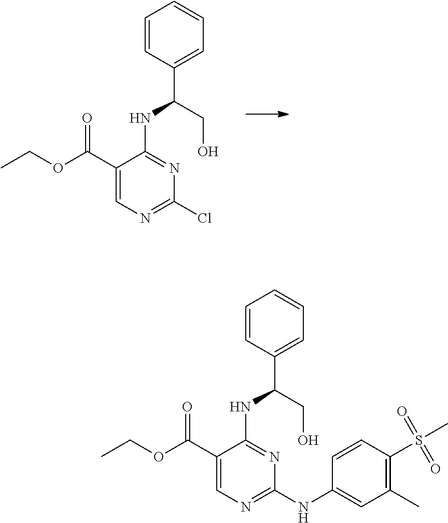 [SMILES: CCOC(=O)c1cnc(Cl)nc1N[C@H](CO)c1ccccc1 ; CCOC(=O)c1cnc(Nc2ccc(S(C)(=O)=O)c(C)c2)nc1N[C@H](CO)c1ccccc1]

A mixture of ethyl 2-chloro-4-{[(1S)-2-hydroxy-1-phenylethyl]amino}pyrimidine-5-carboxylate (See [symbol] ) (0.70 g, 2.17 mmol), 4-sulfonylmethyl-3-methylaniline (0.44 g, 2.39 mmol), dioxane (3 mL) and p-TsOH hydrate (0.45 g, 2.39 mmol) was heated under microwave irradiation at 110° C. for a 30 min, diluted with EtOAc (10 mL), washed with 10% aq. solution of NaHCO₃, brine, dried over Na₂SO₄, and concentrated under reduced pressure. The residue was subjected to silica CC eluting with a mixture of EtOAc and n-hexane (1:1) to afford 0.78 g (76%) of the title compound as a white solid. ¹H NMR (400 MHz, DMSO-d₆): δ 10.12 (s, 1H), 9.01 (d, J=8.2 Hz, 1H), 8.64 (s, 1H), 7.72-7.64 (m, 2H), 7.38-7.21 (m, 5H), 5.23-5.19 (m, 2H), 4.33 (q, J=7.0 Hz, 2H), 3.88-3.68 (m, 2H), 3.14 (s, 3H), 2.54 (s, 3H), 1.34 (t, J=7.0 Hz, 3H).

### Preparation 17: Ethyl 4-{[(1R)-2-hydroxy-1-phenylethyl]amino}-2-{[3-methyl-4-(methylsulfonyl)phenyl]amino}pyrimidine-5-carboxylate (Compound 169)

The compound was synthesized according to the procedure described in [symbol] using ethyl 2-chloro-4-{[(1R)-2-hydroxy-1-phenylethyl]amino}pyrimidine-5-carboxylate (See [symbol] ) instead of ethyl 2-chloro-4-{[(1S)-2-hydroxy-1-phenylethyl]amino}pyrimidine-5-carboxylate. The product was analyzed by LCMS.

### Preparation 18: Ethyl 2-{[3-methyl-4-(methylsulfonyl)phenyl]amino}-4-{[(1R)-1-phenylethyl]amino}pyrimidine-5-carboxylate (Compound 64)

The compound was synthesized according to the procedure described in [symbol] using ethyl 2-chloro-4-{[(1R)-1-phenylethyl]amino}pyrimidine-5-carboxylate (See [symbol] ) instead of ethyl 2-chloro-4-{[(1S)-2-hydroxy-1-phenylethyl]amino}-pyrimidine-5-carboxylate. The product was analyzed by LCMS.

### Preparation 19: Ethyl 4-{[(1S,2R)-2-hydroxy-2,3-dihydro-1H-inden-1-yl]amino}-2-{[3-methyl-4-(methylsulfonyl)phenyl]amino}pyrimidine-5-carboxylate (Compound 260)

The compound was synthesized according to the procedure described in [symbol] using (1S,2R)-1-amino-2,3-dihydro-1H-inden-2-ol (See [symbol] ) instead of ethyl 2-chloro-4-{[(1S)-2-hydroxy-1-phenylethyl]amino}pyrimidine-5-carboxylate. The product was analyzed by LCMS.

### Preparation 20: Ethyl 4-{[(1S,2S)-2-hydroxy-2,3-dihydro-1H-inden-1-yl]amino}-2-{[3-methyl-4-(methylsulfonyl)phenyl]amino}pyrimidine-5-carboxylate (Compound 261)

The compound was synthesized according to the procedure described in [symbol] using (1S,2S)-1-amino-2,3-dihydro-1H-inden-2-ol (See [symbol] ) instead of ethyl 2-chloro-4-{[(1S)-2-hydroxy-1-phenylethyl]amino}pyrimidine-5-carboxylate. The product was analyzed by LCMS.

### Preparation 21: Ethyl 4-{[(1S)-1-cyclohexyl-2-hydroxyethyl]amino}-2-{[3-methyl-4-(methylsulfonyl)phenyl]amino}pyrimidine-5-carboxylate (Compound 222)

The compound was synthesized according to the procedure described in [symbol] using (2S)-2-amino-2-cyclohexylethanol (See [symbol] ) instead of ethyl 2-chloro-4-{[(1S)-2-hydroxy-1-phenylethyl]amino}pyrimidine-5-carboxylate. The product was analyzed by LCMS.

### Preparation 22: Ethyl 4-{[(1S)-1-benzyl-2-hydroxyethyl]amino}-2-{[3-methyl-4-(methylsulfonyl)phenyl]amino}pyrimidine-5-carboxylate (Compound 273)

The compound was synthesized according to the procedure described in [symbol] using ethyl 4-{[(1S)-1-benzyl-2-hydroxyethyl]amino}-2-chloropyrimidine-5-carboxylate (See [symbol] ) instead of ethyl 2-chloro-4-{[(1S)-2-hydroxy-1-phenylethyl]amino}pyrimidine-5-carboxylate. The product was analyzed by LCMS.

### Preparation 23: Ethyl 4-{[(1S)-1-(4-chlorophenyl)-2-hydroxyethyl]amino}-2-{[3-methyl-4-(methylsulfonyl)phenyl]amino}pyrimidine-5-carboxylate (Compound 403)

The compound was synthesized according to the procedure described in [symbol] using Ethyl 2-chloro-4-{[(1S)-1-(4-chlorophenyl)-2-hydroxyethyl]amino}pyrimidine-5-carboxylate (See [symbol] ) instead of ethyl 2-chloro-4-{[(1S)-2-hydroxy-1-phenylethyl]amino}pyrimidine-5-carboxylate. The product was analyzed by LCMS.

### Preparation 24: Ethyl 4-[(2-fluoro-1-phenylethyl)amino]-2-{[3-methyl-4-(methylsulfonyl)phenyl]amino}pyrimidine-5-carboxylate (Compound 194)

The compound was synthesized according to the procedure described in [symbol] using Ethyl 2-chloro-4-[(2-fluoro-1-phenylethyl)amino]pyrimidine-5-carboxylate (See [symbol] ) instead of Ethyl 2-chloro-4-{[(1S)-2-hydroxy-1-phenylethyl]amino}pyrimidine-5-carboxylate. The product was analyzed by LCMS.

### Preparation 25: Ethyl 4-{[(1R,2S)-2-hydroxy-2,3-dihydro-1H-inden-1-yl]amino}-2-{[3-methyl-4-(methylsulfonyl)phenyl]amino}pyrimidine-5-carboxylate (Compound 262)

The compound was synthesized according to the procedure described in [symbol] using ethyl 2-chloro-4-{[(1R,2S)-2-hydroxy-2,3-dihydro-1H-inden-1-yl]amino}pyrimidine-5-carboxylate (See [symbol] ) instead of Ethyl 2-chloro-4-{[(1S)-2-hydroxy-1-phenylethyl]amino}pyrimidine-5-carboxylate. The product was analyzed by LCMS.

### Preparation 26: Ethyl 4-{[(1S)-2-hydroxy-1-(pyridin-3-ylmethyl)ethyl]amino}-2-{[3-methyl-4-(methylsulfonyl)phenyl]amino}pyrimidine-5-carboxylate (Compound 281)

The compound was synthesized according to the procedure described in [symbol] using ethyl 2-chloro-4-{[(1S)-2-hydroxy-1-(pyridin-3-ylmethyl)ethyl]amino}pyrimidine-5-carboxylate (See Preparation 11) instead of Ethyl 2-chloro-4-{[(1S)-2-hydroxy-1-phenylethyl]amino}pyrimidine-5-carboxylate. The product was analyzed by LCMS.

### Preparation 27: Ethyl 4-[(2S)-2-(hydroxymethyl)-2,3-dihydro-1H-indol-1-yl]-2-{[3-methyl-4-(methylsulfonyl)phenyl]amino}pyrimidine-5-carboxylate (Compound 263)

The compound was synthesized according to the procedure described in [symbol] using ethyl 2-chloro-4-[(2S)-2-(hydroxymethyl)-2,3-dihydro-1H-indol-1-yl]pyrimidine-5-carboxylate (See [symbol] ) instead of Ethyl 2-chloro-4-{[(1S)-2-hydroxy-1-phenylethyl]amino}pyrimidine-5-carboxylate. The product was analyzed by LCMS.

### Preparation 28: Ethyl 4-{[(1S)-2-hydroxy-1-(1H-indol-3-ylmethyl)ethyl]amino}-2-{[3-methyl-4-(methylsulfonyl)phenyl]amino}pyrimidine-5-carboxylate (Compound 491)

The compound was synthesized according to the procedure described in [symbol] using ethyl 2-chloro-4-{[(1S)-2-hydroxy-1-(1H-indol-3-ylmethyl)ethyl]amino}pyrimidine-5-carboxylate ( [symbol] [symbol] ) instead of Ethyl 2-chloro-4-{[(1S)-2-hydroxy-1-phenylethyl]amino}pyrimidine-5-carboxylate. The product was analyzed by LCMS.

### Preparation 29: Ethyl 2-{[3-fluoro-4-(methylsulfonyl)phenyl]amino}-4-{[(1S)-2-hydroxy-1-phenylethyl]amino}pyrimidine-5-carboxylate (Compound 210)

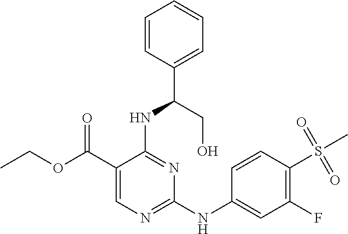 [SMILES: CCOC(=O)c1cnc(Nc2ccc(S(C)(=O)=O)c(F)c2)nc1N[C@H](CO)c1ccccc1]

To a solution of ethyl 2-chloro-4-{[(1S)-2-hydroxy-1-phenylethyl]amino}pyrimidine-5-carboxylate ( [symbol] ; 1.0 g, 0.0031 mol) in Dioxane (20 ml) was added 3-fluoro-4-(methylsulfonyl)aniline (0.59 g, 0.0031 mol) and TsOH (0.59 g, 0.0031 mmol). The reaction mixture was stirred at 100° C. for 30 min under microwave irradiation, cooled down to rt, and concentrated in vacuo. The residue was dissolved in EtOAc (20 ml), washed with water solution NaHCO₃—5% and dried over sodium sulfate. The organic layer was evaporated in vacuo and the crude product was purified by column chromatography (eluting with DCM/Et₂O) to provide Compound 210 as a solid. Yield is 1.4 g, 95%. ¹H-NMR (400 MHz, dmso-d₆) δ: 10.38 (br s, 1H), 9.08 (d, J=7.2 Hz, 1H), 8.67 (s, 1H), 7.83-7.74 (m, 1H), 7.65 (t, J=8.2 Hz, 1H), 7.49 (d, J=9.8 Hz, 1H), 7.38 (d, J=8.0 Hz, 2H), 7.33 (t, J=7.2 Hz, 2H), 7.33 (t, J=6.1 Hz, 1H), 5.27-5.17 (m, 2H), 4.32 (q, J=7.6 Hz, 2H), 3.91-3.80 (m, 1H), 3.76-3.66 (m, 1H), 3.26 (s, 3H), 1.34 (t, J=7.1 Hz, 3H).

### Preparation 30: Ethyl 2-{[3-fluoro-4-(methylsulfonyl)phenyl]amino}-4-{[(1R)-1-phenylethyl]amino}pyrimidine-5-carboxylate—Compound 97

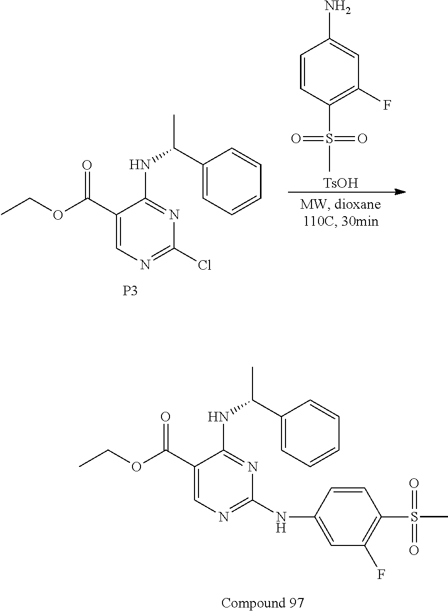

To a solution of P3 (54 mg, 0.177 mmol) and 4-sulfonylmethyl-3-fluoroaniline (34 mg, 0.177 mmol) in a vial in dioxane (3 ml) a TsOH-H₂O (34 mg, 0.177 mmol) was added. A tube was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel column chromatography (EtOAc:n-hexane=1:1) to give Compound 97 (29 mg, 36% yield). ¹H NMR (400 MHz, DMSO-d₆): δ 10.42 (s, 1H), 8.68-8.67 (m, 2H), 7.86 (d, J=13.8 Hz, 1H), 7.68 (t, J=8.0 Hz, 1H), 7.55 (dd, J₁=8.9 Hz, J₂=1.5 Hz, 1H), 7.42-7.40 (m, 2H), 7.36 (t, J=8.0 Hz, 2H), 7.26-7.23 (m, 1H), 5.35-5.28 (m, 1H), 4.30 (q, J=7.0 Hz, 2H), 3.25 (s, 3H), 1.57 (d, J=7.0 Hz, 3H), 1.32 (t, J=7.0 Hz, 3H).

### : Ethyl 4-{[(1S,2R)-2-hydroxy-2,3-dihydro-1H-inden-1-yl]amino}-2-{[3-fluoro-4-(methylsulfonyl)phenyl]amino}pyrimidine-5-carboxylate—Compound 298

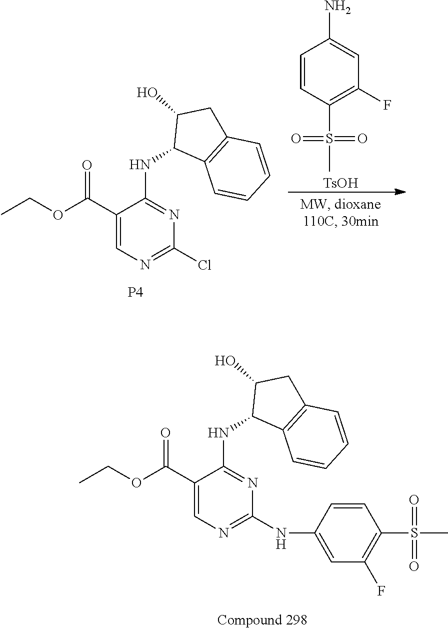

To a solution of P4 (112 mg, 0.335 mmol) and 4-sulfonylmethyl-3-fluoroaniline (64 mg, 0.335 mmol) in a vial in dioxane (3 ml) a TsOH-H₂O (64 mg, 0.335 mmol) was added. The vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel column chromatography (EtOAc:n-hexane=1:1) to give Compound 298 (75 mg, 46% yield). ¹H NMR (400 MHz, DMSO-d₆): δ 10.48 (s, 1H), 8.98 (d, J=8.0 Hz, 1H), 8.72 (s, 1H), 8.03 (d, J=13.6 Hz, 1H), 7.71-7.63 (m, 2H), 7.31-7.30 (m, 1H), 7.25-7.21 (m, 2H), 7.16 (d, J=7.0 Hz, 1H), 5.65-5.62 (m, 1H), 4.61 (t, J=4.8 Hz, 1H), 4.26 (q, J=7.0 Hz, 2H), 3.83-3.80 (m, 1H), 3.22-3.16 (m, 4H), 2.90 (d, J=16.4 Hz, 1H), 1.30 (t, J=7.0 Hz, 3H).

### Preparation 32: Ethyl 4-{[(1S)-1-benzyl-2-hydroxyethyl]amino}-2-{[3-fluoro-4-(methylsulfonyl)phenyl]amino}pyrimidine-5-carboxylate

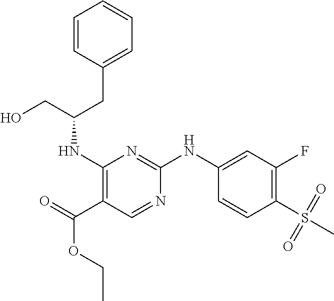 [SMILES: CCOC(=O)c1cnc(Nc2ccc(S(C)(=O)=O)c(F)c2)nc1N[C@H](CO)Cc1ccccc1]

The compound was synthesized according to the procedure described in [symbol] using ethyl 4-{[(1S)-1-benzyl-2-hydroxyethyl]amino}-2-chloropyrimidine-5-carboxylate (See [symbol] ) instead of ethyl 2-chloro-4-{[(1S)-2-hydroxy-1-phenylethyl]amino}pyrimidine-5-carboxylate and 3-fluoro-4-(methylsulfonyl)aniline instead of 3-methyl-4-(methylsulfonyl)aniline. LCMS: [MH⁺] 489.

### Preparation 33: Ethyl 4-[(2-fluoro-1-phenylethyl)amino]-2-{[3-fluoro-4-(methylsulfonyl)phenyl]amino}pyrimidine-5-carboxylate

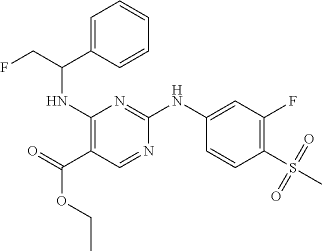 [SMILES: CCOC(=O)c1cnc(Nc2ccc(S(C)(=O)=O)c(F)c2)nc1NC(CF)c1ccccc1]

The compound was synthesized according to the procedure described in Preparation 16 using Ethyl 2-chloro-4-[(2-fluoro-1-phenylethyl)amino]pyrimidine-5-carboxylate (See [symbol] ) instead of ethyl 2-chloro-4-{[(1S)-2-hydroxy-1-phenylethyl]amino}pyrimidine-5-carboxylate and 3-fluoro-4-(methylsulfonyl)aniline instead of 3-methyl-4-(methylsulfonyl)aniline. LCMS: [MH⁺] 477.

### Preparation 34: Ethyl 4-{[(1S)-2-hydroxy-1-(pyridin-3-ylmethyl)ethyl]amino}-2-{[3-fluoro-4-(methylsulfonyl)phenyl]amino}pyrimidine-5-carboxylate

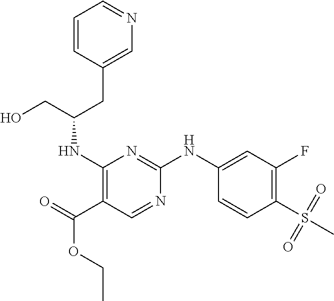 [SMILES: CCOC(=O)c1cnc(Nc2ccc(S(C)(=O)=O)c(F)c2)nc1N[C@H](CO)Cc1cccnc1]

The compound was synthesized according to the procedure described in [symbol] using Ethyl 2-chloro-4-{[(1S)-2-hydroxy-1-(pyridin-3-ylmethyl)ethyl]amino}pyrimidine-5-carboxylate (See [symbol] ) instead of Ethyl 2-chloro-4-{[(1S)-2-hydroxy-1-phenylethyl]amino}pyrimidine-5-carboxylate and 3-fluoro-4-(methylsulfonyl)aniline instead of 3-methyl-4-(methylsulfonyl)aniline. LCMS: [MH⁺] 490.

### Preparation 35: Ethyl 2-(3-fluoro-4-methylsulfonyl-anilino)-4-[3-(hydroxymethyl)-3,4-dihydro-1H-isoquinolin-2-yl]pyrimidine-5-carboxylate—Compound 388

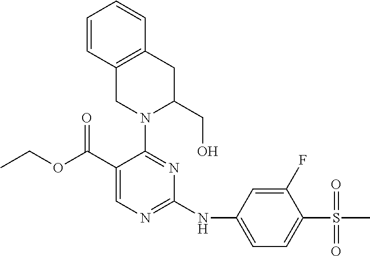 [SMILES: CCOC(=O)c1cnc(Nc2ccc(S(C)(=O)=O)c(F)c2)nc1N1Cc2ccccc2CC1CO]

To a solution of P12 (50 mg, 0.144 mmol) and 4-sulfonylmethyl-3-fluoroaniline (27 mg, 0.144 mmol) in a vial in dioxane (3 ml) a TsOH-H₂O (27 mg, 0.144 mmol) was added. The vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel column chromatography (EtOAc:n-hexane=1:1) to give Compound 388 (12 mg, 17% yield). ¹H NMR (400 MHz, DMSO-d₆): δ 10.83 (s, 1H), 8.87 (s, 1H), 7.96 (d, J=13.1 Hz, 1H), 7.83 (t, J=8.4 Hz, 1H), 7.73 (dd, J1=8.7 Hz, J2=1.6 Hz, 1H), 7.32-7.28 (m, 4H), 4.88-4.84 (m, 1H), 4.73-4.68 (m, 1H), 4.47-4.38 (m, 2H), 4.25 (q, J=7.0 Hz, 2H), 4.12-4.08 (m, 1H), 3.25 (m, 1H), 3.28 (s, 3H), 3.21-3.19 (m, 2H), 1.28 (t, J=7.0 Hz, 3H).

### Preparation 36: Ethyl 4-[(2S)-2-(hydroxymethyl)-2,3-dihydro-1H-indol-1-yl]-2-{[3-fluoro-4-(methylsulfonyl)phenyl]amino}pyrimidine-5-carboxylate

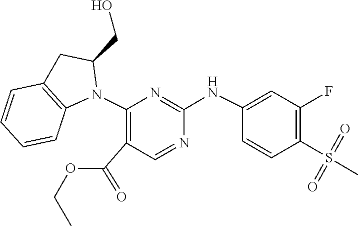 [SMILES: CCOC(=O)c1cnc(Nc2ccc(S(C)(=O)=O)c(F)c2)nc1N1c2ccccc2C[C@H]1CO]

The compound was synthesized according to the procedure described in [symbol] using ethyl 2-chloro-4-[(2S)-2-(hydroxymethyl)-2,3-dihydro-1H-indol-1-yl]pyrimidine-5-carboxylate (See [symbol] ) instead of ethyl 2-chloro-4-{[(1S)-2-hydroxy-1-phenylethyl]amino}pyrimidine-5-carboxylate and 3-fluoro-4-(methylsulfonyl)aniline instead of 3-methyl-4-(methylsulfonyl)aniline. LCMS: [MH⁺] 487.

### Preparation 37: Ethyl 4-{[(1S)-2-hydroxy-1-(1H-indol-3-ylmethyl)ethyl]amino}-2-{[3-fluoro-4-(methylsulfonyl)phenyl]amino}pyrimidine-5-carboxylate

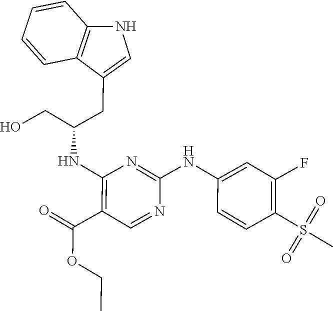 [SMILES: CCOC(=O)c1cnc(Nc2ccc(S(C)(=O)=O)c(F)c2)nc1N[C@H](CO)Cc1c[nH]c2ccccc12]

The compound was synthesized according to the procedure described in [symbol] using ethyl 2-chloro-4-{[(1S)-2-hydroxy-1-(1H-indol-3-ylmethyl)ethyl]amino}pyrimidine-5-carboxylate (See [symbol] ) instead of ethyl 2-chloro-4-{[(1S)-2-hydroxy-1-phenylethyl]amino}pyrimidine-5-carboxylate and 3-fluoro-4-(methylsulfonyl)aniline instead of 3-methyl-4-(methylsulfonyl)aniline. LCMS: [MH⁺] 528.

### Preparation 38: Ethyl 4-{[(1S)-2-hydroxy-1-phenylethyl]amino}-2-[(1-oxo-1,2,3,4-tetrahydroisoquinolin-6-yl)amino]pyrimidine-5-carboxylate (Compound 45)

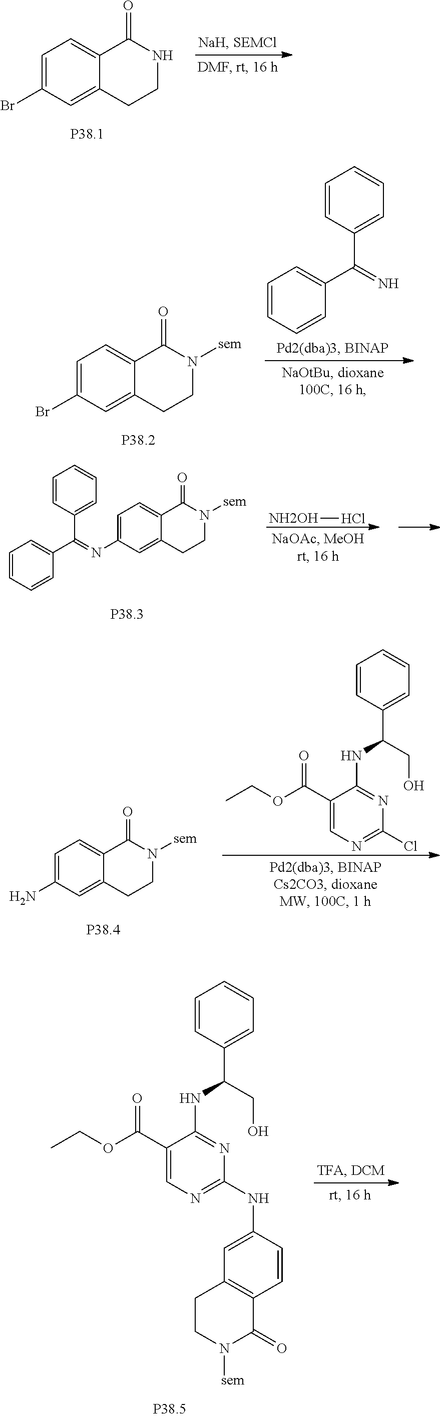

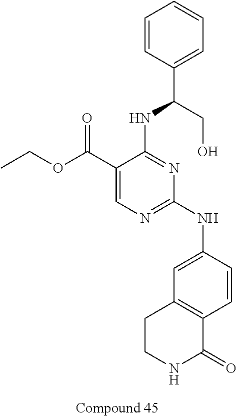 [SMILES: CCOC(=O)c1cnc(Nc2ccc3c(c2)CCNC3=O)nc1N[C@H](CO)c1ccccc1]

Synthesis of P38.2. To a solution of compound P38.1 (1.50 g, 6.63 mmol) in DMF (50 ml) a 60% of NaH (0.290 g, 7.30 mmol) was added and mixture stirred at rt for a 30 min, then a SEMCl (1.21 g, 7.30 mmol) was added, and mixture was stirred at rt for a 16 h. A mixture was poured in water (200 ml) and extracted with Et₂O (2×30 ml), organic layer was washed with water, dried under Na₂SO₄, and concentrated to give a residue. The residue was purified by silica gel column chromatography (DCM:Et₂O=10:1) to give P38.2 (0.710 g, 30% yield). ¹H NMR (400 MHz, DMSO-d₆): δ 7.97 (d, J=8.2 Hz, 1H), 7.59-7.55 (m, 2H), 4.89 (s, 2H), 3.46 (t, J=6.4 Hz, 2H), 3.50 (t, J=7.9 Hz, 2H), 2.98 (t, J=6.4 Hz, 2H), 0.87 (t, J=7.8 Hz, 2H), 0.00 (s, 9H).

Synthesis of P38.3. To a mixture P38.2 (0.710 g, 1.99 mmol) and 1,1-diphenylmethanimine (0.360 g, 2.00 mmol) in dioxane (20 ml) a NaO^{t}Bu (0.380 g, 4.00 mmol) was added, and a flow of argon passed through solution. A Pd₂(dba)₃ (91.5 mg, 0.100 mmol) and BINAP (124.0 mg, 0.200 mmol) was added, and mixture was refluxed under argon for a 16 h. Then a mixture was diluted with water (20 ml) and EtOAc (100 ml), organic layer was separated, aqueous was extracted with EtOAc (50 ml). Combined organic layer was washed with water, dried under Na₂SO₄, and concentrated to give a residue. The residue was purified by silica gel column chromatography (DCM:Et₂O=10:1) to give P38.3 (0.34 g, 34% yield) and P38.4 (0.290 g, 46% yield). ¹H NMR (400 MHz, DMSO-d₆): δ 7.66-7.62 (m, 3H), 7.57-7.53 (m, 1H), 7.50-7.45 (m, 2H), 7.34-7.32 (m, 3H), 7.18-7.16 (m, 2H), 6.66-6.65 (m, 1H), 6.61 (dd, J1=8.3 Hz, J2=2.6 Hz, 1H), 4.84 (s, 2H), 3.49-3.45 (m, 4H), 2.81 (t, J=6.4 Hz, 2H), 0.85 (t, J=7.9 Hz, 2H), 0.00 (s, 9H).

Synthesis of P38.4. To a solution of compound P38.3 (0.310 g, 0.200 mmol) and hydroxylamine hydrochloride (0.100 g, 1.40 mmol) in MeOH (5 ml) a NaOAc (0.172 g, 2.0 mmol) was added, and mixture was stirred at rt for a 16 h. Then a mixture was diluted with 1% solution of NaOH and extracted with DCM (2×10 ml). Combined organic layer was washed with water, dried under Na₂SO₄, and concentrated to give a residue. The residue was purified by silica gel column chromatography (DCM:EtOAc=2:1) to give P38.4 (0.120 g, 60% yield).). ¹H NMR (400 MHz, DMSO-d₆): δ 7.56 (d, J=8.4 Hz, 1H), 6.45 (dd, J1=8.4 Hz, J2=2.4 Hz, 1H), 6.32 (d, J=1.8 Hz, 1H), 5.74 (br, 2H), 4.83 (s, 2H), 3.49-3.44 (m, 4H), 2.77 (t, J=6.6 Hz, 2H), 0.86 (t, J=7.9 Hz, 2H), 0.00 (s, 9H).

Synthesis of P38.5. To a mixture of compound P38.4 (0.400 g, 1.37 mmol) and ethyl 2-chloro-4-{[(1S)-2-hydroxy-1-phenylethyl]amino}pyrimidine-5-carboxylate (0.440 g, 1.37 mmol, Preparation 1) in dioxane (10 ml) in a vial a Cs₂CO₃ (0.890 g, 2.74 mmol) was added, and a flow of argon passed through solution. A Pd(OAc)₂ (31 mg, 0.14 mmol) and BINAP (169 mg, 0.28 mmol) was added, a vial was capped and irradiated at 100° C. for a 1 h. Then a mixture was diluted with water (20 ml) and EtOAc (10 ml), organic layer was separated, aqueous was extracted with EtOAc (10 ml). Combined organic layer was washed with water, dried under Na₂SO₄, and concentrated to give a residue. The residue was purified by silica gel column chromatography (DCM:EtOAc=2:1) to give P38.5 (0.550 g, 70% yield). ¹H NMR (400 MHz, DMSO-d₆): δ 10.01 (s, 1H), 8.99 (d, J=7.5 Hz, 1H), 8.63 (s, 1H), 7.73 (d, J=8.7 Hz, 1H), 7.54-7.52 (m, 2H), 7.37-7.31 (m, 4H), 7.24-7.20 (m, 1H), 5.29-5.24 (m, 1H), 5.15 (t, J=4.8 Hz, 1H), 4.88 (s, 2H), 4.31 (q, J=6.7 Hz, 2H), 3.87-3.82 (m, 1H), 3.75-3.72 (m, 1H), 3.56-3.49 (m, 4H), 2.94-2.84 (m, 2H), 1.34 (t, J=7.1 Hz, 3H), 0.88 (t, J=7.5 Hz, 2H), 0.00 (s, 9H).

Synthesis of Compound 45. To a solution of compound P38.5 (100 mg, 0.17 mmol) in DCM (1 ml) a TFA (0.2 ml) was added, and mixture was stirred at rt for a 16 h. Then solution was diluted with 10% solution of NaHCO₃, organic layer was separated, washed with water, dried under Na₂SO₄, and concentrated to give a residue. The residue was purified by HPLC to give Compound 45 (5 mg, 6% yield). ¹H NMR (400 MHz, DMSO-d₆): δ 9.96 (s, 1H), 9.00 (d, J=7.5 Hz, 1H), 8.62 (s, 1H), 7.70-7.62 (m, 2H), 7.52-7.50 (m, 2H), 7.37-7.31 (m, 4H), 7.24-7.21 (m, 1H), 5.29-5.24 (m, 1H), 5.15 (t, J=4.9 Hz, 1H), 4.30 (q, J=7.0 Hz, 2H), 3.86-3.70 (m, 2H), 2.88-2.76 (m, 2H), 1.97-1.64 (m, 2H), 1.33 (t, J=7.0 Hz, 3H).

### Preparation 39: Ethyl 2-[(6-fluorobiphenyl-3-yl)amino]-4-{[(1S)-2-hydroxy-1-phenylethyl]amino}pyrimidine-5-carboxylate (Compound 195)

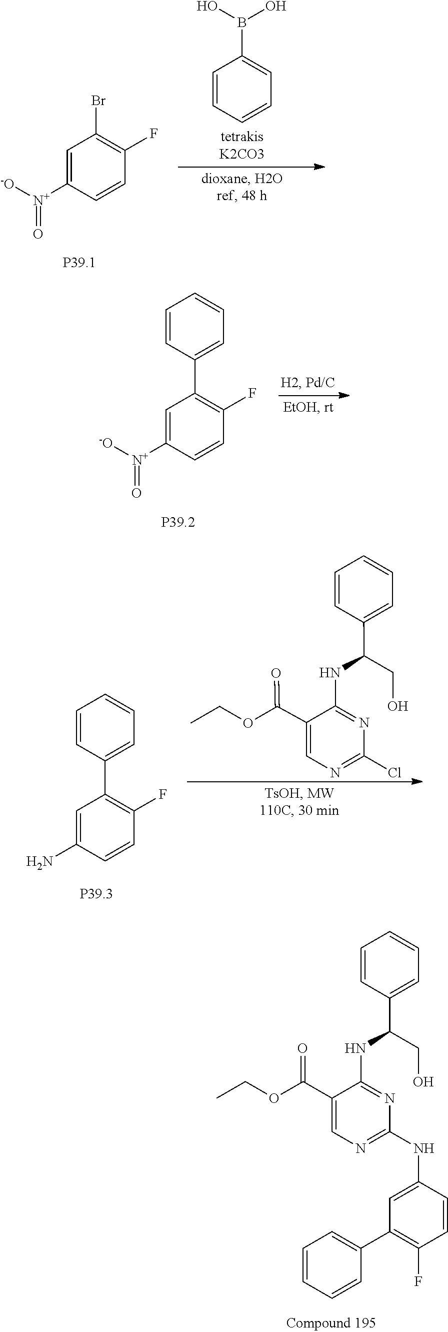

Synthesis of P39.2. To a mixture of 2-fluoro-5-nitro-bromobenzene P39.1 (2.00 g, 9.09 mmol) and boronic acid (1.66 g, 13.60 mmol) in dioxane (20 ml) a solution of K₂CO₃ (2.50 g, 18.18 mmol) in water (5 ml) was added and a flow of argon passed through solution. A tetrakis (0.50 g, 0.45 mmol) was added and mixture was refluxed under argon for a 48 h. Then the mixture was diluted with water (20 ml) and EtOAc (100 ml), organic layer was separated, aqueous was extracted with EtOAc (50 ml). Combined organic layer was washed with water, brine, dried under Na₂SO₄ and concentrated to give a residue. The residue was purified by silica gel column chromatography (EtOAc:n-hexane=1:10) to give P39.2 (1.57 g, 77% yield) as a slight-orange solid. ¹H NMR (400 MHz, DMSO-d₆): δ 8.35-8.29 (m, 2H), 7.66-7.61 (m, 3H), 7.56-7.47 (m, 3H).

Synthesis of P39.3. To a solution of P39.2 (1.52 g, 7.00 mmol) in EtOH (20 ml) in autoclave a 10% Pd/C (0.15 g) was added, and mixture was stirred under H₂ (5 bar) at rt for a 16 h. Then a mixture was filtered through a pad of Celite, and filtrate was concentrated to give P39.3 (1.05 g, 80% yield) as a slight-yellow solid. ¹H NMR (400 MHz, DMSO-d₆): δ 7.46-7.34 (m, 5H), 6.97-6.91 (m, 1H), 6.66-6.63 (m, 1H), 6.56-6.53 (m, 1H), 5.02 (br., 2H).

Synthesis of Compound 195. To a solution of P39.3 (0.406 g, 2.175 mmol) and chloropyrimidine (0.700 g, 2.175 mmol) in a vial in dioxane (3 ml) a TsOH-H₂O (0.413 g, 2.175 mmol) was added. The vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel column chromatography (EtOAc:n-hexane=1:1) to give Compound 195 (0.720 g, 71% yield) as a white solid. ¹H NMR (400 MHz, DMSO-d₆): δ 9.84 (br., 1H), 8.97-8.95 (m, 1H), 8.60 (s, 1H), 7.80-7.78 (m, 1H), 7.57-7.43 (m, 6H), 7.25-7.12 (m, 6H), 5.19-5.13 (m, 2H), 4.29 (q, J=7.0 Hz, 2H), 3.80-3.77 (m, 1H), 3.64-3.60 (m, 1H), 1.33 (t, J=7.0 Hz, 3H).

### Preparation 40: Ethyl 2-{[3-chloro-4-(trifluoromethyl)phenyl]amino}-4-{[(1S)-2-hydroxy-1-phenylethyl]amino}pyrimidine-5-carboxylate (Compound 248)

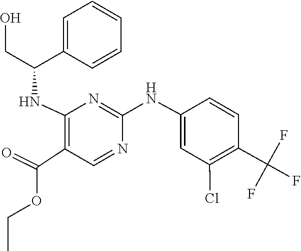 [SMILES: CCOC(=O)c1cnc(Nc2ccc(C(F)(F)F)c(Cl)c2)nc1N[C@H](CO)c1ccccc1]

The compound was synthesized according to the procedure described in [symbol] using 3-chloro-4-(trifluoromethyl)aniline instead of 4-sulfonylmethyl-3-methylaniline. ¹H NMR (400 MHz, DMSO-d₆): δ 10.26 (s, 1H), 9.06 (d, J=7.30 Hz, 1H), 8.66 (s, 1H), 7.99 (s, 1H), 7.70-7.62 (m, 2H), 7.40-7.20 (m, 5H), 5.25-5.22 (m, 2H), 4.31 (q, J=7.2 Hz, 2H), 3.88-3.69 (m, 2H), 1.34 (t, J=7.2 Hz, 3H).

### Preparation 41: Ethyl 4-{[(1S)-2-hydroxy-1-phenylethyl]amino}-2-[(2-methyl-1-oxo-1,2,3,4-tetrahydroisoquinolin-6-yl)amino]pyrimidine-5-carboxylate (Compound 122)

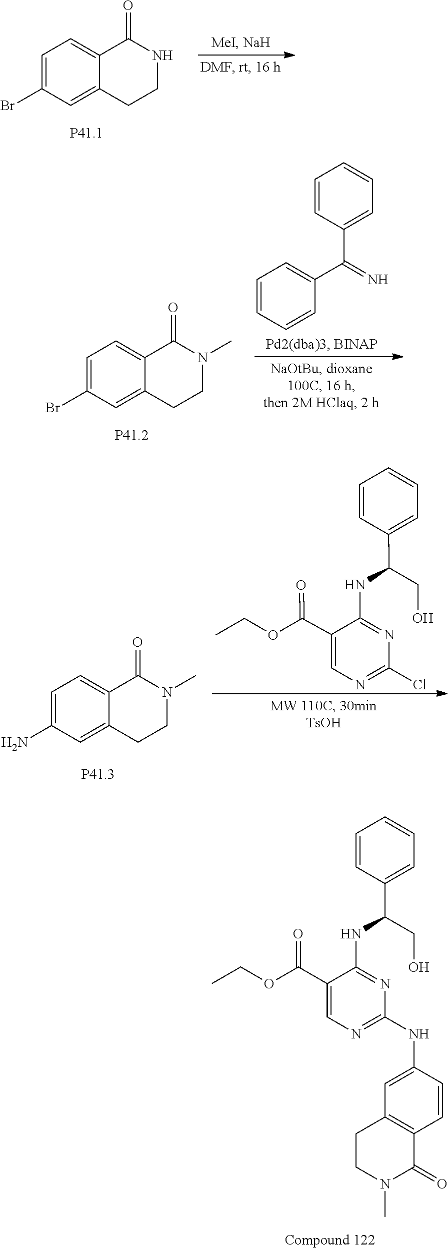

Synthesis of P41.2. To a mixture of 6-bromo-3,4-dihydroisoquinolin-1(2H)-one P41.1 (1.00 g, 4.42 mmol) in DMF (20 ml) a 60% NaH (0.230 g, 5.75 mmol) was added and mixture was stirred at rt for a 30 min. Then a Mel (0.82 g, 5.75 mmol) was added, and mixture was stirred at rt for a 16 h. A mixture was poured in cold 10% solution of citric acid (100 ml) and extracted with EtOAc (2×20 ml). An organic layer was washed with water, 10% solution of NaHCO₃, brine, dried under Na₂SO₄ and concentrated to give a residue. A residue was purified by silica gel column chromatography (EtOAc:n-hexane=1:1) to give P41.2 (0.65 g, 61% yield) as slight yellow solid. ¹H NMR (400 MHz, DMSO-d₆): 7.76 (d, J=8.1 Hz, 1H), 7.54-7.51 (m, 2H), 3.53 (t, J=6.7 Hz, 2H), 3.00-2.97 (m, 5H).

Synthesis of P41.3. To a solution of P41.2 (570.0 mg, 2.37 mmol) in dioxane (5 mL, degassed) 1,1-diphenylmethanimine (470.0 mg, 2.61 mmol), NaO^{t}Bu (460.0 mg, 4.74 mmol), BINAP (147.0 mg, 0.237 mmol), and Pd₂(dba)₃ (109.0 mg, 0.118 mmol) was added at room temperature under argon atmosphere. After the solution was stirred at 100° C. for overnight, the mixture was cooled and diluted with 10% HCl (10 ml) and stirred at rt for a 2 h. Then a solution was filtered through a pad of celite, and filtrate was then concentrated. A residue was dissolved in water (10 ml) and extracted with Et₂O (2×5 ml). An aqueous layer was neutralized with 10% solution of Na₂CO₃ to pH 8 and extracted with DCM (3×10 ml). Organic layer was washed with water, dried under Na₂SO₄, and concentrated to give a residue. A residue was recrystallized from Et₂O and give P41.3 (345 mg, 86% yield) as slight yellow solid. ¹H NMR (400 MHz, DMSO-d₆): 7.52 (d, J=8.6 Hz, 1H), 6.43 (d, J=6.4 Hz, 1H), 6.31 (s, 1H), 5.64 (br., 2H), 3.41 (t, J=6.6 Hz, 2H), 2.93 (s, 3H), 2.77 (t, J=6.5 Hz, 2H).

Synthesis of Compound 122. To a solution of P41.3 (200 mg, 1.13 mmol) and ethyl 2-chloro-4-{[(1R)-2-hydroxy-1-phenylethyl]amino}pyrimidine-5-carboxylate (365 mg, 1.13 mmol) in a vial in dioxane (3 ml) a TsOH-H₂O (215 mg, 1.13 mmol) was added. The vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel column chromatography (DCM:MeOH=50:1) to give Compound 122 (290 mg, 55% yield) as slight yellow solid. ¹H NMR (400 MHz, DMSO-d₆): 9.97 (s, 1H), 9.00 (d, J=7.2 Hz, 1H), 8.63 (s, 1H), 7.69 (d, J=8.3 Hz, 1H), 7.52-7.50 (m, 2H), 7.37-7.31 (m, 4H), 7.24-7.21 (m, 1H), 5.28-5.24 (m, 1H), 5.17-5.14 (m, 1H), 4.31 (q, J=7.0 Hz, 2H), 3.87-3.70 (m, 2H), 3.52-3.48 (m, 2H), 2.99 (s, 3H), 2.93-2.84 (m, 2H), 1.33 (t, J=7.0 Hz, 3H).

### Preparation 42: Ethyl 2-{[3-fluoro-4-(1-hydroxy-2-methylpropan-2-yl)phenyl]amino}-4-{[(1S)-2-hydroxy-1-phenylethyl]amino}pyrimidine-5-carboxylate

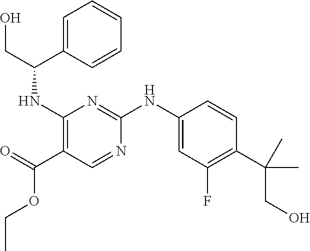 [SMILES: CCOC(=O)c1cnc(Nc2ccc(C(C)(C)CO)c(F)c2)nc1N[C@H](CO)c1ccccc1]

The compound was synthesized according to the procedure described in [symbol] using 2-(4-amino-2-fluorophenyl)-2-methylpropan-1-ol instead of 4-sulfonylmethyl-3-methylaniline. LCMS: [MH⁺] 469.

### Preparation 43: Ethyl 2-{[3-chloro-4-(1-hydroxy-2-methylpropan-2-yl)phenyl]amino}-4-{[(1S)-2-hydroxy-1-phenylethyl]amino}pyrimidine-5-carboxylate

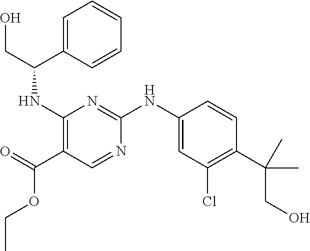 [SMILES: CCOC(=O)c1cnc(Nc2ccc(C(C)(C)CO)c(Cl)c2)nc1N[C@H](CO)c1ccccc1]

The compound was synthesized according to the procedure described in [symbol] using 2-(4-amino-2-chlorophenyl)-2-methylpropan-1-ol instead of 4-sulfonylmethyl-3-methylaniline. LCMS: [MH⁺] 485.

### Preparation 44: Ethyl 4-{[(1S)-2-hydroxy-1-phenylethyl]amino}-2-[(2-methyl-3-oxo-1,2,3,4-tetrahydroisoquinolin-7-yl)amino]pyrimidine-5-carboxylate—Compound 123

The compound was synthesized according to the procedure described in [symbol] using 7-amino-2-methyl-1,4-dihydroisoquinolin-3(2H)-one instead of 4-sulfonylmethyl-3-methylaniline.

### Preparation 45: Ethyl 2-{[4-(1-hydroxy-2-methylpropan-2-yl)phenyl]amino}-4-{[(1S)-2-hydroxy-1-phenylethyl]amino}pyrimidine-5-carboxylate—Compound 52

The compound was synthesized according to the procedure described in [symbol] using 2-(4-aminophenyl)-2-methylpropan-1-ol instead of 4-sulfonylmethyl-3-methylaniline.

### Preparation 46: Ethyl 2-{[2-chloro-4-(methylsulfonyl)phenyl]amino}-4-{[(1S)-2-hydroxy-1-phenylethyl]amino}pyrimidine-5-carboxylate—Compound 317

The compound was synthesized according to the procedure described in [symbol] using 2-chloro-4-(methylsulfonyl)aniline instead of 4-sulfonylmethyl-3-methylaniline.

### Preparation 47: Ethyl 2-{[2-fluoro-4-(methylsulfonyl)phenyl]amino}-4-{[(1S)-2-hydroxy-1-phenylethyl]amino}pyrimidine-5-carboxylate—Compound 211

The compound was synthesized according to the procedure described in [symbol] using 2-fluoro-4-(methylsulfonyl)aniline instead of 4-sulfonylmethyl-3-methylaniline.

### Preparation 48: Ethyl 4-{[(1S)-2-hydroxy-1-phenylethyl]amino}-2-{[2-methoxy-4-(methylsulfonyl)phenyl]amino}pyrimidine-5-carboxylate—Compound 299

The compound was synthesized according to the procedure described in [symbol] using 2-methoxy-4-(methylsulfonyl)aniline instead of 4-sulfonylmethyl-3-methylaniline.

### Preparation 49: Ethyl 4-{[(1S)-2-hydroxy-1-phenylethyl]amino}-2-{[2-methyl-4-(methylsulfonyl)phenyl]amino}pyrimidine-5-carboxylate—Compound 167

The compound was synthesized according to the procedure described in [symbol] using 2-methyl-4-(methylsulfonyl)aniline instead of 4-sulfonylmethyl-3-methylaniline.

### Preparation 50: Ethyl 2-{[3-chloro-4-(methylsulfonyl)phenyl]amino}-4-{[(1S)-2-hydroxy-1-phenylethyl]amino}pyrimidine-5-carboxylate—Compound 318

The compound was synthesized according to the procedure described in [symbol] using 3-chloro-4-(methylsulfonyl)aniline instead of 4-sulfonylmethyl-3-methylaniline.

### Preparation 51: 4-[[5-ethoxycarbonyl-4-[[(1S)-2-hydroxy-1-phenyl-ethyl]amino]pyrimidin-2-yl]amino]-2-methoxy-benzoic Acid—Compound 62

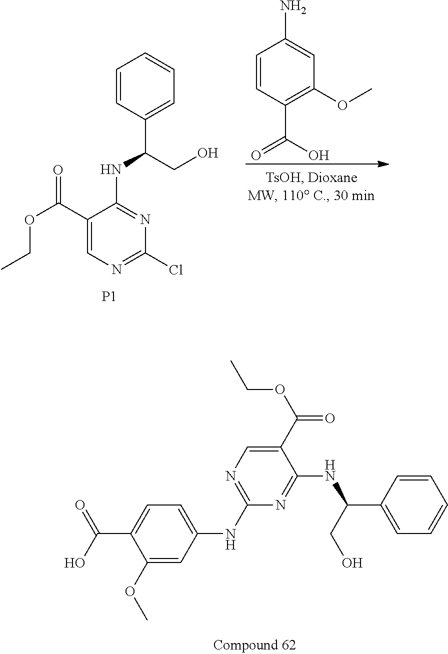

Compound 62 was synthesized according to the procedure described in [symbol] using P1 (100 mg, 0.31 mmol) and 4-amino-2-methoxybenzoic acid. Yield 80 mg, 57%. ¹H-NMR (400 MHz, dmso-d₆) δ: 12.12 (br. S, 1H), 9.94 (s, 1H), 8.98 (d, J=7.8 Hz, 1H), 8.64 (s, 1H), 7.61 (d, J=9.0 Hz, 1H), 7.44-7.29 (m, 6H), 7.27-7.17 (m, 1H), 5.39-5.26 (m, 1H), 5.22-5.05 (m, 1H), 4.31 (q, J=6.9 Hz, 2H), 3.92-3.81 (m, 1H), 3.79-3.67 (m, 4H), 1.34 (t, J=6.8 Hz, 3H).

### Preparation 52: Ethyl 4-{[(1S)-2-hydroxy-1-phenylethyl]amino}-2-{[4-(methylsulfonyl)phenyl]amino}pyrimidine-5-carboxylate—Compound 68

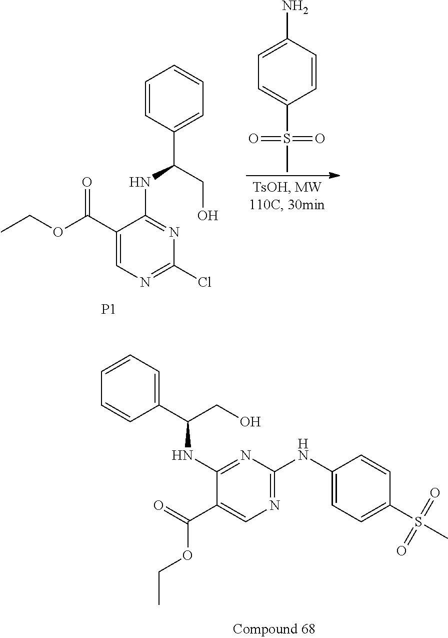

The compound was synthesized according to the procedure described in [symbol] using 4-(methylsulfonyl)aniline instead of 4-sulfonylmethyl-3-methylaniline. ¹H NMR (400 MHz, DMSO-d₆): 10.21 (s, 1H), 9.02 (d, J=6.8 Hz, 1H), 8.65 (s, 1H), 7.70-7.71 (m, 4H), 7.41-7.33 (m, 4H), 7.26-7.22 (m, 1H), 5.25-5.05 (m, 2H), 4.32 (q, J=7.0 Hz, 2H), 3.86-3.68 (m, 2H), 3.15 (s, 3H), 1.34 (t, J=7.0 Hz, 3H).

### Preparation 53: Ethyl 2-{[4-(ethylsulfonyl)phenyl]amino}-4-{[(1S)-2-hydroxy-1-phenylethyl]amino}pyrimidine-5-carboxylate—Compound 168

The compound was synthesized according to the procedure described in [symbol] using 4-(ethylsulfonyl)aniline instead of 4-sulfonylmethyl-3-methylaniline.

### Preparation 54: Ethyl 4-{[(1S)-2-hydroxy-1-phenylethyl]amino}-2-{[4-(propan-2-ylsulfonyl)phenyl]amino}pyrimidine-5-carboxylate—Compound 274

The compound was synthesized according to the procedure described in [symbol] using 4-[(1-methylethyl)sulfonyl]aniline instead of 4-sulfonylmethyl-3-methylaniline.

### Preparation 55: Ethyl 2-[(3,3-dioxido-1,3-benzoxathiol-5-yl)amino]-4-{[(1S)-2-hydroxy-1-phenylethyl]amino}pyrimidine-5-carboxylate—Compound 164

The compound was synthesized according to the procedure described in [symbol] using 1,3-benzoxathiol-5-amine 3,3-dioxide instead of 4-sulfonylmethyl-3-methylaniline.

### Preparation 56: Ethyl 2-[(2-ethyl-1-oxo-3,4-dihydroisoquinolin-6-yl)amino]-4-[[(1S)-2-hydroxy-1-phenyl-ethyl]amino]pyrimidine-5-carboxylate—Compound 218

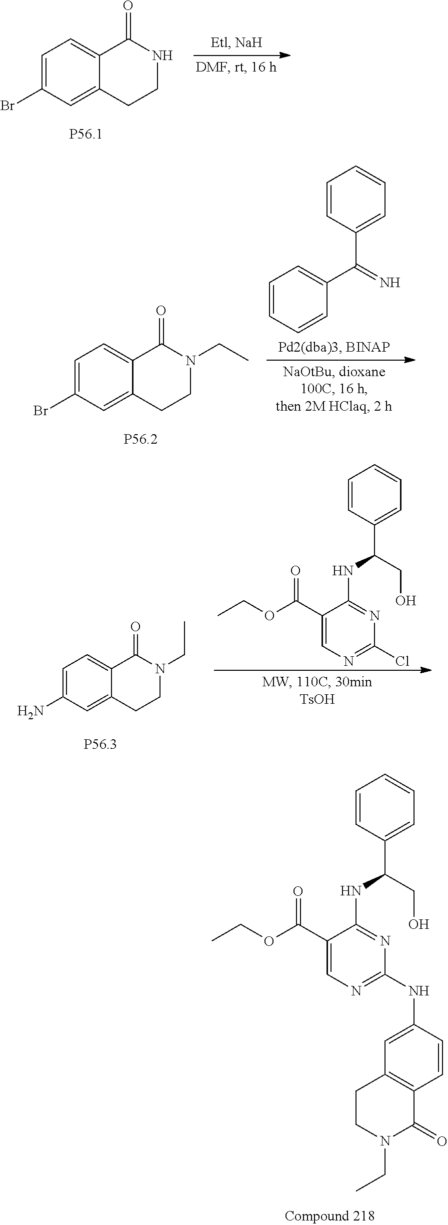

Synthesis of P56.2. To a mixture of 6-bromo-3,4-dihydroisoquinolin-1(2H)-one P56.1 (0.500 g, 1.88 mmol) in DMF (20 ml) a 60% NaH (0.112 g, 2.82 mmol) was added and mixture was stirred at rt for a 30 min. Then a EtI (0.44 g, 2.82 mmol) was added, and mixture was stirred at rt for a 16 h. A mixture was poured in cold 10% solution of citric acid (100 ml) and extracted with EtOAc (2×20 ml). An organic layer was washed with water, 10% solution of NaHCO₃, brine, dried under Na₂SO₄ and concentrated to give a residue. A residue was purified by silica gel column chromatography (EtOAc:n-hexane=1:1) to give P56.2 (0.48 g, 85% yield). ¹H NMR (400 MHz, DMSO-d₆): δ 7.76 (d, J=8.1 Hz, 1H), 7.54-7.51 (m, 2H), 3.54-3.46 (m, 4H), 2.96 (t, J=6.5 Hz, 2H), 1.10 (t, J=7.2 Hz, 3H).

Synthesis of P56.3. To a solution of P56.2 (480.0 mg, 1.89 mmol) in dioxane (5 mL, degassed) 1,1-diphenylmethanimine (376.0 mg, 2.08 mmol), NaO^{t}Bu (362.0 mg, 3.78 mmol), BINAP (125.0 mg, 0.189 mmol), and Pd₂(dba)₃ (86.0 mg, 0.098 mmol) was added at room temperature under argon atmosphere. After the solution was stirred at 100° C. for overnight, the mixture was cooled and diluted with 10% HCl (10 ml) and stirred at rt for a 2 h. Then a solution was filtered through a pad of celite, and filtrate was then concentrated. A residue was dissolved in water (10 ml) and extracted with Et₂O (2×5 ml). An aqueous layer was neutralized with 10% solution of Na₂CO₃ to pH 8 and extracted with DCM (3×10 ml). Organic layer was washed with water, dried under Na₂SO₄ and concentrated to give a residue. A residue was recrystallized from Et₂O and give P56.3 (150 mg, 42% yield). ¹H NMR (400 MHz, DMSO-d₆): δ 7.52 (d, J=8.3 Hz, 1H), 6.43 (dd, J1=8.4 Hz, J2=2.3 Hz, 1H), 6.31 (s, 1H), 5.62 (br, 2H), 3.45-3.38 (m, 4H), 2.75 (t, J=6.6 Hz, 2H), 1.05 (t, J=7.1 Hz, 3H).

Synthesis of Compound 218. To a solution of P56.3 (150 mg, 0.79 mmol) and ethyl 2-chloro-4-{[(1S)-2-hydroxy-1-phenylethyl]amino}pyrimidine-5-carboxylate ( [symbol] , 254 mg, 0.79 mmol) in a vial in dioxane (3 ml) a TsOH-H₂O (150 mg, 0.79 mmol) was added. A vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel column chromatography (DCM:MeOH=50:1) to give Compound 218 (135 mg, 36% yield). ¹H NMR (400 MHz, DMSO-d₆): δ 9.96 (s, 1H), 8.98 (d, J=7.3 Hz, 1H), 8.62 (s, 1H), 7.69 (d, J=8.4 Hz, 1H), 7.52-7.49 (m, 2H), 7.38-7.31 (m, 4H), 7.25-7.21 (m, 1H), 5.29-5.25 (m, 1H), 5.14 (t, J=4.6 Hz, 1H), 4.31 (q, J=7.1 Hz, 2H), 3.87-3.81 (m, 1H), 3.75-3.71 (m, 1H), 3.52-3.41 (m, 4H), 2.94-2.82 (m, 2H), 1.33 (t, J=7.1 Hz, 3H), 1.10 (t, J=7.0 Hz, 3H).

### Preparation 57: Ethyl 4-[[(1S)-2-hydroxy-1-phenyl-ethyl]amino]-2-[(2-isopropyl-1-oxo-3,4-dihydroisoquinolin-6-yl)amino]pyrimidine-5-carboxylate—Compound 316

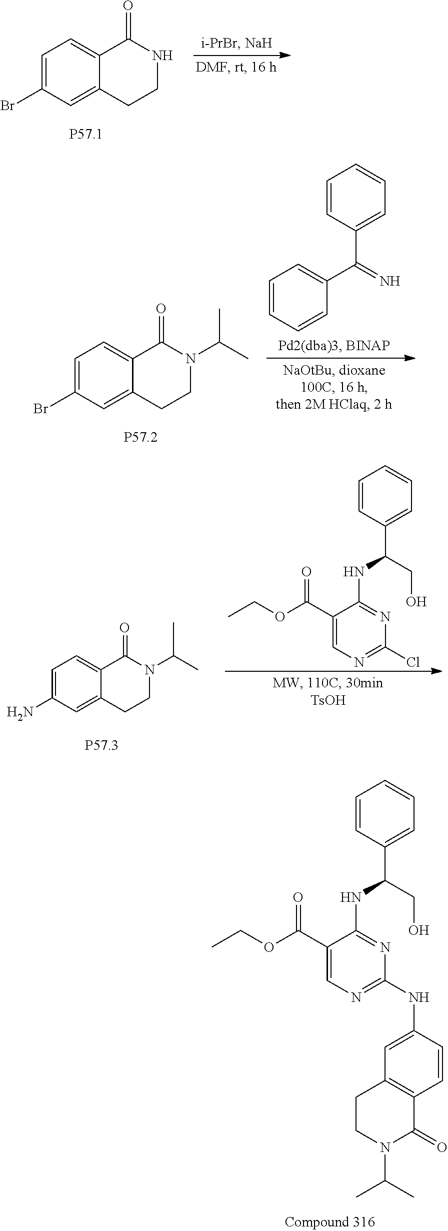 [SMILES: O=C1NCCc2cc(Br)ccc21 ; CC(C)N1CCc2cc(Br)ccc2C1=O ; N=C(c1ccccc1)c1ccccc1 ; CC(C)N1CCc2cc(N)ccc2C1=O ; CCOC(=O)c1cnc(Cl)nc1N[C@H](CO)c1ccccc1 ; CCOC(=O)c1cnc(Nc2ccc3c(c2)CCN(C(C)C)C3=O)nc1N[C@H](CO)c1ccccc1]

Synthesis of P57.2. To a solution of 6-bromo-3,4-dihydroisoquinolin-1(2H)-one P57.1 (0.500 g, 1.88 mmol) in DMF (20 ml) a 60% NaH (0.112 g, 2.82 mmol) was added and mixture was stirred at rt for a 30 min. Then a i-PrBr (0.347 g, 2.82 mmol) was added, and mixture was stirred at rt for a 16 h. A mixture was poured in cold 10% solution of citric acid (100 ml) and extracted with EtOAc (2×20 ml). An organic layer was washed with water, 10% solution of NaHCO₃, brine, dried under Na₂SO₄ and concentrated to give a residue. A residue was purified by silica gel column chromatography (EtOAc:n-hexane=1:1) to give P57.2 (0.34 g, 72% yield). ¹H NMR (400 MHz, DMSO-d₆): δ 7.77 (d, J=7.9 Hz, 1H), 7.54-7.51 (m, 2H), 4.86-4.80 (m, 1H), 3.41 (t, J=6.7 Hz, 2H), 2.92 (t, J=6.2 Hz, 2H), 1.12 (d, J=6.7 Hz, 6H).

Synthesis of P57.3. To a solution of P57.2 (340 mg, 1.89 mmol) in dioxane (5 mL, degassed) 1,1-diphenylmethanimine (275 mg, 1.52 mmol), NaO^{t}Bu (243 mg, 3.78 mmol), BINAP (58 mg, 0.127 mmol), and Pd₂(dba)₃ (58 mg, 0.063 mmol) was added at room temperature under argon atmosphere. After the solution was stirred at 100° C. for overnight, the mixture was cooled and diluted with 10% HCl (10 ml) and stirred at rt for a 2 h. Then a solution was filtered through a pad of celite, and filtrate was then concentrated. A residue was dissolved in water (10 ml) and extracted with Et₂O (2×5 ml). An aqueous layer was neutralized with 10% solution of Na₂CO₃ to pH 8 and extracted with DCM (3×10 ml). Organic layer was washed with water, dried under Na₂SO₄ and concentrated to give a residue. A residue was recrystallized from Et₂O and give P57.3 (145 mg, 56% yield). ¹H NMR (400 MHz, DMSO-d₆): δ 7.53 (d, J=8.4 Hz, 1H), 6.44 (dd, J1=8.6 Hz, J2=2.1 Hz, 1H), 6.32 (s, 1H), 5.61 (br, 2H), 4.84-4.77 (m, 1H), 3.38 (t, J=6.6 Hz, 2H), 2.71 (t, J=6.4 Hz, 2H), 1.07 (d, J=6.6 Hz, 6H).

Synthesis of Compound 316. To a solution of P57.3 (145 mg, 0.71 mmol) and ethyl 2-chloro-4-{[(1S)-2-hydroxy-1-phenylethyl]amino}pyrimidine-5-carboxylate (228 mg, 0.71 mmol) in a vial in dioxane (3 ml) a TsOH-H₂O (135 mg, 0.71 mmol) was added. A vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel column chromatography (DCM:MeOH=50:1) to give Compound 316 (145 mg, 40% yield). ¹H NMR (400 MHz, DMSO-d₆): δ 9.55 (s, 1H), 8.98 (d, J-7.2 Hz, 1H), 8.62 (s, 1H), 7.69 (d, J=8.4 Hz, 1H), 7.52-7.50 (m, 2H), 7.38-7.32 (m, 4H), 7.25-7.21 (m, 1H), 5.29-5.25 (m, 1H), 5.15 (t, J=4.8 Hz, 1H), 4.88-4.80 (m, 1H), 4.31 (q, J=7.2 Hz, 2H), 3.87-3.82 (m, 1H), 3.75-3.70 (m, 1H), 3.38 (t, J=6.6 Hz, 2H), 2.88-2.78 (m, 2H), 1.33 (t, J=7.1 Hz, 3H), 1.13 (d, J=6.7 Hz, 6H).

### Preparation of Carbonic Acids.

### Preparation 58: 4-{[(1S)-2-hydroxy-1-phenylethyl]amino}-2-{[3-methyl-4-(methylsulfonyl)phenyl]amino}pyrimidine-5-carboxylic Acid

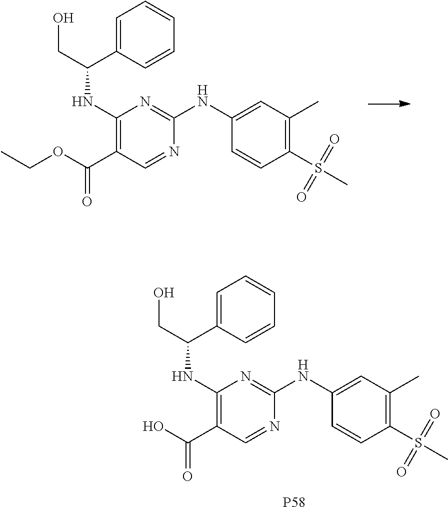

A solution of KOH (0.223 g, 3.98 mmol) in H₂O (1 mL) was added to a solution of ethyl 4-{[(1S)-2-hydroxy-1-phenylethyl]amino}-2-{[3-methyl-4-(methylsulfonyl)phenyl]-amino}pyrimidine-5-carboxylate (0.75 g, 1.55 mmol) in EtOH (5 mL). The mixture was stirred at ambient temperature for a 16 h, concentrated under reduced pressure, diluted with H₂O (3 mL), and acidified with 10% solution of citric acid. Formed precipitate was filtered off, washed with H₂O, and dried at 50° C. to afford 0.48 g (70%) of the title compound as a white solid. ¹H NMR (400 MHz, DMSO-d₆): δ 12.86 (br, 1H), 10.00 (s, 1H), 9.14 (d, J=7.9 Hz, 1H), 8.60 (s, 1H), 7.73-7.66 (m, 2H), 7.57 (s, 1H), 7.39-7.22 (m, 5H), 5.29-5.11 (m, 2H), 3.86-3.70 (m, 2H), 3.14 (s, 3H), 2.54 (s, 3H).

### Preparation 59: 2-{[3-fluoro-4-(methylsulfonyl)phenyl]amino}-4-{[(1S)-2-hydroxy-1-phenylethyl]amino}pyrimidine-5-carboxylic Acid—Compound 42

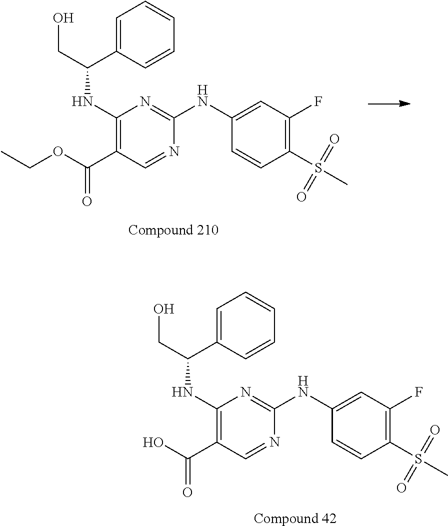 [SMILES: CCOC(=O)c1cnc(Nc2ccc(S(C)(=O)=O)c(F)c2)nc1N[C@H](CO)c1ccccc1 ; CS(=O)(=O)c1ccc(Nc2ncc(C(=O)O)c(N[C@H](CO)c3ccccc3)n2)cc1F ; "Compound 42"]

1N NaOH (7 ml, 0.007 mol) was added to solution of Compound 210 ((1.4 g, 0.0029 mol) in EtOH (10 ml) and the reaction mixture was stirred at rt for overnight. EtOH was evaporated; the residue was acidified with diluted HCl. The formed solid was collected by filtration, washed with water, dry ether to give pure acid—Compound 42. The yield is 1.0 g, 77%. ¹H NMR (400 MHz, DMSO-d₆): δ 12.96 (br, 1H), 10.39 (s, 1H), 9.20 (d, J=8.2 Hz, 1H), 8.67 (s, 1H), 7.78 (d, J=13.3 Hz, 1H), 7.65 (t, J=8.3 Hz, 1H), 7.49 (d, J=8.3 Hz, 1H), 7.39-7.21 (m, 5H), 5.23-5.19 (m, 2H), 3.88-3.68 (m, 2H), 3.25 (s, 3H).

### Preparation 60: 4-{[(1S)-2-hydroxy-1-phenylethyl]amino}-2-[(2-methyl-1-oxo-1,2,3,4-tetrahydroisoquinolin-6-yl)amino]pyrimidine-5-carboxylic Acid

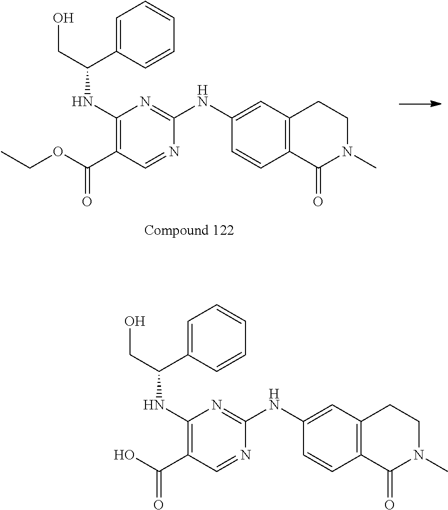 [SMILES: CCOC(=O)c1cnc(Nc2ccc3c(c2)CCN(C)C3=O)nc1N[C@H](CO)c1ccccc1 ; CN1CCc2cc(Nc3ncc(C(=O)O)c(N[C@H](CO)c4ccccc4)n3)ccc2C1=O]

To a solution of the Compound 122 (250 mg, 0.54 mmol) in EtOH (2 ml) a solution of KOH (75 mg, 1.35 mmol) in H₂O (0.5 ml) was added and mixture was stirred at rt for a 16 h. Then a mixture was concentrated, diluted with H₂O (3 ml) and acidified with 10% solution of citric acid. A solid was filtered, washed with H₂O, and dried at 50° C. to give the title compound (175 mg, 75% yield) as white solid. ¹H NMR (400 MHz, DMSO-d₆): 12.71 (br., 1H), 9.84 (s, 1H), 9.11-9.10 (m, 1H), 8.58 (s, 1H), 7.70 (d, J=Hz, 1H), 7.54-7.51 (m, 2H), 7.37-7.22 (m, 5H), 5.29-5.25 (m, 1H), 5.12-5.09 (m, 1H), 3.85-3.70 (m, 2H), 3.53-3.48 (m, 2H), 3.01-2.84 (m, 5H).

### Preparation 61: 2-[(6-fluorobiphenyl-3-yl)amino]-4-{[(1S)-2-hydroxy-1-phenylethyl]amino}pyrimidine-5-carboxylic Acid

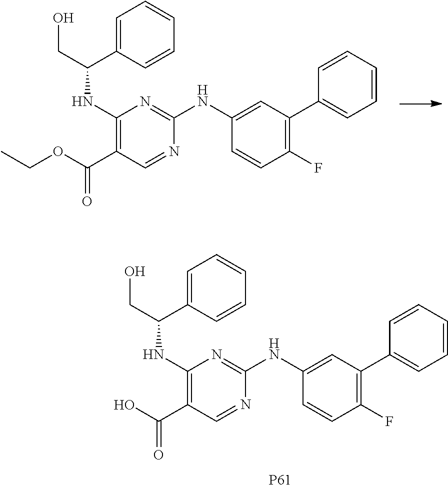

The compound was synthesized according to the procedure described in [symbol] using ethyl 2-[(6-fluorobiphenyl-3-yl)amino]-4-{[(1S)-2-hydroxy-1-phenylethyl]amino}pyrimidine-5-carboxylate of ethyl 4-{[(1S)-2-hydroxy-1-phenylethyl]amino}-2-{[3-methyl-4-(methylsulfonyl)phenyl]amino}pyrimidine-5-carboxylate. ¹H NMR (400 MHz, DMSO-d₆): δ 12.70 (br., 1H), 9.76 (br., 1H), 9.11-9.09 (m, 1H), 8.56 (s, 1H), 7.80-7.77 (m, 1H), 7.57-7.43 (m, 6H), 7.25-7.12 (m, 6H), 5.20-5.13 (m, 2H), 3.79-3.75 (m, 1H), 3.62-3.59 (m, 1H).

### Preparation 62: 2-{[3-chloro-4-(trifluoromethyl)phenyl]amino}-4-{[(1S)-2-hydroxy-1-phenylethyl]amino}pyrimidine-5-carboxylic Acid

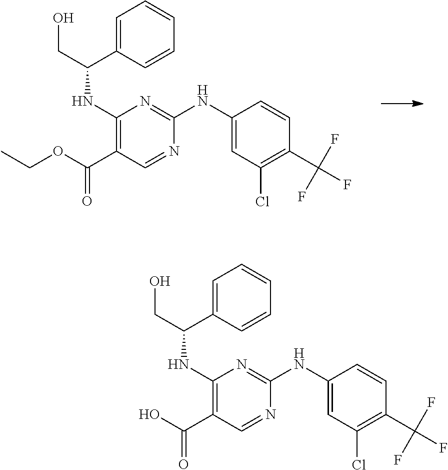 [SMILES: CCOC(=O)c1cnc(Nc2ccc(C(F)(F)F)c(Cl)c2)nc1N[C@H](CO)c1ccccc1 ; O=C(O)c1cnc(Nc2ccc(C(F)(F)F)c(Cl)c2)nc1N[C@H](CO)c1ccccc1]

The compound was synthesized according to the procedure described in [symbol] using ethyl 2-{[3-chloro-4-(trifluoromethyl)phenyl]amino}-4-{[(1S)-2-hydroxy-1-phenylethyl]amino}pyrimidine-5-carboxylate instead of ethyl 4-{[(1S)-2-hydroxy-1-phenylethyl]amino}-2-{[3-methyl-4-(methylsulfonyl)phenyl]amino}-pyrimidine-5-carboxylate. ¹H NMR (400 MHz, DMSO-d₆): δ 12.98 (br., 1H), 10.17 (s, 1H), 9.19 (d, J=7.2 Hz, 1H), 8.62 (s, 1H), 8.00 (s, 1H), 7.70-7.62 (m, 2H), 7.39-7.20 (m, 5H), 5.27-5.22 (m, 2H), 3.86-3.68 (m, 2H).

### Preparation 63: 4-{[(1S)-2-hydroxy-1-phenylethyl]amino}-2-[(2-methyl-3-oxo-1,2,3,4-tetrahydroisoquinolin-7-yl)amino]pyrimidine-5-carboxylic Acid

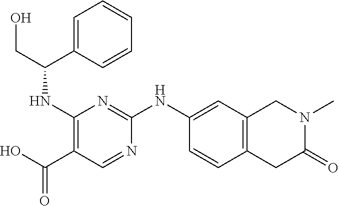 [SMILES: CN1Cc2cc(Nc3ncc(C(=O)O)c(N[C@H](CO)c4ccccc4)n3)ccc2CC1=O]

The compound was synthesized according to the procedure described in [symbol] using ethyl 4-{[(1S)-2-hydroxy-1-phenylethyl]amino}-2-[(2-methyl-3-oxo-1,2,3,4-tetrahydroisoquinolin-7-yl)amino]pyrimidine-5-carboxylate instead of ethyl 4-{[(1S)-2-hydroxy-1-phenylethyl]amino}-2-{[3-methyl-4-(methylsulfonyl)phenyl]amino}-pyrimidine-5-carboxylate. LCMS: [MH⁺] 434.

### Preparation 64: 2-{[4-(2-hydroxy-1,1-dimethylethyl)phenyl]amino}-4-{[(1S)-2-hydroxy-1-phenylethyl]amino}pyrimidine-5-carboxylic Acid

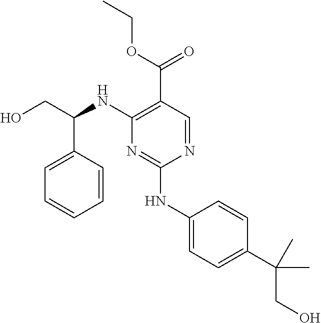 [SMILES: CCOC(=O)c1cnc(Nc2ccc(C(C)(C)CO)cc2)nc1N[C@H](CO)c1ccccc1]

The compound was synthesized according to the procedure described in [symbol] using ethyl 2-{[4-(2-hydroxy-1,1-dimethylethyl)phenyl]amino}-4-{[(1S)-2-hydroxy-1-phenylethyl]amino}pyrimidine-5-carboxylate instead of ethyl 4-{[(1S)-2-hydroxy-1-phenylethyl]amino}-2-{[3-methyl-4-(methylsulfonyl)phenyl]amino}pyrimidine-5-carboxylate. LCMS: [MH⁺] 451.

### Preparation 65: 2-{[3-chloro-4-(2-hydroxy-1,1-dimethylethyl)phenyl]amino}-4-{[(1S)-2-hydroxy-1-phenylethyl]amino}pyrimidine-5-carboxylic Acid

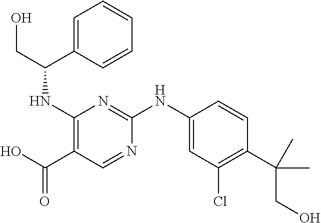 [SMILES: CC(C)(CO)c1ccc(Nc2ncc(C(=O)O)c(N[C@H](CO)c3ccccc3)n2)cc1Cl]

The compound was synthesized according to the procedure described in [symbol] using ethyl 2-{[3-chloro-4-(2-hydroxy-1,1-dimethylethyl)phenyl]amino}-4-{[(1S)-2-hydroxy-1-phenylethyl]amino}pyrimidine-5-carboxylate instead of ethyl 4-{[(1S)-2-hydroxy-1-phenylethyl]amino}-2-{[3-methyl-4-(methylsulfonyl)phenyl]amino}-pyrimidine-5-carboxylate. LCMS: [MH⁺] 457.

### Representative Examples of the Compound

### Example 1: Ethyl 2-{[3-fluoro-4-(methylsulfonyl)phenyl]amino}-4-{[(1S)-2-hydroxy-1-phenylethyl]amino}pyrimidine-5-carboxylate—Compound 210

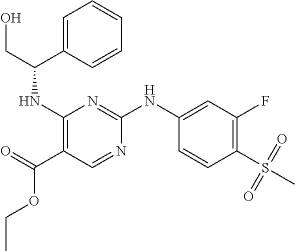 [SMILES: CCOC(=O)c1cnc(Nc2ccc(S(C)(=O)=O)c(F)c2)nc1N[C@H](CO)c1ccccc1]

The compound was synthesized according to the procedure described in [symbol] . ¹H NMR (400 MHz, DMSO-d₆): δ 10.39 (s, 1H), δ 9.06 (d, J=8.2 Hz, 1H), 8.67 (s, 1H), 7.78 (d, J=13.3 Hz, 1H), 7.65 (t, J=8.3 Hz, 1H), 7.49 (d, J=8.3 Hz, 1H), 7.39-7.21 (m, 5H), 5.23-5.19 (m, 2H), 4.33 (q, J=7.0 Hz, 2H), 3.88-3.68 (m, 2H), 3.25 (s, 3H), 1.34 (t, J=7.0 Hz, 3H).

### Example 2: Ethyl 4-{[(1S)-2-hydroxy-1-phenylethyl]amino}-2-[(1-oxo-1,2,3,4-tetrahydroisoquinolin-6-yl)amino]pyrimidine-5-carboxylate—Compound 45

The compound was synthesized according to the procedure described in [symbol] .

### Example 3 Ethyl 2-[(6-fluorobiphenyl-3-yl)amino]-4-{[(1S)-2-hydroxy-1-phenylethyl]amino}pyrimidine-5-carboxylate—Compound 195

The compound was synthesized according to the procedure described in [symbol] .

### Example 4: Ethyl 2-{[3-chloro-4-(trifluoromethyl)phenyl]amino}-4-{[(1S)-2-hydroxy-1-phenylethyl]amino}pyrimidine-5-carboxylate—Compound 248

The compound was synthesized according to the procedure described in [symbol] .

### Example 5: Ethyl 4-{[(1S)-2-hydroxy-1-phenylethyl]amino}-2-[(2-methyl-1-oxo-1,2,3,4-tetrahydroisoquinolin-6-yl)amino]pyrimidine-5-carboxylate—Compound 122

The compound was synthesized according to the procedure described in [symbol] .

### Example 6: Ethyl 2-{[3-fluoro-4-(1-hydroxy-2-methylpropan-2-yl)phenyl]-amino}-4-{[(1S)-2-hydroxy-1-phenylethyl]amino}pyrimidine-5-carboxylate—Compound 152

The compound was synthesized according to the procedure described in [symbol] .

### Example 7: Ethyl 2-{[3-chloro-4-(1-hydroxy-2-methylpropan-2-yl)phenyl]amino}-4-{[(1S)-2-hydroxy-1-phenylethyl]amino}pyrimidine-5-carboxylate—Compound 275

The compound was synthesized according to the procedure described in [symbol] .

### Example 8 Ethyl 4-{[(1S)-2-hydroxy-1-phenylethyl]amino}-2-{[3-methyl-4-(methylsulfonyl)phenyl]amino}pyrimidine-5-carboxylate—Compound 166

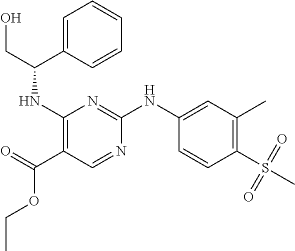 [SMILES: CCOC(=O)c1cnc(Nc2ccc(S(C)(=O)=O)c(C)c2)nc1N[C@H](CO)c1ccccc1]

The compound was synthesized according to the procedure described in [symbol] . ¹H NMR (400 MHz, DMSO-d₆): δ 10.12 (s, 1H), 9.01 (d, J=8.2 Hz, 1H), 8.64 (s, 1H), 7.72-7.64 (m, 2H), 7.38-7.21 (m, 5H), 5.23-5.19 (m, 2H), 4.33 (q, J=7.0 Hz, 2H), 3.88-3.68 (m, 2H), 3.14 (s, 3H), 2.54 (s, 3H), 1.34 (t, J=7.0 Hz, 3H).

### Example 9: Ethyl 2-{[2-fluoro-4-(methylsulfonyl)phenyl]amino}-4-{[(1S)-2-hydroxy-1-phenylethyl]amino}pyrimidine-5-carboxylate—Compound 211

The compound was synthesized according to the procedure described in [symbol] . ¹H-NMR (400 MHz, dmso-d6) δ: 9.68 (br s, 1H), 9.01 (d, J=7.5 Hz, 1H), 8.64 (s, 1H), 7.89 (t, J=7.5 Hz, 1H), 7.78-7.72 (m, 1H), 7.62-7.55 (m, 1H), 7.35-7.27 (m, 4H), 7.26-7.20 (m, 1H), 5.16-5.09 (m, 1H), 4.45-4.41 (m, 1H), 4.32 (q, J=7.2 Hz, 2H), 3.83-3.76 (m, 1H), 3.71-3.64 (m, 1H), 3.25 (s, 3H), 1.34 (t, J=6.7 Hz, 3H).

### Example 10: Ethyl 4-{[(1S)-2-hydroxy-1-phenylethyl]amino}-2-{[2-methoxy-4-(methylsulfonyl)phenyl]amino}pyrimidine-5-carboxylate—Compound 299

The compound was synthesized according to the procedure described in [symbol] . ¹H-NMR (400 MHz, dmso-d₆) δ: 9.02 (d, J=7.0 Hz, 1H), 8.63 (s, 1H), 8.29 (s, 1H), 8.10 (d, J=8.8 Hz, 1H), 7.47-7.30 (m, 6H), 7.28-7.20 (m, 1H), 5.26-5.12 (m, 2H), 4.32 (q, J=6.7 Hz, 2H), 3.93 (s, 3H), 3.86-3.78 (m, 1H), 3.74-3.65 (m, 1H), 3.20 (s, 3H), 1.34 (t, J=7.4 Hz, 3H).

### Example 11: Ethyl 4-{[(1S)-2-hydroxy-1-phenylethyl]amino}-2-{[2-methyl-4-(methylsulfonyl)phenyl]amino}pyrimidine-5-carboxylate—Compound 167

The compound was synthesized according to the procedure described in [symbol] . ¹H-NMR (400 MHz, dmso-d₆) δ: 9.09 (br s, 1H), 8.92-8.87 (m, 1H), 8.60 (s, 1H), 7.70 (s, 1H), 7.55 (s, 2H), 7.34-7.28 (m, 2H), 7.26-7.18 (m, 3H), 5.14-5.08 (m, 1H), 5.07-4.98 (m, 1H), 4.30 (q, J=7.1 Hz, 2H), 3.79-3.69 (m, 1H), 3.68-3.59 (m, 1H), 3.19 (s, 3H), 3.23 (s, 3H), 1.33 (t, J=6.8 Hz, 3H).

### Example 12: Ethyl 2-{[3-chloro-4-(methylsulfonyl)phenyl]amino}-4-{[(1S)-2-hydroxy-1-phenylethyl]amino}pyrimidine-5-carboxylate—Compound 318

The compound was synthesized according to the procedure described in [symbol] .

### Example 13: Ethyl 4-{[(1S)-2-hydroxy-1-phenylethyl]amino}-2-{[3-methoxy-4-(methylsulfonyl)phenyl]amino}pyrimidine-5-carboxylate—Compound 300

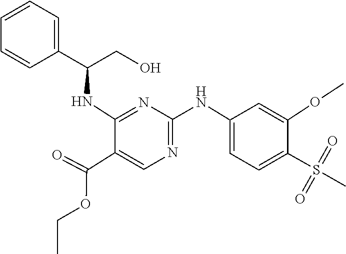 [SMILES: CCOC(=O)c1cnc(Nc2ccc(S(C)(=O)=O)c(OC)c2)nc1N[C@H](CO)c1ccccc1]

The compound was synthesized according to the procedure described in [symbol] using appropriate aniline. ¹H-NMR (400 MHz, dmso-d6) δ: 10.09 (br s, 1H), 8.99 (d, J=7.4 Hz, 1H), 8.66 (s, 1H), 7.60 (d, J=8.7 Hz, 1H), 7.51-7.42 (m, 2H), 7.40-7.30 (m, 4H), 7.28-7.21 (m, 1H), 5.35-5.25 (m, 1H), 5.16 (t, J=8.0 Hz, 1H), 4.31 (q, J=6.9 Hz, 2H), 3.89-3.80 (m, 4H), 3.79-3.71 (m, 1H), 3.17 (s, 3H), 1.34 (t, J=7.0 Hz, 3H).

### Example 14: Ethyl 4-{[(1S)-2-hydroxy-1-phenylethyl]amino}-2-{[4-(methylsulfonyl)-phenyl]amino}pyrimidine-5-carboxylate—Compound 68

The compound was synthesized according to the procedure described in [symbol] .

### Example 15: Ethyl 2-{[4-(ethylsulfonyl)phenyl]amino}-4-{[(1S)-2-hydroxy-1-phenylethyl]amino}pyrimidine-5-carboxylate—Compound 168

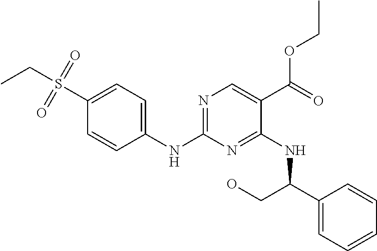

The compound was synthesized according to the procedure described in [symbol] . ¹H-NMR (400 MHz, dmso-d₆) δ: 10.21 (br s, 1H), 9.02 (d, J=7.4 Hz, 1H), 8.65 (s, 1H), 7.77 (d, J=8.7 Hz, 2H), 7.68 (d, J=8.7 Hz, 2H), 7.39 (d, J=7.5 Hz, 2H), 7.34 (t, J=7.7 Hz, 2H), 7.23 (t, J=7.2 Hz, 1H), 5.26-5.16 (m, 2H), 4.32 (q, J=7.2 Hz, 2H), 3.89-3.80 (m, 1H), 3.75-3.65 (m, 1H), 3.21 (q, J=7.8 Hz, 2H), 1.34 (t, J=7.0 Hz, 3H), 1.10 (t, J=7.4 Hz, 3H).

### Example 16: Ethyl 4-{[(1S)-2-hydroxy-1-phenylethyl]amino}-2-{[4-(propan-2-ylsulfonyl)phenyl]amino}pyrimidine-5-carboxylate—Compound 274

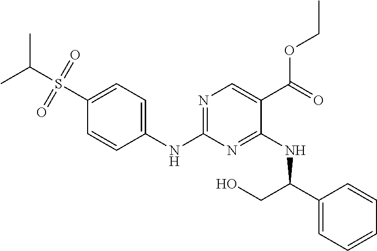 [SMILES: CCOC(=O)c1cnc(Nc2ccc(S(=O)(=O)C(C)C)cc2)nc1N[C@H](CO)c1ccccc1]

The compound was synthesized according to the procedure described in [symbol] . ¹H-NMR (400 MHz, dmso-d₆) δ: 10.23 (br. s, 1H), 9.03 (d, J=6.9 Hz, 1H), 8.65 (s, 1H), 7.78 (d, J=8.7 Hz, 2H), 7.64 (d, J=8.5 Hz, 2H), 7.39 (d, J=7.4 Hz, 2H), 7.32 (t, J=7.4 Hz, 2H), 7.22 (t, J=6.6 Hz, 1H), 5.28-5.15 (m, 2H), 4.32 (q, J=7.7 Hz, 2H), 3.90-3.80 (m, 1H), 3.75-3.64 (m, 1H), 3.28-3.23 (m, 1H), 1.34 (t, J=6.4 Hz, 3H), 1.20-1.07 (m, 6H).

### Example 17: Ethyl 4-[[(1S)-2-hydroxy-1-phenyl-ethyl]amino]-2-[(2-oxo-3,4-dihydro-1H-quinolin-6-yl)amino]pyrimidine-5-carboxylate—Compound 69

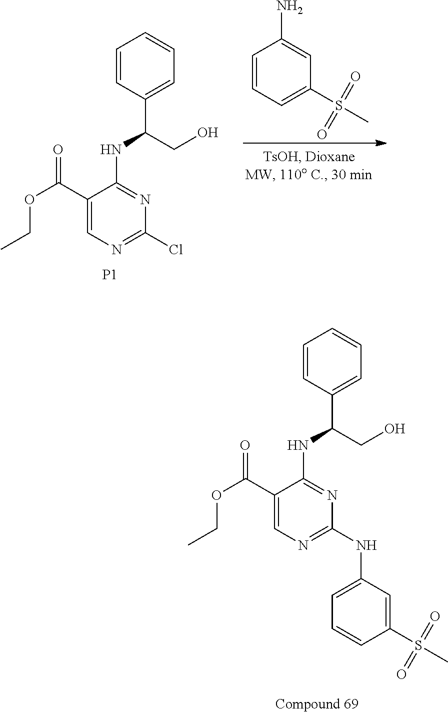

To a solution of ethyl 2-chloro-4-{[(1S)-2-hydroxy-1-phenylethyl]amino}pyrimidine-5-carboxylate P1 (100 mg, 0.31 mmol) in Dioxane (5 ml) was added 3-(methylsulfonyl)aniline (54 mg, 0.31 mmol) and TsOH (59 mg, 0.31 mmol). After cooling to room temperature, the reaction mixture was concentrated in vacuo, the residue was dissolved in EtOAc (20 ml), washed with water solution NaHCO₃—5% and dried over sodium sulfate. The organic layer was evaporated in vacuo and the crude product was purified by column chromatography (eluting with DCM/Et₂O) to provide Compound 69 as a solid. Yield: 83 mg, 58%. ¹H-NMR (400 MHz, dmso-d₆) δ: 10.12 (br s, 1H), 9.01 (d, J=7.8 Hz, 1H), 8.64 (s, 1H), 8.40 (s, 1H), 7.88-7.78 (m, 1H), 7.55-7.47 (m, 2H), 7.39 (d, J=8.1 Hz, 2H), 7.33 (t, J=7.6 Hz, 2H), 7.22 (t, J=7.4 Hz, 1H), 5.41-5.32 (m, 1H), 5.13 (t, J=5.1 Hz, 1H), 4.31 (q, J=7.0 Hz, 2H), 3.90-3.80 (m, 1H), 3.76-3.66 (m, 1H), 3.17 (s, 3H), 1.34 (t, J=7.3 Hz, 3H).

### Example 18: Ethyl 2-(4-carbamoyl-3-chloro-anilino)-4-[[(1S)-2-hydroxy-1-phenyl-ethyl]amino]pyrimidine-5-carboxylate—Compound 559

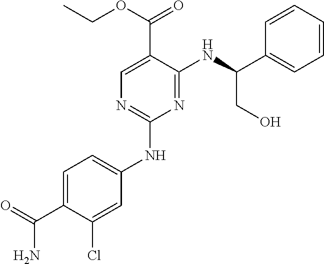 [SMILES: CCOC(=O)c1cnc(Nc2ccc(C(N)=O)c(Cl)c2)nc1N[C@H](CO)c1ccccc1]

The compound was synthesized according to the procedure described in Example 17. ¹H-NMR (400 MHz, dmso-d₆) δ: 10.01 (br. s, 1H), 9.08-8.96 (m, 1H), 8.63 (s, 1H), 7.80 (br. S, 1H), 7.71 (br. s, 1H), 7.52 (d, J=9.0 Hz, 1H), 7.45 (br. s, 1H), 7.42-7.30 (m, 5H), 7.27-7.18 (m, 1H), 5.29-5.22 (m, 1H), 5.21-5.13 (m, 1H), 4.31 (q, J=6.7 Hz, 2H), 3.90-3.78 (m, 1H), 3.76-3.65 (m, 1H), 1.34 (t, J=7.2 Hz, 3H).

### Example 19: N-ethyl-4-{[(1S)-2-hydroxy-1-phenylethyl]amino}-2-{[3-methyl-4-(methylsulfonyl)phenyl]amino}pyrimidine-5-carboxamide—Compound 156

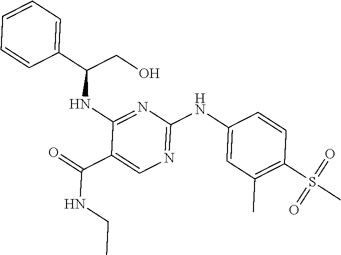 [SMILES: CCNC(=O)c1cnc(Nc2ccc(S(C)(=O)=O)c(C)c2)nc1N[C@H](CO)c1ccccc1]

A mixture of 4-{[(1S)-2-hydroxy-1-phenylethyl]amino}-2-{[3-methyl-4-(methylsulfonyl)phenyl]amino}pyrimidine-5-carboxylic acid (see [symbol] ) (0.07 g, 0.157 mmol), ethylamine hydrochloride (0.037 g, 0.340 mmol), HATU (0.066 g, 0.172 mmol), DIPEA (0.080 g, 0.626 mmol), and DMF (1 mL) was stirred at ambient temperature for 16 h, diluted with EtOAc (5 mL), washed with 10% aq. solution of citric acid, water, brine, dried under Na₂SO₄, and concentrated under reduced pressure. The residue was subjected to silica CC eluting with a mixture of DCM and Et₂O (2:1) to afford 0.034 g (47%) of the title compound as a white solid. ¹H NMR (400 MHz, DMSO-d₆): δ 9.85 (s, 1H), 9.80 (d, J=7.8 Hz, 1H), 8.56 (s, 1H), 8.37 (t, J=5.3 Hz, 1H), 7.17-7.66 (m, 2H), 7.59 (s, 1H), 7.37-7.21 (m, 5H), 5.25-5.20 (m, 1H), 5.08 (t, J=5.0 Hz, 1H), 3.82-3.77 (m, 1H), 3.72-3.67 (m, 1H), 3.30-3.25 (m, 2H), 3.13 (s, 3H), 2.54 (s, 3H), 1.14 (t, J=7.2 Hz, 3H).

### Example 20: 2-[(6-fluorobiphenyl-3-yl)amino]-4-{[(1S)-2-hydroxy-1-phenylethyl]amino}-N-methylpyrimidine-5-carboxamide—Compound 82

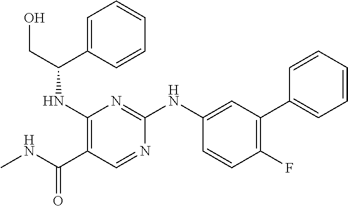 [SMILES: CNC(=O)c1cnc(Nc2ccc(F)c(-c3ccccc3)c2)nc1N[C@H](CO)c1ccccc1]

The compound was synthesized according to the procedure described in Example 19 using 2-[(6-fluorobiphenyl-3-yl)amino]-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride. ¹H NMR (400 MHz, DMSO-d₆): δ 9.79 (d, J=7.9 Hz, 1H), 9.54 (s, 1H), 8.48 (s, 1H), 8.32-8.28 (m, 1H), 7.82-7.80 (m, 1H), 7.58-7.40 (m, 6H), 7.26-7.11 (m, 6H), 5.18-5.01 (m, 2H), 3.78-3.75 (m, 1H), 3.62-3.58 (m, 1H), 2.76 (d, J=4.4 Hz, 3H).

### Example 21: 2-{[3-fluoro-4-(methylsulfonyl)phenyl]amino}-4-{[(1S)-2-hydroxy-1-phenylethyl]amino}-N-methylpyrimidine-5-carboxamide—Compound 105

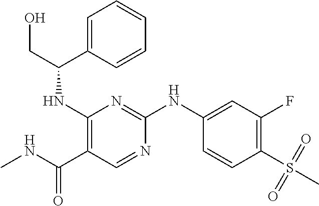

The compound was synthesized according to the procedure described in [symbol] using 2-{[3-fluoro-4-(methylsulfonyl)phenyl]amino}-4-{[(1S)-2-hydroxy-1-phenylethyl]-amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride. ¹H NMR (400 MHz, DMSO-d₆): δ 10.13 (s, 1H), 9.87-9.85 (m, 1H), 8.55 (s, 1H), 8.42-8.40 (m, 1H), 7.78 (d, J=13.3 Hz, 1H), 7.65 (t, J=8.3 Hz, 1H), 7.49 (d, J=8.3 Hz, 1H), 7.39-7.21 (m, 5H), 5.18-5.10 (m, 2H), 3.82-3.62 (m, 2H), 3.25 (s, 3H), 2.79-2.78 (m, 3H).

### Example 22: 4-{[(1S)-2-hydroxy-1-phenylethyl]amino}-N-methyl-2-[(2-methyl-1-oxo-1,2,3,4-tetrahydroisoquinolin-6-yl)amino]pyrimidine-5-carboxamide—Compound 43

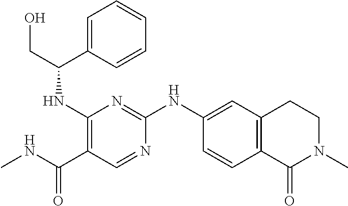

The compound was synthesized according to the procedure described in [symbol] using 4-{[(1S)-2-hydroxy-1-phenylethyl]amino}-2-[(2-methyl-1-oxo-1,2,3,4-tetrahydroisoquinolin-6-yl)amino]pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride. Yield is 90%, as a slight yellow solid. ¹H NMR (400 MHz, DMSO-d₆): 9.78 (d, J=7.6 Hz, 1H), 9.68 (s, 1H), 8.51 (s, 1H), 8.34-8.33 (m, 1H), 7.68 (d, J=9.3 Hz, 1H), 7.53-7.51 (m, 2H), 7.37-7.30 (m, 4H), 7.23-7.19 (m, 1H), 5.25-5.02 (m, 2H), 3.81-3.68 (m, 2H), 3.52-3.48 (m, 2H), 2.99 (s, 3H), 2.94-2.86 (m, 2H), 2.76 (d, J=4.5 Hz, 3H).

### Example 23: 4-{[(1S)-2-hydroxy-1-phenylethyl]amino}-N-methyl-2-[(2-methyl-3-oxo-1,2,3,4-tetrahydroisoquinolin-7-yl)amino]pyrimidine-5-carboxamide—Compound 44

The compound was synthesized according to the procedure described in [symbol] using 4-{[(1S)-2-hydroxy-1-phenylethyl]amino}-2-[(2-methyl-3-oxo-1,2,3,4-tetrahydroisoquinolin-7-yl)amino]pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride.

### Example 24: 2-{[3-chloro-4-(trifluoromethyl)phenyl]amino}-4-{[(1S)-2-hydroxy-1-phenylethyl]amino}-N-methylpyrimidine-5-carboxamide—Compound 132

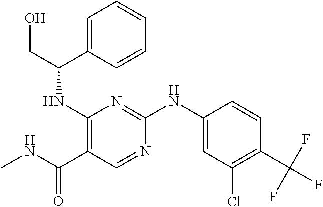 [SMILES: CNC(=O)c1cnc(Nc2ccc(C(F)(F)F)c(Cl)c2)nc1N[C@H](CO)c1ccccc1]

The compound was synthesized according to the procedure described in [symbol] using 2-{[3-chloro-4-(trifluoromethyl)phenyl]amino}-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride. ¹H NMR (400 MHz, DMSO-d₆): δ 9.99 (s, 1H), 9.84-9.83 (m, 1H), 8.55 (s, 1H), 8.40 (s, 1H), 8.03 (s, 1H), 7.70-7.62 (m, 2H), 7.40-7.21 (m, 5H), 5.21-5.10 (m, 2H), 3.82-3.66 (m, 2H), 2.78-2.77 (m, 3H).

### Example 25: 2-{[3-fluoro-4-(1-hydroxy-2-methylpropan-2-yl)phenyl]amino}-4-{[(1S)-2-hydroxy-1-phenylethyl]amino}-N-methylpyrimidine-5-carboxamide—Compound 63

The compound was synthesized according to the procedure described in [symbol] using 2-{[3-fluoro-4-(2-hydroxy-1,1-dimethylethyl)phenyl]amino}-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride.

### Example 26: Ethyl 2-[3-chloro-4-(methylcarbamoyl)anilino]-4-[[(1S)-2-hydroxy-1-phenyl-ethyl]amino]pyrimidine-5-carboxylate—Compound 560

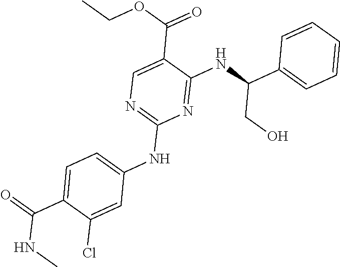 [SMILES: CCOC(=O)c1cnc(Nc2ccc(C(=O)NC)c(Cl)c2)nc1N[C@H](CO)c1ccccc1]

The compound was synthesized according to the procedure described in [symbol] . ¹H-NMR (400 MHz, dmso-d₆) δ: 10.02 (br s, 1H), 9.03 (d, J=8.4 Hz, 1H), 8.63 (s, 1H), 8.23-8.15 (m, 1H), 7.81 (br s, 1H), 7.56-7.48 (m, 1H), 7.42-7.31 (m, 4H), 7.30-7.19 (m, 2H), 5.28-5.20 (m, 1H), 5.19 (t, J=4.4 Hz, 1H), 4.31 (q, J=7.2 Hz, 2H), 3.88-3.79 (m, 1H), 3.75-3.65 (m, 1H), 2.75 (d, J=4.4 Hz, 3H), 1.34 (t, J=6.9 Hz, 3H).

### Example 27: 2-{[4-(1-hydroxy-2-methylpropan-2-yl)phenyl]amino}-4-{[(1S)-2-hydroxy-1-phenylethyl]amino}-N-methylpyrimidine-5-carboxamide—Compound 11

The compound was synthesized according to the procedure described in [symbol] using 2-{[4-(2-hydroxy-1,1-dimethylethyl)phenyl]amino}-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride.

### Example 28: 2-{[3-fluoro-4-(methylsulfonyl)phenyl]amino}-4-{[(1S)-2-hydroxy-1-phenylethyl]amino}-N-(propan-2-yl)pyrimidine-5-carboxamide—Compound 301

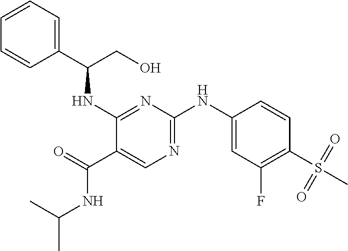 [SMILES: CC(C)NC(=O)c1cnc(Nc2ccc(S(C)(=O)=O)c(F)c2)nc1N[C@H](CO)c1ccccc1]

The compound was synthesized according to the procedure described in [symbol] using 2-{[3-fluoro-4-(methylsulfonyl)phenyl]amino}-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and isopropylamine instead of ethylamine hydrochloride. ¹H NMR (400 MHz, DMSO-d₆): δ 10.13 (s, 1H), 9.87-9.85 (m, 1H), 8.55 (s, 1H), 8.42-8.40 (m, 1H), 7.78 (d, J=13.3 Hz, 1H), 7.65 (t, J=8.3 Hz, 1H), 7.49 (d, J=8.3 Hz, 1H), 7.39-7.21 (m, 5H), 5.18-5.10 (m, 2H), 4.15-4.09 (m, 1H), 0.82-3.62 (m, 2H), 3.25 (s, 3H), 1.18 (d, J=5.4 Hz, 6H).

### Example 29: 2-{[3-chloro-4-(trifluoromethyl)phenyl]amino}-N-cyclopentyl-4-{[(1S)-2-hydroxy-1-phenylethyl]amino}pyrimidine-5-carboxamide—Compound 472

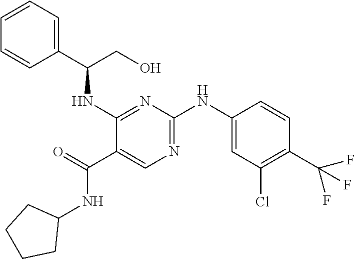 [SMILES: O=C(NC1CCCC1)c1cnc(Nc2ccc(C(F)(F)F)c(Cl)c2)nc1N[C@H](CO)c1ccccc1]

The compound was synthesized according to the procedure described in [symbol] using 2-{[3-chloro-4-(trifluoromethyl)phenyl]amino}-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and cyclopentylamine instead of ethylamine hydrochloride. ¹H NMR (400 MHz, DMSO-d₆): δ 10.02 (s, 1H), 9.82 (d, J=7.6 Hz, 1H), 8.61 (s, 1H), 8.20 (d, J=6.8 Hz, 1H), 8.04 (s, 1H), 7.68 (d, J=8.3 Hz, 1H), 7.62 (d, J=8.3 Hz, 1H), 7.39-7.19 (m, 5H), 5.22-5.18 (m, 1H), 5.11 (t, J=4.6 Hz, 1H), 4.26-4.21 (m, 1H), 3.82-3.77 (m, 1H), 3.70-3.64 (m, 1H), 1.93-1.86 (m, 2H), 1.75-1.67 (m, 2H), 1.59-1.50 (m, 4H).

### Example 30: 2-[(6-fluorobiphenyl-3-yl)amino]-4-{[(1S)-2-hydroxy-1-phenylethyl]-amino}-N-(propan-2-yl)pyrimidine-5-carboxamide—Compound 293

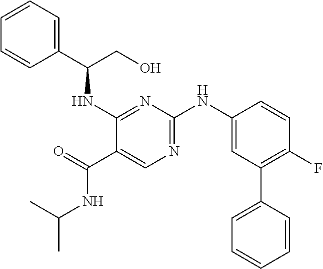 [SMILES: CC(C)NC(=O)c1cnc(Nc2ccc(F)c(-c3ccccc3)c2)nc1N[C@H](CO)c1ccccc1]

The compound was synthesized according to the procedure described in [symbol] using 2-[(6-fluorobiphenyl-3-yl)amino]-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and isopropylamine instead of ethylamine hydrochloride. ¹H NMR (400 MHz, DMSO-d₆): δ 9.78 (d, J=7.2 Hz, 1H), 9.56 (s, 1H), 8.55 (s, 1H), 8.04 (d, J=7.5 Hz, 1H), 7.82-7.80 (m, 1H), 7.58-7.40 (m, 6H), 7.26-7.11 (m, 6H), 5.18-5.14 (m, 1H), 4.10 (q, J=7.0 Hz, 1H), 3.75-3.71 (m, 1H), 3.60-3.54 (m, 1H), 1.17 (d, J=7.0 Hz, 6H).

### Example 31: N-ethyl-2-[(6-fluorobiphenyl-3-yl)amino]-4-{[(1S)-2-hydroxy-1-phenylethyl]amino}pyrimidine-5-carboxamide—Compound 189

The compound was synthesized according to the procedure described in [symbol] using 2-[(6-fluorobiphenyl-3-yl)amino]-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid. ¹H NMR (400 MHz, DMSO-d₆): δ 9.77 (d, J=7.2 Hz, 1H), 9.53 (s, 1H), 8.52 (s, 1H), 8.3-8.27 (m, 1H), 7.82-7.80 (m, 1H), 7.58-7.40 (m, 6H), 7.26-7.11 (m, 6H), 5.18-5.14 (m, 1H), 5.05-5.03 (m, 1H), 3.75-3.71 (m, 1H), 3.60-3.54 (m, 1H), 3.29-3.25 (m, 2H), 1.13 (t, J=7.2 Hz, 3H).

### Example 32: N-cyclopentyl-2-[(6-fluorobiphenyl-3-yl)amino]-4-{[(1S)-2-hydroxy-1-phenylethyl]amino}pyrimidine-5-carboxamide—Compound 440

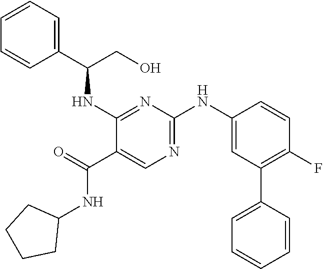 [SMILES: O=C(NC1CCCC1)c1cnc(Nc2ccc(F)c(-c3ccccc3)c2)nc1N[C@H](CO)c1ccccc1]

The compound was synthesized according to the procedure described in [symbol] using 2-[(6-fluorobiphenyl-3-yl)amino]-4-{[(1S)-2-hydroxy-1-phenylethyl]amino}pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and cyclopentylamine instead of ethylamine hydrochloride. ¹H NMR (400 MHz, DMSO-d₆): δ 9.76 (d, J=7.2 Hz, 1H), 9.56 (s, 1H), 8.55 (s, 1H), 8.09 (d, J=7.0 Hz, 1H), 7.84-7.82 (m, 1H), 7.58-7.40 (m, 6H), 7.26-7.11 (m, 6H), 5.18-5.14 (m, 1H), 4.27-4.22 (q, J=7.0 Hz, 1H), 3.75-3.71 (m, 1H), 3.60-3.54 (m, 1H), 1.93-1.86 (m, 2H), 1.75-1.67 (m, 2H), 1.59-1.50 (m, 4H).

### Example 33: 2-[(6-fluorobiphenyl-3-yl)amino]-4-{[(1S)-2-hydroxy-1-phenylethyl]-amino}-N-(2,2,2-trifluoroethyl)pyrimidine-5-carboxamide—Compound 499

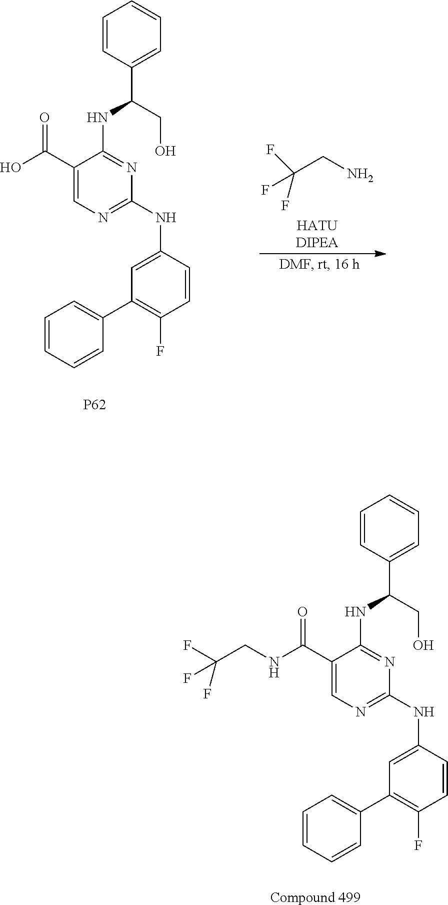 [SMILES: "P62" ; O=C(O)c1cnc(Nc2ccc(F)c(-c3ccccc3)c2)nc1N[C@H](CO)c1ccccc1 ; O=C(NCC(F)(F)F)c1cnc(Nc2ccc(F)c(-c3ccccc3)c2)nc1N[C@H](CO)c1ccccc1 ; NCC(F)(F)F ; "Compound 499"]

To a solution of P62 (70.0 mg, 0.157 mmol), trifluoroethylamine (15.5 mg, 0.172 mmol) and HATU (66.0 mg, 0.172 mmol) in DMF (1 ml) a DIPEA (80.0 mg, 0.626 mmol) was added, and mixture was stirred at rt for 16 h. Then a mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give Compound 499 (45 mg, 58% yield). ¹H NMR (400 MHz, DMSO-d₆): 9.67 (br., 1H), 9.69-9.57 (m, 1H), 8.86 (t, J=6.1 Hz, 1H), 8.61 (s, 1H), 7.81-7.79 (m, 1H), 7.56-7.43 (m, 6H), 7.26-7.12 (m, 6H), 5.18-5.16 (m, 1H), 5.08 (t, J=4.6 Hz, 1H), 4.15-4.00 (m, 2H), 3.77-3.72 (m, 1H), 3.61-3.56 (m, 1H).

### Example 34: 4-{[(1S)-2-hydroxy-1-phenylethyl]amino}-N-methyl-2-{[3-methyl-4-(methylsulfonyl)phenyl]amino}pyrimidine-5-carboxamide—Compound 65

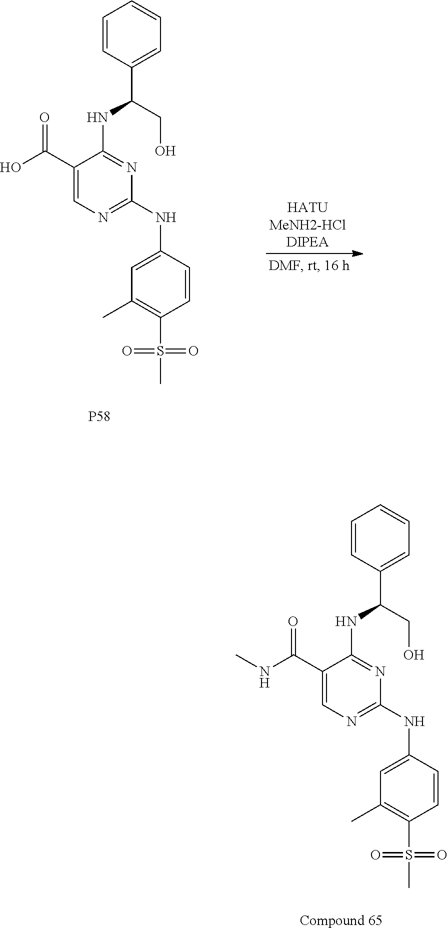 [SMILES: "P58" ; Cc1cc(Nc2ncc(C(=O)O)c(N[C@H](CO)c3ccccc3)n2)ccc1S(C)(=O)=O ; CNC(=O)c1cnc(Nc2ccc(S(C)(=O)=O)c(C)c2)nc1N[C@H](CO)c1ccccc1 ; "Compound 65"]

To a solution of P58 (70.0 mg, 0.157 mmol), methylamine hydrochloride (11.6 mg, 0.172 mmol) and HATU (66.0 mg, 0.172 mmol) in DMF (1 ml) a DIPEA (80.0 mg, 0.626 mmol) was added, and mixture was stirred at rt for 16 h. Then a mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give the Compound 65 (20 mg, 28% yield). ¹H NMR (400 MHz, DMSO-d₆): 9.96-9.92 (m, 2H), 8.52 (s, 1H), 8.43-8.40 (m, 1H), 7.73 (d, J=8.8 Hz, 1H), 7.74 (dd, J1-8.8 Hz, J2-2.0 Hz, 1H), 7.57 (s, 1H), 7.37-7.31 (m, 4H), 7.25-7.21 (m, 1H), 5.25-5.20 (m, 1H), 4.88-4.80 (m, 1H), 3.82-3.78 (m, 1H), 3.72-3.68 (m, 1H), 3.14 (s, 3H), 2.78 (d, J=4.3 Hz, 3H), 2.55 (s, 3H).

### Example 35: 4-{[(1S)-2-hydroxy-1-phenylethyl]amino}-2-{[3-methyl-4-(methylsulfonyl)phenyl]amino}-N-(propan-2-yl)pyrimidine-5-carboxamide—Compound 267

The compound was synthesized according to the procedure described in [symbol] using isopropylamine instead of ethylamine hydrochloride. ¹H NMR (400 MHz, DMSO-d₆): δ 9.86 (s, 1H), 9.78 (d, J=7.6 Hz, 1H), 8.59 (s, 1H), 8.12 (d, J=7.8 Hz, 1H), 7.71-7.65 (m, 2H), 7.37-7.20 (m, 5H), 5.24-5.20 (m, 1H), 5.10-5.06 (m, 1H), 4.11 (q, J=6.7 Hz, 1H), 3.82-3.77 (m, 1H), 3.73-3.67 (m, 1H), 3.31 (s, 3H), 2.54 (s, 3H), 1.17 (d, J=6.7 Hz, 6H).

### Example 36: 4-{[(1S)-2-hydroxy-1-phenylethyl]amino}-2-{[3-methyl-4-(methylsulfonyl)phenyl]amino}-N-(2,2,2-trifluoroethyl)pyrimidine-5-carboxamide—Compound 490

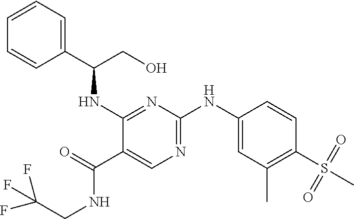 [SMILES: Cc1cc(Nc2ncc(C(=O)NCC(F)(F)F)c(N[C@H](CO)c3ccccc3)n2)ccc1S(C)(=O)=O]

The compound was synthesized according to the procedure described in [symbol] using 2,2,2-trifluoroethanamine instead of ethylamine hydrochloride. ¹H NMR (400 MHz, DMSO-d₆): δ 9.97 (s, 1H), 9.60 (d, J=8.2 Hz, 1H), 8.95 (t, J=8.3 Hz, 1H), 8.66 (s, 1H), 7.72-7.65 (m, 2H), 7.57 (s, 1H), 7.37-7.23 (m, 5H), 5.26-5.21 (m, 1H), 5.11 (t, J=7.9 Hz, 1H), 4.17-4.00 (m, 2H), 3.82-3.77 (m, 1H), 3.70-3.64 (m, 1H), 3.13 (s, 3H), 2.54 (s, 3H).

### Example 37: N-cyclopentyl-4-{[(1S)-2-hydroxy-1-phenylethyl]amino}-2-{[3-methyl-4-(methylsulfonyl)phenyl]amino}pyrimidine-5-carboxamide—Compound 434

The compound was synthesized according to the procedure described in [symbol] using cyclopentylamine instead of ethylamine hydrochloride. ¹H NMR (400 MHz, DMSO-d₆): δ 9.86 (s, 1H), 9.77 (d, J=7.6 Hz, 1H), 8.61 (s, 1H), 8.17 (d, J=7.0 Hz, 1H), 7.71-7.65 (m, 2H), 7.60 (s, 1H), 7.37-7.19 (m, 5H), 5.22-5.18 (m, 1H), 5.11 (t, J=4.6 Hz, 1H), 4.26-4.21 (m, 1H), 3.82-3.77 (m, 1H), 3.70-3.64 (m, 1H), 3.13 (s, 3H), 2.54 (s, 3H), 1.93-1.86 (m, 2H), 1.75-1.67 (m, 2H), 1.59-1.50 (m, 4H).

### Example 38: N-ethyl-4-{[(1S)-2-hydroxy-1-phenylethyl]amino}-2-[(2-methyl-1-oxo-1,2,3,4-tetrahydroisoquinolin-6-yl)amino]pyrimidine-5-carboxamide—Compound 119

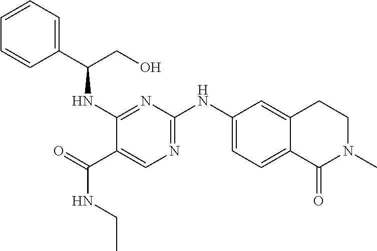 [SMILES: CCNC(=O)c1cnc(Nc2ccc3c(c2)CCN(C)C3=O)nc1N[C@H](CO)c1ccccc1]

The compound was synthesized according to the procedure described in [symbol] using 4-{[(1S)-2-hydroxy-1-phenylethyl]amino}-2-[(2-methyl-1-oxo-1,2,3,4-tetrahydroisoquinolin-6-yl)amino]pyrimidine-5-carboxylic acid instead of 4-{[(1S)-2-hydroxy-1-phenylethyl]amino}-2-{[3-methyl-4-(methylsulfonyl)phenyl]amino}-pyrimidine-5-carboxylic acid. ¹H NMR (400 MHz, DMSO-d₆): 9.79 (d, J=7.6 Hz, 1H), 9.69 (s, 1H), 8.54 (s, 1H), 8.35-8.33 (m, 1H), 7.68 (d, J=9.0 Hz, 1H), 7.53-7.51 (m, 2H), 7.37-7.30 (m, 4H), 7.23-7.19 (m, 1H), 5.25-5.02 (m, 2H), 3.81-3.68 (m, 2H), 3.52-3.48 (m, 2H), 3.3-3.26 (m, 2H), 2.99 (s, 3H), 2.92-2.86 (m, 2H), 1.14 (t, J=7.2 Hz, 3H).

### Example 39: 4-{[(1S)-2-hydroxy-1-phenylethyl]amino}-2-[(2-methyl-1-oxo-1,2,3,4-tetrahydroisoquinolin-6-yl)amino]-N-(propan-2-yl)pyrimidine-5-carboxamide—Compound 212

The compound was synthesized according to the procedure described in [symbol] using 4-{[(1S)-2-hydroxy-1-phenylethyl]amino}-2-[(2-methyl-1-oxo-1,2,3,4-tetrahydroisoquinolin-6-yl)amino]pyrimidine-5-carboxylic acid instead of 4-{[(1S)-2-hydroxy-1-phenylethyl]amino}-2-{[3-methyl-4-(methylsulfonyl)phenyl]amino}-pyrimidine-5-carboxylic acid and isopropylamine instead of ethylamine hydrochloride. ¹H NMR (400 MHz, DMSO-d₆): 9.77 (d, J=7.9 Hz, 1H), 9.71 (s, 1H), 8.57 (s, 1H), 8.09 (d, J=7.3 Hz, 1H), 7.68 (d, J=8.7 Hz, 1H), 7.53-7.51 (m, 2H), 7.37-7.30 (m, 4H), 7.23-7.20 (m, 1H), 5.25-5.02 (m, 2H), 4.16-4.07 (m, 1H), 3.81-3.68 (m, 2H), 3.52-3.48 (m, 2H), 2.99 (s, 3H), 2.92-2.86 (m, 2H), 1.17 (d, J=6.4 Hz, 6H).

### Example 40: N-cyclopentyl-4-{[(1S)-2-hydroxy-1-phenylethyl]amino}-2-[(2-methyl-1-oxo-1,2,3,4-tetrahydroisoquinolin-6-yl)amino]pyrimidine-5-carboxamide—Compound 389

The compound was synthesized according to the procedure described in [symbol] using 4-{[(1S)-2-hydroxy-1-phenylethyl]amino}-2-[(2-methyl-1-oxo-1,2,3,4-tetrahydroisoquinolin-6-yl)amino]pyrimidine-5-carboxylic acid instead of 4-{[(1S)-2-hydroxy-1-phenylethyl]amino}-2-{[3-methyl-4-(methylsulfonyl)phenyl]amino}-pyrimidine-5-carboxylic acid and cyclopentylamine instead of ethylamine hydrochloride. ¹H NMR (400 MHz, DMSO-d₆): 9.77 (d, J-8.1 Hz, 1H), 9.70 (s, 1H), 8.57 (s, 1H), 8.14 (d, J=7.5 Hz, 1H), 7.68 (d, J=8.7 Hz, 1H), 7.53-7.51 (m, 2H), 7.37-7.30 (m, 4H), 7.23-7.20 (m, 1H), 5.25-5.02 (m, 2H), 4.28-4.18 (m, 1H), 3.81-3.66 (m, 2H), 3.52-3.48 (m, 2H), 2.99 (s, 3H), 2.92-2.86 (m, 2H), 1.94-1.86 (m, 2H), 1.75-1.66 (m, 2H), 1.59-1.50 (m, 4H).

### Example 41: 4-{[(1S)-2-hydroxy-1-phenylethyl]amino}-N-methyl-2-[(1-oxo-1,2,3,4-tetrahydroisoquinolin-6-yl)amino]pyrimidine-5-carboxamide—Compound 9

The compound was synthesized according to the procedure described in [symbol] using 4-{[(1S)-2-hydroxy-1-phenylethyl]amino}-2-[(1-oxo-1,2,3,4-tetrahydroisoquinolin-6-yl)amino]pyrimidine-5-carboxylic acid instead of 4-{[(1S)-2-hydroxy-1-phenylethyl]amino}-2-{[3-methyl-4-(methylsulfonyl)phenyl]amino}pyrimidine-5-carboxylic acid and methylamine hydrochloride instead of ethylamine hydrochloride.

### Example 42: 2-{[3-fluoro-4-(methylsulfonyl)phenyl]amino}-4-{[(1S)-2-hydroxy-1-phenylethyl]amino}pyrimidine-5-carboxamide—Compound 31

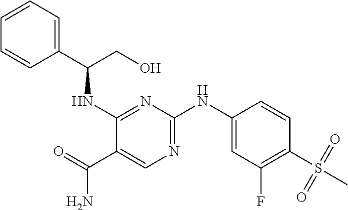 [SMILES: CS(=O)(=O)c1ccc(Nc2ncc(C(N)=O)c(N[C@H](CO)c3ccccc3)n2)cc1F]

A mixture of 2-{[3-fluoro-4-(methylsulfonyl)phenyl]amino}-4-{[(1S)-2-hydroxy-1-phenylethyl]amino}pyrimidine-5-carboxylic acid (0.5 g, 1.12 mmol), HOBt ammonia salt (0.85 g, 5.60 mmol), EDC (0.643 g, 3.36 mmol), and DMF (5 mL) a was stirred at ambient temperature for 48 h and treated with a saturated aq. solution of NaHCO₃. Formed precipitate was filtered off, washed with water, and dried at 70° C. to afford 0.41 g (82%) of the title compound as a slight-yellow solid. ¹H NMR (400 MHz, DMSO-d₆): 10.12 (s, 1H), 9.94 (d, J=7.5 Hz, 1H), 8.61 (s, 1H), 7.95-7.91 (m, 2H), 7.66-7.60 (m, 1H), 7.51-7.48 (m, 1H), 7.43-7.21 (m, 6H), 5.23-5.07 (m, 2H), 3.81-3.67 (m, 2H), 3.25 (s, 3H).

### Example 43: 4-{[(1S)-2-hydroxy-1-phenylethyl]amino}-2-{[3-methyl-4-(methylsulfonyl)phenyl]amino}pyrimidine-5-carboxamide—Compound 12

The compound was synthesized according to the procedure described in [symbol] using 4-{[(1S)-2-hydroxy-1-phenylethyl]amino}-2-{[3-methyl-4-(methylsulfonyl)phenyl]-amino}pyrimidine-5-carboxylic acid instead of 2-{[3-fluoro-4-(methylsulfonyl)phenyl]amino}-4-{[(1S)-2-hydroxy-1-phenylethyl]amino}pyrimidine-5-carboxylic acid.

### Example 44: 2-{[3-fluoro-4-(methylsulfonyl)phenyl]amino}-4-{[(1S)-2-hydroxy-1-phenylethyl]amino}pyrimidine-5-carbonitrile—Compound 2

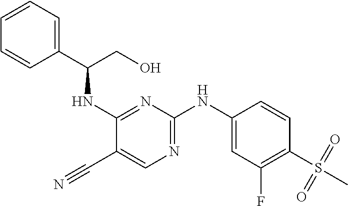 [SMILES: CS(=O)(=O)c1ccc(Nc2ncc(C#N)c(N[C@H](CO)c3ccccc3)n2)cc1F]

A solution of 2-{[3-fluoro-4-(methylsulfonyl)phenyl]amino}-4-{[(1S)-2-hydroxy-1-phenylethyl]amino}pyrimidine-5-carboxamide (0.135 g, 0.30 mmol) in TFAA (1 mL) was stirred at rt for 16 h and concentrated under reduced pressure. The residue was dissolved in DCM (10 mL), resulted solution was washed with saturated aq. solution of NaHCO₃, dried over Na₂SO₄, and concentrated under reduced pressure. The residue was subjected to silica CC eluting with a mixture DCM and Et₂O (10:1) to afford 0.023 g (21%) of the title compound as a white solid. ¹H NMR (400 MHz, DMSO-d₆): 10.28 (s, 1H), 9.21 (d, J=7.5 Hz, 1H), 8.63 (s, 1H), 7.80 (d, J=13.4 Hz, 1H), 7.65 (t, J=8.8 Hz, 1H), 7.50 (d, J=8.8 Hz, 1H), 7.39-7.21 (m, 5H), 5.25-5.21 (m, 2H), 3.87-3.68 (m, 2H), 3.25 (s, 3H).

### Example 45: 4-{[(1S)-2-hydroxy-1-phenylethyl]amino}-2-{[3-methyl-4-(methylsulfonyl)phenyl]amino}pyrimidine-5-carbonitrile—Compound 1

The compound was synthesized according to the procedure described in [symbol] using 4-{[(1S)-2-hydroxy-1-phenylethyl]amino}-2-{[3-methyl-4-(methylsulfonyl)phenyl]-amino}pyrimidine-5-carboxamide (see [symbol] ).

### Example 46: (2S)-2-{[2-{[3-fluoro-4-(methylsulfonyl)phenyl]amino}-5-(1,3,4-oxadiazol-2-yl)pyrimidin-4-yl]amino}-2-phenylethanol—Compound 161

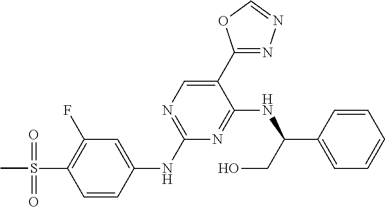 [SMILES: CS(=O)(=O)c1ccc(Nc2ncc(-c3nnco3)c(N[C@H](CO)c3ccccc3)n2)cc1F]

To a solution of acid obtained in [symbol] (100 mg, 0.22 mmol) in DCM (5 ml) was added (N-isocyanoimino)triphenyl phosphorane (136 mg, 0.44 mmol) at rt and stirred for overnight. After reaction completed the solvent was evaporated in vacuo and the crude product purified by column chromatography (eluting with Et₂O) and HPLC to obtained Compound 161 as solid. Yield 36 mg, 34%. ¹H-NMR (400 MHz, dmso-d₆) δ: 10.37 (br. s, 1H), 9.36 (s, 1H), 9.00 (d, J=8.3 Hz, 1H), 8.70 (s, 1H), 7.84 (d, J=14.0 Hz, 1H), 7.67 (t, J=9.0 Hz, 1H), 7.56-7.50 (m, 1H), 7.42 (d, J=6.9 Hz, 2H), 7.34 (t, J=7.8 Hz, 2H), 7.24 (t, J=6.3 Hz, 1H), 5.41-5.31 (m, 1H), 5.30-5.18 (m, 1H), 3.96-3.84 (m, 1H), 3.82-3.73 (m, 1H), 3.26 (s, 3H).

### Example 47: (2S)-2-{[2-{[3-fluoro-4-(methylsulfonyl)phenyl]amino}-5-(3-methyl-1,2,4-oxadiazol-5-yl)pyrimidin-4-yl]amino}-2-phenylethanol—Compound 270

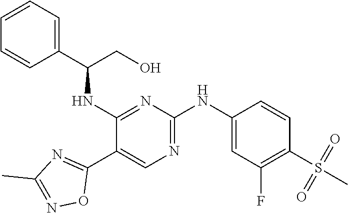 [SMILES: Cc1noc(-c2cnc(Nc3ccc(S(C)(=O)=O)c(F)c3)nc2N[C@H](CO)c2ccccc2)n1]

A mixture of 2-{[3-fluoro-4-(methylsulfonyl)phenyl]amino}-4-{[(1S)-2-hydroxy-1-phenylethyl]amino}pyrimidine-5-carboxylic acid (70 mg, 0.157 mmol), EDCI (30 mg, 0.157 mmol), HOBT hydrate (24 mg, 0.157 mmol), and DMF (1 mL) was stirred at ambient temperature for 1.5 h, and then a solution of N′-hydroxyethanimidamide (12 mg, 0.157 mmol) in DMF (0.5 ml) was added. The reaction mixture was stirred at 140° C. overnight, cooled down to ambient temperature, and concentrated under reduced pressure. The residue was subjected to silica CC eluting with a mixture of DCM and Et₂O to afford 11 mg (14%) of the title compound as a white solid. ¹H-NMR (400 MHz, DMSO-d₆) δ: 10.48 (s, 1H), 9.11 (d, J=7.1 Hz, 1H), 8.82 (s, 1H), 7.82 (d, J=13.2 Hz, 1H), 7.62 (t, J=8.6 Hz, 1H), 7.52 (d, J=9.0 Hz, 1H), 7.42 (d, J=7.4 Hz, 2H), 7.34 (t, J=7.2 Hz, 2H), 7.25 (t, J=6.5 Hz, 1H), 5.39-5.27 (m, 1H), 5.24 (t, J=4.8 Hz, 1H), 3.96-3.84 (m, 1H), 3.81-3.69 (m, 1H), 3.27 (s, 3H), 3.47 (s, 3H).

### Example 48: (2S)-2-{[2-{[3-methyl-4-(methylsulfonyl)phenyl]amino}-5-(3-methyl-1,2,4-oxadiazol-5-yl)pyrimidin-4-yl]amino}-2-phenylethanol—Compound 252

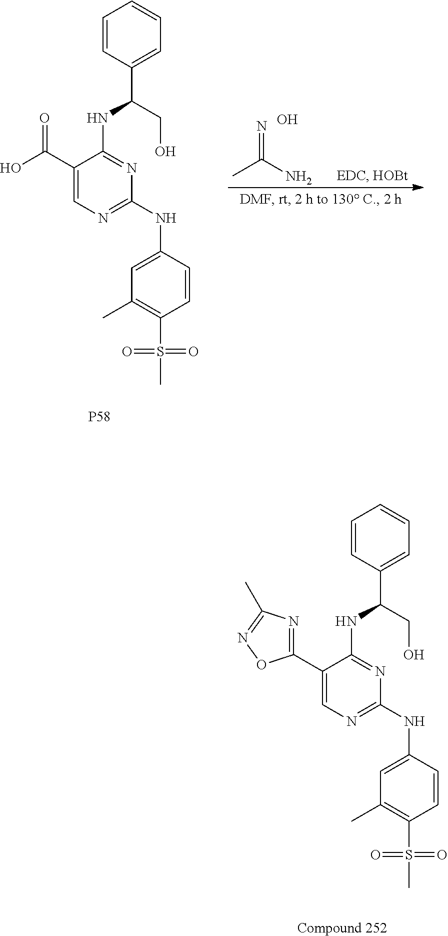

To a solution of P58 (100 mg, 0.226 mmol) and HOBt (33 mg, 0.248 mmol) in DMF (0.5 ml) an EDC (47 mg, 0.248 mmol) was added, and mixture stirred at rt for a 2 h. Then an amidoxime (18 mg, 0.258 mmol) was added and solution was stirred at 130° C. for a 2 h, then a mixture was cooled, diluted with EtOAc (5 ml), washed with 10% solution of NaHCO₃, brine, dried under Na₂SO₄ and concentrated to give a residue. The residue was purified by silica gel column chromatography (DCM:Et₂O=1:1) to give Compound 252 (8 mg, 7% yield). ¹H NMR (400 MHz, DMSO-d₆): δ 10.18 (s, 1H), 9.01 (d, J=6.8 Hz, 1H), 8.78 (s, 1H), 7.75-7.67 (m, 2H), 7.59 (s, 1H), 7.41-7.32 (m, 4H), 7.26-7.23 (m, 1H), 5.37-5.34 (m, 1H), 5.20-5.18 (m, 1H), 3.91-3.86 (m, 1H), 3.81-3.75 (m, 1H), 3.15 (s, 3H), 2.55 (s, 3H), 2.45 (s, 3H).

### Example 49: Ethyl 2-[(2-ethyl-1-oxo-3,4-dihydroisoquinolin-6-yl)amino]-4-[[(1S)-2-hydroxy-1-phenyl-ethyl]amino]pyrimidine-5-carboxylate—Compound 218

The compound was synthesized according to the procedure described in [symbol] .

### Example 50: Ethyl 4-[[(1S)-2-hydroxy-1-phenyl-ethyl]amino]-2-[(2-isopropyl-1-oxo-3,4-dihydroisoquinolin-6-yl)amino]pyrimidine-5-carboxylate—Compound 316

The compound was synthesized according to the procedure described in Preparation [symbol] .

### Example 51: 4-[[(1S)-2-hydroxy-1-phenyl-ethyl]amino]-N-(2-methoxyethyl)-2-(3-methyl-4-methylsulfonyl-anilino)pyrimidine-5-carboxamide—Compound 372

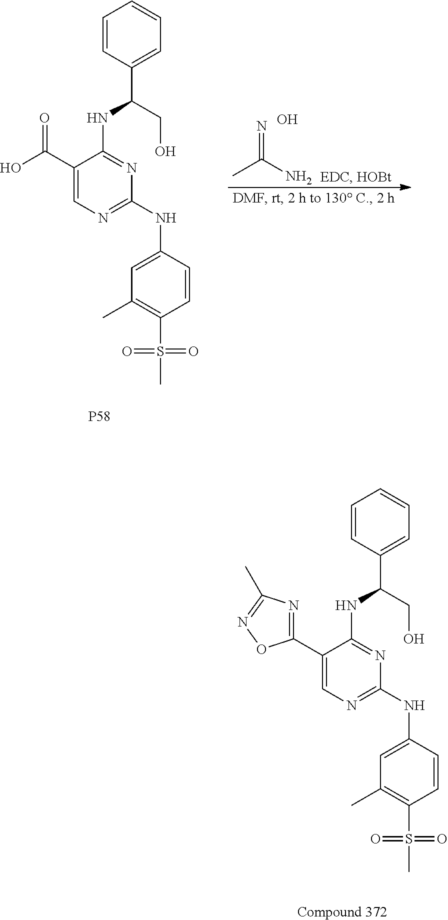

To a solution of P58 (70 mg, 0.157 mmol), 2-methoxyethylamine (12 mg, 0.160 mmol) and HATU (66 mg, 0.172 mmol) in DMF (1 ml) a DIPEA (80 mg, 0.626 mmol) was added, and mixture was stirred at rt for 16 h. Then a mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give Compound 372 (30 mg, 38% yield). ¹H NMR (400 MHz, DMSO-d₆): δ 9.68 (d, J=7.7 Hz, 1H), 8.28 (s, 1H), 7.88 (d, J=8.8 Hz, 1H), 7.55 (dd, J1=8.7 Hz, J2=2.1 Hz, 1H), 7.41-7.27 (m, 8H), 6.59-6.56 (m, 1H), 5.40-5.35 (m, 1H), 4.03-3.94 (m, 2H), 3.64-3.57 (m, 4H), 3.42 (s, 3H), 3.06 (s, 3H), 2.56 (s, 3H).

### Example 52: 4-[[(1S)-2-hydroxy-1-phenyl-ethyl]amino]-N,N-dimethyl-2-(3-methyl-4-methylsulfonyl-anilino)pyrimidine-5-carboxamide—Compound 157

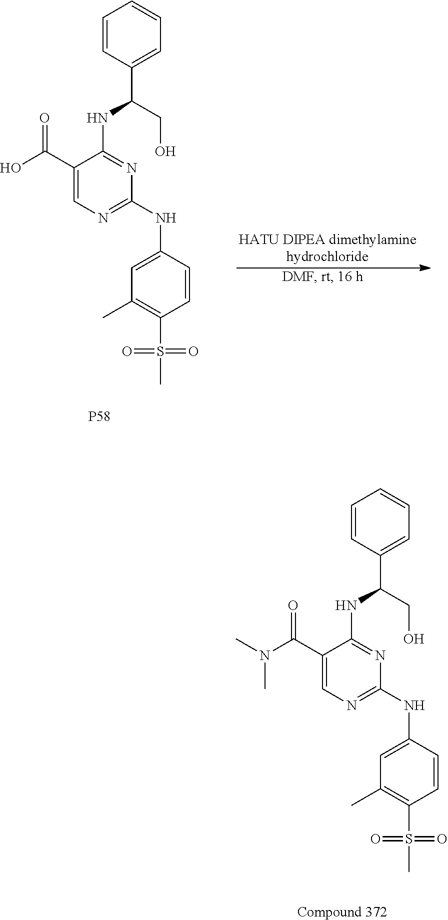 [SMILES: "P58" ; Cc1cc(Nc2ncc(C(=O)O)c(N[C@H](CO)c3ccccc3)n2)ccc1S(C)(=O)=O ; Cc1cc(Nc2ncc(C(=O)N(C)C)c(N[C@H](CO)c3ccccc3)n2)ccc1S(C)(=O)=O ; "Compound 372"]

To a solution of P58 (70 mg, 0.157 mmol), dimethylamine hydrochloride (13 mg, 0.160 mmol) and HATU (66 mg, 0.172 mmol) in DMF (1 ml) a DIPEA (80 mg, 0.626 mmol) was added, and mixture was stirred at rt for 16 h. Then the mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give Compound 157 (10 mg, 13% yield). ¹H NMR (400 MHz, DMSO-d₆): δ 9.72 (s, 1H), 8.10 (s, 1H), 7.88 (d, J=7.2 Hz, 1H), 7.71-7.70 (m, 2H), 7.58-7.56 (m, 1H), 7.40-7.30 (m, 4H), 7.24-7.20 (m, 1H), 5.25-5.20 (m, 1H), 5.10-5.00 (m, 1H), 3.80-3.75 (m, 1H), 3.72-3.67 (m, 1H), 3.13 (s, 3H), 3.04 (s, 6H), 2.54 (s, 3H).

### Example 53: 2-(3-fluoro-4-methylsulfonyl-anilino)-N-(2-hydroxyethyl)-4-[[(1S)-2-hydroxy-1-phenyl-ethyl]amino]-N-methyl-pyrimidine-5-carboxamide—Compound 402

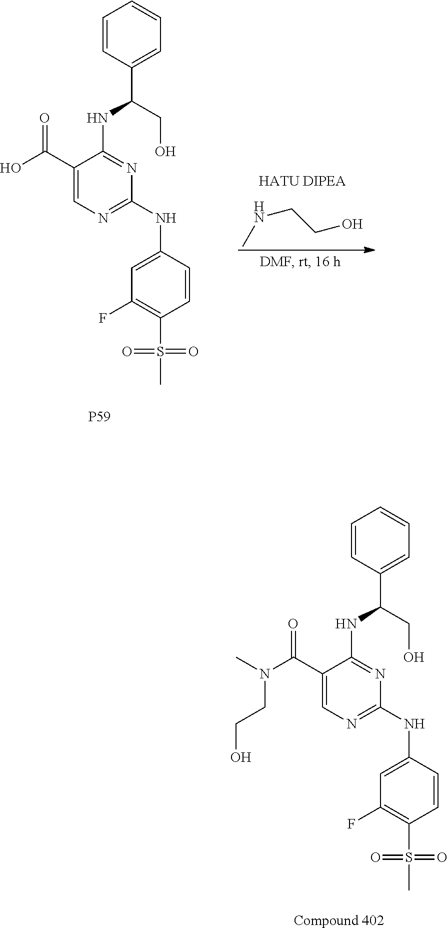

To a solution of P59 (70 mg, 0.157 mmol), N-methylethanolamine (12 mg, 0.172 mmol) and HATU (66 mg, 0.172 mmol) in DMF (1 ml) a DIPEA (80 mg, 0.626 mmol) was added, and mixture was stirred at rt for 16 h. Then a mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give Compound 402 (43 mg, 57% yield). ¹H NMR (400 MHz, DMSO-d₆): δ 10.01 (s, 1H), 8.15 (s, 1H), 7.82 (d, J=12.8 Hz, 1H), 7.69 (d, J=7.1 Hz, 1H), 7.62 (t, J=8.2 Hz, 1H), 7.48 (d, J=8.7 Hz, 1H), 7.41-7.39 (m, 2H), 7.31 (t, J=7.5 Hz, 2H), 7.23-7.20 (m, 1H), 5.21-5.16 (m, 1H), 5.06 (t, J=5.3 Hz, 1H), 4.90 (t, J=5.3 Hz, 1H), 3.79-3.44 (m, 6H), 3.24 (s, 3H), 3.05 (s, 3H).

### Example 54: [2-(3-fluoro-4-methylsulfonyl-anilino)-4-[[(1S)-2-hydroxy-1-phenyl-ethyl]amino]pyrimidin-5-yl]-pyrrolidin-1-yl-methanone—Compound 371

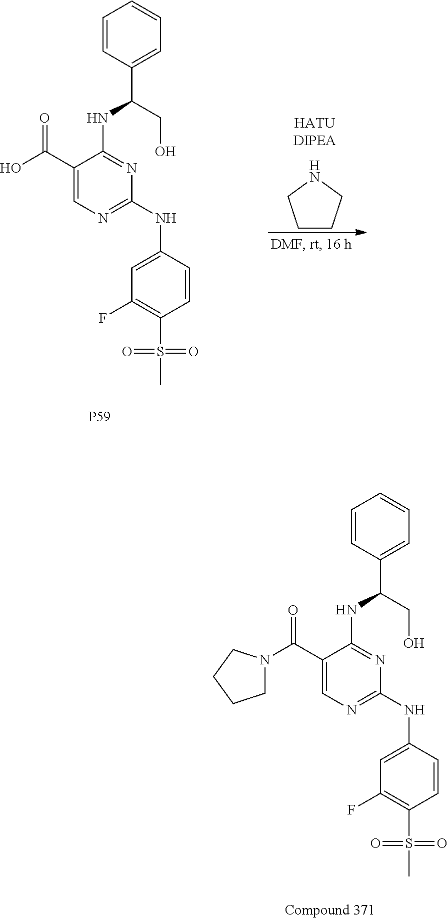 [SMILES: "P59" ; CS(=O)(=O)c1ccc(Nc2ncc(C(=O)O)c(N[C@H](CO)c3ccccc3)n2)cc1F ; CS(=O)(=O)c1ccc(Nc2ncc(C(=O)N3CCCC3)c(N[C@H](CO)c3ccccc3)n2)cc1F ; C1CCNC1 ; "Compound 371"]

To a solution of P59 (70 mg, 0.157 mmol), pyrrolidine (11 mg, 0.172 mmol) and HATU (66 mg, 0.172 mmol) in DMF (1 ml) a DIPEA (80 mg, 0.626 mmol) was added, and mixture was stirred at rt for 16 h. Then a mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give Compound 371 (38 mg, 48% yield). ¹H NMR (400 MHz, DMSO-d₆): δ 10.05 (s, 1H), 8.60 (d, J=7.7 Hz, 1H), 8.30 (s, 1H), 7.83 (d, J=14.1 Hz, 1H), 7.63 (t, J=8.4 Hz, 1H), 7.50 (d, J=9.0 Hz, 1H), 7.40-7.38 (m, 2H), 7.32 (t, J=7.6 Hz, 2H), 7.24-7.20 (m, 1H), 5.21-5.16 (m, 1H), 5.08 (t, J=4.9 Hz, 1H), 3.81-3.76 (m, 1H), 3.70-3.64 (m, 1H), 3.61-3.52 (m, 4H), 3.24 (s, 3H).

### Example 55: 2-(3-fluoro-4-methylsulfonyl-anilino)-4-[[(1S)-2-hydroxy-1-phenyl-ethyl]amino]-N-(2-methoxyethyl)-pyrimidine-5-carboxamide—Compound 401

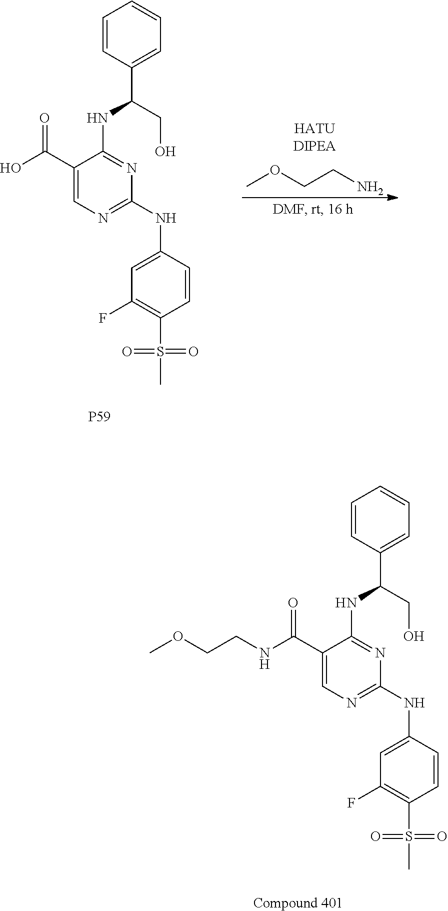

To a solution of P59 (70 mg, 0.157 mmol), 2-methoxyethylamine (12 mg, 0.160 mmol) and HATU (66 mg, 0.172 mmol) in DMF (1 ml) a DIPEA (80 mg, 0.626 mmol) was added, and mixture was stirred at rt for 16 h. Then a mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give Compound 401 (20 mg, 25% yield). ¹H NMR (400 MHz, DMSO-d₆): δ 10.14 (s, 1H), 9.83 (d, J=7.6 Hz, 1H), 8.60 (s, 1H), 8.49-8.46 (m, 1H), 7.81 (d, J=13.3 Hz, 1H), 7.63 (t, J=8.6 Hz, 1H), 7.48 (d, J=7.2 Hz, 1H), 7.40-4.31 (m, 4H), 7.24-7.21 (m, 1H), 5.21-5.16 (m, 1H), 5.08 (t, J=4.9 Hz, 1H), 3.81-3.76 (m, 1H), 3.70-3.64 (m, 1H), 3.50-3.39 (m, 4H), 3.29 (s, 3H), 3.24 (s, 3H).

### Example 56: 2-(3-fluoro-4-methylsulfonyl-anilino)-4-[[(1S)-2-hydroxy-1-phenyl-ethyl]amino]-N-(2-methoxyethyl)-pyrimidine-5-carboxamide—Compound 319

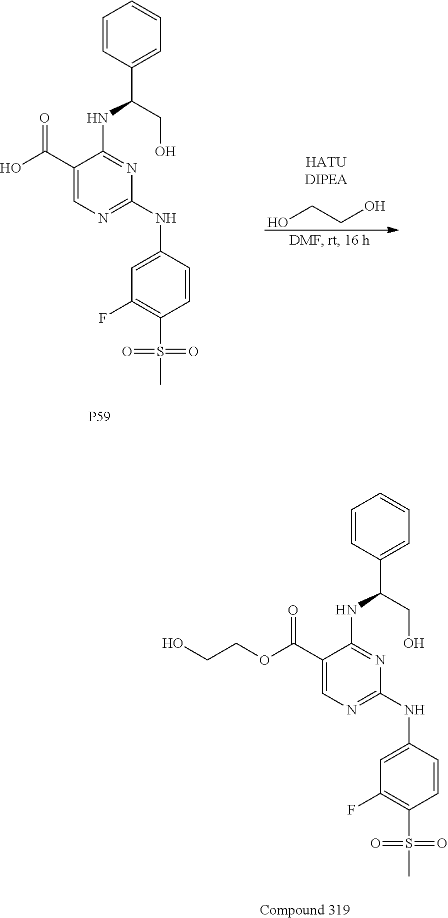

To a solution of P59 (100 mg, 0.223 mmol), ethylene glycol (70 mg, 1.121 mmol) and HATU (102 mg, 0.268 mmol) in DMF (1 ml) a DIPEA (86 mg, 0.669 mmol) was added, and mixture was stirred at rt for 16 h. Then a mixture was diluted with EtOAc (5 ml), a solution was washed with 10% solution of citric acid, water, brine, dried under Na₂SO₄ and concentrated to give a residue. The residue was purified by silica gel column chromatography (DCM:Et₂O=2:1) to give Compound 319 (15 mg, 14% yield). ¹H NMR (400 MHz, DMSO-d₆): δ 10.40 (s, 1H), 9.05 (d, J=7.0 Hz, 1H), 8.76 (s, 1H), 7.81 (d, J=13.1 Hz, 1H), 7.63 (t, J=8.8 Hz, 1H), 7.48 (d, J=8.6 Hz, 1H), 7.40-4.31 (m, 4H), 7.24-7.21 (m, 1H), 5.24-5.19 (m, 2H), 4.96 (t, J=5.6 Hz, 1H), 4.28 (t, J=4.8 Hz, 2H), 3.87-3.82 (m, 1H), 3.74-3.69 (m, 3H), 3.25 (s, 3H).

### Example 57: Ethyl 2-[4-(dimethylcarbamoyl)anilino]-4-[[(1S)-2-hydroxy-1-phenyl-ethyl]amino]pyrimidine-5-carboxylate—Compound 47

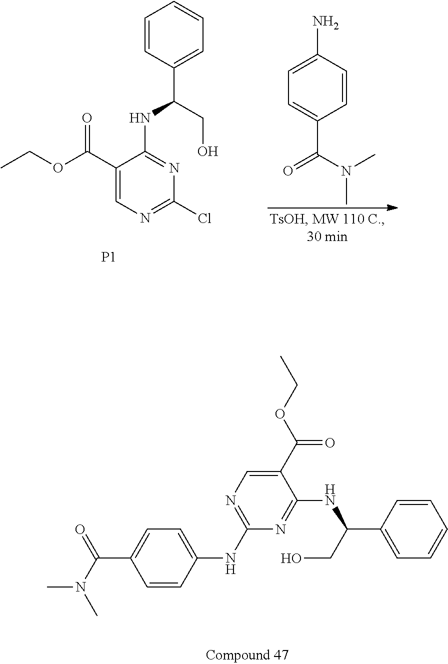 [SMILES: CCOC(=O)c1cnc(Cl)nc1N[C@H](CO)c1ccccc1 ; CN(C)C(=O)c1ccc(N)cc1 ; "P1" ; CCOC(=O)c1cnc(Nc2ccc(C(=O)N(C)C)cc2)nc1N[C@H](CO)c1ccccc1]

To a solution of ethyl 2-chloro-4-{[(1S)-2-hydroxy-1-phenylethyl]amino}pyrimidine-5-carboxylate P1 (98 mg, 0.305 mmol) and 4-amino-N,N-dimethylbenzamide (51 mg, 0.305 mmol) in a vial in dioxane (3 ml) a TsOH-H₂O (58 mg, 0.305 mmol) was added. A vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel CC (EtOAc:n-hexane=1:1) to give Compound 47 (33 mg, 24% yield). ¹H NMR (400 MHz, DMSO-d₆): δ 9.89 (s, 1H), 8.98 (d, J=7.2 Hz, 1H), 8.61 (s, 1H), 7.45 (d, J=8.4 Hz, 2H), 7.37-7.32 (m, 4H), 7.27-7.23 (m, 3H), 5.21-5.15 (m, 2H), 4.31 (q, J=6.8 Hz, 2H), 3.85-3.79 (m, 1H), 3.70-3.65 (m, 1H), 2.96 (s, 6H), 1.33 (t, J=7.0 Hz, 3H).

### Example 58: Ethyl 2-[3-chloro-4-(dimethylcarbamoyl)anilino]-4-[[(1S)-2-hydroxy-1-phenyl-ethyl]amino]pyrimidine-5-carboxylate—Compound 266

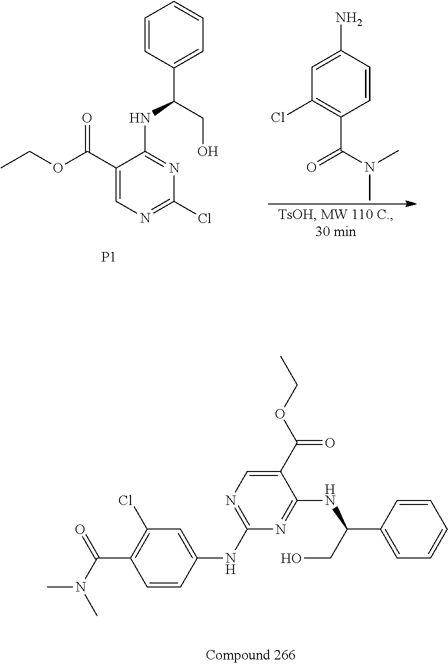 [SMILES: CCOC(=O)c1cnc(Cl)nc1N[C@H](CO)c1ccccc1 ; CN(C)C(=O)c1ccc(N)cc1Cl ; "P1" ; CCOC(=O)c1cnc(Nc2ccc(C(=O)N(C)C)c(Cl)c2)nc1N[C@H](CO)c1ccccc1]

To a solution of ethyl 2-chloro-4-{[(1S)-2-hydroxy-1-phenylethyl]amino}pyrimidine-5-carboxylate P1 (126 mg, 0.393 mmol) and 4-amino-2-chloro-N,N-dimethylbenzamide (78 mg, 0.393 mmol) in a vial in dioxane (3 ml) a TsOH-H₂O (75 mg, 0.393 mmol) was added. A vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel CC (EtOAc:n-hexane=1:1) to give Compound 266 (39 mg, 21% yield). ¹H NMR (400 MHz, DMSO-d₆): δ 10.01 (s, 1H), 9.02 (d, J=7.2 Hz, 1H), 8.63 (s, 1H), 7.84 (s, 1H), 7.55 (s, J=8.7 Hz, 1H), 7.38-7.30 (m, 4H), 7.23-7.16 (m, 2H), 5.25-5.22 (m, 1H), 5.18 (t, J=4.5 Hz, 1H), 4.31 (q, J=7.0 Hz, 2H), 3.88-3.81 (m, 1H), 3.72-3.67 (m, 1H), 2.99 (s, 3H), 2.78 (s, 3H), 1.34 (t, J=7.0 Hz, 3H).

### Example 59: (2S)-2-[[2-(3-methyl-4-methylsulfonyl-anilino)-5-nitro-pyrimidin-4-yl]amino]-2-phenyl-ethanol—Compound 18

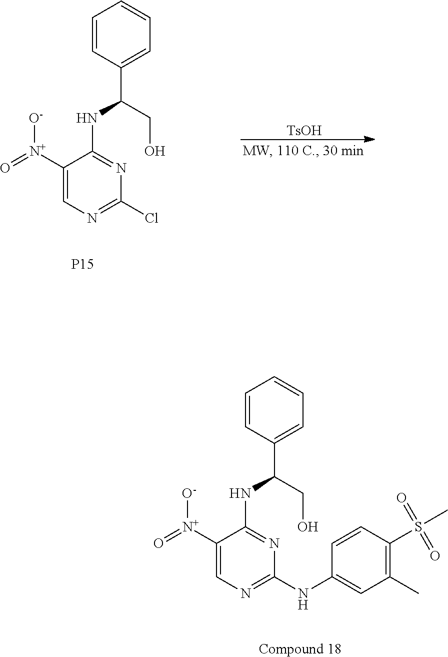

To a solution of P15 (100 mg, 0.339 mmol) and 4-sulfonylmethyl-3-methylaniline (63 mg, 0.339 mmol) in a vial in dioxane (3 ml) a TsOH-H₂O (64 mg, 0.339 mmol) was added. The vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel CC (EtOAc:n-hexane=1:1) to give Compound 18 (35 mg, 23% yield). ¹H NMR (400 MHz, DMSO-d₆): δ 10.62 (s, 1H), 9.23 (d, J=6.4 Hz, 1H), 9.06 (s, 1H), 7.74 (d, J=8.6 Hz, 1H), 7.64 (d, J=8.6 Hz, 1H), 7.54 (s, 1H), 7.42 (d, J=7.6 Hz, 2H), 7.34 (t, J=7.6 Hz, 2H), 7.27-7.23 (m, 1H), 5.37-5.33 (m, 1H), 5.29 (t, J=4.9 Hz, 1H), 3.91-3.79 (m, 2H), 3.16 (s, 3H), 2.55 (s, 3H).

### Example 60: Ethyl 2-(4-carbamoyl-3-methyl-anilino)-4-[[(1S)-2-hydroxy-1-phenyl-ethyl]amino]pyrimidine-5-carboxylate—Compound 10

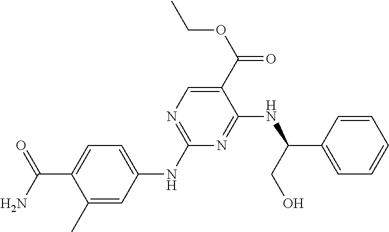 [SMILES: CCOC(=O)c1cnc(Nc2ccc(C(N)=O)c(C)c2)nc1N[C@H](CO)c1ccccc1]

The Compound 10 was synthesized according to the procedure described in [symbol] using P1 (50 mg, 0.16 mmol) and corresponding aniline. ¹H-NMR (400 MHz, dmso-d₆) δ: 9.79 (br s, 1H), 8.97 (d, J=8.1 Hz, 1H), 8.61 (br. s, 1H), 7.56 (br. s, 1H), 7.46-7.39 (m, 2H), 7.38-7.31 (m, 4H), 7.30-21 (m, 2H), 7.17 (br. s, 1H), 5.31-5.22 (m, 1H), 5.16 (t, J=5.2 Hz, 1H), 4.30 (q, J=7.0 Hz, 2H), 3.87-3.79 (m, 1H), 3.76-3.67 (m, 1H), 2.33 (s, 3H), 1.33 (t, J=6.8 Hz, 3H).

### Example 61: Ethyl 4-[[(1S)-2-hydroxy-1-phenyl-ethyl]amino]-2-[(2-oxo-3,4-dihydro-1H-quinolin-6-yl)amino]pyrimidine-5-carboxylate—Compound 46

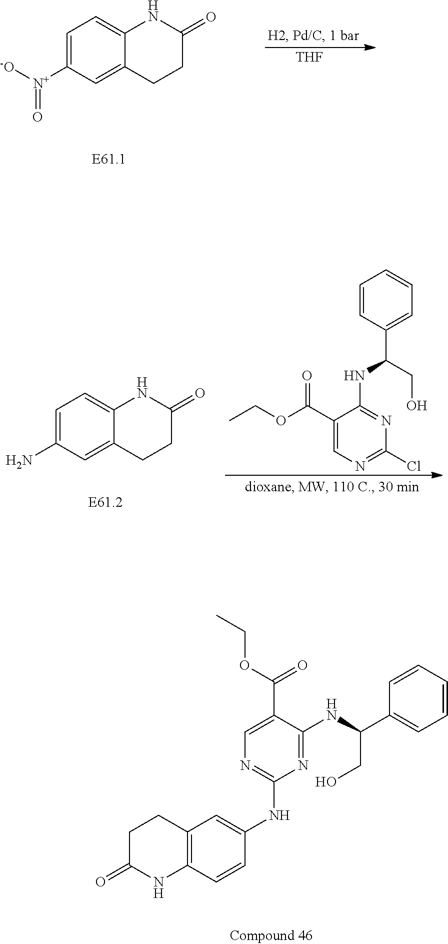

Synthesis of E62.2. To a solution of E62.1 (1.00 g, 5.20 mmol) in THF (10 ml) a 10% Pd/C (100 mg) was added, and mixture was stirred under H₂ (1 bar) at rt for a 16 h. Then a mixture was filtered through a pad of Celite, filtrate was concentrated to give a residue. A residue was recrystallized from Et₂O, a solid was filtered to give E62.2 (0.75 g, 89% yield). ¹H NMR (400 MHz, DMSO-d₆): δ 9.62 (s, 1H), 6.54 (d, J=8.2 Hz, 1H), 6.38-6.33 (m, 2H), 4.70 (br., 2H), 2.70 (t, J=7.8 Hz, 2H), 2.33 (t, J=7.2 Hz, 2H).

Synthesis of Compound 46. To a solution of E62.2 (100 mg, 0.617 mmol) and ethyl 2-chloro-4-{[(1S)-2-hydroxy-1-phenylethyl]amino}pyrimidine-5-carboxylate P1 (200 mg, 0.617 mmol) in a vial in dioxane (3 ml) a TsOH-H₂O (117 mg, 0.617 mmol) was added. The vial was capped and irradiated at 110° C. for a 30 min. A reaction mixture was diluted with EtOAc (10 ml), washed with 10% solution of NaHCO₃, brine, dried under Na₂SO₄ and concentrated to give a residue. The residue was purified by silica gel CC (EtOAc:n-hexane=1:1) to give Compound 46 (105 mg, 38% yield). ¹H NMR (400 MHz, DMSO-d₆): δ 9.93 (s, 1H), 9.63-9.56 (m, 1H), 8.93-8.88 (m, 1H), 8.56 (s, 1H), 7.33-7.23 (m, 7H), 6.71 (d, J=8.6 Hz, 1H), 5.23-5.20 (m, 1H), 5.13-5.10 (m, 1H), 4.28 (q, J=7.0 Hz, 2H), 3.82-3.78 (m, 1H), 3.72-3.68 (m, 1H), 2.82-2.76 (m, 2H), 2.43-2.39 (m, 2H), 1.32 (t, J=7.0 Hz, 3H).

### Example 62: Ethyl 6-[5-[2-(3-fluoro-4-methylsulfonyl-anilino)-4-[[(1S)-2-hydroxy-1-phenyl-ethyl]amino]pyrimidin-5-yl]-1,3,4-oxadiazol-2-yl]hexanoate—Compound 562

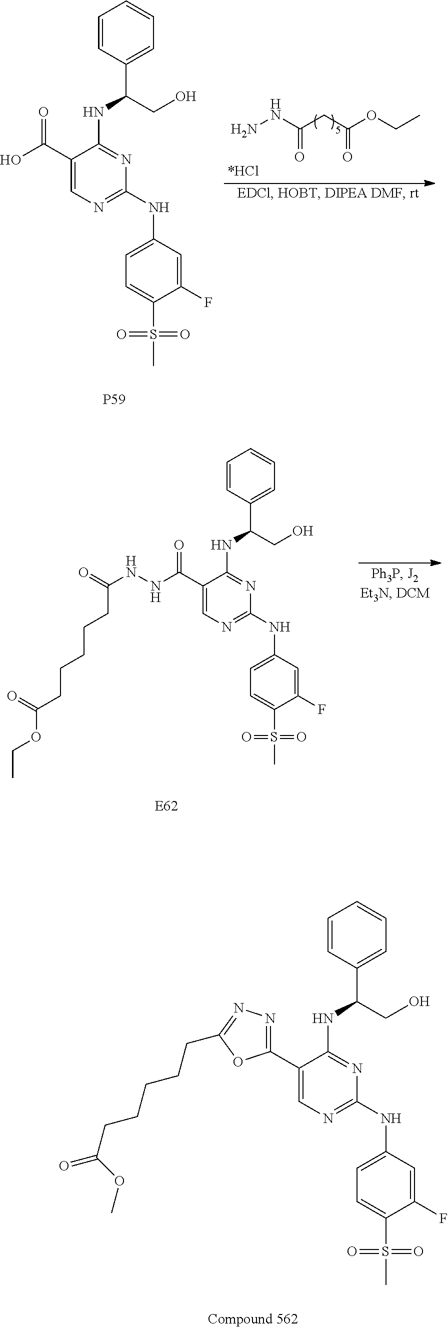

Synthesis of E62. To a solution of acid P59 (0.3 g, 0.00067 mol) in DMF (5 ml) was added ethyl 7-hydrazino-7-oxoheptanoate hydrochloride (0.18 g, 0.00074 mol), EDCI (0.15 g, 0.0008 mol), HOBT (0.12 g, 0.0008 mol) and DIPEA (0.35 ml, 0.002 mol). The reaction mixture was stirred at rt for overnight. The reaction mixture was diluted with water, the formed solid was collected by filtration, washed with water, and purified by column chromatography (DCM/Et₂O). The yield of E62 was 0.3 g, 72%. ¹H-NMR (400 MHz, dmso-d₆) δ: 10.25-10.18 (m, 2 NH), 9.78 (s, 1H), 9.59 (d, J=7.5 Hz, 1H), 8.66 (s, 1H), 7.86-7.77 (m, 1H), 7.65 (t, J=8.5 Hz, 1H) 7.52-7.46 (m, 1H), 7.39-7.29 (m, 4H), 7.26-7.20 (m, 1H), 5.24-5.16 (m, 1H), 5.14 (t, J=4.9 Hz, 1H), 4.09-3.99 (m, 2H), 3.86-3.76 (m, 1H), 3.72-3.64 (m, 1H), 3.25 (s, 3H), 2.29 (t, J=7.1 Hz, 2H), 2.20 (t, J=6.5 Hz, 2H), 1.62-1.50 (m, 4H), 1.34-1.27 (m, 2H), 1.20-1.14 (m, 3H).

Synthesis of Compound 562. To a solution of PPh₃ (0.23 g, 0.0014 mol) in DCM (10 ml) was added J2 (0.36 g, 0.0014 mol) at 0° C. and the reaction mixture was stirred at 0° C. for 10 min. Et₃N (0.5 ml, 0.0028 mol) was added and the reaction mixture was stirred at rt for 10 min. After that compound E62 (0.3 g, 0.0047 mol) in DCM (3 ml) was added at 0° C. and the reaction mixture was stirred at rt for overnight. The reaction mixture was diluted with water solution Na₂S₂O₅/NaHCO₃ (1:1), the organic layer was separated, evaporated under pressure and residue was purified by CC (DCM). The yield of Compound 562 was 0.18 g, 62%. ¹H-NMR (400 MHz, dmso-d₆) δ: 10.38 (br s, 1H), 8.98 (d, J=6.7 Hz, 1H), 8.65 (s, 1H), 7.83 (d, J=13.0 Hz, 1H), 7.67 (t, J=8.7 Hz, 1H) 7.55-7.49 (m, 1H), 7.41 (d, J=8.2 Hz, 2H), 7.34 (d, J=6.5 Hz, 2H), 7.24 (d, J=5.7 Hz, 1H), 5.39-5.29 (m, 1H), 5.24 (t, J=4.6 Hz, 1H), 4.05 (q, J=7.1 Hz, 2H), 3.95-3.85 (m, 1H), 3.81-3.70 (m, 1H), 3.26 (s, 3H), 2.94 (t, J=7.6 Hz, 2H), 2.32 (t, J=7.8 Hz, 2H), 1.85-1.73 (m, 2H), 1.66-1.53 (m, 2H), 1.47-1.37 (m, 2H), 1.17 (t, J=7.1 Hz, 3H).

### Example 63: Ethyl 6-[5-[4-[[(1S)-2-hydroxy-1-phenyl-ethyl]amino]-2-(3-methyl-4-methylsulfonyl-anilino)pyrimidin-5-yl]-1,3,4-oxadiazol-2-yl]hexanoate—Compound 561

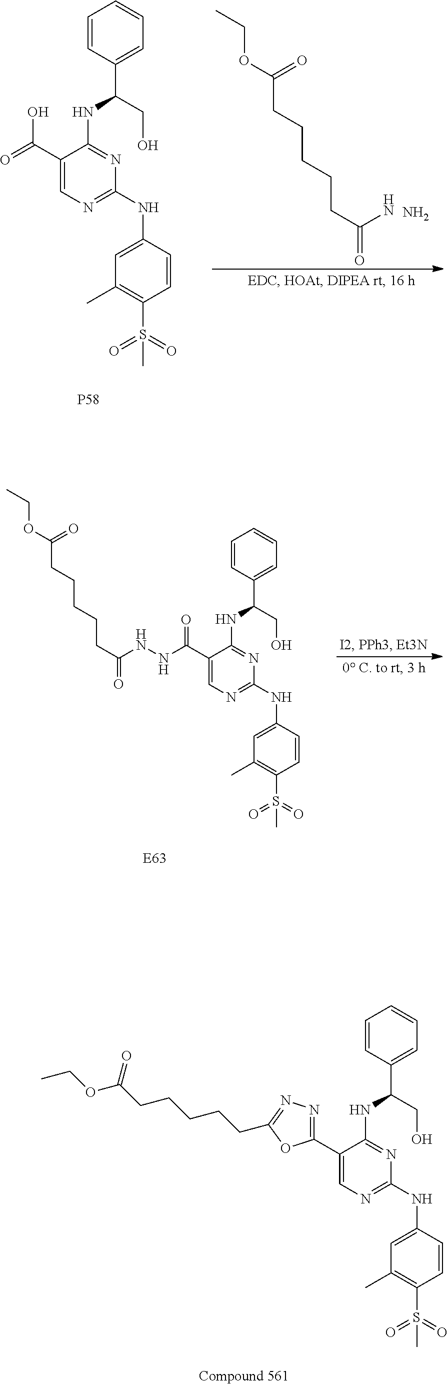

Synthesis of E63. To a solution of P58 (200.0 mg, 0.452 mmol), DIPEA (160.0 mg, 1.240 mmol) and HOAt (68.0 mg, 0.497 mmol) in DMF (0.3 ml) an EDC (95.0 mg, 0.497 mmol) was added, and mixture was stirred at rt for a 30 min. Then a hydrazide hydrochloride (119.0 mg, 0.497 mmol) was added, and mixture was stirred at rt for a 16 h. The mixture was diluted with water, solid was filtered, washed with water, and dried on air to give E63 (220 mg, 74% yield). ¹H NMR (400 MHz, DMSO-d₆): 10.15 (s, 1H), 9.91 (s, 1H), 9.74 (s, 1H), 9.52-9.50 (m, 1H), 8.63 (s, 1H), 7.73-7.66 (m, 2H), 7.57 (s, 1H), 7.36-7.31 (m, 4H), 7.24-7.21 (m, 1), 5.26-5.22 (m, 1H), 5.10-5.08 (m, 1H), 4.05 (q, J=7.0 Hz, 2H), 3.82-3.78 (m, 1H), 3.72-3.67 (m, 1H), 3.13 (s, 3H), 2.55 (s, 3H), 2.29 (t, J=7.6 Hz, 2H), 2.19 (t, J=7.0 Hz, 2H), 1.58-1.52 (m, 4H), 1.36-1.28 (m, 2H), 1.18 (t, J=7.0 Hz, 3H).

Synthesis of Compound 561. To a solution of PPh₃ (184 mg, 0.703 mmol) in DCM (2 ml) at 0° C. a I₂ (178 mg, 0.703 mmol) was added, and mixture was stirred at 0° C. for a 10 min, then a Et₃N (142 mg, 1.406 mmol) was added at 0° C. and mixture was stirred at rt for a 10 min. The mixture was cooled to 0° C. and a solution compound E63 (220 mg, 0.351 mmol) in DCM (1 ml) was added and mixture was stirred at rt for a 3 h. Then mixture was diluted with solution NaHCO₃ (5 ml) and Na₂S₂O₅ (5 ml), organic layer was washed with water, dried under Na₂SO₄, and concentrated to give a residue. The residue was purified by silica gel CC (DCM:THF=5:1) to give Compound 561 (90 mg, 41% yield). ¹H NMR (400 MHz, DMSO-d₆): 10.08 (s, 1H), 8.89 (d, J=7.5 Hz, 1H), 8.61 (d, J=1.3 Hz, 1H), 7.74-7.68 (m, 2H), 7.60 (s, 1H), 7.41-7.32 (m, 4H), 7.25-7.22 (m, 1H), 5.40-5.36 (m, 1H), 5.22-5.19 (m, 1H), 4.04 (q, J=7.0 Hz, 2H), 3.91-3.88 (m, 1H), 3.80-3.76 (m, 1H), 3.15 (s, 3H), 2.93 (t, J=7.0 Hz, 2H), 2.55 (s, 3H), 2.31 (t, J=6.9 Hz, 2H), 1.82-1.77 (m, 2H), 1.63-1.55 (m, 2H), 1.44-1.36 (m, 2H), 1.16 (t, J=7.0 Hz, 3H).

### Example 64: Ethyl 4-[[(1S)-2-hydroxy-1-phenyl-ethyl]amino]-2-[(3-isopropyl-2,5-dioxo-3,4-dihydro-1H-1,4-benzodiazepin-7-yl)amino]pyrimidine-5-carboxylate—Compound 467

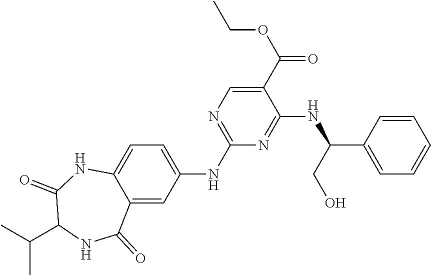 [SMILES: CCOC(=O)c1cnc(Nc2ccc3c(c2)C(=O)NC(C(C)C)C(=O)N3)nc1N[C@H](CO)c1ccccc1]

The compound 467 was synthesized according to the procedure described in [symbol] using P1 (50 mg, 0.16 mmol) and corresponding aniline. ¹H-NMR (400 MHz, dmso-d₆) δ: 10.19 (d, J=7.1 Hz, 1H), 9.89 (br s, 1H), 8.98 (br s, 1H), 8.62-8.52 (m, 2H), 8.27-8.08 (m, 1H), 7.67-7.51 (m, 1H), 7.42-7.27 (m, 4H), 7.25-7.17 (m, 1H), 6.96 (d, J=8.6 Hz, 1H), 5.41-5.26 (m, 1H), 5.18-5.07 (m, 1H), 4.30 (q, J=8.0 Hz, 2H), 3.87-3.77 (m, 1H), 3.74-3.63 (m, 1H), 3.27-3.16 (m, 1H), 2.06-1.86 (m, 1H), 1.33 (t, J=6.7 Hz, 3H), 1.03-0.85 (m, 6H).

### Example 65: Ethyl 4-[[(1S)-2-hydroxy-1-phenyl-ethyl]amino]-2-[3-methyl-4-(methylcarbamoyl)anilino]pyrimidine-5-carboxylate—Compound 48

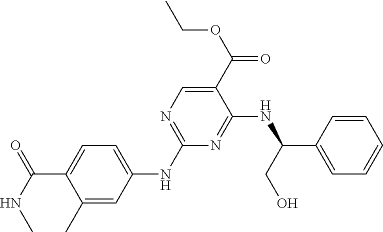 [SMILES: CCOC(=O)c1cnc(Nc2ccc(C(=O)NC)c(C)c2)nc1N[C@H](CO)c1ccccc1]

The Compound 48 was synthesized according to the procedure described in [symbol] using P1 (50 mg, 0.16 mmol) and corresponding aniline. ¹H-NMR (400 MHz, dmso-d₆) δ: 9.78 (br s, 1H), 8.95 (d, J=7.7 Hz, 1H), 8.61 (s, 1H), 8.0 (d, J=4.0 Hz, 1H), 7.48-7.40 (m, 2H), 7.39-7.30 (m, 4H), 7.28-7.17 (m, 2H), 5.32-5.23 (m, 1H), 5.15 (t, J=5.2 Hz, 1H), 4.30 (q, J=7.0 Hz, 2H), 3.89-3.79 (m, 1H), 3.78-3.66 (m, 1H), 2.74 (d, J=4.4 Hz, 3H), 2.29 (s, 3H), 1.33 (t, J=7.2 Hz, 3H).

### Example 66: Ethyl 2-[4-(dimethylcarbamoyl)-3-methyl-anilino]-4-[[(1S)-2-hydroxy-1-phenyl-ethyl]amino]pyrimidine-5-carboxylate—Compound 125

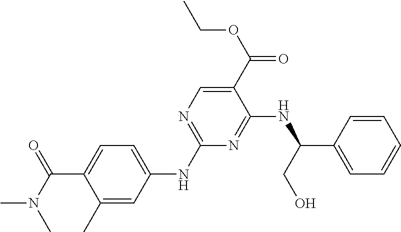 [SMILES: CCOC(=O)c1cnc(Nc2ccc(C(=O)N(C)C)c(C)c2)nc1N[C@H](CO)c1ccccc1]

The compound 125 was synthesized according to the procedure described in [symbol] using P1 (50 mg, 0.16 mmol) and corresponding aniline. ¹H-NMR (400 MHz, dmso-d₆) δ: 9.77 (br. s, 1H), 8.96 (d, J=7.0 Hz, 1H), 8.60 (s, 1H), 7.49-7.42 (m, 2H), 7.39-7.28 (m, 4H), 7.26-7.17 (m, 1H), 6.99 (d, J=8.9 Hz, 1H), 5.31-5.22 (m, 1H), 5.14 (t, J=4.9 Hz, 1H), 4.30 (q, J=7.0 Hz, 2H), 3.88-3.78 (m, 1H), 3.76-3.67 (m, 1H), 2.99 (s, 3H), 2.76 (s, 3H), 2.13 (s, 3H), 1.33 (t, J=6.9 Hz, 3H).

### Example 67: Ethyl 2-[4-(dimethylcarbamoyl)-3-methoxy-anilino]-4-[[(1S)-2-hydroxy-1-phenyl-ethyl]amino]pyrimidine-5-carboxylate—Compound 247

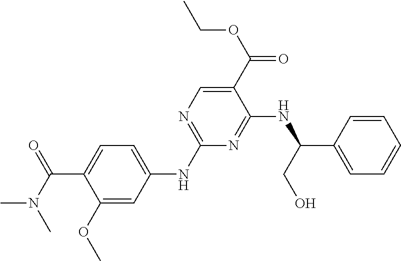 [SMILES: CCOC(=O)c1cnc(Nc2ccc(C(=O)N(C)C)c(OC)c2)nc1N[C@H](CO)c1ccccc1]

To a solution of Compound 62 ( [symbol] ) (60 mg, 0.13 mmol) and DIPEA (68 μL, 0.39 mmol) in THF (2 ml) were added dimethylamine (9 mg, 0.20 mmol) and TBTU (63 mg, 0.20 mmol). The mixture was stirred at 25° C. for 3 h. After reaction completed the solvent was evaporated in vacuo and the crude product purified by column chromatography (eluting with Et₂O) to obtained Compound 247 as a solid. Yield 25 mg, 40%. ¹H-NMR (400 MHz, dmso-d₆) δ: 9.79 (br. s, 1H), 8.94 (d, J=7.6 Hz, 1H), 8.62 (s, 1H), 7.41-7.27 (m, 6H), 7.26-7.19 (m, 1H), 6.99 (d, J=8.3 Hz, 1H), 5.35-5.26 (m, 1H), 5.14 (t, J=5.8 Hz, 1H), 4.30 (q, J=7.0 Hz, 2H), 3.87-3.79 (m, 1H), 3.77-3.68 (m, 4H), 2.95 (s, 3H), 2.76 (s, 3H), 1.33 (t, J=7.2 Hz, 3H).

### Biological Assays

### Example A. Primary Assay Used to Determine Potency of HPK1 Enzymatic Activity Inhibition

Compound activity was determined using recombinant HPK1 protein and MBP Substrate (both Promega, Cat #V6398) in an in vitro enzymatic reaction. The enzymatic assay used to determine activity was a Luminescence assay using a Microplate Reader ClarioStar Plus. The enzymatic reaction was carried out in assay buffer (40 mM TRIS-HCl PH 7.4-7.6, 20 mM MgCl₂, 0.05 mM DTT, 0.1 mg/ml BSA). The compounds were dispensed on a 384 well Diamond Well Plate (Axigen, Cat #P-384-120SQ-C-S) using the Biomek FX liquid handling system at 100× solutions of compounds in DMSO. 2×HPK1-MBP mix (final concentration 0.64 ng/μl of HPK1 and 45 ng/μl of MBP) was prepared in 1× Assay buffer and 5.5 μl of mixture per well was added into 384 w white Reaction plate with NBS (Corning, Cat #4513). 5.5 μl of MBP substrate w/o HPK1 in 1× buffer was used for negative control. Plates were centrifuged for 1 min at 100 g. Next step the Compounds were added to Reaction plate using Biomek station via following steps: 1 μl of 100× compounds (in DMSO) were mixed thoroughly with 49 μl of 2×10 μM ATP in Assay Buffer, then 5.5 μl of this mixture was added to Reaction plate with 5.5 μl of HPK1-MBP mix. Plates were centrifuged for 1 min at 100 g and incubated for 1 hour at room temperature. Next 3 μL of ADP-Glo reagent (Promega, ADP-Glo™ Kinase Assay, Cat #V9102) per well was added. Plates were incubated for 30 minutes at room temperature. Then 6 μL of Kinase detection reagent (Promega, ADP-Glo™ Kinase Assay, Cat #V9102) per well was added and the Luminescence was measured using Microplate Reader. The % inhibition was then used to calculate the IC₅₀ values. The IC₅₀ values are shown in Table A, wherein “A” corresponds to IC₅₀<50.0 nM, “B” corresponds to 50.0 nM≤IC₅₀<200.0 nM, “C” 200.0 nM≤IC₅₀<500.0 nM, and “D” corresponds to 500.0 nM≤IC₅₀.

TABLE A
Primary HPK1 inhibition

| Compound | |
|---|---|
| # | HPK1 IC₅₀ (nM) |
| 2 | D |
| 10 | B |
| 18 | C |
| 19 | A |
| 31 | B |
| 42 | D |
| 43 | B |
| 45 | A |
| 46 | A |
| 47 | B |
| 48 | D |
| 62 | C |
| 65 | B |
| 68 | A |
| 69 | C |
| 82 | B |
| 86 | A |
| 89 | A |
| 97 | D |
| 105 | B |
| 119 | A |
| 122 | A |
| 125 | C |
| 132 | D |
| 156 | B |
| 157 | C |
| 161 | A |
| 166 | A |
| 167 | C |
| 168 | B |
| 183 | A |
| 189 | C |
| 195 | C |
| 210 | C |
| 211 | B |
| 212 | B |
| 218 | B |
| 228 | A |
| 247 | C |
| 248 | D |
| 252 | B |
| 266 | C |
| 267 | B |
| 270 | A |
| 274 | C |
| 293 | C |
| 298 | D |
| 299 | D |
| 300 | B |
| 301 | C |
| 316 | D |
| 319 | A |
| 371 | D |
| 372 | C |
| 388 | D |
| 389 | B |
| 401 | B |
| 402 | D |
| 434 | C |
| 440 | D |
| 467 | D |
| 472 | D |
| 490 | A |
| 499 | C |
| 558 | A |
| 559 | A |
| 560 | B |
| 561 | B |
| 563 | A |
| 564 | A |
| 565 | A |
| 566 | A |
| 567 | D |
| 568 | B |
| 569 | A |
| 570 | A |
| 571 | A |
| 572 | A |
| 573 | A |
| 574 | D |
| 575 | A |
| 576 | B |
| 577 | D |
| 578 | A |
| 579 | A |
| 580 | A |
| 582 | B |
| 583 | A |
| 584 | A |
| 585 | A |
| 586 | A |
| 587 | A |

### Example B. IL-2 Release Jurkat Assay

Jurkat cells (ATCC, USA) (100,000 cells/well in a 96 well flat-bottom plate (Greiner, #655061) were cultured in RPMI 1640 supplemented with 10% FBS at 37° C., 5% CO₂ in a humidified cell culture incubator for 0.5-1 hours. Then 10 μL/well 15× compound (or DMSO) was added in duplicates. Cells were incubated with compound in a humidified cell culture incubator for 0.5-1 hours. After the incubation, 10 μL/well of 15× human anti-CD3 HIT3a antibody (BioLegend) together with 10 μL/well of 15× human anti-CD28 antibody (Invitrogen) were added to stimulate Jurkat cells for 24 hours. Additionally, Jurkat cells with DMSO were incubated in the absence of anti-CD3 to evaluate its activation effect. After 24 hours of stimulation, supernatants were collected and IL-2 levels in supernatants were assessed using IL-2 ELISA kit (Vector-Best, Russia). The IL-2 concentrations are shown in Table B, wherein “A” corresponds to IL-2<5.0 pg/mL, “B” corresponds to 5.0 pg/mL≤IL-2<25.0 pg/mL, “C” corresponds to 25.0 pg/mL≤IL-2<35.0 pg/mL, and “D” corresponds to 35.0 pg/mL≤IL-2.

TABLE B
IL-2 data from the Jurkat functional assay at 1.5 μM of tested compounds
and activation with 400 ng/mL aCD3 + 400 ng/mL aCD28.

| | |
|---|---|
| Compound | IL-2 (pg/mL) |

| # | no stimulation | stimulated |
|---|---|---|
| Vehicle | A | B |
| 19 | | B |
| 31 | | C |
| 43 | | D |
| 65 | | D |
| 68 | | D |
| 82 | | C |
| 86 | | C |
| 105 | | C |
| 119 | | D |
| 122 | | C |
| 156 | | C |
| 166 | | C |
| 189 | | C |
| 195 | | C |
| 210 | | D |
| 212 | | C |
| 267 | | D |
| 293 | | D |
| 301 | | A |
| 389 | | C |
| 434 | | B |
| 490 | | B |
| 499 | | C |
| 564 | | B |
| 568 | | A |
| 569 | | C |
| 570 | | B |
| 571 | | C |
| 572 | | B |

The IL-2 concentrations are shown in Tables C, D, E, F and G wherein “A” corresponds to IL-2<50 pg/mL, “B” corresponds to 50 pg/mL≤IL-2<100 pg/mL, “C” corresponds to 100 pg/mL≤IL-2<150 pg/mL, and “D” corresponds to 150 pg/mL≤IL-2.

TABLE C
IL-2 data from the Jurkat functional assay at 6 μM of tested compounds
and activation with 400 ng/mL aCD3 + 400 ng/mL aCD28.

| | |
|---|---|
| Compound | IL-2, pg/mL |

| # | no stimulation | stimulated |
|---|---|---|
| Vehicle | A | A |
| 18 | | D |
| 45 | | B |
| 47 | | B |
| 69 | | B |
| 157 | | C |
| 161 | | C |
| 167 | | B |
| 168 | | A |
| 211 | | B |
| 218 | | D |
| 252 | | B |
| 266 | | C |
| 270 | | A |
| 274 | | B |
| 300 | | A |
| 319 | | D |
| 372 | | C |
| 401 | | A |

TABLE D
IL-2 data from the Jurkat functional assay at 6 μM of tested compounds
and activation with 400 ng/mL aCD3 + 400 ng/mL aCD28.

| | |
|---|---|
| Compound | IL-2, pg/mL |

| # | no stimulation | stimulated |
|---|---|---|
| Vehicle | A | B |
| 10 | | D |
| 46 | | D |
| 125 | | D |
| 558 | | D |
| 559 | | D |
| 560 | | D |
| 561 | | B |

TABLE E
IL-2 data from the Jurkat functional assay at 1.5 μM of tested compounds
and activation with 400 ng/mL aCD3 + 400 ng/mL aCD28.

| | |
|---|---|
| Compound | IL-2, pg/mL |

| # | no stimulation | stimulated |
|---|---|---|
| Vehicle | A | A |
| 228 | | B |
| 566 | | D |
| 573 | | C |
| 575 | | C |

TABLE F
IL-2 data from the Jurkat functional assay at 1.0 μM of tested compounds
and activation with 400 ng/mL aCD3 + 400 ng/mL aCD28.

| | |
|---|---|
| Compound | IL-2, pg/mL |

| # | no stimulation | stimulated |
|---|---|---|
| Vehicle | A | A |
| 579 | | A |
| 582 | | B |
| 583 | | B |
| 584 | | A |

TABLE G
IL-2 data from the Jurkat functional assay at 1.0 μM of tested compounds
and activation with 400 ng/mL aCD3 + 400 ng/mL aCD28.

| | |
|---|---|
| Compound | IL-2, pg/mL |

| # | no stimulation | stimulated |
|---|---|---|
| 578 | | C |
| 581 | | D |
| 585 | | D |
| 586 | | D |
| 587 | | D |

### Example C. Pharmacokinetics Studies

Pharmacokinetic (PK) study in mice: Male CD-1 mice obtained from Charles River GmbH (Sulzfeld, Germany) with body weight ranging between 20 g to 30 g were used in the PK studies. Group of 12 mice were given a 2 mg/kg intravenous bolus (IV) dose of test article as a solution in 20% HP-beta-CD, and another group of 9 mice were given a 10 mg/kg oral (PO) dose of test article as a solution or suspension in 80% PEG400, 10% TPGS and 10% ethanol.

Blood samples (˜200 μL per time point) were collected twice from each animal—from orbital sinus and by cardiopuncture at 0.083, 0.25, 0.5, 1, 2, 4, 8 and 24 hours after IV administration, and at 0.5, 1, 2, 4, 8 and 24 hours after PO administration, 3 mice/time point. Blood samples were collected in tubes with Na-EDTA 0.5M solution (1:10), and centrifuged for 10 min at 10,000 rpm at 2 to 8° C. to harvest plasma. Plasma samples were stored at −80° C. until LC/MS/MS analysis.

Concentration in each plasma sample was determined by a non-validated LC/MS/MS method. Data is acquired using multiple reaction monitoring (MRM) with specific transitions monitored for each compound.

Pharmacokinetic Analysis: PK parameters were calculated by non-compartmental methods as described in Gibaldi and Perrier (Gibaldi and Perrier, 1982) using Phoenix® WinNonlin® version 6.3 (Certara L.P.). Following PO administration, percent bioavailability (Fabs) was determined by dividing the dose normalized mean area under the plasma concentration-time curve, extrapolated to the last time point (AUClast) obtained following PO dose by the mean dose normalized AUC of the animals dosed by IV injection. All PK parameters are presented as mean±standard deviation (SD).

PK studies were conducted in mice for exemplary compounds of Formula (I). The percent bioavailability (Fabs) values determined are listed in Table H for certain compounds, together with the dosage, vehicle and form used in the studies.

TABLE H

| | Fabs, | Dose | | |
|---|---|---|---|---|
| Compound | % | (mg/kg) | Vehicle | Form |
| 161 | 95.1 | 10 | 20% HP-b-CD | Solution |
| 270 | 76.5 | 10 | 80% PEG400, 10% TPGS and 10% ethanol | Solution |
| 86 | 83.6 | 10 | 80% PEG400, 10% TPGS and 10% ethanol | Solution |
| 558 | 16.3 | 10 | 80% PEG400, 10% TPGS and 10% ethanol | Solution |
| 252 | 19.9 | 10 | 80% PEG400, 10% TPGS and 10% ethanol | Solution |
| 228 | 11.4 | 10 | 80% PEG400, 10% TPGS and 10% ethanol | Solution |
| 19 | 16.3 | 10 | 80% PEG400, 10% TPGS and 10% ethanol | Solution |
| 563 | 9.2 | 10 | 80% PEG400, 10% TPGS and 10% ethanol | Solution |
| 572 | 5.7 | 10 | 80% PEG400, 10% TPGS and 10% ethanol | Solution |
| 571 | 8.0 | 10 | 80% PEG400, 10% TPGS and 10% ethanol | Solution |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula I: 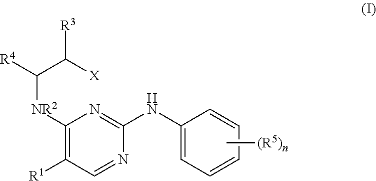
or a pharmaceutically acceptable salt, solvate, enantiomer, stereoisomer, or a tautomer thereof, wherein:
X is H, halogen, or OH, provided that when R⁴ is unsubstituted phenyl and X is H then R¹ is C(O)OR⁶;
R¹ is selected from —CN, —NO₂, —C(O)NHR⁶, C(O)N(R⁶)₂, —C(O)OR⁶, —S(O)₂C₁₋₆ alkyl or a monocyclic heteroaryl comprising one or more heteroatoms independently selected from N, S, and O, wherein the heteroaryl is optionally substituted with one or more substituents selected from —OH, oxo, halogen, C₁₋₄ alkoxy, C₁₋₆ alkyl, C₂₋₆ alkenyl, or 4-7 membered monocyclic heterocycloalkyl, wherein the alkyl or alkoxy is further optionally substituted with one or more substituents selected from halogen, —NH₂, —N(C₁-C₆ alkyl)₂, —OH, —COOC₁₋₄ alkyl, —COOH, —CONH₂ or 4-7 membered monocyclic heterocycloalkyl;
R² is H or C₁₋₄ alkyl; or
R² and R⁸ together with the atoms to which they are attached and any intervening atoms, form a 5- to-6-membered heterocycle;
R³ is H; or
R³ and R⁸ together with the atoms to which they are attached and any intervening atoms, form a 5- to-6-membered cycloalkyl;
R⁴ is selected from —(CH₂)ₘ-aryl, —(CH₂)ₘ-heteroaryl, heterocyclyl, wherein the aryl, heteroaryl, or heteroaryl is optionally substituted with one or more R⁸;
each R⁵ is independently selected from halogen, C₁₋₆ alkyl, C₁₋₆ alkoxy, C₁₋₆ halogenalkyl, oxy C₁₋₆ alkyl, —S(O)₂—C₁₋₆ alkyl, —S(O)₂—C₂₋₆ alkenyl, —C(O)NH₂, —C(O)NH(C₁₋₆ alkyl), —C(O)N(C₁₋₆ alkyl)₂, —CH₂C(O)NH₂, —CH₂C(O)NH(C₁₋₆ alkyl), —CH₂C(O)N(C₁₋₆ alkyl)₂, —NHC(O)CH₃, aryl, heteroaryl; or
two R⁵ together with the atoms to which they are attached and any intervening atoms, form 6-7 membered heterocyclyl, or 5-7 membered heteroaryl, containing at least one heteroatom selected from N, O, S; wherein the cycloalkyl or heterocyclyl is optionally substituted with one or more R⁷;
each R⁶ is independently selected from H, OH, C₁₋₆ alkyl, —S(O)₂—C₁₋₆ alkyl, C₃₋₈ cycloalkyl, heterocyclyl, or heteroaryl, wherein the alkyl, alkoxy, heteroaryl, or heterocyclyl is optionally substituted with one or more R⁹; or
two R⁶ together with the atoms to which they are attached and any intervening atoms, form 4-8-membered heterocycle;
each R⁷ is independently selected from OH, halogen, C₁₋₆ alkyl, aryl, oxy C₁₋₆ alkyl, —CH₂OC(O)C₁₋₆ alkyl, or —CH₂OCH₂CH₂Si(CH₃)₃;
each R⁸ is independently selected from halogen, OH, NH₂, C₁₋₆ alkyl, —OC₁₋₆ alkyl, —NHC₁₋₆ alkyl, —N(C₁₋₆ alkyl)₂;
each R⁹ is independently selected from halogen, OH, NH₂, C₁₋₆ alkyl, C₁₋₆ alkoxy, C₁₋₆ haloalkyl, C₂₋₆ alkenyl, C₂₋₆ alkynyl, C₁₋₆ haloalkyl, C₃₋₈ cycloalkyl, heterocyclyl, aryl, or heteroaryl; and
m and n are integers independently selected from 0, 1, 2, 3, 4, 5 and 6;
wherein:
aryl is cyclic, aromatic hydrocarbon groups that have 1 to 3 aromatic rings fused or connected each other via single bond;
heteroaryl is a monovalent monocyclic or polycyclic aromatic radical of 5 to 24 ring atoms, containing one or more ring heteroatoms selected from N, O, S, P, Se, or B, the remaining ring atoms being C;
heterocyclyl is a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms independently selected from O, N, S, P, Se, or B.

2. The compound of claim 1, wherein the compound is of Formula I-A: 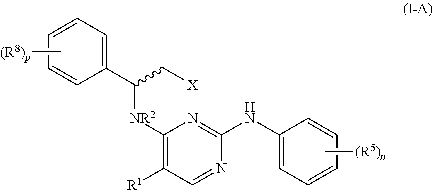
or a pharmaceutically acceptable salt, solvate, enantiomer, stereoisomer, or a tautomer thereof, wherein p is an integer selected from 0, 1, 2, 3, 4, 5, and all other variables are as defined herein.

3. The compound of claim 2, wherein the compound is of Formula I-A1: 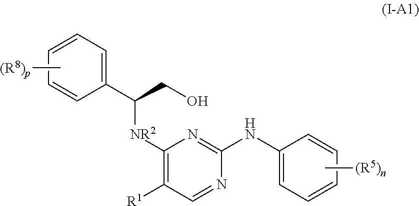
or a pharmaceutically acceptable salt, solvate, enantiomer, stereoisomer, or a tautomer thereof, wherein p is an integer selected from 0, 1, 2, 3, 4, 5, and all other variables are as defined herein.

4. The compound of claim 1, wherein the compound is of Formula I-B1, I-B2, I-B3, I-B3′, or I-B4: 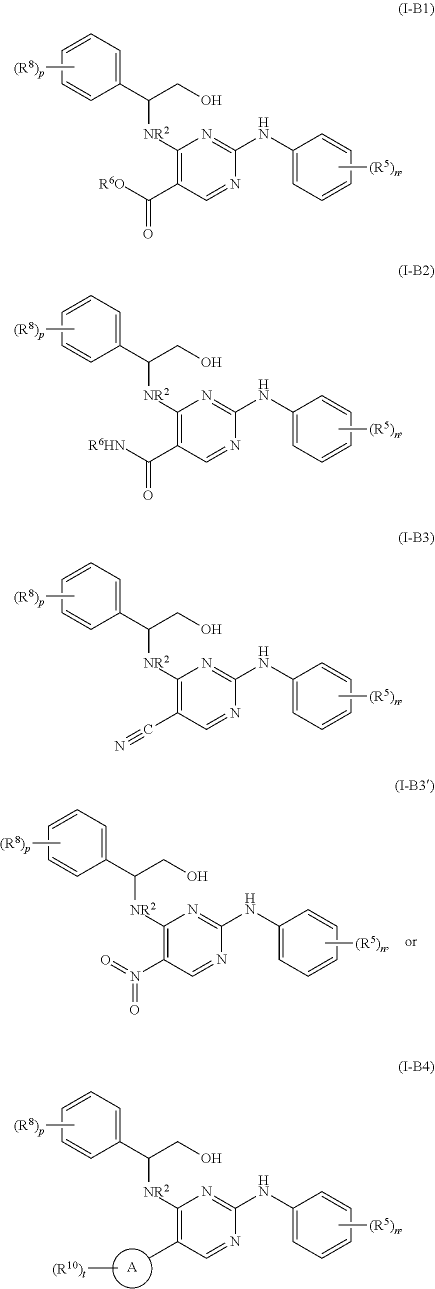
wherein Ring A represents a monocyclic heteroaryl comprising one or more heteroatoms independently selected from N, S, and O; R¹⁰ is independently —OH, oxo, halogen, C₁₋₄ alkoxy, C₁₋₆ alkyl, 4-7 membered monocyclic heterocycloalkyl; t is an integer selected from 0, 1, 2, 3, 4; and p is an integer selected from 0, 1, 2, 3, 4 and 5, and all other variables are as defined herein, or a pharmaceutically acceptable salt, solvate, enantiomer, stereoisomer, or a tautomer thereof.

5. The compound of claim 1, wherein the compound is of Formula I-C: 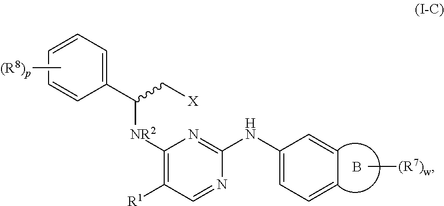
or pharmaceutically acceptable salts, solvates, enantiomers, stereoisomers, and tautomers thereof, wherein Ring B is 6-7 membered heterocyclyl containing at least one heteroatom selected from N, O, S, wherein w is an integer selected from 0, 1, 2 and 3, and all other variables are as defined herein.

6. The compound of claim 1, wherein the compound is of Formula I-D: 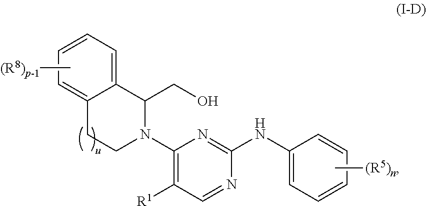
or pharmaceutically acceptable salts, solvates, enantiomers, stereoisomers, and tautomers thereof, wherein u is an integer selected from 0 or 1, and p is an integer from 1, 2, 3, 4 and 5 and all other variables are as defined herein.

7. The compound of claim 1, wherein the compound is of Formula I-E: 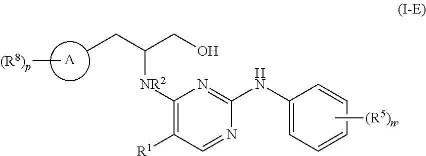
or pharmaceutically acceptable salts, solvates, prodrugs, enantiomers, stereoisomers, and tautomers thereof, wherein Ring D is aryl or heteroaryl; p is an integer from 0, 1, 2, 3, 4 and 5 and all other variables are as defined herein.

8. The compound of claim 7, wherein the compound is of Formula I-F: 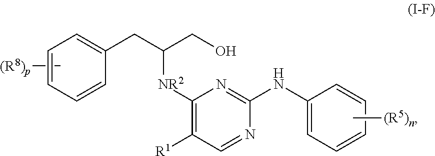
or pharmaceutically acceptable salts, solvates, enantiomers, stereoisomers, and tautomers thereof, wherein p is an integer selected from 0, 1, 2, 3, 4 and 5 and all other variables are as defined herein.

9. The compound of claim 1, wherein the compound is of Formula I-G 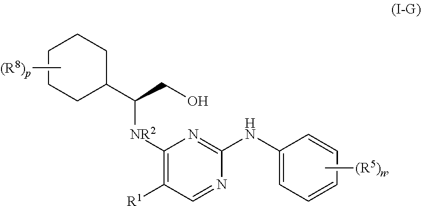
or a pharmaceutically acceptable salt, solvate, enantiomer, stereoisomer, or a tautomer thereof, wherein p is an integer selected from 0, 1, 2, 3, 4 and 5.

10. The compound of claim 1, wherein the compound is of Formula I-H: 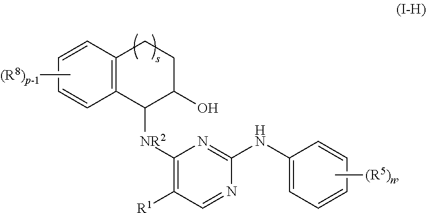
or a pharmaceutically acceptable salt, solvate, enantiomer, stereoisomer, or a tautomer thereof, wherein n is an integer selected from 0, 1, 2, 3, 4, and 5; p is an integer selected from 1, 2, 3, 4 and 5; and s is 0 or 1.

11. The compound of claim 1, wherein R¹ is selected from the groups below:
—CN
—NO₂
—C(O)OH
—C(O)OC₂H₅
—C(O)NH₂
—C(O)NHCH₃
—C(O)NHOH
—C(O)N(CH₃)₂
—C(O)NHCH₂CH₃
—C(O)NHCH(CH₃)₂
—C(O)OCH₂CH₂OH
—C(O)OCH₂CH₂OCH₃ 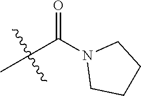 [SMILES: *C(=O)N1CCCC1] 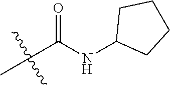 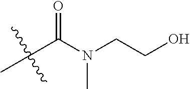 [SMILES: *C(=O)N(C)CCO] 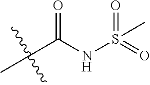 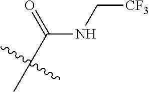 [SMILES: *C(=O)NCC(F)(F)F]
—S(O)₂C₁₋₆ alkyl

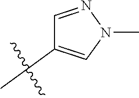 [SMILES: *c1cnn(C)c1]

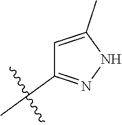 [SMILES: *c1cc(C)[nH]n1]

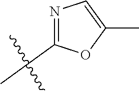 [SMILES: *c1ncc(C)o1]

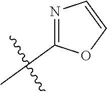 [SMILES: *c1ncco1]

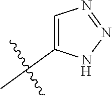

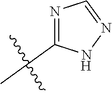

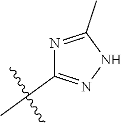 [SMILES: *c1n[nH]c(C)n1]

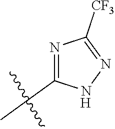

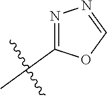 [SMILES: *c1nnco1]

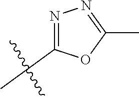 [SMILES: *c1nnc(C)o1]

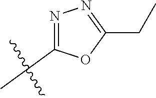 [SMILES: *c1nnc(CC)o1]

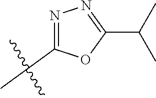 [SMILES: *c1nnc(C(C)C)o1]

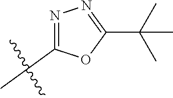 [SMILES: *c1nnc(C(C)(C)C)o1]

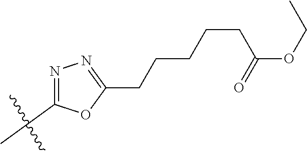 [SMILES: *c1nnc(CCCCCC(=O)OCC)o1]

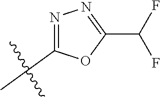 [SMILES: *c1nnc(C(F)F)o1]

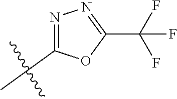 [SMILES: *c1nnc(C(F)(F)F)o1]

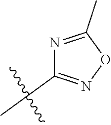 [SMILES: *c1noc(C)n1]

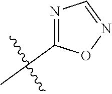 [SMILES: *c1ncno1]

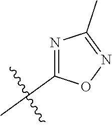 [SMILES: *c1nc(C)no1]

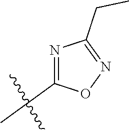 [SMILES: *c1nc(CC)no1]

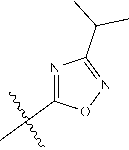 [SMILES: *c1nc(C(C)C)no1]

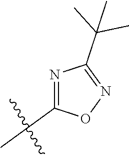 [SMILES: *c1nc(C(C)(C)C)no1]

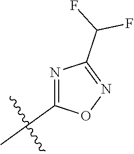 [SMILES: *c1nc(C(F)F)no1]

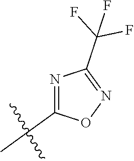 [SMILES: *c1nc(C(F)(F)F)no1]

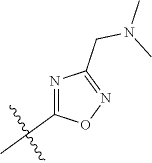 [SMILES: *c1nc(CN(C)C)no1]

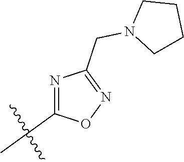 [SMILES: *c1nc(CN2CCCC2)no1]

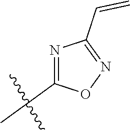 [SMILES: *c1nc(C=C)no1]

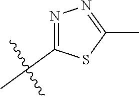 [SMILES: *c1nnc(C)s1]

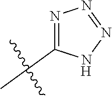

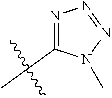 [SMILES: *c1nnnn1C]

12. The compound of claim 1, wherein the group 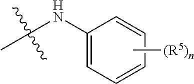
is selected from the groups presented below

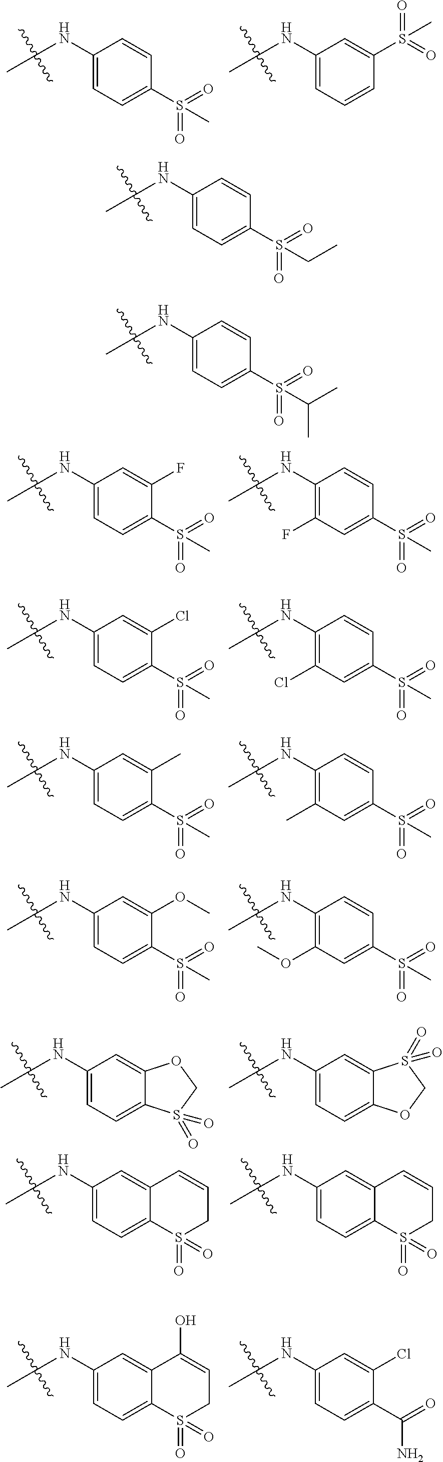

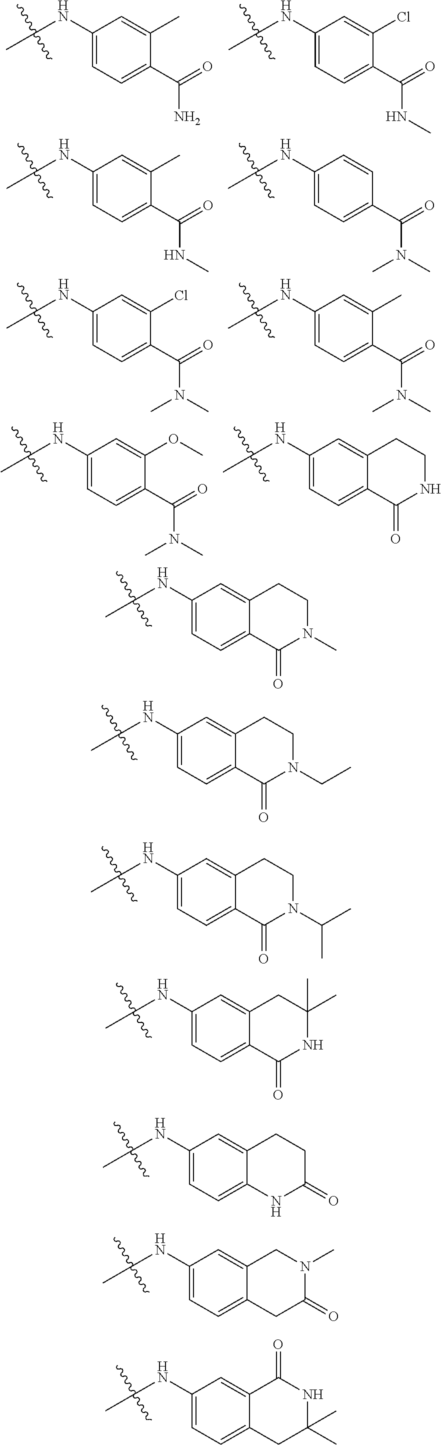

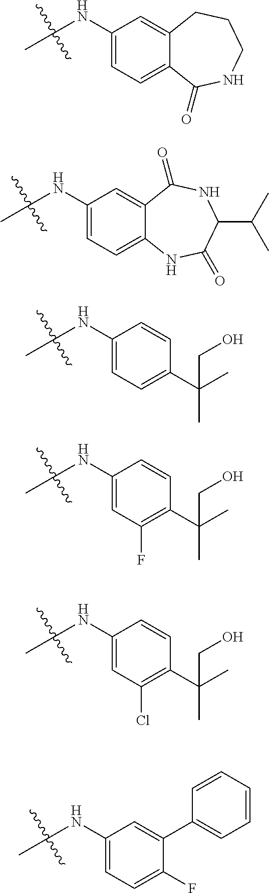

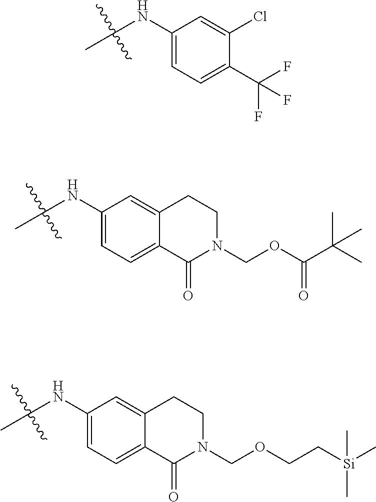 [SMILES: *Nc1ccc(C(F)(F)F)c(Cl)c1 ; *Nc1ccc2c(c1)CCN(COC(=O)C(C)(C)C)C2=O ; *Nc1ccc2c(c1)CCN(COCC[Si](C)(C)C)C2=O]

13. A compound selected from the group consisting of: 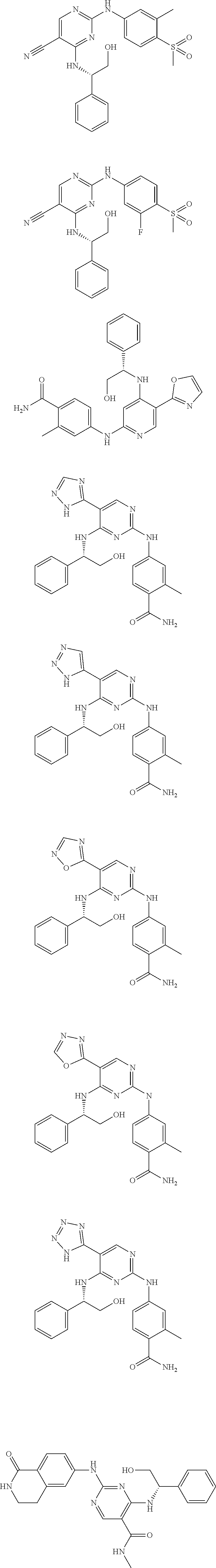 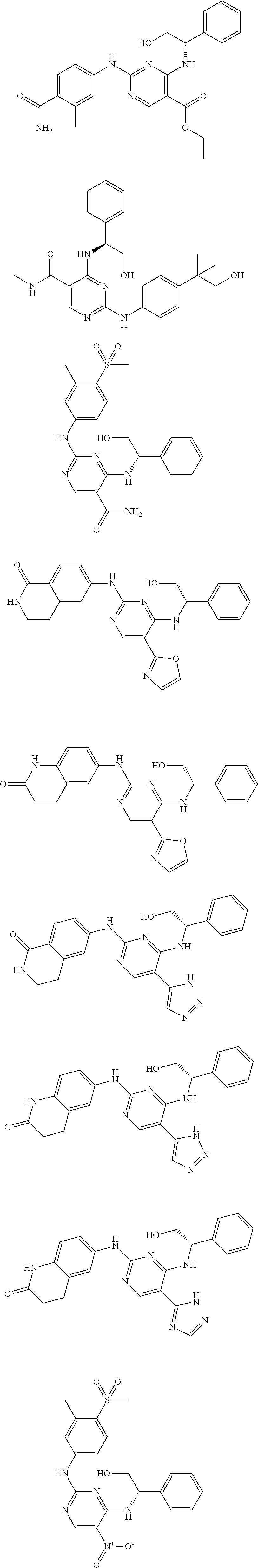 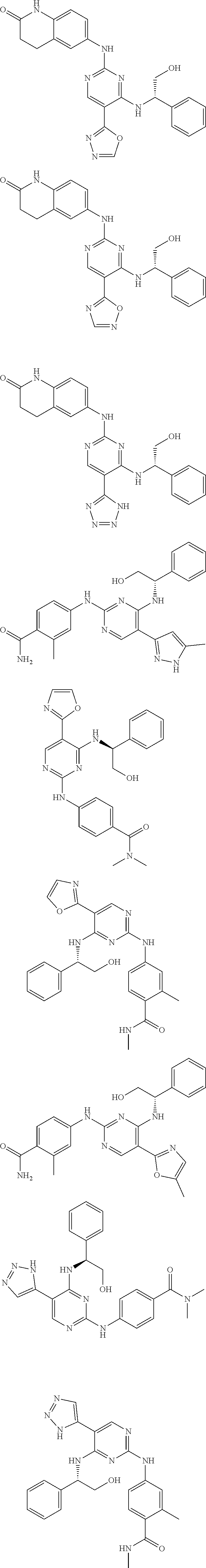 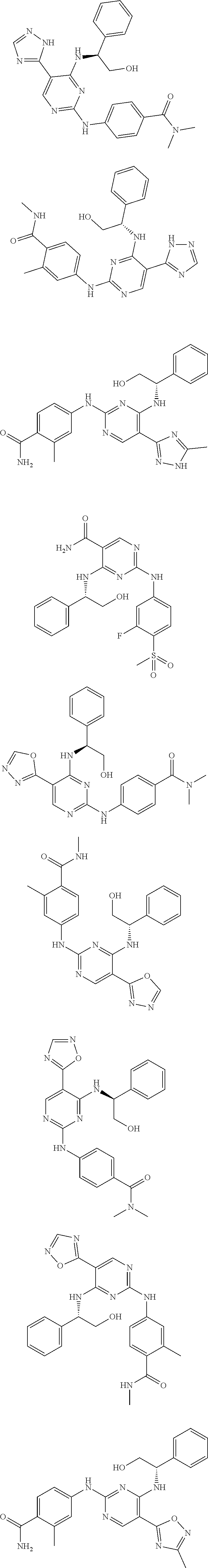 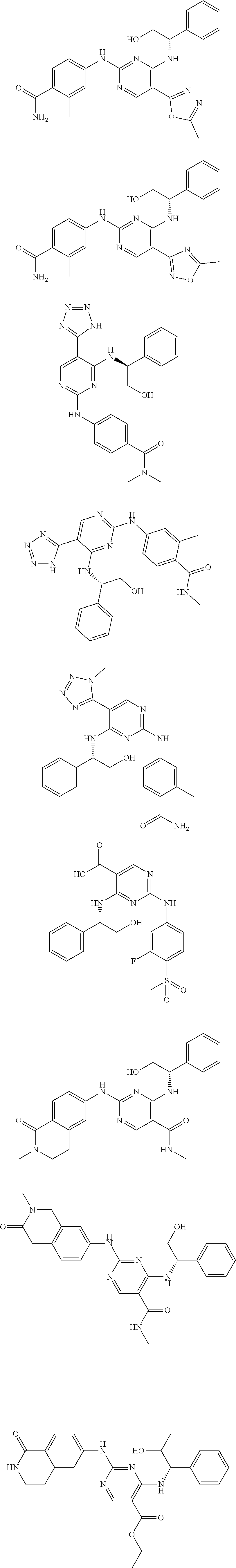 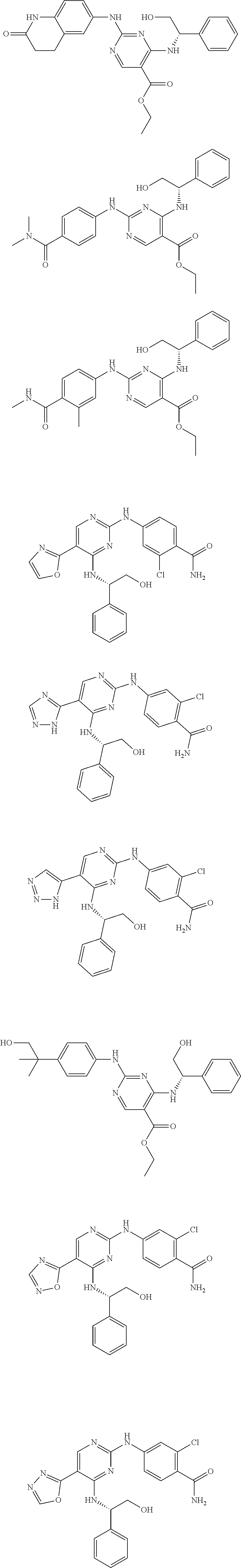 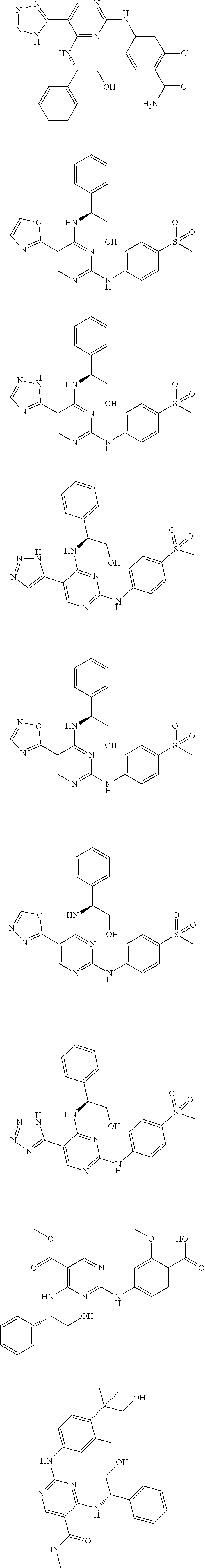 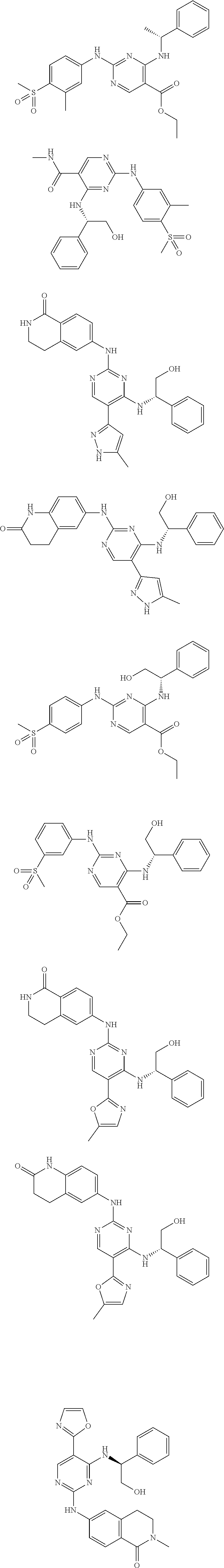 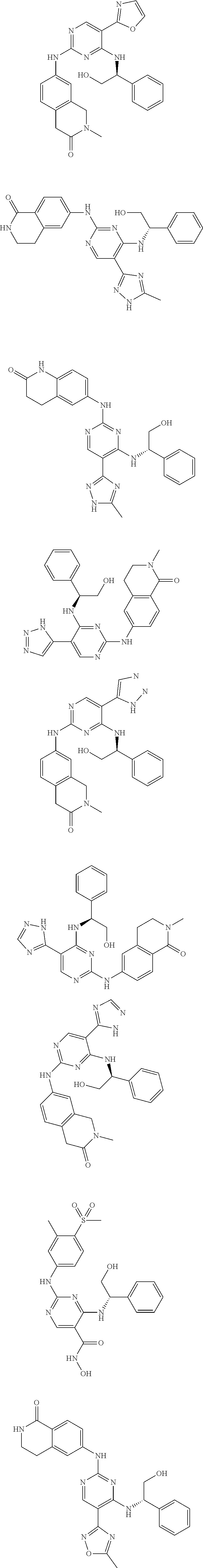 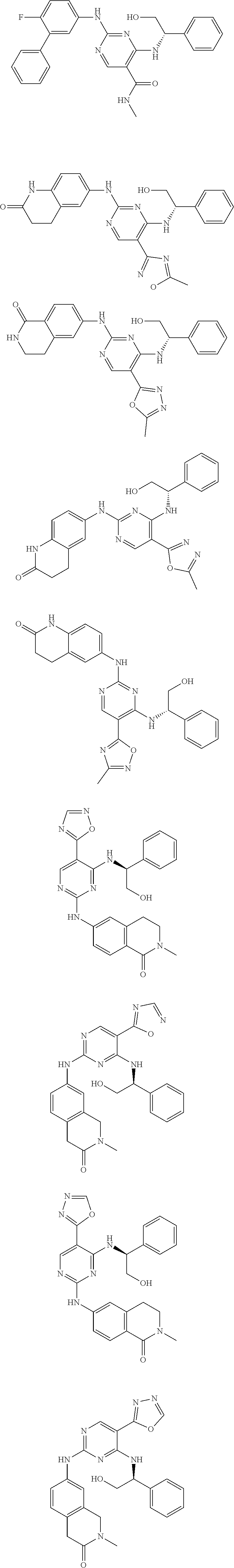 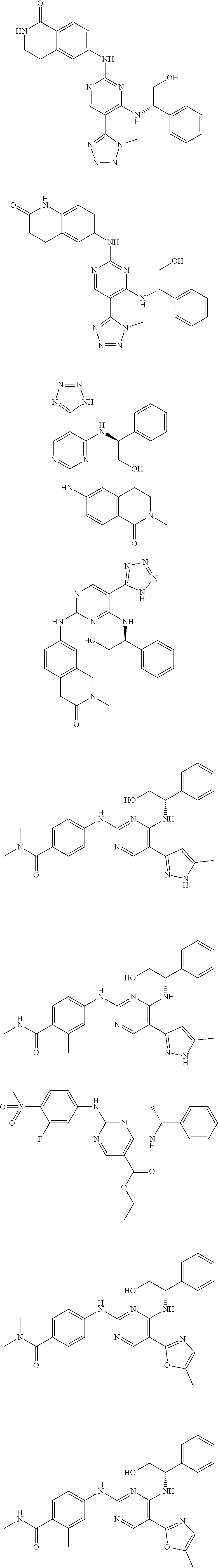 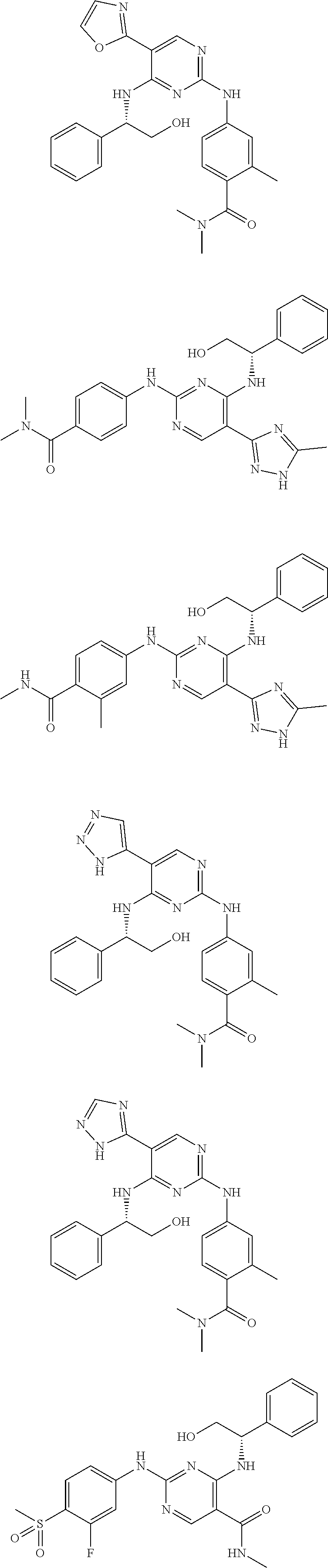 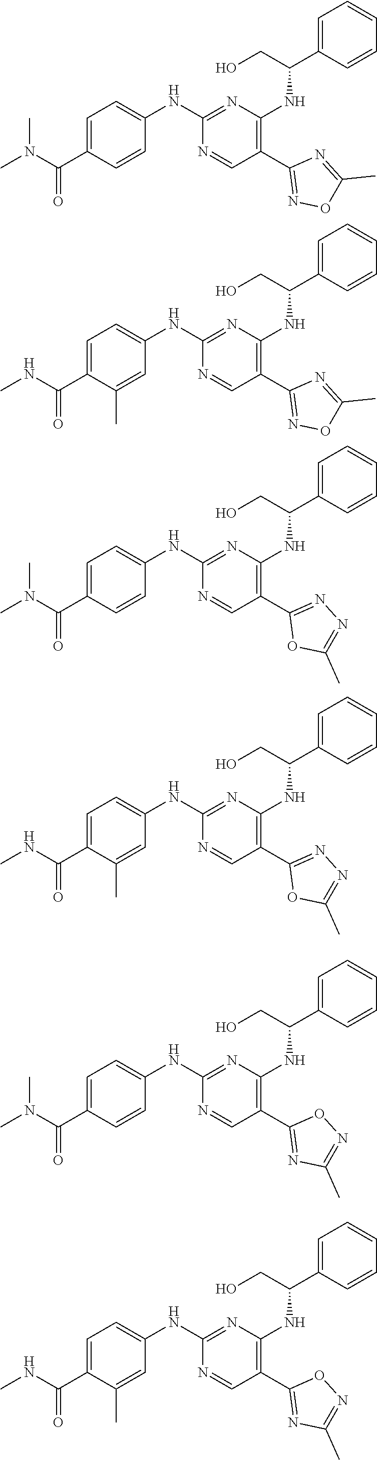 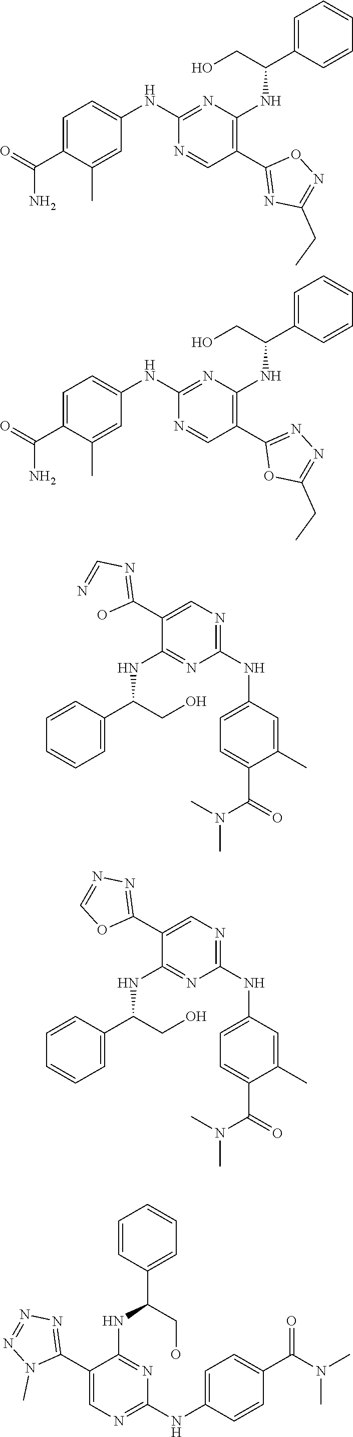 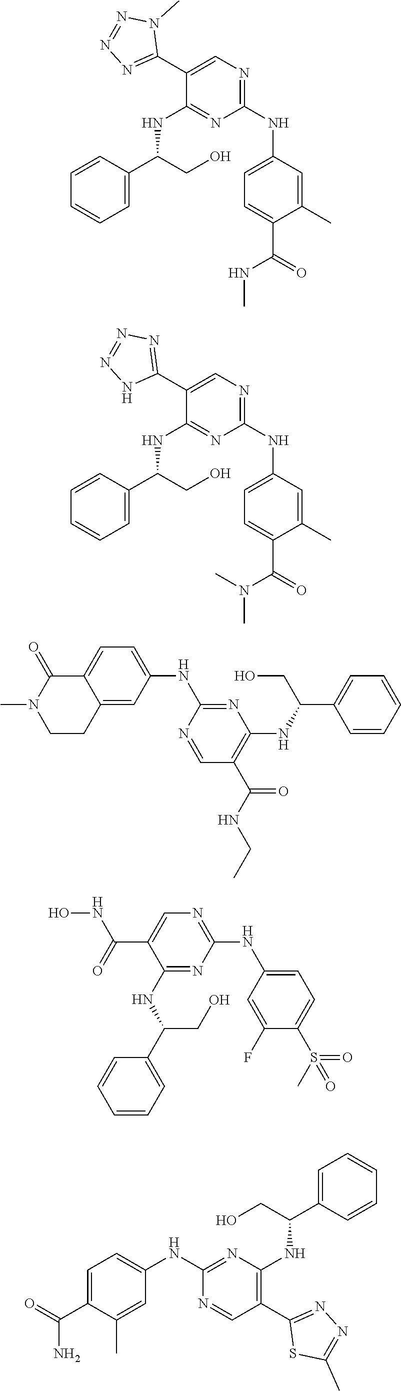 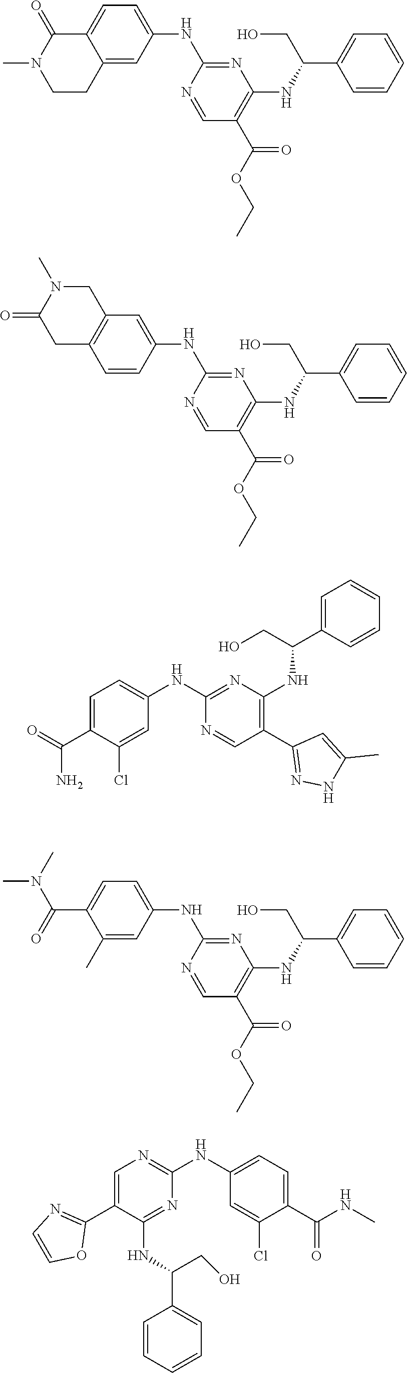 [SMILES: CCOC(=O)c1cnc(Nc2ccc3c(c2)CCN(C)C3=O)nc1N[C@H](CO)c1ccccc1 ; CCOC(=O)c1cnc(Nc2ccc3c(c2)CN(C)C(=O)C3)nc1N[C@H](CO)c1ccccc1 ; Cc1cc(-c2cnc(Nc3ccc(C(N)=O)c(Cl)c3)nc2N[C@H](CO)c2ccccc2)n[nH]1 ; CCOC(=O)c1cnc(Nc2ccc(C(=O)N(C)C)c(C)c2)nc1N[C@H](CO)c1ccccc1 ; CNC(=O)c1ccc(Nc2ncc(-c3ncco3)c(N[C@H](CO)c3ccccc3)n2)cc1Cl] 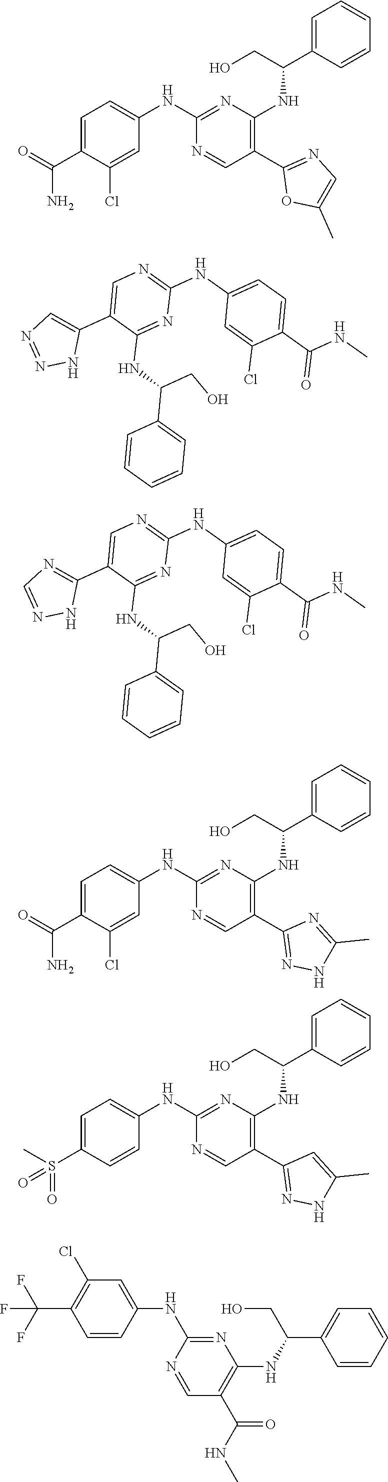 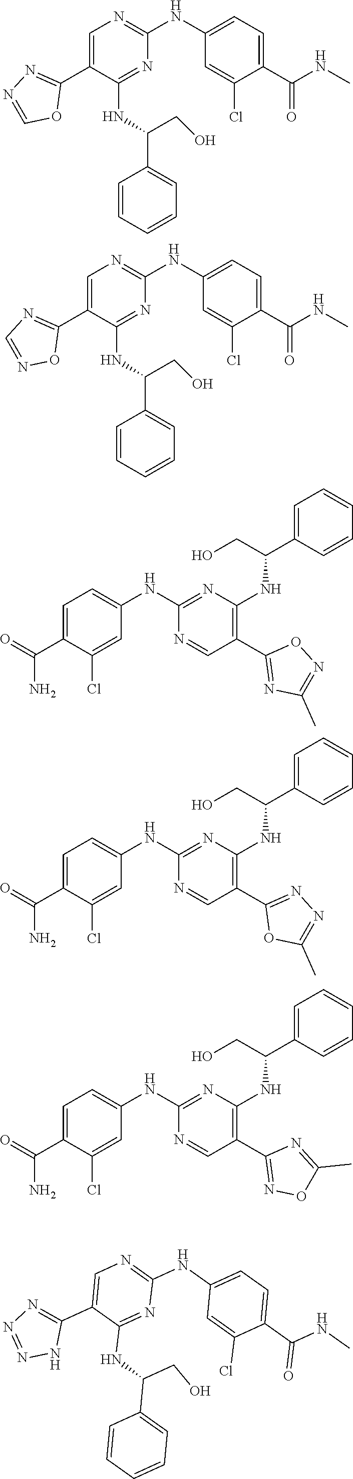 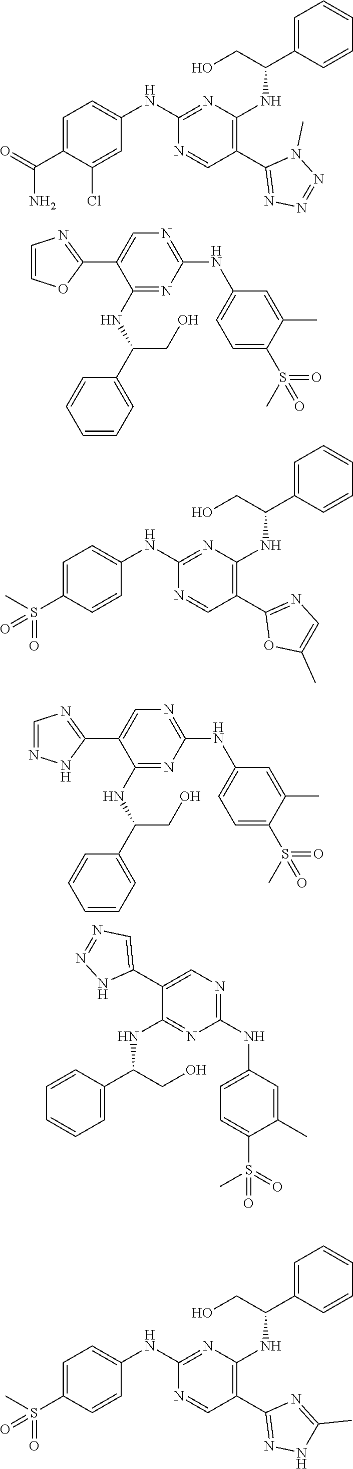 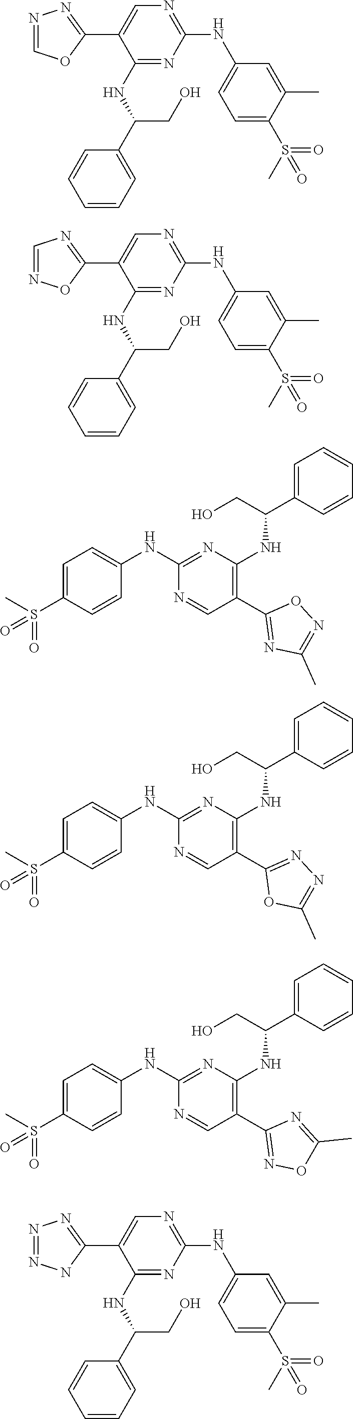 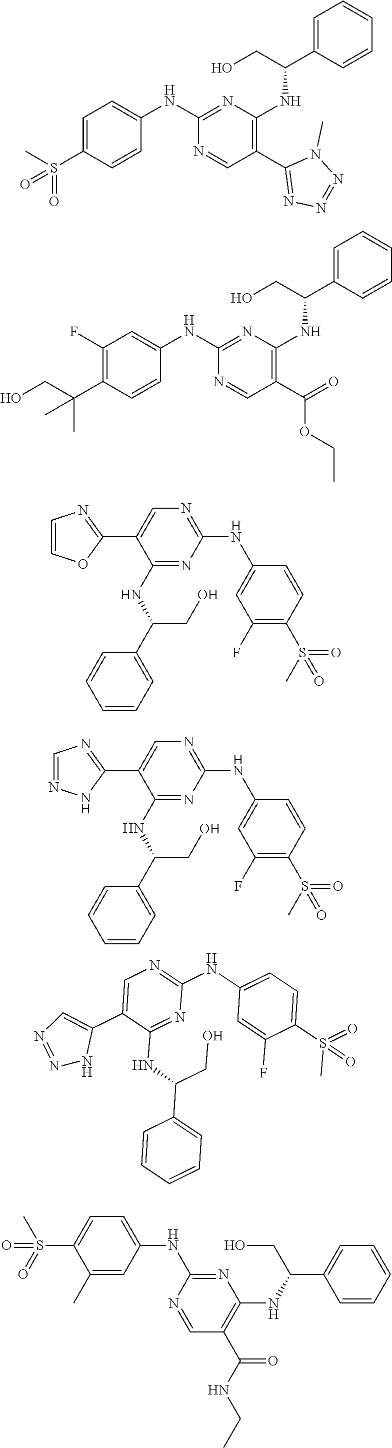 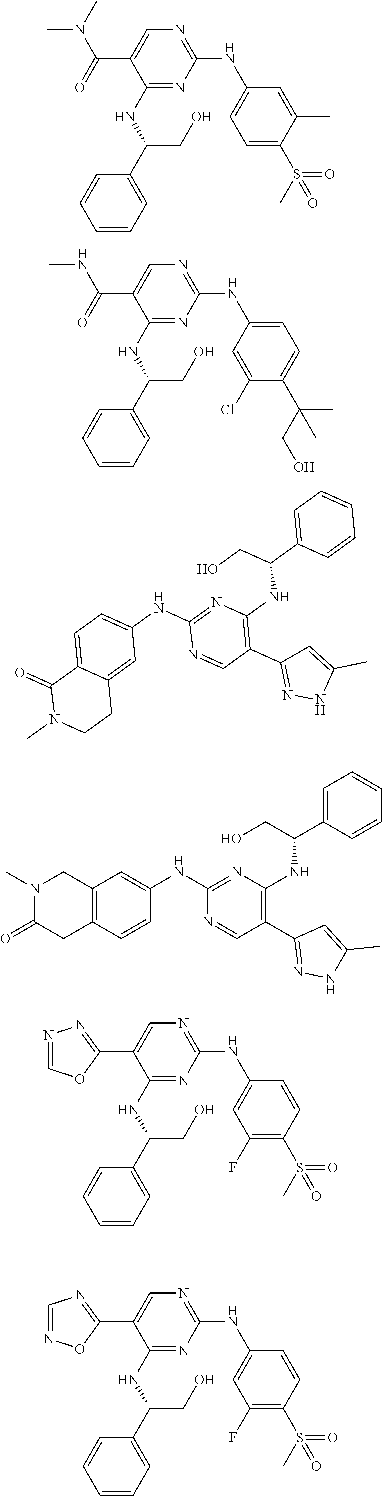 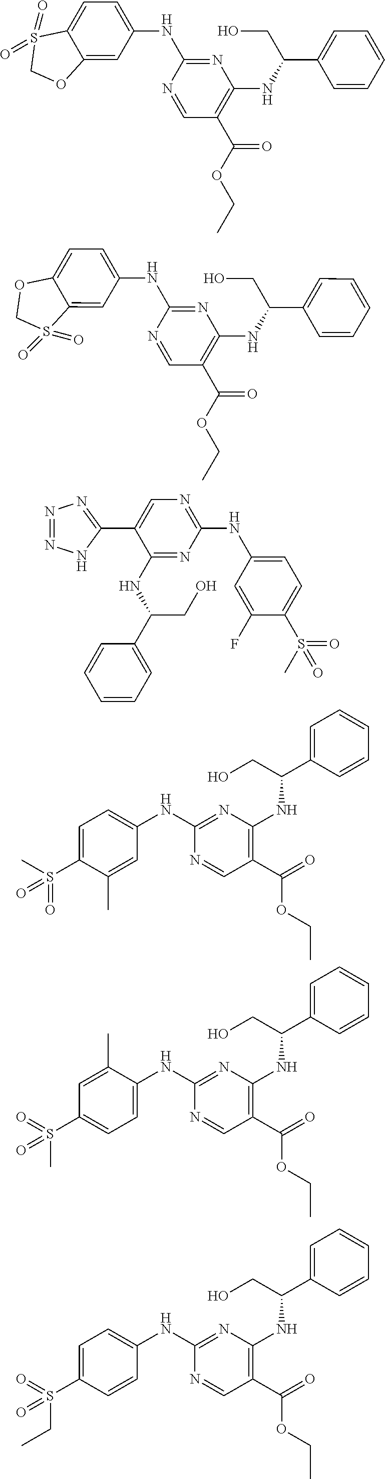 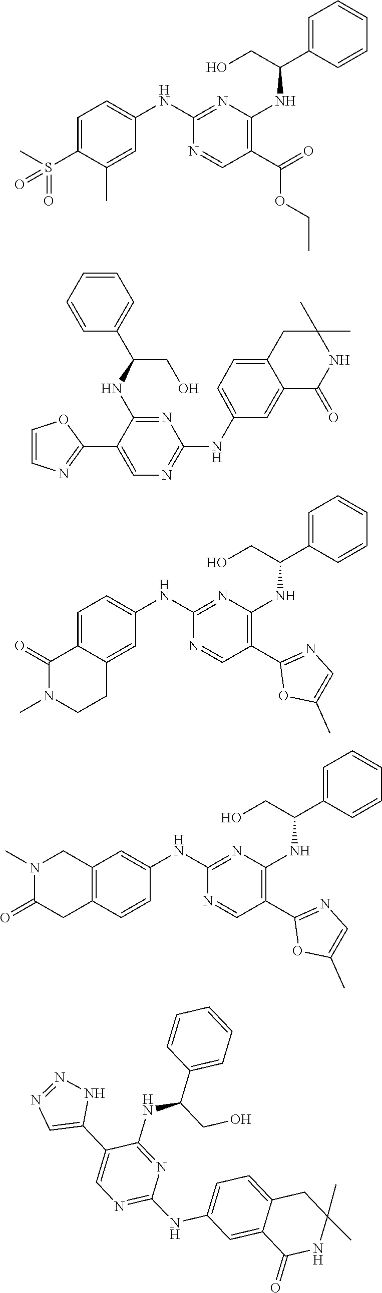 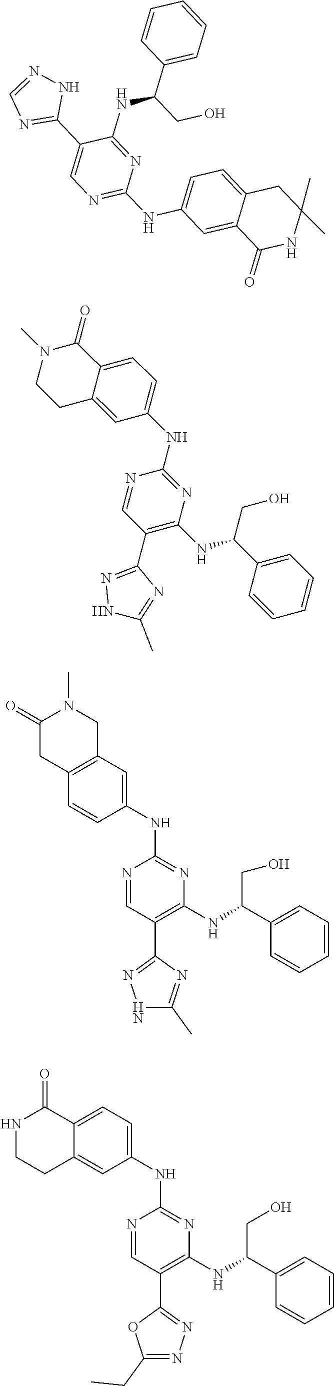 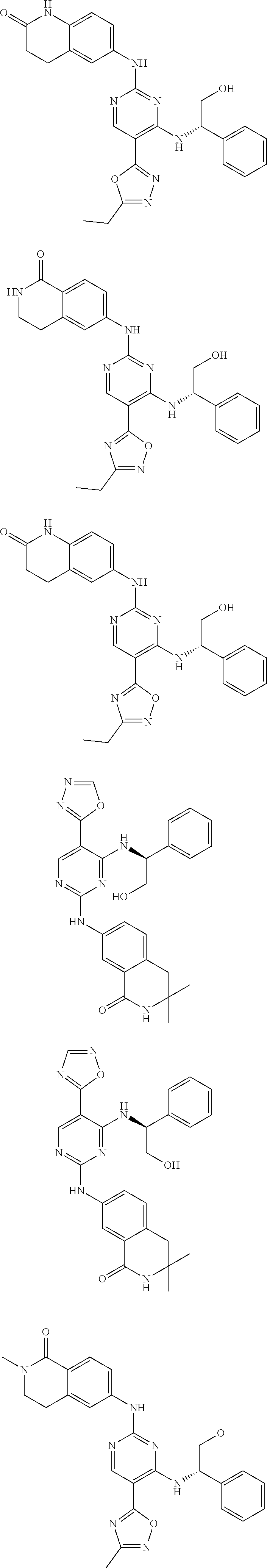 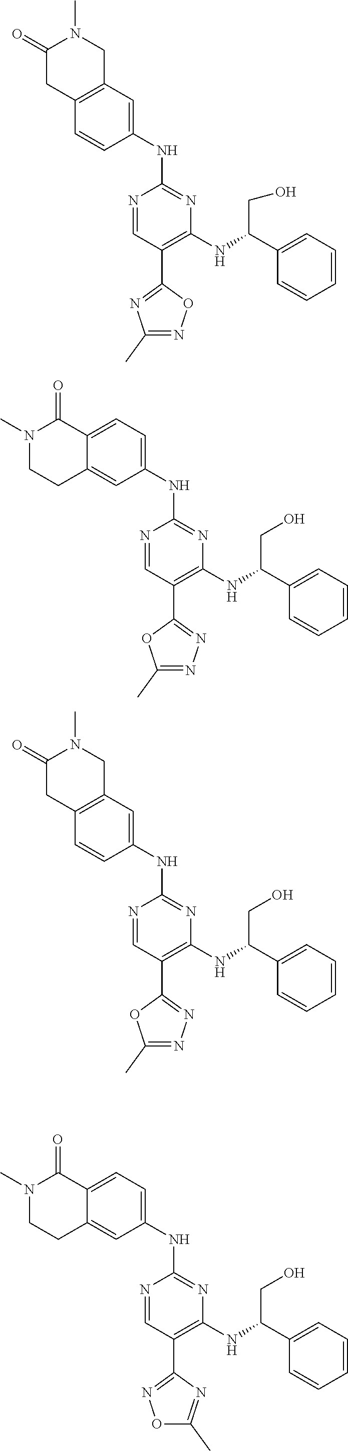 [SMILES: Cc1noc(-c2cnc(Nc3ccc4c(c3)CN(C)C(=O)C4)nc2N[C@H](CO)c2ccccc2)n1 ; Cc1nnc(-c2cnc(Nc3ccc4c(c3)CCN(C)C4=O)nc2N[C@H](CO)c2ccccc2)o1 ; Cc1nnc(-c2cnc(Nc3ccc4c(c3)CN(C)C(=O)C4)nc2N[C@H](CO)c2ccccc2)o1 ; Cc1nc(-c2cnc(Nc3ccc4c(c3)CCN(C)C4=O)nc2N[C@H](CO)c2ccccc2)no1] 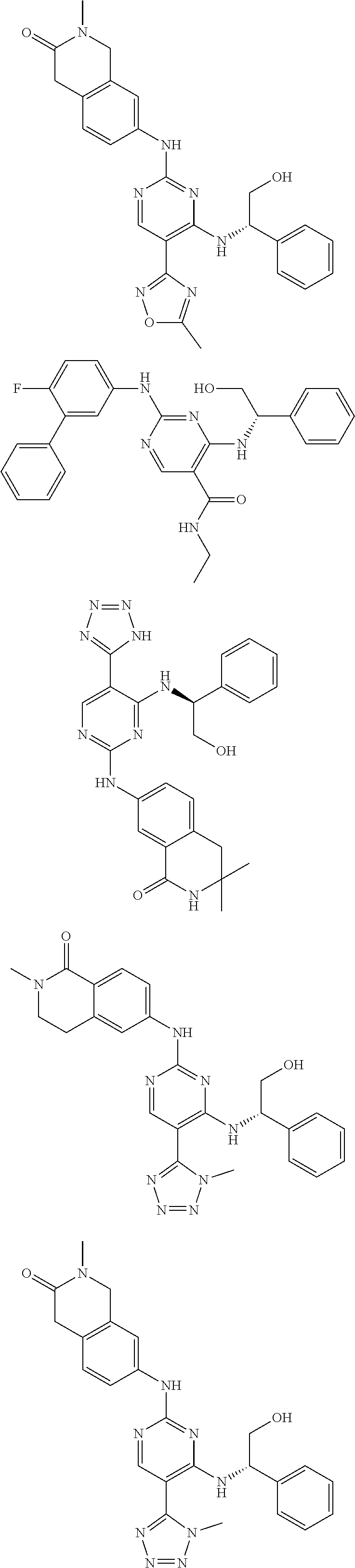 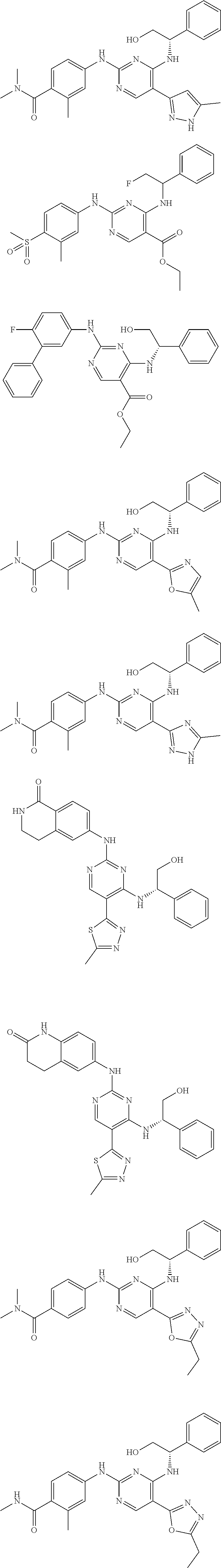 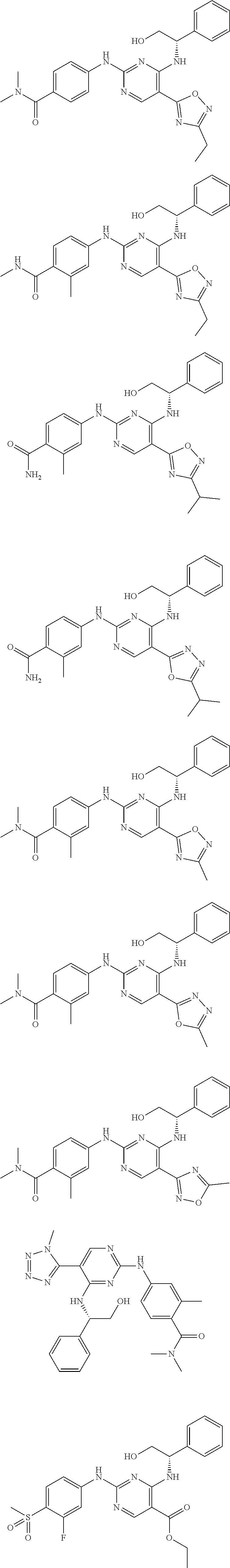 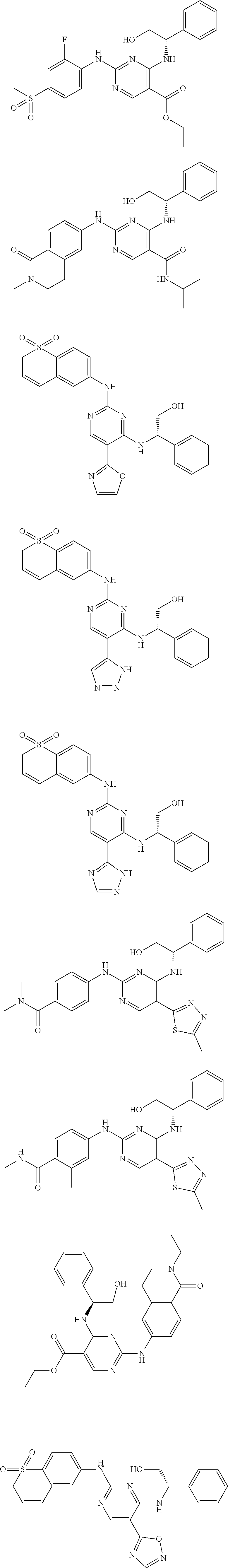 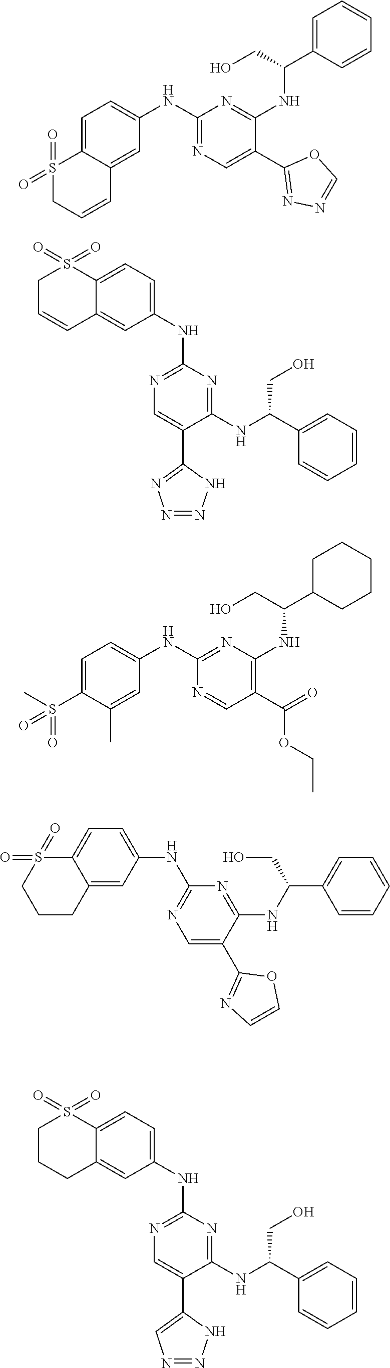 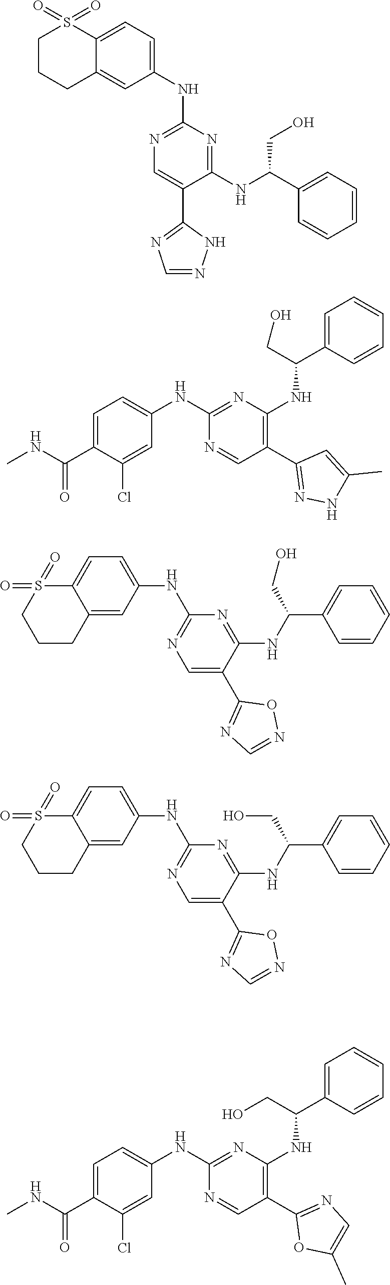 [SMILES: O=S1(=O)CCCc2cc(Nc3ncc(-c4ncn[nH]4)c(N[C@H](CO)c4ccccc4)n3)ccc21 ; CNC(=O)c1ccc(Nc2ncc(-c3cc(C)[nH]n3)c(N[C@H](CO)c3ccccc3)n2)cc1Cl ; O=S1(=O)CCCc2cc(Nc3ncc(-c4ncno4)c(N[C@H](CO)c4ccccc4)n3)ccc21 ; O=S1(=O)CCCc2cc(Nc3ncc(-c4ncno4)c(N[C@H](CO)c4ccccc4)n3)ccc21 ; CNC(=O)c1ccc(Nc2ncc(-c3ncc(C)o3)c(N[C@H](CO)c3ccccc3)n2)cc1Cl] 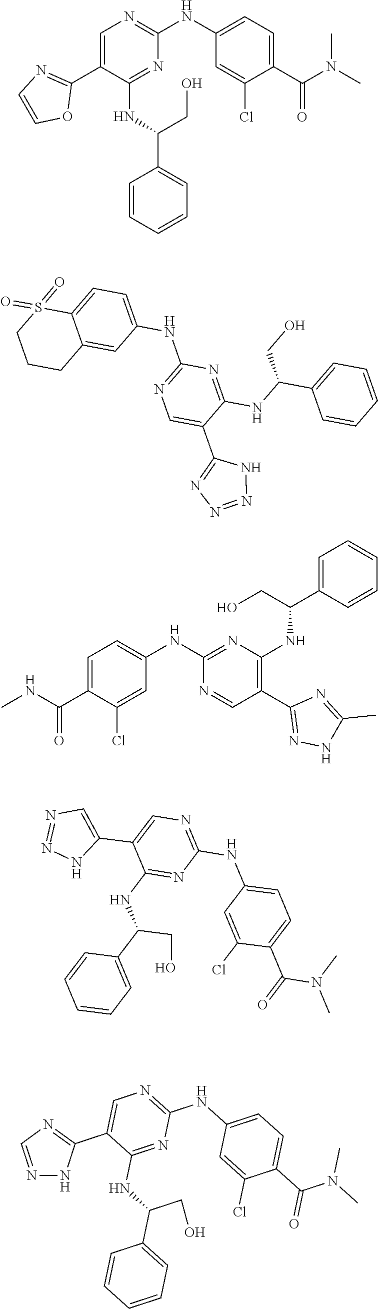 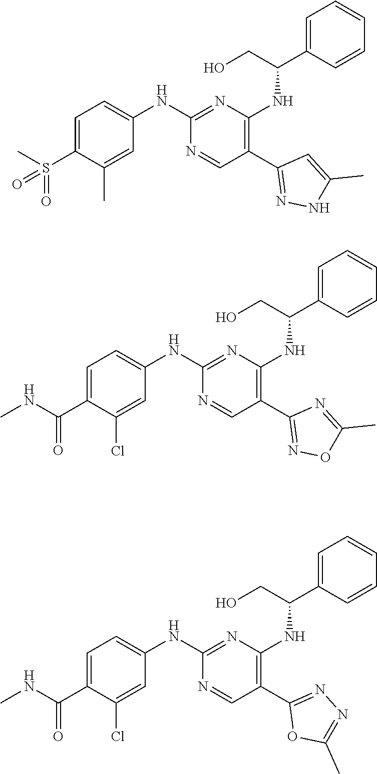 [SMILES: Cc1cc(-c2cnc(Nc3ccc(S(C)(=O)=O)c(C)c3)nc2N[C@H](CO)c2ccccc2)n[nH]1 ; CNC(=O)c1ccc(Nc2ncc(-c3noc(C)n3)c(N[C@H](CO)c3ccccc3)n2)cc1Cl ; CNC(=O)c1ccc(Nc2ncc(-c3nnc(C)o3)c(N[C@H](CO)c3ccccc3)n2)cc1Cl] 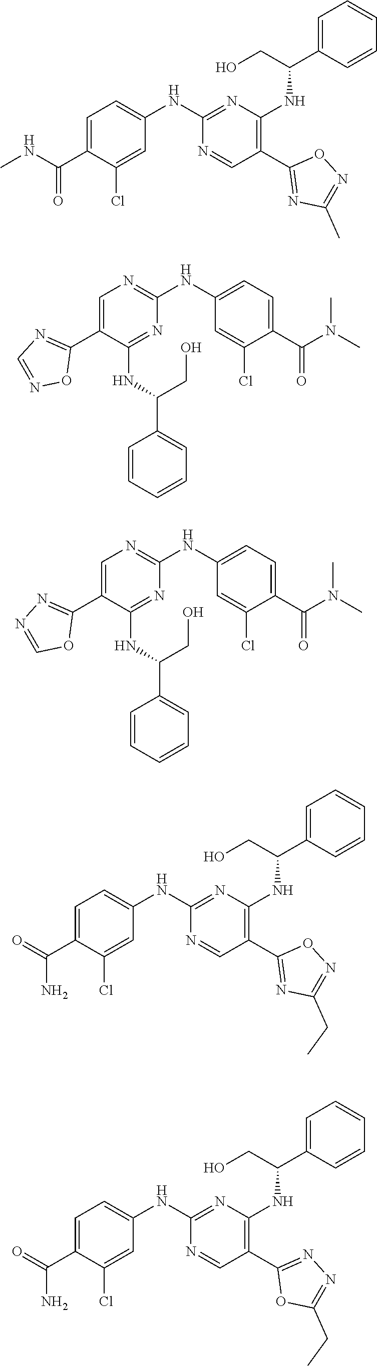 [SMILES: CNC(=O)c1ccc(Nc2ncc(-c3nc(C)no3)c(N[C@H](CO)c3ccccc3)n2)cc1Cl ; CN(C)C(=O)c1ccc(Nc2ncc(-c3ncno3)c(N[C@H](CO)c3ccccc3)n2)cc1Cl ; CN(C)C(=O)c1ccc(Nc2ncc(-c3nnco3)c(N[C@H](CO)c3ccccc3)n2)cc1Cl ; CCc1noc(-c2cnc(Nc3ccc(C(N)=O)c(Cl)c3)nc2N[C@H](CO)c2ccccc2)n1 ; CCc1nnc(-c2cnc(Nc3ccc(C(N)=O)c(Cl)c3)nc2N[C@H](CO)c2ccccc2)o1] 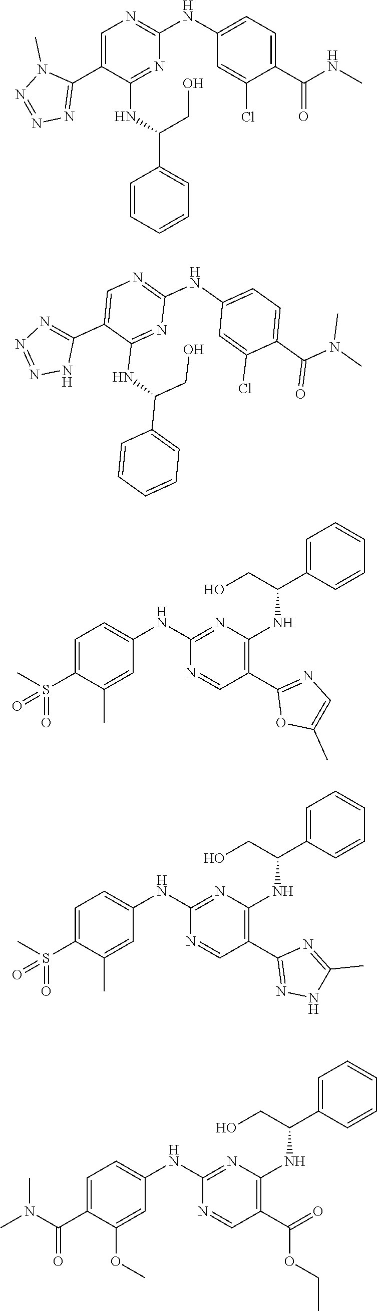 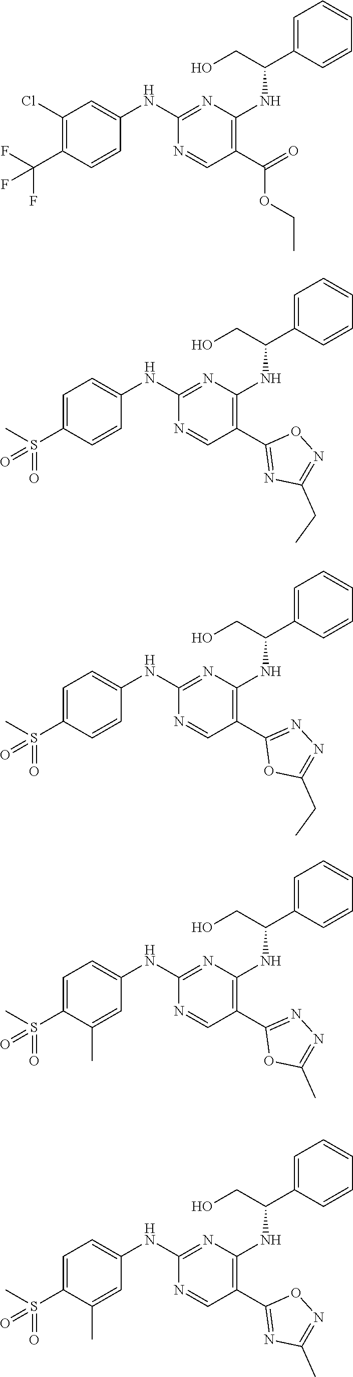 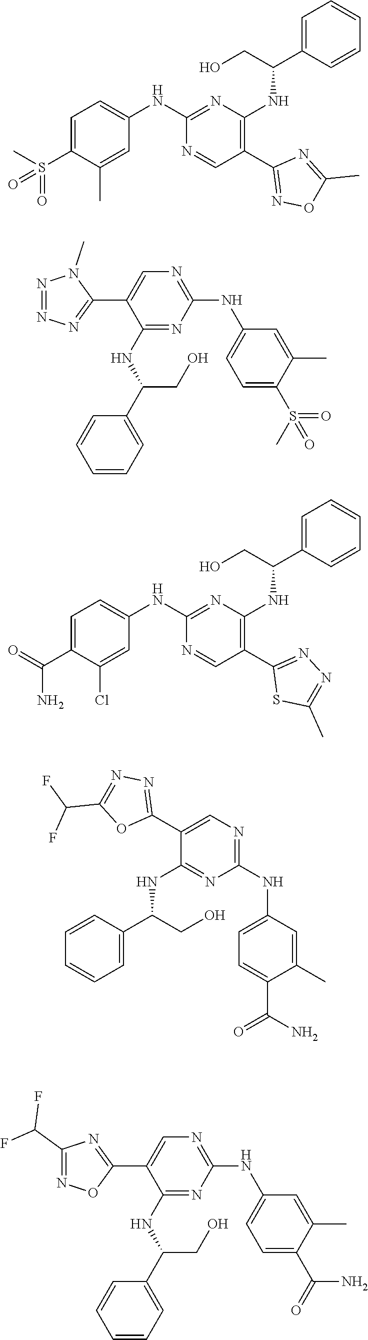 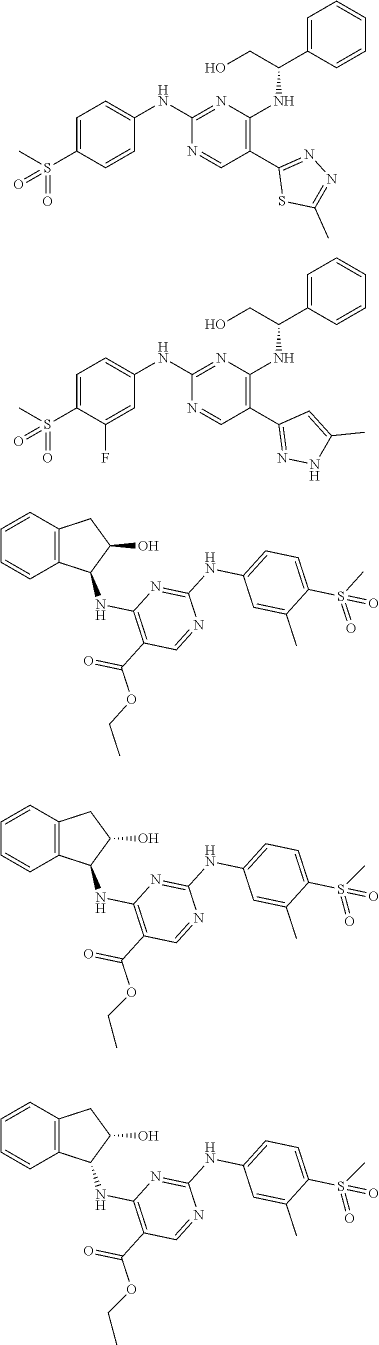 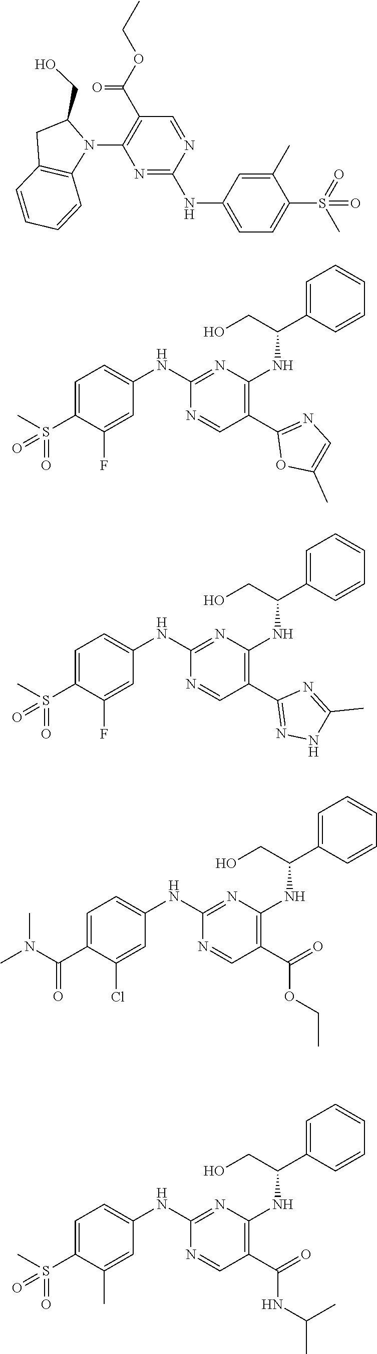 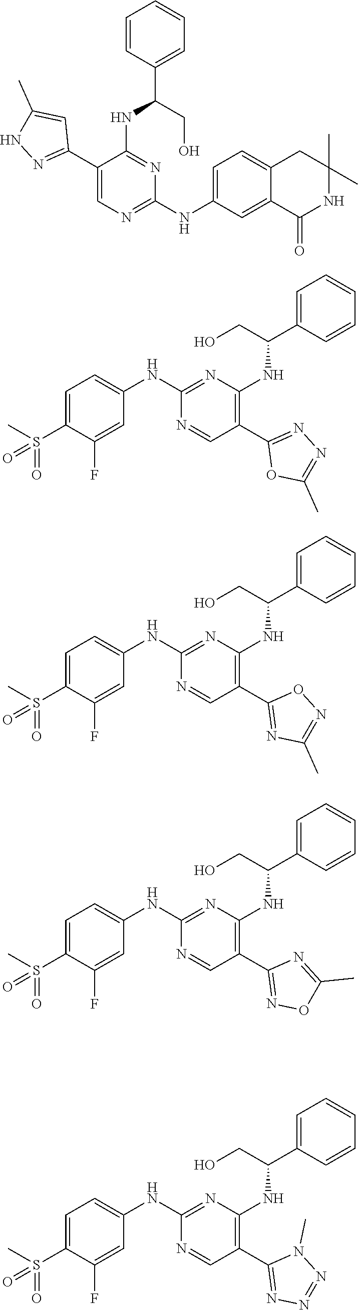 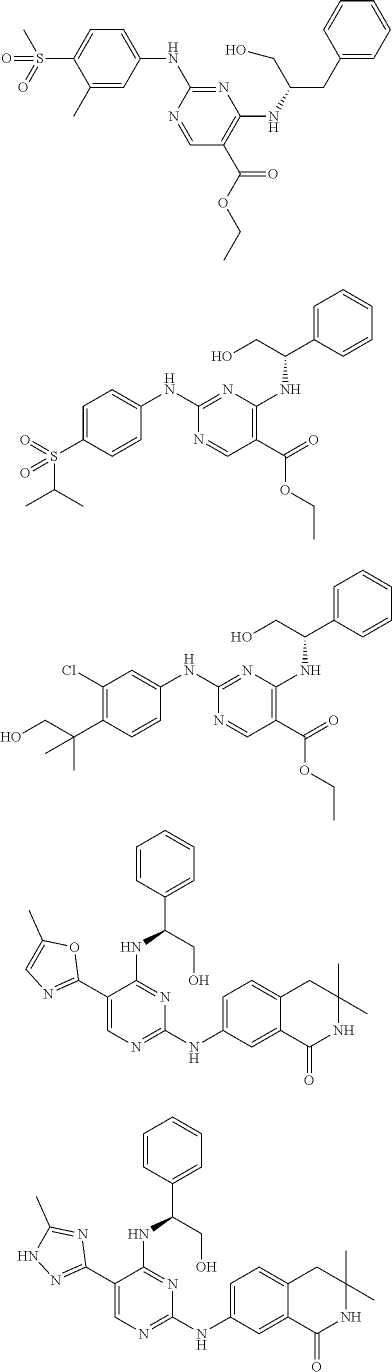 [SMILES: CCOC(=O)c1cnc(Nc2ccc(S(C)(=O)=O)c(C)c2)nc1N[C@H](CO)Cc1ccccc1 ; CCOC(=O)c1cnc(Nc2ccc(S(=O)(=O)C(C)C)cc2)nc1N[C@H](CO)c1ccccc1 ; CCOC(=O)c1cnc(Nc2ccc(C(C)(C)CO)c(Cl)c2)nc1N[C@H](CO)c1ccccc1 ; Cc1cnc(-c2cnc(Nc3ccc4c(c3)C(=O)NC(C)(C)C4)nc2N[C@H](CO)c2ccccc2)o1 ; Cc1nc(-c2cnc(Nc3ccc4c(c3)C(=O)NC(C)(C)C4)nc2N[C@H](CO)c2ccccc2)n[nH]1] 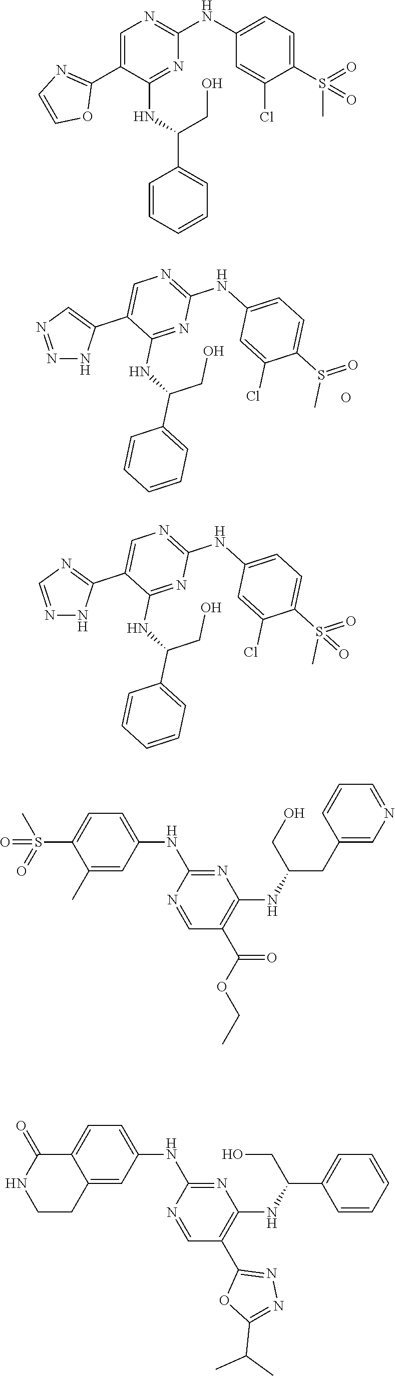 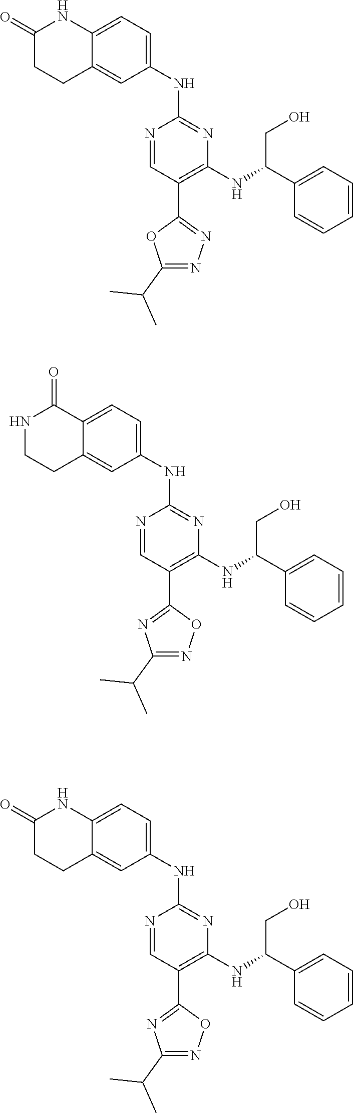 [SMILES: CC(C)c1nnc(-c2cnc(Nc3ccc4c(c3)CCC(=O)N4)nc2N[C@H](CO)c2ccccc2)o1 ; CC(C)c1noc(-c2cnc(Nc3ccc4c(c3)CCNC4=O)nc2N[C@H](CO)c2ccccc2)n1 ; CC(C)c1noc(-c2cnc(Nc3ccc4c(c3)CCC(=O)N4)nc2N[C@H](CO)c2ccccc2)n1] 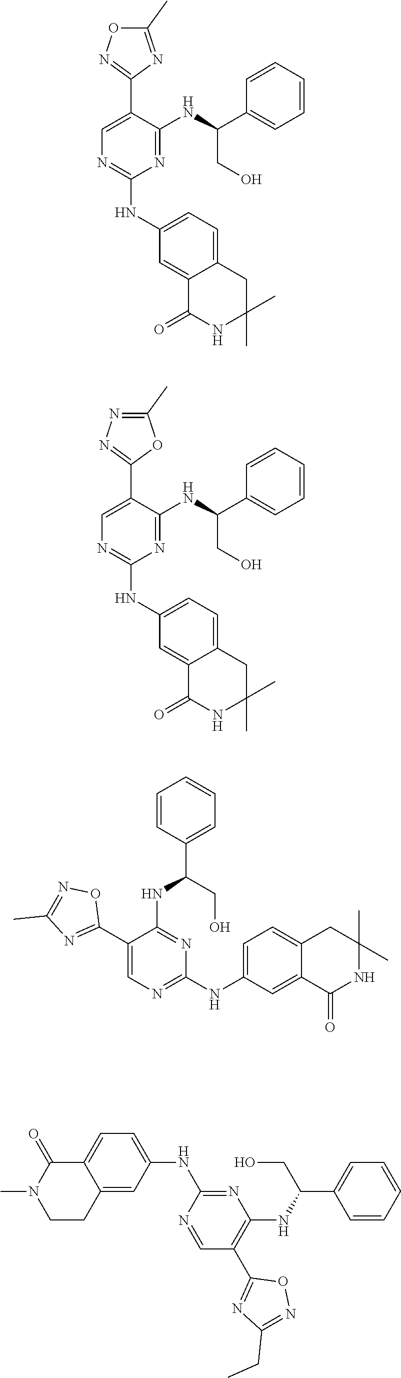 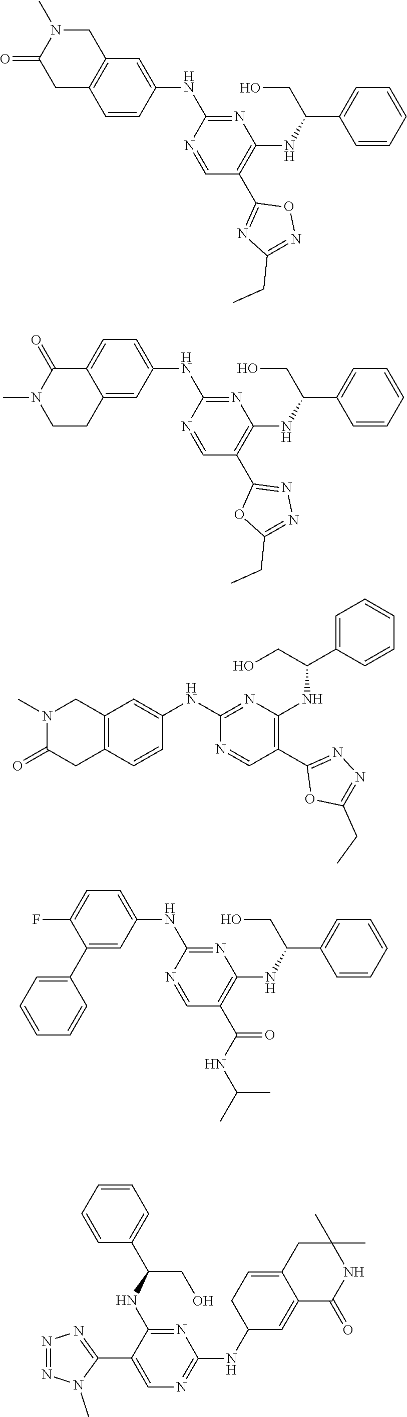 [SMILES: CCc1noc(-c2cnc(Nc3ccc4c(c3)CN(C)C(=O)C4)nc2N[C@H](CO)c2ccccc2)n1 ; CCc1nnc(-c2cnc(Nc3ccc4c(c3)CCN(C)C4=O)nc2N[C@H](CO)c2ccccc2)o1 ; CCc1nnc(-c2cnc(Nc3ccc4c(c3)CN(C)C(=O)C4)nc2N[C@H](CO)c2ccccc2)o1 ; CC(C)NC(=O)c1cnc(Nc2ccc(F)c(-c3ccccc3)c2)nc1N[C@H](CO)c1ccccc1 ; Cn1nnnc1-c1cnc(NC2C=C3C(=O)NC(C)(C)CC3=CC2)nc1N[C@H](CO)c1ccccc1] 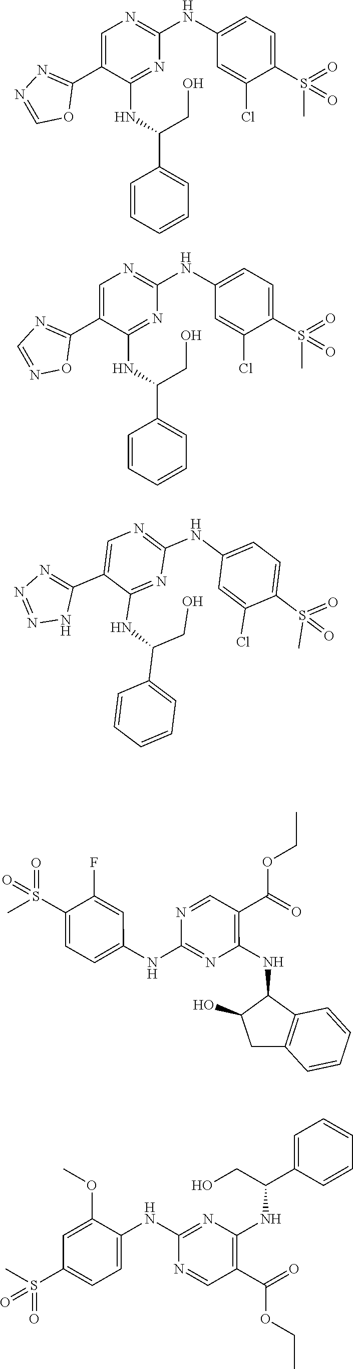 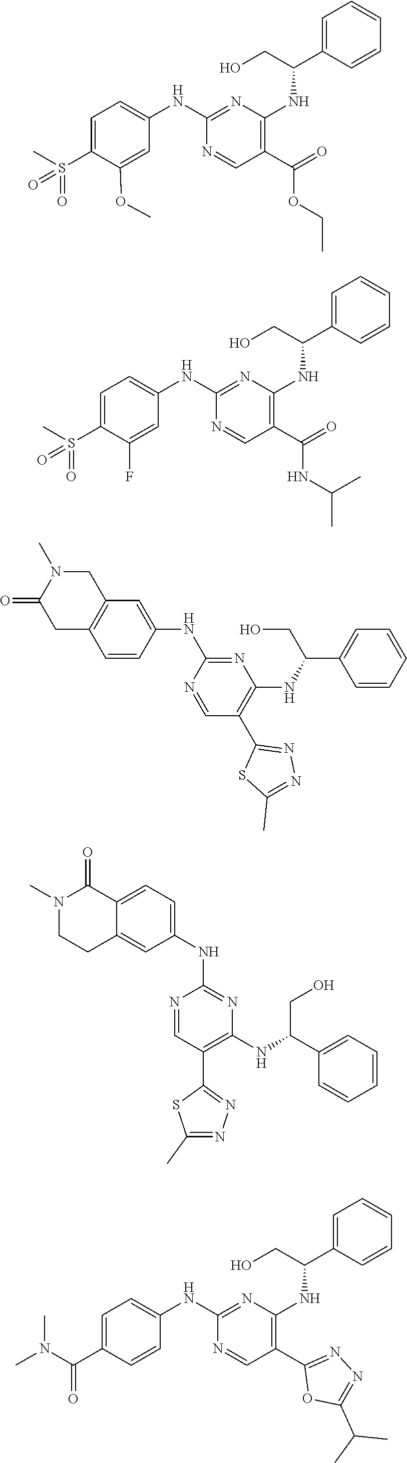 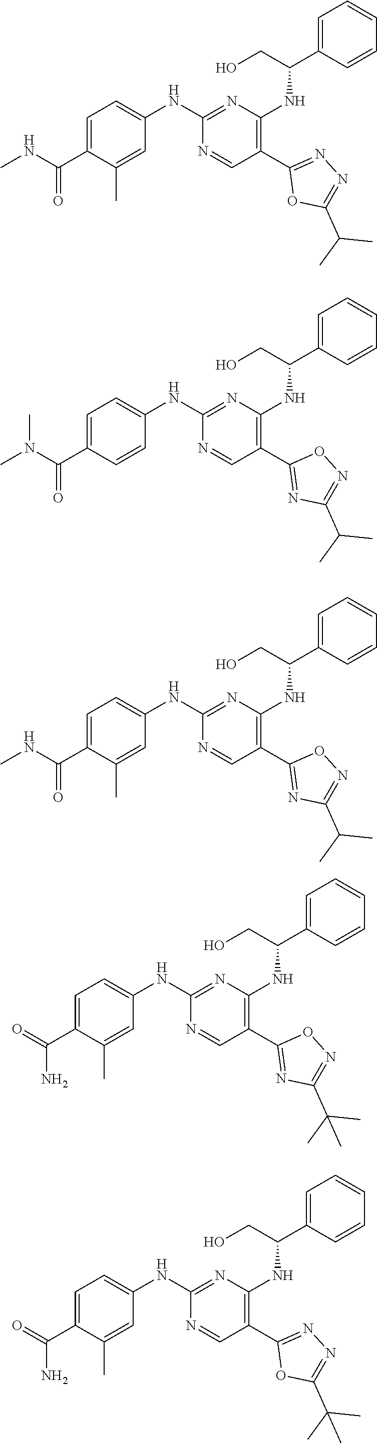 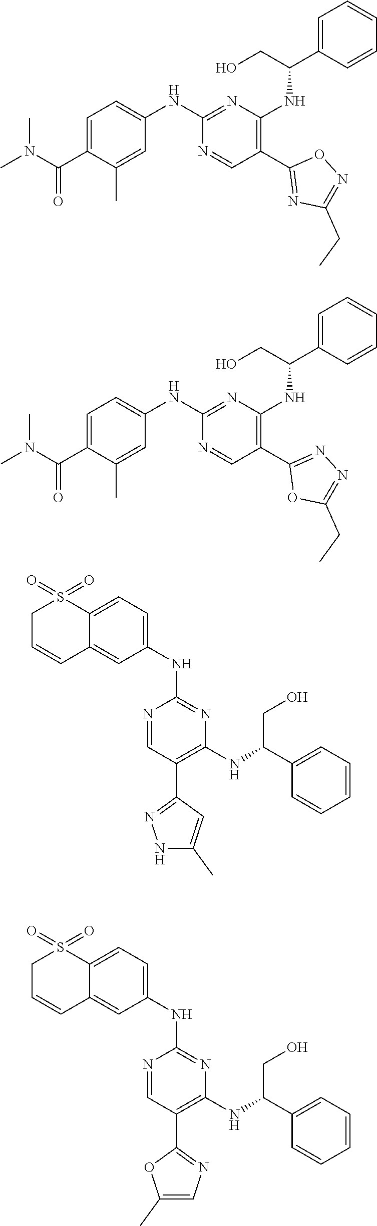 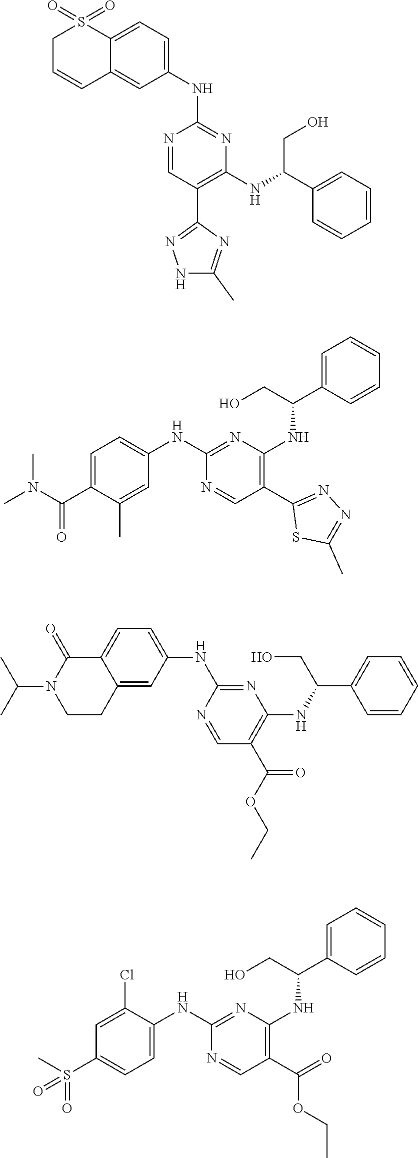 [SMILES: Cc1nc(-c2cnc(Nc3ccc4c(c3)C=CCS4(=O)=O)nc2N[C@H](CO)c2ccccc2)n[nH]1 ; Cc1nnc(-c2cnc(Nc3ccc(C(=O)N(C)C)c(C)c3)nc2N[C@H](CO)c2ccccc2)s1 ; CCOC(=O)c1cnc(Nc2ccc3c(c2)CCN(C(C)C)C3=O)nc1N[C@H](CO)c1ccccc1 ; CCOC(=O)c1cnc(Nc2ccc(S(C)(=O)=O)cc2Cl)nc1N[C@H](CO)c1ccccc1] 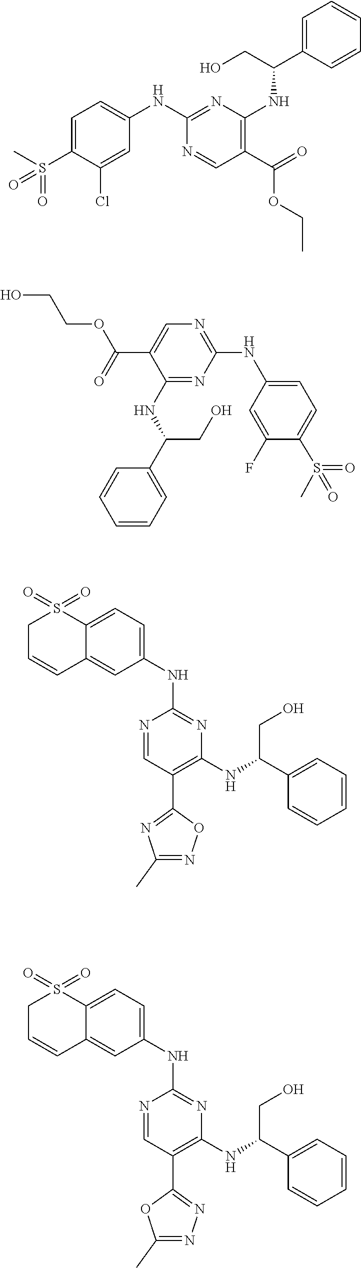 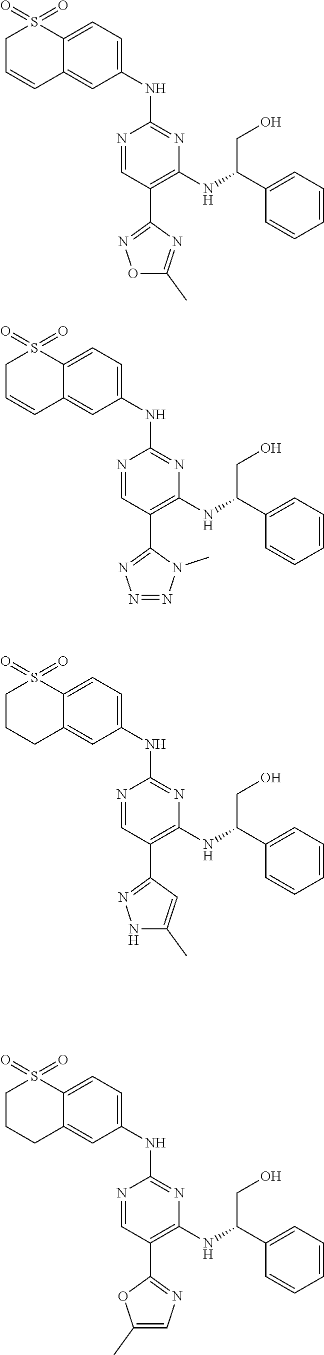 [SMILES: Cc1nc(-c2cnc(Nc3ccc4c(c3)C=CCS4(=O)=O)nc2N[C@H](CO)c2ccccc2)no1 ; Cn1nnnc1-c1cnc(Nc2ccc3c(c2)C=CCS3(=O)=O)nc1N[C@H](CO)c1ccccc1 ; Cc1cc(-c2cnc(Nc3ccc4c(c3)CCCS4(=O)=O)nc2N[C@H](CO)c2ccccc2)n[nH]1 ; Cc1cnc(-c2cnc(Nc3ccc4c(c3)CCCS4(=O)=O)nc2N[C@H](CO)c2ccccc2)o1] 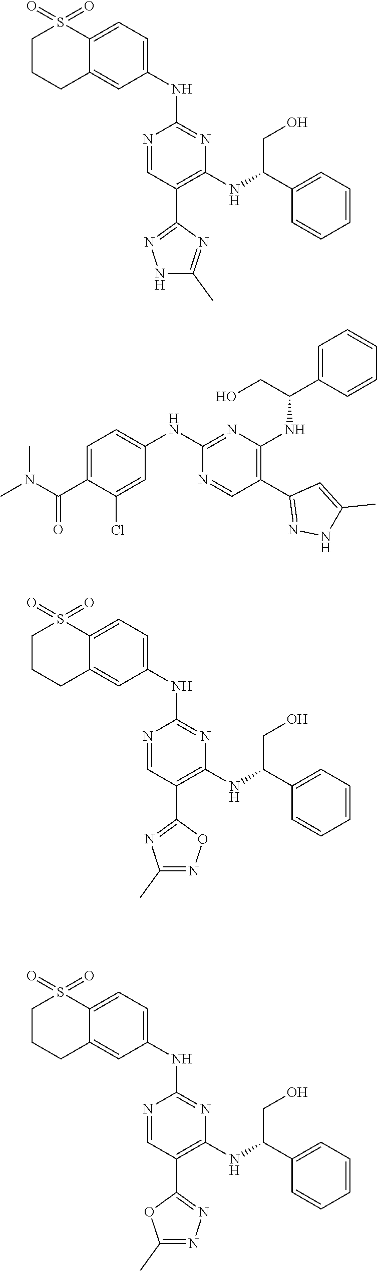 [SMILES: Cc1nc(-c2cnc(Nc3ccc4c(c3)CCCS4(=O)=O)nc2N[C@H](CO)c2ccccc2)n[nH]1 ; Cc1cc(-c2cnc(Nc3ccc(C(=O)N(C)C)c(Cl)c3)nc2N[C@H](CO)c2ccccc2)n[nH]1 ; Cc1noc(-c2cnc(Nc3ccc4c(c3)CCCS4(=O)=O)nc2N[C@H](CO)c2ccccc2)n1 ; Cc1nnc(-c2cnc(Nc3ccc4c(c3)CCCS4(=O)=O)nc2N[C@H](CO)c2ccccc2)o1] 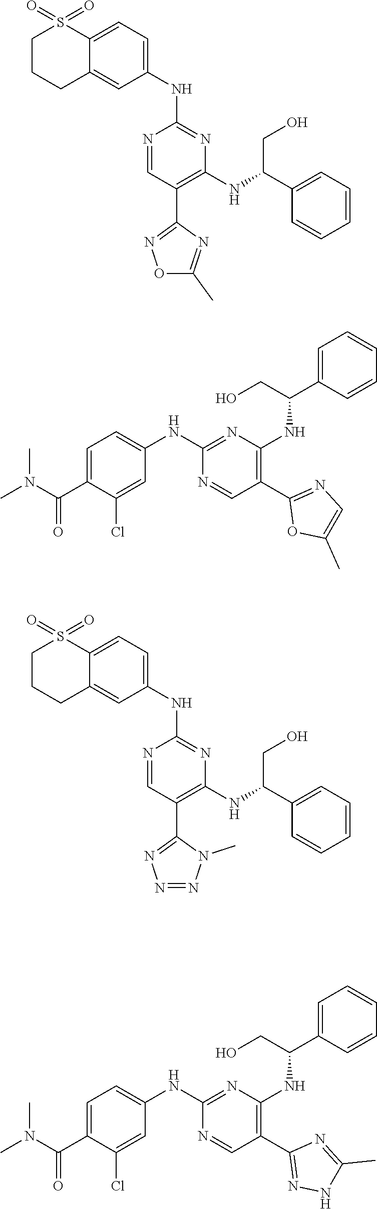 [SMILES: Cc1nc(-c2cnc(Nc3ccc4c(c3)CCCS4(=O)=O)nc2N[C@H](CO)c2ccccc2)no1 ; Cc1cnc(-c2cnc(Nc3ccc(C(=O)N(C)C)c(Cl)c3)nc2N[C@H](CO)c2ccccc2)o1 ; Cn1nnnc1-c1cnc(Nc2ccc3c(c2)CCCS3(=O)=O)nc1N[C@H](CO)c1ccccc1 ; Cc1nc(-c2cnc(Nc3ccc(C(=O)N(C)C)c(Cl)c3)nc2N[C@H](CO)c2ccccc2)n[nH]1] 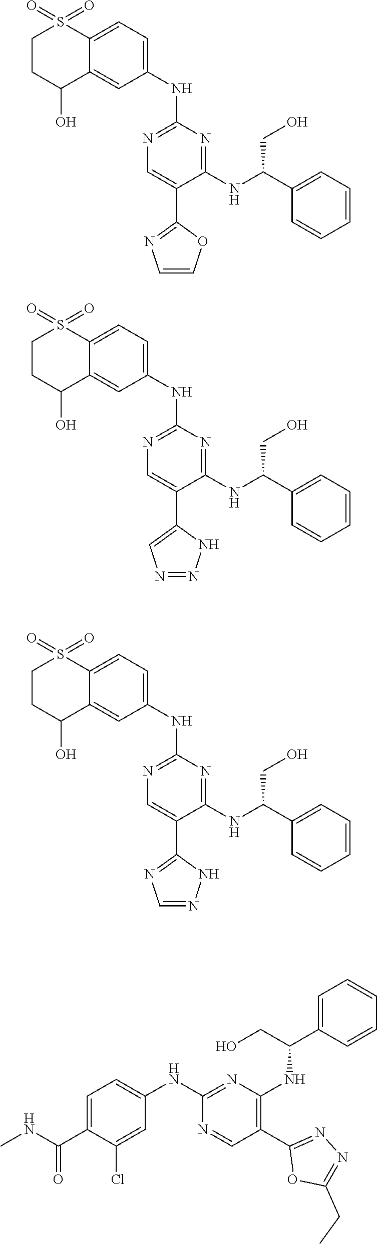 [SMILES: O=S1(=O)CCC(O)c2cc(Nc3ncc(-c4ncco4)c(N[C@H](CO)c4ccccc4)n3)ccc21 ; O=S1(=O)CCC(O)c2cc(Nc3ncc(-c4cnn[nH]4)c(N[C@H](CO)c4ccccc4)n3)ccc21 ; O=S1(=O)CCC(O)c2cc(Nc3ncc(-c4ncn[nH]4)c(N[C@H](CO)c4ccccc4)n3)ccc21 ; CCc1nnc(-c2cnc(Nc3ccc(C(=O)NC)c(Cl)c3)nc2N[C@H](CO)c2ccccc2)o1] 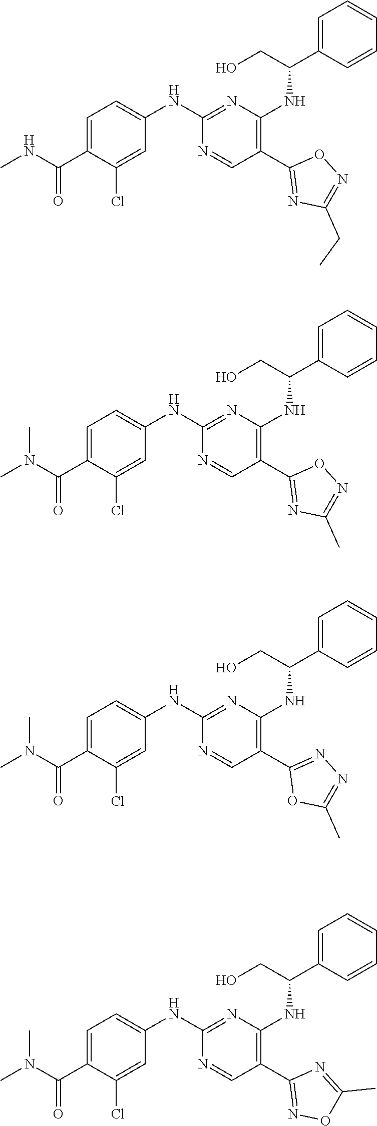 [SMILES: CCc1noc(-c2cnc(Nc3ccc(C(=O)NC)c(Cl)c3)nc2N[C@H](CO)c2ccccc2)n1 ; Cc1noc(-c2cnc(Nc3ccc(C(=O)N(C)C)c(Cl)c3)nc2N[C@H](CO)c2ccccc2)n1 ; Cc1nnc(-c2cnc(Nc3ccc(C(=O)N(C)C)c(Cl)c3)nc2N[C@H](CO)c2ccccc2)o1 ; Cc1nc(-c2cnc(Nc3ccc(C(=O)N(C)C)c(Cl)c3)nc2N[C@H](CO)c2ccccc2)no1] 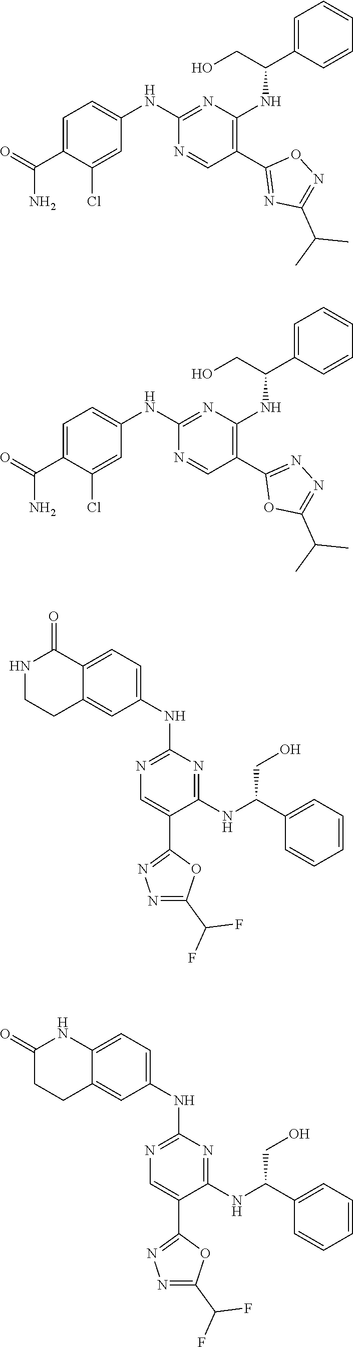 [SMILES: CC(C)c1noc(-c2cnc(Nc3ccc(C(N)=O)c(Cl)c3)nc2N[C@H](CO)c2ccccc2)n1 ; CC(C)c1nnc(-c2cnc(Nc3ccc(C(N)=O)c(Cl)c3)nc2N[C@H](CO)c2ccccc2)o1 ; O=C1NCCc2cc(Nc3ncc(-c4nnc(C(F)F)o4)c(N[C@H](CO)c4ccccc4)n3)ccc21 ; O=C1CCc2cc(Nc3ncc(-c4nnc(C(F)F)o4)c(N[C@H](CO)c4ccccc4)n3)ccc2N1] 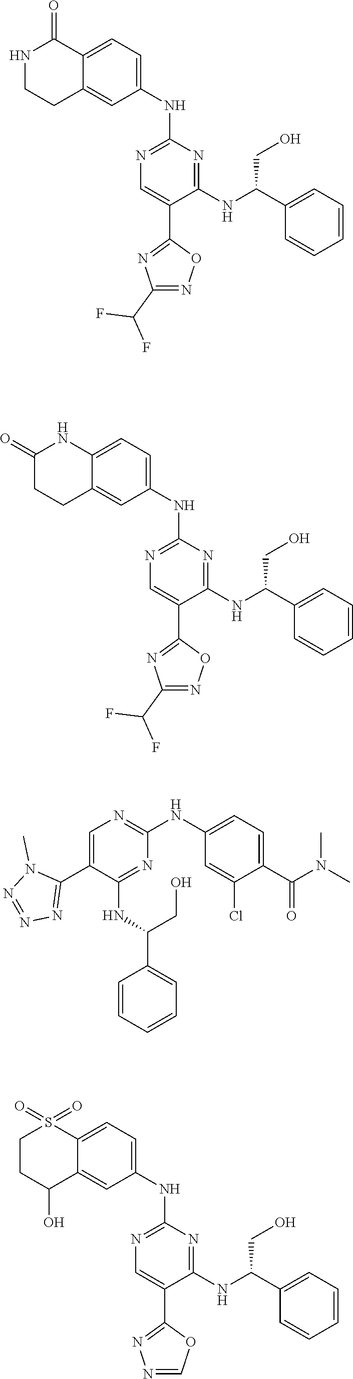 [SMILES: O=C1NCCc2cc(Nc3ncc(-c4nc(C(F)F)no4)c(N[C@H](CO)c4ccccc4)n3)ccc21 ; O=C1CCc2cc(Nc3ncc(-c4nc(C(F)F)no4)c(N[C@H](CO)c4ccccc4)n3)ccc2N1 ; CN(C)C(=O)c1ccc(Nc2ncc(-c3nnnn3C)c(N[C@H](CO)c3ccccc3)n2)cc1Cl ; O=S1(=O)CCC(O)c2cc(Nc3ncc(-c4nnco4)c(N[C@H](CO)c4ccccc4)n3)ccc21] 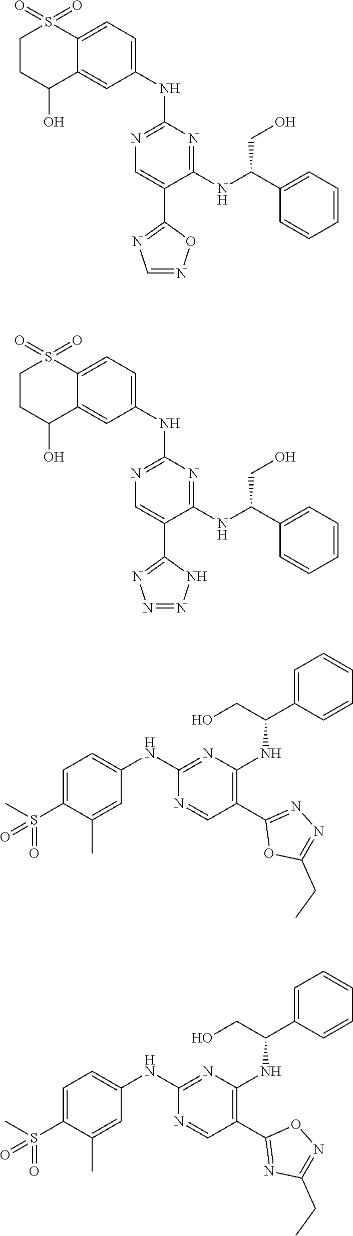 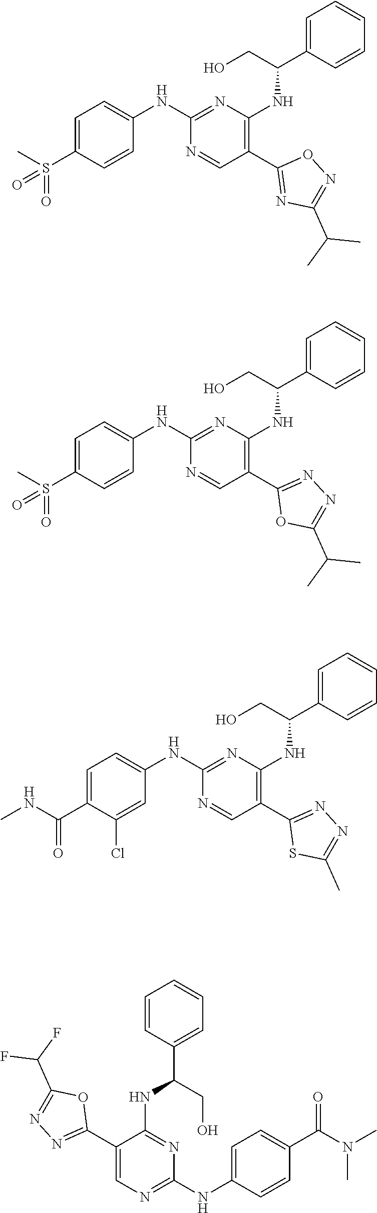 [SMILES: CC(C)c1noc(-c2cnc(Nc3ccc(S(C)(=O)=O)cc3)nc2N[C@H](CO)c2ccccc2)n1 ; CC(C)c1nnc(-c2cnc(Nc3ccc(S(C)(=O)=O)cc3)nc2N[C@H](CO)c2ccccc2)o1 ; CNC(=O)c1ccc(Nc2ncc(-c3nnc(C)s3)c(N[C@H](CO)c3ccccc3)n2)cc1Cl ; CN(C)C(=O)c1ccc(Nc2ncc(-c3nnc(C(F)F)o3)c(N[C@H](CO)c3ccccc3)n2)cc1] 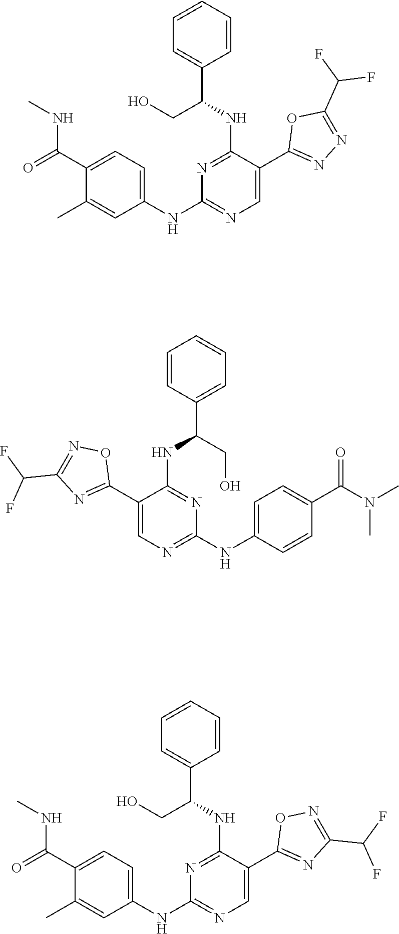 [SMILES: CNC(=O)c1ccc(Nc2ncc(-c3nnc(C(F)F)o3)c(N[C@H](CO)c3ccccc3)n2)cc1C ; CN(C)C(=O)c1ccc(Nc2ncc(-c3nc(C(F)F)no3)c(N[C@H](CO)c3ccccc3)n2)cc1 ; CNC(=O)c1ccc(Nc2ncc(-c3nc(C(F)F)no3)c(N[C@H](CO)c3ccccc3)n2)cc1C] 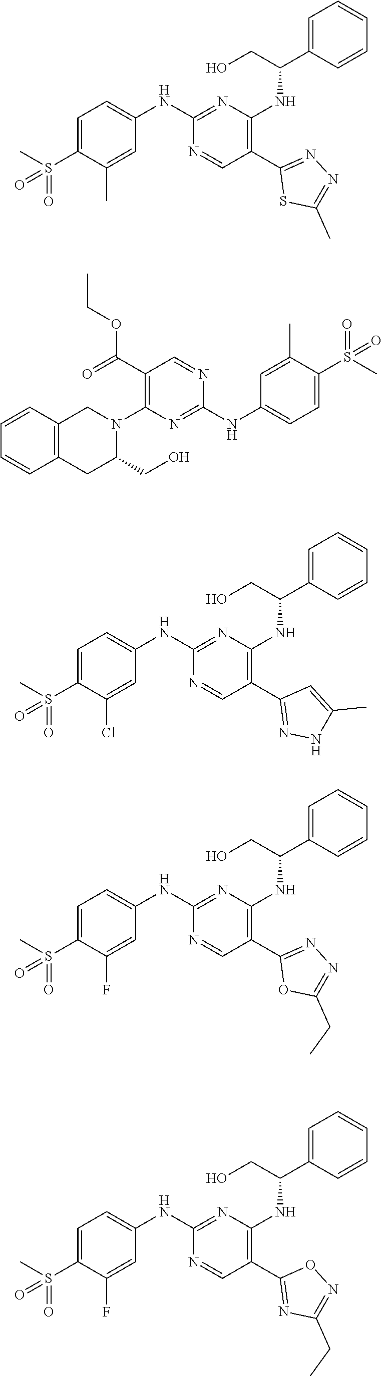 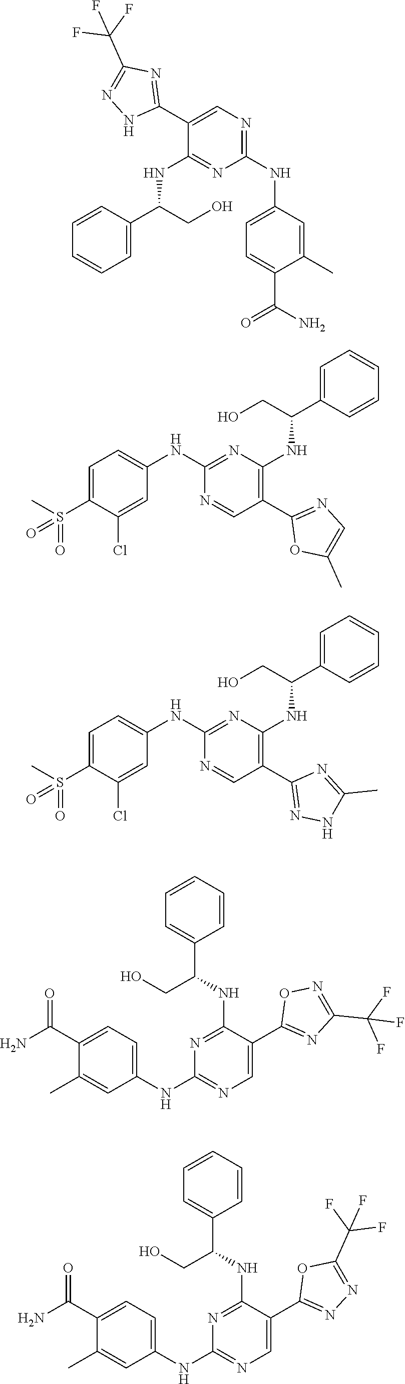 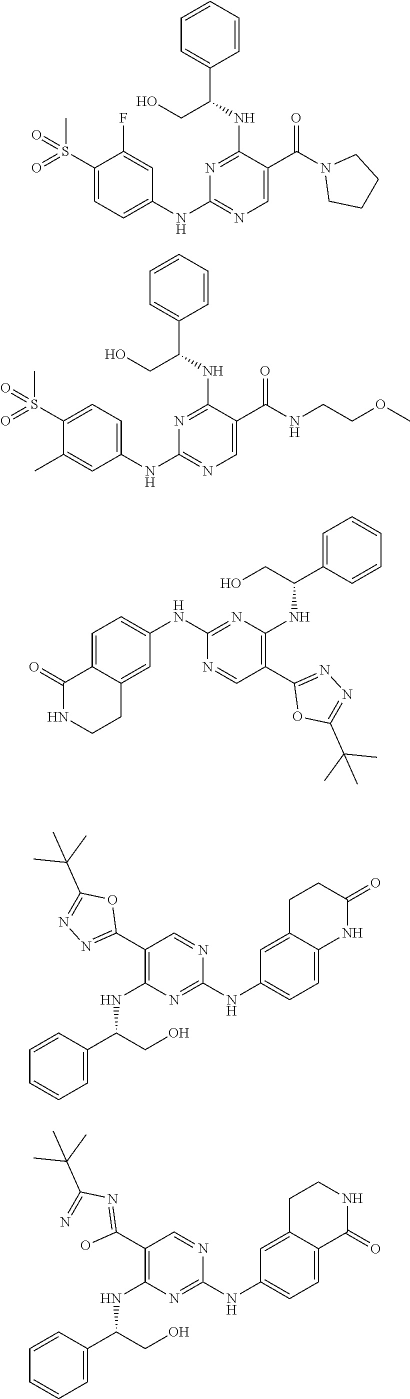 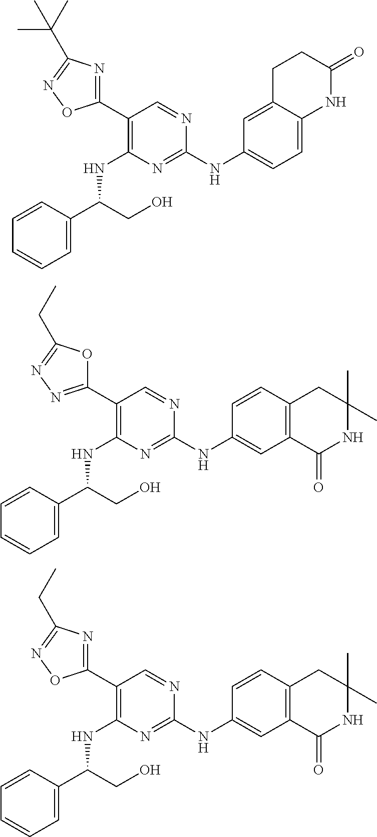 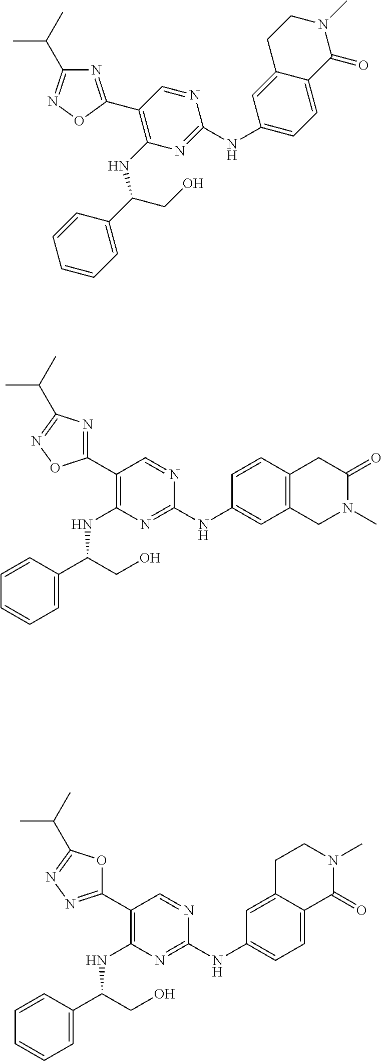 [SMILES: CC(C)c1noc(-c2cnc(Nc3ccc4c(c3)CCN(C)C4=O)nc2N[C@H](CO)c2ccccc2)n1 ; CC(C)c1noc(-c2cnc(Nc3ccc4c(c3)CN(C)C(=O)C4)nc2N[C@H](CO)c2ccccc2)n1 ; CC(C)c1nnc(-c2cnc(Nc3ccc4c(c3)CCN(C)C4=O)nc2N[C@H](CO)c2ccccc2)o1] 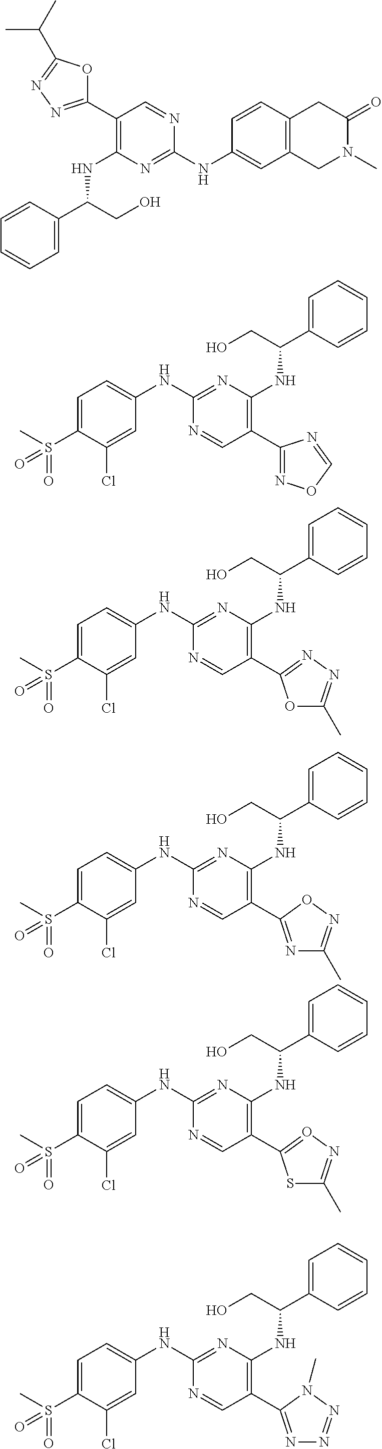 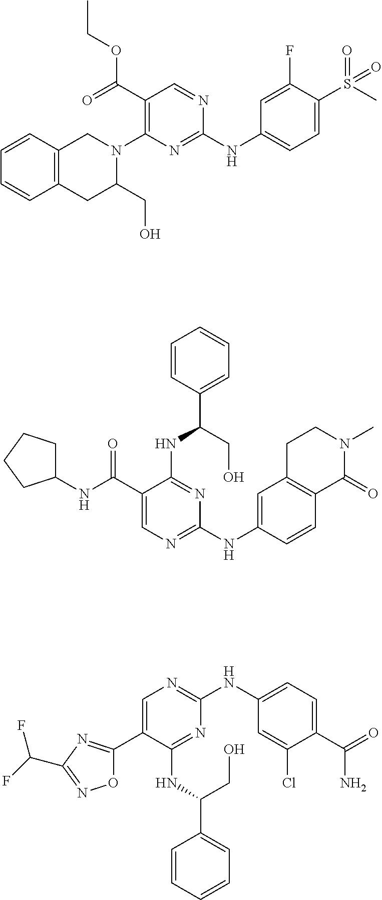 [SMILES: CCOC(=O)c1cnc(Nc2ccc(S(C)(=O)=O)c(F)c2)nc1N1Cc2ccccc2CC1CO ; CN1CCc2cc(Nc3ncc(C(=O)NC4CCCC4)c(N[C@H](CO)c4ccccc4)n3)ccc2C1=O ; NC(=O)c1ccc(Nc2ncc(-c3nc(C(F)F)no3)c(N[C@H](CO)c3ccccc3)n2)cc1Cl] 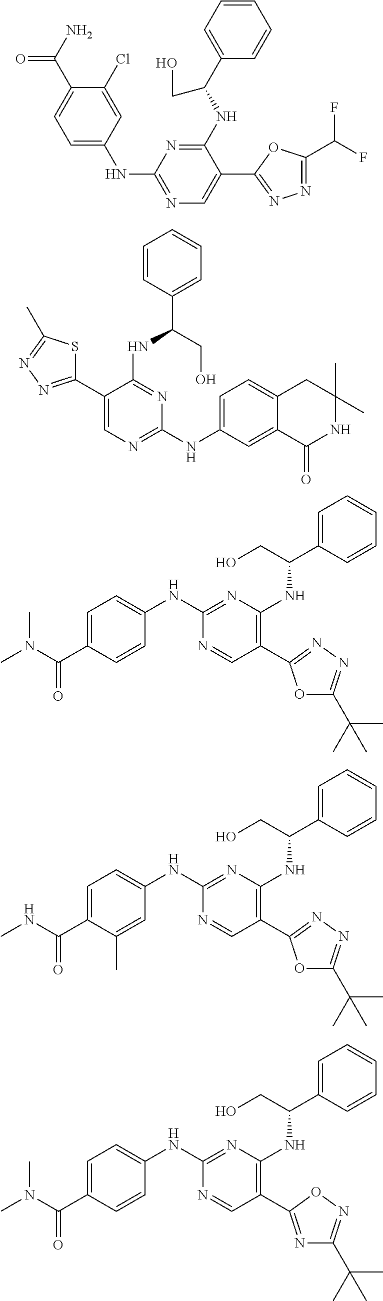 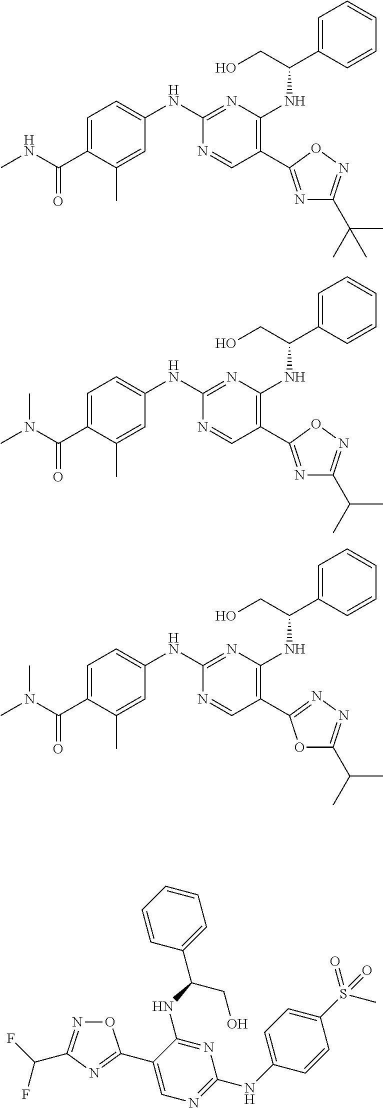 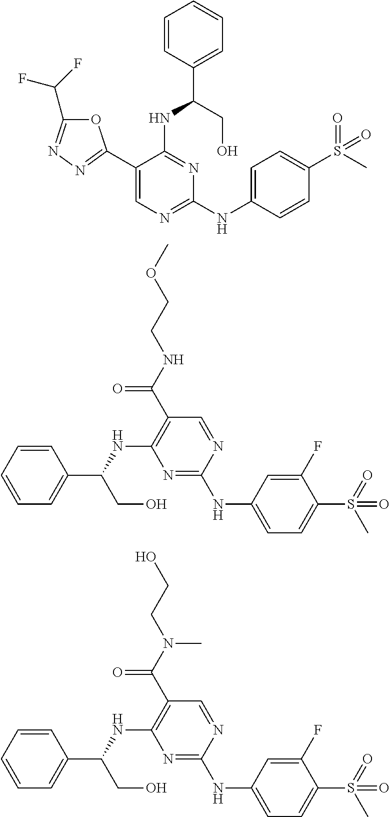 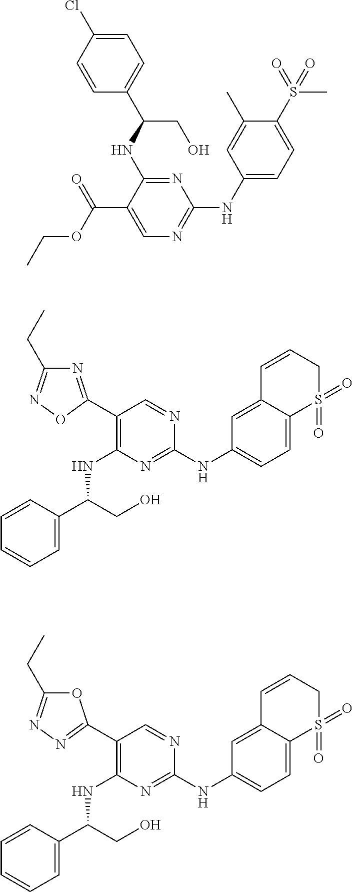 [SMILES: CCOC(=O)c1cnc(Nc2ccc(S(C)(=O)=O)c(C)c2)nc1N[C@H](CO)c1ccc(Cl)cc1 ; CCc1noc(-c2cnc(Nc3ccc4c(c3)C=CCS4(=O)=O)nc2N[C@H](CO)c2ccccc2)n1 ; CCc1nnc(-c2cnc(Nc3ccc4c(c3)C=CCS4(=O)=O)nc2N[C@H](CO)c2ccccc2)o1] 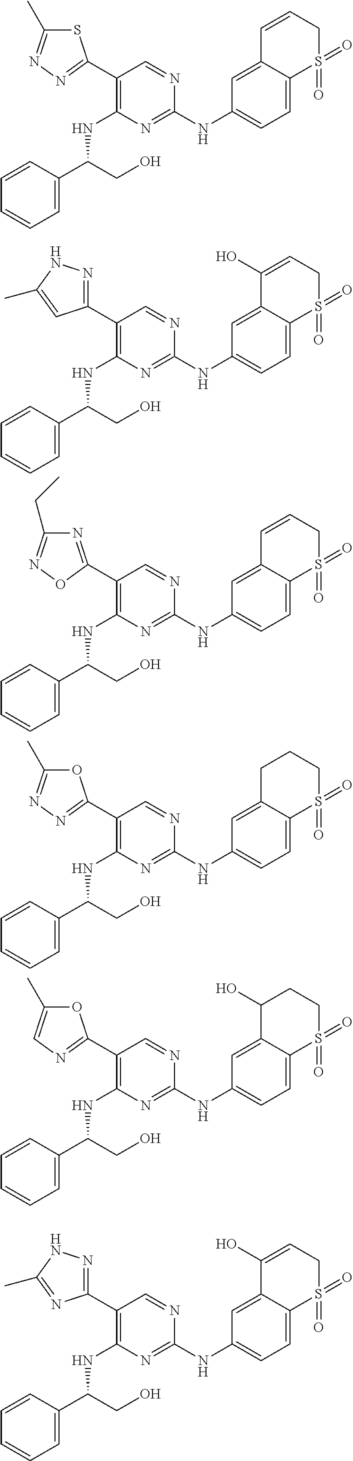 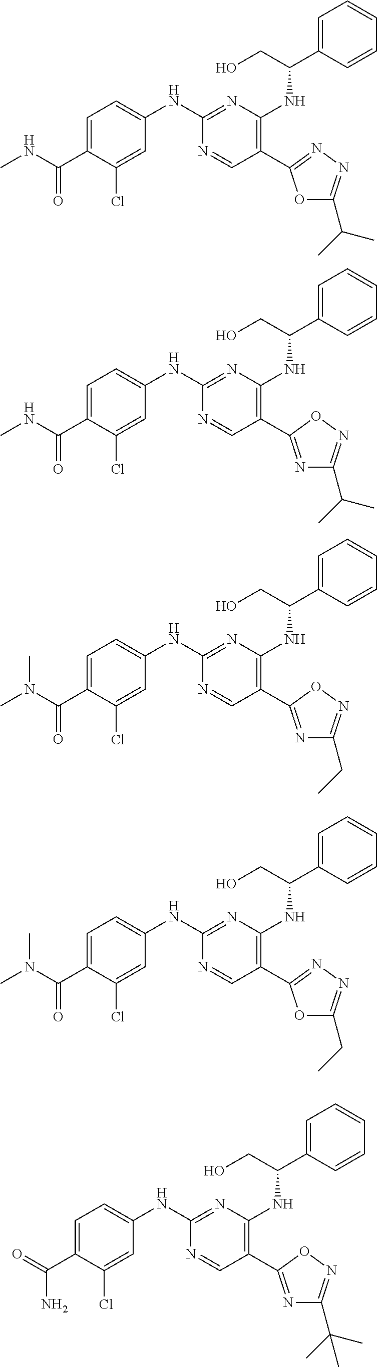 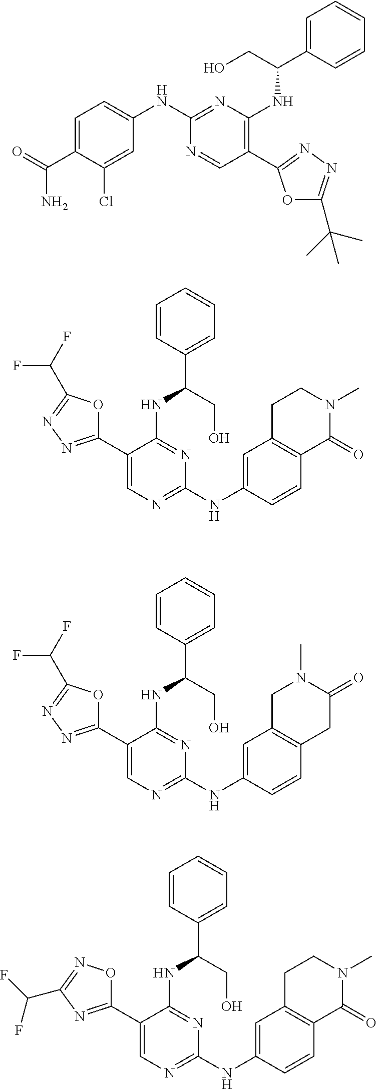 [SMILES: CC(C)(C)c1nnc(-c2cnc(Nc3ccc(C(N)=O)c(Cl)c3)nc2N[C@H](CO)c2ccccc2)o1 ; CN1CCc2cc(Nc3ncc(-c4nnc(C(F)F)o4)c(N[C@H](CO)c4ccccc4)n3)ccc2C1=O ; CN1Cc2cc(Nc3ncc(-c4nnc(C(F)F)o4)c(N[C@H](CO)c4ccccc4)n3)ccc2CC1=O ; CN1CCc2cc(Nc3ncc(-c4nc(C(F)F)no4)c(N[C@H](CO)c4ccccc4)n3)ccc2C1=O] 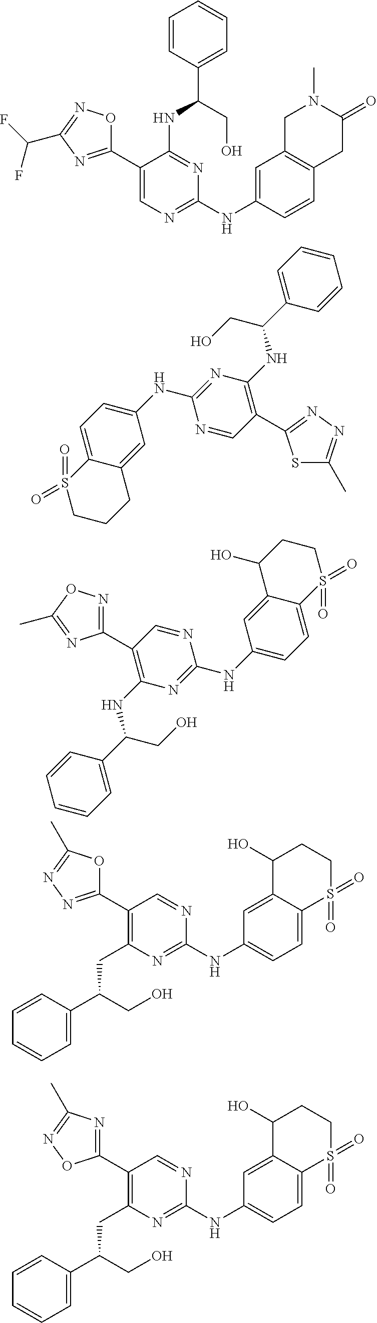 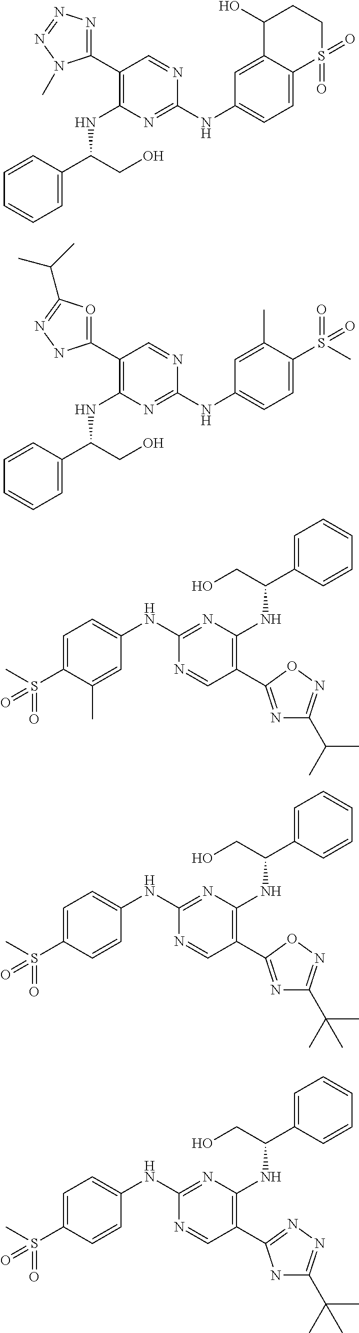 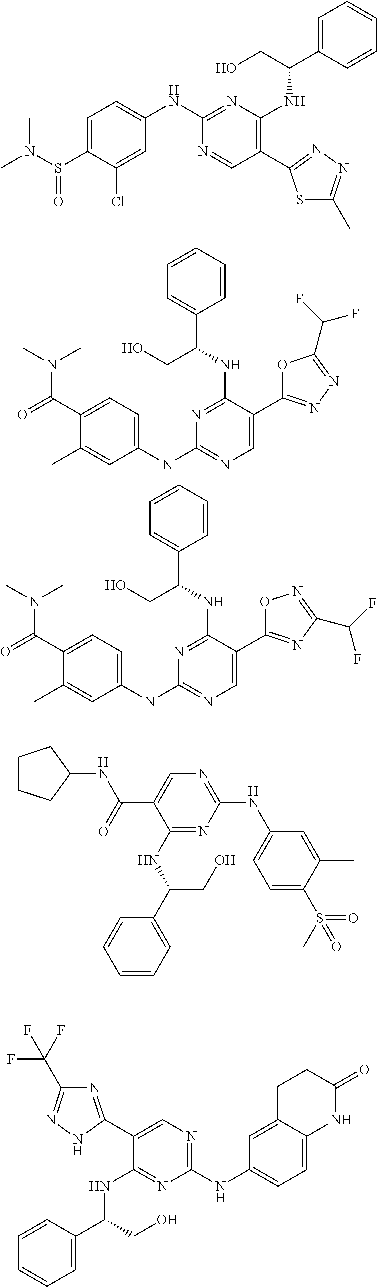 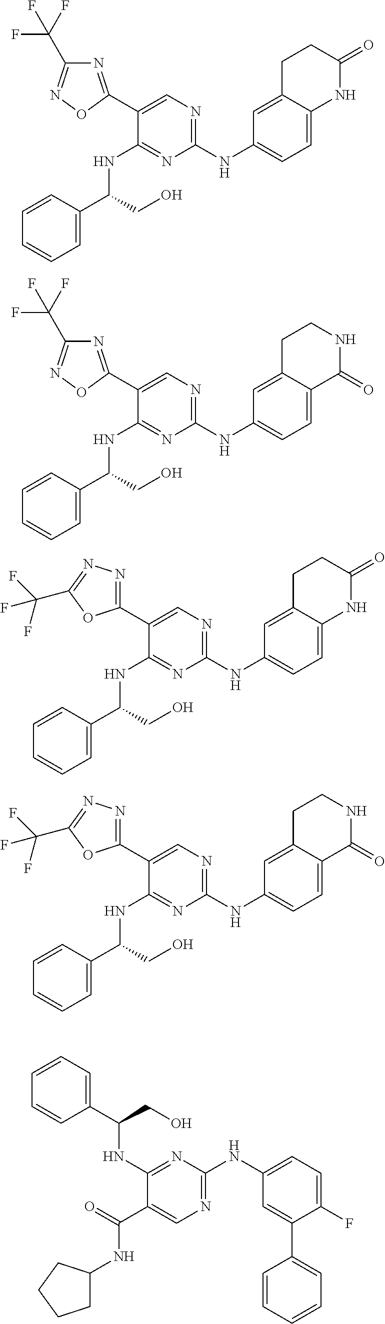 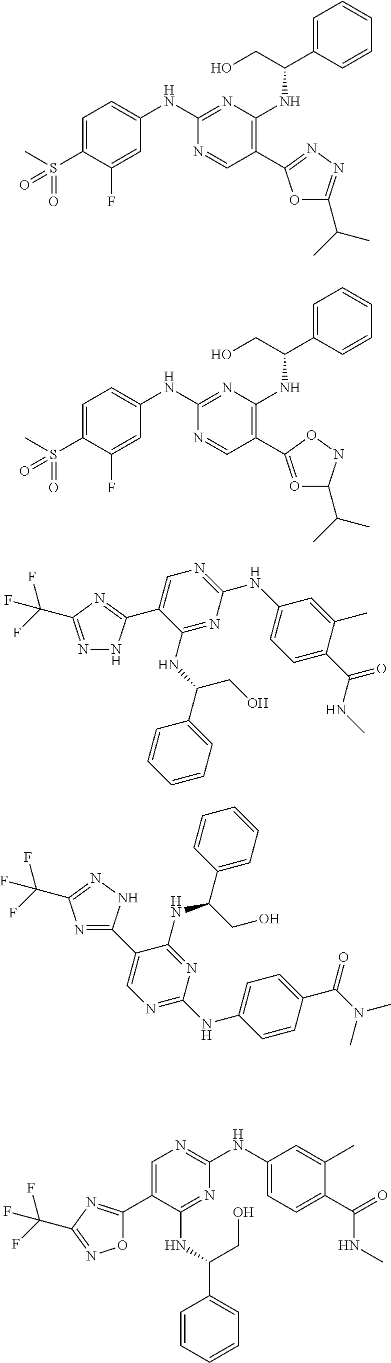 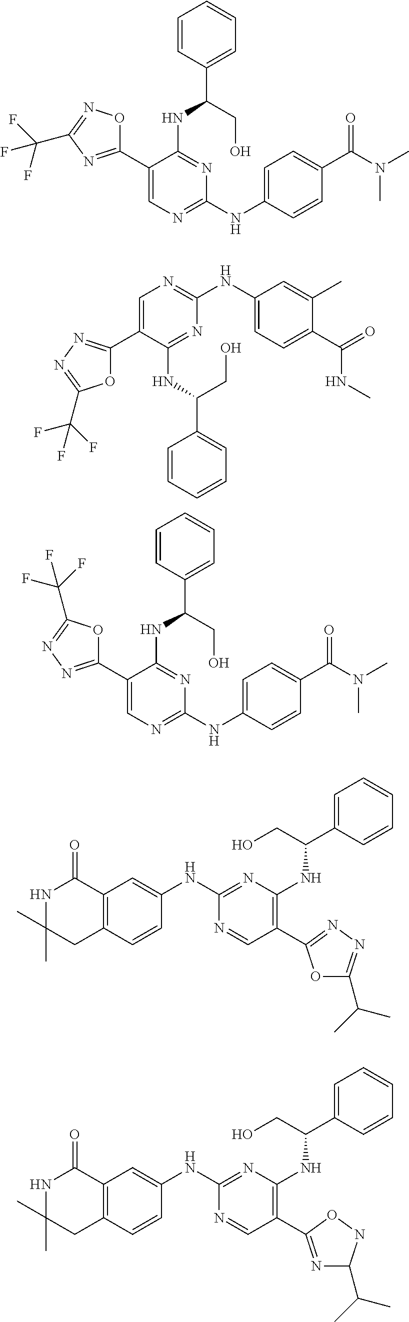 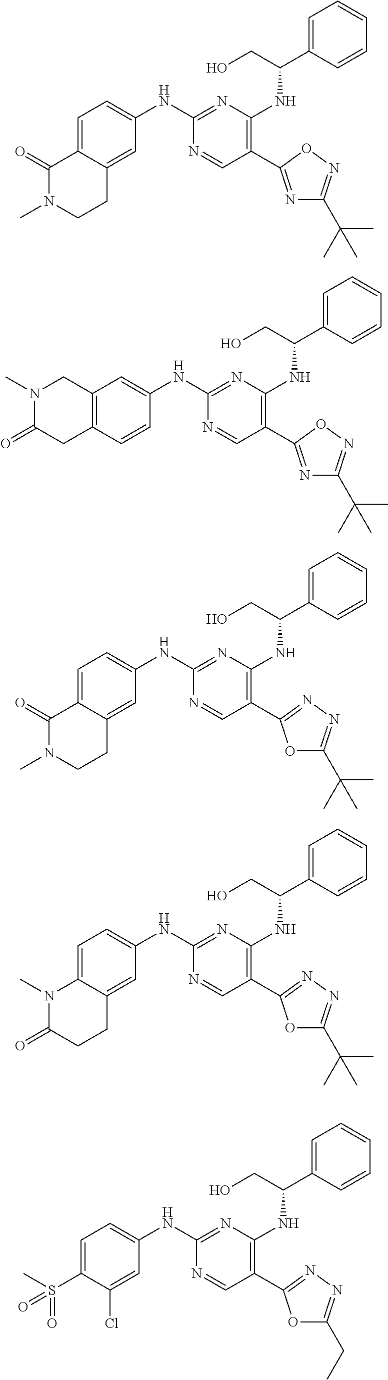 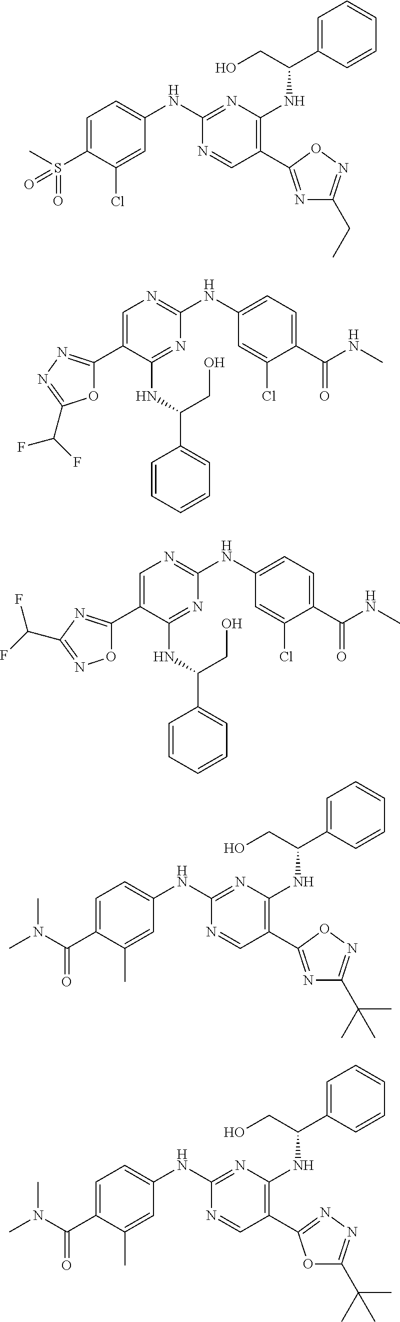 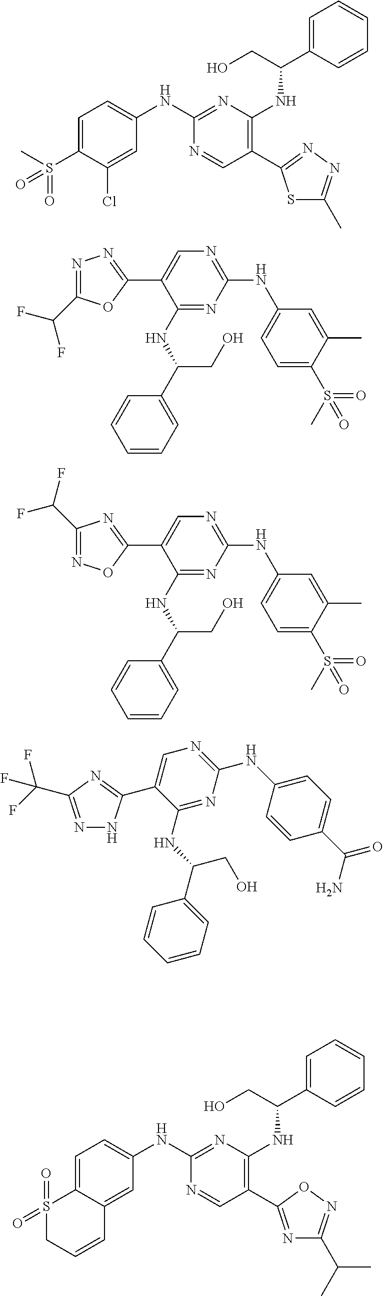 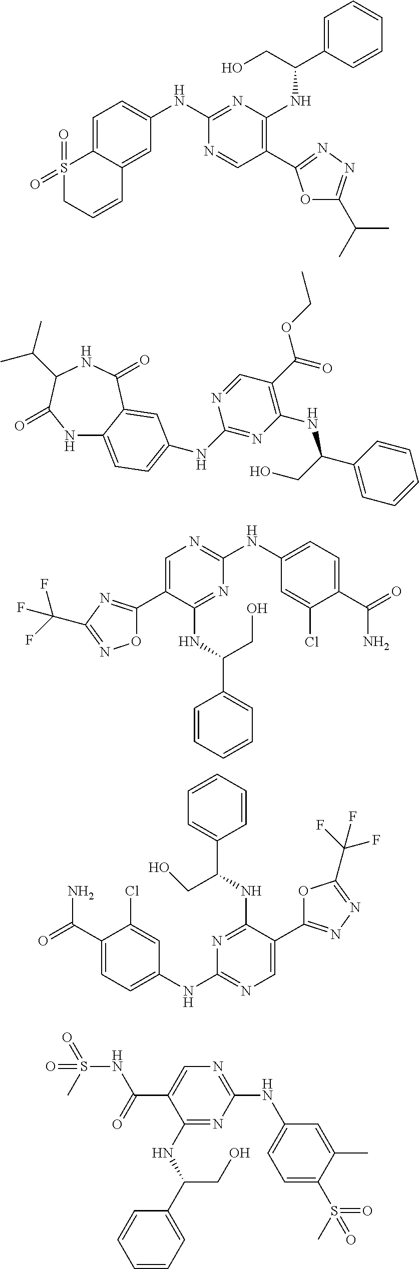 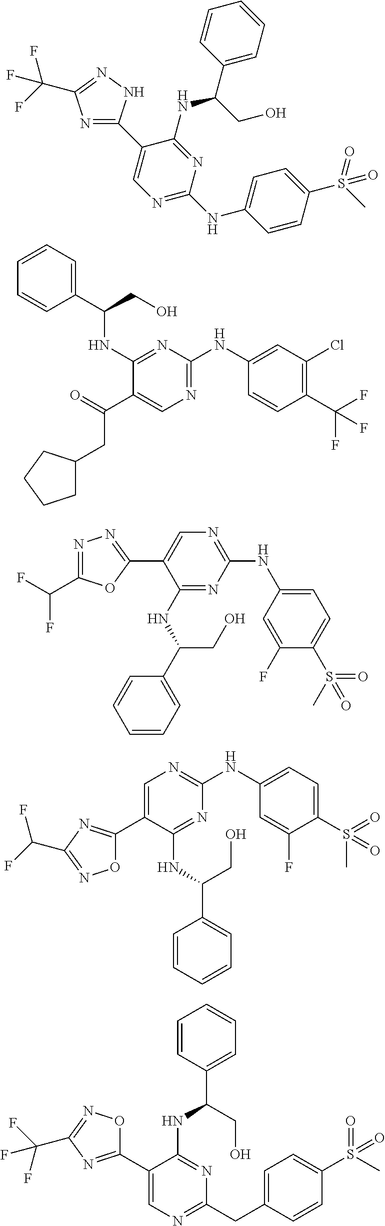 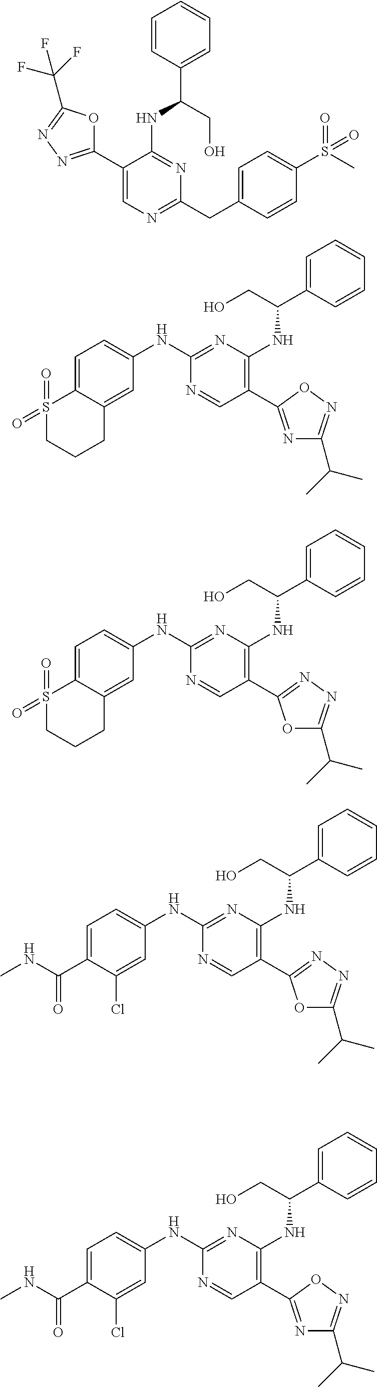 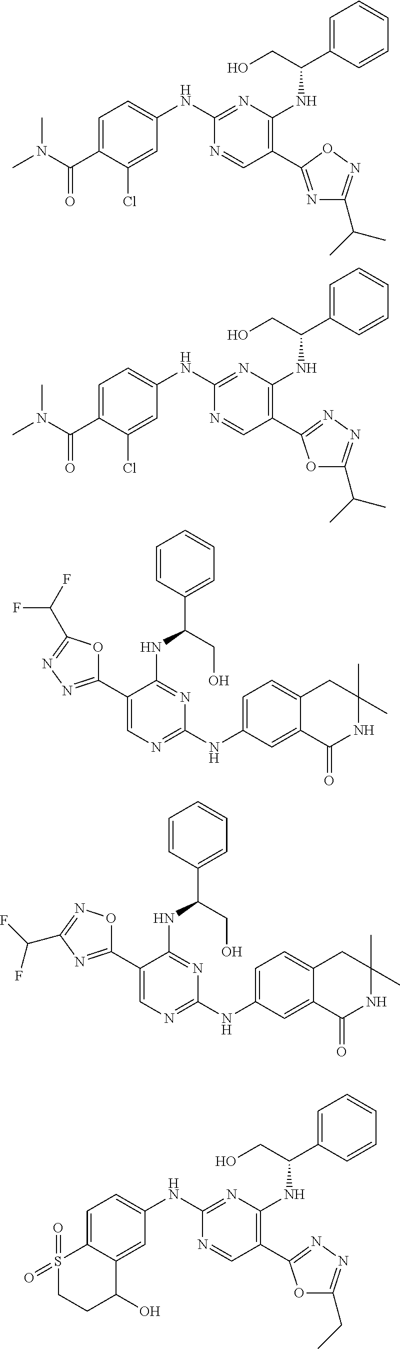 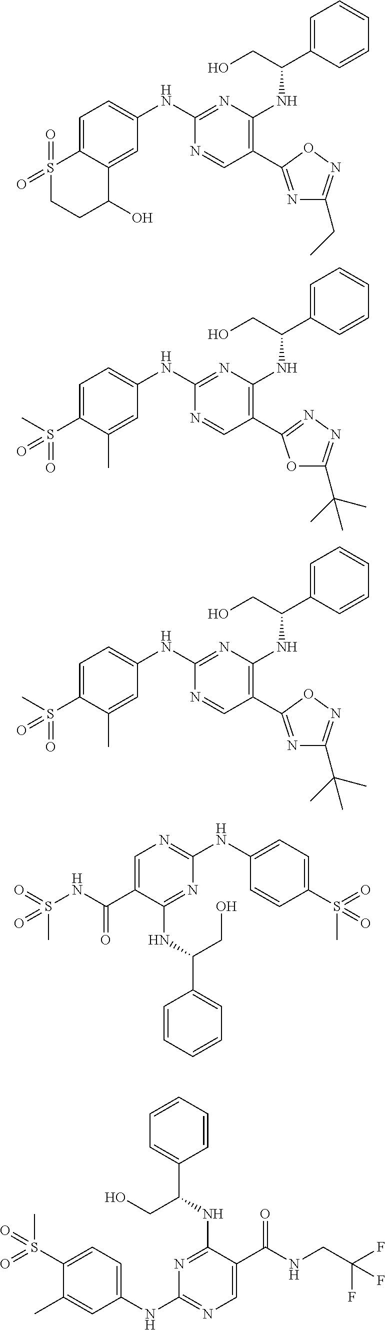 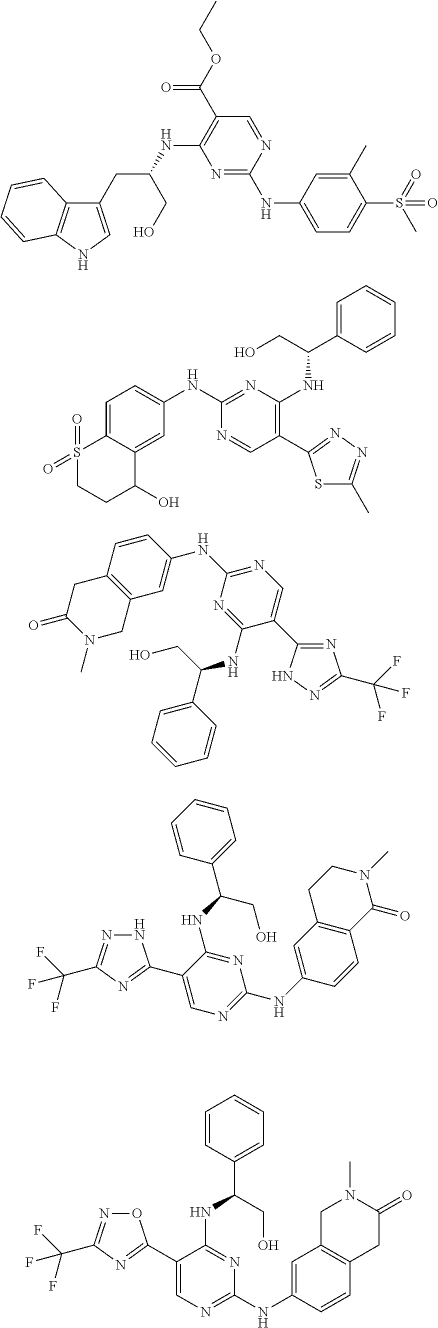 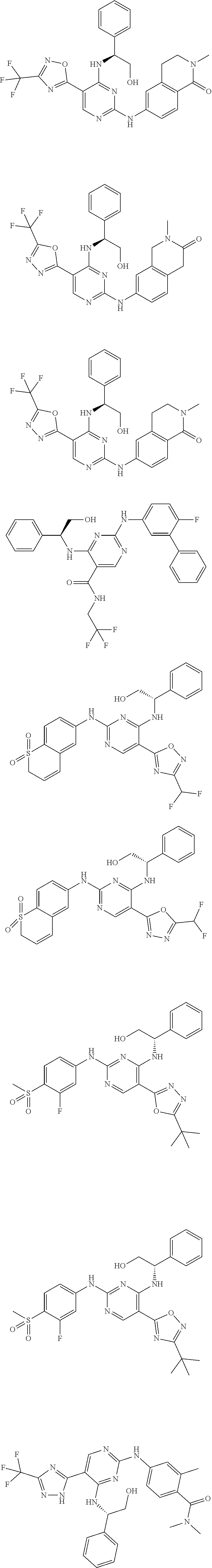 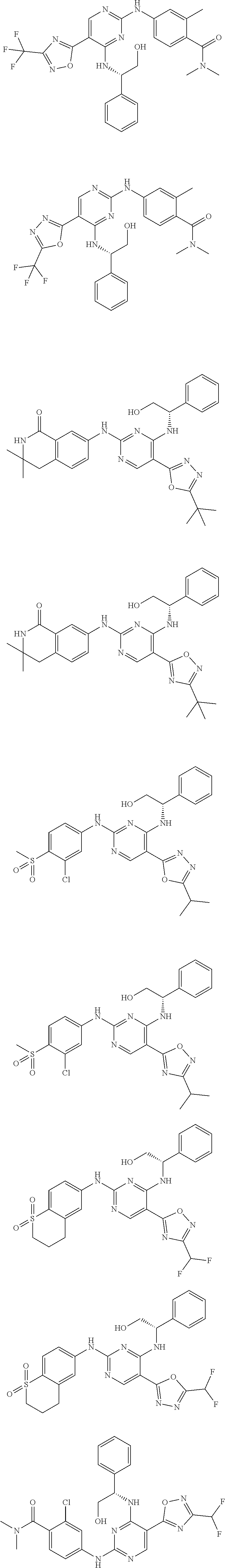 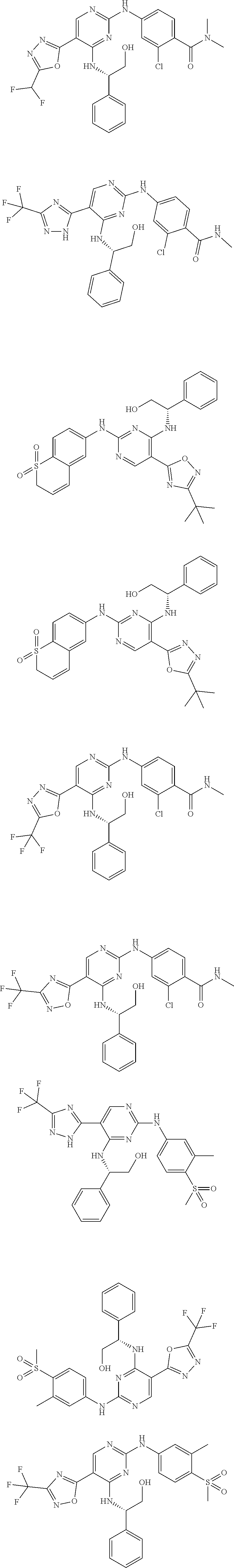 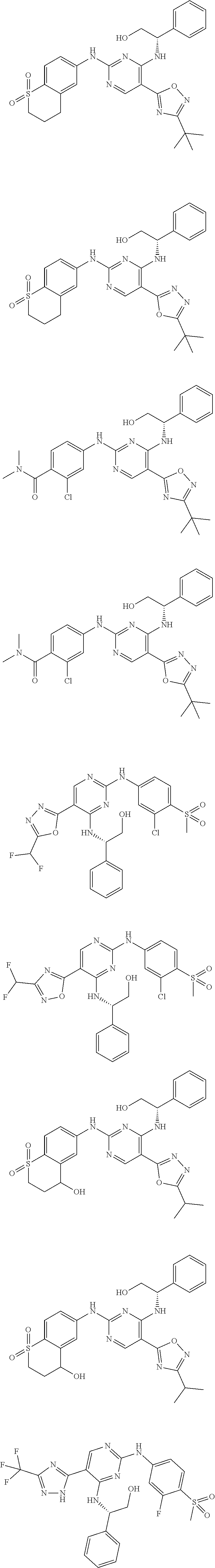 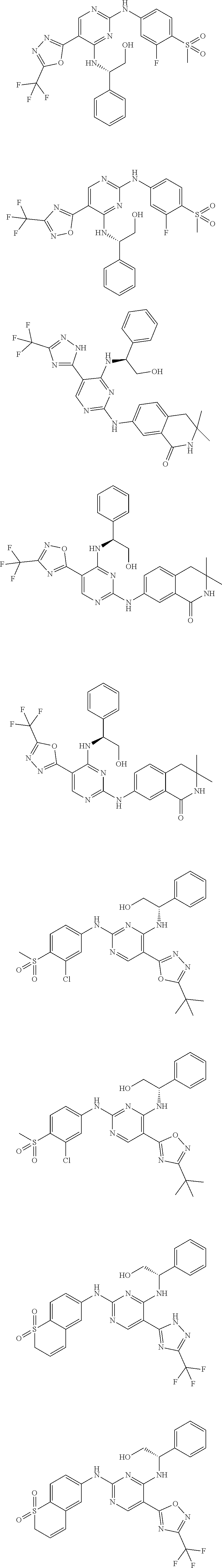 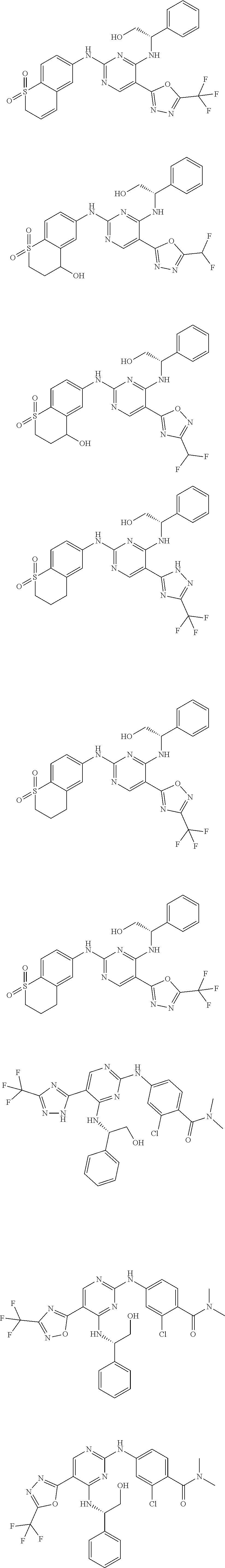 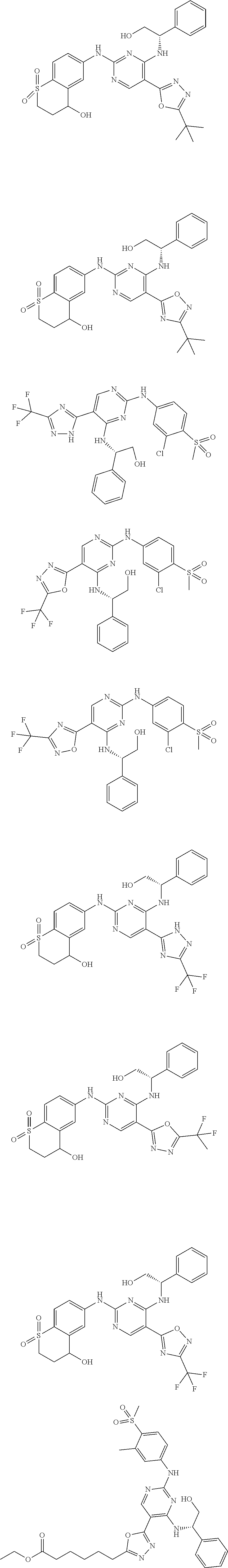 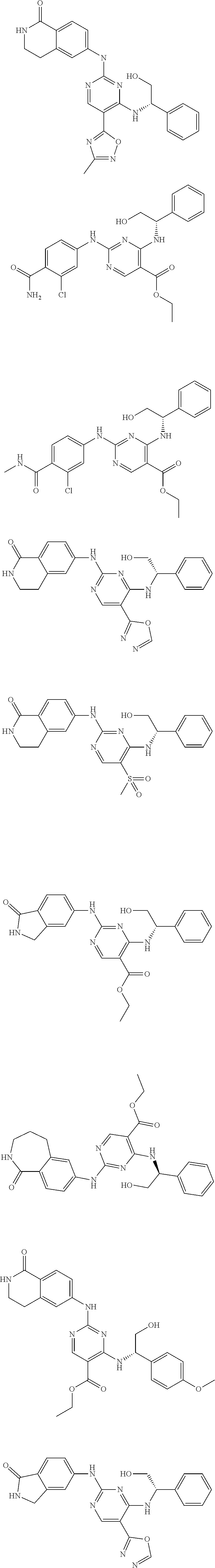 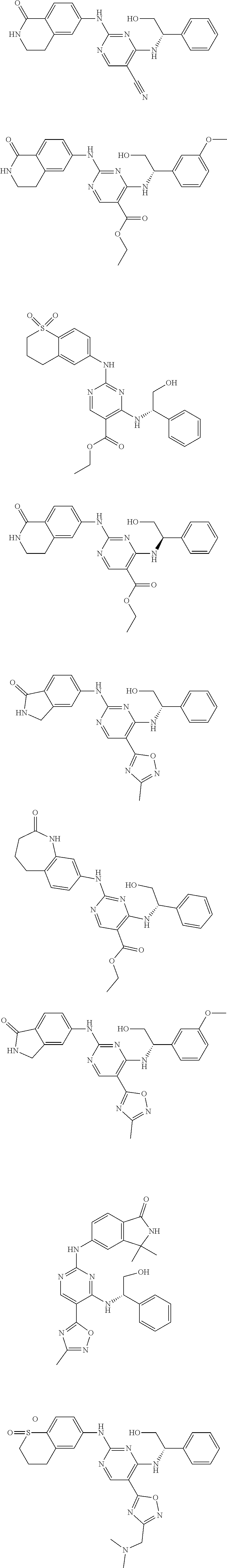 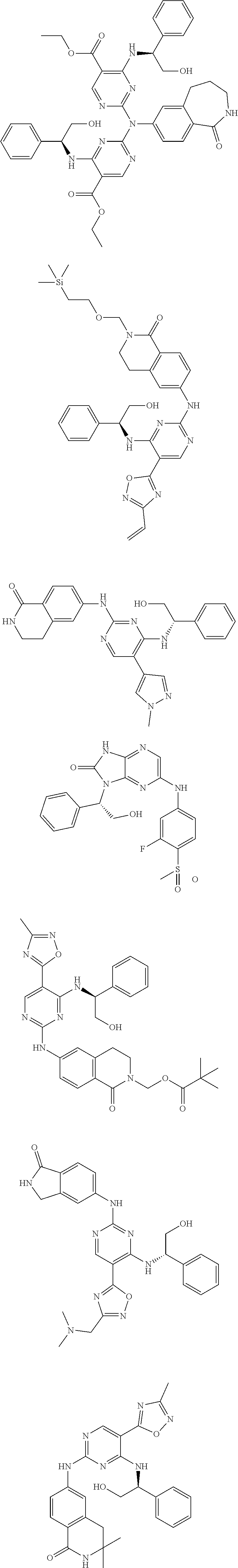 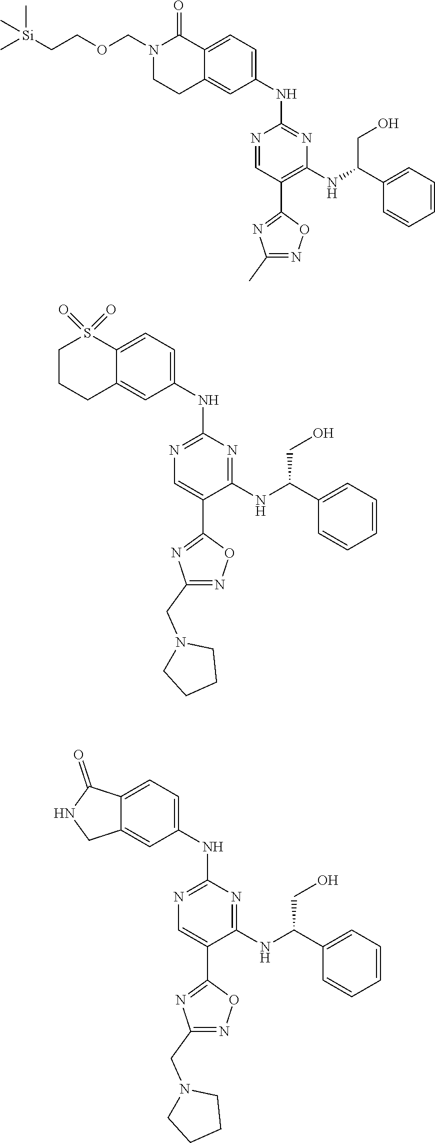 [SMILES: Cc1noc(-c2cnc(Nc3ccc4c(c3)CCN(COCC[Si](C)(C)C)C4=O)nc2N[C@H](CO)c2ccccc2)n1 ; O=S1(=O)CCCc2cc(Nc3ncc(-c4nc(CN5CCCC5)no4)c(N[C@H](CO)c4ccccc4)n3)ccc21 ; O=C1NCc2cc(Nc3ncc(-c4nc(CN5CCCC5)no4)c(N[C@H](CO)c4ccccc4)n3)ccc21]
or a pharmaceutically acceptable salt, solvate, stereoisomer, or tautomer thereof.

14. The compound of claim 1 selected from the group consisting of:
4-[[(1S)-2-hydroxy-1-phenyl-ethyl]amino]-N-methyl-2-[(2-methyl-1-oxo-3,4-dihydroisoquinolin-6-yl)amino]pyrimidine-5-carboxamide;
ethyl 4-[[(1S)-2-hydroxy-1-phenyl-ethyl]amino]-2-[(1-oxo-3,4-dihydro-2H-isoquinolin-6-yl)amino]pyrimidine-5-carboxylate;
(S)-4-((2-hydroxy-1-phenylethyl)amino)-N-isopropyl-2-((1-isopropyl-1H-pyrazolo[4,3-c]pyridin-6-yl)amino)pyrimidine-5-carboxamide;
ethyl 2-(3-fluoro-4-methylsulfonyl-anilino)-4-[[(1S)-2-hydroxy-1-phenyl-ethyl]amino]-pyrimidine-5-carboxylate;
2-(3-fluoro-4-methylsulfonyl-anilino)-4-[[(1S)-2-hydroxy-1-phenyl-ethyl]amino]-N-methyl-pyrimidine-5-carboxamide;
4-[[(1S)-2-hydroxy-1-phenyl-ethyl]amino]-N-methyl-2-(3-methyl-4-methylsulfonyl-anilino)pyrimidine-5-carboxamide;
ethyl 4-[[(1S)-2-hydroxy-1-phenyl-ethyl]amino]-2-[(2-methyl-1-oxo-3,4-dihydroisoquinolin-6-yl)amino]pyrimidine-5-carboxylate;
ethyl 4-[[(1S)-2-hydroxy-1-phenyl-ethyl]amino]-2-(3-methyl-4-methylsulfonyl-anilino)pyrimidine-5-carboxylate;
N-ethyl-4-[[(1S)-2-hydroxy-1-phenyl-ethyl]amino]-2-(3-methyl-4-methylsulfonyl-anilino)pyrimidine-5-carboxamide;
4-[[(1S)-2-hydroxy-1-phenyl-ethyl]amino]-2-(3-methyl-4-methylsulfonyl-anilino)-N-(2,2,2-trifluoroethyl)-pyrimidine-5-carboxamide;
N-ethyl-4-[[(1S)-2-hydroxy-1-phenyl-ethyl]amino]-2-[(2-methyl-1-oxo-3,4-dihydroisoquinolin-6-yl)amino]pyrimidine-5-carboxamide;
ethyl 4-[[(1S)-2-hydroxy-1-phenyl-ethyl]amino]-2-(3-methoxy-4-methylsulfonyl-anilino)pyrimidine-5-carboxylate;
ethyl 2-(4-ethylsulfonylanilino)-4-[[(1S)-2-hydroxy-1-phenyl-ethyl]amino]pyrimidine-5-carboxylate;
ethyl 4-[[(1S)-2-hydroxy-1-phenyl-ethyl]amino]-2-(4-methylsulfonylanilino)pyrimidine-5-carboxylate;
(2S)-2-[[2-(3-methyl-4-methylsulfonyl-anilino)-5-(3-methyl-1,2,4-oxadiazol-5-yl)pyrimidin-4-yl]amino]-2-phenyl-ethanol;
2-(3-fluoro-4-methylsulfonyl-anilino)-4-[[(1S)-2-hydroxy-1-phenyl-ethyl]amino]pyrimidine-5-carboxamide;
(2S)-2-[[2-(3-fluoro-4-methylsulfonyl-anilino)-5-(1,3,4-oxadizol-2-yl)pyrimidin-4-yl]amino]-2-phenyl-ethanol;
(2S)-2-[[2-(3-fluoro-4-methylsulfonyl-anilino)-5-(3-methyl-1,2,4-oxadiazol-5-yl)pyrimidin-4-yl]amino]-2-phenyl-ethanol;
2-hydroxyethyl 2-(3-fluoro-4-methylsulfonyl-anilino)-4-[[(1S)-2-hydroxy-1-phenyl-ethyl]amino]pyrimidine-5-carboxylate;
ethyl 2-[(2-ethyl-1-oxo-3,4-dihydroisoquinolin-6-yl)amino]-4-[[(1S)-2-hydroxy-1-phenyl-ethyl]amino]pyrimidine-5-carboxylate;
ethyl 2-[4-(dimethylcarbamoyl)anilino]-4-[[(1S)-2-hydroxy-1-phenyl-ethyl]amino]pyrimidine-5-carboxylate;
or a pharmaceutically acceptable salt, stereo isomer, solvate, or tautomer thereof.

15. A pharmaceutical composition comprising the compound or a pharmaceutically acceptable salt, solvate, stereoisomer, or tautomer thereof of any one of claims 1-14, and a pharmaceutically acceptable carrier.

16. The pharmaceutical composition of claim 15, further comprising an additional pharmaceutically active agent.

17. A method of inhibiting a hematopoietic progenitor kinase 1 (HPK1), comprising of administering to a subject a compound of any one of claims 1-14 or the pharmaceutical composition of any one of claim 15 or 16.

18. A method of treating a disease or disorder associated with the inhibition of hematopoietic progenitor kinase 1 (HPK1), comprising of administering to a subject a compound of any one of claims 1-14 or the pharmaceutical composition of any one of claim 15 or 16.

19. A method of treating a disease, disorder, or condition, comprising of administering to a subject in need of a treatment a compound of any one of claims 1-14 or the pharmaceutical composition of any one of claim 15 or 16.

20. The method of claim 19, wherein the disease, disorder, or condition is selected from cancer, an autoimmune disease, an inflammatory disease, a viral infection, male fertility control, a benign hyperplasia, sepsis, a vascular disorder, an atherosclerotic disease, and a neurodegenerative disorder.

21. The method of claim 20, wherein the disease, disorder, or condition is cancer selected from bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, marginal zone lymphoma (MZL), follicular lymphoma (FL), diffuse large B-cell lymphoma (DLBCL), and chronic lymphocytic leukemia/small lymphocytic lymphoma (CLL/SLL).

22. The method of any one of claims 17-21, wherein the subject is a mammal.

23. The method of claim 22, wherein the subject is a human.
